SEQUENCE LISTING (149 sequences)

SEQ ID NO: 1 - protein, 446 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 116 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 5 aa, synthetic construct
DYGMH

SEQ ID NO: 4 - protein, 17 aa, synthetic construct
YISSGSSTIYYADTVKG

SEQ ID NO: 5 - protein, 7 aa, synthetic construct
RGLLLDY

SEQ ID NO: 6 - protein, 19 aa, synthetic construct
MGWSCIILFLVATATGVHS

SEQ ID NO: 7 - protein, 218 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 111 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 9 - protein, 15 aa, synthetic construct
RASKSVSTSSYSYMH

SEQ ID NO: 10 - protein, 7 aa, synthetic construct
YASYLES

SEQ ID NO: 11 - protein, 9 aa, synthetic construct
QHSREFPWT

SEQ ID NO: 12 - protein, 19 aa, synthetic construct
MGWSCIILFLVATATGVHS

SEQ ID NO: 13 - protein, 446 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 116 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 15 - protein, 5 aa, synthetic construct
NYGMH

SEQ ID NO: 16 - protein, 5 aa, synthetic construct
  VARIANT 1: note = D can be replaced by N, R or K
DYGMH

SEQ ID NO: 17 - protein, 17 aa, synthetic construct
YISSGSSTIYYADTVKG

SEQ ID NO: 18 - protein, 7 aa, synthetic construct
RGLLLDY

SEQ ID NO: 19 - protein, 19 aa, synthetic construct
MGWSCIILFLVATATGVHS

SEQ ID NO: 20 - protein, 218 aa, synthetic construct; identical to SEQ ID NO: 7

SEQ ID NO: 21 - protein, 111 aa, synthetic construct; identical to SEQ ID NO: 8

SEQ ID NO: 22 - protein, 15 aa, synthetic construct
RASKSVSTSSYSYMH

SEQ ID NO: 23 - protein, 7 aa, synthetic construct
YASYLES

SEQ ID NO: 24 - protein, 9 aa, synthetic construct
QHSREFPWT

SEQ ID NO: 25 - protein, 19 aa, synthetic construct
MGWSCIILFLVATATGVHS

SEQ ID NO: 26 - protein, 17 aa, synthetic construct
YISSGSSTIYYADSVKG

SEQ ID NO: 27 - protein, 17 aa, synthetic construct
YISSSSSTIYYADSVKG

SEQ ID NO: 28 - protein, 15 aa, synthetic construct
RASKSVSTSSYSYLA

SEQ ID NO: 29 - protein, 15 aa, synthetic construct
RASKTVSTSSYSYMH

SEQ ID NO: 30 - protein, 7 aa, synthetic construct
YASYLQS

SEQ ID NO: 31 - protein, 17 aa, synthetic construct
  VARIANT 5: note = X can be any amino acid
  VARIANT 14: note = X can be any amino acid
YISSXSSTIYYADXVKG

SEQ ID NO: 32 - protein, 15 aa, synthetic construct
  VARIANT 5: note = X can be any amino acid
  VARIANT 14..15: note = X can be any amino acid
RASKXVSTSSYSYXX

SEQ ID NO: 33 - protein, 7 aa, synthetic construct
  VARIANT 6: note = X can be any amino acid
YASYLXS

SEQ ID NO: 34 - protein, 5 aa, synthetic construct
QYGMH

SEQ ID NO: 35 - protein, 5 aa, synthetic construct
EYGMH

SEQ ID NO: 36 - protein, 5 aa, synthetic construct
  VARIANT 5: note = K can be replaced by R
BYGMK

SEQ ID NO: 37 - protein, 5 aa, synthetic construct
  VARIANT 5: note = K can be replaced by R
QYGMK

SEQ ID NO: 38 - protein, 5 aa, synthetic construct
  VARIANT 5: note = K can be replaced by R
EYGMK

SEQ ID NO: 39 - protein, 17 aa, synthetic construct
YISSGSSTIYYAETVKG

SEQ ID NO: 40 - protein, 17 aa, synthetic construct
  VARIANT 16: note = R can be replaced by H
YISSGSSTIYYADTVRG

SEQ ID NO: 41 - protein, 17 aa, synthetic construct
  VARIANT 16: note = R can be replaced by H
YISSGSSTIYYAETVRG

SEQ ID NO: 42 - protein, 7 aa, synthetic construct
  VARIANT 1: note = X can be any amino acid
XGLLLDY

SEQ ID NO: 43 - protein, 7 aa, synthetic construct
RGLLLEY

SEQ ID NO: 44 - protein, 7 aa, synthetic construct
  VARIANT 1: note = K can be replaced by H
KGLLLEY

SEQ ID NO: 45 - protein, 15 aa, synthetic construct
  VARIANT 1: note = K can be replaced by H
KASKSVSTSSYSYMH

SEQ ID NO: 46 - protein, 15 aa, synthetic construct
  VARIANT 4: note = R can be replaced by H
RASRSVSTSSYSYMH

SEQ ID NO: 47 - protein, 15 aa, synthetic construct
  VARIANT 15: note = K can be replaced by R
RASKSVSTSSYSYMK

SEQ ID NO: 48 - protein, 15 aa, synthetic construct
  VARIANT 1: note = K can be replaced by H
  VARIANT 4: note = R can be replaced by H
KASRSVSTSSYSYMH

SEQ ID NO: 49 - protein, 15 aa, synthetic construct
  VARIANT 1: note = K can be replaced by H
  VARIANT 15: note = K can be replaced by R
KASKSVSTSSYSYMK

SEQ ID NO: 50 - protein, 15 aa, synthetic construct
  VARIANT 4: note = R can be replaced by H
  VARIANT 15: note = K can be replaced by R
RASRSVSTSSYSYMK

SEQ ID NO: 51 - protein, 7 aa, synthetic construct
YASYLDS

SEQ ID NO: 52 - protein, 9 aa, synthetic construct
  VARIANT 2: note = K can be replaced by R
QKSREFPWT

SEQ ID NO: 53 - protein, 9 aa, synthetic construct
  VARIANT 4: note = K can be replaced by H
QHSKEFPWT

SEQ ID NO: 54 - protein, 9 aa, synthetic construct
QHSRDFPWT

SEQ ID NO: 55 - protein, 9 aa, synthetic construct
  VARIANT 2: note = K can be replaced by R
  VARIANT 4: note = K can be replaced by H
QKSKEFPWT

SEQ ID NO: 56 - protein, 9 aa, synthetic construct
  VARIANT 2: note = K can be replaced by R
QKSRDFPWT

SEQ ID NO: 57 - protein, 9 aa, synthetic construct
  VARIANT 4: note = K can be replaced by H
QHSKDFPWT

SEQ ID NO: 58 - protein, 5 aa, synthetic construct
  VARIANT 1: note = D can be replaced by E, N, Q, R, or K
  VARIANT 5: note = K can be replaced by R or H
DYGMK

SEQ ID NO: 59 - protein, 17 aa, synthetic construct
  VARIANT 5: note = G can be replaced by S
  VARIANT 13: note = D can be replaced by E
  VARIANT 14: note = T can be replaced by S
  VARIANT 16: note = K can be replaced by R or H
YISSGSSTIYYADTVKG

SEQ ID NO: 60 - protein, 7 aa, synthetic construct
  VARIANT 1: note = K can be replaced by R or H
  VARIANT 6: note = K can be replaced by E
KGLLLDY

SEQ ID NO: 61 - protein, 15 aa, synthetic construct
  VARIANT 1: note = K can be replaced by R or H
  VARIANT 4: note = K can be replaced by R or H
  VARIANT 5: note = T can be replaced by S
  VARIANT 14: note = M can be replaced by L
  VARIANT 15: note = K can be replaced by R, H or A
KASKTVSTSSYSYMK

SEQ ID NO: 62 - protein, 7 aa, synthetic construct
  VARIANT 6: note = D can be replaced by E, N or Q
YASYLDS

SEQ ID NO: 63 - protein, 9 aa, synthetic construct
  VARIANT 2: note = K can be replaced by R or H
  VARIANT 4: note = K can be replaced by R or H
  VARIANT 5: note = D can be replaced by E
QKSKDFPWT

SEQ ID NO: 64 - protein, 116 aa, synthetic construct
EVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSS

SEQ ID NO: 65 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSS

SEQ ID NO: 66 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSS

SEQ ID NO: 67 - protein, 116 aa, synthetic construct
EVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSS

SEQ ID NO: 68 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSS

SEQ ID NO: 69 - protein, 116 aa, synthetic construct
EVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSS

SEQ ID NO: 70 - protein, 116 aa, synthetic construct
EVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSS

SEQ ID NO: 71 - protein, 446 aa, synthetic construct
EVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 446 aa, synthetic construct
EVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 446 aa, synthetic construct
EVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 446 aa, synthetic construct
EVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 465 aa, synthetic construct
MGWSCIILFLVATATGVHSEVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 465 aa, synthetic construct
MGWSCIILFLVATATGVHSEVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 465 aa, synthetic construct
MGWSCIILFLVATATGVHSEVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 465 aa, synthetic construct
MGWSCIILFLVATATGVHSEVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 111 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIK

SEQ ID NO: 86 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIK

SEQ ID NO: 87 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIK

SEQ ID NO: 88 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIK

SEQ ID NO: 89 - protein, 111 aa, synthetic construct
DIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIK

SEQ ID NO: 90 - protein, 111 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIK

SEQ ID NO: 91 - protein, 218 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 218 aa, synthetic construct
DIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 218 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 237 aa, synthetic construct
MGWSCIILFLVATATGVHSDIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 237 aa, synthetic construct
MGWSCIILFLVATATGVHSDIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 31 aa, synthetic construct
RASKSVSTSSYSYMHWYQQKPGQPPKLLIKY

SEQ ID NO: 104 - protein, 31 aa, synthetic construct
RASKTVSTSSYSYMHWYQQKPGQPPKLLIKY

SEQ ID NO: 105 - protein, 33 aa, synthetic construct
RVTITCRASKSVSTSSYSYMHWYQQKPGKAPKL

SEQ ID NO: 106 - protein, 32 aa, synthetic construct
  VARIANT 1: note = X can be any amino acid such as a basic residue (R or K)
  VARIANT 30: note = X can be any amino acid such as a basic residue (R or K)
XRASKTVSTSSYSYMHWYQQKPGQPPKLLXKY

SEQ ID NO: 107 - protein, 7 aa, synthetic construct
GQSSRSS

SEQ ID NO: 108 - protein, 18 aa, synthetic construct
GQSSRSSSGGGSSGGGGS

SEQ ID NO: 109 - protein, 4 aa, synthetic construct
GSGS

SEQ ID NO: 110 - protein, 4 aa, synthetic construct
GGGS

SEQ ID NO: 111 - protein, 10 aa, synthetic construct
GGGGSGGGGS

SEQ ID NO: 112 - protein, 20 aa, synthetic construct
GGGGSGGGGSGGGGSGGGGS

SEQ ID NO: 113 - protein, 15 aa, synthetic construct
GGGGSGGGGSGGGGS

SEQ ID NO: 114 - protein, 21 aa, synthetic construct
RADAAPGGGGSGGGGSGGGGS

SEQ ID NO: 115 - protein, 19 aa, synthetic construct
ASTKGPSVFPLAPLESSGS

SEQ ID NO: 116 - protein, 8 aa, synthetic construct
GFTFSDYG

SEQ ID NO: 117 - protein, 8 aa, synthetic construct
GFTFSNYG

SEQ ID NO: 118 - protein, 8 aa, synthetic construct
ISSGSSTI

SEQ ID NO: 119 - protein, 8 aa, synthetic construct
ISSSSSTI

SEQ ID NO: 120 - protein, 9 aa, synthetic construct
ARRGLLLDY

SEQ ID NO: 121 - protein, 10 aa, synthetic construct
KSVSTSSYSY

SEQ ID NO: 122 - protein, 10 aa, synthetic construct
KTVSTSSYSY

SEQ ID NO: 123 - protein, 9 aa, synthetic construct
QHSREFPWT

SEQ ID NO: 124 - protein, 108 aa, synthetic construct
MTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 125 - protein, 116 aa, synthetic construct
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 126 - protein, 111 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 127 - protein, 116 aa, synthetic construct; identical to SEQ ID NO: 125

SEQ ID NO: 128-137 - skipped (10 entries)

SEQ ID NO: 138 - protein, 4 aa, synthetic construct
GGFG

SEQ ID NO: 139 - protein, 4 aa, synthetic construct
GFGG

SEQ ID NO: 140 - protein, 4 aa, synthetic construct
GGGF

SEQ ID NO: 141 - protein, 4 aa, synthetic construct
FGGG

SEQ ID NO: 142 - protein, 4 aa, synthetic construct
GGGG

SEQ ID NO: 143 - protein, 4 aa, synthetic construct
LLQG

SEQ ID NO: 144 - protein, 116 aa, synthetic construct
  VARIANT 13: note = X can be any amino acid
  VARIANT 42: note = X can be any amino acid
  VARIANT 84: note = X can be any amino acid
  VARIANT 88: note = X can be any amino acid
  VARIANT 93: note = X can be any amino acid
  VARIANT 111..112: note = X can be any amino acid
  VARIANT 116: note = X can be any amino acid
EVQLVESGGGLVXPGGSRKLSCAASGFTFSNYGMHWVRQAPXKGLEWVAYISSGSSTIYYADTVKGRFTISRDNAKNTLFLQMXSLRXEDTAXYYCARRGLLLDYWGQGTXXTVSX

SEQ ID NO: 145 - protein, 116 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSA

SEQ ID NO: 146 - protein, 111 aa, synthetic construct
  VARIANT 15: note = X can be any amino acid
  VARIANT 87: note = X can be any amino acid
  VARIANT 89: note = X can be any amino acid
  VARIANT 108: note = X can be any amino acid
DIVLTQSPASLAVSXGQRATISCRASKSVSTSSYSYMHWYQQKPGQPPKLLIKYASYLESGVPARFSGSGSGTDFTLNIHPVEEEDXAXYYCOHSREFPWTFGGGTKXEIK

SEQ ID NO: 147 - protein, 111 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCOHSREFPWTFGGGTKLEIK

SEQ ID NO: 148 - protein, 111 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCOHSREFPWTFGGGTKVEIK

SEQ ID NO: 149 - protein, 5 aa, synthetic construct
KVEPK

(19) United States
(12) Patent Application Publication
(10) Pub. No.: US 2026/0207769 A1
(43) Pub. Date: Jul. 23, 2026

(54) ANTIBODY DRUG CONJUGATES

(71) Applicants: YALE UNIVERSITY, New Haven, CT (US); Gennao Bio, Inc., Pennington, NJ (US)

(72) Inventors: Peter GLAZER, Guilford, CT (US); Elias QUIJANO, Durham, CT (US); Ravi CHARI, Needham, MA (US); Dale LUDWIG, Pennington, NJ (US); Venugopalareddy BOMMIREDDYVENKATA, Pennington, NJ (US)

(21) Appl. No.: 19/144,384

(22) PCT Filed: Dec. 27, 2023
(86) PCT No.: PCT/US2023/086103

§ 371 Date: Jun. 27, 2025

Related U.S. Application Data
(60) Provisional application No. 63/477,364, filed on Dec. 27, 2022.
(60) Provisional application No. 63/513,065, filed on Jul. 11, 2023.
(60) Provisional application No. 63/580,921, filed on Sep. 6, 2023.
(60) Provisional application No. 63/585,915, filed on Sep. 27, 2023.

(51) Int. Cl. A61K 47/68; A61P 35/00

(52) U.S. Cl. CPC A61K 47/6889; A61K 47/6803; A61K 47/68031; A61K 47/68033; A61K 47/68035; A61K 47/68037; A61K 47/6809; A61K 47/6811; A61P 35/00

(57) ABSTRACT

The present disclosure provides an antibody-drug conjugate (ADC) having an antibody, antigen-binding fragment thereof, or cell-penetrating fragment thereof; a linker; and a payload moiety. Compositions including the ADC and methods of using the ADC are also provided.

### CROSS REFERENCE TO RELATED APPLICATIONS

This application claim priority to U.S. Provisional Patent Application No. 63/477,364, filed Dec. 27, 2022, U.S. Provisional Patent Application No. 63/513,065, filed Jul. 11, 2023, U.S. Provisional Patent Application No. 63/580,921, filed Sep. 6, 2023, and U.S. Provisional Patent Application No. 63/585,915, filed Sep. 27, 2023, the contents of which are hereby incorporated by reference herein, in their entireties, for all purposes.

### STATEMENT REGARDING FEDERALLY-SPONSORED RESEARCH AND DEVELOPMENT

This invention was made with government support under R35 CA197574, awarded by National Institutes of Health. The Government has certain rights in the invention.

### BACKGROUND

Antibody-drug conjugates (ADCs), a rapidly growing class of targeted therapeutics, represent a promising new approach to improving the selectivity and cytotoxic activity of drugs. These therapeutic agents are comprised of an antibody (or antibody fragment) that is covalently linked to a payload drug to form an immunoconjugate. The antibody directs the ADC to bind to the targeted cell. Upon entry into the cell, the ADC can localize in and around the cell nucleus and release its payload, which treats or kills the cell.

The monoclonal 3E10 antibody (“3E10”), has been developed as a molecular delivery vehicle that transports a variety of biologically important molecules into target cells, and 3E10 has been shown to preferentially target cancer cells. 3E10 penetrates cells in an Fc-independent mechanism, as evidenced by the ability of 3E10 fragments lacking an Fc to penetrate cells, that involves the presence of the nucleoside transporter ENT2 (Weisbart et al., Scientific Reports volume 5, Article number: 12022 (2015), Zack et al., J Immunol 157, 2082-2088 (1996), Hansen et al., J Biol Chem 282, 20790-20793 (2007)). A 3E10 single chain variable fragment, 3E10 scFv has previously been shown to be capable of penetrating living cells and nuclei in an ENT2-dependent manner, with efficiency of uptake impaired in ENT2-deficient cells (Hansen, et al., J. Biol. Chem. 282, 20790-20793 (2007)).

3E10 has not shown any cellular toxicity in vitro or in vivo in studies to date. In contrast, several antibodies that penetrate living cells are frequently toxic or injurious and can trigger some of the pathologic manifestations of the autoimmune diseases in which they are found. The 3E10 antibody is an ideal molecular delivery vehicle due to its efficiency in penetrating living cells with specific nuclear localization, absence of toxicity, and successful delivery of therapeutic cargo proteins in vitro and in vivo.

Currently, most ADCs are designed to target chemotherapeutic warheads to specific cancer types based on expression of a cell surface antigen. ADCs designed to address breast cancer, for instance, are often designed to target HER2, a cell surface receptor often expressed in breast cancer cells. There is a need for new ADCs for improved delivery of drugs to the targeted cell, including new ADCs having the ability to deliver drugs to multiple types of cancer cells.

### SUMMARY

In embodiments, the disclosure provides a conjugate of Formula (I):

A-(L-Pᵣ)_{q} Formula (I),
wherein in Formula (I):
   A is an antibody, antigen-binding fragment thereof or cell-penetrating fragment thereof comprising a heavy chain variable region (VH) CDR1 comprising the amino acid sequence of SEQ ID NO:58, CDR2 comprising the amino acid sequence of SEQ ID NO:59, CDR3 comprising SEQ ID NO:60; and a light chain variable region (VL) CDR1 comprising the amino acid sequence of SEQ ID NO:61, CDR2 comprising the amino acid sequence of SEQ ID NO:62, CDR3 comprising the amino acid sequence of SEQ ID NO:63;
   L is a linker;
   P is a payload moiety;
   r is an integer from 1 to 4; and
   q is an integer from 1 to 16.

In some embodiments, the linker L comprises one or more groups selected from optionally substituted alkylene, optionally substituted alkenylene, optionally substituted alkynylene, optionally substituted arylene, optionally substituted cycloalkylene, optionally substituted heteroalkylene, optionally substituted heteroarylene, optionally substituted heterocycloalkylene, —NR^{a}— (Linker ID NO. 1), —N═CR^{a}— (Linker ID NO. 2), —CR^{a}═N— (Linker ID NO. 3), —S— (Linker ID NO. 4), —S(O)— (Linker ID NO. 5), —S(O)₂— (Linker ID NO. 6), —OP(O)OR^{a}— (Linker ID NO. 7), —OP(O)OR^{a}O— (Linker ID NO. 8), —P(O)OR^{a}O— (Linker ID NO. 9), —O— (Linker ID NO. 10), —CR^{b}₂— (Linker ID NO. 11), —[(CR^{b}₂)₁₋₁₂O]₁₋₅₀— (Linker ID NO. 12), —C(O)— (Linker ID NO. 13), —C(S)— (Linker ID NO. 14), —C(═N—OH)— (Linker ID NO. 15), —C(NR^{a})— (Linker ID NO. 16), —C(NH₂Cl)— (Linker ID NO. 17), —C(O)O— (Linker ID NO. 18), —OC(O)— (Linker ID NO. 19), —C(O)S— (Linker ID NO. 20), —SC(O)— (Linker ID NO. 21), —C(O)NR^{a}— (Linker ID NO. 22), —NR^{a}C(O)— (Linker ID NO. 23), —C(O)NR^{a}SO₂— (Linker ID NO. 24), —SO₂NR^{a}C(O)— (Linker ID NO. 25), —OC(O)O— (Linker ID NO. 26), —OC(O)S— (Linker ID NO. 27), —SC(O)O— (Linker ID NO. 28), —OC(O)NR^{a}— (Linker ID NO. 29), —NR^{a}C(O)O— (Linker ID NO. 30), —SC(O)NR^{a}— (Linker ID NO. 31), —NR^{a}C(O)S— (Linker ID NO. 32), —S(O)ₜN(R^{a})— (where t is 1 or 2) (Linker ID NO. 33), —N(R^{a})S(O)ₜ— (where t is 1 or 2) (Linker ID NO. 34), and —X_{AA}— (Linker ID NO. 35);
each R^{a} is independently selected at each occurrence from hydrogen, optionally substituted alkyl, optionally substituted fluoroalkyl, optionally substituted cycloalkyl, optionally substituted cycloalkylalkyl, optionally substituted alkenyl, optionally substituted fluoroalkenyl, optionally substituted cycloalkenyl, optionally substituted cycloalkenylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted alkenyl, optionally substituted fluoroalkenyl, optionally substituted cycloalkenyl, optionally substituted cycloalkenylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
two independent R^{b} groups or an R^{a} and an R^{b} can be joined together to form an optionally substituted cycle; and
—X_{AA}— is an amino acid sequence comprising 1 to 6 amino acid moieties.

In some embodiments, each amino acid moiety of —X_{AA}— is independently selected from alanine (Ala), arginine (Arg), asparagine (Asn), aspartic acid (Asp), cysteine (Cys), glutamine (Gin), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), valine (Val), citrulline (Cit), and homocitrulline (HoCit). In some embodiments, the linker L comprises one or more groups selected from optionally substituted C₁-C₁₈ alkylene (Linker ID NO. 36), —C≡C— (Linker ID NO. 339), —CR^{a}═CR^{a}— (Linker ID NO. 38), optionally substituted 6- to 14-membered arylene (Linker ID NO. 39), optionally substituted C₃-C₂₀ cycloalkylene (Linker ID NO. 40), —[CH₂O]₁₋₁₈— (Linker ID NO. 41), —[CH₂CH₂O]₁₋₁₈— (Linker ID NO. 42), —[CH₂CH₂CH₂O]₁₋₁₈— (Linker ID NO. 43), optionally substituted 5- to 18-membered heteroarylene (Linker ID NO. 44), optionally substituted 3- to 20-membered heterocycloalkylene (Linker ID NO. 45), —NR^{a}— (Linker ID NO. 1), —N═CR^{a}— (Linker ID NO. 2), —CR^{a}═N— (Linker ID NO. 3), —S— (Linker ID NO. 4), —S(O)— (Linker ID NO. 5), —S(O)₂— (Linker ID NO. 6), —OP(O)OR^{a}— (Linker ID NO. 7), —OP(O)OR^{a}O— (Linker ID NO. 8), —P(O)OR^{a}O— (Linker ID NO. 9), —O— (Linker ID NO. 10), —CR^{b}₂— (Linker ID NO. 11), —[(CR^{b}₂)₁₋₁₂O]₁₋₅₀— (Linker ID NO. 12), —C(O)— (Linker ID NO. 13), —C(S)— (Linker ID NO. 14), —C(═N—OH)— (Linker ID NO. 15), —C(NR^{a})— (Linker ID NO. 16), —C(NH₂Cl)— (Linker ID NO. 17), —C(O)O— (Linker ID NO. 18), —OC(O)— (Linker ID NO. 19), —C(O)S— (Linker ID NO. 20), —SC(O)— (Linker ID NO. 21), —C(O)NR^{a}— (Linker ID NO. 22), —NR^{a}C(O)— (Linker ID NO. 23), —C(O)NR^{a}SO₂— (Linker ID NO. 24), —SO₂NR^{a}C(O)— (Linker ID NO. 25), —OC(O)O— (Linker ID NO. 26), —OC(O)S— (Linker ID NO. 27), —SC(O)O— (Linker ID NO. 28), —OC(O)NR^{a}— (Linker ID NO. 29), —NR^{a}C(O)O— (Linker ID NO. 30), —SC(O)NR^{a}— (Linker ID NO. 31), —NR^{a}C(O)S— (Linker ID NO. 32), —S(O)ₜN(R^{a})— (where t is 1 or 2) (Linker ID NO. 33), —N(R^{a})S(O)ₜ— (where t is 1 or 2) (Linker ID NO. 34), and —X_{AA}— (Linker ID NO. 35).

In some embodiments, the linker L comprises one or more groups selected from optionally substituted C₁-C₁₆ alkylene (Linker ID NO. 46), —C≡C— (Linker ID NO. 339), —CR^{a}═CR^{a}— (Linker ID NO. 38), optionally substituted phenylene (Linker ID NO. 47), optionally substituted C₃-C₆ cycloalkylene (Linker ID NO. 48), —[CH₂CH₂O]₁₋₁₆— (Linker ID NO. 49), —[CH₂CH₂CH₂O]₁₋₁₆— (Linker ID NO. 50), optionally substituted 5- to 6-membered heteroarylene (Linker ID NO. 51), optionally substituted 5- to 20-membered heterocycloalkylene (Linker ID NO. 52), —NR^{a}— (Linker ID NO. 1), —N═CR^{a}— (Linker ID NO. 2), —CR^{a}═N— (Linker ID NO. 3), —S— (Linker ID NO. 4), —OP(O)OR^{a}O— (Linker ID NO. 8), —O— (Linker ID NO. 10), —CR^{b}₂— (Linker ID NO. 11), —C(O)— (Linker ID NO. 13), —C(O)O— (Linker ID NO. 18), —OC(O)— (Linker ID NO. 19), —C(O)NR^{a}— (Linker ID NO. 22), —NR^{a}C(O)— (Linker ID NO. 23), —OC(O)O— (Linker ID NO. 26), —OC(O)NR^{a}— (Linker ID NO. 29), —NR^{a}C(O)O— (Linker ID NO. 30), and —X_{AA}— (Linker ID NO. 35).

In some embodiments, the linker L comprises one or more groups selected from —[C(R^{b})₂]₁₋₁₆— (Linker ID NO. 341), —C≡C— (Linker ID NO. 339), —CR^{a}═CR^{a}— (Linker ID NO. 38), —[CH₂CH₂O]₁₋₁₆— (Linker ID NO. 49), —NR^{a}— (Linker ID NO. 1), —N═CR^{a}— (Linker ID NO. 2), —CR^{a}═N— (Linker ID NO. 3), —S— (Linker ID NO. 4), —OP(O)OR^{a}O— (Linker ID NO. 7), —O— (Linker ID NO. 10), —C(O)— (Linker ID NO. 13), —C(O)O— (Linker ID NO. 18), —OC(O)— (Linker ID NO. 19), —C(O)NR^{a}— (Linker ID NO. 22), —NR^{a}C(O)— (Linker ID NO. 23), —OC(O)O— (Linker ID NO. 26), —OC(O)NR^{a}— (Linker ID NO. 29), —NR^{a}C(O)O— (Linker ID NO. 30), —X_{AA}— (Linker ID NO. 35),

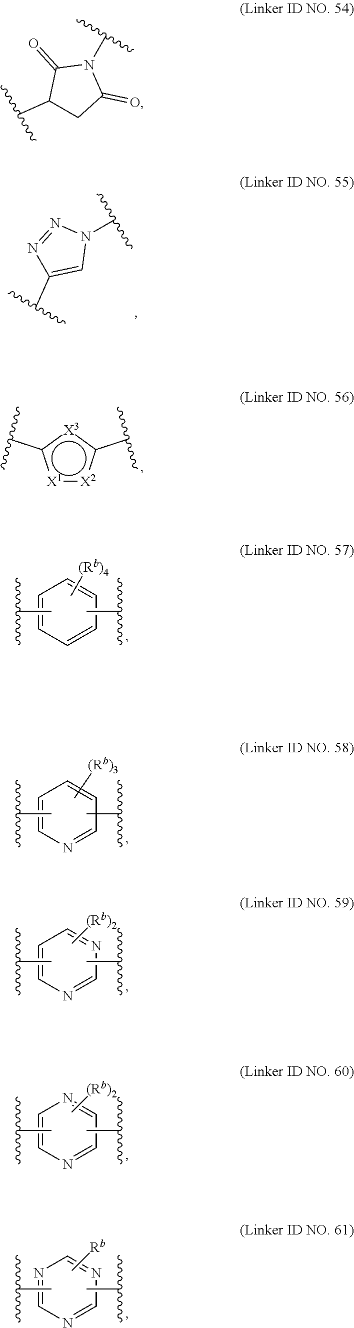

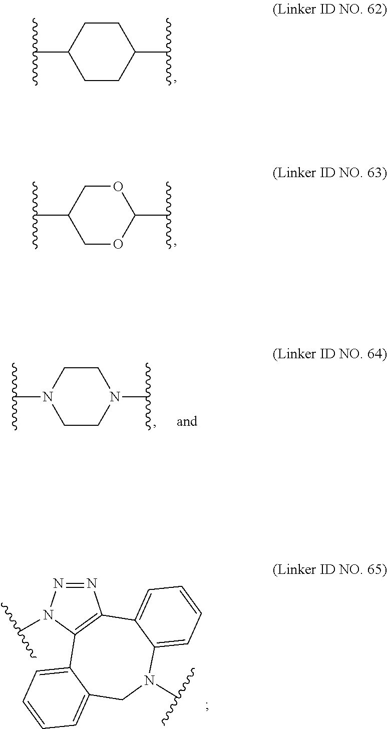 [SMILES: *C1CCC(*)CC1 ; *C1COC(*)OC1 ; *N1CCN(*)CC1 ; *N1Cc2ccccc2-c2c(nnn2*)-c2ccccc21]

and
X¹, X², and X³ are independently selected at each occurrence from NR^{a}, N, CR^{b}, S, and O.

In some embodiments, the linker L is of Formula (L-1):

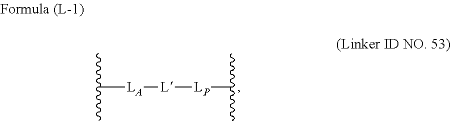

wherein in Formula (L-1):
   L^{A} is a connecting moiety through which A is covalently attached to L′;
   L′ is a bond or comprises one or more groups selected from optionally substituted alkylene, optionally substituted alkenylene, optionally substituted alkynylene, optionally substituted arylene, optionally substituted cycloalkylene, optionally substituted heteroalkylene, optionally substituted heteroarylene, optionally substituted heterocycloalkylene, —NR^{a}— (Linker ID NO. 1), —N═CR^{a}— (Linker ID NO. 2), —CR^{a}═N— (Linker ID NO. 3), —S— (Linker ID NO. 4), —S(O)— (Linker ID NO. 5), —S(O)₂— (Linker ID NO. 6), —OP(O)OR^{a}— (Linker ID NO. 7), —OP(O)OR^{a}O— (Linker ID NO. 8), —P(O)OR^{a}O— (Linker ID NO. 9), —O— (Linker ID NO. 10), —CR^{b}₂— (Linker ID NO. 11), —[(CR^{b}₂)₁₋₁₂O]₁₋₅₀— (Linker ID NO. 12), —C(O)— (Linker ID NO. 13), —C(S)— (Linker ID NO. 14), —C(═N—OH)— (Linker ID NO. 15), —C(NR^{a})— (Linker ID NO. 16), —C(NH₂Cl)— (Linker ID NO. 17), —C(O)O— (Linker ID NO. 18), —OC(O)— (Linker ID NO. 19), —C(O)S— (Linker ID NO. 20), —SC(O)— (Linker ID NO. 21), —C(O)NR^{a}— (Linker ID NO. 22), —NR^{a}C(O)— (Linker ID NO. 23), —C(O)NR^{a}SO₂— (Linker ID NO. 24), —SO₂NR^{a}C(O)— (Linker ID NO. 25), —OC(O)O— (Linker ID NO. 26), —OC(O)S— (Linker ID NO. 27), —SC(O)O— (Linker ID NO. 28), —OC(O)NR^{a}— (Linker ID NO. 29), —NR^{a}C(O)O— (Linker ID NO. 30), —SC(O)NR^{a}— (Linker ID NO. 31), —NR^{a}C(O)S— (Linker ID NO. 32), —S(O)ₜN(R^{a})— (where t is 1 or 2) (Linker ID NO. 33), —N(R^{a})S(O)ₜ— (where t is 1 or 2) (Linker ID NO. 34), and —X_{AA}— (Linker ID NO. 35); and
   L_{P} is a connecting moiety through which P is covalently attached to L′.

In some embodiments, the linker L comprises at least one cleavable moiety. In some embodiments, the cleavable moiety comprises an acid-labile moiety, a reducibly-labile moiety, or an enzymatically-labile moiety. In some embodiments, the cleavable moiety comprises one or more groups selected from:

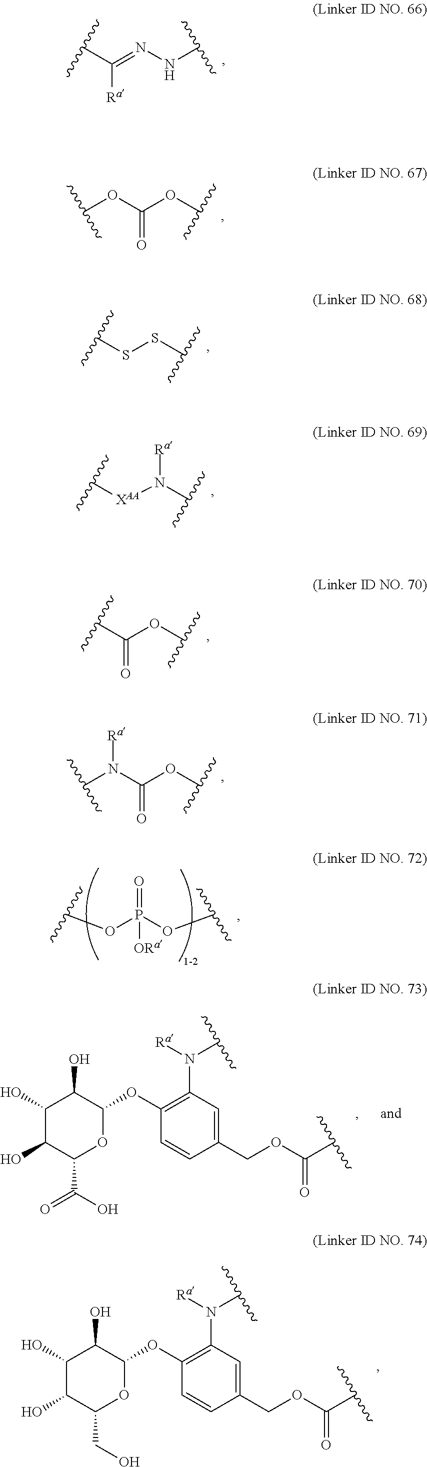

wherein:
   each R^{a′} is independently selected at each occurrence from hydrogen, optionally substituted alkyl, and optionally substituted heteroalkyl.

In some embodiments, the cleavable moiety comprises the reducibly-labile moiety —S—S— (Linker ID NO. 340).

In some embodiments, the linker L is of formula (L-10):

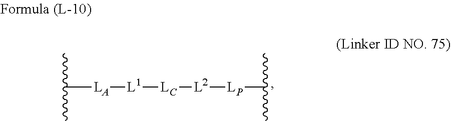

wherein in Formula (L-10):
   L_{A} is selected from a bond, —NR^{a′}—, and —S—;
   L¹ is a bond or comprises one or more groups selected from optionally substituted C₁-C₁₈ alkylene (Linker ID NO. 36), —C≡C— (Linker ID NO. 339), —CR^{a}═CR^{a} (Linker ID NO. 38)-, optionally substituted 6- to 14-membered arylene (Linker ID NO. 39), optionally substituted C₃-C₂₀ cycloalkylene (Linker ID NO. 40), —[CH₂O]₁₋₁₈— (Linker ID NO. 41), —[CH₂CH₂O]₁₋₁₈— (Linker ID NO. 42), —[CH₂CH₂CH₂O]₁₋₁₈— (Linker ID NO. 43), optionally substituted 5- to 18-membered heteroarylene (Linker ID NO. 44), optionally substituted 3- to 20-membered heterocycloalkylene (Linker ID NO. 45), —NR^{a}— (Linker ID NO. 1), —N═CR^{a}— (Linker ID NO. 2), —CR^{a}═N— (Linker ID NO. 3), —S— (Linker ID NO. 4), —OP(O)OR^{a}O— (Linker ID NO. 8), —O— (Linker ID NO. 10), —CR^{b}₂— (Linker ID NO. 11), —C(O)— (Linker ID NO. 13), —C(O)O— (Linker ID NO. 18), —OC(O)— (Linker ID NO. 19), —C(O)S— (Linker ID NO. 20), —SC(O)— (Linker ID NO. 21), —C(O)NR^{a} (Linker ID NO. 22)-, —NR^{a}C(O)— (Linker ID NO. 23), —OC(O)O— (Linker ID NO. 26), —OC(O)S— (Linker ID NO. 27), —SC(O)O— (Linker ID NO. 28), —OC(O)NR^{a}— (Linker ID NO. 29), —NR^{a}C(O)O— (Linker ID NO. 30), —SC(O)NR^{a}— (Linker ID NO. 31), —NR^{a}C(O)S— (Linker ID NO. 32), and —X_{AA}— (Linker ID NO. 35);
   L_{C} is selected from an acid-labile moiety, a reducibly-labile moiety, and an enzymatically-labile moiety;
   L² is a bond or comprises one or more groups selected from optionally substituted C₁-C₁₈ alkylene (Linker ID NO. 36), —C≡C— (Linker ID NO. 339), —CR^{a}═CR^{a} (Linker ID NO. 38)-, optionally substituted 6- to 14-membered arylene (Linker ID NO. 39), optionally substituted C₃-C₂₀ cycloalkylene (Linker ID NO. 40), —[CH₂O]₁₋₁₈— (Linker ID NO. 41), —[CH₂CH₂O]₁₋₁₈— (Linker ID NO. 42), —[CH₂CH₂CH₂O]₁₋₁₈— (Linker ID NO. 43), optionally substituted 5- to 18-membered heteroarylene (Linker ID NO. 44), optionally substituted 3- to 20-membered heterocycloalkylene (Linker ID NO. 45), —NR^{a}— (Linker ID NO. 1), —N═CR^{a}— (Linker ID NO. 2), —CR^{a}═N— (Linker ID NO. 3), —S— (Linker ID NO. 4), —OP(O)OR^{a}O— (Linker ID NO. 8), —O— (Linker ID NO. 10), —CR^{b}₂— (Linker ID NO. 11), —C(O)— (Linker ID NO. 13), —C(O)O— (Linker ID NO. 18), —OC(O)— (Linker ID NO. 19), —C(O)S— (Linker ID NO. 20), —SC(O)— (Linker ID NO. 21), —C(O)NR^{a} (Linker ID NO. 22)-, —NR^{a}C(O)— (Linker ID NO. 23), —OC(O)O— (Linker ID NO. 26), —OC(O)S— (Linker ID NO. 27), —SC(O)O— (Linker ID NO. 28), —OC(O)NR^{a}— (Linker ID NO. 29), —NR^{a}C(O)O— (Linker ID NO. 30), —SC(O)NR^{a}— (Linker ID NO. 31), —NR^{a}C(O)S— (Linker ID NO. 32), and —X_{AA}— (Linker ID NO. 35);
   L^{P} is selected from a bond, —NR^{a′}— (Linker ID NO. 1), —S— (Linker ID NO. 4), and —O— (Linker ID NO. 10);
   each R^{a} is independently selected at each occurrence from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
   each R^{a′} is independently selected at each occurrence from hydrogen, optionally substituted alkyl, and optionally substituted heteroalkyl;
   each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or two independent R^{b} groups are taken together to form optionally substituted cycloalkyl, optionally substituted aryl, optionally substituted heterocycloalkyl, or optionally substituted heteroaryl; and
   —X_{AA}— is an amino acid sequence comprising 1 to 4 amino acid moieties.

In some embodiments, L_{C} is selected from:

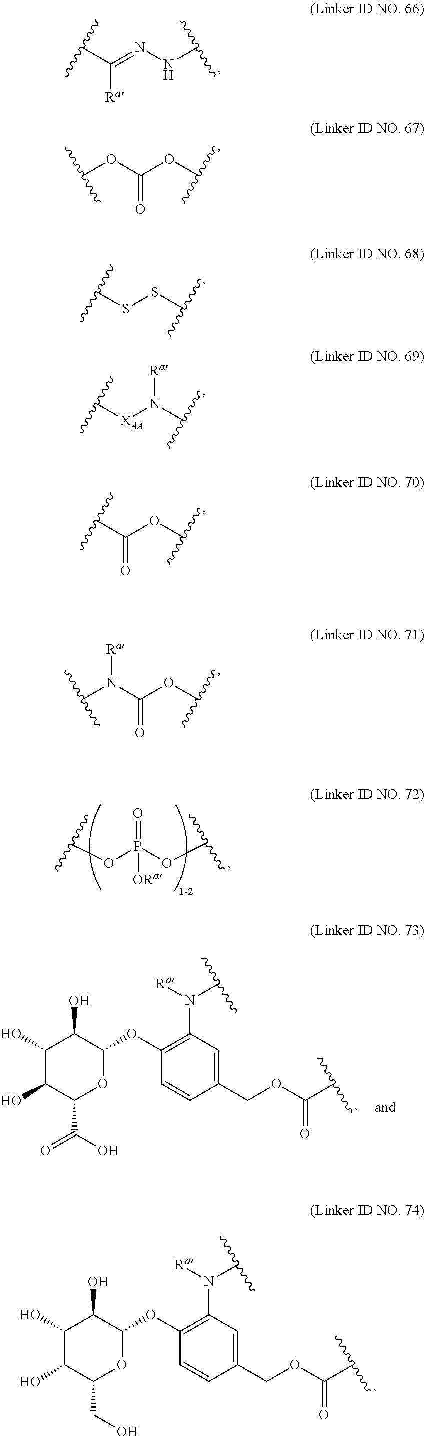

wherein:
   each R^{a′} is independently selected at each occurrence from hydrogen, optionally substituted alkyl, and optionally substituted heteroalkyl.

In some embodiments, L_{C} is —S—S— (Linker ID NO. 340).

In some embodiments, the linker L is of Formula (L-11):

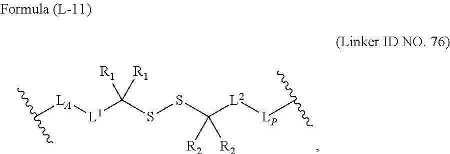

wherein in Formula (L-11):
   L_{A} is selected from a bond, —NH—, and —S—;
   L¹ is a bond or comprises one or more groups selected from —[C(R^{b})₂]₁₋₁₆— (Linker ID NO. 341), —C≡C— (Linker ID NO. 339), —CR^{a}═CR^{a}— (Linker ID NO. 38), —[CH₂CH₂O]₁₋₁₆— (Linker ID NO. 49), —NR^{a}— (Linker ID NO. 1), —N═CR^{a}— (Linker ID NO. 2), —CR^{a}═N— (Linker ID NO. 3), —S— (Linker ID NO. 4), —OP(O)OR O— (Linker ID NO. 7), —O— (Linker ID NO. 10), —C(O)— (Linker ID NO. 13), —C(O)O— (Linker ID NO. 18), —OC(O)— (Linker ID NO. 19), —C(O)NR^{a}— (Linker ID NO. 22), —NR^{a}C(O)— (Linker ID NO. 23), —OC(O)O— (Linker ID NO. 26), —X_{AA}— (Linker ID NO. 35), —OC(O)NR^{a}— (Linker ID NO. 29), —NR^{a}C(O)O— (Linker ID NO.

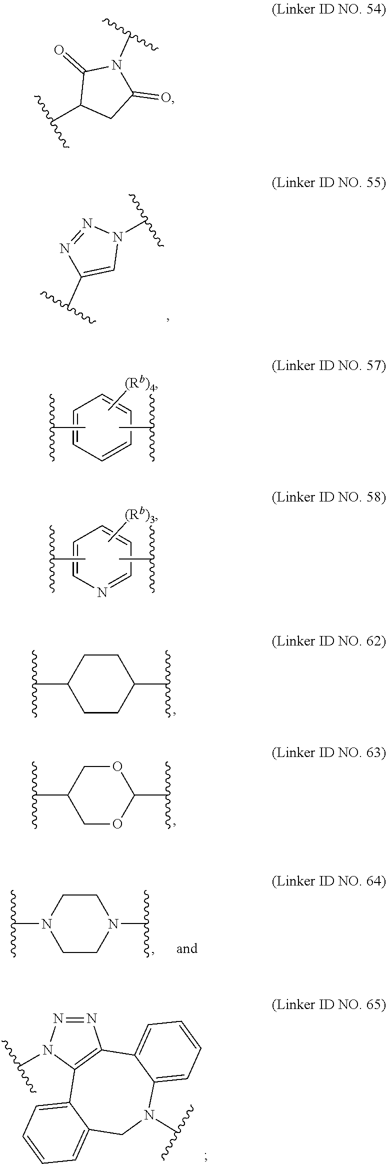

L² is a bond or comprises one or more groups selected from —[C(R^{b})₂]₁₋₁₆— (Linker ID NO. 341), —C≡C— (Linker ID NO. 339), —CR^{a}═CR^{a}— (Linker ID NO. 38), —[CH₂CH₂O]₁₋₁₆— (Linker ID NO. 49), —NR^{a}— (Linker ID NO. 1), —N═CR^{a}— (Linker ID NO. 2), —CR^{a}═N— (Linker ID NO. 3), —S— (Linker ID NO. 4), —OP(O)OR O— (Linker ID NO. 7), —O— (Linker ID NO. 10), —C(O)— (Linker ID NO. 13), —C(O)O— (Linker ID NO. 18), —OC(O)— (Linker ID NO. 19), —C(O)NR^{a}— (Linker ID NO. 22), —NR^{a}C(O)— (Linker ID NO. 23), —OC(O)O— (Linker ID NO. 26), —X_{AA}— (Linker ID NO. 35), —OC(O)NR^{a}— (Linker ID NO. 29), —NR^{a}C(O)O— (Linker ID NO.

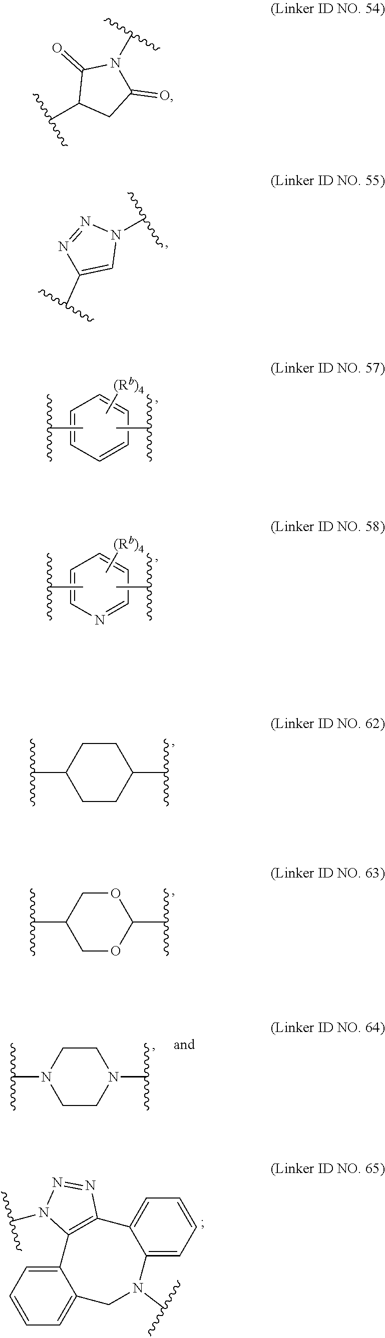

L_{P} is selected from a bond, —NR^{a′}—, and —O—;
each R₁ is independently selected from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or both R₁ groups are taken together to form optionally substituted cycloalkyl;
each R₂ is independently selected from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or both R₂ groups are taken together to form optionally substituted cycloalkyl;
each R^{a} is independently selected at each occurrence from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted alkyl;
each R^{b} is independently selected at each occurrence from hydrogen, halide, —OH, —SO₃H, —OPO₃H₂, —PO₃H₂, —CO₂R, —NR^{a}₂, optionally substituted C₁-C₈ alkyl, optionally substituted C₁-C₅ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl; and
—X_{AA}— is an amino acid sequence comprising 2 to 4 amino acid moieties.

In some embodiments, the linker L is of Formula (L-12):

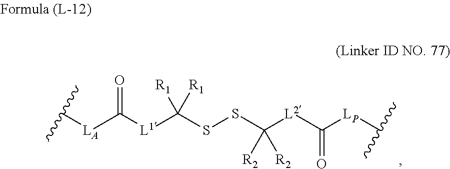

wherein in Formula (L-12):
   L_{A} is selected from a bond and —NH—;
   L^{1′} comprises one or more groups selected from —[C(R^{b})₂]₁₋₁₀— (Linker ID NO. 342), —[CH₂CH₂O]₁₋₁₀— (Linker ID NO. 343), —NR^{a}— (Linker ID NO. 1), —O— (Linker ID NO. 10), —C(O)— (Linker ID NO. 13), —C(O)O— (Linker ID NO. 18), —OC(O)— (Linker ID NO. 19), —C(O)NR^{a}— (Linker ID NO. 22), —NR^{a}C(O)— (Linker ID NO. 23), —OC(O)O— (Linker ID NO. 26), —X_{AA}— (Linker ID NO. 35), —OC(O)NR^{a}— (Linker ID NO. 29),

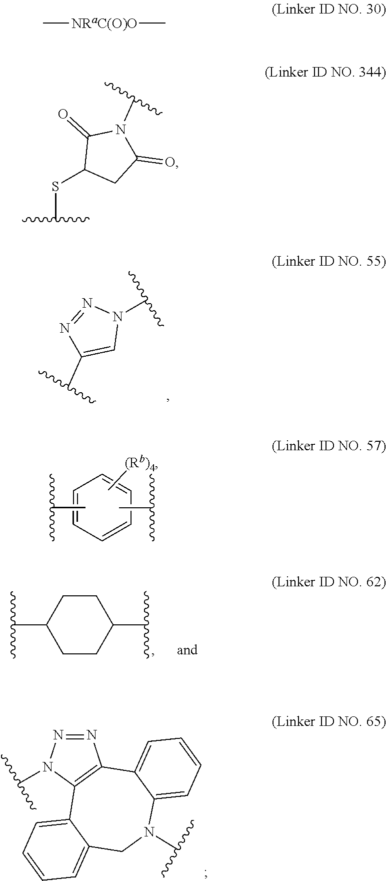

L^{2′} comprises one or more groups selected from —[C(R)₂]₁₋₁₀— (Linker ID NO. 342), —[CH₂CH₂O]₁₋₁₀— (Linker ID NO. 343), —NR^{a}— (Linker ID NO. 1), —O— (Linker ID NO. 10), —C(O)— (Linker ID NO. 13), —C(O)O— (Linker ID NO. 18), —OC(O)— (Linker ID NO. 19), —C(O)NR^{a}— (Linker ID NO. 22), —NR^{a}C(O)— (Linker ID NO. 23), —OC(O)O— (Linker ID NO. 26), —X_{AA}— (Linker ID NO. 35), —OC(O)NR^{a}— (Linker ID NO. 29),

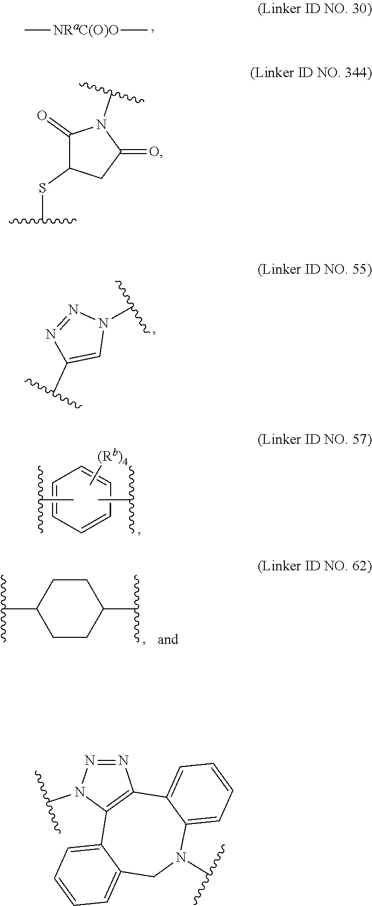

L_{P} is selected from a bond and —NR^{a′}- (Linker ID NO. 1);
each R₁ is independently selected from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or both R₁ groups are taken together to form optionally substituted C₃-C₆ cycloalkyl;
each R₂ is independently selected from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or both R₂ groups are taken together to form optionally substituted C₃-C₆ cycloalkyl;
each R^{a} is independently selected at each occurrence from hydrogen, optionally substituted C₁-C₆ alkyl, optionally substituted C₁-C₆ fluoroalkyl, and optionally substituted C₃-C₆ cycloalkyl;
each R^{a′} is independently selected at each occurrence from hydrogen and optionally substituted C₁-C₆ alkyl;
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₆ alkyl, optionally substituted C₁-C₆ fluoroalkyl, and optionally substituted C₃-C₆ cycloalkyl; or two independent R^{b} groups attached to the same carbon atom are taken together to form optionally substituted C₃-C₆ cycloalkyl; and
—X_{AA}— is an amino acid sequence comprising 2 or 3 amino acid moieties.

In some embodiments, at least one R₁ or R₂ is other than hydrogen. In some embodiments, at least one R₁ is an optionally substituted C₁-C₈ alkyl. In some embodiments, each R₁ is independently an optionally substituted C₁-C₈ alkyl. In some embodiments, at least one R₂ is an optionally substituted C₁-C₈ alkyl. In some embodiments, each R₂ is independently an optionally substituted C₁-C₈ alkyl.

In some embodiments, the linker L is selected from:

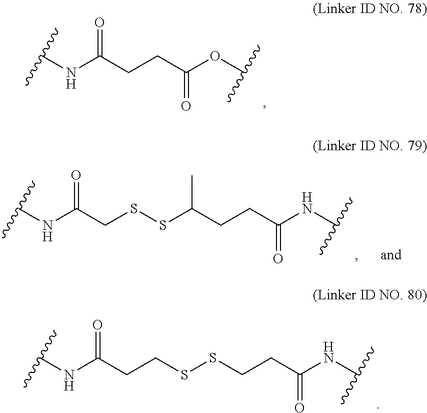 [SMILES: *NC(=O)CCC(=O)O* ; *NC(=O)CCC(C)SSCC(=O)N* ; *NC(=O)CCSSCCC(=O)N*]

In some embodiments, the linker L is selected from:

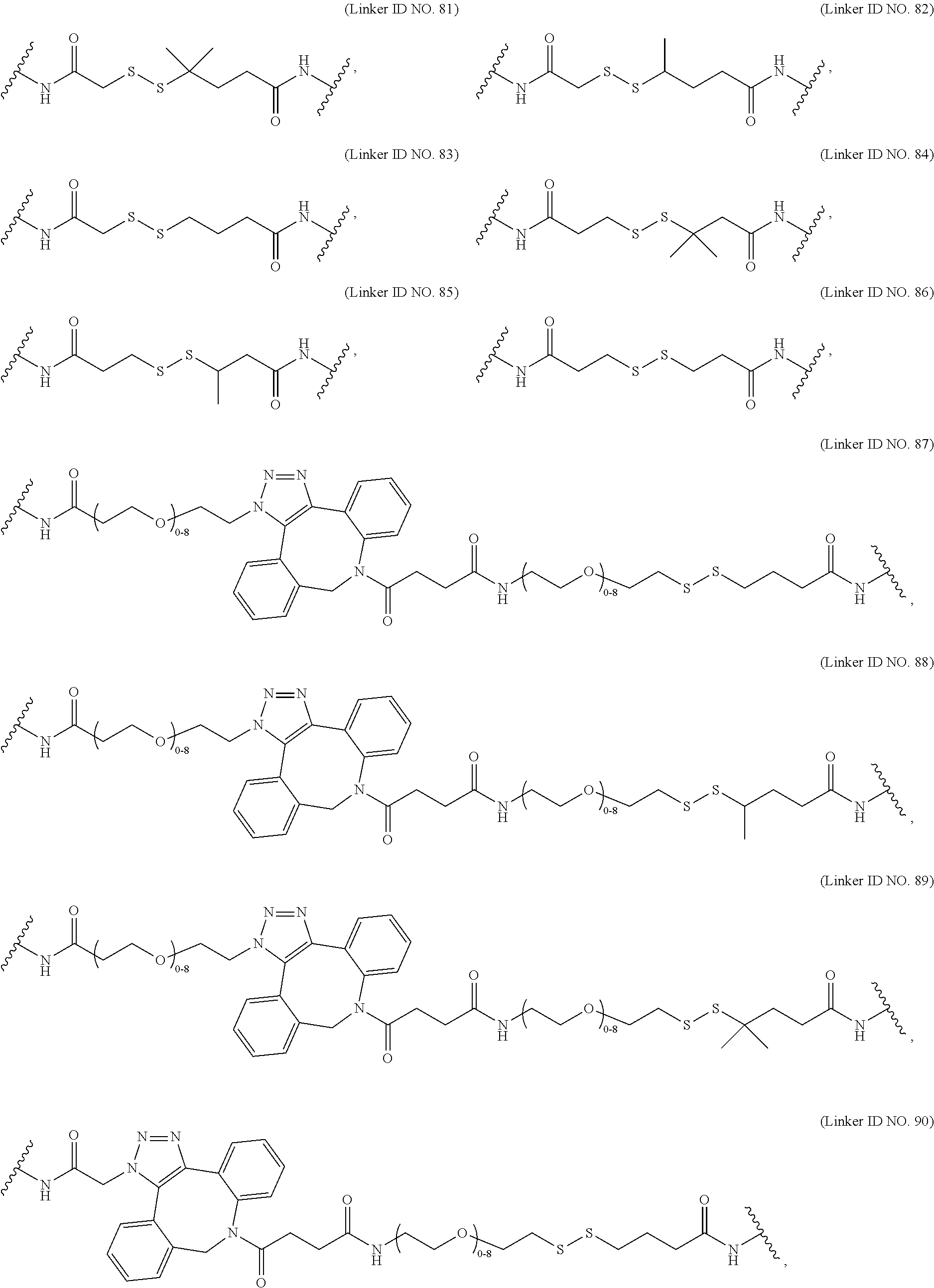

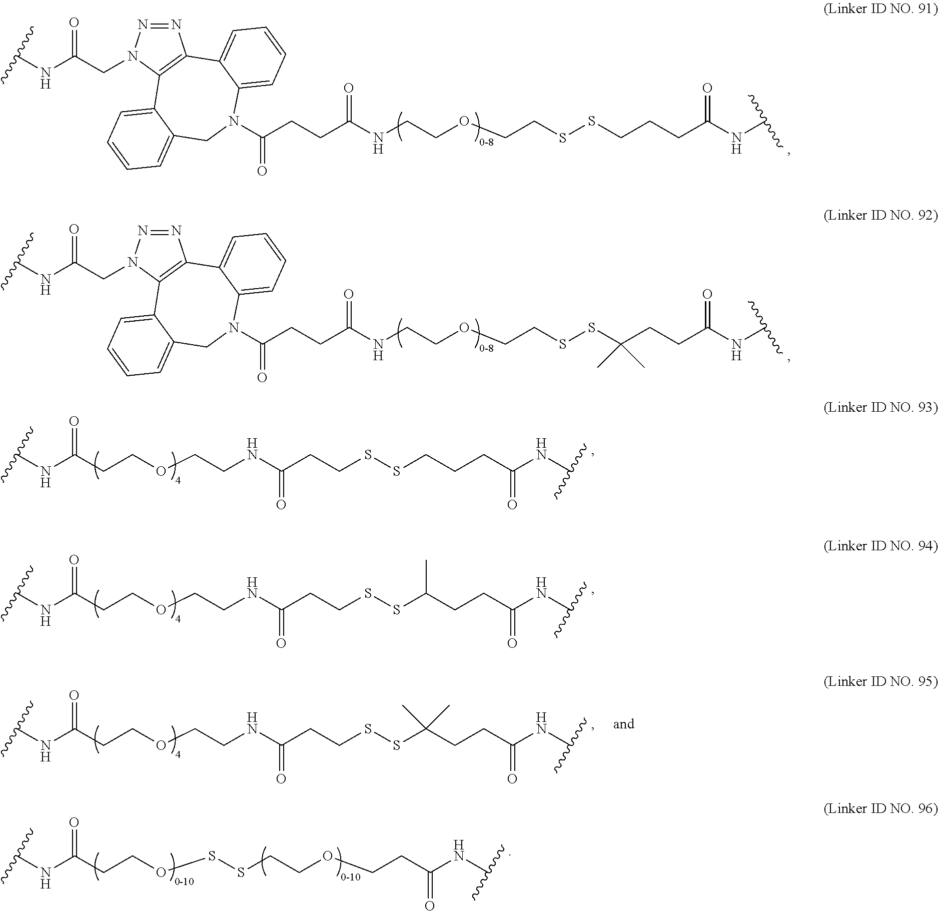

In some embodiments, the linker is a cleavable linker.

In some embodiments, the linker L is of Formula (L-20):

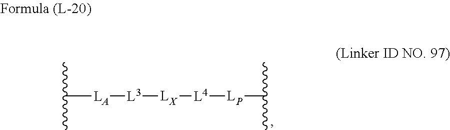

wherein in Formula (L-20):
   L^{A} is selected from a bond, —NR^{a′}- (Linker ID NO. 1), and —S— (Linker ID NO. 4); L³ is a bond or comprises one or more groups selected from —[C(R^{b})₂]₁₋₈-, —N^{a}—, —C(O)—, —C(S)—, —C(NR^{a})—, —C(NH₂Cl)—, —C≡C—, —CR^{a}—CR^{a}—, optionally substituted 6- to 14-membered arylene, optionally substituted C₃-C₂₀ cycloalkylene, optionally substituted 5- to 18-membered heteroarylene, and optionally substituted 3- to 20-membered heterocycloalkylene;
   L_{X} comprises one or more groups selected from optionally substituted C₁-C₁₈ alkylene, —C≡C—, —CR^{a}—CR^{a}—, optionally substituted 6- to 14-membered arylene, optionally substituted C₃-C₂₀ cycloalkylene, —[CH₂O]₁₋₁₈—, —[CH₂CH₂O]₁₋₁₈₋, —[CH₂CH₂CH₂O]₁₋₁₈—, optionally substituted 5- to 18-membered heteroarylene, optionally substituted 3- to 20-membered heterocycloalkylene, —NR^{a}—, —S—, —O—, —CR^{b}₂—, —C(O)—, —C(S)—, —C(NR^{a})—, —C(NH₂Cl)—, —C(O)O—, —OC(O)—, —C(O)S—, —SC(O)—, —C(O)NR^{a}—, and —NR^{a}C(O)—;
   L⁴ is a bond or comprises one or more groups selected from —[C(R^{b})₂]₁-s-, —NR^{a}—, —C(O)—, —C(S)—, —C(NR^{a})—, —C(NH₂Cl)—, —C≡C—, —CR^{a}—CR^{a}—, optionally substituted 6- to 14-membered arylene, optionally substituted C₃-C₂₀ cycloalkylene, optionally substituted 5- to 18-membered heteroarylene, and optionally substituted 3- to 20-membered heterocycloalkylene;
   L_{P} is selected from a bond, —NR^{a′}—, —S—, and —O—;
   each R^{a} is independently selected at each occurrence from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
   each R^{a′} is independently selected at each occurrence from hydrogen, optionally substituted alkyl, and optionally substituted heteroalkyl; and
   each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or two independent R^{b} groups are taken together to form optionally substituted cycloalkyl, optionally substituted aryl, optionally substituted heterocycloalkyl, or optionally substituted heteroaryl.

In some embodiments, the linker L is of Formula (L-21):

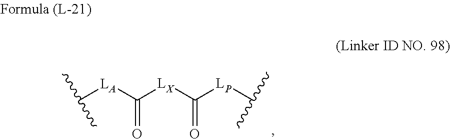

wherein in Formula (L-21):
   L_{A} is selected from a bond and —NH—;
   L_{X} comprises one or more groups selected from optionally substituted —[C(R^{b})₂]₁₋₁₆—, —C≡C—, —CR^{a}—CR^{a}—, —[CH₂CH₂CH₂O]₁₋₁₆—, —NR^{a}—, —O—, —C(O)—, —C(O)O—, —OC(O)—, —C(O)S—,

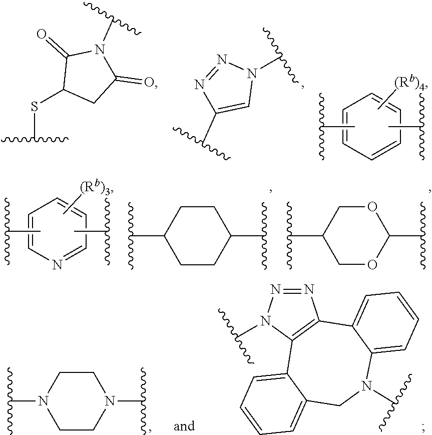

L_{P} is selected from a bond and —NR^{a′};
each R^{a} is independently selected at each occurrence from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted C₁-C₆ alkyl; and
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl.

In some embodiments, the linker L is of Formula (L-22a) or Formula (L-22b):

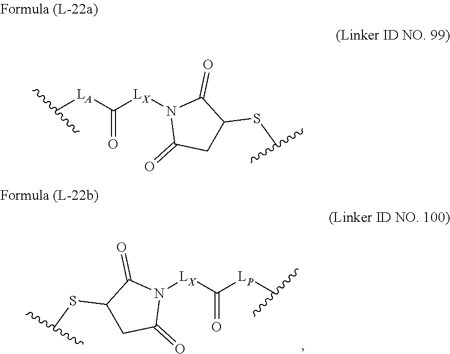

wherein in Formulas (L-22a) and (L-22b):
   L_{A} is selected from a bond and —NH—;
   L_{X} comprises one or more groups selected from optionally substituted —[C(R^{b})₂]₁₋₁₀—, —C≡C—, —CR^{a}—CR^{a}—, —[CH₂CH₂CH₂O]₁₋₁₀—, —NR^{a}—, —C(O)—, —C(O)O—, —OC(O)—, —C(O)S—, —SC(O)—, —C(O)NR^{a}—, —NR^{a}C(O)—,

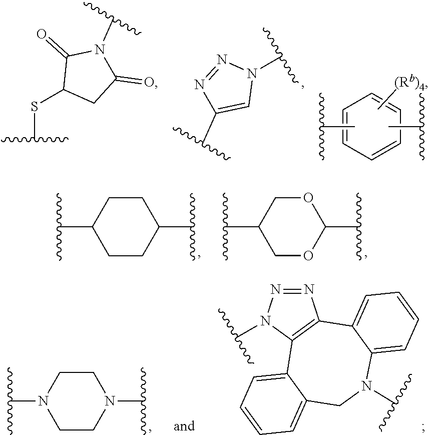

L_{P} is selected from a bond and —NR^{a′};
each R^{a} is independently selected at each occurrence from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted C₁-C₆ alkyl; and
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₅ alkyl, optionally substituted C₁-C₅ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl.

In some embodiments, the linker L is selected from:

| | Linker |
|---|---|
| | ID |
| Linker | NO. |
| 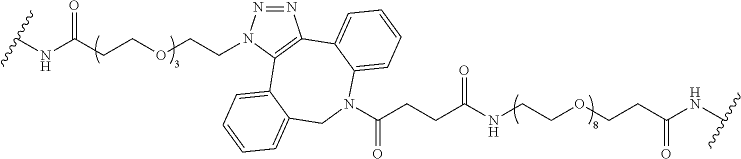 | 101 |
| 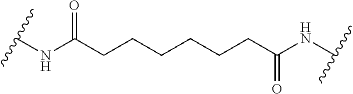 [SMILES: *NC(=O)CCCCCCC(=O)N*] | 102 |
| 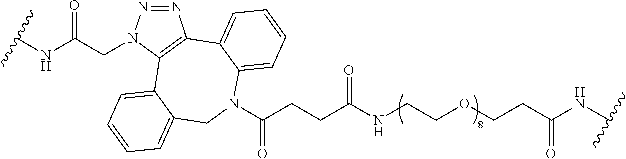 | 103 |
| 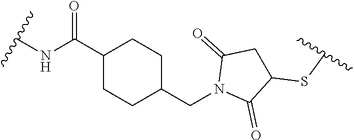 [SMILES: *NC(=O)C1CCC(CN2C(=O)CC(S*)C2=O)CC1] | 104 |
| 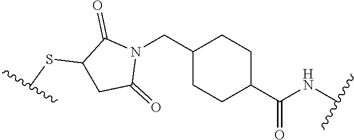 [SMILES: *NC(=O)C1CCC(CN2C(=O)CC(S*)C2=O)CC1] | 105 |
| 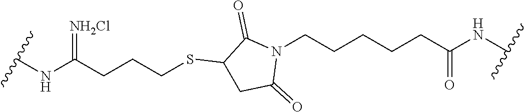 | 106 |
| 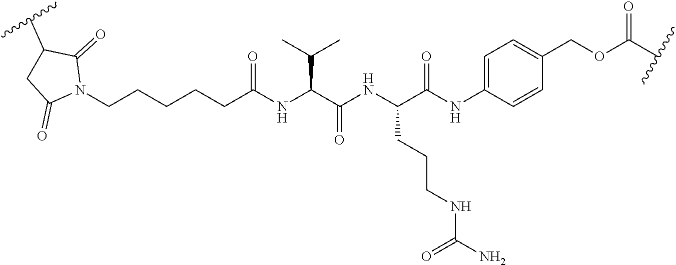 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1] | 107 |
| 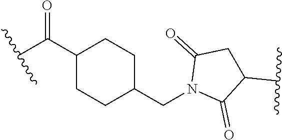 [SMILES: *C(=O)C1CCC(CN2C(=O)CC(*)C2=O)CC1] | 108 |
| 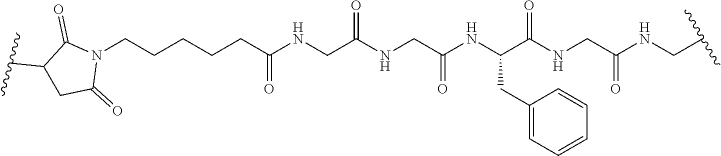 [SMILES: *CNC(=O)CNC(=O)[C@H](Cc1ccccc1)NC(=O)CNC(=O)CNC(=O)CCCCCN1C(=O)CC(*)C1=O] | 109 |
| 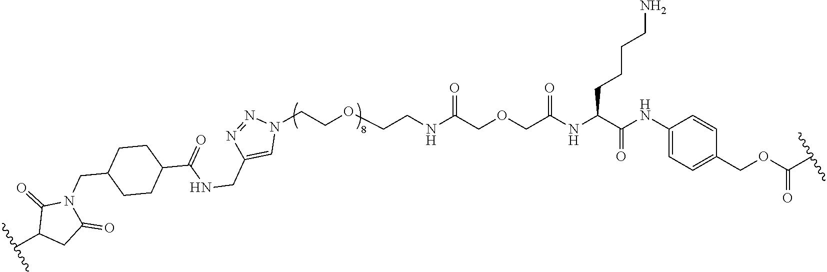 | 110 |
| 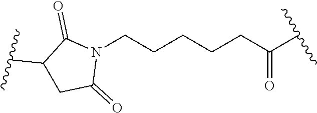 [SMILES: *C(=O)CCCCCN1C(=O)CC(*)C1=O] | 111 |
| 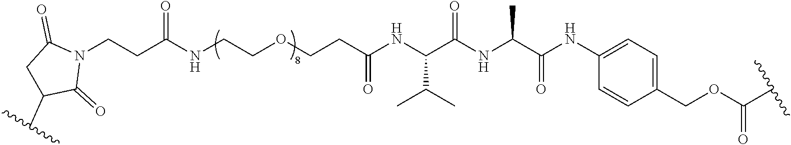 | 112 |
| 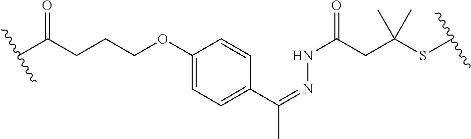 [SMILES: *SC(C)(C)CC(=O)N/N=C(/C)c1ccc(OCCCC(*)=O)cc1] | 113 |
| 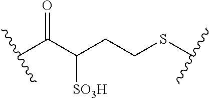 [SMILES: *SCCC(C(*)=O)S(=O)(=O)O] | 114 |
| 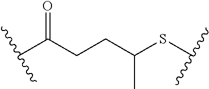 [SMILES: *SC(C)CCC(*)=O] | 115 |
| 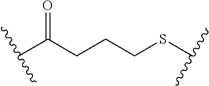 [SMILES: *SCCCC(*)=O] | 116 |
| 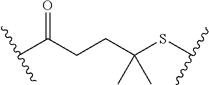 [SMILES: *SC(C)(C)CCC(*)=O] | 117 |
| 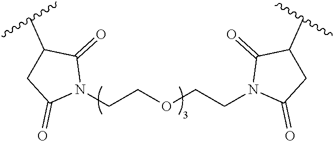 | 118 |
| 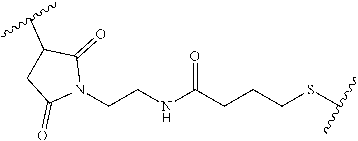 [SMILES: *SCCCC(=O)NCCN1C(=O)CC(*)C1=O] | 119 |
| 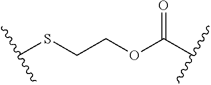 [SMILES: *SCCOC(*)=O] | 120 |
| 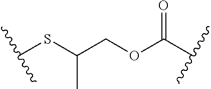 [SMILES: *SC(C)COC(*)=O] | 121 |
| 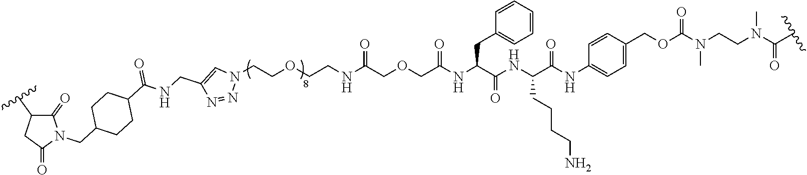 | 122 |
| 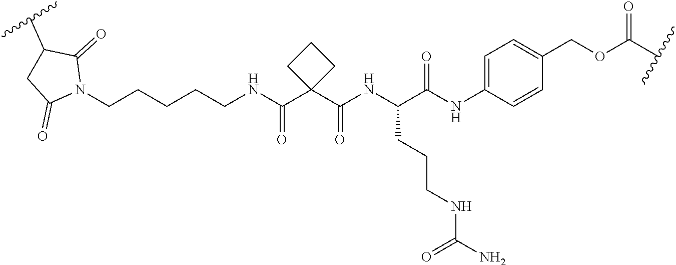 [SMILES: *C(=O)OCc1ccc(NC(=O)[C@H](CCCNC(N)=O)NC(=O)C2(C(=O)NCCCCCN3C(=O)CC(*)C3=O)CCC2)cc1] | 123 |
| 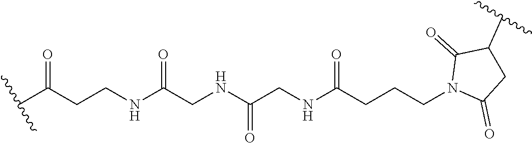 [SMILES: *C(=O)CCNC(=O)CNC(=O)CNC(=O)CCCN1C(=O)CC(*)C1=O] | 124 |
| 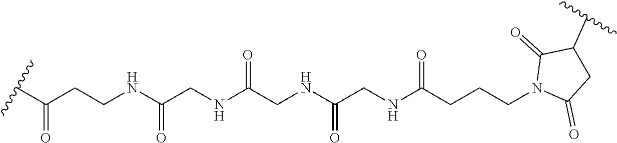 [SMILES: *C(=O)CCNC(=O)CNC(=O)CNC(=O)CNC(=O)CCCN1C(=O)CC(*)C1=O] | 125 |
| 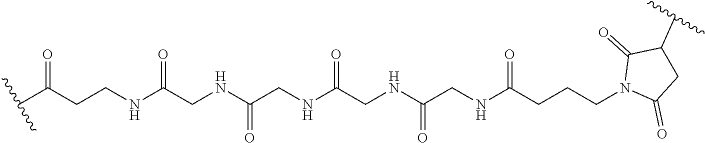 [SMILES: *C(=O)CCNC(=O)CNC(=O)CNC(=O)CNC(=O)CNC(=O)CCCN1C(=O)CC(*)C1=O] | 126 |
| 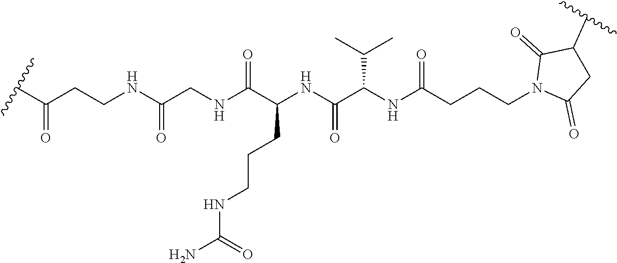 [SMILES: *C(=O)CCNC(=O)CNC(=O)[C@H](CCCNC(N)=O)NC(=O)[C@@H](NC(=O)CCCN1C(=O)CC(*)C1=O)C(C)C] | 127 |
| 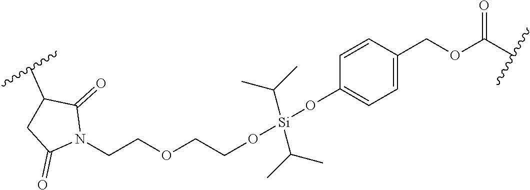 [SMILES: *C(=O)OCc1ccc(O[Si](OCCOCCN2C(=O)CC(*)C2=O)(C(C)C)C(C)C)cc1] | 128 |
| 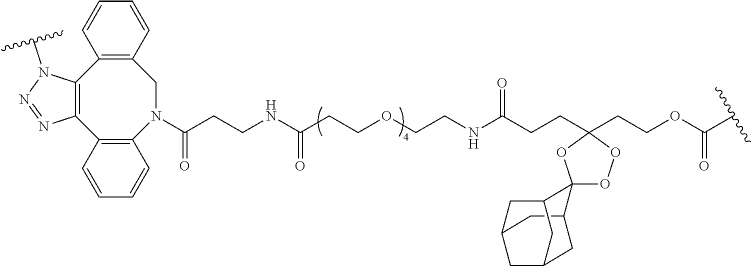 | 129 |
| 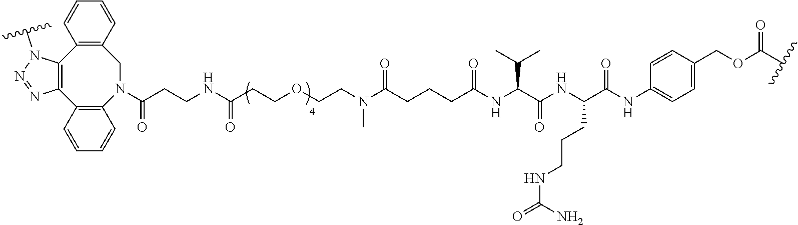 | 130 |
| 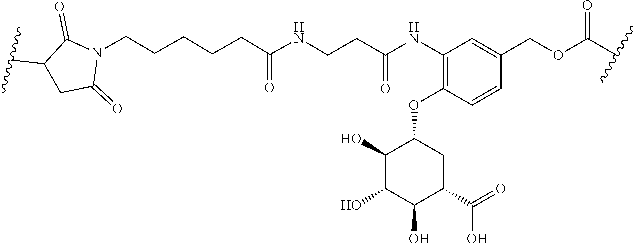 [SMILES: *C(=O)OCc1ccc(O[C@@H]2C[C@H](C(=O)O)[C@@H](O)[C@H](O)[C@H]2O)c(NC(=O)CCNC(=O)CCCCCN2C(=O)CC(*)C2=O)c1] | 131 |
| 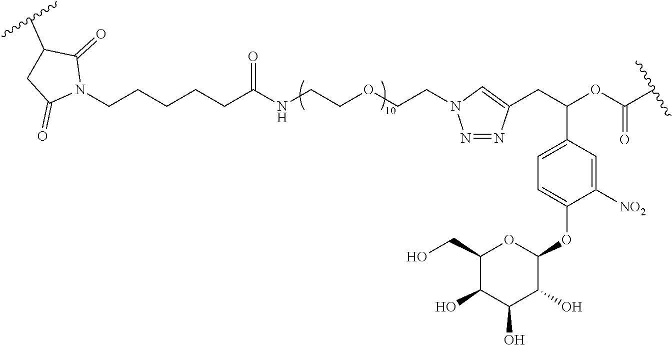 | 132 |
| 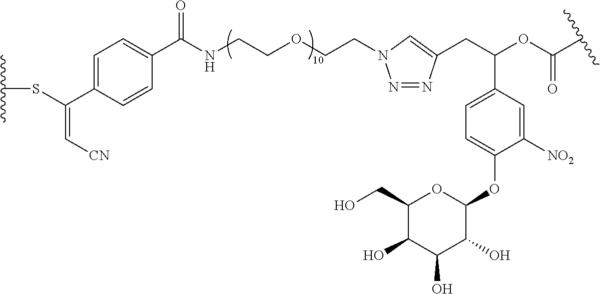 | 133 |
| 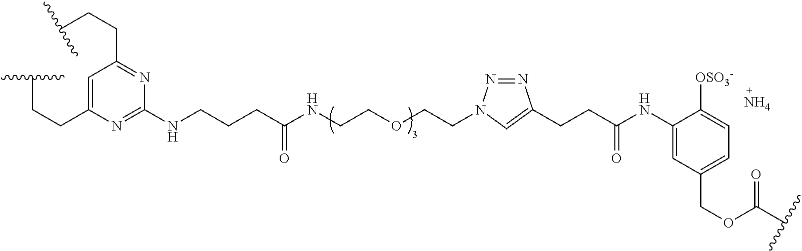 | 134 |
| 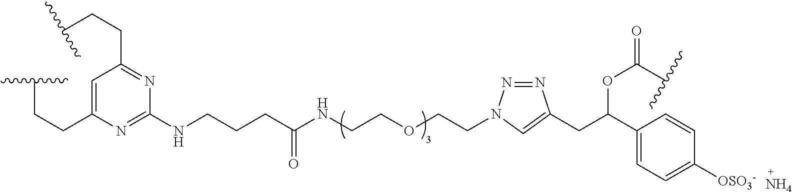 | 135 |
| 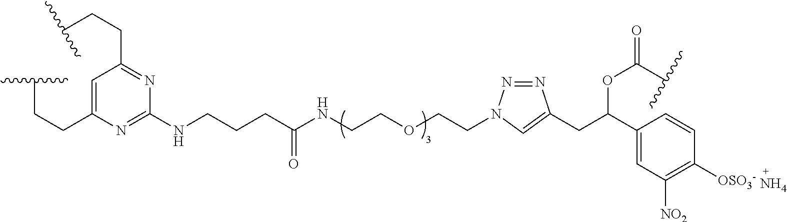 | 136 |
| 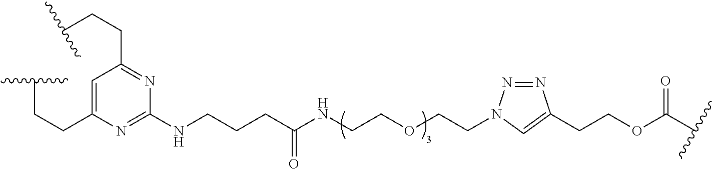 | 137 |
| 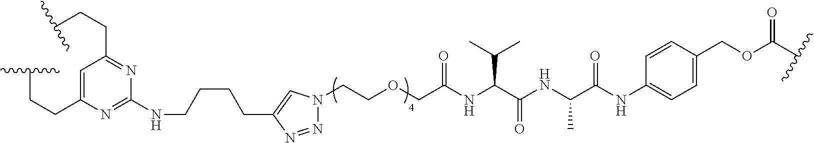 | 138 |
| 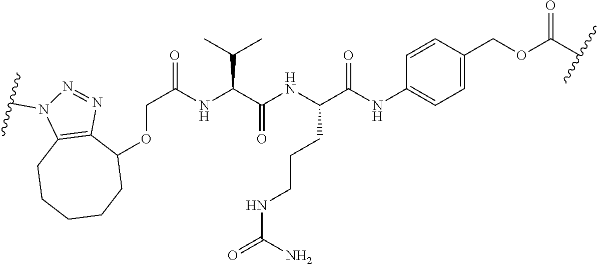 [SMILES: *C(=O)OCc1ccc(NC(=O)[C@H](CCCNC(N)=O)NC(=O)[C@@H](NC(=O)COC2CCCCCc3c2nnn3*)C(C)C)cc1] | 139 |
| 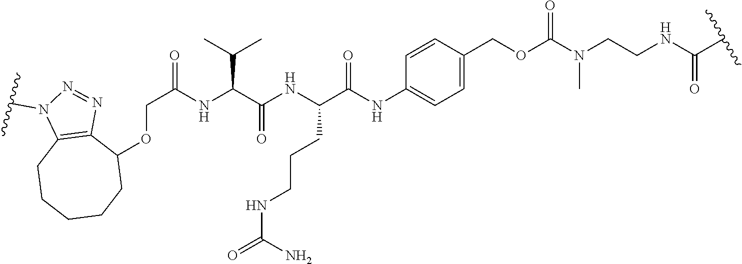 [SMILES: *C(=O)NCCN(C)C(=O)OCc1ccc(NC(=O)[C@H](CCCNC(N)=O)NC(=O)[C@@H](NC(=O)COC2CCCCCc3c2nnn3*)C(C)C)cc1] | 140 |
| 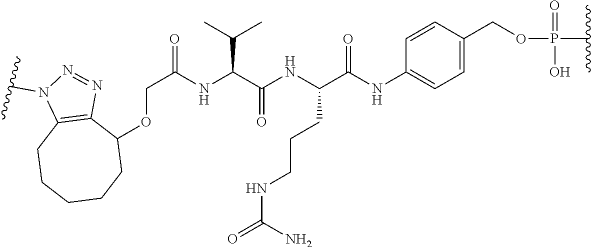 [SMILES: *n1nnc2c1CCCCCC2OCC(=O)N[C@H](C(=O)N[C@@H](CCCNC(N)=O)C(=O)Nc1ccc(COP(*)(=O)O)cc1)C(C)C] | 141 |
| 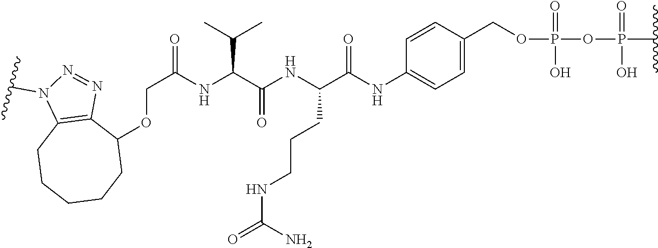 [SMILES: *n1nnc2c1CCCCCC2OCC(=O)N[C@H](C(=O)N[C@@H](CCCNC(N)=O)C(=O)Nc1ccc(COP(=O)(O)OP(*)(=O)O)cc1)C(C)C] | 142 |
| 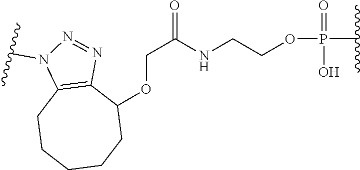 [SMILES: *n1nnc2c1CCCCCC2OCC(=O)NCCOP(*)(=O)O] | 143 |
| 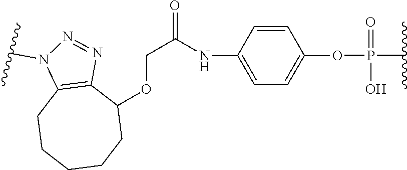 [SMILES: *n1nnc2c1CCCCCC2OCC(=O)Nc1ccc(OP(*)(=O)O)cc1] | 144 |
| 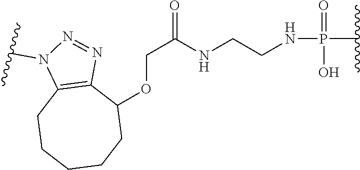 [SMILES: *n1nnc2c1CCCCCC2OCC(=O)NCCNP(*)(=O)O] | 145 |
| 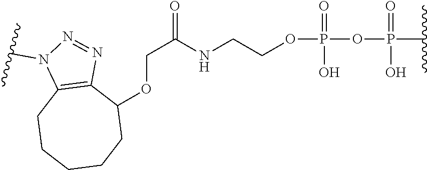 [SMILES: *n1nnc2c1CCCCCC2OCC(=O)NCCOP(=O)(O)OP(*)(=O)O] | 146 |
| 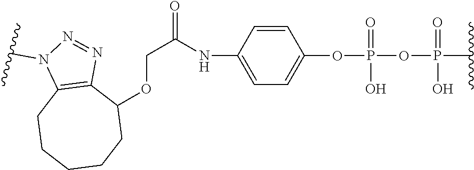 [SMILES: *n1nnc2c1CCCCCC2OCC(=O)Nc1ccc(OP(=O)(O)OP(*)(=O)O)cc1] | 147 |
| 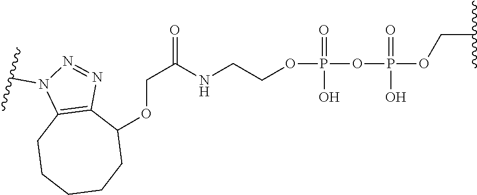 [SMILES: *COP(=O)(O)OP(=O)(O)OCCNC(=O)COC1CCCCCc2c1nnn2*] | 148 |
| 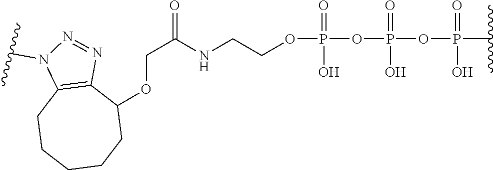 [SMILES: *n1nnc2c1CCCCCC2OCC(=O)NCCOP(=O)(O)OP(=O)(O)OP(*)(=O)O] | 149 |
| 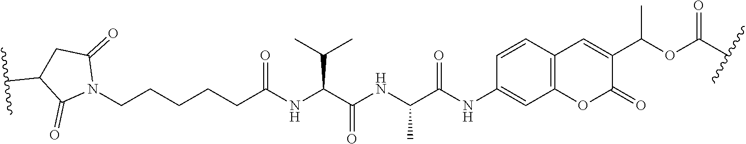 [SMILES: *C(=O)OC(C)c1cc2ccc(NC(=O)[C@H](C)NC(=O)[C@@H](NC(=O)CCCCCN3C(=O)CC(*)C3=O)C(C)C)cc2oc1=O] | 150 |
| 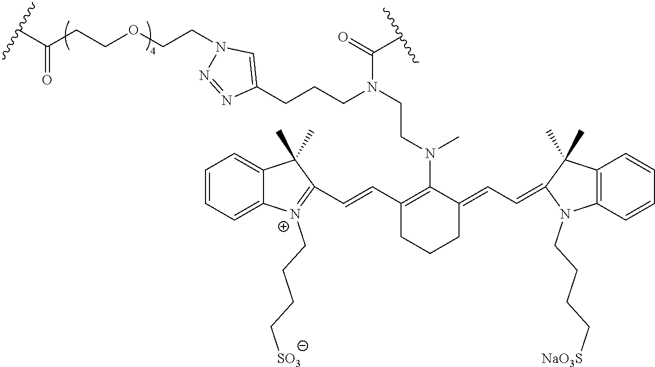 | 151 |
| 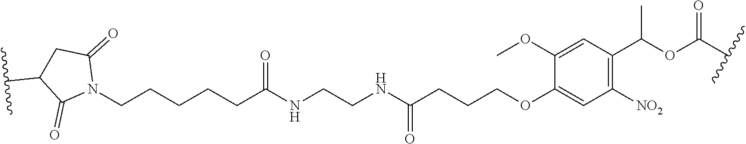 [SMILES: *C(=O)OC(C)c1cc(OC)c(OCCCC(=O)NCCNC(=O)CCCCCN2C(=O)CC(*)C2=O)cc1[N+](=O)[O-]] | 152 |
| 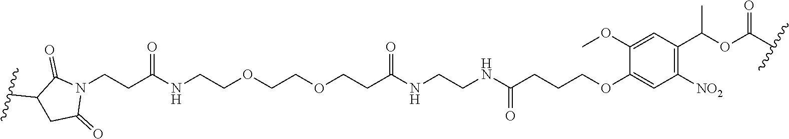 [SMILES: *C(=O)OC(C)c1cc(OC)c(OCCCC(=O)NCCNC(=O)CCOCCOCCNC(=O)CCN2C(=O)CC(*)C2=O)cc1[N+](=O)[O-]] | 153 |
| 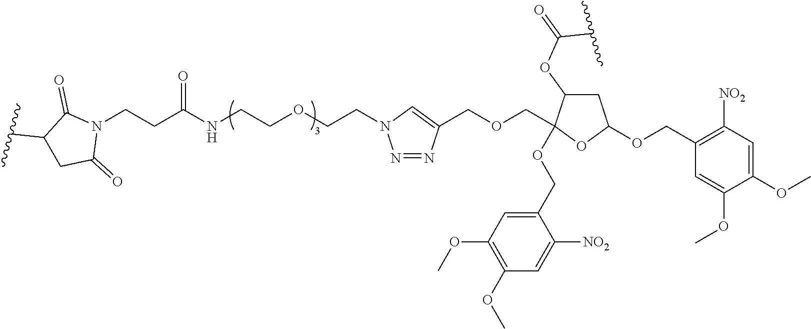 | 154 |
| 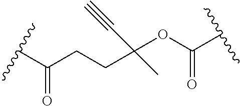 [SMILES: *C(=O)CCC(C)(C#C)OC(*)=O] | 155 |
| 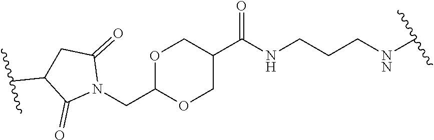 | 156 |
| 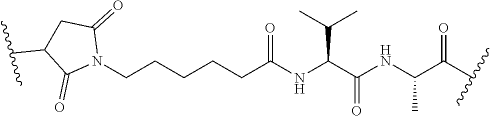 [SMILES: *C(=O)[C@H](C)NC(=O)[C@@H](NC(=O)CCCCCN1C(=O)CC(*)C1=O)C(C)C] | 157 |
| 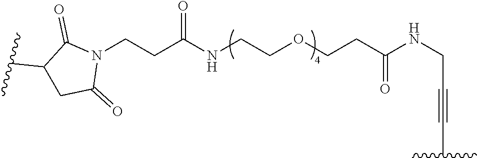 | 158 |
| 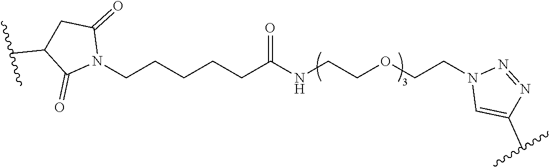 | 159 |
| 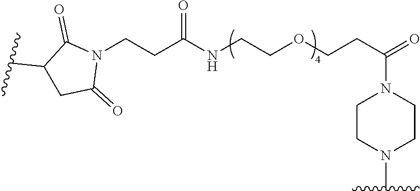 | 160 |
| 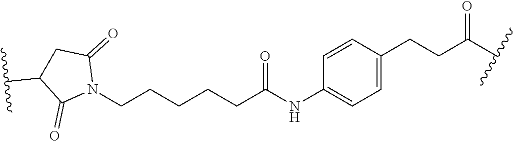 [SMILES: *C(=O)CCc1ccc(NC(=O)CCCCCN2C(=O)CC(*)C2=O)cc1] | 161 |
| 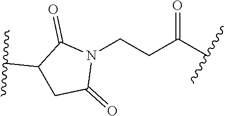 [SMILES: *C(=O)CCN1C(=O)CC(*)C1=O] | 162 |
| 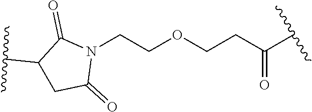 [SMILES: *C(=O)CCOCCN1C(=O)CC(*)C1=O] | 163 |
| 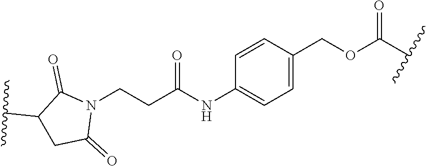 [SMILES: *C(=O)OCc1ccc(NC(=O)CCN2C(=O)CC(*)C2=O)cc1] | 164 |
| 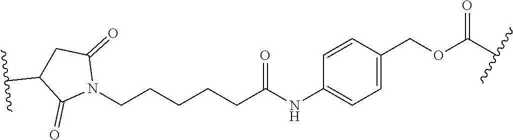 [SMILES: *C(=O)OCc1ccc(NC(=O)CCCCCN2C(=O)CC(*)C2=O)cc1] | 165 |
| 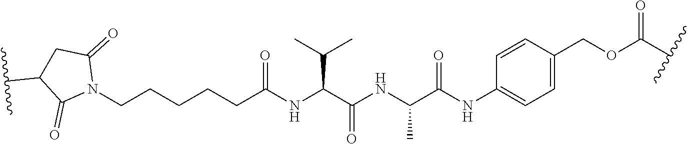 [SMILES: *C(=O)OCc1ccc(NC(=O)[C@H](C)NC(=O)[C@@H](NC(=O)CCCCCN2C(=O)CC(*)C2=O)C(C)C)cc1] | 166 |
| 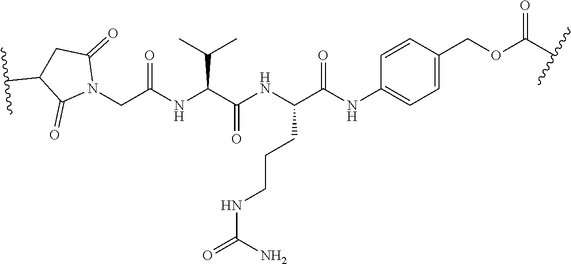 [SMILES: *C(=O)OCc1ccc(NC(=O)[C@H](CCCNC(N)=O)NC(=O)[C@@H](NC(=O)CN2C(=O)CC(*)C2=O)C(C)C)cc1] | 167 |
| 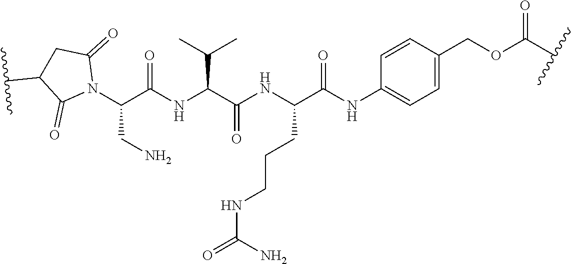 [SMILES: *C(=O)OCc1ccc(NC(=O)[C@H](CCCNC(N)=O)NC(=O)[C@@H](NC(=O)[C@H](CN)N2C(=O)CC(*)C2=O)C(C)C)cc1] | 168 |
| 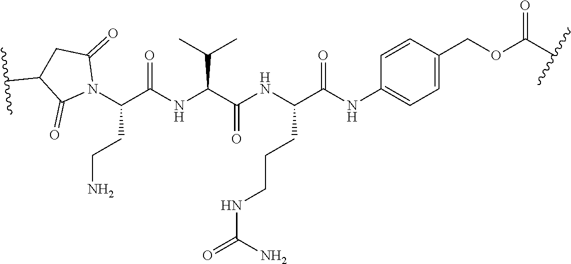 [SMILES: *C(=O)OCc1ccc(NC(=O)[C@H](CCCNC(N)=O)NC(=O)[C@@H](NC(=O)[C@H](CCN)N2C(=O)CC(*)C2=O)C(C)C)cc1] | 169 |
| 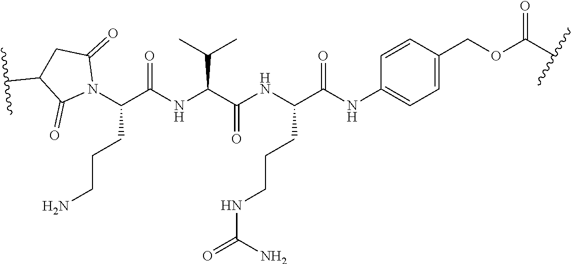 [SMILES: *C(=O)OCc1ccc(NC(=O)[C@H](CCCNC(N)=O)NC(=O)[C@@H](NC(=O)[C@H](CCCN)N2C(=O)CC(*)C2=O)C(C)C)cc1] | 170 |
| 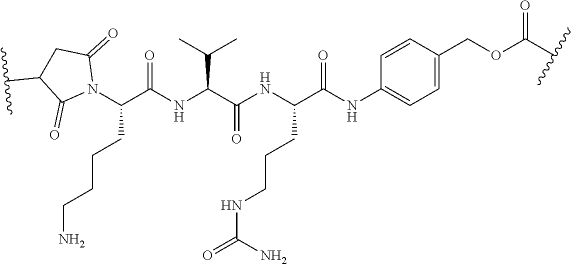 [SMILES: *C(=O)OCc1ccc(NC(=O)[C@H](CCCNC(N)=O)NC(=O)[C@@H](NC(=O)[C@H](CCCCN)N2C(=O)CC(*)C2=O)C(C)C)cc1] | 171 |
| 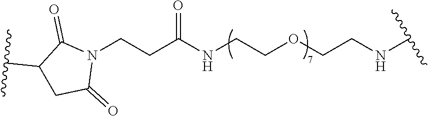 | 172 |
| 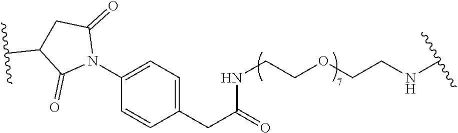 | 173 |
| 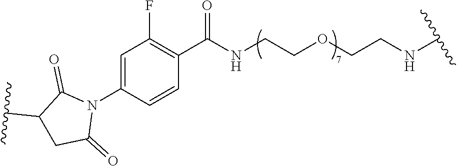 | 174 |
| 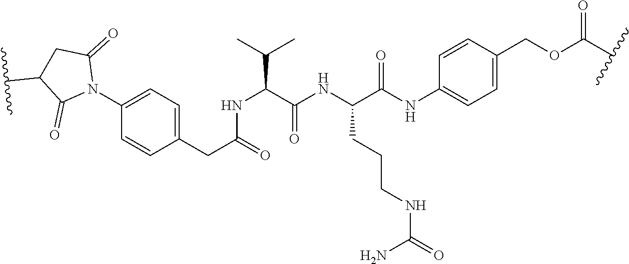 [SMILES: *C(=O)OCc1ccc(NC(=O)[C@H](CCCNC(N)=O)NC(=O)[C@@H](NC(=O)Cc2ccc(N3C(=O)CC(*)C3=O)cc2)C(C)C)cc1] | 175 |
| 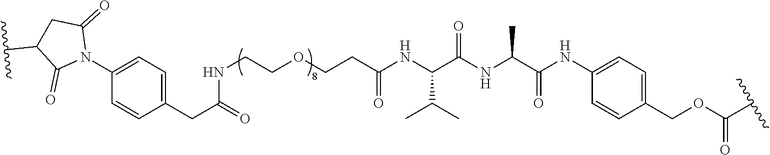 | 176 |
| 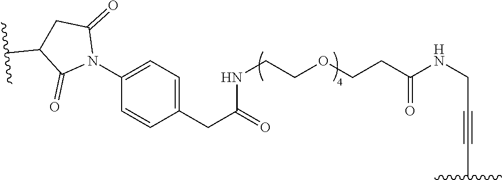 | 177 |
| 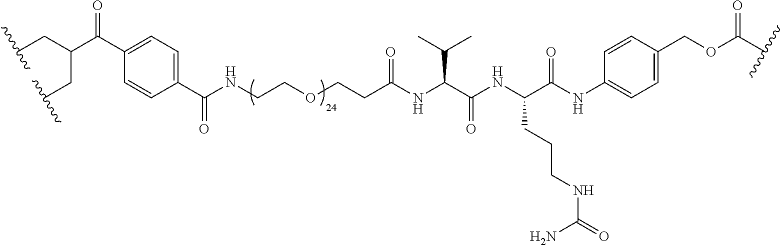 | 178 |
| 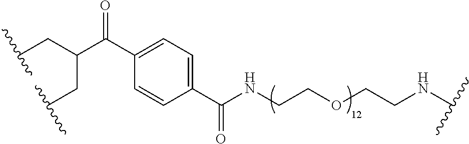 | 179 |
| 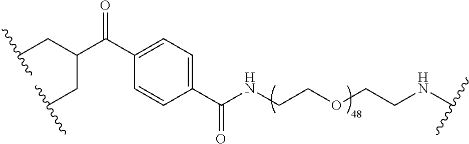 | 180 |
| 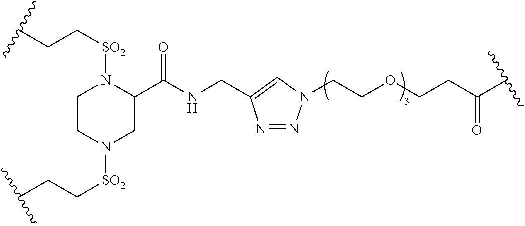 | 181 |
| 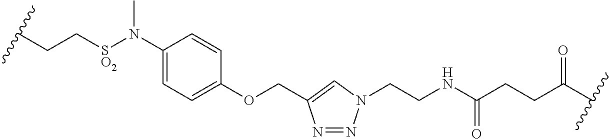 [SMILES: *CCS(=O)(=O)N(C)c1ccc(OCc2cn(CCNC(=O)CCC(*)=O)nn2)cc1] | 182 |
| 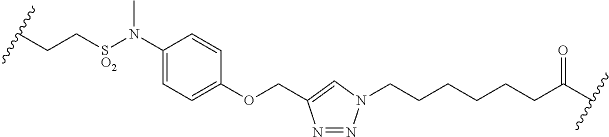 [SMILES: *CCS(=O)(=O)N(C)c1ccc(OCc2cn(CCCCCCC(*)=O)nn2)cc1] | 183 |
| 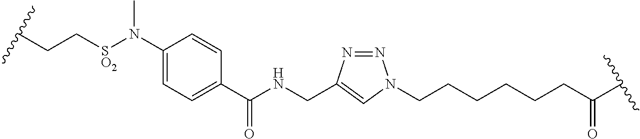 [SMILES: *CCS(=O)(=O)N(C)c1ccc(C(=O)NCc2cn(CCCCCCC(*)=O)nn2)cc1] | 184 |
| 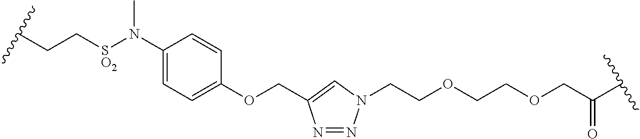 [SMILES: *CCS(=O)(=O)N(C)c1ccc(OCc2cn(CCOCCOCC(*)=O)nn2)cc1] | 185 |
| 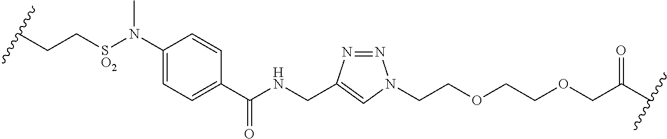 [SMILES: *CCS(=O)(=O)N(C)c1ccc(C(=O)NCc2cn(CCOCCOCC(*)=O)nn2)cc1] | 186 |
| 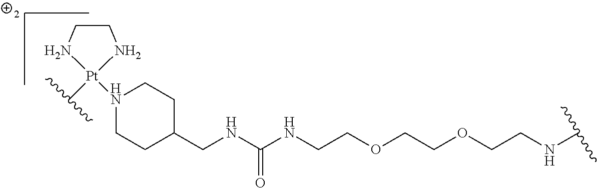 | 187 |
| 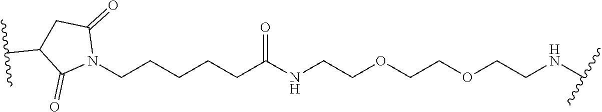 [SMILES: *NCCOCCOCCNC(=O)CCCCCN1C(=O)CC(*)C1=O] | 188 |
| 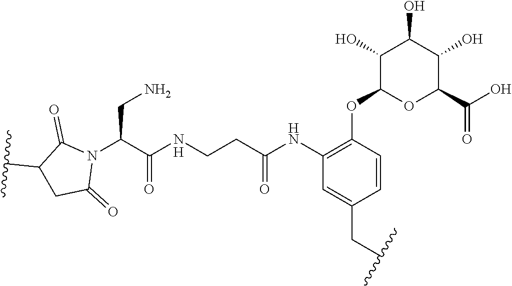 | 189 |
| 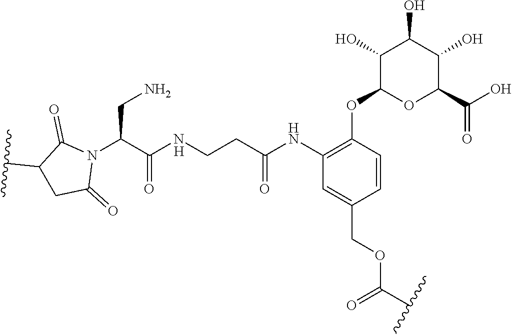 | 190 |
| 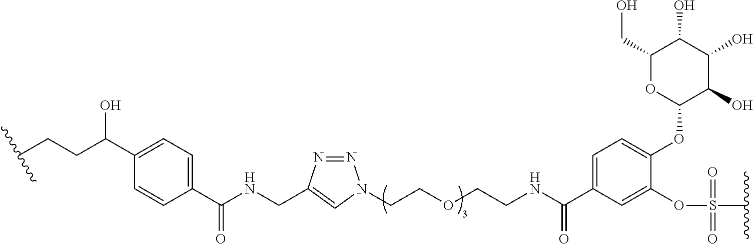 | 191 |
| 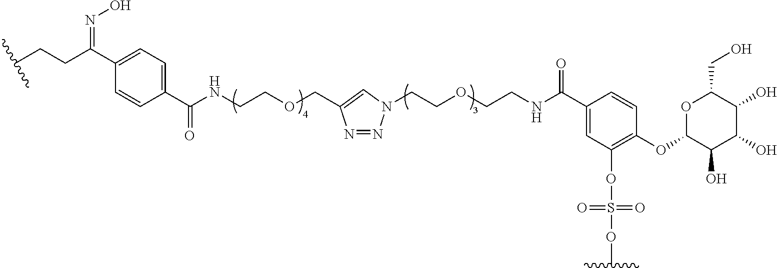 | 192 |
| 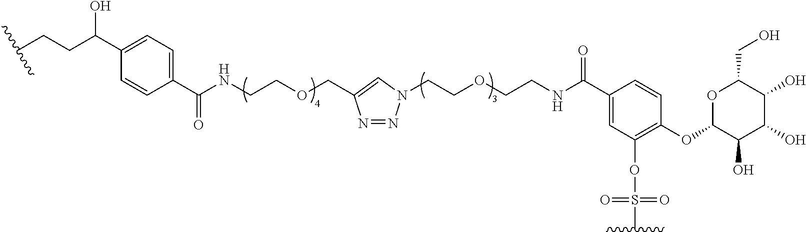 | 193 |
| 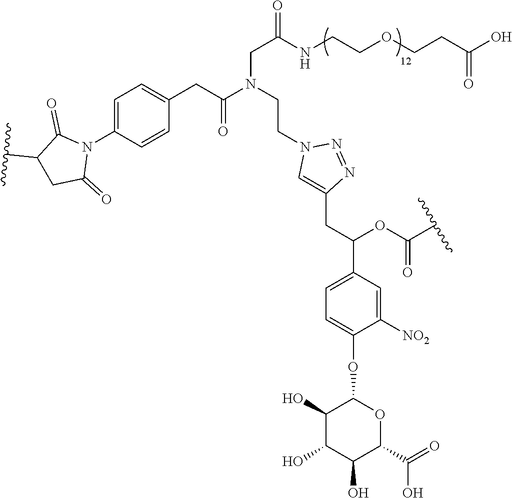 | 194 |
| 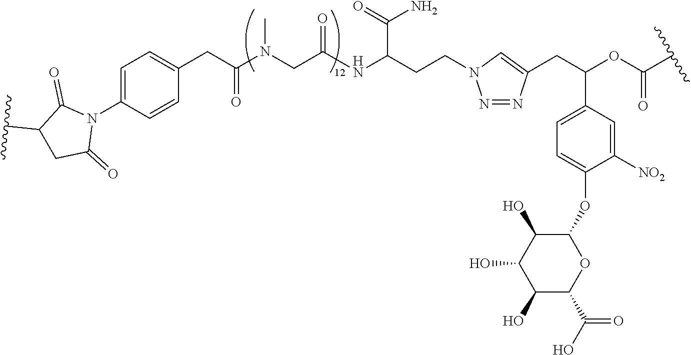 | 195 |
| 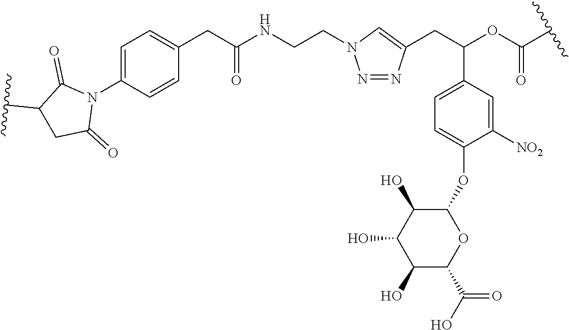 [SMILES: *C(=O)OC(Cc1cn(CCNC(=O)Cc2ccc(N3C(=O)CC(*)C3=O)cc2)nn1)c1ccc(O[C@@H]2O[C@H](C(=O)O)[C@@H](O)[C@H](O)[C@H]2O)c([N+](=O)[O-])c1] | 196 |
| 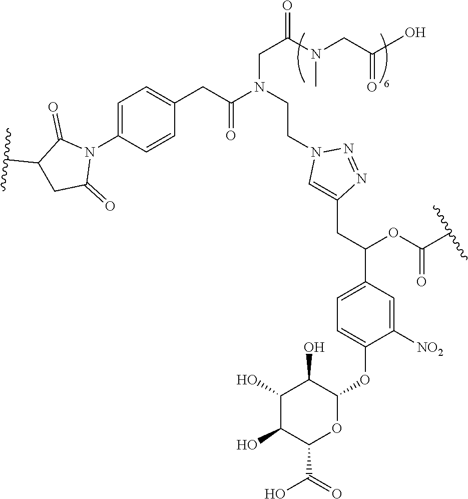 | 197 |
| 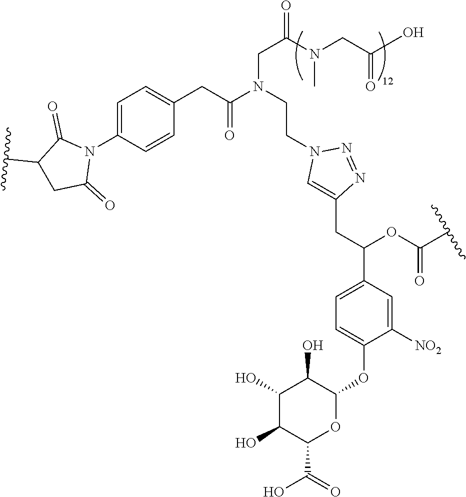 | 198 |
| 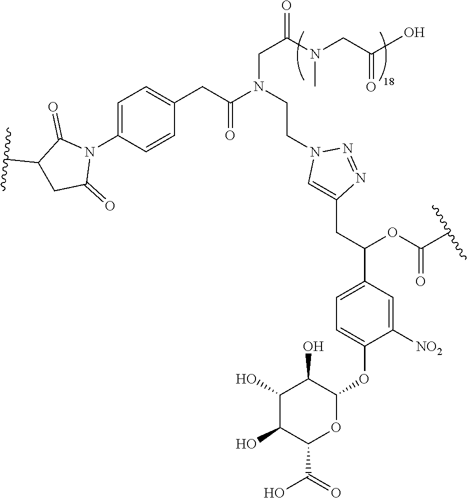 | 199 |
| 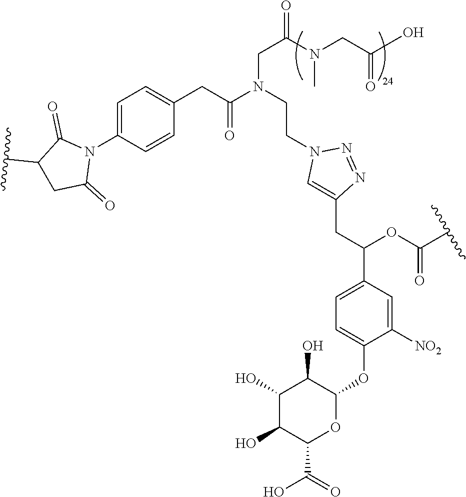 | 200 |
| 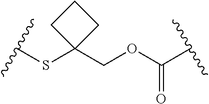 [SMILES: *SC1(COC(*)=O)CCC1] | 201 |
| 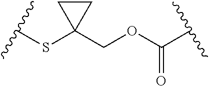 [SMILES: *SC1(COC(*)=O)CC1] | 202 |
| 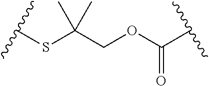 [SMILES: *SC(C)(C)COC(*)=O] | 203 |
| 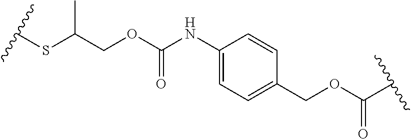 [SMILES: *SC(C)COC(=O)Nc1ccc(COC(*)=O)cc1] | 204 |
| 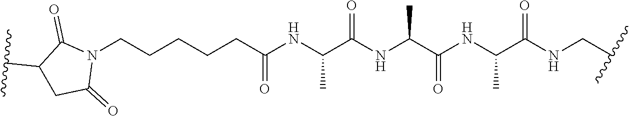 [SMILES: *CNC(=O)[C@H](C)NC(=O)[C@H](C)NC(=O)[C@H](C)NC(=O)CCCCCN1C(=O)CC(*)C1=O] | 205 |
| 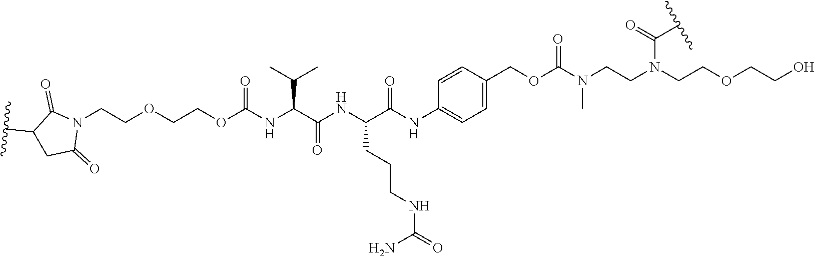 [SMILES: *C(=O)N(CCOCCO)CCN(C)C(=O)OCc1ccc(NC(=O)[C@H](CCCNC(N)=O)NC(=O)[C@@H](NC(=O)OCCOCCN2C(=O)CC(*)C2=O)C(C)C)cc1] | 206 |
| 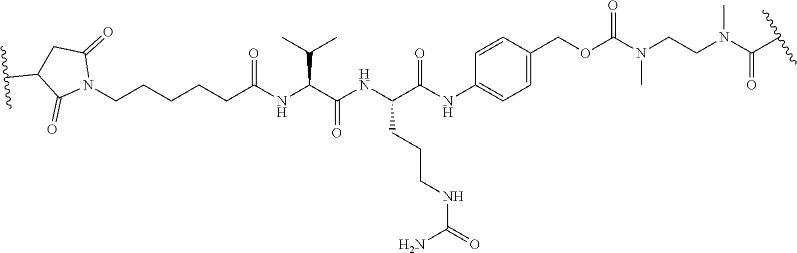 [SMILES: *C(=O)N(C)CCN(C)C(=O)OCc1ccc(NC(=O)[C@H](CCCNC(N)=O)NC(=O)[C@@H](NC(=O)CCCCCN2C(=O)CC(*)C2=O)C(C)C)cc1] | 207 |
| 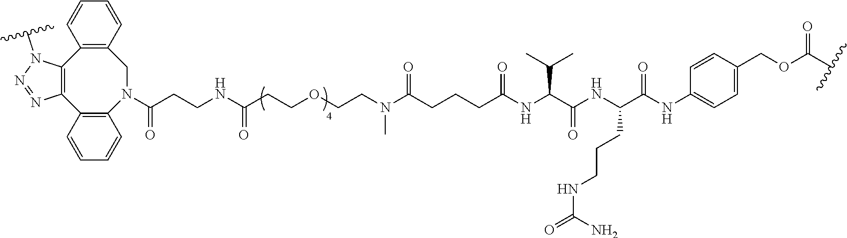 | 208 |
| 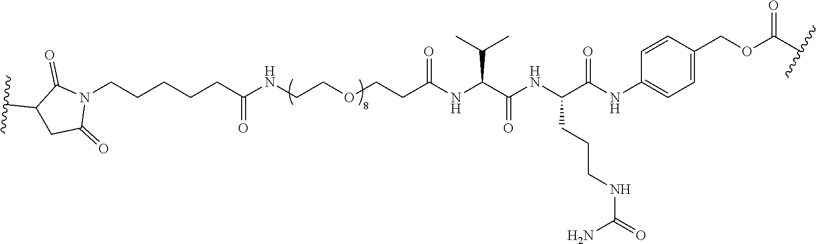 | 209 |
| 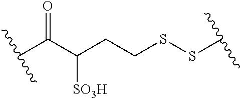 [SMILES: *SSCCC(C(*)=O)S(=O)(=O)O] | 210 |
| 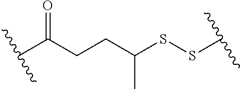 [SMILES: *SSC(C)CCC(*)=O] | 211 |
| 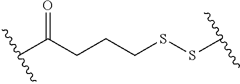 [SMILES: *SSCCCC(*)=O] | 212 |
| 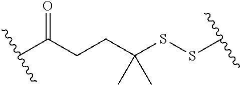 [SMILES: *SSC(C)(C)CCC(*)=O] | 213 |
| 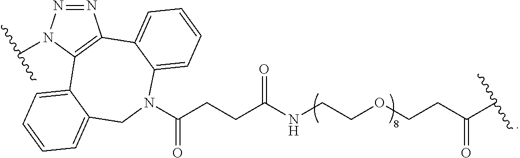 | 214 |

In some embodiments, the linker is a non-cleavable linker.

In some embodiments, each amino acid moiety of —X_{AA}— is independently selected from alanine (Ala), arginine (Arg), glycine (Gly), histidine (His), isoleucine (Ile), leucine (Leu), lysine (Lys), phenylalanine (Phe), tryptophan (Trp), tyrosine (Tyr), valine (Val), citrulline (Cit), and homocitrulline (HoCit). In some embodiments, each amino acid moiety of —X_{AA}— is independently selected from alanine (Ala), glycine (Gly), lysine (Lys), phenylalanine (Phe), valine (Val), and citrulline (Cit). In some embodiments, the amino acid sequence —X_{AA}— is selected from -Val-Cit- (Linker ID NO. 319), -Cit-Val- (Linker ID NO. 320), -Val-Ala- (Linker ID NO. 321), -Ala-Val- (Linker ID NO. 322), -Phe-Lys- (Linker ID NO. 323), -Lys-Phe- (Linker ID NO. 324), -Ala-Ala- (Linker ID NO. 325), -Val-Val- (Linker ID NO. 326), -Gly-Gly- (Linker ID NO. 327), -Ala-Ala-Ala- (Linker ID NO. 328), -Gly-Gly-Gly- (Linker ID NO. 329), -Gly-Gly-Phe-Gly- (Linker ID NO. 330), -Gly-Phe-Gly-Gly- (Linker ID NO. 331), -Gly-Gly-Gly-Phe- (Linker ID NO. 332), -Phe-Gly-Gly-Gly- (Linker ID NO. 333), and -Gly-Gly-Gly-Gly- (Linker ID NO. 334). In some embodiments, the amino acid sequence —X_{AA}— is selected from -Val-Cit- (Linker ID NO. 319), Cit Val (Linker ID NO. 320), Val Ala (Linker ID NO. 321), Ala Val (Linker ID NO. 322), Phe Lys (Linker ID NO. 323), Lys Phe (Linker ID NO. 324), Ala Ala (Linker ID NO. 325), Val Val (Linker ID NO. 326), Gly Gly (Linker ID NO. 327), Ala Ala Ala (Linker ID NO. 328), and Gly Gly Gly (Linker ID NO. 329).

In some embodiments, the payload moiety P is a drug moiety. In some embodiments, the drug moiety is selected from any of the following:

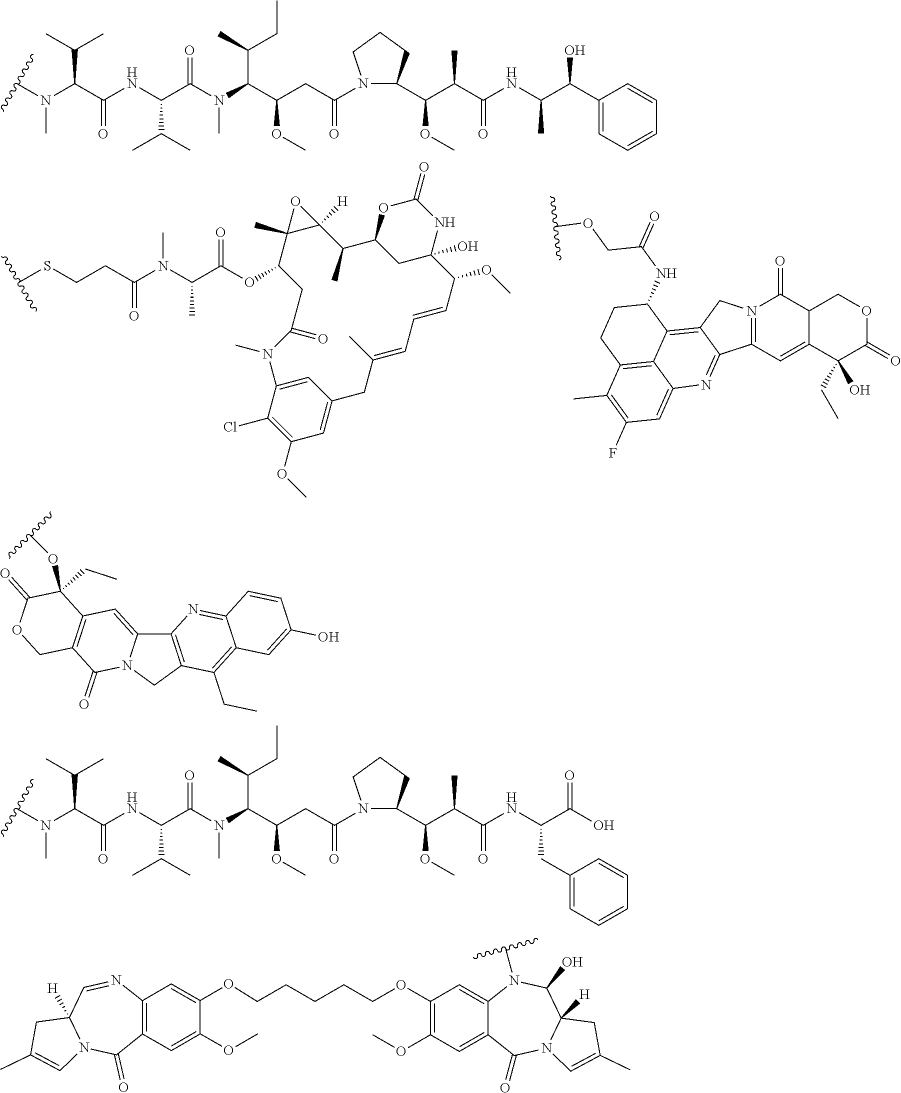

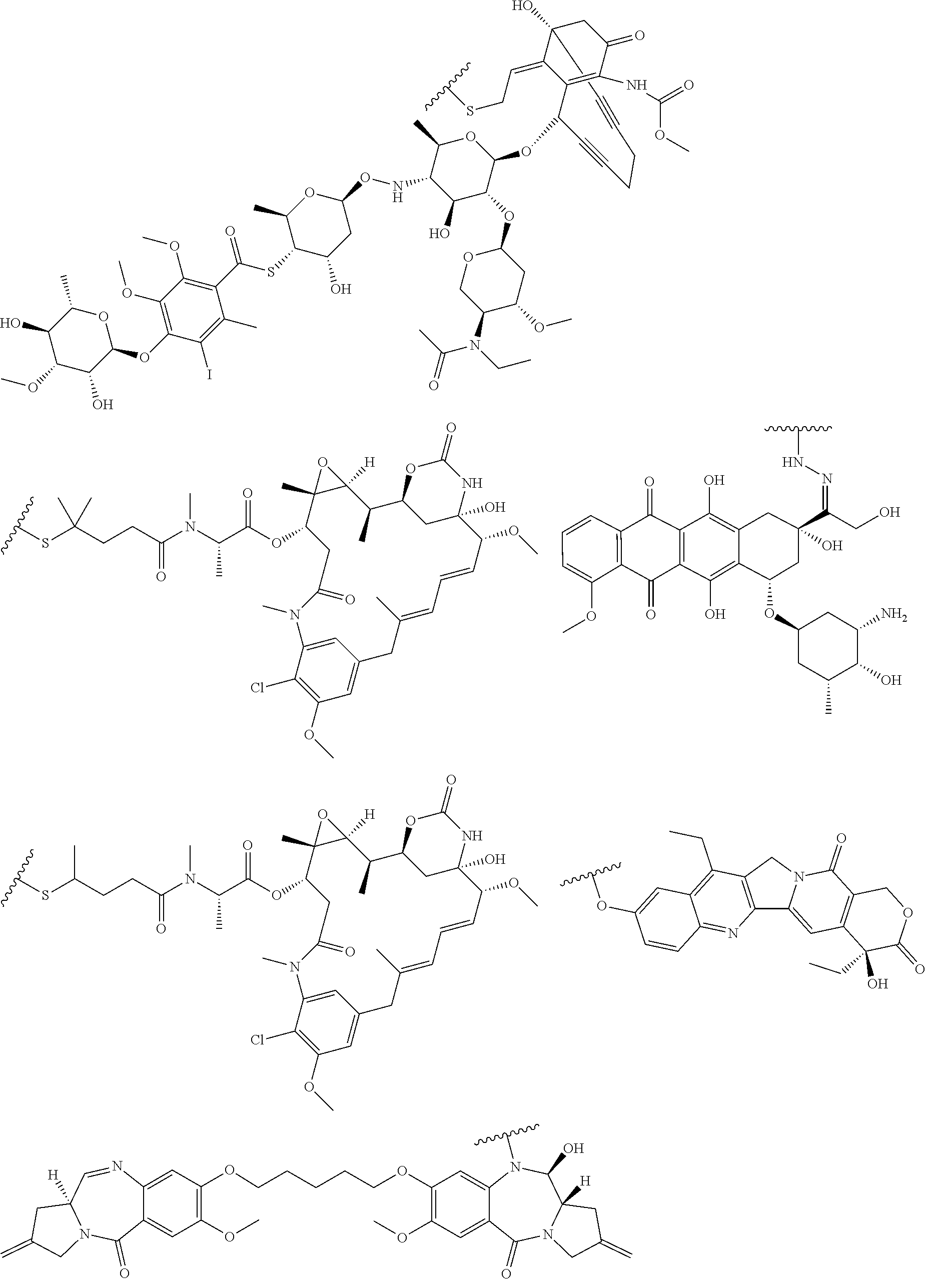

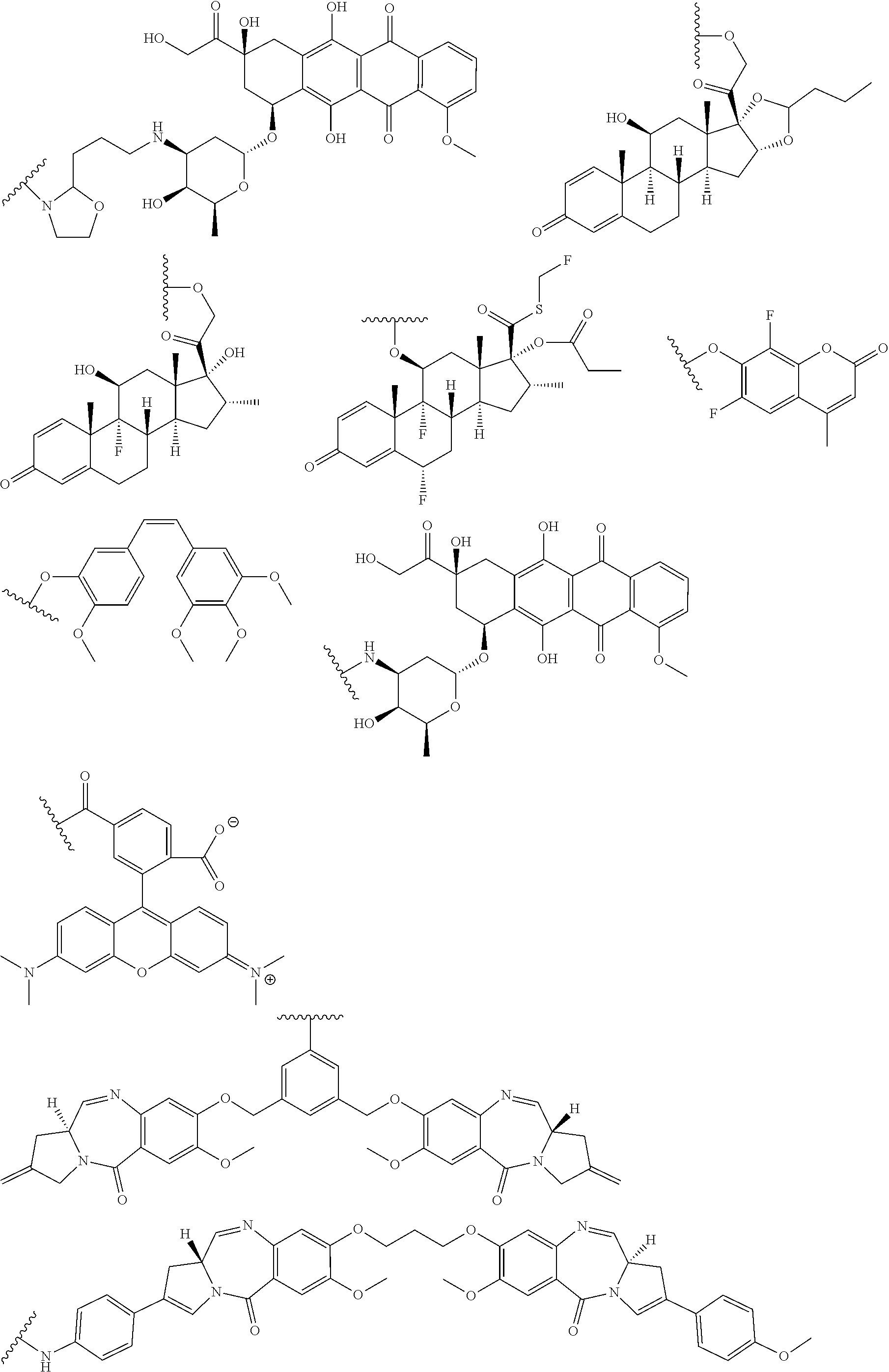

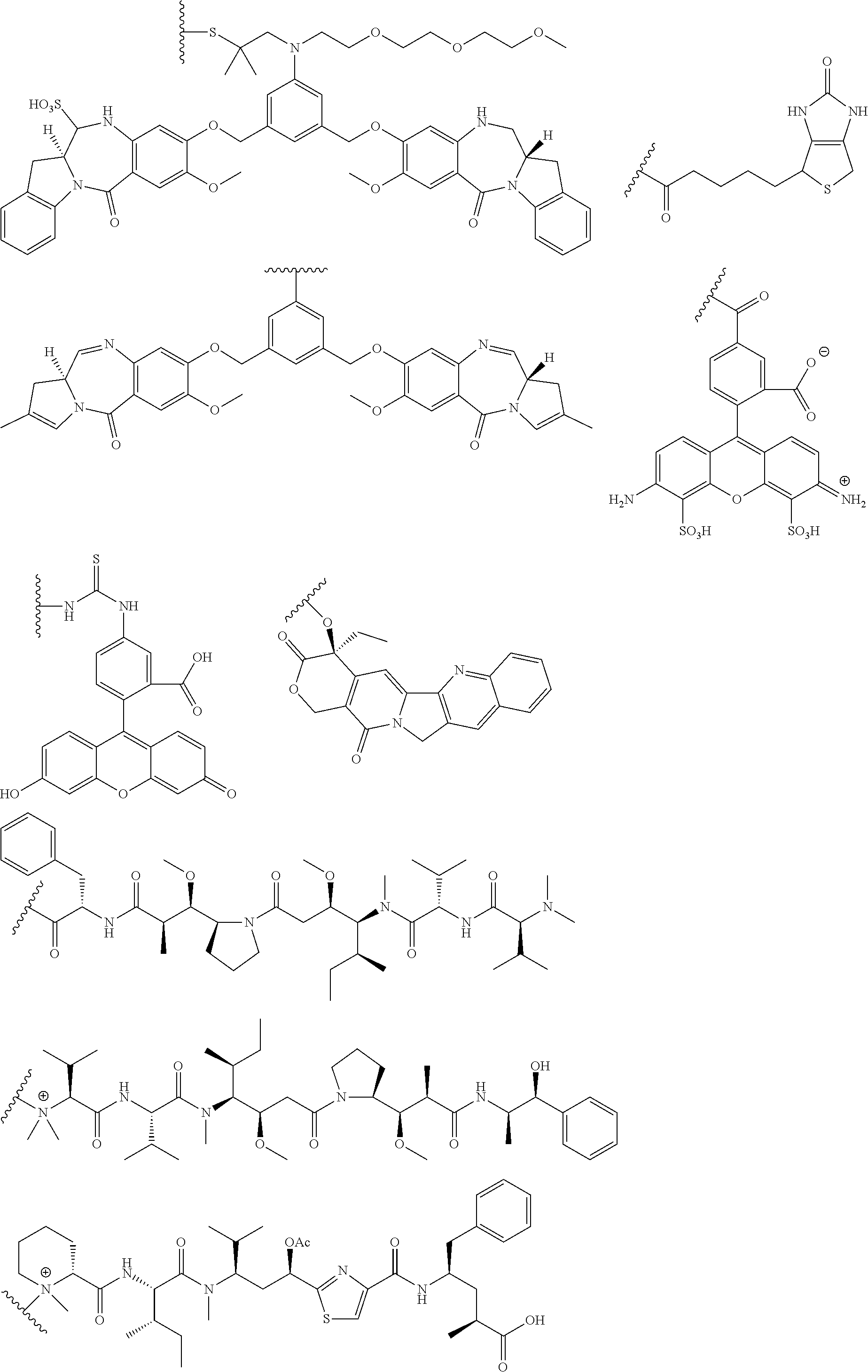

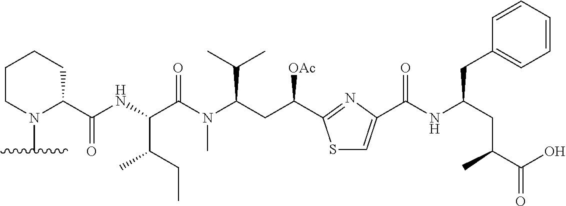 [SMILES: *N1CCCC[C@@H]1C(=O)N[C@H](C(=O)N(C)[C@H](C[C@@H](OC(C)=O)c1nc(C(=O)N[C@@H](Cc2ccccc2)C[C@H](C)C(=O)O)cs1)C(C)C)[C@@H](C)CC]

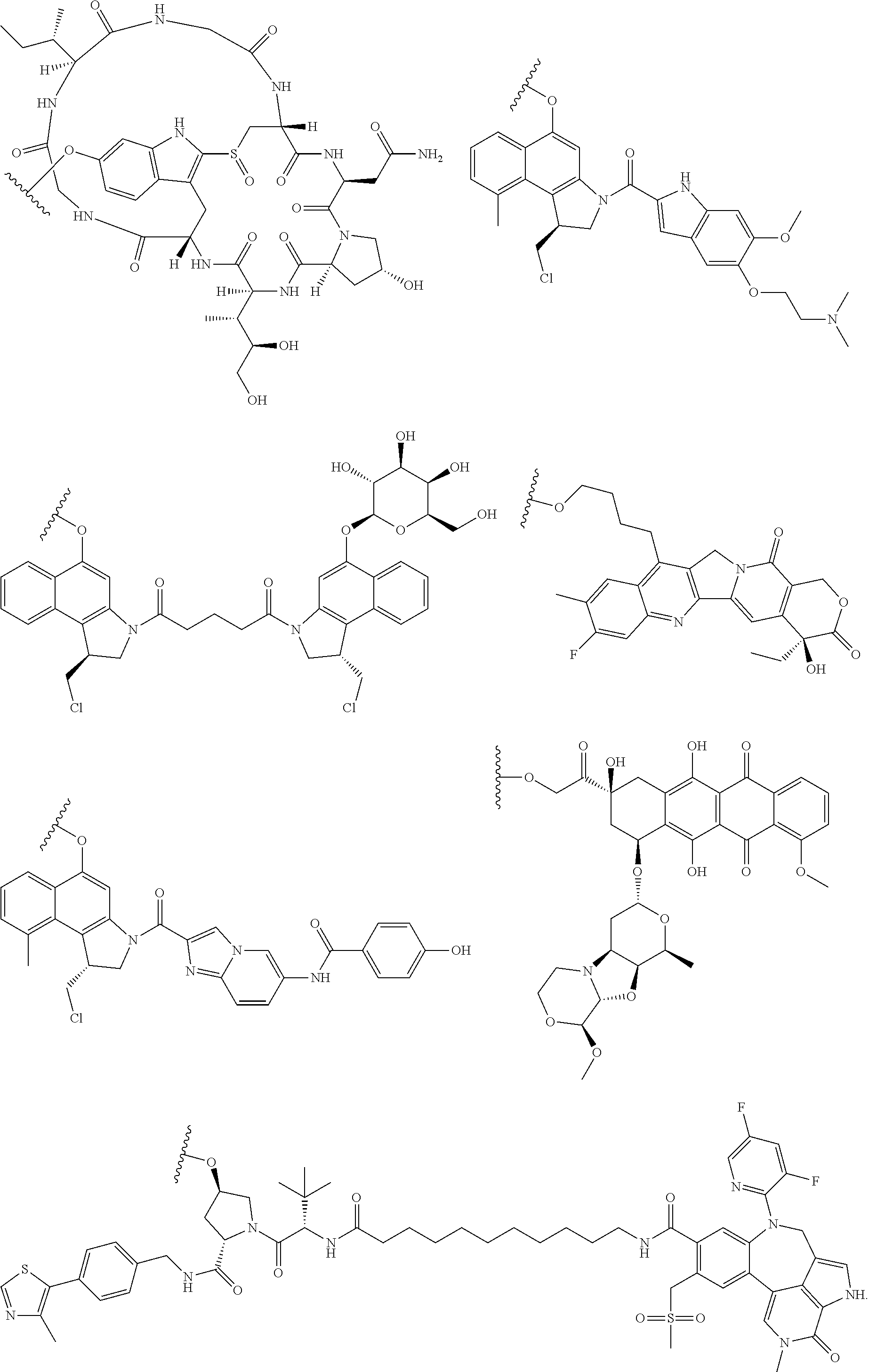

In some embodiments, the VL CDR1 comprises the amino acid sequence of SEQ ID NO:29, CDR2 comprises the amino acid sequence of SEQ ID NO:10, CDR3 comprises SEQ ID NO:11; and the VH CDR1 comprises the amino acid sequence of SEQ ID NO:15, CDR2 comprises the amino acid sequence of SEQ ID NO:26, CDR3 comprises the amino acid sequence of SEQ ID NO:5.

In some embodiments, the antibody, antigen-binding fragment thereof or cell-penetrating fragment thereof comprises a light chain variable region (VL) comprising an amino acid sequence of SEQ ID NO:21 and a heavy chain variable region (VH) comprising an amino acid sequence of SEQ ID NO:14. In some embodiments, the antibody, antigen-binding fragment thereof or cell-penetrating fragment thereof comprises a full length light chain (LC) comprising an amino acid sequence of SEQ ID NO:20 and a full length heavy chain (HC) comprising an amino acid sequence of SEQ ID NO:13.

In some embodiments, the antibody, antigen-binding fragment thereof or cell-penetrating fragment thereof comprises: a light chain variable domain (VL) comprising an amino acid sequence that is at least about 95% identical to an amino acid sequence selected from the group consisting of 3E10-VL-H1 (SEQ ID NO:85), 3E10-VL-H2 (SEQ ID NO:86), 3E10-VL-H3 (SEQ ID NO:87), 3E10-VL-H4 (SEQ ID NO:88), 3E10-VL-H5 (SEQ ID NO:89), and 3E10-VL-H6 (SEQ ID NO:90); and a heavy chain variable domain (V_{H}) comprising an amino acid sequence that is at least about 95% identical to an amino acid sequence selected from the group consisting of 3E10-VH-H1 (SEQ ID NO:64), 3E10-VH-H2 (SEQ ID NO:65), 3E10-VH-H3 (SEQ ID NO:66), 3E10-VH-H4 (SEQ ID NO:67), 3E10-VH-H5 (SEQ ID NO:68), 3E10-VH-H6 (SEQ ID NO:69), and 3E10-VH-H7 (SEQ ID NO:70).

In some embodiments, the antibody, antigen-binding fragment thereof or cell-penetrating fragment thereof comprises: a light chain variable domain (VL) comprising an amino acid sequence selected from the group consisting of 3E10-VL-H1 (SEQ ID NO:85), 3E10-VL-H2 (SEQ ID NO:86), 3E10-VL-H3 (SEQ ID NO:87), 3E10-VL-H4 (SEQ ID NO:88), 3E10-VL-H5 (SEQ ID NO:89), and 3E10-VL-H6 (SEQ ID NO:90); and a heavy chain variable domain (VH) comprising an amino acid sequence selected from the group consisting of 3E10-VH-H1 (SEQ ID NO:64), 3E10-VH-H2 (SEQ ID NO:65), 3E10-VH-H3 (SEQ ID NO:66), 3E10-VH-H4 (SEQ ID NO:67), 3E10-VH-H5 (SEQ ID NO:68), 3E10-VH-H6 (SEQ ID NO:69), and 3E10-VH-H7 (SEQ ID NO:70).

In some embodiments, the antibody, antigen-binding fragment thereof or cell-penetrating fragment thereof comprises a VL/VH pair selected from the group consisting of (a) VL1 (SEQ ID NO:85) and VH1 (SEQ ID NO:64), (b) VL1 (SEQ ID NO:85) and VH2 (SEQ ID NO:65), (c) VL1 (SEQ ID NO:85) and VH3 (SEQ ID NO:66), (d) VL1 (SEQ ID NO:85) and VH4 (SEQ ID NO:67), (e) VL2 (SEQ ID NO:86) and VH1 (SEQ ID NO:64), (f) VL2 (SEQ ID NO:86) and VH2 (SEQ ID NO:65), (g) VL2 (SEQ ID NO:86) and VH3 (SEQ ID NO:66), (h) VL2 (SEQ ID NO:86) and VH4 (SEQ ID NO:67), (i) VL3 (SEQ ID NO:87) and VH1 (SEQ ID NO:64), (j) VL3 (SEQ ID NO:87) and VH2 (SEQ ID NO:65), (k) VL3 (SEQ ID NO:87) and VH3 (SEQ ID NO:66), (l) VL3 (SEQ ID NO:87) and VH4 (SEQ ID NO:67), (m) VL4 (SEQ ID NO:88) and VH1 (SEQ ID NO:64), (n) VL4 (SEQ ID NO:88) and VH2 (SEQ ID NO:65), (o) VL4 (SEQ ID NO:88) and VH3 (SEQ ID NO:66), (p) VL4 (SEQ ID NO:88) and VH4 (SEQ ID NO:67), (q) VL5 (SEQ ID NO:89) and VH5 (SEQ ID NO:68), (r) VL5 (SEQ ID NO:89) and VH6 (SEQ ID NO:69), (s) VL6 (SEQ ID NO:90) and VH5 (SEQ ID NO:68), and (t) VL6 (SEQ ID NO:90) and VH6 (SEQ ID NO:69).

In some embodiments, the antibody, antigen-binding fragment thereof or cell-penetrating fragment thereof comprises: a light chain variable domain (VL) comprising 3E10-VL-H6 (SEQ ID NO:90) and a heavy chain variable domain (VH) comprising 3E10-VH-H6 (SEQ ID NO:69).

In some embodiments, the antibody, antigen-binding fragment thereof or cell-penetrating fragment thereof comprises: a light chain variable domain (VL) comprising the amino acid sequence (DIQMTQSPSSLSASLGDRATITCRASKTVSTSSYSYMHWYQQKPGQPPKLLIKYASYLE SGVPSRFSGSGSGTDFTLTISSLQPEDAATYYCQHSREFPWTFGGGTKVEIK) (SEQ ID NO:126) and a heavy chain variable domain (VH) comprising the amino acid sequence (EVQLVESGGGLVQPGGSLRLSCAASGFTFSNYGMHWVRQAPGKGLEWVSYISSGSSTI YYADSVKGRFTISRDNAKNSLYLQMNSLRAEDTAVYYCARRGLLLDYWGQGTTVTVS S) (SEQ ID NO:127).

In embodiments, the present disclosure provides a method for treating a subject in need thereof, the method comprising administering a therapeutically effective amount of a conjugate of the disclosure to the subject.

### BRIEF DESCRIPTION OF THE FIGURES

FIGS. 1A-1B show a conjugation strategy for preparing an antibody-drug conjugate (ADC) according to an aspect of the disclosure. FIG. 1A is a schematic showing the steps for labeling a 3E10 antibody, such as a chimeric 3E10-cD31N antibody (cD31N) with a succinimidyl 4-(N-maleimidomethyl)cyclohexane-1-carboxylate (to an SMCC) linker (step 1, SMCC labeling), and subsequently conjugating the Maytansinoid DM1 linker to the warhead (N2′-deacetyl-N2′-(3-mercapto-1-oxopropyl)-maytansine) (DM1) (step 2, DM1 labeling). FIG. 1B provides further details on an ADC synthesis.

FIG. 2 is a high performance liquid chromatography (HPLC) chromatogram measuring the quantity and size of DM1, cD31N (the 3E10-cD31N antibody) and the cD31N-DM1 conjugate following 14 days of incubation at 4° C.

FIG. 3 shows the viability of B16 melanoma cells treated with different concentrations of DM1 alone, or cD31N-DM1 conjugate after three days.

FIG. 4 shows the viability of Capan-1 Pancreatic Cancer cells treated with different concentrations of DM1 alone or cD31N-DM1 conjugate after six days.

FIG. 5 shows the viability of DAYO Medulloblastoma cells treated with different concentrations of DM1 alone or cD31N-DM1 conjugate after three days.

FIG. 6 shows the viability of GL261 Glioma cells treated with different concentrations of DM1 alone or cD31N-DM1 conjugate after six days.

FIG. 7 shows the viability of wild-type BRCA2 (WT or BRCA2^{wt/wt}) (FIG. 7A) and BRCA2 deficient (BRCA2^{-/-}) (FIG. 7B) DLD-1 colorectal adenocarcinoma cells treated with different concentrations of DM1 alone or cD31N-DM1 conjugate after 6 days.

FIG. 8 shows the viability of PEO-4 (WT) (FIG. 8A) and PEO-1 (BRCA2^{+/+}) (FIG. 8B) ovarian adenocarcinoma cells treated with different concentrations of DM1 alone or cD31N-DM1 conjugate after 5 days.

FIG. 9 is a schematic showing the conjugation strategy for preparing an antibody-drug conjugate according to an aspect of the disclosure. Conjugation of a 3E10 antibody to an O-Succinyl linker and the anti-tumor drug SN38 is shown.

FIG. 10 is a Western Blot showing the results of treatment of WT and BRCA2^{-/-} DLD-1 cells with different concentrations of SN38 alone, wild-type 3E10 chimeric antibody (cWT), cWT-SN38 conjugate, cD31N antibody, or cD31N-SN38 conjugate. The protein gamma (γ)-H2AX is a marker of DNA damage.

FIG. 11 shows the viability of WT (FIG. 11A) and BRCA2^{-/-} (FIG. 11B) DLD-1 treated with different concentrations of SN38 alone, cWT-SN38 conjugate, or cD31N-SN38 conjugate.

FIG. 12 shows the results of treatment of an in vivo mouse model of melanoma with cD31N-SN38 conjugate. FIG. 12A shows tumor growth over time after mice were treated with 12.5 mg/kg of the cD31N-SN38 conjugate (n=4) or phosphate buffered saline (PBS) control (n=10). FIG. 12B shows the weight of mice over time post-implantation treated with the cD31N-SN38 conjugate, as compared with mice treated with PBS.

FIG. 13 shows a timeline of treatment duration experiments described herein.

FIGS. 14, 15, and 16 show the cell viability of WT and BRCA2^{-/-} DLD-1 cells after treatment with SN38, cWT-SN38 or cD31N-SN38 at 3, 5, and 7 days post-treatment, respectively.

FIGS. 14A, 15A, and 16A show the viability of WT DLD-1 cells and FIGS. 14B, 15B, and 16B show the viability of BRCA2^{-/-}- DLD-1 cells. The calculated EC₅₀ values are provided.

FIGS. 17 and 18 show the effects of shorter and longer term treatment on cell viability in WT DLD-1 cells (FIGS. 17A and 17B) and BRCA2^{-/-}- DLD-1 cells (FIGS. 18A and 18B) at day 7 after treatment with SN38 or cD31N-SN38 conjugate. Shorter term treatment was defined as a 24 hour treatment followed by a change to fresh treatment-free media. Longer term treatment was defined as 7 days of treatment without a change of medium. The calculated EC₅₀ values are provided.

FIG. 19 shows the results of clonogenic survival assays in WT (FIG. 19A) and BRCA2^{-/-}- (FIG. 19B) DLD-1 cells after treatment with different concentrations of SN38, cWT antibody or cWT-SN38 conjugate. Data is shown as % survival fraction normalized to No Treatment (NT).

FIG. 20 shows the results of clonogenic survival assays in WT (FIG. 20A) and BRCA2^{-/-}- (FIG. 20B) DLD-1 cells after treatment with different concentrations of SN38, cD31N antibody or cD31N-SN38 conjugate. Data is shown as % survival fraction normalized to No Treatment (NT).

FIGS. 21-22 are Western blots detecting specific DNA damage response proteins in WT and BRCA2^{-/-} DLD-1 cells after treatment with SN38, cWT antibody, cD31N antibody, cWT-SN38 conjugate, Or cD31N-SN38 conjugate. Results are shown in DLD-1 cells collected after 24 hours (FIG. 21A), 48 hours (FIG. 21B), and 96 hours (FIG. 22) after treatment.

FIGS. 23-24 show cell viability of WT and IDH1^{mut} HCT116 colorectal cancer cells after treatment with different concentrations of SN38, cD31N antibody, or cD31N-SN38. FIG. 23 shows the viability of WT (FIG. 23A) and IDH1^{mut} (FIG. 23B) HCT116 cells at day 5 of treatment.

FIG. 24 shows the viability of WT (FIG. 24A) and IDH1^{mut} (FIG. 24B) HCT116 cells at day 7 of treatment. The calculated EC₅₀ values are provided.

FIG. 25 shows the results of clonogenic survival assays in WT (FIG. 25A) and IDH1^{mut} (FIG. 25B) HCT116 cells after treatment with different concentrations of SN38, cD31N antibody or cD31N-SN38 conjugate. Data is shown as % survival fraction normalized to No Treatment (NT).

FIG. 26 is a Western blot of DNA damage response proteins in WT and IDH1^{mut} HCT116 cells after treatment with 100 nM of SN38, humanized 3E10 antibody V66, or V66-SN38 conjugate.

FIG. 27 shows the viability of LoVo colorectal carcinoma cells treated with SN38, cD31N antibody, cD31N Fab antibody fragment, cD31N-SN38 conjugate, cDN31-Fab-SN38 conjugate, V66 antibody, V66 Fab antibody fragment, V66-SN38 conjugate, and the V66-Fab-SN38 conjugate.

FIG. 28 shows the viability of SKOV3 ovarian cancer cells after 7 days of treatment with different concentrations of SN38, cD31N antibody, or cD31N-SN38 conjugate. The calculated EC₅₀ values are provided.

FIG. 29 shows the results of treatment on SKOV3 ovarian cancer cells treated with SN38, cD31N antibody, cD31N Fab antibody fragment, cD31N-SN38 conjugate, cDN31-Fab-SN38 conjugate, V66 antibody, V66 Fab antibody fragment, V66-SN38 conjugate, V66-Fab-SN38 conjugate, ScFv cD31N antibody, and ScFv 3E10 wild type antibody.

FIG. 30 is a Western blot of DNA damage response proteins in SKOV3 ovarian cancer cells after treatment with 100 nM of SN38, cD31N-SN38 conjugate, and 3E10-cD31N antibody at 24 hours, 48 hours, and 72 hours.

FIG. 31 shows the cell viability of PEO-4 and PEO-1 ovarian cancer cells at day 7 after longer term treatment with different concentrations of SN38, cD31N antibody, or cD31N conjugate. FIG. 31A shows cell viability in PEO-1 cells and FIG. 31B shows cell viability in PEO-4 cells. The calculated EC₅₀ values are provided.

FIG. 32 shows a Western blot of DNA damage response proteins in PEO-4 and PEO-1 cells after treatment with 100 nM of SN38, cD31N antibody or cD31N-SN38 conjugate. FIG. 32A shows protein levels after 24 hours of treatment and FIG. 32B shows protein levels after 48 hours of treatment.

FIG. 33 shows the results of an in vivo mouse model of colorectal cancer treated with systemic administration of a cD31N-SN38 conjugate for four consecutive days, followed by two days without treatment, or for two consecutive days followed by five days without treatment. FIG. 33A shows tumor growth over time after mice were treated with 12.5 mg/kg of the cD31N-SN38 conjugate or phosphate buffered saline (PBS) control. FIG. 33B shows the weight of mice over time post-implantation treated with the cD31N-SN38 conjugate, as compared with mice treated with PBS.

FIG. 34 shows the in vitro potency of a 3E10-SN38 ADC against different cancer cell lines at nanomolar concentrations.

FIG. 35 shows in vitro potency of a 3E10-DM1 ADC against different cancer cell lines at picomolar concentrations.

FIGS. 36A-36C shows potency of the cD31N-drug conjugate (10 mg/kg) in vivo using a mouse cancer model.

FIG. 37 shows the viability of human Dukes-type C colorectal adenocarcinoma (DLD1) cell line treated with V66-PNU, V66-MMAE, and V66-SN38 conjugates.

FIG. 38A-FIG. 38C shows the results of vitro potency of3E10 ADCs against 3 ENT2(+) tumor cell lines (MCF7, LOVO, and A427).

FIG. 39A shows the Lys-azide conjugation intermediates of 3E10-D31N monoclonal antibody (V66) after proteolysis.

FIG. 39B shows the mass spectroscopy results of mapped Lys-azide conjugation intermediates of 3E10-D31N monoclonal antibody (V66) after proteolysis.

FIG. 39C shows the sequence of humanized 3E10-D31N monoclonal antibody (V66) and 3E10-D31N.

FIGS. 40A and 40B collectively illustrate improved cellular internalization of 3E10-D31N monoclonal antibody (V66) oligonucleotide conjugates in A427 cells utilizing transglutaminase-mediated enzymatic conjugation.

FIG. 41A shows tumor volume in mice treated with V66-MMAE conjugate with a schedule of QWx4, 2QWx4, or 3QWx4.

FIG. 41B shows body weight in mice treated with V66-MMAE conjugate with a schedule of QWx4, 2QWx4, or 3QWx4.

FIGS. 42A, 42B, 42C, and 42D collectively show tumor volume in mice treated with V66-MMAE conjugate with a schedule of QWx4, 2QWx4, or 3QWx4.

FIGS. 43A and 43B shows the results of vitro potency of 3E10 ADCs against 3 ENT2(+) tumor cell lines (MCF7, LOVO, and A427).

FIG. 44 shows the structure of a V66- vc-Seco-DUBA ADC.

### DETAILED DESCRIPTION

### I. Introduction

Advantageously, methods and compositions were developed for targeting therapeutic agents, e.g., drugs such as anti-tumor drug, or oligonucleotides, to various cancer tissues in vivo and facilitating delivery of these therapeutic polynucleotides into diseased cells, e.g., cancer cells displaying high levels of ENT2 on their cell surface. Thus, the present disclosure provides compositions, conjugates, and methods for delivering therapeutic agents to cancerous tissue. In some embodiments, the methods and compositions find particular use for the treatment of cancers. For instance, compositions comprising a conjugate of (i) a 3E10 antibody or antigen-binding fragment or variant thereof, and (ii) a therapeutic agent, as well as methods for using such compositions for the treatment of cancers, are described.

The studies described herein demonstrate that ADCs comprising a cell-penetrating and nucleic acid-binding 3E10 antibody conjugated via a linker to an anti-tumor drug or a oligonucleotide can effectively penetrate and kill tumor cells with specificity. For example, Example 3 demonstrates the broad applicability and potency of a cD31N-DM1 conjugate against various cancer cell lines. The cD31N-DM1 ADC was effective in all tested cancer cell lines, regardless of genetic background or tissue of origin and the ADC-drug was more potent than free drug in culture in each cell line. This suggests that targeting of the 3E10 ADC is not limited to a specific cancer antigen or tumor epitope. Advantageously, the 3E10 ADC provided herein can widely target any cancer based on cell surface expression of ENT2, a nucleoside transporter overexpressed in many cancers. Example 5 demonstrates that 3E10-SN38 conjugates can effectively kill colorectal cancer cells by inducing DNA damage via delivery of SN38, Example 6 demonstrates that administration of the cD31N-SN38 conjugate reduces tumor growth in mice without evidence of non-specific toxicity, Example 7 demonstrates that a single dose of SN38 or 3E10-SN38 conjugates can have long-term effects on cancer cell growth and that these effects are dose dependent, and Example 20 demonstrates that systemic administration of the cD31N-SN38 conjugate post implantation reduces tumor growth in mice without evidence of non-specific toxicity. Examples 21 and 22 demonstrate the broad applicability and potency of the cD31N-SN38 conjugate and of the cD31N-DM1 conjugate against various cancer cell lines, respectively. Example 24 demonstrates that each of the 3E10-D31N monoclonal antibody (V66)-PNU, V66-MMAE, and V66-SN38 conjugates can effectively kill a DLD1 (BRCA2−/−) cancer cell line 7 days post-treatment. Example 27 demonstrates that cellular internalization of V66 oligonucleotide conjugates can be improved using transglutaminase-mediated enzymatic conjugation.

### II. Definitions

The terminology used in the present disclosure is for the purpose of describing particular aspects only and is not intended to be limiting.

As used herein, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. Unless the context requires otherwise, it will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.” Additionally, where the terms “comprising,” “including,” “includes,” “having,” “has,” “with,” or variants thereof are used in either the detailed description and/or the claims, alternatives reciting “consisting of” or “consisting essentially of” are intended to be encompassed within such disclosures.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%.

As used herein, the term “antibody” refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. The term “antibody,” as used herein, is used in the broadest sense and encompasses monoclonal antibodies (including full length monoclonal antibodies), polyclonal antibodies, multispecific antibodies (e.g., bispecific antibodies), chimeric antibodies, humanized antibodies, human antibodies, fusion proteins comprising an antigen determination portion of an antibody, and antibody fragments (such as Fab, Fab′, F(ab′)2, Fv fragments, scFv molecules), and any other modified immunoglobulin molecule comprising an antigen recognition site, so long as they exhibit one or more of the desired biological activities. In embodiments, “desired biological activity” of an antibody refers to the ability of the antibody to bind to its target antigen, e.g., a nucleic acid, e.g., DNA. In embodiments, “desired biological activity” can further include antibody binding to its target antigen and resulting in a measurable biological response which can be measured in vitro or in vivo. Such activity can be antagonistic or agonistic. In embodiments, “desired biological activity” of an antibody refers to the ability of the antibody to bind to a target, e.g., nucleic acid molecules. In embodiments, “desired biological activity” of an antibody refers to the ability of the antibody to bind to a cellular receptor, e.g., ENT2. In embodiments, “desired biological activity” of an antibody refers to the ability of the antibody to be internalized by a target cell. “Target antigen,” as used herein, refers to the molecule that is bound specifically by the antigen-binding domain comprising the variable regions of a given antibody. The term “specifically binds” refers to the binding of an antibody to its cognate antigen (e.g., a nucleic acid, e.g., DNA) while not significantly binding to other antigens.

Depending on the amino acid sequences of the constant domains of their heavy chains, antibodies (immunoglobulins) can be assigned to different classes. There are five major classes of immunoglobulins: IgA, IgD, IgE, Ig1, and IgM, and several of these can be further divided into subclasses or isotypes, e.g., Ig1₁, Ig1₂, Ig1₃, Ig1₄, IgA₁, and IgA₂. “Isotype,” as used herein, refers to any of the subclasses of immunoglobulins defined by the chemical and antigenic characteristics of their constant regions. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4^{th} ed. (W.B. Saunders, Co., 2000). It should be understood that antibodies disclosed herein can also comprise hybrids of isotypes and/or subclasses.

Antibodies of the present disclosure are generally isolated or recombinant. “Isolated,” when used to describe the various polypeptides disclosed herein, refers to a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. As used herein, “recombinant antibody” refers to an antibody that is generated using recombinant nucleic acid techniques in exogenous host cells, and recombinant antibodies can be isolated as well.

“Native antibodies” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen-binding site. The constant domain contains the CH1, CH2 and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) or complementary determining regions (CDRs), both in the light-chain and the heavy-chain variable domains, that confer antigen specificity. A “variable heavy domain” pairs with a “variable light domain” to form an antigen-binding domain (ABD) that specifically binds a target antigen. The more highly conserved portions of variable domains are called the framework regions (FR). The variable domains of native heavy and light chains each comprise four FR regions, largely adopting a beta-sheet configuration, connected by three CDRs/HVRs, which form loops connecting, and in some cases forming part of, the beta-sheet structure. The CDRs/HVRs in each chain are held together in close proximity by the FR regions and, with the CDRs/HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The terms “hypervariable region,” “HVR,” “HV,” “complementary determining region,” and “CDR,” used interchangeably herein, refer to the regions of an antibody variable domain which are hypervariable in sequence and/or form structurally defined loops. Generally, antibodies comprise six HVRs or CDRs; three in the VH (H1, H2, H3; or VH CDR1, VH CDR2, VH CDR3), and three in the VL (L1, L2, L3; or VL CDR1, VL CDR2, VL CDR3).

The “light chains” of antibodies (immunoglobulins) from any mammalian species can be assigned to one of two clearly distinct types, called kappa (“κ”) and lambda (“λ”), based on the amino acid sequences of their constant domains.

Together, the CDRs of the VH and VL domains form an Fv region. In embodiments, a VH and a VL domain comprise the six CDRs of the ABD. In a “Fab” format, the variable heavy domain (VH; containing VH CDR1, VH CDR2, and VH CDR3) and the variable light domain (VL or VL; containing the VL CDR1, VL CDR2 and VL CDR3), comprise the set of 6 CDRs, with the C-terminus of the VH domain being attached to the N-terminus of the CHI domain of the heavy chain and the C-terminus of the VL domain being attached to the N-terminus of the constant light domain (and thus forming the light chain). In an “scFv” format, the VH and VL domains are covalently attached, generally through the use of a linker (e.g., an “scFv linker”), into a single polypeptide sequence, which can have the N- to C-terminus arrangement of VH-linker-VL or VL-linker-VH. In general, the C-terminus of the scFv domain is attached to the N-terminus of the hinge in the second monomer.

“Fab” or “Fab region,” as used herein, refers to a polypeptide that comprises VH, CH1, VL, and CL immunoglobulin domains, generally on two different polypeptide chains (e.g., VH-CH1 on one chain and VL-CL on the other). Fab can refer to this region in isolation, or this region in the context of an antibody of the disclosure. In embodiments, a Fab comprises an Fv region in addition to CH1 CL domains.

Another part of the heavy chain is the hinge region. As used herein, “hinge,” “hinge region,” “antibody hinge region,” or “hinge domain” refers to the flexible polypeptide comprising the amino acids between the first and second constant domains of an antibody. Structurally, the IgG CH1 domain ends at EU position 215, and the IgG CH₂ domain begins at residue EU position 231. Thus, for IgG, the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain.

“Heavy chain constant region,” as used herein, refers to the CH1-hinge-CH2-CH3 portion of an antibody or fragment thereof, excluding the variable heavy domain. In embodiments, the heavy chain constant region comprises amino acids 118-447 of human IgG1, in EU numbering. As used herein, “heavy chain constant region fragment” refers to a heavy chain constant region that contains fewer amino acids from either or both of the N- and C-termini but still retains the ability to form a dimer with another heavy chain constant region.

“Fv,” “Fv fragment,” or “Fv region,” as used herein, refers to a polypeptide that comprises VL and VH domains of an antibody binding domain. Fv regions can be formatted as both Fabs and scFvs, where the VL and VH domains are combined (e.g., by way of a linker, as discussed herein) to form an scFv.

“Fc,” “Fc region,” or “Fc domain,” as used herein, refers to a polypeptide comprising CH2-CH3 domains of an IgG molecule, and, in some cases, inclusive of the hinge. In EU numbering for human IgG1, the CH2-CH3 domain comprises amino acids 231 to 447, and the hinge is 216 to 230. Thus, the definition of “Fc domain” includes both amino acids 231-447 (CH2-CH3) and 216-447 (hinge-CH2-CH3) of IgG1, or fragments thereof. An “Fc fragment” in this context can contain fewer amino acids from either or both of the N- and C-termini but still retains the ability to form a dimer with another Fc domain or Fc fragment as can be detected using standard methods, generally based on size (e.g., non-denaturing chromatography, size exclusion chromatography, etc.). In embodiments, the disclosed ADCs comprise human Fc domains. In embodiments, the disclosed ADCs comprise Fc domains from human IgG1, Ig12, or IgG4.

A “variant Fc domain” contains amino acid modifications as compared to a parental Fc domain. Thus, a “variant human IgG1 Fc domain” is one that contains amino acid modifications (generally amino acid substitutions, although in the case of ablation variants, amino acid deletions are included) as compared to the human IgG1 Fc domain. In embodiments, variant Fc domains have at least about 80%, at least about 85%, at least about 90%, at least about 95%, at least about 97%, at least about 98%, or at least about 99% identity to the corresponding parental human IgG Fc domain. In embodiments, the percent identity is calculated using the identity algorithms discussed below. In embodiments, the percent identity is calculated using the BLAST algorithm known in the art, using default parameters. In embodiments, variant Fc domains have from 1 to about 20 (e.g., 1, 2, 3, 4, 5, 6, 7, 8, 9, 10, 11, 12, 13, 14, 15, 16, 17, 18, 19 or 20) amino acid modifications as compared to the parental Fc domain. In embodiments, variant Fc domains retain the ability to form a dimer with Ir Fc domain as measured using known techniques as described herein, such as non-denaturing gel electrophoresis.

For all positions discussed in the present disclosure that relate to antibodies, unless otherwise noted, amino acid position numbering is according to the EU index. The EU index or EU index as in Kabat or EU numbering scheme refers to the numbering of the EU antibody. Kabat et al. collected numerous primary sequences of the variable regions of heavy chains and light chains. Based on the degree of conservation of the sequences, they classified individual primary sequences into the CDR and the framework and made a list thereof. See, SEQUENCES OF IMMUNOLOGICAL INTEREST, 5^{th} edition, NIH publication, No. 91-3242, E. A. Kabat et al.; Edelman et al., 1969, Proc Natl Acad Sci USA 63:78-85, the contents of which are incorporated herein by reference. In embodiments of the present disclosure, amino acid position numbering is according to the IMGT system.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

A “naked antibody” for the purposes herein is an antibody that is not conjugated to a cytotoxic moiety or radiolabel.

The term “monoclonal antibody” as used herein refers to an antibody obtained from a population of substantially homogeneous antibodies, e.g., the individual antibodies comprising the population are identical except for possible mutations, e.g., naturally occurring mutations, that can be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

Antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit one or more of the desired biological activities (see, e.g., U.S. Pat. No. 4,816,567; and Morrison et al., Proc. Natl. Acad. Sci. USA 81:6851-6855 (1984)). Typically, the variable region of both light and heavy chains corresponds to the variable region of antibodies derived from one species of mammals (e.g., mouse, rat, rabbit, etc.) with the desired specificity, affinity, and/or capability, while the constant regions are homologous to the sequences of antibodies derived from another species of mammals (e.g., human) to avoid eliciting an immune response In that species. Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In embodiments, a humanized antibody is a human immunoglobulin (recipient antibody) in which residues from a CDR/HVR of the recipient are replaced by residues from a CDR/HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies can comprise residues that are not found in the recipient antibody or in the donor antibody. These modifications can be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. See, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. Examples of methods used to generate humanized antibodies are described in U.S. Pat. No. 5,225,539 or 5,639,641, incorporated herein by reference in their entireties.

As used herein, the term “human antibody” refers to an antibody which possesses an amino acid sequence which corresponds to that of an antibody produced by a human and/or has been made using any technique known in the art. This definition of a human antibody includes intact or full-length antibodies, fragments thereof, and/or antibodies comprising at least one human heavy and/or light chain polypeptide. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10⁻⁷M, preferably no more than about 1×10⁻⁸M and preferably no more than about 1×10⁻⁹ M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

The expression “linear antibodies” refers to the antibodies described in Zapata et al. (1995 Protein Eng, 8(10):1057-1062). Briefly, these antibodies comprise a pair of tandem Fd segments (VH-CH1-VH-CH1) which, together with complementary light chain polypeptides, form a pair of antigen-binding regions. Linear antibodies can be bispecific or monospecific.

“Modification,” as used herein, refers to an amino acid substitution, insertion, deletion, and/or any other mutation in a polypeptide sequence.

“Variant protein,” or “protein variant,” or “variant,” as used herein refers to a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., described herein, are generally at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% identical to the parent polypeptide, using any alignment program known in the art, such as BLAST.

Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol.48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see the webpage located at URL blast.ncbi.nlm.nih.gov/Blast.cgi. When using any of the aforementioned algorithms, the default parameters (for Window length, gap penalty, etc.) are used. Unless specifically stated otherwise, sequence identity is determined using the BLAST algorithm, using default parameters.

In embodiments, a parent polypeptide, for example an Fc parent polypeptide, is a human wild type sequence, such as the heavy constant domain or Fc region from IgGI, Ig12, Ig13 or IgG4, although human sequences with variants can also serve as “parent polypeptides.” In embodiments, antibody sequences described herein have at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity with a parent polypeptide sequence. Accordingly, “antibody variant” or “variant antibody” as used herein refers to an antibody that differs from a parent antibody by virtue of at least one amino acid modification; “Ig1 variant” or “variant IgG” as used herein refers to an IgG that differs from a parent IgG (e.g., from a human IgG sequence) by virtue of at least one amino acid modification; “immunoglobulin variant” or “variant immunoglobulin” as used herein refers to an immunoglobulin sequence that differs from that of a parent immunoglobulin sequence by virtue of at least one amino acid modification; and “Fc variant” or “variant Fc” as used herein refers to an Fc that differs from a parent Fc, e.g., an Fc domain of human IgG1, Ig12, Ig13, or IgG4, by virtue of at least one amino acid modification.

“Ig1 subclass modification” or “isotype modification,” as used herein, refers to amino acid modifications that convert one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

“Non-naturally occurring modification” as used herein is meant an amino acid modification that is not isotypic. For example, because none of the human IgGs comprise a serine at position 434, the substitution 434S in IgG1, Ig12, Ig13, or IgG4 (or hybrids thereof) is considered a non-naturally occurring modification.

As used herein, “oligonucleotide” or “polynucleotide,” used interchangeably, refers to a linear polymer of natural or modified nucleoside monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides and polynucleotides can be natural or synthetic. Oligonucleotides and polynucleotides can include deoxyribonucleosides, ribonucleosides, and/or non-natural analogs thereof. In embodiments, oligonucleotides or polynucleotides are capable of specifically binding to a target genome by way of a regular pattern of monomer-to-monomer interactions, such as Watson-Crick type of base pairing, base stacking, Hoogsteen or reverse Hoogsteen types of base pairing, or the like.

As used herein, a “3E10 antibody” refers to an antibody with a set of heavy chain CDRs (VH CDR1, VH CDR2, and VH CDR3), identified according to the Kabat system, comprising amino acid sequences that vary from SEQ ID NOS: 58, 59, and 60 by no more than two amino acids each, respectively, a set of light chain CDRs (VL CDR1, VL CDR2, and VL CRD3) comprising amino acid sequences that vary from SEQ ID NOS: 61, 62, and 63 by no more than two amino acids each, respectively, that binds nucleic acids and is cell-penetrating at least when bound to a nucleic acid. As described herein, the 3E10 antigen is a polynucleotide.

As used herein, the term “cell-penetrating” refers to an antibody or antigen binding fragment thereof that can penetrate a cell, e.g., a mammalian cell, without the aid of an exogeneous transport vehicle, such as a liposome, or a conjugated cell-penetrating peptide. With respect to 3E10 antibodies and antigen binding fragments thereof, the cell-penetrating antibody or antigen binding fragment thereof can penetrate a cell expressing an ENT2 receptor on its cell surface in the presence of nucleic acids, e.g., non-covalently bound and/or conjugated to the 3E10 antibody or antigen binding fragment thereof, resulting in internalization of the 3E10 antibodies and antigen binding fragments thereof. In some embodiments, the cell-penetrating 3E10 antibody or antigen binding fragment thereof is conjugated to a functional molecule, e.g., a chemical agent, polynucleotide, or polypeptide. Although the cell-penetrating molecules are generally referred to herein as “cell-penetrating antibodies,” it will be appreciated that fragments, including antigen-binding fragments, variants, binding proteins and fusion proteins such as scFv, di-scFv, tri-scFv, and other single chain variable fragments, and other cell-penetrating molecules disclosed herein are also expressly provided for use in compositions, conjugates, and methods disclosed herein. Autoantibodies against double-stranded deoxyribonucleic acid (dsDNA) are frequently identified in the serum of patients with systemic lupus erythematosus (SLE) and are often implicated in disease pathogenesis. Therefore, in embodiments, cell-penetrating antibodies (e.g., cell-penetrating anti-DNA antibodies) can be derived or isolated from patients with SLE or animal models of SLE.

As used herein, “antibody drug conjugate” or “ADC” refers to an antibody or antigen-binding fragment or variant thereof that is covalently linked or conjugated to a biologically active molecule, for example, a drug or an anti-tumor drug.

As used herein, a “linker” is any chemical moiety that is capable of linking or connecting a compound, usually a drug or an anti-tumor drug, to a cell-binding agent such as an antibody, such as a 3E10 antibody or a fragment thereof, in a stable, covalent manner. In embodiments, a “linker” is any chemical moiety that is capable of linking or connecting a compound such as a DNA damage-inducing agent, a DNA repair inhibitor, an immune modulatory molecule, an alkylating agent, a microtubule inhibitor, an immune checkpoint inhibitor, an angiogenesis inhibitor, an adoptive cell therapy, or a topoisomerase inhibitor, to a cell-binding agent such as a 3E10 antibody or a fragment thereof, in a stable, covalent manner. Linkers can be susceptible to or be substantially resistant to acid-induced cleavage, light-induced cleavage, peptidase-induced cleavage, esterase-induced cleavage, and/or disulfide bond cleavage, at conditions under which the compound and/or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art.

As used herein, the term “subject” refers to any individual who is the target of administration. The subject can be any animal (e.g., a mammal), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment). In embodiments, the subject is a human.

In embodiments, methods of the disclosure are useful in treatment a human subject. In embodiments, the human may be referred to as a patient. In embodiments, the human is a female. In embodiments, the human is a male. In embodiments, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

The terms “cancer” and “cancerous” refer to or describe the physiological condition in mammals in which a population of cells are characterized by unregulated cell growth. Examples of cancer include, but are not limited to, colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumors.

“Tumor” and “neoplasm” refer to any mass of tissue that results from excessive cell growth or proliferation, either benign (noncancerous) or malignant (cancerous), including pre-cancerous lesions.

The terms “cancer cell,” “tumor cell,” and grammatical equivalents thereof refer to the total population of cells derived from a tumor or a pre-cancerous lesion, including both non-tumorigenic cells, which comprise the bulk of the tumor cell population, and tumorigenic stem cells (cancer stem cells).

The term “chemotherapeutic warhead” refers to a cytotoxic drug or anti-tumor drug covalently linked to an antibody in an ADC.

As used herein, the term “pharmaceutically effective amount” means that the amount of the composition used is of sufficient quantity to ameliorate one or more causes or symptoms of a disease or disorder. Such amelioration only requires a reduction or alteration, not necessarily elimination. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “carrier” or “excipient” refers to an organic or inorganic ingredient, natural or synthetic inactive ingredient in a formulation, with which one or more active ingredients are combined. In embodiments, a carrier or excipient is selected to minimize degradation of the active ingredient or to minimize adverse side effects in the subject, as would be well known to one of skill in the art.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A “pharmacologically effective amount,” “pharmacologically effective dose,” “therapeutically effective amount,” or “effective amount” refers to an amount sufficient to produce the desired physiological effect or amount capable of achieving the desired result, particularly for treating or preventing the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures, tissue samples, tissue homogenates or experimental animals, e.g., for determining the LD50 (the dose lethal to about 50% of the population) and the ED50 (the dose therapeutically effective in about 50% of the population) or the maximum tolerated dose. The dosage can vary depending upon the dosage form employed and the route of administration utilized. The dose ratio between toxic and therapeutic effects is the therapeutic index and can be expressed as the ratio LD50/ED50. In embodiments, compositions, conjugates, and methods that exhibit large therapeutic indices are preferred. A therapeutically effective dose can be estimated initially from in vitro assays, including, for example, cell culture assays or measurements. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In embodiments, the effect will result in a quantifiable change of at least about 10%, at least about 20%, at least about 30%, at least about 50%, at least about 70%, or at least about 90%. In embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

### III. Antibody Drug Conjugates (ADCs)

The present disclosure is directed, in part, to antibody drug conjugates (ADCs) comprising a cell-penetrating antibody, e.g., a 3E10 antibody or a or antigen-binding fragment or variant thereof, conjugated via a linker to a biologically active molecule or a therapeutic agent.

In embodiments, an ADC specifically binds its epitope, and triggers cell death via delivery of the drug or anti-tumor drug. In embodiments, an antibody-drug conjugate (ADC) has the formula A-(L-Pᵣ)_{q}, wherein: A is a 3E10 antibody or antigen-binding fragment or variant thereof, L is a linker, and P is a payload as described herein.

In embodiments, the present disclosure relates to the use of 3E10 antibodies, and derivatives thereof, for delivering therapeutic agents to a subject. Although generally referred to herein as “3E10” or “3E10 antibodies,” it will be appreciated that fragments, variants, and binding proteins, including antigen-binding fragments and fusion proteins, such as scFv, di-scFv, tr-scFv, and other single chain variable fragments, and other cell-penetrating, nucleic acid transporting molecules disclosed herein, are encompassed by the phrase and are also expressly provided for use in compositions, conjugates, and methods disclosed herein. Thus, the antibodies and other binding proteins are also referred to herein as cell-penetrating.

In embodiments, the antibody is conjugated to the therapeutic agent via a linker. In embodiments, the antibody is a 3E10 antibody or antigen-binding fragment or variant thereof, as described herein. In embodiments, the antibody is a humanized 3E10 antibody or antigen-binding fragment or variant thereof, as described herein. Any variety of agents can be transported via conjugation to the 3E10 antibody or antigen-binding fragment or variant thereof, or humanized 3E10 antibody or antigen-binding fragment or variant thereof, herein, such as inorganic and organic molecules, pharmaceutical agents, drugs, peptides, proteins, genetic material, and the like. In embodiments, the antibody-drug conjugate (ADC) comprises a drug, or an anti-tumor drug.

### A. Antigen-Binding Domains (ABDs)

As used herein, the term “antigen-binding domain” or “ABD” refers to a domain comprising a three-dimensional structure capable of immunospecifically binding to an epitope of an antigen. Thus, in embodiments, an ABD comprises a hypervariable region, optionally a VH and/or VL domain of an antibody, optionally at least a VH domain. In embodiments, an ABD comprises at least one complementarity determining region (CDR) of an antibody. In embodiments, an ABD comprises at least two CDRs of an antibody. In embodiments, an ABD comprises at least three CDRs of an antibody. In embodiments, an ABD comprises at least four CDRs of an antibody. In embodiments, an ABD comprises at least five CDRs of an antibody. In embodiments, an ABD comprises six CDRs of an antibody.

### 1. 3E10 Antibodies

In embodiments, the present disclosure relates to the use of 3E10 antibodies, and derivatives thereof, for delivering therapeutic agents to a subject. Although generally referred to herein as “3E10” or “3E10 antibodies,” it will be appreciated that fragments, variants, and binding proteins, including antigen-binding fragments and fusion proteins, such as scFv, di-scFv, tr-scFv, and other single chain variable fragments, and other cell-penetrating, nucleic acid transporting molecules disclosed herein, are encompassed by the phrase and are also expressly provided for use in compositions, conjugates, and methods disclosed herein. Thus, the antibodies and other binding proteins are also referred to herein as cell-penetrating.

In embodiments, a 3E10 antibody comprises VL CDRs of SEQ ID NOs: 61, 62, and 63 and VH CDRs of SEQ ID NOs: 58, 59, and 60. In embodiments, a 3E10 antibody comprises VL CDRs of SEQ ID NOs: 9, 10, and 11 and VH CDRs of SEQ ID NOs: 3, 4, and 5. In embodiments, a 3E10 antibody comprises VL CDRs of SEQ ID NOs: 22, 23, and 24 and VH CDRs of SEQ ID NOs: 15, 17, and 18. In embodiments, a 3E10 antibody comprises VL CDRs of SEQ ID NOs: 9, 10, and 11 and VH CDRs of SEQ ID NOs: 16, 4, and 5.

In embodiments, a 3E10 antibody has nucleic acid binding affinity.

In embodiments, a 3E10 antibody is competent for ENT2-mediated cell internalization when bound to nucleic acid.

In embodiments, targeting of an ADC described herein is not limited to a tissue- or cancer-specific antigen or tumor epitope when used in a method of treating cancer, or when targeting a cancer cell or tumor or tumor cell. Accordingly, an ADC provided herein can widely target cancers based on cell surface expression of ENT2, a nucleoside transporter overexpressed in cancer cells and tumor cells. Advantageously, an ADC described herein can target ENT2 and extracellular DNA simultaneously. Importantly, 3E10 has been shown to preferentially localize into tumor cell nuclei in vivo, likely due to increased DNA in the local environment released from ischemic and necrotic regions of tumor. Targeting of the 3E10 antibody to extracellular DNA is described in, for example in Weisbart, Sci Reports, 2015, which is herein incorporated by reference. By targeting ENT2 as well as extracellular DNA, the 3E10 ADC described herein presents a platform to target a variety of cancers and deliver chemotherapeutic drugs to target and kill cancer cells.

In embodiments of the present disclosure, the antibody or antigen-binding fragment or variant thereof is murine, chimeric, humanized, or human.

In embodiments, an ADC of the present disclosure penetrates into cells and nuclei in an ENT2-dependent manner.

In embodiments, an ADC of the present disclosure further comprises a bound polynucleotide, wherein A binds the polynucleotide. In embodiments, the bound polynucleotide is precomplexed with the ADC. In embodiments, the bound polynucleotide is an extracellular polynucleotide that is bound by the ADC at a site of interest, such as, for example, at a site of tumor ischemia and necrosis. In embodiments, the polynucleotide is DNA. In embodiments, the polynucleotide is RNA.

In embodiments, an ABD comprises a VH and/or VL domain of a 3E10 antibody. In embodiments, the ABD comprises a VH CDR1, VH CDR2, VH CDR3, VL CDR1, VL CDR2, and/or VL CDR3 of a 3E10 antibody.

In embodiments, the present disclosure provides an antibody-drug conjugate (ADC) having the formula A-(L-Pᵣ)_{q}, wherein: A is a 3E10 antibody or antigen-binding fragment or variant thereof, L is a linker, and P is a payload as described herein, wherein the linker L links A to P. In embodiments of the present disclosure, the amino acid residue corresponding with D31 of the heavy chain CDR1 of the 3E10 antibody or antigen-binding fragment or variant thereof is substituted with N. It is known in the art that mutation of aspartic acid at residue 31 of VH CDR1 to asparagine increases the cationic charge of this residue and enhanced nucleic acid binding and delivery in vivo (3E10-D31N). In embodiments, additional 3E10 antibody variants include mutation of aspartic acid at residue 31 of VH CDR1 to arginine (3E10-D31R), which modeling indicates expands cationic charge, or lysine (3E10-D31K) which modeling indicates changes charge orientation. Thus, in embodiments, the 3E10 antibody or antigen-binding fragment or variant thereof includes a D31R or D31K substitution. In embodiments, additional 3E10 antibody variants include mutation a R96N, and/or S30D substitution, alone or in combination with D31N, D31R, or D31K. All of the sequences disclosed herein having the residue corresponding to 3E10 D31 or N31, are expressly disclosed with a D31R or D31K or N31R or N31K substitution.

In embodiments, the present disclosure provides an antibody-drug conjugate (ADC) having the formula A-(L-Pᵣ)_{q}, wherein: A is a 3E10 antibody or antigen-binding fragment or variant thereof, L is a linker, P is a payload as described herein, r is an integer from 1 to 4, and q is an integer from 1 to 16, wherein the linker L links A to (P); wherein the 3E10 antibody or antigen-binding fragment thereof comprises a heavy chain variable region (VH) CDR1 comprising the amino acid sequence of X1YGMX2, where X1 is D, E, N, Q, R, or K and X2 is K, R, or H (SEQ ID NO:58). In embodiments, the antibody or antigen-binding fragment or variant thereof comprises (a) a light chain variable region (VL) complementarity determining region (CDR) 1 comprising the amino acid sequence of X1ASX2X3VSTSSYSYX4X5, where X1 is K, R, or H, X2 is K, R, or H, X3 is T or S, X4 is M or L, and X5 is K, R, H, or A (SEQ ID NO:61), (b) a VL CDR2 comprising the amino acid sequence of YASYLX1S, where X1 is D, E, N, or Q (SEQ ID NO:62), and (c) a VL CDR3 comprising the amino acid sequence of QX1SX2X3FPWT, where X1 is K, R, or H, X2 is K, R, or H, and X3 is D or E (SEQ ID NO:63), and (d) a heavy chain variable region (VH) CDR1 comprising the amino acid sequence of X1YGMX2, where X1 is D, E, N, Q, R, or K and X2 is K, R, or H (SEQ ID NO:58), (e) a VH CDR2 comprising the amino acid sequence of YISSX1SSTIYYAX2X3VX4G, where X1 is G or S, X2 is D or E, X3 is T or S, and X4 is K, R, or H (SEQ ID NO:59), and (f) a VH CDR3 comprising the amino acid sequence of X1GLLLX2Y, where X1 is K, R, or H, and X2 is D or E (SEQ ID NO:60).

In embodiments, the present disclosure provides an antibody-drug conjugate (ADC) having the formula A-(L-Pr)q, wherein: A is a 3E10 antibody or antigen-binding fragment or variant thereof, L is a linker, P is a payload as described herein, r is an integer from 1 to 4, and q is an integer from 1 to 16, wherein the linker L links A to P; wherein the 3E10 antibody or antigen-binding fragment thereof comprises a heavy chain variable region (VH) CDR1 comprising the amino acid sequence of NYGMH (SEQ ID NO: 15). In embodiments, the antibody or antigen-binding fragment or variant thereof comprises (a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO:10), and (c) a VL CDR3 comprising the amino acid sequence of QHSREFPWT (SEQ ID NO: 11), and (d) a heavy chain variable region (VH) CDR1 comprising the amino acid sequence of NYGMH (SEQ ID NO: 15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5). In embodiments, the antibody or antigen-binding fragment or variant thereof comprises a light chain variable region (VL) comprising an amino acid sequence that is identical to SEQ ID NO:21. In embodiments, the antibody or antigen-binding fragment or variant thereof comprises a heavy chain variable region (VH) comprising an amino acid sequence that is identical to SEQ ID NO:14. In embodiments, the antibody or antigen-binding fragment or variant thereof comprises a full length light chain (LC) comprising an amino acid sequence that is identical to SEQ ID NO:20. In embodiments, the antibody or antigen-binding fragment or variant thereof comprises a full length heavy chain (HC) comprising an amino acid sequence that is identical to SEQ ID NO:13.

In embodiments, the antibody or antigen-binding fragment or variant thereof comprises a light chain variable region (VL) comprising an amino acid sequence that is at least about 80%, at least about 85%, at least about 90%, at least about 95%, at least about 96%, at least about 97%, at least about 98%, or at least about 99% identical to SEQ ID NO:21. In embodiments, the antibody or antigen-binding fragment or variant thereof comprises a heavy chain variable region (VH) comprising an amino acid sequence that is at least about 80%, at least about 85%, at least about 90%, at least about 95%, at least about 96%, at least about 97%, at least about 98%, or at least about 99% identical to SEQ ID NO:14. In embodiments, the antibody or antigen-binding fragment or variant thereof comprises a full length light chain (LC) comprising an amino acid sequence that is at least about 80%, at least about 85%, at least about 90%, at least about 95%, at least about 96%, at least about 97%, at least about 98%, or at least about 99% identical to SEQ ID NO:20. In embodiments, the antibody or antigen-binding fragment or variant thereof comprises a full length heavy chain (HC) comprising an amino acid sequence that is at least about 80%, at least about 85%, at least about 90%, at least about 95%, at least about 96%, at least about 97%, at least about 98%, or at least about 99% identical to SEQ ID NO:13.

In embodiments, the 3E10 antibody or antigen-binding fragment or variant thereof can be transported into the cytoplasm and/or nucleus of the cells without the aid of a carrier or conjugate. For example, monoclonal antibody 3E10 and active fragments thereof that are transported in vivo to the nucleus of mammalian cells without cytotoxic effect are disclosed in U.S. Pat. Nos. 4,812,397 and 7,189,396 to Richard Weisbart (each of which is incorporated by reference herein, in its entirety).

Amino acid sequences of 3E10 monoclonal antibodies and antigen-binding fragments thereof are known in the art. Example sequences of 3E10 heavy and light chains are provided below herein.

The murine version of the 3E10 antibody is described in Zack, et al., Immunology and Cell Biology, 72:513-520 (1994) (which is incorporated by reference herein, in its entirety).

Amino acid variants of the 3E10 antibody are also known in the art, for example, as described in Zack, et al., J. Immunol., 157(5):2082-8 (1996). For example, amino acid position 31, in CDR1 of the heavy chain variable region of 3E10, influences nucleic acid binding and the antibody's ability to penetrate nuclei. Substitution of the ‘wild-type’ (e.g., relative to the original murine antibody) aspartic acid by asparagine (the ‘D31N’ mutation) improves nucleic acid binding and nuclei penetration of the antibody, relative to the ‘wild type’ murine antibody. See, for example, Zack, et al., Immunology and Cell Biology, 72:513-520 (1994); Weisbart, et al., J. Autoimmun., 11, 539-546 (1998); and Weisbart, Int. J. Oncol., 25, 1867-1873 (2004) (which are incorporated by reference herein, in their entireties).

Sequences for 3E10 antibodies and antigen-binding fragments or variants thereof, with the D31N substitution, are disclosed herein. In embodiments, the 3E10 antibodies and binding fragments or variants thereof disclosed herein include the D31N substitution. In embodiments, other amino acids are substituted at position 31 in the 3E10 antibodies and antigen-binding fragments or variants thereof disclosed herein. For example, D31R, D31K, or D31R substitutions are incorporated in embodiments of the present disclosure.

Other 3E10 light chain sequences are known in the art. See, for example, Zack, et al., J. Immunol., 15; 154(4):1987-94 (1995); GenBank: L16981-1—Mouse Ig rearranged L-chain gene, partial cds; GenBank: AAA65681-1—immunoglobulin light chain, partial [Mus musculus]).

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. In embodiments, antibodies disclosed herein are IgA, IgD, IgE, Ig1, or IgM, including any subtype or isotype thereof. In embodiments, antibodies disclosed herein are based on the IgG class. In embodiments, antibodies disclosed herein are based on one of the subclasses of IgG, including, but not limited to IgG1, Ig12, Ig13, and IgG4. In general, Ig11, Ig12 and IgG4 are used more frequently than IgG3. It should be noted that IgG1 has different allotypes with polymorphisms at 356 (D or E) and 358 (L or M), and in embodiments, antibodies disclosed herein are based on IgG1 having D or E at position 356 and/or L or M at position 358.

The light chain generally comprises two domains, the variable light domain (containing the light chain CDRs and together with the variable heavy domains forming the Fv region), and a constant light chain region (often referred to as CL or CK). The heavy chain comprises a variable heavy domain and a constant domain, which includes a CHI-optional hinge-Fc domain comprising a CH2-CH3.

The hypervariable region of an antibody generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST·5th Ed. Public Health Service, National Institutes of Health, Bethesda, Md. (1991) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs useful for the compositions, conjugates, and methods described herein are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the VH CDRs (e.g., VH CDR1, VH CDR2 and VH CDR3) and the disclosure of each variable light region is a disclosure of the VL CDRs (e.g., VL CDR1, VL CDR2 and VL CDR3).

Throughout the present disclosure, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)). In embodiments, the present specification uses the the IMGT system to define the complementarity determining regions (CDRs) provided herein.

The present disclosure provides a large number of different CDR sets. In this case, a “full CDR set” comprises the three variable light CDRs, e.g., a VL CDR1, VL CDR2, and VL CDR3, and the three variable heavy CDRs, e.g., VH CDR1, VH CDR2, and VH CDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As noted above herein, the present disclosure refers to different antibody domains of a 3E10 antibody or antigen-binding fragment or variant thereof. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy (VH) domain, the variable light (VL) domain, the light constant domain, Fab domains and scFv domains.

### 2. Humanized Antibodies

In embodiments, antibodies of the disclosure comprise a heavy chain variable region from a particular germline heavy chain immunoglobulin gene and/or a light chain variable region from a particular germline light chain immunoglobulin gene. For example, such antibodies can comprise or consist of murine, chimeric, humanized, or human antibodies or antigen-binding fragments or variants thereof comprising heavy or light chain variable regions that are “the product of or “derived from” a particular germline sequence, e.g., that of the 3E10 antibody. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of or “derived from” a particular human germline immunoglobulin sequence can contain amino acid differences as compared to the germline sequence, due to, for example, naturally-occurring somatic mutations or intentional introduction of site-directed mutation. However, a humanized antibody typically is at least about 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody can be at least about 95%, at least about 96%, at least about 97%, at least about 98%, or at least about or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody can display no more than 5, or even no more than 4, 3, 2, or 1 amino acid difference from the amino acid sequence encoded by the germline immunoglobulin gene.

In one aspect, the parent antibody has been affinity matured, as is known in the art. Structure-based methods can be employed for humanization and affinity maturation, for example as described in U.S. Ser. No. 11/004,590, which is incorporated herein by reference. Selection based methods can be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all of which are incorporated herein by reference. Other humanization methods can involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all of which are incorporated herein by reference.

### 3. Fc Variants

In embodiments, one or more amino acid modifications can be introduced into the Fc region of an antibody provided herein, thereby generating an Fc region variant. The Fc region variant can comprise a human Fc region sequence (e.g., a human IgG1, Ig12, Ig13 or IgG4 Fc region) comprising an amino acid modification (e.g. a substitution) at one or more amino acid positions.

In embodiments, an Fc region variant possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays known in the art can be conducted to ensure that the antibody lacks FcγR binding (hence likely lacking ADCC activity), but retains FcRn binding ability. To assess complement activation, a CDC assay can be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

In embodiments, an antibody provided herein can have reduced effector function and thus can comprise a substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In embodiments, an Fc region variant provided herein can have improved or diminished binding to FcRs. (See, e.g., U.S. Pat. No. 6,737,056; WO 2004/056312, and Shields et al., J. Biol. Chem. 9(2): 6591-6604 (2001).)

In embodiments, an Fc region variant provided herein comprises an Fc region with one or more amino acid substitutions which improve ADCC, e.g., substitutions at positions 298, 333, and/or 334 of the Fc region (EU numbering of residues).

In embodiments, an Fc region variant provided herein comprises alterations that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In embodiments, an Fc region variant provided herein comprises alterations that result in increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), e.g., as described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

In embodiments, an Fc region variant provided herein comprises “knob-in-hole” or “skew” variants, which reder to amino acid engineering that creates stearic influences to favor heterodimeric formation and disfavor homodimeric formation, as described in U.S. Ser. No. 61/596,846, Ridgway et al, Protein Engineering 9(7):617 (1996); Atwell et al, J. Mol. Biol. 1997 270:26; U.S. Pat. No. 8,216,805, all of which are hereby incorporated by reference in their entirety.

In embodiments, an Fc region variant provided herein comprises alterations described in Duncan & Winter, Nature 322:738-40 (1988); U.S. Pat. Nos. 5,648,260; 5,624,821; and WO 94/29351.

### 4. Antibody Fragments

In embodiments, the antibody portion of an ADC described herein comprises an antigen-binding fragment of a 3E10 antibody or variant thereof. In embodiments, the antigen-binding fragment retains the desired biological activity of a 3E10 antibody. In embodiments, the antigen-binding fragment retains at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% of the desired biological activity of a 3E10 antibody. In embodiments, the antigen-binding fragment retains the ability of the antibody to bind to its target antigen, e.g., a nucleic acid, e.g., DNA. In embodiments, the antigen-binding fragment retains the ability of the antibody to bind to a cellular receptor, e.g., ENT2. In embodiments, the antigen-binding fragment retains the ability of the antibody to be internalized by a target cell.

In embodiments, the 3E10 antibody or antigen binding fragment thereof comprises a single-chain fragment variable (scFv), a tandem double scFv, an (scFv)2, a minibody, a VHH, an scFv-Fc, a CrossMab, a dual variable domain immunoglobulin (DVD-Ig), a single-chain tandem fragment variable (scTaFv), a diabody, a tandem diabody (TandAb), a Fabsc, a modular IgG-scFv, a Fab, or an F(ab′)2.

In embodiments, an antigen-binding fragment of a 3E10 antibody or variant thereof comprises a CrossMab. In the CrossMab format, complementary mutations are introduced in the heavy chain constant region of each arm to generate so-called “holes and knobs,” resulting in preferred association between different arms, forming a heterodimer, rather than a homodimer of two of the same arms. The exact residues that are mutated in the heavy chain constant region of a CrossMab bispecific antibody to form “holes” and “knobs” can vary depending on the specific design and optimization goals of the antibody. For more information on CrossMab antibodies see, for example, Huang, J., et al., Journal of Biological Chemistry, 294(50):19001-10 (2019), the disclosure of which is incorporated herein by reference in its entirety.

In embodiments, an antigen-binding fragment of a 3E10 antibody or variant thereof comprises a divalent, dual-variable domain immunoglobulin (DVD-Ig) of a 3E10 antibody or antigen binding fragment thereof. In the DVD-Ig format, each arm of the antibody contains two VH/VL pairs. In some embodiments, one of the VH/VL pairs comprises 3E10 VH and VL CDRs. For more information on DVD-Ig antibodies see, for example, Polson A G, et al., Journal of immunotherapy. 29(3):241-50 (2006) and U.S. Pat. No. 7,612,181, the disclosures of which are incorporated herein by reference in their entireties.

In embodiments, an antigen-binding fragment of a 3E10 antibody or variant thereof comprises a single-chain variable fragment (scFv). “Single chain Fv” or “scFv,” as used herein, refers to a VH domain covalently attached to a VL domain through a linker, e.g., a scFv linker as discussed herein, to form a continuous protein chain. A scFv domain can be in either arrangement from N- to C-terminus (i.e., VH-linker-VL or VL-linker-VH). In the sequences depicted in the sequence listing and in the figures herein, the order of the VH and VL domain is indicated in the name, e.g., H.X_L.Y means the N- to C-terminus arrangement is VH-linker-VL, and L.Y_H.X means the N- to C-terminus arrangement is VL-linker-VH.

In embodiments, an antigen-binding fragment of a 3E10 antibody or variant thereof comprises a tandem double scFv. A tandem double scFv has two scFv domains linked in a linear fashion. In some embodiments, each scFv domain is derived from a different antibody and provides independent antigen-binding specificity. In some embodiments, one of the scFv domains comprises 3E10 VH and VL CDRs. For more information on tandem double scFvs see, for example, Bossen C, et al., MAbs, 4(2):200-08 (2012), the disclosure of which is incorporated herein by reference in its entirety.

In embodiments, an antigen-binding fragment of a 3E10 antibody or variant thereof comprises a dimeric scFv antibody (scFv)2. A dimeric scFv antibody has two scFv domains linked in a dimeric arrangement. In some embodiments, each scFv domain is derived from a different antibody and provides independent antigen-binding specificity. In some embodiments, one of the scFv domains comprises 3E10 VH and VL CDRs. For more information on dimeric scFv antibodies see, for example, Llewellyn C, et al., Journal of immunological methods, 273(1-2):33-44 (2002), the disclosure of which is incorporated herein by reference in its entirety.

In embodiments, an antigen-binding fragment of a 3E10 antibody or variant thereof comprises a scFv-Fc. An “scFv-Fc,” as meant herein, is a polypeptide that consists of a heavy and a light chain variable region of an antibody joined by a linker, which is followed by an Fc polypeptide chain of an antibody, optionally the Fc region of a human IgG antibody, such as an IgG1, Ig12, Ig13, or IgG4 antibody.

In embodiments, an antigen-binding fragment of a 3E10 antibody or variant thereof comprises a single-chain tandem fragment variable (scTaFv) antibody. A single-chain tandem fragment variable (scTaFv) antibody is a type of bispecific antibody that consists of two variable fragment (VH and VL) domains linked in a tandem arrangement. In some embodiments, one of the variable fragment domains comprises 3E10 VH and VL CDRs. For more information on scTaFv antibodies see, for example, Schramm C, et al., MAbs 5(3):442-49 (2013), the disclosure of which is incorporated herein by reference in its entirety.

In embodiments, an antigen-binding fragment of a 3E10 antibody or variant thereof comprises a VHH. As used herein, the term “VHH” refers to a variable domain of heavy chain of heavy-chain antibody. A VHH is a molecule that can recognize an antigen through a single domain and is the smallest unit among antibody molecules that have been found to date. In embodiments, a VHH can include one or more variable domains of heavy chain derived from a heavy-chain antibody, and the number of the variable domains of heavy chain included in the VHH is not limited.

In embodiments, an antigen-binding fragment of a 3E10 antibody or variant thereof comprises a diabody. As used herein, “diabody” refers to a divalent antibody comprising two polypeptide chains, wherein each polypeptide chain is too short for a pair to form between two domains on the same chain such that each domain is paired with a complementary domain on another polypeptide chain (see, e.g., Holliger et al., 1993, Proc. Natl. Acad. Sci. USA 90: 6444-48 and Poljak et al., 1994, Structure 2: 1121-23). If the two polypeptide chains of the diabody are identical, there will be two identical antigen-binding sites in the diabody resulting from their pairing. In embodiments, one of the antigen binding domains of the diabody comprises 3E10 VH and VL CDRs. For more information on diabodies see, for example, Hoogenboom H R, et al., Trends Biotechnol., 21(12):553-57 (2003), the disclosure of which is incorporated herein by reference in its entirety. Polypeptide chains of different sequences can be used to prepare diabodies with two different antigen-binding sites. Similarly, as used herein, “triabodies” and “tetrabodies” refer to antibodies that contain three and four polypeptide chains, respectively, and form three and four antigen-binding sites (which can be the same or different), respectively.

The term “minibody” is used to refer to an scFv-CH₃ fusion protein that self- assembles into a bivalent dimer of 80 kDa (ScFv-CH₃)2.

In embodiments, an antigen-binding fragment of a 3E10 antibody or variant thereof comprises a tandem diabody (TandAb). A tandem diabody has wo antigen-binding domains (VH and VL) linked in a tandem arrangement by a flexible peptide linker. In some embodiments, one of the antigen binding domains comprises 3E10 VH and VL CDRs. For more information on diabodies see, for example, Sidelmann J G, et al., Mol Immunol., 45(9):2597-607 (2008), the disclosure of which is incorporated herein by reference in its entirety.

In embodiments, an antigen-binding fragment of a 3E10 antibody or variant thereof comprises a Fabsc. As used herein, a “Fabsc” format antibody molecule typically refers to a bispecific antibody molecule having a Fab fragment, which generally includes a hinge region, which is at the C-terminus of the Fab fragment linked to the N- terminus of a CH₂ domain, of which the C-terminus is in turn linked to the N-terminus of a scFv fragment.

In embodiments, an antigen-binding fragment of a 3E10 antibody or variant thereof comprises a scFab. A scFab, also known as a single-chain fragment antigen binding (Fab), is a type of antibody fragment that combines the variable heavy chain (VH) and variable light chain (VL) domains into a single polypeptide chain, linked by a peptide linker. The domain structure of a Fabsc includes the variable domains of both the heavy chain and light chain (VH and VL), and a peptide linker that connects the two domains. In addition to the variable domains, a Fabsc also includes the constant domains of the light chain (CL) and the hinge region of the heavy chain. In some embodiments, one of the antigen binding domains comprises 3E10 VH and VL CDRs. For more information on Fabscs see, for example, Kettner, C., et al., Frontiers in Immunology, 8(8):453 (2017), the disclosure of which is incorporated herein by reference in its entirety.

In embodiments, an antigen-binding fragment of a 3E10 antibody or variant thereof comprises an IgG-scFv. An IgG-scFv is an antibody in which a scFv is fused to the light chain or heavy chain of an IgG. In some embodiments, the scFv comprises 3E10 VH and VL CDRs. In some embodiments, the IgG comprises 3E10 VH and V LCDRs. In some embodiments, the antibody is an F(ab′)2.

### 5. Bispecific Antibodies

In embodiments, 3E10 antibodies and antigen-binding fragments or variants thereof can be modified to improve their therapeutic potential. For example, in embodiments, the cell-penetrating anti-DNA antibody is conjugated to another antibody specific for a second therapeutic target in the cytoplasm and/or nucleus of a target cell. For example, in embodiments, the cell-penetrating 3E10 antibody is a bispecific antibody having a first heavy chain and a first light chain from 3E10 and a second heavy chain and a second light chain from a monoclonal antibody that specifically binds a second therapeutic target.

Bispecific antibodies and other binding proteins having a first heavy chain and a first light chain from 3E10 and a second heavy chain and a second light chain from a monoclonal antibody that specifically binds a second target are discussed in Weisbart, et al., Mol. Cancer Ther., 11(10):2169-73 (2012), and Weisbart, et al., Int. J. Oncology, 25:1113-8 (2004), and U.S. Patent Application No. 2013/0266570, which are specifically incorporated by reference in their entireties. In embodiments, the second target is specific for a target cell-type, tissue, organ, etc. Thus the second heavy chain and second light chain can serve as a targeting moiety that targets the complex to the target cell-type, tissue, organ. In embodiments, the second heavy chain and second light chain target, hematopoietic stem cells, CD34⁺ cells, T cells or any another cell type of interest, e.g., by targeting a receptor or ligand expressed on the cell type of interest. In embodiments, the second heavy chain and second light chain target the thymus, spleen, or cancer cells.

Bispecific antibodies can be used to direct cytotoxic agents or drugs to cells which express a particular antigen. These antibodies possess two binding sites directed at two different antigens or two different epitopes on the same antigen. For example, in embodiments the bispecific can comprise one arm for ENT2 engagement and another arm for a second target. Bispecific antibody design can include a variety of antibody designs with multiple binding arms. Techniques for making bispecific antibodies are common in the art (Millstein et al., 1983, Nature 305:537-539; Brennan et al., 1985, Science 229:81; Suresh et al, 1986, Methods in Enzymol. 121:120; Traunecker et al., 1991, EMBO J. 10:3655-3659; Shalaby et al., 1992, J. Exp. Med. 175:217-225; Kostelny et al., 1992, J. Immunol. 148:1547-1553; Gruber et al., 1994, J. Immunol. 152:5368; and U.S. Pat. No. 5,731,168). Antibodies with more than two valencies are also contemplated. For example, trispecific antibodies can be prepared (Tutt et al., J. Immunol. 147:60 (1991)). In embodiments the contemplated bispecific antibody disclosed herein can be conjugated as a bispecific ADC.

Heteroconjugate antibodies are also within the scope of the present disclosure. Heteroconjugate antibodies are composed of two covalently joined antibodies. Such antibodies have, for example, been proposed to target immune cells to unwanted cells (U.S. Pat. No. 4,676,980). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate. In embodiments the contemplated herteoconjugate antibody disclosed herein can be conjugated as a heteroconjugate ADC.

### 6. 3E10 Sequences

In embodiments, a 3E10 antibody or variant thereof, or antigen-binding fragment thereof described herein includes CDR sequences corresponding to the parent 3E10 antibody.

Accordingly, in embodiments, a 3E10 antibody or variant thereof, or antigen-binding fragment thereof comprises (a) a light chain variable region (VL) complementarity determining region (CDR) 1 comprising the amino acid sequence of X1ASX2X3VSTSSYSYX4X5, where X1 is K, R, or H, X2 is K, R, or H, X3 is T or S, X4 is M or L, and X5 is K, R, H, or A (SEQ ID NO:61), (b) a VL CDR2 comprising the amino acid sequence of YASYLX1S, where X1 is D, E, N, or Q (SEQ ID NO:62), and (c) a VL CDR3 comprising the amino acid sequence of QX1SX2X3FPWT, where X1 is K, R, or H, X2 is K, R, or H, and X3 is D or E (SEQ ID NO:63), and (d) a heavy chain variable region (VH) CDR1 comprising the amino acid sequence of X1YGMX2, where X1 is D, E, N, Q, R, or K and X2 is K, R, or H (SEQ ID NO:58), (e) a VH CDR2 comprising the amino acid sequence of YISSX1SSTIYYAX2X3VX4G, where X1 is G or S, X2 is D or E, X3 is T or S, and X4 is K, R, or H (SEQ ID NO:59), and (f) a VH CDR3 comprising the amino acid sequence of X1GLLLX2Y, where X1 is K, R, or H, and X2 is D or E (SEQ ID NO:60).

In embodiments, a 3E10 antibody or variant thereof, or antigen-binding fragment thereof includes a light chain variable region (VL) complementarity determining region (CDR) 1 comprising the amino acid sequence of 3E10-VL-CDR1 (SEQ ID NO:9), a VL CDR2 comprising the amino acid sequence of 3E10-VL-CDR2 (SEQ ID NO:10), a VL CDR3 comprising the amino acid sequence of 3E10-VL-CDR3 (SEQ ID NO:11), a heavy chain variable region (VH) CDR1 comprising the amino acid sequence of 3E10-VH-CDR1 (SEQ ID NO:3), a VH CDR2 comprising the amino acid sequence of 3E10-VH-CDR2 (SEQ ID NO:4), and a VH CDR3 comprising the amino acid sequence of 3E10-VH-CDR3 (SEQ ID NO:5).

In embodiments, a 3E10 antibody or variant thereof, or antigen-binding fragment thereof described herein includes CDR sequences from a variant 3E10 antibody that includes a D31N amino acid substitution in the VH CDR1. Accordingly, in embodiments, the a 3E10 antibody or variant thereof, or antigen-binding fragment thereof includes a light chain variable region (VL) complementarity determining region (CDR) 1 comprising the amino acid sequence of 3E10-VL-CDR1_D31N (SEQ ID NO:22), a VL CDR2 comprising the amino acid sequence of 3E10-VL-CDR2_D31N (SEQ ID NO:23), a VL CDR3 comprising the amino acid sequence of 3E10-VL-CDR3_D31N (SEQ ID NO:24), a heavy chain variable region (VH) CDR1 comprising the amino acid sequence of 3E10-VH-CDR1 D31N (SEQ ID NO:15), a VH CDR2 comprising the amino acid sequence of 3E10-VH-CDR2_D31N (SEQ ID NO:17), and a VH CDR3 comprising the amino acid sequence of 3E10-VH-CDR3_D31N (SEQ ID NO:18).

In embodiments, a 3E10 antibody or variant thereof, or antigen-binding fragment thereof described herein refers to CDR sequences corresponding to the parent 3E10 antibody, optionally including a D31N amino acid substitution in the VH CDR1. Accordingly, in embodiments, a 3E10 antibody or variant thereof, or antigen-binding fragment thereof includes a light chain variable region (VL) complementarity determining region (CDR) 1 comprising the amino acid sequence of 3E10-VL-CDR1 (SEQ ID NO:9), a VL CDR2 comprising the amino acid sequence of 3E10-VL-CDR2 (SEQ ID NO:10), a VL CDR3 comprising the amino acid sequence of 3E10-VL-CDR3 (SEQ ID NO:11), a heavy chain variable region (VH) CDR1 comprising the amino acid sequence of 3E10-VH-CDR1a (SEQ ID NO:16), a VH CDR2 comprising the amino acid sequence of 3E10-VH-CDR2 (SEQ ID NO:4), and a VH CDR3 comprising the amino acid sequence of 3E10-VH-CDR3 (SEQ ID NO:5).

In embodiments, a 3E10 antibody or variant thereof, or antigen-binding fragment thereof described herein includes CDR sequences corresponding to the parent 3E10 antibody, with a known amino acid substitution in one or more CDR. Accordingly, in embodiments, a 3E10 antibody or variant thereof, or antigen-binding fragment thereof described herein includes one or more amino acid substitution, relative to the CDR sequences of the parent 3E10 or 3E10-D31N variant, selected from a G to S substitution at position 5 of VH CDR2, a T to S substitution at position 14 of VH CDR2, an S to T substitution at position 5 of VL CDR1, an M to L substitution at position 14 of VL CDR1, an H to A substitution at position 15 of VL CDR1, and an E to Q substitution at position 6 of VL CDR2.

Accordingly, in embodiments, a 3E10 antibody or variant thereof, or antigen-binding fragment thereof includes VH CDR2 comprising the amino acid sequence of 3E10-VH-CDR2.1 (SEQ ID NO:26) or 3E10-VH-CDR2.2 (SEQ ID NO:27). In embodiments, the 3E10 antibody or variant thereof, or antigen-binding fragment thereof further includes VL CDRs 1-3, and VH CDRs 1 and 3 according to the parent 3E10 antibody. In embodiments, the 3E10 antibody or variant thereof, or antigen-binding fragment thereof further includes VL CDRs 1-3, and VH CDRs 1 and 3 according to the 3E10- D31N variant. In embodiments, the 3E10 antibody or variant thereof, or antigen-binding fragment thereof further includes VL CDRs 1-3, and VH CDRs 1 and 3 having one or more amino acid substitutions relative to the CDRs of the parent 3E10 antibody or relative to the 3E10- D31N variant.

In embodiments, a 3E10 antibody or variant thereof, or antigen-binding fragment thereof includes VL CDR1 comprising the amino acid sequence of 3E10-VL-CDR1.1 (SEQ ID NO:28) or 3E10-VL-CDR1.2 (SEQ ID NO:29). In embodiments, the 3E10 antibody or variant thereof, or antigen-binding fragment thereof further includes VL CDRs 2 and 3, and VH CDRs 1-3 according to the parent 3E10 antibody. In embodiments, the 3E10 antibody or variant thereof, or antigen-binding fragment thereof further includes VL CDRs 2 and 3, and VH CDRs 1-3 according to the 3E10- D31N variant. In embodiments, the 3E10 antibody or variant thereof, or antigen-binding fragment thereof further includes VL CDRs 2 and 3, and VH CDRs 1-3 having one or more amino acid substitutions relative to the CDRs of the parent 3E10 antibody or relative to the 3E10- D31N variant.

In embodiments, a 3E10 antibody or variant thereof, or antigen-binding fragment thereof includes VL CDR2 comprising the amino acid sequence of 3E10-VL-CDR2.1 (SEQ ID NO:30). In embodiments, the 3E10 antibody or variant thereof, or antigen-binding fragment thereof further includes VL CDRs 1 and 3, and VH CDRs 1-3 according to the parent 3E10 antibody. In embodiments, the 3E10 antibody or variant thereof, or antigen-binding fragment thereof further includes VL CDRs 1 and 3, and VH CDRs 1-3 according to the 3E10- D31N variant. In embodiments, the 3E10 antibody or variant thereof, or antigen-binding fragment thereof further includes VL CDRs 1 and 3, and VH CDRs 1-3 having one or more amino acid substitutions relative to the CDRs of the parent 3E10 antibody or relative to the 3E10- D31N variant.

While some of the amino acid substitutions described above are fairly conservative substitutions—e.g., an S to T substitution at position 5 of VL CDR—other substitutions are to amino acids that have vastly different properties—e.g., an M to L substitution at position 14 of VL CDR1, an H to A substitution at position 15 of VL CDR1, and an E to Q substitution at position 6 of VL CDR2. This suggests, without being bound by theory, that at least these positions within the 3E10 CDR framework are tolerant to other amino acid substitutions.

In embodiments, a 3E10 antibody or variant thereof, or antigen-binding fragment thereof includes VH CDR2 comprising the amino acid sequence of 3E10-VH-CDR2.3 (SEQ ID NO:31). In embodiments, the 3E10 antibody or variant thereof, or antigen-binding fragment thereof further includes VL CDRs 1-3, and VH CDRs 1 and 3 according to the parent 3E10 antibody. In embodiments, the 3E10 antibody or variant thereof, or antigen-binding fragment thereof further includes VL CDRs 1-3, and VH CDRs 1 and 3 according to the 3E10- D31N variant. In embodiments, the 3E10 antibody or variant thereof, or antigen-binding fragment thereof further includes VL CDRs 1-3, and VH CDRs 1 and 3 having one or more amino acid substitutions relative to the CDRs of the parent 3E10 antibody or relative to the 3E10- D31N variant, e.g., as described herein.

In embodiments, a 3E10 antibody or variant thereof, or antigen-binding fragment thereof includes VL CDR1 comprising the amino acid sequence of 3E10-VL-CDR1.3 (SEQ ID NO:32). In embodiments, the 3E10 antibody or variant thereof, or antigen-binding fragment thereof further includes VL CDRs 2 and 3, and VH CDRs 1-3 according to the parent 3E10 antibody. In embodiments, the 3E10 antibody or variant thereof, or antigen-binding fragment thereof further includes VL CDRs 2 and 3, and VH CDRs 1-3 according to the 3E10- D31N variant. In embodiments, the 3E10 antibody or variant thereof, or antigen-binding fragment thereof further includes VL CDRs 2 and 3, and VH CDRs 1-3 having one or more amino acid substitutions relative to the CDRs of the parent 3E10 antibody or relative to the 3E10- D31N variant, e.g., as described herein.

In embodiments, a 3E10 antibody or variant thereof, or antigen-binding fragment thereof, includes VL CDR2 comprising the amino acid sequence of 3E10-VL-CDR2.2 (SEQ ID NO:33). In embodiments, the 3E10 antibody or variant thereof, or antigen-binding fragment thereof further includes VL CDRs 1 and 3, and VH CDRs 1-3 according to the parent 3E10 antibody. In embodiments, the 3E10 antibody or variant thereof, or antigen-binding fragment thereof further includes VL CDRs 1 and 3, and VH CDRs 1-3 according to the 3E10- D31N variant. In embodiments, the 3E10 antibody or variant thereof, or antigen-binding fragment thereof further includes VL CDRs 1 and 3, and VH CDRs 1-3 having one or more amino acid substitutions relative to the CDRs of the parent 3E10 antibody or relative to the 3E10- D31N variant, e.g., as described herein.

In embodiments, a 3E10 antibody or variant thereof, or antigen-binding fragment thereof includes VH CDR1 comprising the amino acid sequence of 3E10-VH-CDR1.c1 (SEQ ID NO:34), 3E10-VH-CDR1.c2 (SEQ ID NO:35), 3E10-VH-CDR1.c3 (SEQ ID NO:36), 3E10-VH-CDR1.c4 (SEQ ID NO:37), or 3E10-VH-CDR1.c5 (SEQ ID NO:38). In embodiments, the 3E10 antibody or variant thereof, or antigen-binding fragment thereof further includes VL CDRs 1-3, and VH CDRs 2 and 3 according to the parent 3E10 antibody. In embodiments, the 3E10 antibody or variant thereof, or antigen-binding fragment thereof further includes VL CDRs 1-3, and VH CDRs 2 and 3 having one or more amino acid substitutions relative to the CDRs of the parent 3E10 antibody, e.g., as described herein.

In embodiments, a 3E10 antibody or variant thereof, or antigen-binding fragment thereof includes VH CDR2 comprising the amino acid sequence of 3E10-VH-CDR2.c1 (SEQ ID NO:39), 3E10-VH-CDR2.c2 (SEQ ID NO:40), or 3E10-VH-CDR2.c3 (SEQ ID NO:41). In embodiments, the 3E10 antibody or variant thereof, or antigen-binding fragment thereof further includes VL CDRs 1-3, and VH CDRs 1 and 3 according to the parent 3E10 antibody. In embodiments, the 3E10 antibody or variant thereof, or antigen-binding fragment thereof further includes VL CDRs 1-3, and VH CDRs 1 and 3 according to the 3E10- D31N variant. In embodiments, the 3E10 antibody or variant thereof, or antigen-binding fragment thereof further includes VL CDRs 1-3, and VH CDRs 1 and 3 having one or more amino acid substitutions relative to the CDRs of the parent 3E10 antibody, e.g., as described herein.

In embodiments, a 3E10 antibody or variant thereof, or antigen-binding fragment thereof includes VH CDR3 comprising the amino acid sequence of 3E10-VH-CDR3.c1 (SEQ ID NO:42), 3E10-VH-CDR3.c2 (SEQ ID NO:43), or 3E10-VH-CDR3.c3 (SEQ ID NO:44). In embodiments, the 3E10 antibody or variant thereof, or antigen-binding fragment thereof further includes VL CDRs 1-3, and VH CDRs 1 and 2 according to the parent 3E10 antibody. In embodiments, the 3E10 antibody or variant thereof, or antigen-binding fragment thereof further includes VL CDRs 1-3, and VH CDRs 1 and 2 according to the 3E10- D31N variant. In embodiments, the 3E10 antibody or variant thereof, or antigen-binding fragment thereof further includes VL CDRs 1-3, and VH CDRs 1 and 2 having one or more amino acid substitutions relative to the CDRs of the parent 3E10 antibody, e.g., as described herein.

In embodiments, a 3E10 antibody or variant thereof, or antigen-binding fragment thereof includes VL CDR1 comprising the amino acid sequence of 3E10-VL-CDR1.c1 (SEQ ID NO:45), 3E10-VL-CDR1.c2 (SEQ ID NO:46), 3E10-VL-CDR1.c3 (SEQ ID NO:47), 3E10-VL-CDR1.c4 (SEQ ID NO:48), 3E10-VL-CDR1.c5 (SEQ ID NO:49), or 3E10-VL-CDR1.c6 (SEQ ID NO:50). In embodiments, the 3E10 antibody or variant thereof, or antigen-binding fragment thereof further includes VL CDRs 2 and 3, and VH CDRs 1-3 according to the parent 3E10 antibody. In embodiments, the 3E10 antibody or variant thereof, or antigen-binding fragment thereof further includes VL CDRs 2 and 3, and VH CDRs 1-3 according to the 3E10- D31N variant. In embodiments, the 3E10 antibody or variant thereof, or antigen-binding fragment thereof 8s further includes VL CDRs 2 and 3, and VH CDRs 1-3 having one or more amino acid substitutions relative to the CDRs of the parent 3E10 antibody, e.g., as described herein.

In embodiments, a 3E10 antibody or variant thereof, or antigen-binding fragment thereof includes VL CDR2 comprising the amino acid sequence of 3E10-VL-CDR2.c1 (SEQ ID NO:51). In embodiments, the 3E10 antibody or variant thereof, or antigen-binding fragment thereof further includes VL CDRs 1 and 3, and VH CDRs 1-3 according to the parent 3E10 antibody. In embodiments, the 3E10 antibody or variant thereof, or antigen-binding fragment thereof further includes VL CDRs 1 and 3, and VH CDRs 1-3 according to the 3E10- D31N variant. In embodiments, the 3E10 antibody or variant thereof, or antigen-binding fragment thereof further includes VL CDRs 1 and 3, and VH CDRs 1-3 having one or more amino acid substitutions relative to the CDRs of the parent 3E10 antibody, e.g., as described herein.

In embodiments, a 3E10 antibody or variant thereof, or antigen-binding fragment thereof includes VL CDR3 comprising the amino acid sequence of 3E10-VL-CDR3.c1 (SEQ ID NO:52), 3E10-VL-CDR3.c2 (SEQ ID NO:53), 3E10-VL-CDR3.c3 (SEQ ID NO:54), 3E10-VL-CDR3.c4 (SEQ ID NO:55), 3E10-VL-CDR3.c5 (SEQ ID NO:56), or 3E10-VL-CDR3.c6 (SEQ ID NO:57). In embodiments, the 3E10 antibody or variant thereof, or antigen-binding fragment thereof further includes VL CDRs 1 and 2, and VH CDRs 1-3 according to the parent 3E10 antibody. In embodiments, the 3E10 antibody or variant thereof, or antigen-binding fragment thereof further includes VL CDRs 1 and 2, and VH CDRs 1-3 according to the 3E10- D31N variant. In embodiments, the 3E10 antibody or variant thereof, or antigen-binding fragment thereof further includes VL CDRs 1 and 2, and VH CDRs 1-3 having one or more amino acid substitutions relative to the CDRs of the parent 3E10 antibody, e.g., as described herein.

It is also contemplated that a 3E10 antibody or variant thereof, or antigen-binding fragment thereof, as described herein, includes any combination of the 3E10 CDR amino acid substitutions described above.

Accordingly, in embodiments, a 3E10 antibody or variant thereof, or antigen-binding fragment thereof includes VH CDR1 comprising the amino acid sequence of 3E10-VH-CDR1m (SEQ ID NO:58). In embodiments, the 3E10 antibody or variant thereof, or antigen-binding fragment thereof further includes VL CDRs 1-3, and VH CDRs 2 and 3 according to the parent 3E10 antibody. In embodiments, the 3E10 antibody or variant thereof, or antigen-binding fragment thereof further includes VL CDRs 1-3, and VH CDRs 2 and 3 having one or more amino acid substitutions relative to the CDRs of the parent 3E10 antibody, e.g., as described herein.

In embodiments, a 3E10 antibody or variant thereof, or antigen-binding fragment thereof includes VH CDR2 comprising the amino acid sequence of 3E10-VH-CDR2m (SEQ ID NO:59). In embodiments, the 3E10 antibody or variant thereof, or antigen-binding fragment thereof further includes VL CDRs 1-3, and VH CDRs 1 and 3 according to the parent 3E10 antibody. In embodiments, the 3E10 antibody or variant thereof, or antigen-binding fragment thereof further includes VL CDRs 1-3, and VH CDRs 1 and 3 according to the 3E10-D31N variant. In embodiments, the 3E10 antibody or variant thereof, or antigen-binding fragment thereof further includes VL CDRs 1-3, and VH CDRs 1 and 3 having one or more amino acid substitutions relative to the CDRs of the parent 3E10 antibody, e.g., as described herein.

In embodiments, a 3E10 antibody or variant thereof, or antigen-binding fragment thereof includes VH CDR3 comprising the amino acid sequence of 3E10-VH-CDR3m (SEQ ID NO:60). In embodiments, the 3E10 antibody or variant thereof, or antigen-binding fragment thereof further includes VL CDRs 1-3, and VH CDRs 1 and 2 according to the parent 3E10 antibody. In embodiments, the 3E10 antibody or variant thereof, or antigen-binding fragment thereof further includes VL CDRs 1-3, and VH CDRs 1 and 2 according to the 3E10-D31N variant. In embodiments, the 3E10 antibody or variant thereof, or antigen-binding fragment thereof further includes VL CDRs 1-3, and VH CDRs 1 and 2 having one or more amino acid substitutions relative to the CDRs of the parent 3E10 antibody, e.g., as described herein.

In embodiments, a 3E10 antibody or variant thereof, or antigen-binding fragment thereof includes VL CDR1 comprising the amino acid sequence of 3E10-VL-CDR1m (SEQ ID NO:61). In embodiments, the 3E10 antibody or variant thereof, or antigen-binding fragment thereof further includes VL CDRs 2 and 3, and VH CDRs 1-3 according to the parent 3E10 antibody. In embodiments, the 3E10 antibody or variant thereof, or antigen-binding fragment thereof further includes VL CDRs 2 and 3, and VH CDRs 1-3 according to the 3E10-D31N variant. In embodiments, the 3E10 antibody or variant thereof, or antigen-binding fragment thereof further includes VL CDRs 2 and 3, and VH CDRs 1-3 having one or more amino acid substitutions relative to the CDRs of the parent 3E10 antibody, e.g., as described herein.

In embodiments, a 3E10 antibody or variant thereof, or antigen-binding fragment thereof includes VL CDR2 comprising the amino acid sequence of 3E10-VL-CDR2m (SEQ ID NO:62). In embodiments, the 3E10 antibody or variant thereof, or antigen-binding fragment thereof further includes VL CDRs 1 and 3, and VH CDRs 1-3 according to the parent 3E10 antibody. In embodiments, the 3E10 antibody or variant thereof, or antigen-binding fragment thereof further includes VL CDRs 1 and 3, and VH CDRs 1-3 according to the 3E10-D31N variant. In embodiments, the 3E10 antibody or variant thereof, or antigen-binding fragment thereof further includes VL CDRs 1 and 3, and VH CDRs 1-3 having one or more amino acid substitutions relative to the CDRs of the parent 3E10 antibody, e.g., as described herein.

In embodiments, a 3E10 antibody or variant thereof, or antigen-binding fragment thereof includes VL CDR3 comprising the amino acid sequence of 3E10-VL-CDR3m (SEQ ID NO:63). In embodiments, the 3E10 antibody or variant thereof, or antigen-binding fragment thereof further includes VL CDRs 1 and 2, and VH CDRs 1-3 according to the parent 3E10 antibody. In embodiments, the 3E10 antibody or variant thereof, or antigen-binding fragment thereof further includes VL CDRs 1 and 2, and VH CDRs 1-3 according to the 3E10-D31N variant. In embodiments, the 3E10 antibody or variant thereof, or antigen-binding fragment thereof further includes VL CDRs 1 and 2, and VH CDRs 1-3 having one or more amino acid substitutions relative to the CDRs of the parent 3E10 antibody, e.g., as described herein.

In embodiments, a 3E10 antibody or variant thereof, or antigen-binding fragment thereof described herein includes a VL CDR 1 comprising the amino acid sequence of 3E10-VL-CDR1m (SEQ ID NO:61), a VL CDR2 comprising the amino acid sequence of 3E10-VL-CDR2m (SEQ ID NO:62), a VL CDR3 comprising the amino acid sequence of 3E10-VL-CDR3m (SEQ ID NO:63), a heavy chain variable region (VH) CDR1 comprising the amino acid sequence of 3E10-VH-CDR1m (SEQ ID NO:58), a VH CDR2 comprising the amino acid sequence of 3E10-VH-CDR2m (SEQ ID NO:59), and a VH CDR3 comprising the amino acid sequence of 3E10-VH-CDR3m (SEQ ID NO:60).

In embodiments, a 3E10 antibody or variant thereof, or antigen-binding fragment thereof described herein refers to CDR sequences having no more than one amino acid substitution relative to the parent 3E10 antibody optionally including a D31N amino acid substitution in the VH CDR1. Accordingly, in embodiments, a 3E10 antibody or variant thereof, or antigen-binding fragment thereof includes a VL CDR 1 comprising an amino acid sequence having no more than one amino acid substitution relative to 3E10-VL-CDR1 (SEQ ID NO:9), a VL CDR2 comprising an amino acid sequence having no more than one amino acid substitution relative to 3E10-VL-CDR2 (SEQ ID NO:10), a VL CDR3 comprising an amino acid sequence having no more than one amino acid substitution relative to 3E10-VL-CDR3 (SEQ ID NO:11), a heavy chain variable region (VH) CDR1 comprising an amino acid sequence having no more than one amino acid substitution relative to 3E10-VH-CDR1a (SEQ ID NO:16), a VH CDR2 comprising an amino acid sequence having no more than one amino acid substitution relative to 3E10-VH-CDR2 (SEQ ID NO:4), and a VH CDR3 comprising an amino acid sequence having no more than one amino acid substitution relative to 3E10-VH-CDR3 (SEQ ID NO:5).

In embodiments, a 3E10 antibody or variant thereof, or antigen-binding fragment thereof described herein refers to CDR sequences having no more than two amino acid substitution relative to the parent 3E10 antibody optionally including a D31N amino acid substitution in the VH CDR1. Accordingly, in embodiments, a 3E10 antibody or variant thereof, or antigen-binding fragment thereof includes a VL CDR 1 comprising an amino acid sequence having no more than two amino acid substitutions relative to 3E10-VL-CDR1 (SEQ ID NO:9), a VL CDR2 comprising an amino acid sequence having no more than two amino acid substitutions relative to 3E10-VL-CDR2 (SEQ ID NO:10), a VL CDR3 comprising an amino acid sequence having no more than two amino acid substitutions relative to 3E10-VL-CDR3 (SEQ ID NO:11), a heavy chain variable region (VH) CDR1 comprising an amino acid sequence having no more than two amino acid substitutions relative to 3E10-VH-CDR1a (SEQ ID NO:16), a VH CDR2 comprising an amino acid sequence having no more than two amino acid substitutions relative to 3E10-VH-CDR2 (SEQ ID NO:4), and a VH CDR3 comprising an amino acid sequence having no more than two amino acid substitutions relative to 3E10-VH-CDR3 (SEQ ID NO:5).

Other variants of a 3E10 antibody or variant thereof, or antigen-binding fragment thereof are also known in the art, as disclosed for example, in Zack, et al., J. Immunol., 157(5):2082-8 (1996). For example, amino acid position 31 of the heavy chain variable region of 3E10 has been determined to be influential in the ability of the antibody and fragments thereof to penetrate nuclei and bind to DNA. A D31N mutation in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)). In embodiments, the antibody or antigen-binding fragment or variant described herein has the D31N substitution.

ADCs described herein can be prepared with any 3E10 antibodies or antigen-fragments thereof, or any humanized 3E10 antibodies or antigen-fragments thereof, disclosed in the prior art. See, for example WO 2015/106290, 2016/033324, WO 2019/018426, and WO 2019/018428 (each of which is specifically incorporated by reference herein, in its entirety).

In embodiments, an ADC comprises a humanized 3E10 antibody. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

In embodiments, the disclosure provides humanized antibodies, or antigen-binding fragments thereof, that incorporate any combination of the humanized VL and VH sequences disclosed here, as well as VL and VH sequences having sequence identity thereto, e.g., having at least about 90%, at least about 91%, at least about 92%, at least about 93%, at least about 94%, at least about 95%, at least about 96%, at least about 97%, at least about 98%, or at least about 99% identity to a VH or VL sequence described herein.

In embodiments, an ADC provided herein comprises a 3E10 antibody or antigen-binding fragment or variant thereof comprising one, two, or three CDRs of a heavy chain having the sequence of any one of SEQ ID NOs:1, 13, or 71-84. In embodiments, an ADC provided herein comprises a 3E10 antibody or antigen-binding fragment or variant thereof comprising one, two, or three CDRs of a heavy chain having the sequence of SEQ ID NO:1. In embodiments, an ADC provided herein comprises a 3E10 antibody or antigen-binding fragment or variant thereof comprising one, two, or three CDRs of a heavy chain having the sequence of SEQ ID NO:13. In embodiments, an ADC provided herein comprises a 3E10 antibody or antigen-binding fragment or variant thereof comprising one, two, or three CDRs of a heavy chain having the sequence of SEQ ID NO:71. In embodiments, an ADC provided herein comprises a 3E10 antibody or antigen-binding fragment or variant thereof comprising one, two, or three CDRs of a heavy chain having the sequence of SEQ ID NO:72. In embodiments, an ADC provided herein comprises a 3E10 antibody or antigen-binding fragment or variant thereof comprising one, two, or three CDRs of a heavy chain having the sequence of SEQ ID NO:73. In embodiments, an ADC provided herein comprises a 3E10 antibody or antigen-binding fragment or variant thereof comprising one, two, or three CDRs of a heavy chain having the sequence of SEQ ID NO:74. In embodiments, an ADC provided herein comprises a 3E10 antibody or antigen-binding fragment or variant thereof comprising one, two, or three CDRs of a heavy chain having the sequence of SEQ ID NO:75. In embodiments, an ADC provided herein comprises a 3E10 antibody or antigen-binding fragment or variant thereof comprising one, two, or three CDRs of a heavy chain having the sequence of SEQ ID NO:76. In embodiments, an ADC provided herein comprises a 3E10 antibody or antigen-binding fragment or variant thereof comprising one, two, or three CDRs of a heavy chain having the sequence of SEQ ID NO:77. In embodiments, an ADC provided herein comprises a 3E10 antibody or antigen-binding fragment or variant thereof comprising one, two, or three CDRs of a heavy chain having the sequence of SEQ ID NO:78. In embodiments, an ADC provided herein comprises a 3E10 antibody or antigen-binding fragment or variant thereof comprising one, two, or three CDRs of a heavy chain having the sequence of SEQ ID NO:79. In embodiments, an ADC provided herein comprises a 3E10 antibody or antigen-binding fragment or variant thereof comprising one, two, or three CDRs of a heavy chain having the sequence of SEQ ID NO:80. In embodiments, an ADC provided herein comprises a 3E10 antibody or antigen-binding fragment or variant thereof comprising one, two, or three CDRs of a heavy chain having the sequence of SEQ ID NO:81. In embodiments, an ADC provided herein comprises a 3E10 antibody or antigen-binding fragment or variant thereof comprising one, two, or three CDRs of a heavy chain having the sequence of SEQ ID NO:82. In embodiments, an ADC provided herein comprises a 3E10 antibody or antigen-binding fragment or variant thereof comprising one, two, or three CDRs of a heavy chain having the sequence of SEQ ID NO:83. In embodiments, an ADC provided herein comprises a 3E10 antibody or antigen-binding fragment or variant thereof comprising one, two, or three CDRs of a heavy chain having the sequence of SEQ ID NO:84.

In embodiments, an ADC provided herein comprises a 3E10 antibody or antigen-binding fragment or variant thereof comprising one, two, or three CDRs of a VH having the sequence of any one of SEQ ID NOs:2, 14, 64-70, 103-112. In embodiments, an ADC provided herein comprises a 3E10 antibody or antigen-binding fragment or variant thereof comprising one, two, or three CDRs of a VH having the sequence of SEQ ID NO:2. In embodiments, an ADC provided herein comprises a 3E10 antibody or antigen-binding fragment or variant thereof comprising one, two, or three CDRs of a VH having the sequence of SEQ ID NO:14. In embodiments, an ADC provided herein comprises a 3E10 antibody or antigen-binding fragment or variant thereof comprising one, two, or three CDRs of a VH having the sequence of SEQ ID NO:64. In embodiments, an ADC provided herein comprises a 3E10 antibody or antigen-binding fragment or variant thereof comprising one, two, or three CDRs of a VH having the sequence of SEQ ID NO:65. In embodiments, an ADC provided herein comprises a 3E10 antibody or antigen-binding fragment or variant thereof comprising one, two, or three CDRs of a VH having the sequence of SEQ ID NO:66. In embodiments, an ADC provided herein comprises a 3E10 antibody or antigen-binding fragment or variant thereof comprising one, two, or three CDRs of a VH having the sequence of SEQ ID NO:67. In embodiments, an ADC provided herein comprises a 3E10 antibody or antigen-binding fragment or variant thereof comprising one, two, or three CDRs of a VH having the sequence of SEQ ID NO:68. In embodiments, an ADC provided herein comprises a 3E10 antibody or antigen-binding fragment or variant thereof comprising one, two, or three CDRs of a VH having the sequence of SEQ ID NO:69. In embodiments, an ADC provided herein comprises a 3E10 antibody or antigen-binding fragment or variant thereof comprising one, two, or three CDRs of a VH having the sequence of SEQ ID NO:70. In embodiments, an ADC provided herein comprises a 3E10 antibody or antigen-binding fragment or variant thereof comprising one, two, or three CDRs of a VH having the sequence of SEQ ID NO:103. In embodiments, an ADC provided herein comprises a 3E10 antibody or antigen-binding fragment or variant thereof comprising one, two, or three CDRs of a VH having the sequence of SEQ ID NO:104. In embodiments, an ADC provided herein comprises a 3E10 antibody or antigen-binding fragment or variant thereof comprising one, two, or three CDRs of a VH having the sequence of SEQ ID NO:105. In embodiments, an ADC provided herein comprises a 3E10 antibody or antigen-binding fragment or variant thereof comprising one, two, or three CDRs of a VH having the sequence of SEQ ID NO:106. In embodiments, an ADC provided herein comprises a 3E10 antibody or antigen-binding fragment or variant thereof comprising one, two, or three CDRs of a VH having the sequence of SEQ ID NO:107. In embodiments, an ADC provided herein comprises a 3E10 antibody or antigen-binding fragment or variant thereof comprising one, two, or three CDRs of a VH having the sequence of SEQ ID NO:108. In embodiments, an ADC provided herein comprises a 3E10 antibody or antigen-binding fragment or variant thereof comprising one, two, or three CDRs of a VH having the sequence of SEQ ID NO:109. In embodiments, an ADC provided herein comprises a 3E10 antibody or antigen-binding fragment or variant thereof comprising one, two, or three CDRs of a VH having the sequence of SEQ ID NO:110. In embodiments, an ADC provided herein comprises a 3E10 antibody or antigen-binding fragment or variant thereof comprising one, two, or three CDRs of a VH having the sequence of SEQ ID NO:111. In embodiments, an ADC provided herein comprises a 3E10 antibody or antigen-binding fragment or variant thereof comprising one, two, or three CDRs of a VH having the sequence of SEQ ID NO:112.

In embodiments, an ADC provided herein comprises a 3E10 antibody or antigen-binding fragment or variant thereof comprising one, two, or three CDRs of a light chain having the sequence of any one of SEQ ID NOs:7, 20, or 91-102. In embodiments, an ADC provided herein comprises a 3E10 antibody or antigen-binding fragment or variant thereof comprising one, two, or three CDRs of a light chain having the sequence of SEQ ID NO:7. In embodiments, an ADC provided herein comprises a 3E10 antibody or antigen-binding fragment or variant thereof comprising one, two, or three CDRs of a light chain having the sequence of SEQ ID NO:20. In embodiments, an ADC provided herein comprises a 3E10 antibody or antigen-binding fragment or variant thereof comprising one, two, or three CDRs of a light chain having the sequence of SEQ ID NO:91. In embodiments, an ADC provided herein comprises a 3E10 antibody or antigen-binding fragment or variant thereof comprising one, two, or three CDRs of a light chain having the sequence of SEQ ID NO:92. In embodiments, an ADC provided herein comprises a 3E10 antibody or antigen-binding fragment or variant thereof comprising one, two, or three CDRs of a light chain having the sequence of SEQ ID NO:93. In embodiments, an ADC provided herein comprises a 3E10 antibody or antigen-binding fragment or variant thereof comprising one, two, or three CDRs of a light chain having the sequence of SEQ ID NO:94. In embodiments, an ADC provided herein comprises a 3E10 antibody or antigen-binding fragment or variant thereof comprising one, two, or three CDRs of a light chain having the sequence of SEQ ID NO:95. In embodiments, an ADC provided herein comprises a 3E10 antibody or antigen-binding fragment or variant thereof comprising one, two, or three CDRs of a light chain having the sequence of SEQ ID NO:96. In embodiments, an ADC provided herein comprises a 3E10 antibody or antigen-binding fragment or variant thereof comprising one, two, or three CDRs of a light chain having the sequence of SEQ ID NO:97. In embodiments, an ADC provided herein comprises a 3E10 antibody or antigen-binding fragment or variant thereof comprising one, two, or three CDRs of a light chain having the sequence of SEQ ID NO:98. In embodiments, an ADC provided herein comprises a 3E10 antibody or antigen-binding fragment or variant thereof comprising one, two, or three CDRs of a light chain having the sequence of SEQ ID NO:99. In embodiments, an ADC provided herein comprises a 3E10 antibody or antigen-binding fragment or variant thereof comprising one, two, or three CDRs of a light chain having the sequence of SEQ ID NO:100. In embodiments, an ADC provided herein comprises a 3E10 antibody or antigen-binding fragment or variant thereof comprising one, two, or three CDRs of a light chain having the sequence of SEQ ID NO:101. In embodiments, an ADC provided herein comprises a 3E10 antibody or antigen-binding fragment or variant thereof comprising one, two, or three CDRs of a light chain having the sequence of SEQ ID NO:102.

In embodiments, an ADC provided herein comprises a 3E10 antibody or antigen-binding fragment or variant thereof comprising one, two, or three CDRs of a VL having the sequence of any one of SEQ ID NOs:8, 21, 85-90, or 113-121. In embodiments, an ADC provided herein comprises a 3E10 antibody or antigen-binding fragment or variant thereof comprising one, two, or three CDRs of a VL having the sequence of SEQ ID NO:8. In embodiments, an ADC provided herein comprises a 3E10 antibody or antigen-binding fragment or variant thereof comprising one, two, or three CDRs of a VL having the sequence of SEQ ID NO:21. In embodiments, an ADC provided herein comprises a 3E10 antibody or antigen-binding fragment or variant thereof comprising one, two, or three CDRs of a VL having the sequence of SEQ ID NO:85. In embodiments, an ADC provided herein comprises a 3E10 antibody or antigen-binding fragment or variant thereof comprising one, two, or three CDRs of a VL having the sequence of SEQ ID NO:86. In embodiments, an ADC provided herein comprises a 3E10 antibody or antigen-binding fragment or variant thereof comprising one, two, or three CDRs of a VL having the sequence of SEQ ID NO:87. In embodiments, an ADC provided herein comprises a 3E10 antibody or antigen-binding fragment or variant thereof comprising one, two, or three CDRs of a VL having the sequence of SEQ ID NO:88. In embodiments, an ADC provided herein comprises a 3E10 antibody or antigen-binding fragment or variant thereof comprising one, two, or three CDRs of a VL having the sequence of SEQ ID NO:89. In embodiments, an ADC provided herein comprises a 3E10 antibody or antigen-binding fragment or variant thereof comprising one, two, or three CDRs of a VL having the sequence of SEQ ID NO:90. In embodiments, an ADC provided herein comprises a 3E10 antibody or antigen-binding fragment or variant thereof comprising one, two, or three CDRs of a VL having the sequence of SEQ ID NO:113. In embodiments, an ADC provided herein comprises a 3E10 antibody or antigen-binding fragment or variant thereof comprising one, two, or three CDRs of a VL having the sequence of SEQ ID NO:114. In embodiments, an ADC provided herein comprises a 3E10 antibody or antigen-binding fragment or variant thereof comprising one, two, or three CDRs of a VL having the sequence of SEQ ID NO:115. In embodiments, an ADC provided herein comprises a 3E10 antibody or antigen-binding fragment or variant thereof comprising one, two, or three CDRs of a VL having the sequence of SEQ ID NO:116. In embodiments, an ADC provided herein comprises a 3E10 antibody or antigen-binding fragment or variant thereof comprising one, two, or three CDRs of a VL having the sequence of SEQ ID NO:117. In embodiments, an ADC provided herein comprises a 3E10 antibody or antigen-binding fragment or variant thereof comprising one, two, or three CDRs of a VL having the sequence of SEQ ID NO:118. In embodiments, an ADC provided herein comprises a 3E10 antibody or antigen-binding fragment or variant thereof comprising one, two, or three CDRs of a VL having the sequence of SEQ ID NO:119. In embodiments, an ADC provided herein comprises a 3E10 antibody or antigen-binding fragment or variant thereof comprising one, two, or three CDRs of a VL having the sequence of SEQ ID NO:120. In embodiments, an ADC provided herein comprises a 3E10 antibody or antigen-binding fragment or variant thereof comprising one, two, or three CDRs of a VL having the sequence of SEQ ID NO:121.

In embodiments, an ADC provided herein comprises a 3E10 antibody or antigen-binding fragment or variant thereof comprising one, two, three, four, five, or six CDRs of an scFv having the sequence of any one of SEQ ID NOs:122-137. In embodiments, an ADC provided herein comprises a 3E10 antibody or antigen-binding fragment or variant thereof comprising one, two, three, four, five, or six CDRs of an scFv having the sequence of SEQ ID NO:122. In embodiments, an ADC provided herein comprises a 3E10 antibody or antigen-binding fragment or variant thereof comprising one, two, three, four, five, or six CDRs of an scFv having the sequence of SEQ ID NO:123. In embodiments, an ADC provided herein comprises a 3E10 antibody or antigen-binding fragment or variant thereof comprising one, two, three, four, five, or six CDRs of an scFv having the sequence of SEQ ID NO:124. In embodiments, an ADC provided herein comprises a 3E10 antibody or antigen-binding fragment or variant thereof comprising one, two, three, four, five, or six CDRs of an scFv having the sequence of SEQ ID NO:125. In embodiments, an ADC provided herein comprises a 3E10 antibody or antigen-binding fragment or variant thereof comprising one, two, three, four, five, or six CDRs of an scFv having the sequence of SEQ ID NO:126. In embodiments, an ADC provided herein comprises a 3E10 antibody or antigen-binding fragment or variant thereof comprising one, two, three, four, five, or six CDRs of an scFv having the sequence of SEQ ID NO:127. In embodiments, an ADC provided herein comprises a 3E10 antibody or antigen-binding fragment or variant thereof comprising one, two, three, four, five, or six CDRs of an scFv having the sequence of SEQ ID NO:128. In embodiments, an ADC provided herein comprises a 3E10 antibody or antigen-binding fragment or variant thereof comprising one, two, three, four, five, or six CDRs of an scFv having the sequence of SEQ ID NO:129. In embodiments, an ADC provided herein comprises a 3E10 antibody or antigen-binding fragment or variant thereof comprising one, two, three, four, five, or six CDRs of an scFv having the sequence of SEQ ID NO:130. In embodiments, an ADC provided herein comprises a 3E10 antibody or antigen-binding fragment or variant thereof comprising one, two, three, four, five, or six CDRs of an scFv having the sequence of SEQ ID NO:131. In embodiments, an ADC provided herein comprises a 3E10 antibody or antigen-binding fragment or variant thereof comprising one, two, three, four, five, or six CDRs of an scFv having the sequence of SEQ ID NO:132. In embodiments, an ADC provided herein comprises a 3E10 antibody or antigen-binding fragment or variant thereof comprising one, two, three, four, five, or six CDRs of an scFv having the sequence of SEQ ID NO:133. In embodiments, an ADC provided herein comprises a 3E10 antibody or antigen-binding fragment or variant thereof comprising one, two, three, four, five, or six CDRs of an scFv having the sequence of SEQ ID NO:134. In embodiments, an ADC provided herein comprises a 3E10 antibody or antigen-binding fragment or variant thereof comprising one, two, three, four, five, or six CDRs of an scFv having the sequence of SEQ ID NO:135. In embodiments, an ADC provided herein comprises a 3E10 antibody or antigen-binding fragment or variant thereof comprising one, two, three, four, five, or six CDRs of an scFv having the sequence of SEQ ID NO:136. In embodiments, an ADC provided herein comprises a 3E10 antibody or antigen-binding fragment or variant thereof comprising one, two, three, four, five, or six CDRs of an scFv having the sequence of SEQ ID NO:137.

In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to any one of SEQ ID NOs:1, 13, or 71-84. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:1. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:13. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:71. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:72. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:73. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:74. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:75. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:76. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:77. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:78. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:79. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:80. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:81. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:82. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:83. In embodiments, an ADC provided herein comprises a 3E10 antibody or antigen-binding fragment or variant thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:84.

In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to any one of SEQ ID NOs:7, 20, or 91-102. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:7. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:20. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:91. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:92. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:93. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:94. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:95. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:96. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:97. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:98. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:99. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:100. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:101. In embodiments, an ADC provided herein comprises a 3E10 antibody or antigen-binding fragment or variant thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:102.

In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to any one of SEQ ID NOs: 2, 14, 64-70, or 103-112. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:2. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:14. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:64. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:65. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:66. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:67. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:68. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:69. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:70. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:103. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:104. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:105. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:106. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:107. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:108. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:109. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:110. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:111. In embodiments, an ADC provided herein comprises a 3E10 antibody or antigen-binding fragment or variant thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:112.

In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to any one of SEQ ID NOs: 8, 21, 85-90, or 113-121. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:8. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:21. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:85. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:86. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:87. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:88. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:89. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:90. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:113. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:114. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:115. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:116. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:117. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:118. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:119. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:120. In embodiments, an ADC provided herein comprises a 3E10 antibody or antigen-binding fragment or variant thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:121.

In embodiments, an ADC provided herein comprises a humanized 3E10 antibody, or antigen-binding fragment thereof, comprising a light chain variable domain (3E10-VL) comprising an amino acid sequence that is at least about 97% identical to an amino acid sequence selected from the group consisting of 3E10-VL-h1 (SEQ ID NO:85), 3E10-VL-h2 (SEQ ID NO:86), 3E10-VL-h3 (SEQ ID NO:87), 3E10-VL-h4 (SEQ ID NO:88), 3E10-VL-h5 (SEQ ID NO:89), and 3E10-VL-h6 (SEQ ID NO:90) and a heavy chain variable domain (3E10-VH) comprising an amino acid sequence that is at least about 95% identical to an amino acid sequence selected from the group consisting of 3E10-VH-h1 (SEQ ID NO:64), 3E10-VH-h2 (SEQ ID NO:65), 3E10-VH-h3 (SEQ ID NO:66), 3E10-VH-h4 (SEQ ID NO:67), 3E10-VH-h5 (SEQ ID NO:68), 3E10-VH-h6 (SEQ ID NO:69), and 3E10-VH-h7 (SEQ ID NO:70).

In embodiments, the sequence of the 3E10-VL is at least about 97% identical to 3E10-VL-h1 (SEQ ID NO:85). In embodiments, the sequence of the 3E10-VL is at least about 98% identical to 3E10-VL-h1 (SEQ ID NO:85). In embodiments, the sequence of the 3E10-VL is at least about 99% identical to 3E10-VL-h1 (SEQ ID NO:85). In embodiments, the sequence of the 3E10-VL is 3E10-VL-h1 (SEQ ID NO:85).

In embodiments, the sequence of the 3E10-VL is at least about 97% identical to 3E10-VL-h2 (SEQ ID NO:86). In embodiments, the sequence of the 3E10-VL is at least about 98% identical to 3E10-VL-h2 (SEQ ID NO:86). In embodiments, the sequence of the 3E10-VL is at least about 99% identical to 3E10-VL-h2 (SEQ ID NO:86). In embodiments, the sequence of the 3E10-VL is 3E10-VL-h2 (SEQ ID NO:86).

In embodiments, the sequence of the 3E10-VL is at least about 97% identical to 3E10-VL-h3 (SEQ ID NO:87). In embodiments, the sequence of the 3E10-VL is at least about 98% identical to 3E10-VL-h3 (SEQ ID NO:87). In embodiments, the sequence of the 3E10-VL is at least about 99% identical to 3E10-VL-h3 (SEQ ID NO:87). In embodiments, the sequence of the 3E10-VL is 3E10-VL-h3 (SEQ ID NO:87).

In embodiments, the sequence of the 3E10-VL is at least about 97% identical to 3E10-VL-h4 (SEQ ID NO:88). In embodiments, the sequence of the 3E10-VL is at least about 98% identical to 3E10-VL-h4 (SEQ ID NO:88). In embodiments, the sequence of the 3E10-VL is at least about 99% identical to 3E10-VL-h4 (SEQ ID NO:88). In embodiments, the sequence of the 3E10-VL is 3E10-VL-h4 (SEQ ID NO:88).

In embodiments, the sequence of the 3E10-VL is at least about 97% identical to 3E10-VL-h5 (SEQ ID NO:89). In embodiments, the sequence of the 3E10-VL is at least about 98% identical to 3E10-VL-h5 (SEQ ID NO:89). In embodiments, the sequence of the 3E10-VL is at least about 99% identical to 3E10-VL-h5 (SEQ ID NO:89). In embodiments, the sequence of the 3E10-VL is 3E10-VL-h5 (SEQ ID NO:89).

In embodiments, the sequence of the 3E10-VL is at least about 97% identical to 3E10-VL-h6 (SEQ ID NO:90). In embodiments, the sequence of the 3E10-VL is at least about 98% identical to 3E10-VL-h6 (SEQ ID NO:90). In embodiments, the sequence of the 3E10-VL is at least about 99% identical to 3E10-VL-h6 (SEQ ID NO:90). In embodiments, the sequence of the 3E10-VL is 3E10-VL-h6 (SEQ ID NO:90).

In embodiments, the sequence of the 3E10-VH is at least about 95% identical to 3E10-VH-h1 (SEQ ID NO:64). In embodiments, the sequence of the 3E10-VH is at least about 96% identical to 3E10-VH-h1 (SEQ ID NO:64). In embodiments, the sequence of the 3E10-VH is at least about 97% identical to 3E10-VH-h1 (SEQ ID NO:64). In embodiments, the sequence of the 3E10-VH is at least about 98% identical to 3E10-VH-h1 (SEQ ID NO:64). In embodiments, the sequence of the 3E10-VH is at least about 99% identical to 3E10-VH-h1 (SEQ ID NO:64). In embodiments, the sequence of the 3E10-VH is 3E10-VH-h1 (SEQ ID NO:64).

In embodiments, the sequence of the 3E10-VH is at least about 95% identical to 3E10-VH-h2 (SEQ ID NO:65). In embodiments, the sequence of the 3E10-VH is at least about 96% identical to 3E10-VH-h2 (SEQ ID NO:65). In embodiments, the sequence of the 3E10-VH is at least about 97% identical to 3E10-VH-h2 (SEQ ID NO:65). In embodiments, the sequence of the 3E10-VH is at least about 98% identical to 3E10-VH-h2 (SEQ ID NO:65). In embodiments, the sequence of the 3E10-VH is at least about 99% identical to 3E10-VH-h2 (SEQ ID NO:65). In embodiments, the sequence of the 3E10-VH is 3E10-VH-h2 (SEQ ID NO:65).

In embodiments, the sequence of the 3E10-VH is at least about 95% identical to 3E10-VH-h3 (SEQ ID NO:66). In embodiments, the sequence of the 3E10-VH is at least about 96% identical to 3E10-VH-h3 (SEQ ID NO:66). In embodiments, the sequence of the 3E10-VH is at least about 97% identical to 3E10-VH-h3 (SEQ ID NO:66). In embodiments, the sequence of the 3E10-VH is at least about 98% identical to 3E10-VH-h3 (SEQ ID NO:66). In embodiments, the sequence of the 3E10-VH is at least about 99% identical to 3E10-VH-h3 (SEQ ID NO:66). In embodiments, the sequence of the 3E10-VH is 3E10-VH-h3 (SEQ ID NO:66).

In embodiments, the sequence of the 3E10-VH is at least about 95% identical to 3E10-VH-h4 (SEQ ID NO:67). In embodiments, the sequence of the 3E10-VH is at least about 96% identical to 3E10-VH-h4 (SEQ ID NO:67). In embodiments, the sequence of the 3E10-VH is at least about 97% identical to 3E10-VH-h4 (SEQ ID NO:67). In embodiments, the sequence of the 3E10-VH is at least about 98% identical to 3E10-VH-h4 (SEQ ID NO:67). In embodiments, the sequence of the 3E10-VH is at least about 99% identical to 3E10-VH-h4 (SEQ ID NO:67). In embodiments, the sequence of the 3E10-VH is 3E10-VH-h4 (SEQ ID NO:67).

In embodiments, the sequence of the 3E10-VH is at least about 95% identical to 3E10-VH-h5 (SEQ ID NO:68). In embodiments, the sequence of the 3E10-VH is at least about 96% identical to 3E10-VH-h5 (SEQ ID NO:68). In embodiments, the sequence of the 3E10-VH is at least about 97% identical to 3E10-VH-h5 (SEQ ID NO:68). In embodiments, the sequence of the 3E10-VH is at least about 98% identical to 3E10-VH-h5 (SEQ ID NO:68). In embodiments, the sequence of the 3E10-VH is at least about 99% identical to 3E10-VH-h5 (SEQ ID NO:68). In embodiments, the sequence of the 3E10-VH is 3E10-VH-h5 (SEQ ID NO:68).

In embodiments, the sequence of the 3E10-VH is at least about 95% identical to 3E10-VH-h6 (SEQ ID NO:69). In embodiments, the sequence of the 3E10-VH is at least about 96% identical to 3E10-VH-h6 (SEQ ID NO:69). In embodiments, the sequence of the 3E10-VH is at least about 97% identical to 3E10-VH-h6 (SEQ ID NO:69). In embodiments, the sequence of the 3E10-VH is at least about 98% identical to 3E10-VH-h6 (SEQ ID NO:69). In embodiments, the sequence of the 3E10-VH is at least about 99% identical to 3E10-VH-h6 (SEQ ID NO:69). In embodiments, the sequence of the 3E10-VH is 3E10-VH-h6 (SEQ ID NO:69).

In embodiments, the sequence of the 3E10-VH is at least about 95% identical to 3E10-VH-h7 (SEQ ID NO:70). In embodiments, the sequence of the 3E10-VH is at least about 96% identical to 3E10-VH-h7 (SEQ ID NO:70). In embodiments, the sequence of the 3E10-VH is at least about 97% identical to 3E10-VH-h7 (SEQ ID NO:70). In embodiments, the sequence of the 3E10-VH is at least about 98% identical to 3E10-VH-h7 (SEQ ID NO:70). In embodiments, the sequence of the 3E10-VH is at least about 99% identical to 3E10-VH-h7 (SEQ ID NO:70). In embodiments, the sequence of the 3E10-VH is 3E10-VH-h7 (SEQ ID NO:70).

In embodiments, an ADC comprising a humanized 3E10 antibody, or antigen-binding fragment thereof, described herein includes a light chain (3E10-LC) comprising an amino acid sequence that is at least about 95% identical to an amino acid sequence selected from the group consisting of 3E10-LC-h1m (SEQ ID NO:91), 3E10-LC-h2m (SEQ ID NO:92), 3E10-LC-h3m (SEQ ID NO:93), 3E10-LC-h4m (SEQ ID NO:94), 3E10-LC-h5m (SEQ ID NO:95), and 3E10-LC-h6m (SEQ ID NO:96) and a heavy chain (3E10-HC) comprising an amino acid sequence that is at least about 95% identical to an amino acid sequence selected from the group consisting of 3E10-HC-h1m (SEQ ID NO:71), 3E10-HC-h2m (SEQ ID NO:72), 3E10-HC-h3m (SEQ ID NO:73), 3E10-HC-h4m (SEQ ID NO:74), 3E10-HC-h5m (SEQ ID NO:75), 3E10-HC-h6m (SEQ ID NO:76), and 3E10-HC-h7m (SEQ ID NO:77).

In embodiments, the sequence of the 3E10-LC is at least about 95% identical to 3E10-LC-h1m (SEQ ID NO:91). In embodiments, the sequence of the 3E10-LC is at least about 96% identical to 3E10-LC-h1m (SEQ ID NO:91). In embodiments, the sequence of the 3E10-LC is at least about 97% identical to 3E10-LC-h1m (SEQ ID NO:91). In embodiments, the sequence of the 3E10-LC is at least about 98% identical to 3E10-LC-h1m (SEQ ID NO:91). In embodiments, the sequence of the 3E10-LC is at least about 99% identical to 3E10-LC-h1m (SEQ ID NO:91). In embodiments, the sequence of the 3E10-LC is 3E10-LC-h1m (SEQ ID NO:91).

In embodiments, the sequence of the 3E10-LC is at least about 95% identical to 3E10-LC-h2m (SEQ ID NO:92). In embodiments, the sequence of the 3E10-LC is at least about 96% identical to 3E10-LC-h2m (SEQ ID NO:92). In embodiments, the sequence of the 3E10-LC is at least about 97% identical to 3E10-LC-h2m (SEQ ID NO:92). In embodiments, the sequence of the 3E10-LC is at least about 98% identical to 3E10-LC-h2m (SEQ ID NO:92). In embodiments, the sequence of the 3E10-LC is at least about 99% identical to 3E10-LC-h2m (SEQ ID NO:92). In embodiments, the sequence of the 3E10-LC is 3E10-LC-h2m (SEQ ID NO:92).

In embodiments, the sequence of the 3E10-LC is at least about 95% identical to 3E10-LC-h3m (SEQ ID NO:93). In embodiments, the sequence of the 3E10-LC is at least about 96% identical to 3E10-LC-h3m (SEQ ID NO:93). In embodiments, the sequence of the 3E10-LC is at least about 97% identical to 3E10-LC-h3m (SEQ ID NO:93). In embodiments, the sequence of the 3E10-LC is at least about 98% identical to 3E10-LC-h3m (SEQ ID NO:93). In embodiments, the sequence of the 3E10-LC is at least about 99% identical to 3E10-LC-h3m (SEQ ID NO:93). In embodiments, the sequence of the 3E10-LC is 3E10-LC-h3m (SEQ ID NO:93).

In embodiments, the sequence of the 3E10-LC is at least about 95% identical to 3E10-LC-h4m (SEQ ID NO:94). In embodiments, the sequence of the 3E10-LC is at least about 96% identical to 3E10-LC-h4m (SEQ ID NO:94). In embodiments, the sequence of the 3E10-LC is at least about 97% identical to 3E10-LC-h4m (SEQ ID NO:94). In embodiments, the sequence of the 3E10-LC is at least about 98% identical to 3E10-LC-h4m (SEQ ID NO:94). In embodiments, the sequence of the 3E10-LC is at least about 99% identical to 3E10-LC-h4m (SEQ ID NO:94). In embodiments, the sequence of the 3E10-LC is 3E10-LC-h4m (SEQ ID NO:94).

In embodiments, the sequence of the 3E10-LC is at least about 95% identical to 3E10-LC-h5m (SEQ ID NO:95). In embodiments, the sequence of the 3E10-LC is at least about 96% identical to 3E10-LC-h5m (SEQ ID NO:95). In embodiments, the sequence of the 3E10-LC is at least about 97% identical to 3E10-LC-h5m (SEQ ID NO:95). In embodiments, the sequence of the 3E10-LC is at least about 98% identical to 3E10-LC-h5m (SEQ ID NO:95). In embodiments, the sequence of the 3E10-LC is at least about 99% identical to 3E10-LC-h5m (SEQ ID NO:95). In embodiments, the sequence of the 3E10-LC is 3E10-LC-h5m (SEQ ID NO:95).

In embodiments, the sequence of the 3E10-LC is at least about 95% identical to 3E10-LC-h6m (SEQ ID NO:96). In embodiments, the sequence of the 3E10-LC is at least about 96% identical to 3E10-LC-h6m (SEQ ID NO:96). In embodiments, the sequence of the 3E10-LC is at least about 97% identical to 3E10-LC-h6m (SEQ ID NO:96). In embodiments, the sequence of the 3E10-LC is at least about 98% identical to 3E10-LC-h6m (SEQ ID NO:96). In embodiments, the sequence of the 3E10-LC is at least about 99% identical to 3E10-LC-h6m (SEQ ID NO:96). In embodiments, the sequence of the 3E10-LC is 3E10-LC-h6m (SEQ ID NO:96).

In embodiments, the sequence of the 3E10-HC is at least about 95% identical to 3E10-HC-h1m (SEQ ID NO:71). In embodiments, the sequence of the 3E10-HC is at least about 96% identical to 3E10-HC-h1m (SEQ ID NO:71). In embodiments, the sequence of the 3E10-HC is at least about 97% identical to 3E10-HC-h1m (SEQ ID NO:71). In embodiments, the sequence of the 3E10-HC is at least about 98% identical to 3E10-HC-h1m (SEQ ID NO:71). In embodiments, the sequence of the 3E10-HC is at least about 99% identical to 3E10-HC-h1m (SEQ ID NO:71). In embodiments, the sequence of the 3E10-HC is 3E10-HC-h1m (SEQ ID NO:71).

In embodiments, the sequence of the 3E10-HC is at least about 95% identical to 3E10-HC-h2m (SEQ ID NO:72). In embodiments, the sequence of the 3E10-HC is at least about 96% identical to 3E10-HC-h2m (SEQ ID NO:72). In embodiments, the sequence of the 3E10-HC is at least about 97% identical to 3E10-HC-h2m (SEQ ID NO:72). In embodiments, the sequence of the 3E10-HC is at least about 98% identical to 3E10-HC-h2m (SEQ ID NO:72). In embodiments, the sequence of the 3E10-HC is at least about 99% identical to 3E10-HC-h2m (SEQ ID NO:72). In embodiments, the sequence of the 3E10-HC is 3E10-HC-h2m (SEQ ID NO:72).

In embodiments, the sequence of the 3E10-HC is at least about 95% identical to 3E10-HC-h3m (SEQ ID NO:73). In embodiments, the sequence of the 3E10-HC is at least about 96% identical to 3E10-HC-h3m (SEQ ID NO:73). In embodiments, the sequence of the 3E10-HC is at least about 97% identical to 3E10-HC-h3m (SEQ ID NO:73). In embodiments, the sequence of the 3E10-HC is at least about 98% identical to 3E10-HC-h3m (SEQ ID NO:73). In embodiments, the sequence of the 3E10-HC is at least about 99% identical to 3E10-HC-h3m (SEQ ID NO:73). In embodiments, the sequence of the 3E10-HC is 3E10-HC-h3m (SEQ ID NO:73).

In embodiments, the sequence of the 3E10-HC is at least about 95% identical to 3E10-HC-h4m (SEQ ID NO:74). In embodiments, the sequence of the 3E10-HC is at least about 96% identical to 3E10-HC-h4m (SEQ ID NO:74). In embodiments, the sequence of the 3E10-HC is at least about 97% identical to 3E10-HC-h4m (SEQ ID NO:74). In embodiments, the sequence of the 3E10-HC is at least about 98% identical to 3E10-HC-h4m (SEQ ID NO:74). In embodiments, the sequence of the 3E10-HC is at least about 99% identical to 3E10-HC-h4m (SEQ ID NO:74). In embodiments, the sequence of the 3E10-HC is 3E10-HC-h4m (SEQ ID NO:74).

In embodiments, the sequence of the 3E10-HC is at least about 95% identical to 3E10-HC-h5m (SEQ ID NO:75). In embodiments, the sequence of the 3E10-HC is at least about 96% identical to 3E10-HC-h5m (SEQ ID NO:75). In embodiments, the sequence of the 3E10-HC is at least about 97% identical to 3E10-HC-h5m (SEQ ID NO:75). In embodiments, the sequence of the 3E10-HC is at least about 98% identical to 3E10-HC-h5m (SEQ ID NO:75). In embodiments, the sequence of the 3E10-HC is at least about 99% identical to 3E10-HC-h5m (SEQ ID NO:75). In embodiments, the sequence of the 3E10-HC is 3E10-HC-h5m (SEQ ID NO:75).

In embodiments, the sequence of the 3E10-HC is at least about 95% identical to 3E10-HC-h6m (SEQ ID NO:76). In embodiments, the sequence of the 3E10-HC is at least about 96% identical to 3E10-HC-h6m (SEQ ID NO:76). In embodiments, the sequence of the 3E10-HC is at least about 97% identical to 3E10-HC-h6m (SEQ ID NO:76). In embodiments, the sequence of the 3E10-HC is at least about 98% identical to 3E10-HC-h6m (SEQ ID NO:76). In embodiments, the sequence of the 3E10-HC is at least about 99% identical to 3E10-HC-h6m (SEQ ID NO:76). In embodiments, the sequence of the 3E10-HC is 3E10-HC-h6m (SEQ ID NO:76).

In embodiments, the sequence of the 3E10-HC is at least about 95% identical to 3E10-HC-h7m (SEQ ID NO:77). In embodiments, the sequence of the 3E10-HC is at least about 96% identical to 3E10-HC-h7m (SEQ ID NO:77). In embodiments, the sequence of the 3E10-HC is at least about 97% identical to 3E10-HC-h7m (SEQ ID NO:77). In embodiments, the sequence of the 3E10-HC is at least about 98% identical to 3E10-HC-h7m (SEQ ID NO:77). In embodiments, the sequence of the 3E10-HC is at least about 99% identical to 3E10-HC-h7m (SEQ ID NO:77). In embodiments, the sequence of the 3E10-HC is 3E10-HC-h7m (SEQ ID NO:77).

In embodiments, an ADC provided herein comprises a humanized 3E10 antibody, or antigen-binding fragment thereof, comprising a light chain (3E10-LC) comprising an amino acid sequence that is at least about 95% identical to an amino acid sequence selected from the group consisting of 3E10-LC-h1 (SEQ ID NO:97), 3E10-LC-h2 (SEQ ID NO:98), 3E10-LC-h3 (SEQ ID NO:99), 3E10-LC-h4 (SEQ ID NO:100), 3E10-LC-h5 (SEQ ID NO:101), and 3E10-LC-h6 (SEQ ID NO:102) and a heavy chain (3E10-HC) comprising an amino acid sequence that is at least about 95% identical to an amino acid sequence selected from the group consisting of 3E10-HC-h1 (SEQ ID NO:78), 3E10-HC-h2 (SEQ ID NO:79), 3E10-HC-h3 (SEQ ID NO:80), 3E10-HC-h4 (SEQ ID NO:81), 3E10-HC-h5 (SEQ ID NO:82), 3E10-HC-h6 (SEQ ID NO:83), and 3E10-HC-h7 (SEQ ID NO:84).

In embodiments, the sequence of the 3E10-LC is at least about 95% identical to 3E10-LC-h1 (SEQ ID NO:97. In embodiments, the sequence of the 3E10-LC is at least about 96% identical to 3E10-LC-h1 (SEQ ID NO:97). In embodiments, the sequence of the 3E10-LC is at least about 97% identical to 3E10-LC-h1 (SEQ ID NO:97). In embodiments, the sequence of the 3E10-LC is at least about 98% identical to 3E10-LC-h1 (SEQ ID NO:97). In embodiments, the sequence of the 3E10-LC is at least about 99% identical to 3E10-LC-h1 (SEQ ID NO:97). In embodiments, the sequence of the 3E10-LC is 3E10-LC-h1 (SEQ ID NO:97).

In embodiments, the sequence of the 3E10-LC is at least about 95% identical to 3E10-LC-h2 (SEQ ID NO:98). In embodiments, the sequence of the 3E10-LC is at least about 96% identical to 3E10-LC-h2 (SEQ ID NO:98). In embodiments, the sequence of the 3E10-LC is at least about 97% identical to 3E10-LC-h2 (SEQ ID NO:98). In embodiments, the sequence of the 3E10-LC is at least about 98% identical to 3E10-LC-h2 (SEQ ID NO:98). In embodiments, the sequence of the 3E10-LC is at least about 99% identical to 3E10-LC-h2 (SEQ ID NO:98). In embodiments, the sequence of the 3E10-LC is 3E10-LC-h2 (SEQ ID NO:98).

In embodiments, the sequence of the 3E10-LC is at least about 95% identical to 3E10-LC-h3 (SEQ ID NO:99). In embodiments, the sequence of the 3E10-LC is at least about 96% identical to 3E10-LC-h3 (SEQ ID NO:99). In embodiments, the sequence of the 3E10-LC is at least about 97% identical to 3E10-LC-h3 (SEQ ID NO:99). In embodiments, the sequence of the 3E10-LC is at least about 98% identical to 3E10-LC-h3 (SEQ ID NO:99). In embodiments, the sequence of the 3E10-LC is at least about 99% identical to 3E10-LC-h3 (SEQ ID NO:99). In embodiments, the sequence of the 3E10-LC is 3E10-LC-h3 (SEQ ID NO:99).

In embodiments, the sequence of the 3E10-LC is at least about 95% identical to 3E10-LC-h4 (SEQ ID NO:100). In embodiments, the sequence of the 3E10-LC is at least about 96% identical to 3E10-LC-h4 (SEQ ID NO:100). In embodiments, the sequence of the 3E10-LC is at least about 97% identical to 3E10-LC-h4 (SEQ ID NO:100). In embodiments, the sequence of the 3E10-LC is at least about 98% identical to 3E10-LC-h4 (SEQ ID NO:100). In embodiments, the sequence of the 3E10-LC is at least about 99% identical to 3E10-LC-h4 (SEQ ID NO:100). In embodiments, the sequence of the 3E10-LC is 3E10-LC-h4 (SEQ ID NO:100).

In embodiments, the sequence of the 3E10-LC is at least about 95% identical to 3E10-LC-h5 (SEQ ID NO:101). In embodiments, the sequence of the 3E10-LC is at least about 96% identical to 3E10-LC-h5 (SEQ ID NO:101). In embodiments, the sequence of the 3E10-LC is at least about 97% identical to 3E10-LC-h5 (SEQ ID NO:101). In embodiments, the sequence of the 3E10-LC is at least about 98% identical to 3E10-LC-h5 (SEQ ID NO:101). In embodiments, the sequence of the 3E10-LC is at least about 99% identical to 3E10-LC-h5 (SEQ ID NO:101). In embodiments, the sequence of the 3E10-LC is 3E10-LC-h5 (SEQ ID NO:101).

In embodiments, the sequence of the 3E10-LC is at least about 95% identical to 3E10-LC-h6 (SEQ ID NO:102). In embodiments, the sequence of the 3E10-LC is at least about 96% identical to 3E10-LC-h6 (SEQ ID NO:102). In embodiments, the sequence of the 3E10-LC is at least about 97% identical to 3E10-LC-h6 (SEQ ID NO:102). In embodiments, the sequence of the 3E10-LC is at least about 98% identical to 3E10-LC-h6 (SEQ ID NO:102). In embodiments, the sequence of the 3E10-LC is at least about 99% identical to 3E10-LC-h6 (SEQ ID NO:102). In embodiments, the sequence of the 3E10-LC is 3E10-LC-h6 (SEQ ID NO:102).

In embodiments, the sequence of the 3E10-HC is at least about 95% identical to 3E10-HC-h1 (SEQ ID NO:78). In embodiments, the sequence of the 3E10-HC is at least about 96% identical to 3E10-HC-h1 (SEQ ID NO:78). In embodiments, the sequence of the 3E10-HC is at least about 97% identical to 3E10-HC-h1 (SEQ ID NO:78). In embodiments, the sequence of the 3E10-HC is at least about 98% identical to 3E10-HC-h1 (SEQ ID NO:78). In embodiments, the sequence of the 3E10-HC is at least about 99% identical to 3E10-HC-h1 (SEQ ID NO:78). In embodiments, the sequence of the 3E10-HC is 3E10-HC-h1 (SEQ ID NO:78).

In embodiments, the sequence of the 3E10-HC is at least about 95% identical to 3E10-HC-h2 (SEQ ID NO:79). In embodiments, the sequence of the 3E10-HC is at least about 96% identical to 3E10-HC-h2 (SEQ ID NO:79). In embodiments, the sequence of the 3E10-HC is at least about 97% identical to 3E10-HC-h2 (SEQ ID NO:79). In embodiments, the sequence of the 3E10-HC is at least about 98% identical to 3E10-HC-h2 (SEQ ID NO:79). In embodiments, the sequence of the 3E10-HC is at least about 99% identical to 3E10-HC-h2 (SEQ ID NO:79). In embodiments, the sequence of the 3E10-HC is 3E10-HC-h2 (SEQ ID NO:79).

In embodiments, the sequence of the 3E10-HC is at least about 95% identical to 3E10-HC-h3 (SEQ ID NO:80). In embodiments, the sequence of the 3E10-HC is at least about 96% identical to 3E10-HC-h3 (SEQ ID NO:80). In embodiments, the sequence of the 3E10-HC is at least about 97% identical to 3E10-HC-h3 (SEQ ID NO:80). In embodiments, the sequence of the 3E10-HC is at least about 98% identical to 3E10-HC-h3 (SEQ ID NO:80). In embodiments, the sequence of the 3E10-HC is at least about 99% identical to 3E10-HC-h3 (SEQ ID NO:80). In embodiments, the sequence of the 3E10-HC is 3E10-HC-h3 (SEQ ID NO:80).

In embodiments, the sequence of the 3E10-HC is at least about 95% identical to 3E10-HC-h4 (SEQ ID NO:81). In embodiments, the sequence of the 3E10-HC is at least about 96% identical to 3E10-HC-h4 (SEQ ID NO:81). In embodiments, the sequence of the 3E10-HC is at least about 97% identical to 3E10-HC-h4 (SEQ ID NO:81). In embodiments, the sequence of the 3E10-HC is at least about 98% identical to 3E10-HC-h4 (SEQ ID NO:81). In embodiments, the sequence of the 3E10-HC is at least about 99% identical to 3E10-HC-h4 (SEQ ID NO:81). In embodiments, the sequence of the 3E10-HC is 3E10-HC-h4 (SEQ ID NO:81).

In embodiments, the sequence of the 3E10-HC is at least about 95% identical to 3E10-HC-h5 (SEQ ID NO:82). In embodiments, the sequence of the 3E10-HC is at least about 96% identical to 3E10-HC-h5 (SEQ ID NO:82). In embodiments, the sequence of the 3E10-HC is at least about 97% identical to 3E10-HC-h5 (SEQ ID NO:82). In embodiments, the sequence of the 3E10-HC is at least about 98% identical to 3E10-HC-h5 (SEQ ID NO:82). In embodiments, the sequence of the 3E10-HC is at least about 99% identical to 3E10-HC-h5 (SEQ ID NO:82). In embodiments, the sequence of the 3E10-HC is 3E10-HC-h5 (SEQ ID NO:82).

In embodiments, the sequence of the 3E10-HC is at least about 95% identical to 3E10-HC-h6 (SEQ ID NO:83). In embodiments, the sequence of the 3E10-HC is at least about 96% identical to 3E10-HC-h6 (SEQ ID NO:83). In embodiments, the sequence of the 3E10-HC is at least about 97% identical to 3E10-HC-h6 (SEQ ID NO:83). In embodiments, the sequence of the 3E10-HC is at least about 98% identical to 3E10-HC-h6 (SEQ ID NO:83). In embodiments, the sequence of the 3E10-HC is at least about 99% identical to 3E10-HC-h6 (SEQ ID NO:83). In some aspects, the sequence of the 3E10-HC is 3E10-HC-h6 (SEQ ID NO:83).

In embodiments, the sequence of the 3E10-HC is at least about 95% identical to 3E10-HC-h7 (SEQ ID NO:84). In embodiments, the sequence of the 3E10-HC is at least about 96% identical to 3E10-HC-h7 (SEQ ID NO:84). In embodiments, the sequence of the 3E10-HC is at least about 97% identical to 3E10-HC-h7 (SEQ ID NO:84). In embodiments, the sequence of the 3E10-HC is at least about 98% identical to 3E10-HC-h7 (SEQ ID NO:84). In embodiments, the sequence of the 3E10-HC is at least about 99% identical to 3E10-HC-h7 (SEQ ID NO:84). In embodiments, the sequence of the 3E10-HC is 3E10-HC-h7 (SEQ ID NO:84).

In embodiments, an ADC comprising a humanized 3E10 antibody or antigen-binding fragment thereof described herein has CDR sequences corresponding to those in the parent 3E10 antibody, optionally including a D31N amino acid substitution in the VH CDR1. Accordingly, in embodiments, a humanized 3E10 antibody or antigen-binding fragment thereof includes a light chain variable domain (VL) complementarity determining region (CDR) 1 comprising the amino acid sequence of 3E10-VL-CDR1 (SEQ ID NO: 9), a VL CDR2 comprising the amino acid sequence of 3E10-VL-CDR2 (SEQ ID NO: 10), a VL CDR3 comprising the amino acid sequence of 3E10-VL-CDR3 (SEQ ID NO: 11), a heavy chain variable domain (VH) CDR1 comprising the amino acid sequence of 3E10-VH-CDR1a (SEQ ID NO: 16), a VH CDR2 comprising the amino acid sequence of 3E10-VH-CDR2 (SEQ ID NO: 4), and a VH CDR3 comprising the amino acid sequence of 3E10-VH-CDR3 (SEQ ID NO: 5).

In embodiments, an ADC comprising a humanized 3E10 antibody or antigen-binding fragment thereof described herein includes CDR sequences from a variant humanized 3E10 antibody that includes a D31N amino acid substitution in the VH CDR1 (SEQ ID NO:15).

In embodiments, an ADC comprising a humanized 3E10 antibody or antigen-binding fragment thereof described herein includes a set of complementarity determining regions (CDRs) collectively having no more than seven amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising a humanized 3E10 antibody or antigen-binding fragment thereof described herein includes a set of complementarity determining regions (CDRs) collectively having no more than ten amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising a humanized 3E10 antibody or antigen-binding fragment thereof described herein includes a set of complementarity determining regions (CDRs) collectively having no more than nine amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising a humanized 3E10 antibody or antigen-binding fragment thereof described herein includes a set of complementarity determining regions (CDRs) collectively having no more than eight amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising a humanized 3E10 antibody or antigen-binding fragment thereof described herein includes a set of complementarity determining regions (CDRs) collectively having no more than seven amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising a humanized 3E10 antibody or antigen-binding fragment thereof described herein includes a set of complementarity determining regions (CDRs) collectively having no more than six amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising a humanized 3E10 antibody or antigen-binding fragment thereof described herein includes a set of complementarity determining regions (CDRs) collectively having no more than five amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising a humanized 3E10 antibody or antigen-binding fragment thereof described herein includes a set of complementarity determining regions (CDRs) collectively having no more than four amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising a humanized 3E10 antibody or antigen-binding fragment thereof described herein includes a set of complementarity determining regions (CDRs) collectively having no more than three amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising a humanized 3E10 antibody or antigen-binding fragment thereof described herein includes a set of complementarity determining regions (CDRs) collectively having no more than two amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising a humanized 3E10 antibody or antigen-binding fragment thereof described herein includes a set of complementarity determining regions (CDRs) collectively having no more than one amino acid substitution, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

Accordingly, in embodiments, an ADC described herein can comprise a humanized 3E10 antibody or antigen-binding fragment thereof includes a light chain variable domain (VL) complementarity determining region (CDR) 1 comprising the amino acid sequence of 3E10-VL-CDR1 (SEQ ID NO: 9), a VL CDR2 comprising the amino acid sequence of 3E10-VL-CDR2 (SEQ ID NO: 10), a VL CDR3 comprising the amino acid sequence of 3E10-VL-CDR3 (SEQ ID NO: 11), a heavy chain variable domain (VH) CDR1 comprising the amino acid sequence of3E10-VH-CDR1_D31N (SEQ ID NO: 15), a VH CDR2 comprising the amino acid sequence of 3E10-VH-CDR2 (SEQ ID NO: 4), and a VH CDR3 comprising the amino acid sequence of 3E10-VH-CDR3 (SEQ ID NO: 5).

In embodiments, an ADC comprising a humanized 3E10 antibody or antigen-binding fragment thereof described herein includes a set of complementarity determining regions (CDRs) collectively having no more than 12, 11, 10, 9, 8, 7, 6, 5, 4, 3, 2, or 1 amino acid substitutions, relative to the CDR sequences of 3E10-D31N variant (SEQ ID NOs:15-18 and 22-24), selected from, but not limited to, a G to S substitution at position 5 of VH CDR2, a T to S substitution at position 14 of VH CDR2, an S to T substitution at position 5 of VL CDR1, an M to L substitution at position 14 of VL CDR1, an H to A substitution at position 15 of VL CDR1, and an E to Q substitution at position 6 of VL CDR2.

Accordingly, in embodiments, an ADC comprising a humanized 3E10 antibody or antigen-binding fragment thereof includes VH CDR2 comprising the amino acid sequence of 3E10-VH-CDR2.1 (SEQ ID NO: 26) or 3E10-VH-CDR2.2 (SEQ ID NO: 27). In embodiments, the 3E10 antibody or antigen-binding fragment thereof further includes VL CDRs 1-3 (SEQ ID NOs:9-11), and VH CDRs 1 and 3 (SEQ ID NOs:3 and 5) according to the parent 3E10 antibody. In embodiments, the 3E10 antibody or antigen-binding fragment thereof further includes VL CDRs 1-3 (SEQ ID NOs:22-24), and VH CDRs 1 and 3 (SEQ ID NOs:15 and 18) according to the 3E10-D31N variant.

Similarly, in embodiments, an ADC comprising a humanized 3E10 antibody or antigen-binding fragment thereof includes VL CDR1 comprising the amino acid sequence of 3E10-VL-CDR1.1 (SEQ ID NO: 28) or 3E10-VL-CDR1.2 (SEQ ID NO: 29). In embodiments, the 3E10 antibody or variant thereof, or antigen-binding fragment thereof further includes VL CDRs 2 and 3 (SEQ ID NOs:10 and 11), and VH CDRs 1-3 (SEQ ID NOs:3-5) according to the parent 3E10 antibody. In embodiments, the humanized 3E10 antibody or variant thereof, or antigen-binding fragment thereof further includes VL CDRs 2 and 3 (SEQ ID NOs:23 and 24), and VH CRDs 1-3 (SEQ ID NOs:15, 17 and 18) according to the 3E10- D31N variant.

Similarly, in embodiments, an ADC comprising a humanized 3E10 antibody or antigen-binding fragment thereof includes VL CDR2 comprising the amino acid sequence of 3E10-VL-CDR2.1 (SEQ ID NO: 30). In embodiments, the 3E10 antibody or antigen-binding fragment thereof further includes VL CDRs 1 and 3 (SEQ ID NOs:9 and 11), and VH CDRs 1-3 (SEQ ID NOs: 3-5) according to the parent 3E10 antibody. In embodiments, the humanized 3E10 antibody or antigen-binding fragment thereof further includes VL CDRs 1 and 3 (SEQ ID NOs:22 and 24), and VH CDRs 1-3 (SEQ ID NOs:15, 17 and 18)according to the 3E10-D31N variant.

While some of the amino acid substitutions described above are fairly conservative substitutions—e.g., an S to T substitution at position 5 of VL CDR—other substitutions are to amino acids that have vastly different properties—e.g., an M to L substitution at position 14 of VL CDR1, an H to A substitution at position 15 of VL CDR1, and an E to Q substitution at position 6 of VL CDR2. This suggests, without being bound by theory, that at least these positions within the 3E10 CDR framework are tolerant to other amino acid substitutions.

Accordingly, in embodiments, an ADC comprising a humanized 3E10 antibody or antigen-binding fragment thereof includes VH CDR2 comprising the amino acid sequence of 3E10-VH-CDR2.3 (SEQ ID NO: 31). In embodiments, the humanized 3E10 antibody or antigen-binding fragment thereof further includes VL CDRs 1-3 (SEQ ID NOs:9-11), and VH CDRs 1 and 3 (SEQ ID NOs:3 and 5) according to the parent 3E10 antibody. In embodiments, the 3E10 antibody or antigen-binding fragment thereof further includes VL CDRs 1-3 (SEQ ID NOs:22-24), and VH CDRs 1 and 3 (SEQ ID NOs:15 and 18) according to the 3E10- D31N.

Similarly, in embodiments, an ADC comprising a humanized 3E10 antibody or antigen-binding fragment thereof includes VL CDR1 comprising the amino acid sequence of 3E10-VL-CDR1.3 (SEQ ID NO: 32). In embodiments, the humanized 3E10 antibody or antigen-binding fragment thereof further includes VL CDRs 2 and 3 (SEQ ID NOs:10 and 11), and VH CDRs 1-3 (SEQ ID NOs:15, 17 and 18) according to the parent 3E10 antibody. In embodiments, the 3E10 antibody or variant thereof, or antigen-binding fragment thereof further includes VL CDRs 2 and 3 (SEQ ID NOs:23 and 24), and VH CDRs 1-3 (SEQ ID NOs:15, 17 and 18) according to the 3E10- D31N variant.

Similarly, in embodiments, an ADC comprising a humanized 3E10 antibody or antigen-binding fragment thereof, includes VL CDR2 comprising the amino acid sequence of 3E10-VL-CDR2.2 (SEQ ID NO: 33). In embodiments, the humanized 3E10 antibody or antigen-binding fragment thereof further includes VL CDRs 1 and 3 (SEQ ID NOs:9 and 11), and VH CDRs 1-3 (SEQ ID NOs:3-5) according to the parent 3E10 antibody. In embodiments, the 3E10 antibody or variant thereof, or antigen-binding fragment thereof further includes VL CDRs 1 and 3 (SEQ ID NOs:22 and 24), and VH CDRs 1-3 (SEQ ID NOs:15, 17 and 18) according to the 3E10- D31N variant.

Accordingly, in embodiments, an ADC comprising a humanized 3E10 antibody or antigen-binding fragment thereof includes VH CDR1 comprising the amino acid sequence of 3E10-VH-CDR1.c1 (SEQ ID NO: 34), 3E10-VH-CDR1.c2(SEQ ID NO: 35), 3E10-VH-CDR1.c3 (SEQ ID NO: 36), 3E10-VH-CDR1.c4 (SEQ ID NO: 37), or 3E10-VH-CDR1.c5 (SEQ ID NO: 38). In embodiments, the humanized 3E10 antibody or antigen-binding fragment thereof further includes VL CDRs 1-3 (SEQ ID NOs:9-11), and VH CDRs 2 and 3 (SEQ ID NOs:4 and 5) according to the parent 3E10 antibody.

Similarly, in embodiments, an ADC comprising a humanized 3E10 antibody or antigen-binding fragment thereof includes VH CDR2 comprising the amino acid sequence of 3E10-VH-CDR2.c1 (SEQ ID NO: 39), 3E10-VH-CDR2.c2 (SEQ ID NO: 40), or 3E10-VH-CDR2.c3 (SEQ ID NO: 41). In embodiments, the humanized 3E10 antibody or antigen-binding fragment thereof further includes VL CDRs 1-3 (SEQ ID NOs:9-11), and VH CDRs 1 and 3 (SEQ ID NOs:3 and 5) according to the parent 3E10 antibody. In embodiments, the 3E10 antibody or antigen-binding fragment thereof further includes VL CDRs 1-3 (SEQ ID NOs:22-24), and VH CDRs 1 and 3 (SEQ ID NOs:15 and 18) according to the 3E10-D31N variant.

Similarly, in embodiments, an ADC comprising a humanized 3E10 antibody or antigen-binding fragment thereof includes VH CDR3 comprising the amino acid sequence of 3E10-VH-CDR3.c1 (SEQ ID NO: 42), 3E10-VH-CDR3.c2 (SEQ ID NO: 43), or 3E10-VH-CDR3.c3 (SEQ ID NO: 44). In embodiments, the humanized 3E10 antibody or antigen-binding fragment thereof further includes VL CDRs 1-3 (SEQ ID NOs:9-11), and VH CDRs 1 and 2 (SEQ ID NOs:3 and 4) according to the parent 3E10 antibody. In embodiments, the 3E10 antibody or variant thereof, or antigen-binding fragment thereof further includes VL CDRs 1-3 (SEQ ID NOs:22-24), and VH CDRs 1 and 2 (SEQ ID NOs:15 and 17) according to the 3E10-D31N variant.

Similarly, in embodiments, an ADC comprising a humanized 3E10 antibody or antigen-binding fragment thereof includes VL CDR1 comprising the amino acid sequence of 3E10-VL-CDR1.c1 (SEQ ID NO: 45), 3E10-VL-CDR1.c2 (SEQ ID NO: 46), 3E10-VL-CDR1.c3 (SEQ ID NO: 47), 3E10-VL-CDR1.c4 (SEQ ID NO: 48), 3E10-VL-CDR1.c5 (SEQ ID NO: 49), or 3E10-VL-CDR1.c6 (SEQ ID NO: 50). In embodiments, the humanized 3E10 antibody or antigen-binding fragment thereof further includes VL CDRs 2 and 3 (SEQ ID NOs:10 and 11), and VH CDRs 1-3 (SEQ ID NOs:3-5) according to the parent 3E10 antibody. In embodiments, the humanized 3E10 antibody or antigen-binding fragment thereof further includes VL CDRs 2 and 3 (SEQ ID NOs:23 and 24), and VH CRDs 1-3 (SEQ ID NOs:15, 17, 18) according to the 3E10-D31N variant.

Similarly, in embodiments, an ADC comprising a humanized 3E10 antibody or antigen-binding fragment thereof includes VL CDR2 comprising the amino acid sequence of 3E10-VL-CDR2.c1 (SEQ ID NO: 51). In embodiments, the humanized 3E10 antibody or antigen-binding fragment thereof further includes VL CDRs 1 and 3 (SEQ ID NOs:9 and 11), and VH CDRs 1-3 (SEQ ID NOs:3-5) according to the parent 3E10 antibody. In embodiments, the humanized 3E10 antibody or antigen-binding fragment thereof further includes VL CDRs 1 and 3 (SEQ ID NOs:22 and 24), and VH CDRs 1-3 (SEQ ID NOs:15, 17 and 18) according to the 3E10-D31N variant.

Similarly, in some aspects, an ADC comprising a humanized 3E10 antibody or antigen-binding fragment thereof includes VL CDR3 comprising the amino acid sequence of 3E10-VL-CDR3.c1 (SEQ ID NO: 52), 3E10-VL-CDR3.c2 (SEQ ID NO: 53), 3E10-VL-CDR3.c3 (SEQ ID NO: 54), 3E10-VL-CDR3.c4 (SEQ ID NO: 55), 3E10-VL-CDR3.c5 (SEQ ID NO: 56), or 3E10-VL-CDR3.c6 (SEQ ID NO: 57). In embodiments, the humanized 3E10 antibody or antigen-binding fragment thereof further includes VL CDRs 1 and 2 (SEQ ID NOs:9 and 10), and VH CDRs 1-3 (SEQ ID NOs:3-5) according to the parent 3E10 antibody. In embodiments, the humanized 3E10 antibody or antigen-binding fragment thereof further includes VL CDRs 1 and 2 (SEQ ID NOs:22 and 23), and VH CDRs 1-3 (SEQ ID NOs:15, 17 and 18) according to the 3E10-D31N variant.

It is also contemplated that an ADC comprising a humanized 3E10 antibody or antigen-binding fragment thereof, as described herein, includes no more than 12, 11, 10, 9, 8, 7, 6, 5, 4, 3, 2, or 1 CDR amino acid substitutions of the CDR amino acid substitutions described above. Further examples of 3E10 variant CDR sequences are described herein (SEQ ID NOs:58-63).

Accordingly, in embodiments, an ADC comprising a humanized 3E10 antibody or antigen-binding fragment thereof includes VH CDR1 comprising the amino acid sequence of 3E10-VH-CDR1m (SEQ ID NO: 58). In embodiments, the humanized 3E10 antibody or antigen-binding fragment thereof further includes VL CDRs 1-3 (SEQ ID NOs:9-11), and VH CDRs 2 and 3 (SEQ ID NOs:4 and 5) according to the parent 3E10 antibody.

Similarly, in embodiments, an ADC comprising a humanized 3E10 antibody or antigen-binding fragment thereof includes VH CDR2 comprising the amino acid sequence of 3E10-VH-CDR2m (SEQ ID NO: 59). In embodiments, the humanized 3E10 antibody or antigen-binding fragment thereof further includes VL CDRs 1-3 (SEQ ID NOs:9-11), and VH CDRs 1 and 3 (SEQ ID NOs: 3 and 5) according to the parent 3E10 antibody. In embodiments, the humanized 3E10 antibody or antigen-binding fragment thereof further includes VL CDRs 1-3 (SEQ ID NOs:22-24), and VH CDRs 1 and 3 (SEQ ID NOs:15 and 18) according to the 3E10-D31N variant.

Similarly, in embodiments, an ADC comprising a humanized 3E10 antibody or antigen-binding fragment thereof includes VH CDR3 comprising the amino acid sequence of 3E10-VH-CDR3m (SEQ ID NO: 60). In embodiments, the humanized 3E10 antibody or antigen-binding fragment thereof further includes VL CDRs 1-3 (SEQ ID NOs:9-11), and VH CDRs 1 and 2 (SEQ ID NOs:3 and 4) according to the parent 3E10 antibody. In embodiments, the humanized 3E10 antibody or antigen-binding fragment thereof further includes VL CDRs 1-3 (SEQ ID NOs:22-24), and VH CDRs 1 and 2(SEQ ID NOs:15 and 17) according to the 3E10-D31N variant.

Similarly, in embodiments, an ADC comprising a humanized 3E10 antibody or antigen-binding fragment thereof includes VL CDR1 comprising the amino acid sequence of 3E10-VL-CDR1m (SEQ ID NO: 61). In embodiments, the humanized 3E10 antibody or antigen-binding fragment thereof further includes VL CDRs 2 and 3 (SEQ ID NOs:10 and 11), and VH CDRs 1-3 (SEQ ID NOs:3-5) according to the parent 3E10 antibody. In embodiments, the humanized 3E10 antibody or antigen-binding fragment thereof further includes VL CDRs 2 and 3 (SEQ ID NOs:23 and 24), and VH CDRs 1-3 (SEQ ID NOs:15, 17, and 18) according to the 3E10-D31N variant.

Similarly, in embodiments, an ADC comprising a humanized 3E10 antibody or antigen-binding fragment thereof includes VL CDR2 comprising the amino acid sequence of 3E10-VL-CDR2m (SEQ ID NO: 62). In embodiments, the humanized 3E10 antibody or antigen-binding fragment thereof further includes VL CDRs 1 and 3 (SEQ ID NOs:9 and 11), and VH CDRs 1-3 (SEQ ID NOs:3-5) according to the parent 3E10 antibody. In embodiments, the humanized 3E10 antibody or antigen-binding fragment thereof further includes VL CDRs 1 and 3 (SEQ ID NOs:22 and 24), and VH CDRs 1-3 (SEQ ID NOs:15, 17 and 18) according to the 3E10-D31N variant.

Similarly, in embodiments, an ADC comprising a humanized 3E10 antibody or antigen-binding fragment thereof includes VL CDR3 comprising the amino acid sequence of 3E10-VL-CDR3m (SEQ ID NO: 63). In embodiments, the humanized 3E10 antibody or antigen-binding fragment thereof further includes VL CDRs 1 and 2 (SEQ ID NOs:9 and 10), and VH CDRs 1-3 (SEQ ID NOs:3-5) according to the parent 3E10 antibody. In embodiments, the humanized 3E10 antibody or antigen-binding fragment thereof further includes VL CDRs 1 and 2 (SEQ ID NOs:9 and 10), and VH CDRs 1-3 (SEQ ID NOs: 15, 17 and 18) according to the 3E10-D31N variant.

In embodiments, an ADC comprising a humanized 3E10 antibody or antigen-binding fragment thereof described herein includes a light chain variable domain (3E10-VL) comprising an amino acid sequence that is at least about 90% identical to an amino acid sequence selected from the group consisting of 3E10-VL-h1 (SEQ ID NO:85), 3E10-VL-h2(SEQ ID NO:86), 3E10-VL-h3 (SEQ ID NO:87), 3E10-VL-h4(SEQ ID NO:88), 3E10-VL-h5 (SEQ ID NO:89), and 3E10-VL-h6 (SEQ ID NO:90), where the light chain variable domain (3E10-VL) further comprises one or more amino acid residues selected from proline (Pro) at position 15, threonine (Thr) at position 22, tyrosine (Tyr) at position 49, Thr at position 74, asparagine (Asn) at position 76, alanine (Ala) at position 80, Asn at position 81, Thr at position 83, Asn at position 85, and valine (Val) at position 104, of the 3E10-VL according to Kabat numbering, and a set of 3E10-VL CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), and where the antibody includes a set of 3E10-VL CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In embodiments, an ADC comprising the humanized 3E10 antibody or antigen-binding fragment thereof includes a set of 3E10-VL CDRs comprising no more than 5 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In embodiments, an ADC comprising the humanized 3E10 antibody or antigen-binding fragment thereof includes a set of 3E10-VL CDRs comprising no more than 4 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In embodiments, an ADC comprising the humanized 3E10 antibody or antigen-binding fragment thereof includes a set of 3E10-VL CDRs comprising no more than 3 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In embodiments, an ADC comprising the humanized 3E10 antibody or antigen-binding fragment thereof includes a set of 3E10-VL CDRs comprising no more than 2 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In embodiments, an ADC comprising the humanized 3E10 antibody or antigen-binding fragment thereof includes a set of 3E10-VL CDRs comprising no more than 1 amino acid substitution relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In embodiments, an ADC comprising the humanized 3E10 antibody or antigen-binding fragment thereof includes a set of 3E10-VL CDRs comprising a set of CDRs having the amino acid sequences of 3E10-VL-CDR1 (SEQ ID NO:9), 3E10-VL-CDR2(SEQ ID NO:10), 3E10-VL-CDR3 (SEQ ID NO:11).

In one aspect, the present disclosure provides an ADC comprising a humanized antibody or antigen-binding fragment thereof with a lysine (Lys) residue at position 49 of the 3E10-VL according to Kabat numbering.

In one aspect, the present disclosure provides an ADC comprising a humanized antibody or antigen-binding fragment thereof with a glutamic acid (Glu) residue at position 81 of the 3E10-VL according to Kabat numbering.

In one aspect, the present disclosure provides an ADC comprising a humanized antibody or antigen-binding fragment thereof with a proline (Pro) residue at position 15 of the 3E10-VL according to Kabat numbering.

In one aspect, the present disclosure provides an ADC comprising a humanized antibody or antigen-binding fragment thereof with a valine (Val) residue at position 104, of the 3E10-VL according to Kabat numbering.

In embodiments, an ADC comprising a humanized 3E10 antibody or antigen-binding fragment thereof described herein includes a heavy chain variable domain (3E10-VH) comprising an amino acid sequence that is at least about 90% identical to an amino acid sequence selected from the group consisting of 3E10-VH-h1 (SEQ ID NO:64), 3E10-VH-h2(SEQ ID NO:65), 3E10-VH-h3 (SEQ ID NO:66), 3E10-VH-h4 (SEQ ID NO:67), 3E10-VH-h5 (SEQ ID NO:68), 3E10-VH-h6 (SEQ ID NO:69), and 3E10-VH-h7 (SEQ ID NO:70), where the heavy chain variable domain (3E10-VH) further comprises one or more amino acid residues selected from glutamine (Gin) at position 13, leucine (Leu) at position 18, arginine (Arg) at position 19, glycine (Gly) at position 42, serine (Ser) at position 49, Ser at position 77, tyrosine (Tyr) at position 79, Asn at position 82, Ala at position 84, Val at position 89, leucine (Leu) at position 108, Val at position 109, and Ser at position 113, of the 3E10-VH according to Kabat numbering, and where the antibody includes a set of 3E10-VH CDRs collectively having no more than 6 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising the humanized 3E10 antibody or antigen-binding fragment thereof includes a set of 3E10-VH CDRs comprising no more than 5 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising the humanized 3E10 antibody or antigen-binding fragment thereof includes a set of 3E10-VH CDRs comprising no more than 4 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising the humanized 3E10 antibody or antigen-binding fragment thereof includes a set of 3E10-VH CDRs comprising no more than 3 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising the humanized 3E10 antibody or antigen-binding fragment thereof includes set of 3E10-VH CDRs comprising no more than 2 amino acid substitutions relative to the a set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising the humanized 3E10 antibody or antigen-binding fragment thereof includes a set of 3E10-VH CDRs comprising no more than 1 amino acid substitution relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5).

In embodiments, an ADC comprising the humanized 3E10 antibody or antigen-binding fragment thereof includes a set of 3E10-VH CDRs comprising no more than 5, 4, 3, 2, or 1 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5).

In one aspect, the present disclosure provides an ADC comprising a humanized antibody or antigen-binding fragment thereof with an arginine (Arg) residue at position 18 of the 3E10-VH according to Kabat numbering.

In one aspect, the present disclosure provides an ADC comprising a humanized antibody or antigen-binding fragment thereof with a (Lys) residue at position 19 of the 3E10-VH according to Kabat numbering.

In one aspect, the present disclosure provides an ADC comprising a humanized antibody or antigen-binding fragment thereof with an alanine (Ala) residue at position 49 of the 3E10-VH according to Kabat numbering.

In one aspect, the present disclosure provides an ADC comprising a humanized antibody or antigen-binding fragment thereof with a glutamine (Gln) residue at position 13, of the 3E10-VH according to Kabat numbering.

In one aspect, the present disclosure provides an ADC comprising a humanized antibody or antigen-binding fragment thereof with a leucine (Leu) residue at position 108, of the 3E10-VH according to the Kabat numbering.

In one aspect, the present disclosure provides an ADC comprising a humanized antibody or antigen-binding fragment thereof with a valine (Val) residue at position 109, of the 3E10-VH according to Kabat numbering.

In one aspect, the present disclosure provides an ADC comprising a humanized antibody or antigen-binding fragment thereof with a serine (Ser) residue at position 113, of the 3E10-VH according to Kabat numbering.

In embodiments, the present disclosure provides an ADC comprising a humanized 3E10 antibody or antigen-binding fragment thereof with a fragment crystallizable (Fc) region.

In embodiments, the present disclosure provides an ADC comprising a humanized 3E10 antibody or antigen-binding fragment thereof with an Fc region selected from a human IgG1 Fc, a human IgG2a Fc, a human IgG2b Fc, a human IgG3 Fc, and a human IgG4 Fc.

In embodiments, the present disclosure provides an ADC comprising humanized 3E10 antibodies or variants thereof, or antigen-binding fragments thereof comprising a heavy chain constant domain (CH).

In embodiments, a ADC comprising the humanized 3E10 antibody or variant thereof, or antigen-binding fragment thereof comprises an Fc region selected from a human γ1 CH1, a human γ2 CH1, a human γ3 CH1, and a human γ4 CH1.

In embodiments, the present disclosure provides an ADC comprising a humanized 3E10 antibody or variant thereof, or antigen-binding fragment thereof comprising a light chain constant domain (CL).

In one aspect, the present disclosure provides an ADC comprising a humanized 3E10 antibody or variant comprising an Fc region selected from the group consisting of a human λ CL and a human κ CL.

In embodiments, an ADC comprising the humanized 3E10 antibody or antigen-binding fragment thereof comprising a combination of a light chain variable domain (VL) and a heavy chain variable domain (VH) selected from 3E10-VL-h1 (SEQ ID NO:85) and 3E10-VH-h1 (SEQ ID NO:64), 3E10-VL-h1 (SEQ ID NO:85) and 3E10-VH-h2 (SEQ ID NO:65), 3E10-VL-h1 (SEQ ID NO:85) and 3E10-VH-h3 (SEQ ID NO:66), 3E10-VL-h1 (SEQ ID NO:85) and 3E10-VH-h4 (SEQ ID NO:67), 3E10-VL-h2 (SEQ ID NO:86) and 3E10-VH-h1 (SEQ ID NO:64), 3E10-VL-h2 (SEQ ID NO:86) and 3E10-VH-h2 (SEQ ID NO:65), 3E10-VL-h3 (SEQ ID NO:87) and 3E10-VH-h1 (SEQ ID NO:64), 3E10-VL-h5 (SEQ ID NO:89) and 3E10-VH-h5 (SEQ ID NO:68), 3E10-VL-h5 (SEQ ID NO:89) and 3E10-VH-h6 (SEQ ID NO:69), 3E10-VL-h6 (SEQ ID NO:90) and 3E10-VH-h5 (SEQ ID NO:68), and 3E10-VL-h6 (SEQ ID NO:90) and 3E10-VH-h6 (SEQ ID NO:69).

In embodiments, an ADC comprising the humanized 3E10 antibody or antigen-binding fragment thereof comprises a combination of a light chain variable domain (VL) of 3E10-VL-h6 (SEQ ID NO:90) and a heavy chain variable domain (VH) of 3E10-VH-h6 (SEQ ID NO:69).

Antibodies useful in the compositions, conjugates, and methods described herein include whole immunoglobulin (i.e., an intact antibody) of any class, fragments thereof, and synthetic proteins containing at least the antigen-binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies. Therefore, the antibodies typically contain at least the CDRs necessary to maintain DNA binding and/or interfere with DNA repair.

The 3E10 antibody is typically a monoclonal 3E10, or a variant, derivative, fragment, fusion, or humanized form thereof that binds the same or different epitope(s) as 3E10.

A deposit according to the terms of the Budapest Treaty of a hybridoma cell line producing monoclonal antibody 3E10 was received on Sep. 6, 2000, and accepted by, American Type Culture Collection (ATCC), 10801 University Blvd., Manassas, VA 20110-2209, USA, and given Patent Deposit Number PTA-2439. Thus, the antibody can have the same or different epitope specificity as monoclonal antibody 3E10 produced by ATCC No. PTA 2439 hybridoma. The antibody can have the paratope of monoclonal antibody 3E10. The antibody can be a single chain variable fragment of 3E10, or a variant, e.g., a conservative variant thereof. For example, the antibody can be a single chain variable fragment of 3E10 (3E10 Fv), or a variant thereof.

Additionally, or alternatively, the heavy chain complementarity determining regions (CDRs) can be defined according to the IMGT system. The complementarity determining regions (CDRs) as identified by the IMGT system include CDR H1.3 (original sequence): GFTFSDYG (SEQ ID NO:117); CDR H1.4 (with D31N mutation): GFTFSNYG (SEQ ID NO:118); CDR H2.2: ISSGSSTI (SEQ ID NO:119) and variant ISSSSSTI (SEQ ID NO:120); CDR H3.2: ARRGLLLDY (SEQ ID NO:121).

Additionally, or alternatively, the light chain complementarity determining regions (CDRs) can be defined according to the IMGT system. The complementarity determining regions (CDRs) as identified by the IMGT system include CDR L1.2 KSVSTSSYSY (SEQ ID NO:122) and variant KTVSTSSYSY (SEQ ID NO:123); CDR L2.2: YAS (SEQ ID NO:124); CDR L3.2: QHSREFPWT (SEQ ID NO:125).

The disclosed compositions, conjugates, and methods typically utilize antibodies that maintain the ability to penetrate cells, and optionally nuclei. The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur J Immunol 38, 3178-3190 (2008), Yanase et al., J Clin Invest 100, 25-31 (1997)). The 3E10 antibodies and antigen-binding fragments or variants thereof can transit cellular membranes via an equilibrative nucleoside (ENT) transporter. In embodiments, 3E10 transits cellular membranes via an ENT1, ENT2, ENT3 or ENT4 transporter (See, e.g., WO 2015/106290 A1 and WO 2016/033324 A1, each of which is incorporated by reference herein, in its entirety). In embodiments, 3E10 penetrates cells in an Fc-independent mechanism (as evidenced by the ability of 3E10 fragments lacking an Fc to penetrate cells) but involves presence of the nucleoside transporter ENT2 (Weisbart et al., Sci Rep 5:12022. doi: 10.1038/srep12022. (2015), Zack et al., J Immunol 157, 2082-2088 (1996), Hansen et al., J Biol Chem 282, 20790-20793 (2007)). Thus, in embodiments, the antibodies utilized in the disclosed compositions, conjugates, and methods are ones that penetrates cells in an Fc-independent mechanism but involves presence of the nucleoside transporter ENT2.

Mutations in 3E10 that interfere with its ability to bind DNA can render the antibody incapable of nuclear penetration. Thus, typically the disclosed variants and humanized forms of the antibody maintain the ability to bind nucleic acids, particularly DNA. In addition, 3E10 scFv has previously been shown capable of penetrating into living cells and nuclei in an ENT2-dependent manner, with efficiency of uptake impaired in ENT2-deficient cells (Hansen, et al., J. Biol. Chem. 282, 20790-20793 (2007)). Thus, in embodiments, the disclosed variants and humanized forms of the antibody maintain the ability penetrate into cell nuclei in an ENT2-dependent manner.

### B. Therapeutic Agents

As used herein, the terms “therapeutic agent,” “payload moiety,” “payload,” “drug moiety,” “drug,” “anti-tumor drug,” “biologically active molecule,” “active molecule,” and “chemotherapeutic warhead” all refer to a molecule that has a biological, a cytotoxic, or a therapeutic effect in a cell. In embodiments, the drug or active molecule can be an inorganic molecule, an organic molecule, a small organic molecule, a drug compound, a peptide, or a polypeptide. In embodiments, the drug or active molecule can be a functional nucleic acid, such as an oligonucleotide or a polynucleotide.

### 1. Small Molecules

In embodiments, the therapeutic agent is an anti-tumor drug or active molecule. In embodiments, the anti-tumor drug or active molecule can be a cytotoxic agent. The term “cytotoxic agent” refers generally to substances that destroy cells. In embodiments, the anti-tumor drug or active molecule can be a DNA damage inducing agent, a DNA repair inhibitor, an immune modulatory molecule, an alkylating agent, a microtubule inhibitor, an immune checkpoint inhibitor, an angiogenesis inhibitor, adoptive cell therapy, or a topoisomerase inhibitor, or any other cytotoxic agent or chemotherapeutic small molecule known in the art.

In embodiments of an ADC provided herein, P is a DNA damage inducing agent, a DNA repair inhibitor, an immune modulatory molecule, an alkylating agent, a microtubule inhibitor, an immune checkpoint inhibitor, an angiogenesis inhibitor, adoptive cell therapy, or a topoisomerase inhibitor.

In embodiments, the anti-tumor drug or active molecule can be selected from maytansinoids, benzodiazepines, auristatins, tecans, taxoids, CC-1065, (4S)-4,11-Diethyl-4,9-dihydroxy-1,4-dihydro-3H,14H-pyrano[3′,4′:6,7]indolizino[1,2-b]quinoline-3,14-dione (SN38), exatecan, monomethyl auristatin E (MMAE), monomethyl auristatin F (MMAF), pyrrolobenzodiazepines (PBDs), PROteolysis TArgeting Chimera (PROTAC), deruxtecan (Dxd), calicheamicins, duocarmycins, stimulator of interferon genes (STING) agonists, PNU-159682, NMS249, IMGN Camp 1, duocarmycin hydroxybenzamide azaindole (DUBA) and a prodrug anti-tumor drug thereof. In embodiments, the anti-tumor drug can be Pseudomonas aeruginosa exotoxin PE38, calicheaamicin, diphtheria toxin, irinotecans, duocarmycin, exatecan, Staphylococcus aureus enterotoxin A/E-120, doxorubicin, tubulysin, antibacterial antibiotic, shigatoxin, ricin, or urease. In embodiments, the anti-tumor drug can be N(2′)-deacetyl-N(2′)-(3-mercapto-1-oxopropyl)-maytansine (“DM1”). In embodiments, the anti-tumor drug can be N2′-deacetyl-N2′-(4-mercapto-4-methyl-1-oxopentyl) maytansine (“DM4”). In embodiments, the anti-tumor drug can be SN38. In embodiments, the anti-tumor drug can be PNU-159682, NMS249. In embodiments, the anti-tumor drug can be NMS249.

In embodiments, the anti-tumor drug or active molecule can be doxorubicin (ADRIAMYCIN®), cisplatin, carboplatin, bleomycin sulfate, carmustine, chlorambucil (LEUKERAN®), cyclophosphamide (CYTOXAN®; NEOSAR®), lenalidomide (REVLIMID®), bortezomib (VELCADE®), dexamethasone, mitoxantrone, etoposide, cytarabine, bendamustine (TREANDA®), rituximab (RITUXAN®), ifosfamide, vincristine (ONCOVIN®), fludarabine (FLUDARA®), thalidomide (THALOMID®), alemtuzumab (CAMPATH®, ofatumumab (ARZERRA®), everolimus (AFINITOR®, ZORTRESS®), carfilzomib (KYPROLIS™), a proteasome inhibitor, thalidomide analogues that are immunomodulatory drugs (IMiDs), Bet inhibitors or any other cytotoxic agent or chemotherapeutic agents known in the art.

In some embodiments, the drug moiety is selected from any of the following:

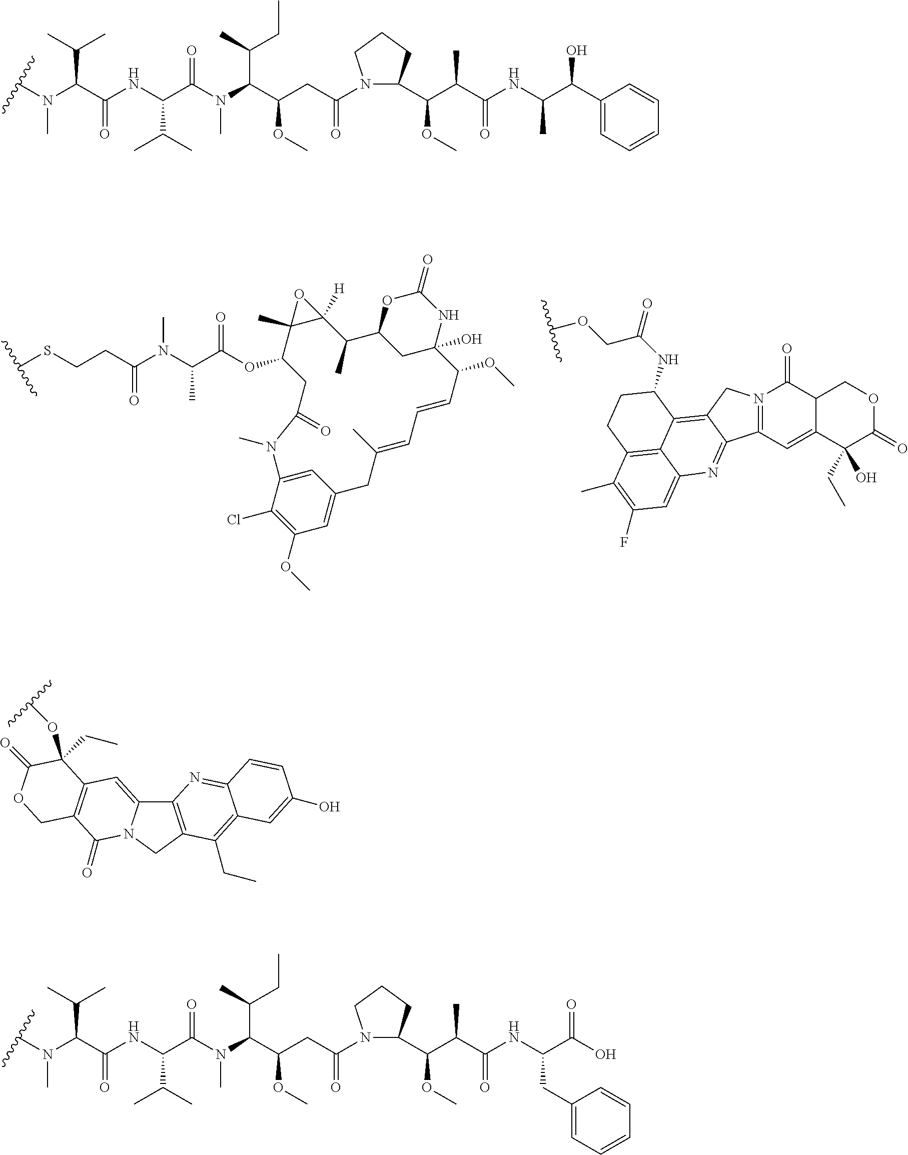

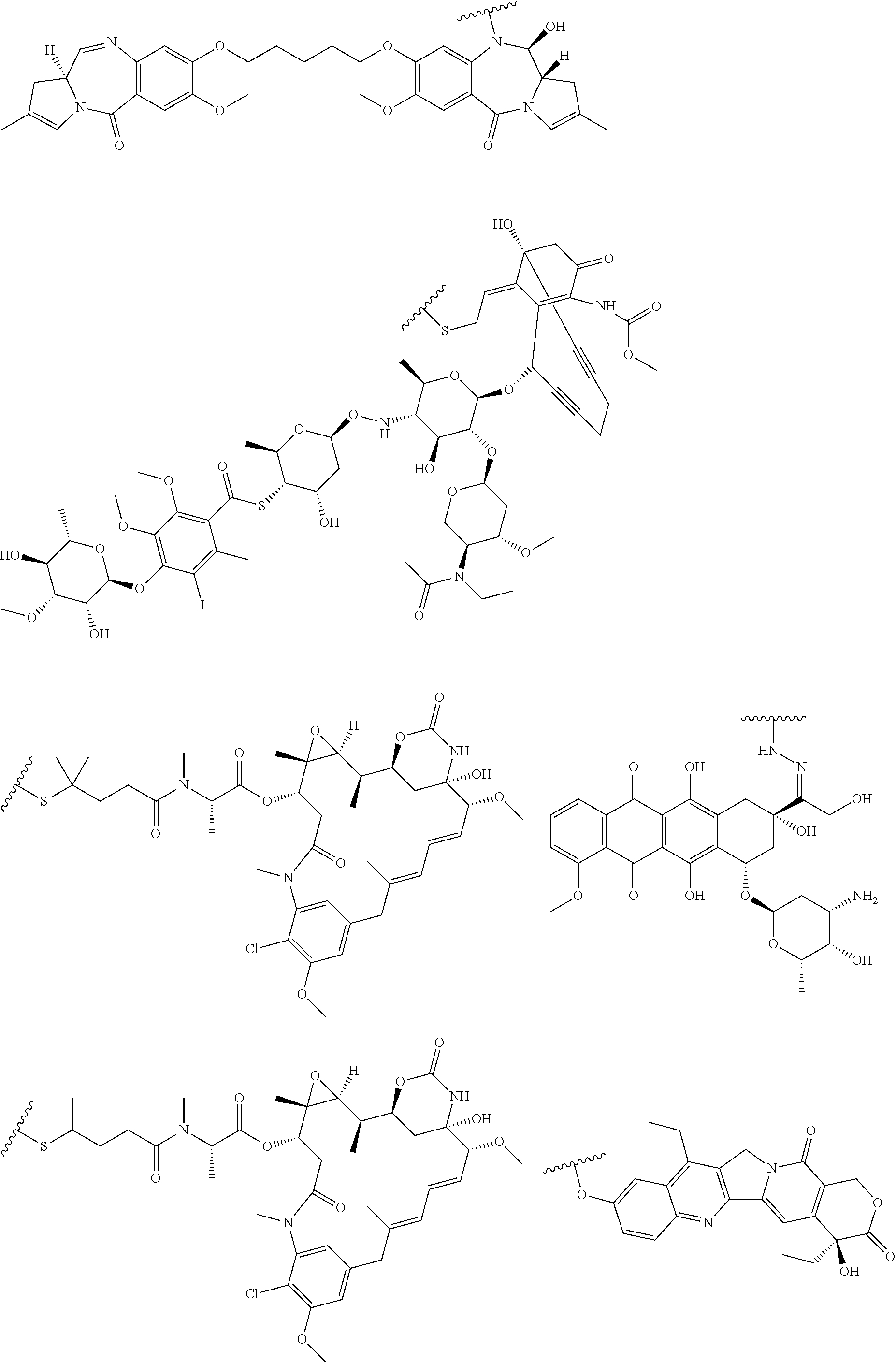

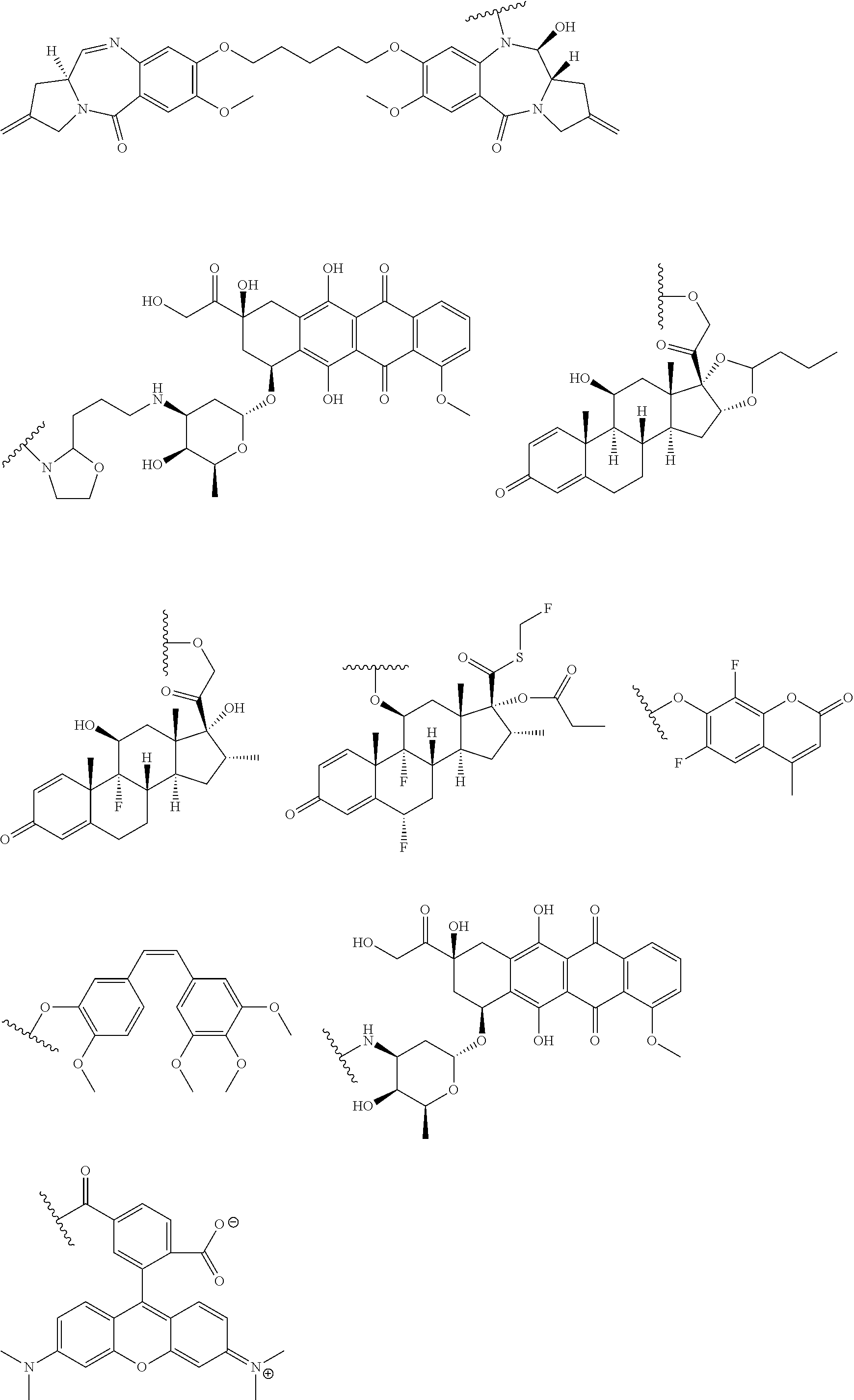

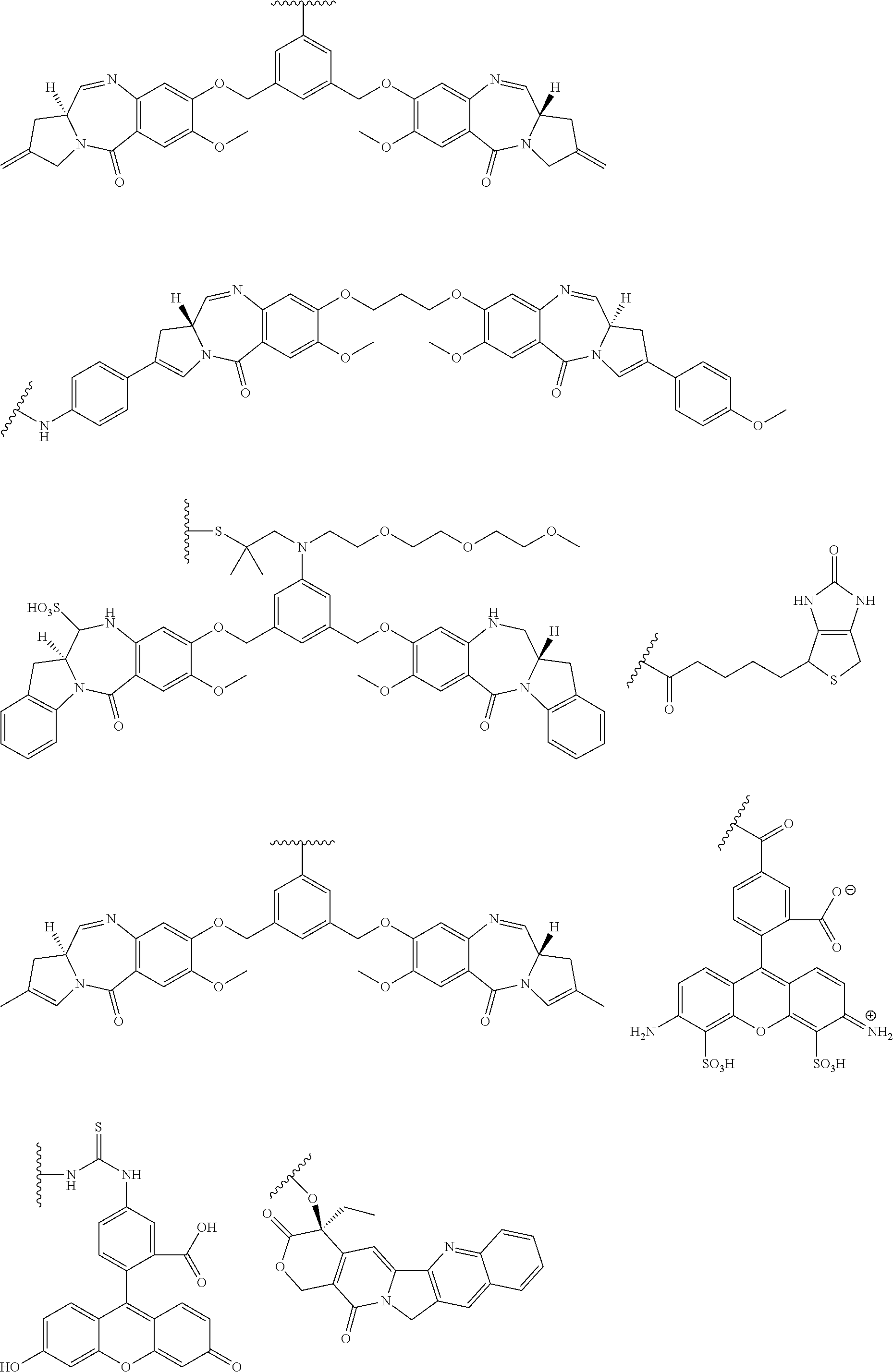

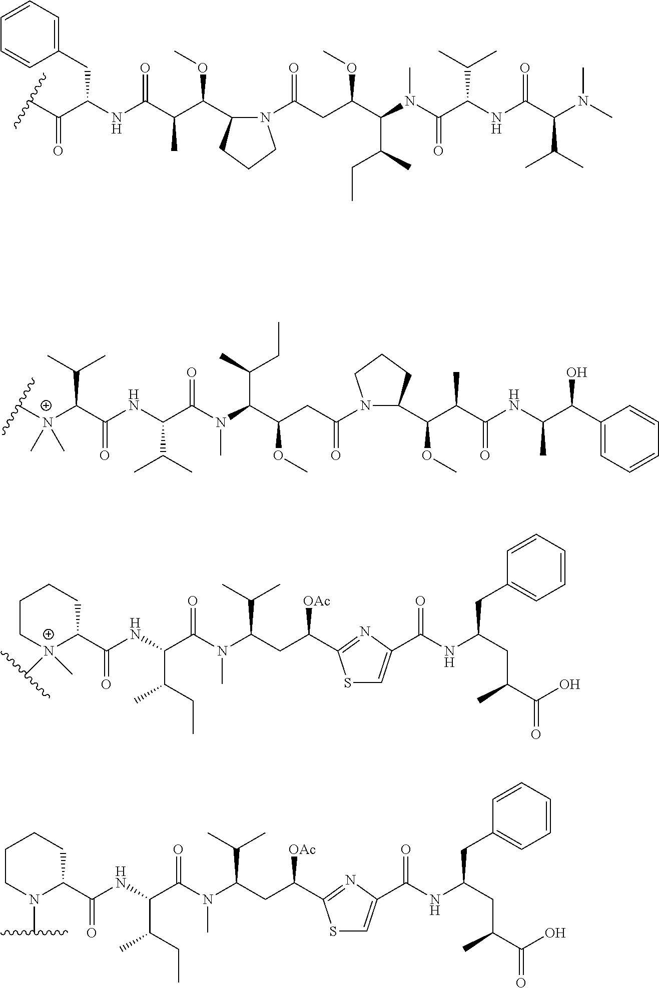

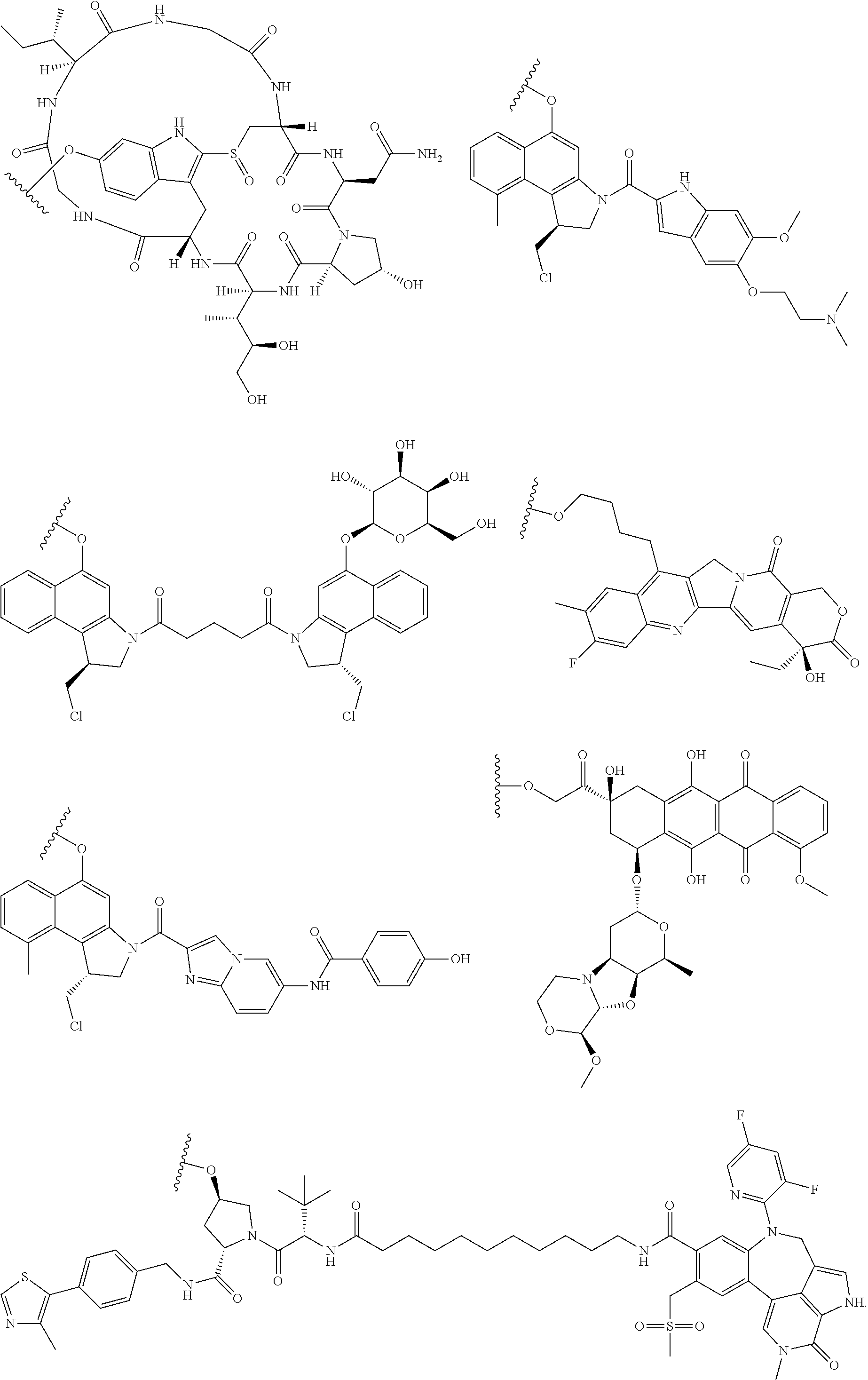

In embodiments of an ADC provided herein, P is a maytansinoid. In embodiments, P is N(2′)-deacetyl-N(2′)-(3-mercapto-1-oxopropyl)-maytansine (DM1). In embodiments, P is N2′-deacetyl-N2′-(4-mercapto-4-methyl-1-oxopentyl) maytansine (DM4).

In embodiments of an ADC provided herein, P is (4S)-4,11-Diethyl-4,9-dihydroxy-1,4-dihydro-3H,14H-pyrano[3′,4′:6,7]indolizino[1,2-b]quinoline-3,14-dione (SN38).

In embodiments of an ADC provided herein, A is a 3E10 antibody or antigen-binding fragment or variant thereof, L is SMCC, and P is DM1.

In embodiments of an ADC provided herein, A is a 3E10 antibody or antigen-binding fragment or variant thereof, L is O-Succinyl, and P is SN38. Therefore, in embodiments L is a cleavable linker.

In embodiments of an ADC provided herein A is a 3E10 antibody or antigen-binding fragment or variant thereof, L is CL2A and P is SN38.

In embodiments of an ADC provided herein A is a 3E10 antibody or antigen-binding fragment or variant thereof, L is MC-vc-PAB and P is PNU-159682.

In embodiments of an ADC provided herein A is a 3E10 antibody or antigen-binding fragment or variant thereof, L is MC-vc-PAB and P is NMS249.

In embodiments, the present disclosure provides an antibody-drug conjugate (ADC) having the formula A-(L-Pr)q, wherein: A is a humanized 3E10 antibody or antigen-binding fragment thereof comprising: (a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO:10), and (c) a VL CDR3 comprising the amino acid sequence of QHSREFPWT (SEQ ID NO: 11), and (d) a heavy chain variable region (VH) CDR1 comprising the amino acid sequence of NYGMH (SEQ ID NO: 15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5), L is O-Succinyl, P is SN38, r is an integer from 1 to 4, and q is an integer from 1 to 16.

In embodiments, ADCs disclosed herein have the formula A-(L-Pr)q, wherein: A is a 3E10 antibody or antigen-binding fragment or variant thereof, L is a linker, P is a payload as described herein, r is an integer from 1 to 4, and q is an integer from 1 to 16; wherein the 3E10 antibody or antigen-binding fragment thereof comprises a heavy chain variable region (VH) CDR1 comprising the amino acid sequence of XYGMH (SEQ ID NO: 16), wherein X=D, N, R, L, or K and corresponds to an amino acid residue at position 31 of the 3E10 heavy chain, and wherein D31 is wild-type 3E10 CDR1.

### C. Linkers

In embodiments, the universal antibody-drug conjugate (ADC) provided herein comprises a linker. The linker (L) described herein can be used to link or conjugate the 3E10 antibody or antigen-binding fragment or variant thereof to an oligonucleotide, a drug, or anti-tumor drug, or any biologically active molecule considered a toxic agent, chemotherapeutic agent, or warhead for use in killing cancer cells. The term “linker” as used herein includes, without limitation, any known linker for use antibody-drug-conjugates known in the art. In embodiments, an ADC conjugate comprises, one, two, three, four, or more linkers.

### 1. Conjugation Sites and Methods

In embodiments, one or more amino acids suitable conjugation of a 3E10 antibody or cell-penetrating fragment thereof provided herein are selected from lysine, cysteine, histidine, arginine, aspartic acid, glutamine, serine, threonine, and tyrosine. In embodiments, one or more amino acids suitable for conjugation are introduced by substitution of one or more amino acids in the 3E10 antibody or cell-penetrating fragment thereof. In embodiments, the one or more conjugated amino acids are lysine or arginine, and conjugation is conducted via amine conjugation. In embodiments, one or more conjugated amino acids are glutamine (Gln) and conjugation is conducted via transglutaminase (TGase) mediated enzymatic conjugation. In some embodiments, one or more conjugated amino acids are cysteine (Cys) and conjugation is conducted via thiol conjugation. For example, NHS-PEG reagent can be used to modify primary amines in a 3E10 antibody or cell-penetrating fragment thereof provided herein.

In embodiments, a method of site-specific conjugation is by means of transglutaminase. Transglutaminases (TGases) which also include bacterial transglutaminase (BTG) are a family of enzymes which catalyze the formation of a covalent bond between the 7-carbonyl-amide group of glutamines and the primary amine group of lysines.

A peptide or antibody can be a substrate for transglutaminase according to the methods of the present disclosure. Thus, in some embodiments, the peptide or antibody contains a Gln or a Lys residue, and in particular a Gln residue. In some embodiments, the peptide or antibody is not a transglutaminase substrate, soone or more Gln or Lys residues, and in particular Gln residues, are inserted into the peptide or antibody sequence to make the peptide or antibody a substrate for transglutaminase. In embodiments, a Gln or Lys residue can be inserted at any position in the peptide or antibody sequence, however, it is preferably inserted at a position where the physiological properties, such as the therapeutic activity of the peptide, is not affected to a degree where the peptide is not useful anymore, e.g., in a therapeutic intervention. Insertions of amino acid residues in peptides can be brought about by standard techniques known to persons skilled in the art, such as post-translational chemical modification or transgenic techniques, as described in U.S. Pat. No. 11,123,439 and US/2016/0355859, the contents of which are hereby incorporated by reference.

Since such transglutaminases also accept substrates other than lysine as amine donor, they are used to modify proteins including antibodies at suitable acceptor glutamines (Josten et al., J. Immunol. Methods 240, 47-54 (2000); Mindt et al., Bioconjugate Chem. 19, 271-278 (2008); Dennler et al., in Antibody Drug Conjugates (Ducry, L., Ed.), pp 205-215, Humana Press. (2013), the contents of which are incorporated herein by reference). Transglutaminases have been used for the conjugation of drugs to antibodies containing artificial glutamine tags which are acceptor glutamine residues which have been introduced into the antibody by genetic engineering (Strop et al., Chem. Biol. 20, 161-167 (2013)). Furthermore, the conserved glutamine residue Q295 (Kabat EU numbering) of the constant region of the heavy chain of antibodies is the only γ-carbonyl-amide donor for the bacterial transglutaminase (EC 2.3.2.13) in the backbone of aglycosylated IgG1 molecules, and is thus an acceptor glutamine, whereas no acceptor glutamine is present in the backbone of IgG1 when the antibody has been glycosylated at position N297 (Kabat EU numbering) of the heavy chain. In summary, bacterial transglutaminase can be used for the conjugation of an amine-donor substrate, for example a drug-linker construct, at an acceptor glutamine residue of an antibody. Such acceptor glutamines can be introduced by engineering of the antibody by mutations or by the generation of aglycosylated antibodies. Such aglycosylated antibodies can be introduced by deglycosylation using N-glycosidase F (PNGase F) or by mutation of N297 of the glycosylation site of the heavy chain (Kabat EU numbering) to any other amino acid except N. The enzymatic conjugation of such aglycosylated antibodies using bacterial transglutaminase has been described for aglycosylated antibody variants containing the mutations N297D, N297Q or N297S (see U.S. Pat. Nos. 9,764,038 and 9,764,038, the contents of which are incorporated by reference). The enzymatic conjugation of such aglycosylated antibodies by means of transglutaminase generally affords AOCs having a DAR of 2, in which both heavy chains are specifically functionalized at position Q295 (Kabat EU numbering). Only mutation N297Q of the heavy chain affords an additional conjugation site per heavy chain. The conjugation of such variants leads to AOCs having a DAR of 4, in which both heavy chains are specifically functionalized at positions Q295 and Q297.

In embodiments, the chemical modification strategy utilized to create ADCs described herein is lysine conjugation. Lysine residues in proteins, e.g., antibodies, possess a primary amine group (—NH₂) in their side chains, making them suitable targets for chemical modification. This primary amine group can react with various chemical reagents, including small molecules or polymers (e.g., cleavable and non-cleavable linkers).

In embodiments, lysine conjugation can be either site-specific or random. In site-specific conjugation, specific lysine residues within a protein, e.g., antibody, can be targeted ensuring precise control over the modification. In contrast, random conjugation involves modifying lysine residues without selectivity.

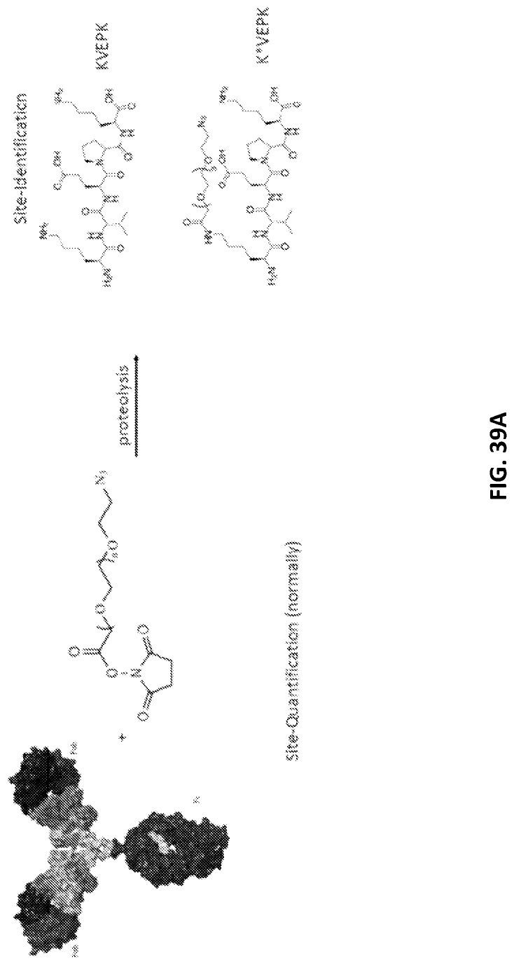

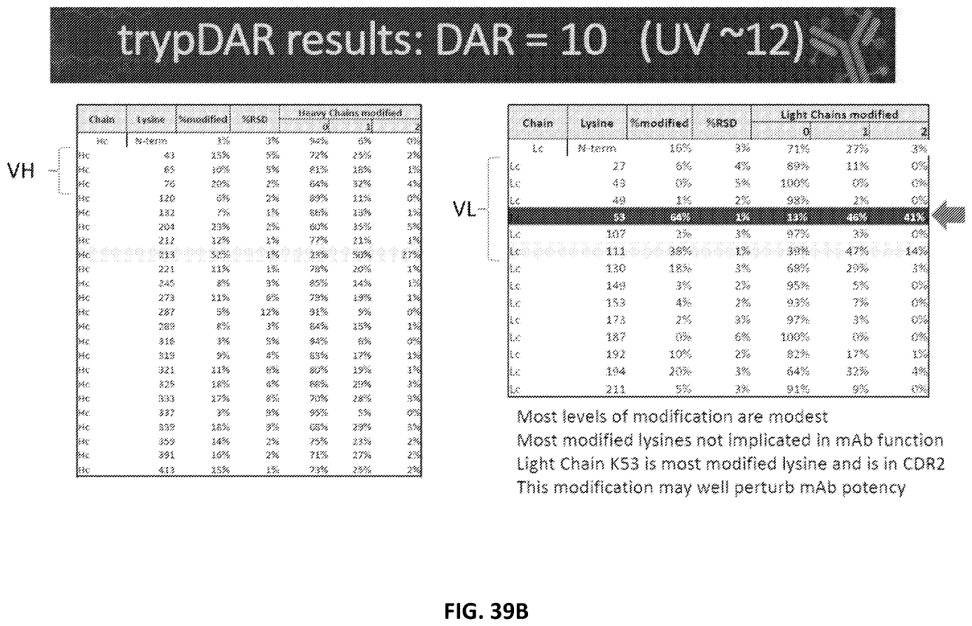

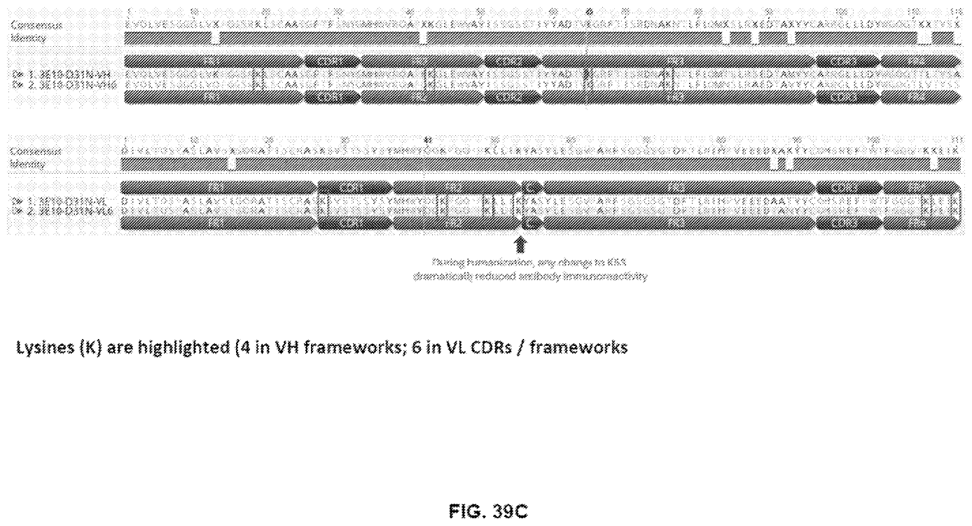

However, as described in Example 26 and FIGS. 39A-39C, tryptic peptide mapping of 3E10 conjugated at lysine residues showed that V region lysines in both VL and VH were observed to be modified and, in fact, VL K53 is the most modified lysine, occurred in nearly⅔ of instances. Furthermore, the humanization trials described in WO 2023/168352, demonstrated that any modification to VL K53 negatively impacted the immunoreactivity to nucleic acids of the humanized 3E10-D31N monoclonal antibody (V66). This is further elucidated in Examples 27, which show greater uptake of 3E10-D31N monoclonal antibody (V66) oligonucleotide conjugates utilizing transglutaminase-mediated enzymatic conjugation in A427 cells, and in Example 28, which utilize hindered disulfide linker (SPDMV) conjugate (transglutaminase-SPDMV), and imparted the highest exon-skipping (~15%), representing a greater than 10× improvement over lysine-PEG8 AOC.

In embodiments, the conjugation strategy is selected from one of the following:

| ID No. | Conjugation strategy |
|---|---|
| CS1 | Lysine conjugation (Lys) |
| CS1A | Random lysine conjugation |
| CS1B | Site-selective lysine conjugation (Lys-azide) |
| CS2 | Transglutaminase-mediated enzymatic conjugation (Tg) |
| CS3 | Amine conjugation |
| CS4 | Thiol conjugation |
| CS5 | Ester linkage |

a. Lysine Attachment

In some embodiments of the ADCs described herein, the linker moiety is conjugated to the antibody or antigen binding fragment therein through an amine linkage at one or more surface-exposed lysine residues on the antibody or antigen binding fragment thereof. Generally, lysine conjugation is a random process, with respect to which lysines are conjugated to the liker-payload. However, some preference for conjugation at certain lysines can occur due to the context of the primary, secondary, ternary, and/or quaternary structure surrounding a particular lysine residue. Many different chemistries are known in the art for attaching payloads to proteins at lysine groups. For example, activated esters on the drug-linker complexes, often O-succinimide reagents such as N-hydroxysuccinimidyl (NHS) or sulfo-NHS esters, can react with the antibody lysine residues and achieve conjugation via amide bonds, or stable amidine bonds can be generated on an antibody by the reaction of imido ester compounds, such as Traut's reagent, with antibody lysine residues. Further description of lysine conjugation techniques are described, for example, in Walker, J. M., et al., “Antibody-drug conjugates,” Humana Press (2013); Bhat, A. S., et al., “The next step in homogenous bioconjugate development: optimizing payload placement and conjugate composition,” BioProcess International (2014); and Jain, N., et al., “Current ADC linker chemistry,” Pharm Res., 32:3526-40 (2015), the disclosure of which are incorporated herein by reference, in their entireties, for all purposes.

Accordingly, in some embodiments, the present disclosure provides pharmaceutical compositions comprising an antibody-drug conjugate (ADC) having the formula A-(L-Pᵣ)_{q}, where: A is a 3E10 antibody or antigen-binding fragment thereof as described herein, L is a linker as described herein, P is a payload moiety as described herein, r is an integer from 1 to 4, and q is an integer from 1 to 16, in which L is conjugated to A through lysine moieties.

Advantageously, because the structural regions of antibodies contain many lysine residues, lysine conjugation can be used to generate ADC molecules with a high drug to antibody ratio (DAR). Accordingly, in some embodiments, a composition comprising an ADC where lysine attachment is used to conjugate the drug to the antibody or antigen binding fragment thereof as described herein will have an average DAR of at least 4 (an average of at least four drug moieties attached to each antibody). In some embodiments, such a composition will have an average DAR of at least 6. In some embodiments, such a composition will have an average DAR of at least 8. In some embodiments, such a composition will have an average DAR of at least 10. In some embodiments, such a composition will have an average DAR of at least 2, at least 3, at least 4, at least 5, at least 6, at least 7, at least 8, at least 9, at least 10, at least 11, at least 12, or more.

Accordingly, in some embodiments, a composition comprising an ADC where lysine attachment is used to conjugate the drug to the antibody or antigen binding fragment thereof as described herein, r is 1 and q is at least 4. In some embodiments, r is 1 and q is at least 6. In some embodiments, r is 1 and q is at least 8. In some embodiments, r is 1 and q is at least 10. In some embodiments, r is 1 and q is at least 2, at least 3, at least 4, at least 5, at least 6, at least 7, at least 8, at least 9, at least 10, at least 11, at least 12, or more. In some embodiments, a composition comprising an ADC where lysine attachment is used to conjugate the drug to the antibody or antigen binding fragment thereof as described herein, r is 2 (e.g., the linker is a branched linker) and q is at least 2. In some embodiments, r is 2 and q is at least 3. In some embodiments, r is 2 and q is at least 4. In some embodiments, r is 2 and q is at least 5. In some embodiments, r is 2 and q is at least 2, at least 3, at least 4, at least 5, at least 6, at least 7, at least 8, at least 9, at least 10, at least 11, at least 12, or more. In yet other embodiments, a composition comprising an ADC where lysine attachment is used to conjugate the drug to the antibody or antigen binding fragment thereof as described herein, the linker is highly branched, e.g., r is at least 3. In some embodiments, r is at least 4. In some embodiments, r is at least 5, 6, 7, 8, or more.

b. Cysteine Attachment

In some embodiments of the ADCs described herein, the linker moiety is conjugated to the antibody or antigen binding fragment therein through a sulfide linkage at one or more surface-exposed cysteine residues on the antibody or antigen binding fragment thereof. In some embodiments of the ADCs described herein, the linker moiety is conjugated to the antibody or antigen binding fragment therein through a thiol side chain of one or more cysteine residues on the antibody or antigen binding fragment thereof. In general, antibodies do not possess free thiols, and all cysteine residues form disulfide bonds. In human IgG1, which is commonly used in modern ADCs, there are 4 interchain and 12 intrachain disulfide bonds. The 4 interchain disulfides, which are generally not critical for structural stability of IgG1, can be selectively reduced under mild conditions to give 2, 4, 6, or 8 free thiols while keeping the 12 intrachain disulfides intact. Due to the limited number of conjugation sites and the distinct reactivity of the thiol group, cysteine-based conjugation allows for controlled DAR and heterogeneity. Engineered Cys residues can also be used for site specific conjugation without the partial reduction of the endogenous disulfide bonds using, e.g., EnCys-mAb technology. Many different chemistries are known in the art for attaching payloads to proteins at cystine groups. For example, 8 nucleophilic cysteine residues can first be liberated from the reduced inter-chain disulfide bonds via reducing agents and later conjugated with drug-linker complexes. This approach generates ADCs with heterogeneous conjugation sites and a different number of drugs attached, resulting in a drug to antibody ratio (DAR) ranging from 0-8. Alternatively, partial reduction with either dithiothreitol (DTT) or tris(2-carboxyethyl) phosphine (TCEP) can be used to result in the disruption of the heavy-light inter-chain disulfides to release free Cys for drug conjugation, while treatment using 5,5′-dithiobis (2-nitrobenzoic acid) (DTNB) yields drug conjugates connect to the Cys residues usually involved in heavy-heavy inter-chain disulfides. Further description of cysteine conjugation techniques are described, for example, in

Behrens, C. R.; et al., “Methods for site-specific drug conjugation to antibodies,” mAbs. 2014, 6 (1): 46-53; Dennler, P.; et al., “Antibody conjugates: from heterogeneous populations to defined reagents,” Antibodies. 2015, 4: 197-224; and Agarwal, P.; et al., “Site-specific antibody-drug conjugates: the nexus of bioorthogonal chemistry, protein engineering, and drug development,” Bioconjugate Chem. 2015, 26: 176-192, the disclosure of which are incorporated herein by reference, in their entireties, for all purposes.

Accordingly, in some embodiments, the present disclosure provides pharmaceutical compositions comprising an antibody-drug conjugate (ADC) having the formula A-(L-Pᵣ)_{q}, where: A is a 3E10 antibody or antigen-binding fragment thereof as described herein, L is a linker as described herein, P is a payload moiety as described herein, r is an integer from 1 to 4, and q is an integer from 1 to 16, in which L is conjugated to A through cysteine moieties.

Advantageously, because of the limited number of conjugation sites and the distinct reactivity of the thiol group, cysteine-based conjugation can be used to generate ADC molecules with a controlled DAR and heterogeneity.

Accordingly, in some embodiments, a composition comprising an ADC where cysteine attachment is used to conjugate the drug to the antibody or antigen binding fragment thereof as described herein will have an average DAR of at least 2. In some embodiments, such a composition will have an average DAR of at least 3. In some embodiments, such a composition will have an average DAR of at least 4. In some embodiments, such a composition will have an average DAR of at least 5. In some embodiments, such a composition will have an average DAR of at least 6. In some embodiments, such a composition will have an average DAR of at least 7. In some embodiments, such a composition will have an average DAR of about 8. In some embodiments, such a composition will have an average DAR of at least 2, at least 3, at least 4, at least 5, at least 6, at least 7, or about 8.

Accordingly, in some embodiments, a composition comprising an ADC where cysteine attachment is used to conjugate the drug to the antibody or antigen binding fragment thereof as described herein, r is 1 and q is at least 2. In some embodiments, r is 1 and q is at least 3. In some embodiments, r is 1 and q is at least 4. In some embodiments, r is 1 and q is at least 5. In some embodiments, r is 1 and q is at least 6. In some embodiments, r is 1 and q is at least 7. In some embodiments, r is 1 and q is about 8. In some embodiments, r is 1 and q is at least 2, at least 3, at least 4, at least 5, at least 6, at least 7, or 8. In some embodiments, a composition comprising an ADC where cysteine attachment is used to conjugate the drug to the antibody or antigen binding fragment thereof as described herein, r is 2 (e.g., the linker is a branched linker) and q is at least 2. In some embodiments, r is 2 and q is at least 3. In some embodiments, r is 2 and q is at least 4. In some embodiments, r is 2 and q is at least 5. In some embodiments, r is 2 and q is at least 6. In some embodiments, r is 2 and q is at least 7. In some embodiments, r is 2 and q is about 8. In some embodiments, r is 2 and q is at least 2, at least 3, at least 4, at least 5, at least 6, at least 7, or about 8. In yet other embodiments, a composition comprising an ADC where cysteine attachment is used to conjugate the drug to the antibody or antigen binding fragment thereof as described herein, the linker is highly branched, e.g., r is at least 3. In some embodiments, r is at least 4. In some embodiments, r is at least 5, 6, 7, 8, or more.

c. Transglutaminase-Based Attachment

In some embodiments of the ADCs described herein, the linker moiety is conjugated to the antibody or antigen binding fragment therein through the primary amide side chain of one or more glutamine residues on the antibody or antigen binding fragment thereof. Generally, transglutaminase derived from Streptomyces mobaraensis catalyzes transpeptidation where a primary amine-containing linker is covalently attached to the primary amide side chain of a specific glutamine (Q295) within deglycosylated antibodies, resulting in ADCs with a defined DAR of 2 (one conjugation site per heavy chain) (Jeger et al., “Site-specific and stoichiometric modification of antibodies by bacterial transglutaminase,” Angew Chem Int Ed Engl 2010 49:9995-9997; and Dennler et al., “Transglutaminase-based chemo-enzymatic conjugation approach yields homogeneous antibody-drug conjugates,” Bioconjugate Chem 2014 25:569-578 the disclosure of which are incorporated herein by reference, in their entireties, for all purposes). An N297Q mutation prior to this conjugation provides two more reaction sites (DAR=4). This method is quite advantageous in terms of practical ADC production as the glycosidase and transglutaminase directly modify and conjugate native mAbs with the payload, without the need for genetic engineering. An alternative version using a peptide sequence-specific transglutaminase can also be used (Strop et al., “Location matters: site of conjugation modulates stability and pharmacokinetics of antibody drug conjugates,” Chem Biol 2013 20:161-167). This enzyme recognizes and utilizes LLQG motif that is genetically incorporated, resulting in site-specific antibody-drug conjugation.

Accordingly, in some embodiments, the present disclosure provides pharmaceutical compositions comprising an antibody-drug conjugate (ADC) having the formula A-(L-Pᵣ)_{q}, where: A is a 3E10 antibody or antigen-binding fragment thereof as described herein, L is a linker as described herein, P is a payload moiety as described herein, r is an integer from 1 to 4, and q is an integer from 1 to 16, in which L is conjugated to A through glutamine moieties.

Accordingly, in some embodiments, a composition comprising an ADC where glutamine attachment is used to conjugate the drug to the antibody or antigen binding fragment thereof as described herein will have an average Drug Antibody Ratio (DAR) of about 2. In some embodiments, such a composition will have an average DAR of at least 2. In some embodiments, such a composition will have an average DAR of at least 3. In some embodiments, such a composition will have an average DAR of about 4. In some embodiments, such a composition will have an average DAR of about 2, at least 2, at least 3, or about 4.

Accordingly, in some embodiments, a composition comprising an ADC where glutamine attachment is used to conjugate the drug to the antibody or antigen binding fragment thereof as described herein, r is 1 and q is about 2. In some embodiments, r is 1 and q is at least 2. In some embodiments, r is 1 and q is at least 3. In some embodiments, r is 1 and q is about 4. In some embodiments, r is 1 and q is about 2, at least 2, at least 3, or about 4. In some embodiments, a composition comprising an ADC where glutamine attachment is used to conjugate the drug to the antibody or antigen binding fragment thereof as described herein, r is 2 (e.g., the linker is a branched linker) and q is about 2. In some embodiments, r is 2 and q is at least 2. In some embodiments, r is 2 and q is at least 3. In some embodiments, r is 2 and q is about 4. In some embodiments, r is 2 and q is about 2, at least 2, at least 3, or about 4. In yet other embodiments, a composition comprising an ADC where cysteine attachment is used to conjugate the drug to the antibody or antigen binding fragment thereof as described herein, the linker is highly branched, e.g., r is at least 3. In some embodiments, r is at least 4. In some embodiments, r is at least 5, 6, 7, 8, or more. 2. Cleavable Linkers

In embodiments, the linker is a cleavable linker. As used herein, “cleavable linker” refers to a linker which can connect two or more molecules and then be cleaved once exposed to an agent. Cleavable linkers can include chemically or enzymatically unstable or degradable linkages. Cleavable linkers generally rely on processes inside the cell to liberate the drug, such as reduction in the cytoplasm, exposure to acidic conditions in the lysosome, or cleavage by specific proteases or other enzymes within the cell. Cleavable linkers generally incorporate one or more chemical bonds that are either chemically or enzymatically cleavable while the remainder of the linker is non-cleavable. In embodiments, the cleavable linker is an acid-labile linker (Linker ID NO. 215), a phosphatase linker (Linker ID NO. 216), glucuronidase linker (Linker ID NO. 217), a cathepsin-B cleavable linker (Linker ID NO. 218), a cathepsin-L cleavable linker (Linker ID NO. 339), a protease-sensitive linker (Linker ID NO. 219), a photo-labile linker (Linker ID NO. 220), or a disulfide (SPDMV) transglutaminase-containing linker (Linker ID NO. 221). The cleavable linker can be selected from the group consisting of: succinyl (Linker ID NO. 222), 0-succinyl (Linker ID NO. 223), 4-succinimidyl-oxycarbonyl-α-(2-pyridyldithio)toluene (Linker ID NO. 224), sulfosuccinimidyl 6-(3′-(2-pyridyldithio)propionamido)hexanoate (Linker ID NO. 225), N-succinimidyl-3-(-2-pyridyldithio)-proprionate (Linker ID NO. 226), succinimidyl 6-(3(2-pyridyldithio)propionamido)hexanoate (Linker ID NO. 227), 3-(2-pyridyldithio)-propionyl hydrazide, S-(2-thiopyridyl)-L-cysteine (Linker ID NO. 228), N-succinimidyl 4-(2-pyridyldithio)butanoate (SPDB) (Linker ID NO. 229), N-succinimidyl 4-(2-pyridyldithio)-2-sulfobutanoate (sulfo-SPDB) (Linker ID NO. 230), N-succinimidyl 4-(2-pyridyldithio)pentanoate (SPP) (Linker ID NO. 231), CL2A (Linker ID NO. 232), maleimidocaproyl-valine-citrulline-p-aminobenzoyloxycarbonyl (MC-vc-PAB) (Linker ID NO. 233), and N-succinimidyl 4-(2-pyridyldithio)-2-sulfopentanoate (sulfo-SPP) (Linker ID NO. 234).

Additional examples of linkers which contain cleavable disulfide bonds include, but are not limited to “DPDPB”, 1,4-di-[3′-(2′-pyridyldithio) propionamido]butane (Linker ID NO. 236); “SADP”, (N-succinimidyl (4-azidophenyl) 1,3′-dithio- propionate) (Linker ID NO. 237); “Sulfo-SADP” (Sulfosuccinimidyl (4-azi- dophenyldithio) propionate (Linker ID NO. 238); “DSP”-Dithio bis (succinimidylproprionate) (Linker ID NO. 239); “DTSSP”-3,3′-Dithio bis (sulfosuccinimidylpropionate) (Linker ID NO. 240); “DTBP”-dimethyl 3,3dithiobispropionimidate-2HCI (Linker ID NO. 241). Examples of linkers cleavable by oxidation include “DST”-disuccinimidyl tartarate (Linker ID NO. 242); and “Sulfa-DST”-disuccin- imidyl tartarate (Linker ID NO. 261).

In some embodiments, the linker L is selected from:

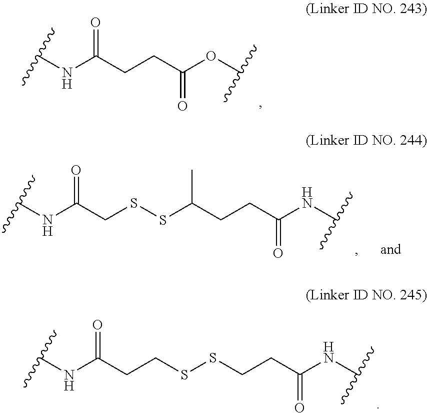 [SMILES: *NC(=O)CCC(=O)O* ; *NC(=O)CCC(C)SSCC(=O)N* ; *NC(=O)CCSSCCC(=O)N*]

In some embodiments, the linker L is selected from:

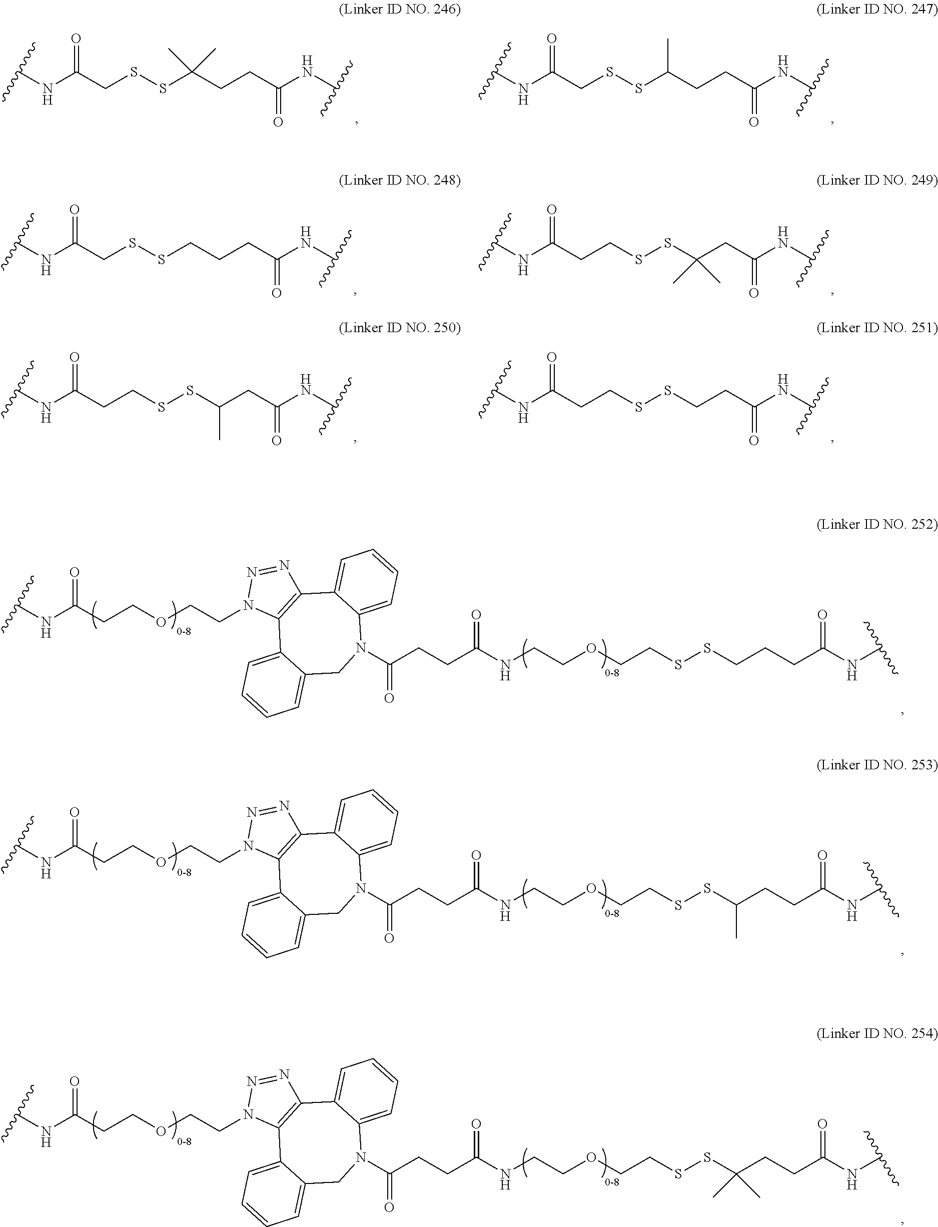

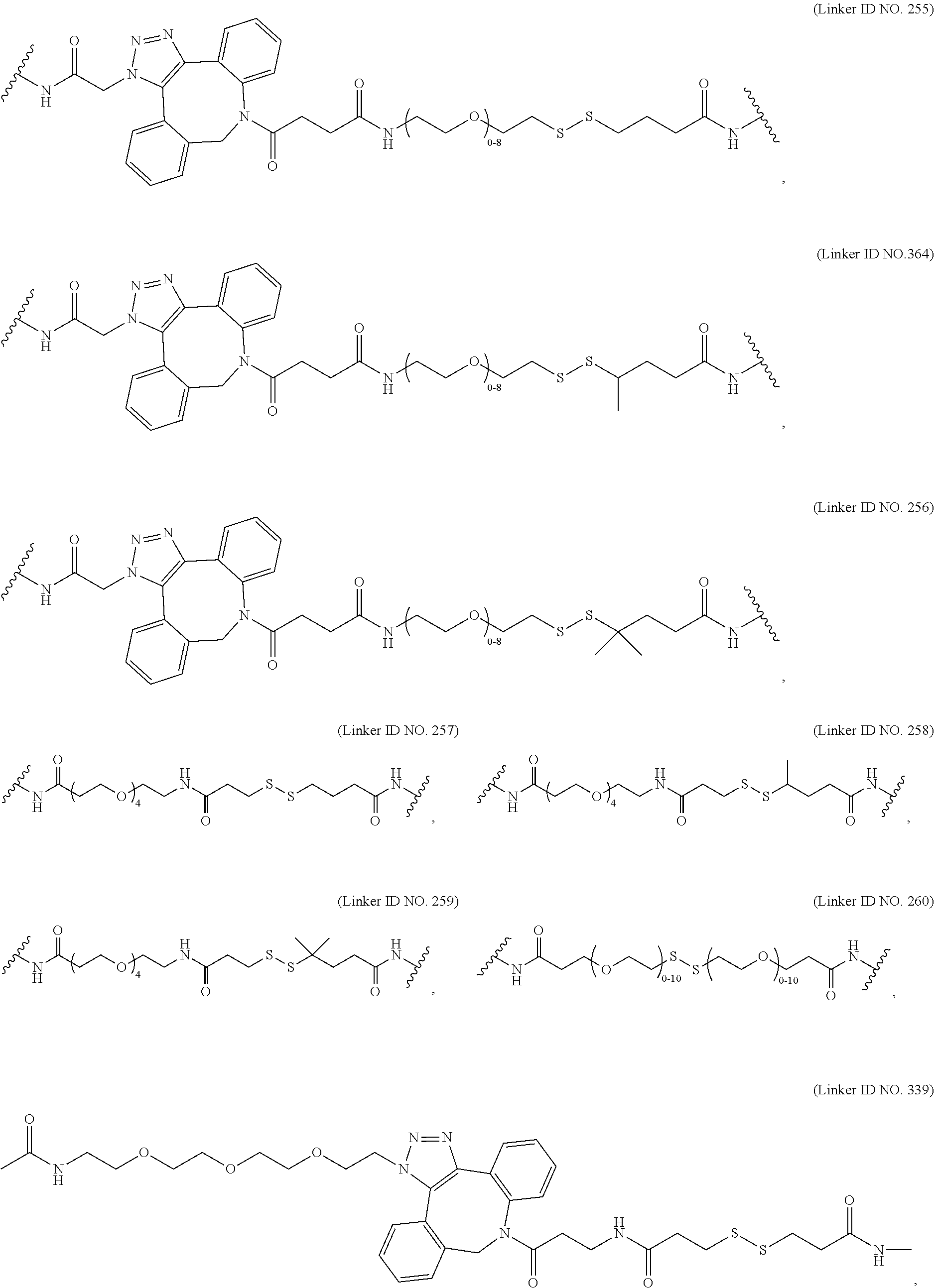

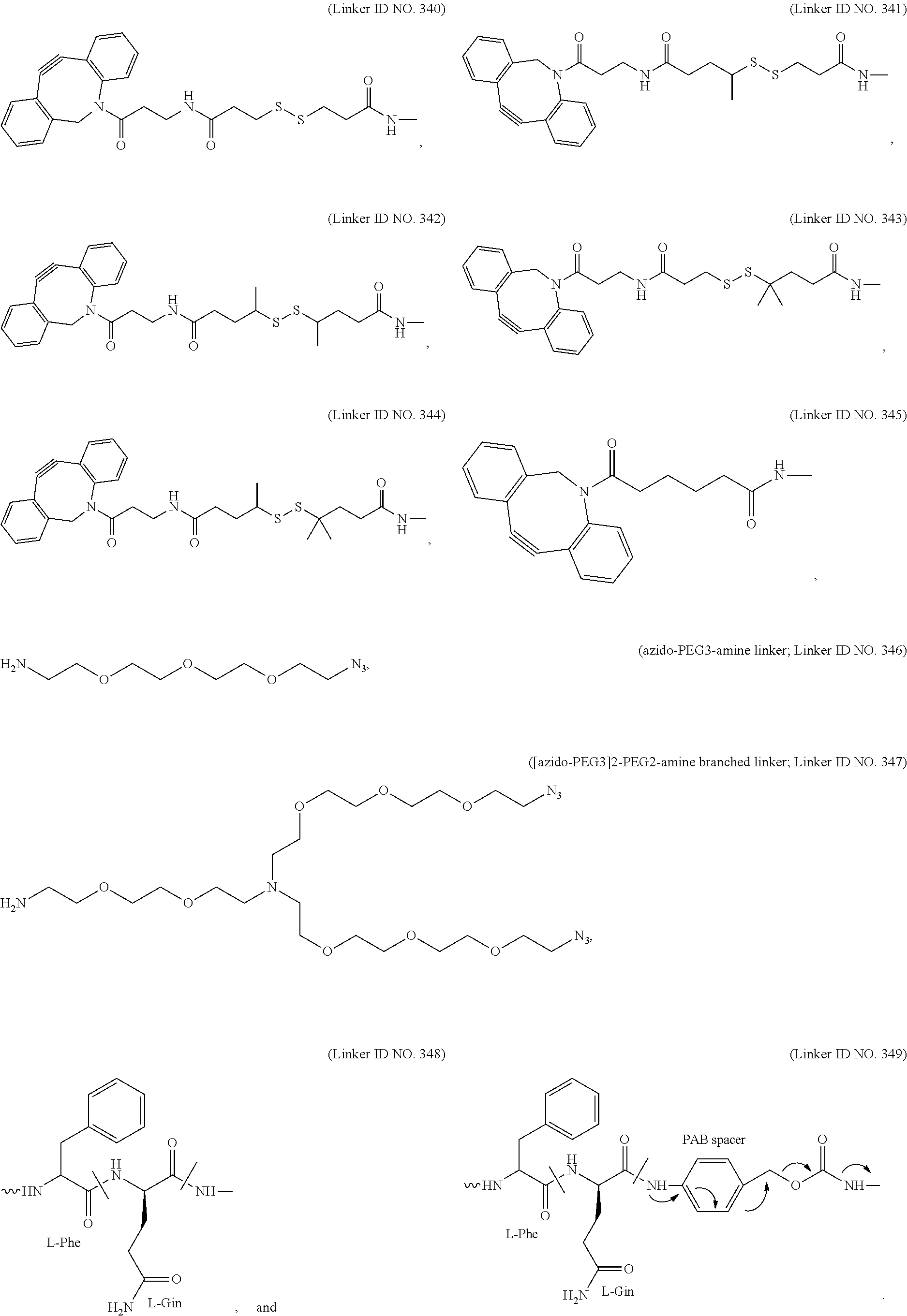

a. Cathepsin-Cleavable Linkers

In some embodiments of the ADCs described herein, the linker moiety is conjugated to the antibody or antigen binding fragment therein through a cathepsin-cleavable linker.

ADCs can enter cells via receptor-mediated endocytosis, during intracellular transit or trafficking, and thus they may ultimately encounter the acidic degradative environment of the lysosome which contains multiple proteases and other catalytic enzymes for breakdown of internalized biologic substances. Most often, cleavable linkers therefore leverage an attribute of the lysosome for payload release such as lability to low pH environment, disulfide reducing environment, or cleavage by a lysosomal protease such as cathepsin B. Protease cleavable linkers or (peptide linkers) typically contain a dipeptide sequence based on deduced canonical cleavage specificity for a given protease. Common dipeptide sequences are Valine-Citrulline or Valine-Alanine. These linkers have been used frequently for conjugation of both ADCs and AOCs.

Upon release via linker cleavage, the payloads then require escape from the endosome to traffic to the specific subcellular region in order to mediate an effect: ADCs: e.g. cell killing agents (cytosol-tubulin inhibitors such as auristatin or maytansine; or nuclear—direct or indirect DNA damaging agents such as duocarmycin or topoisomerase inhibitors).

3E10 and its derivatives, including the humanized V66 IgG1κ, are anti-DNA antibodies with the unique property of direct cell entry and subsequent trafficking to the nucleus of cells. This occurs via interaction of antibody:DNA complexes with the cell surface transporter ENT2 (equilibrative nucleoside transporter-2) and so does require the classical endocytic mechanism employing clathrin or dynamin mediated internalization. Further, upon internalization, the antibody does not encounter early or late endosomes and, importantly, does not encounter the harsh environment of the lysosome. As a result, trafficking occurs directly to the nucleus. As such, 3E10 and its derivatives can serve as delivery vehicles for therapeutic payloads that are mechanistically active within the nucleus of target cells.

A challenge for generating an effective ADC with 3E10 or a derivative (e.g. V66) is that without encountering the lysosome and its acidic environment and battery of degradative enzymes, it is not clear what enzymes the conjugate may encounter en route or within the nucleus to deliver its cargo. In embodiments, an ADC described herein comprises a cleavable linker for which the catalytic agent is present within the within the nucleus for selective release within that organelle.

It has been found that the cysteine protease Cathepsin L can also be present within the nucleus of cells. See, e.g., Goulet et al., “Increased expression and activity of nuclear cathepsin L in cancer cells suggests a novel mechanism of cell transformation,” Mol Cancer Res. 2007 September; 5(9):899-907, incorporated herein by reference in its entirety. This ubiquitous protease has previously been shown to be present primarily in lysosomes as well as in secreted form. Translation initiation within the cathepsin L mRNA for both mouse and human cells has been shown to take place at alternative internal start sites resulting in a polypeptide lacking an NH₂-terminal signal peptide. Other cathepsins typically identified in lysosomes have also been identified in the nucleus, including cathepsin D, cathepsin V, and cathepsin B.

A literature review identified the existence of a nuclear form of the cysteine protease cathepsin-L in mouse tumor cells (and also in human tumor cells) (Goulet et al., “Increased expression and activity of nuclear cathepsin L in cancer cells suggests a novel mechanism of cell transformation,” Mol Cancer Res. 2007 September; 5(9):899-907; and Soond et al., “Lost or Forgotten: The nuclear cathepsin protein isoforms in cancer,” Cancer Lett. 2019 Oct. 10; 462:43-50, each incorporated herein by reference in their entireties. Indeed there have been identified several proteases identified that localize to the nucleus but cathepsin-L is most prominent. Further, in cancer cells cathepsin-L appears to play a significant role in cell cycle progression and importantly tumor progression and metastasis.

In some embodiments, ADCs disclosed herein comprise dipeptides that are substrates for proteolytic cleavage by nuclear localized cathepsins, optionally cathepsin L, cathepsin S, cathepsin D, cathepsin V, and/or cathepsin B.

In some embodiments, the present disclosure provides an antibody-drug conjugate (ADC) having the formula A-(L-Pᵣ)_{q}, where: A is a 3E10 antibody or antigen-binding fragment thereof as described herein (e.g., as described in the section titled 3E10 Antibodies and Antigen-Binding Fragments), L is a cathepsin-cleavable linker, P is a payload moiety, r is an integer from 1 to 4 and q is an integer from 1 to 16. In some embodiments, L is a cathepsin L-cleavable linker. In some embodiments, L is a cathepsin S-cleavable linker. In some embodiments, L is a cathepsin D-cleavable linker. In some embodiments, L is a cathepsin B-cleavable linker. In some embodiments, L is a cathepsin V-cleavable linker.

In embodiments, L comprises a dipeptide selected from Phe-Gin (Linker ID NO. 350), Val-Gin (Linker ID NO. 351), Leu-Gln (Linker ID NO. 352), Tyr-Met (Linker ID NO. 353), Phe-Arg (Linker ID NO. 354), Phe-Gly (Linker ID NO. 355), Trp-Thr (Linker ID NO. 356), Tyr-Gly (Linker ID NO. 357), Phe-Thr (Linker ID NO. 358), Val-Gly (Linker ID NO. 359), Val-Cit (Linker ID NO. 360), Val-Arg (Linker ID NO. 361), Thr-Thr (Linker ID NO. 362), and Val-Thr (Linker ID NO. 363). In embodiments, L further comprises one or more PEG molecules.

### 3. Non-Cleavable Linkers

In embodiments, the linker is a non-cleavable linker. In embodiments, L is a non-cleavable linker, and eventual breakdown of the antibody releases the payload from the ADC. As used herein, “non-cleavable linker” refers to linkers where the release of the biologically active molecule does not depend on, for example, the differential properties between the plasma and some cytoplasmic compartments, or whether the linker has a physical property that permits enzymatic cleavage or chemical cleavage. Non-cleavable linkers can be alkylene chains, or can be polymeric in nature, such as, for example, those based upon polyalkylene glycol polymers, amide polymers, or can include segments of alkylene chains, polyalkylene glycols and/or amide polymers. Suitable non-cleavable linkers can include N-succinimidyl (4-iodoacetyl)-aminobenzoate (Linker ID NO. 262), sulfosuccinimidyl(4-iodoacetyl)-aminobenzoate (Linker ID NO. 263), dichlorotriazinic acid (Linker ID NO. 264), N-succinimidyl-[(N-maleimidopropionamido)-tetraethyleneglycol]ester (NHS-PEG4-maleimide) (Linker ID NO. 265), N-succinimidyl 4-(maleimidomethyl) cyclohexanecarboxylate (SMCC) (Linker ID NO. 266), or N-sulfosuccinimidyl 4-(maleimidomethyl) cyclohexanecarboxylate (sulfoSMCC) (Linker ID NO. 267).

Additional examples of non-cleavable linkers are “Sulfo-LC-SMPT”-(sulfosuccinimidyl 6-[alphamethyl-alpha-(2-pyridylthio) toluamido}hexanoate (Linker ID NO. 268); “SMPT”- 4-succinimidyloxycarbonyl-alpha-methyl-α(2-pyridyldithio)toluene (Linker ID NO. 269); “ABH”-Azidobenzoyl hydrazide (Linker ID NO. 270); “NHS-ASA”-N-Hydroxysuccinimidyl-4-azi- dosalicyclic acid (Linker ID NO. 271); “SASD”-Sulfosuccinimidyl 2-(pazidosali- cylamido)ethyl-1,3-dithiopropionate (Linker ID NO. 272); “APDP”-N-{4-(p-azi-dosalicylamido) buthy}-3′ (2′-pyidyldithio)propionamide (Linker ID NO. 273); “BASED”-Bis-[-(4-azidosalicylamido)ethyl]disulfide (Linker ID NO. 274); “HSAB”-N-hydroxysuccinimidyl-4 azidobenzoate (Linker ID NO. 275); “APG”- p-Azidophenyl glyoxal monohydrate (Linker ID NO. 276); “SANPAH”-N-Suc- ciminidyl-6 (4′-azido-2′-mitrophenyl-amimo) hexanoate (Linker ID NO. 277); “Sulfo-SANPAH”-Sulfosuccinimidy16-(4′-azido-2′-nitro-phenylamino) hexanoate (Linker ID NO. 278); “ANB-NOS” N-5-Azido-2-ni- trobenzyoyloxysuccinimide (Linker ID NO. 279); “SAND”-Sulfosuccinimidyl-2- (m-azido-o-mitrobenzamido)-ethyl-1,3′-dithiopr-opionate (Linker ID NO. 280); “PNP-DTP”-p-nitrophenyl-2-diazo-3,3,3-trifluoropropi- onate (Linker ID NO. 281); “SMCC”-Succinimidyl-4-(N-maleimidomethyl)cy- clohexane-1-carboxylate (Linker ID NO. 282); “Sulfo-SMCC”-Sulfosuccinim- idyl-4-(N-maleimidomethyl)cyclohexane-1-carboxy-late (Linker ID NO. 283); “MBS” m-Maleimidobenzoyl-N-hydroxysuccinimide ester (Linker ID NO. 284); “sulfo-MBS”-m-Maleimidobenzoyl-N-hydroxysulfosuccin- imide ester (Linker ID NO. 285); “SIAB”-N-Succinimidyl (4-iodoacetyl)ami- nobenzoate (Linker ID NO. 286); “SulfSIAB”-N-Sulfosuccinimidyl (4-io- doacetyl)aminobenzoate (Linker ID NO. 287); “SMPB”-Succinimidyl 4-(pmalenimidophenyl) butyrate (Linker ID NO. 288); “Sulfo-SMPB”-Sulfosuc- cinimidyl 4-(p-malenimidophenyl) butyrate (Linker ID NO. 289); “DSS”-Disuc-cinimidyl suberate (Linker ID NO. 290); “BMH”-Bis maleimidohexane (Linker ID NO. 291); “BSSS”-bis(sulfosuccinimidyl) suber- ate (Linker ID NO. 292); “DFDNB”-1,5-difluoro- 2,4-dinitrobenzene (Linker ID NO. 293); “DMA”-dimethyl adipimidate 2HCI (Linker ID NO. 294); “DMP”-Dimethyl pimelimidate-2HCI (Linker ID NO. 295); “DMS”-dimethyl suberimidate-2-HCl (Linker ID NO. 296); “SPDPN-succinimidyl-3-(2-py-ridylthio) propionate (Linker ID NO. 297); “Sulfo-HSAB”-Sulfosuccinimidyl 4-(pazidophenyl) butyrate (Linker ID NO. 298); “Sulfo-SAPB”-Sulfosuccinim- idyl 4-(p-azidophenylbutyrate) (Linker ID NO. 299); “ASIB”-1-9p-azidosalicyla-mido)-4-(iodoacetamido) butane (Linker ID NO. 300); “ASBA”-4-(p-Azidosali-cylamido) butylamine (Linker ID NO. 301).

Bifunctional chemical linkers include 4-succinimidyl-oxycarbonyl-C-(2-pyridyldithio) toluene (Linker ID NO. 302); sulfosuccinimidyl-6-O-methyl-O-(pyridyldithiol)-toluamidohe-xanoate (Linker ID NO. 303); N-succinimidyl-3(2-pyridyldithio)-proprionate (Linker ID NO. 304); succinimidyl-6-3(-(-2-pyridyldithio)-proprionamidohexanoate (Linker ID NO. 305); sulfosuccinimidyl-6-3 (-(-2-pyridyldithio)-propionamido hexanoate (Linker ID NO. 306); 3-(2-pyridyldithio)-propionyl hydrazide (Linker ID NO. 307), Ellman's reagent (Linker ID NO. 308), dichlorotriazinic acid (Linker ID NO. 309), S-(2-thiopyridyl)-Lcysteine (Linker ID NO. 310), and the like. Further bifunctional linking molecules are disclosed in U.S. Pat. Nos. 5,349,066; 5,618,528; 4,569,789; 4,952,394; and 5,137,877, each of which is incorporated herein by reference in its entirety.

There are a large number of chemical cross-linking agents that are known to those skilled in the art and useful for cross-linking portions of a conjugate. For example, the cross-linking agents are heterobifunctional cross-linkers, which can be used to link molecules in a stepwise manner. Heterobifunctional cross-linkers provide the ability to design more specific coupling methods for conjugating, thereby reducing the occurrences of unwanted side reactions such as homo-protein polymers.

A wide variety of heterobifunctional cross-linkers are known in the art, including succinimidyl 4-(N-maleimidomethyl) cyclohexane-1-carboxylate (SMCC) (Linker ID NO. 311), m-Maleimidobenzoyl- N-hydroxysuccinimide ester (MBS) (Linker ID NO. 312); N-succinimidyl (4-io-doacetyl) aminobenzoate (SIAB) (Linker ID NO. 313); succinimidyl 4-(p-ma-30 leimidophenyl) butyrate (SMPB) (Linker ID NO. 314); 1-ethyl-3-(3- dimethylaminopropyl) carbodiimide hydrochloride (EDC) (Linker ID NO. 315); 4-succinimidyloxycarbonyl-a-methyl-a-(2-pyridyldithio)- tolune (SMPT) (Linker ID NO. 316); N-succinimidyl 3-(2-pyridyldithio) propi-onate (SPDP) (Linker ID NO. 317); succinimidyl 6-[3-(2-pyridyldithio) propi-onate]hexanoate (LC-SPDP) (Linker ID NO. 318). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exists a number of other cross-linking agents including homobifunctional and photoreactive cross-linkers. Disuccinimidyl subcrate (DSS), bismaleimidohexane (BMH) and dimethylpimelimidate-2 HCl (DMP) are examples of useful homobifunctional cross- linking agents, and bis-[B-(4-azidosalicylamido)ethyl]disul- fide (BASED) and N-succinimidyl-6(4′-azido-2′-nitrophe-nylamino)hexanoate (SANPAH) are examples of useful photoreactive cross-linkers.

One class of heterobifunctional cross-linkers, included above, contain the primary amine reactive group, N-hydroxysuccinimide (NHS), or its water soluble analog N-hydroxysulfosuccinimide (sulfa-NHS). Primary amines (lysine epsilon groups) at alkaline pH's are unprotonated and react by nucleophilic attack on NHS or sulfa-NHS esters. This reaction results in the formation of an amide bond, and release of NHS or sulfa-NHS as a by-product. Another reactive group useful as part of a hetero- bifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodo- acetyl functions) react with —SH groups at physiological pH's. Both of these reactive groups result in the formation of stable thioether bonds.

In embodiments, the universal antibody-drug conjugate (ADC) provided herein comprises a linker attachment schema selected from the group consisting of: conjugation to exposed lysines, cysteine conjugation following modest reduction of 3E10, engineering free cysteines (e.g., at the C-terminus), site-specific transglutaminase linkage, wherein the linkage can further comprise click chemistry, and any combination thereof. In embodiments, an ADC provided herein further comprises a linker attachment schema comprising engineering free cysteines (e.g., at the C-terminus). In embodiments, an ADC provided herein, comprises an antibody that is cysteine-engineered at the site of linker attachment. In embodiments, an ADC provided herein, comprises a cleavable linker comprising a cleavable disulfide bond. In embodiments, an ADC provided herein comprises a linker wherein the linker is a hindered linker, wherein the linker comprises a carbon atom bearing a sulfur capable of forming a disulfide bond, and wherein the carbon atom is substituted with at least one substituent other than H. In embodiments, the substituent comprises a hydrocarbyl or a substituted hydrocarbyl moiety.

In some embodiments, the linker L is selected from:

| | Linker |
|---|---|
| Linker | ID NO. |
| 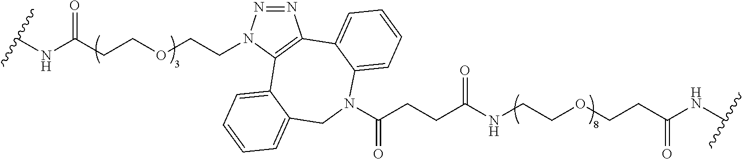 | 101 |
| 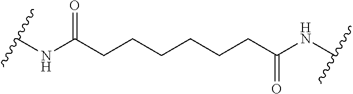 [SMILES: *NC(=O)CCCCCCC(=O)N*] | 102 |
| 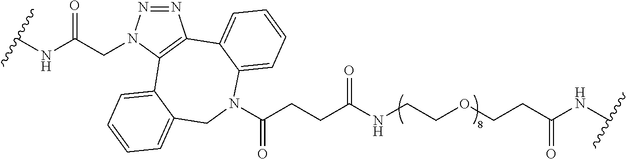 | 103 |
| 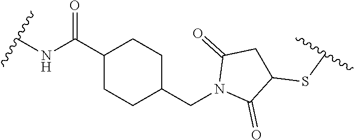 [SMILES: *NC(=O)C1CCC(CN2C(=O)CC(S*)C2=O)CC1] | 104 |
| 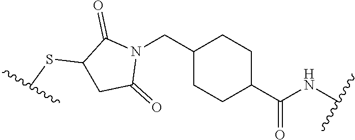 [SMILES: *NC(=O)C1CCC(CN2C(=O)CC(S*)C2=O)CC1] | 105 |
| 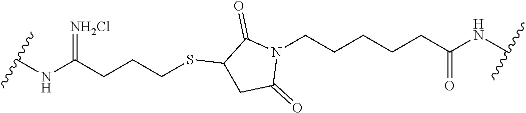 | 106 |
| 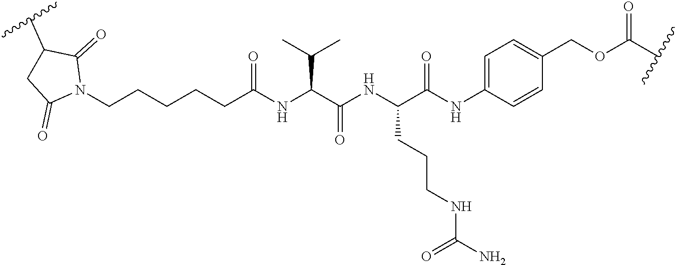 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1] | 107 |
| 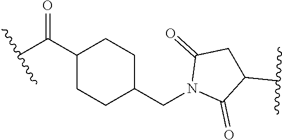 [SMILES: *C(=O)C1CCC(CN2C(=O)CC(*)C2=O)CC1] | 108 |
| 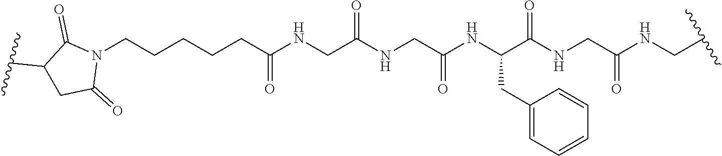 [SMILES: *CNC(=O)CNC(=O)[C@H](Cc1ccccc1)NC(=O)CNC(=O)CNC(=O)CCCCCN1C(=O)CC(*)C1=O] | 109 |
| 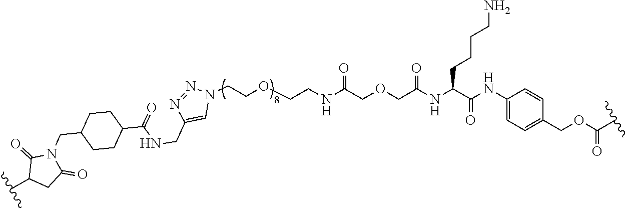 | 110 |
| 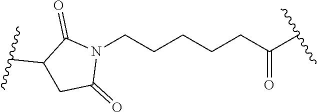 [SMILES: *C(=O)CCCCCN1C(=O)CC(*)C1=O] | 111 |
| 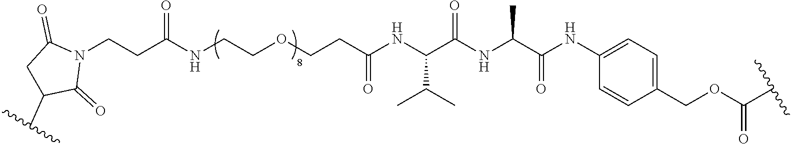 | 112 |
| 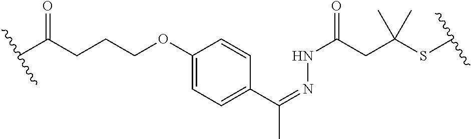 [SMILES: *SC(C)(C)CC(=O)N/N=C(/C)c1ccc(OCCCC(*)=O)cc1] | 113 |
| 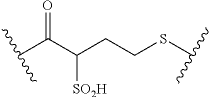 | 114 |
| 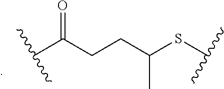 [SMILES: *SC(C)CCC(*)=O] | 115 |
| 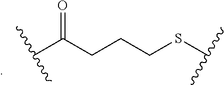 [SMILES: *SCCCC(*)=O] | 116 |
| 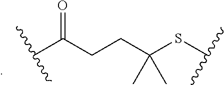 [SMILES: *SC(C)(C)CCC(*)=O] | 117 |
| 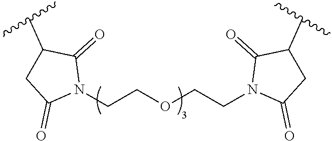 | 118 |
| 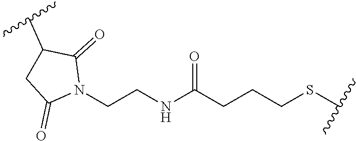 [SMILES: *SCCCC(=O)NCCN1C(=O)CC(*)C1=O] | 119 |
| 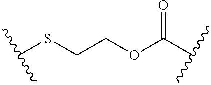 [SMILES: *SCCOC(*)=O] | 120 |
| 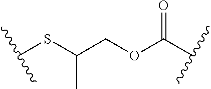 [SMILES: *SC(C)COC(*)=O] | 121 |
| 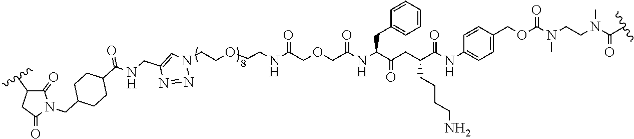 | 122 |
| 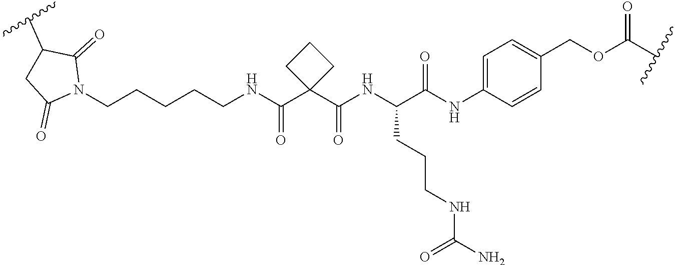 [SMILES: *C(=O)OCc1ccc(NC(=O)[C@H](CCCNC(N)=O)NC(=O)C2(C(=O)NCCCCCN3C(=O)CC(*)C3=O)CCC2)cc1] | 123 |
| 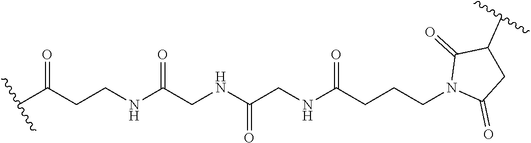 [SMILES: *C(=O)CCNC(=O)CNC(=O)CNC(=O)CCCN1C(=O)CC(*)C1=O] | 124 |
| 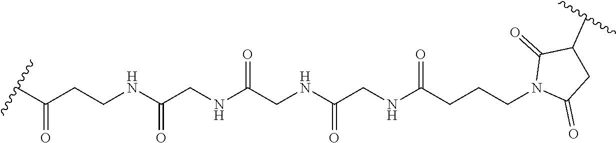 [SMILES: *C(=O)CCNC(=O)CNC(=O)CNC(=O)CNC(=O)CCCN1C(=O)CC(*)C1=O] | 125 |
| 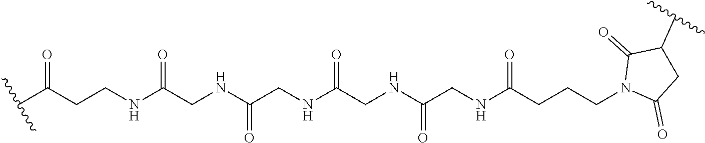 [SMILES: *C(=O)CCNC(=O)CNC(=O)CNC(=O)CNC(=O)CNC(=O)CCCN1C(=O)CC(*)C1=O] | 126 |
| 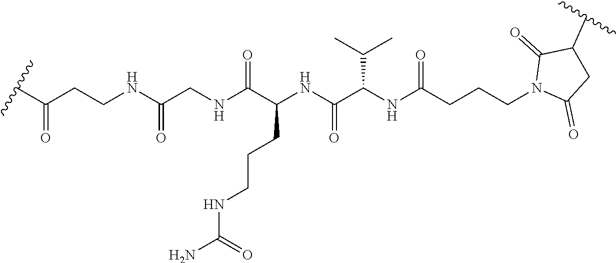 [SMILES: *C(=O)CCNC(=O)CNC(=O)[C@H](CCCNC(N)=O)NC(=O)[C@@H](NC(=O)CCCN1C(=O)CC(*)C1=O)C(C)C] | 127 |
| 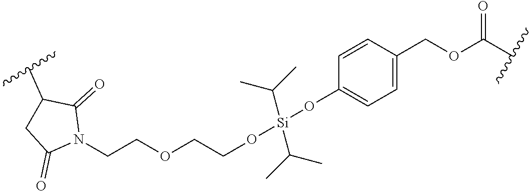 [SMILES: *C(=O)OCc1ccc(O[Si](OCCOCCN2C(=O)CC(*)C2=O)(C(C)C)C(C)C)cc1] | 128 |
| 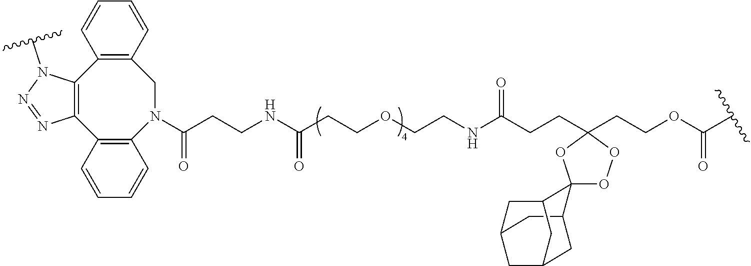 | 129 |
| 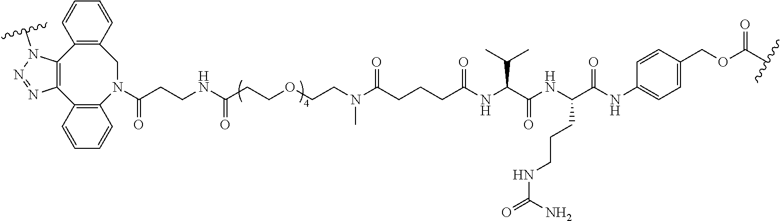 | 130 |
| 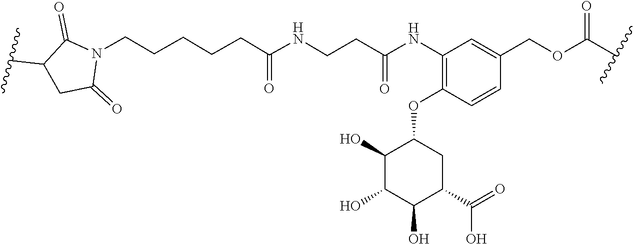 [SMILES: *C(=O)OCc1ccc(O[C@@H]2C[C@H](C(=O)O)[C@@H](O)[C@H](O)[C@H]2O)c(NC(=O)CCNC(=O)CCCCCN2C(=O)CC(*)C2=O)c1] | 131 |
| 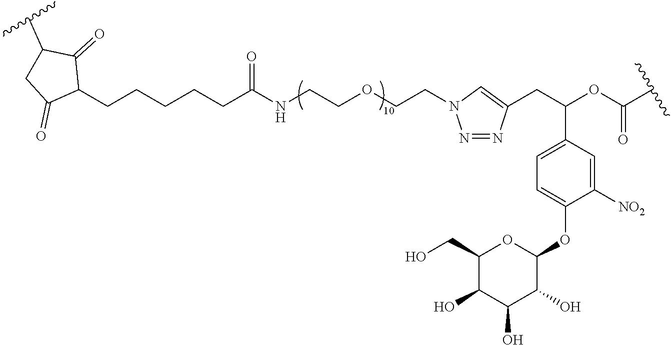 | 132 |
| 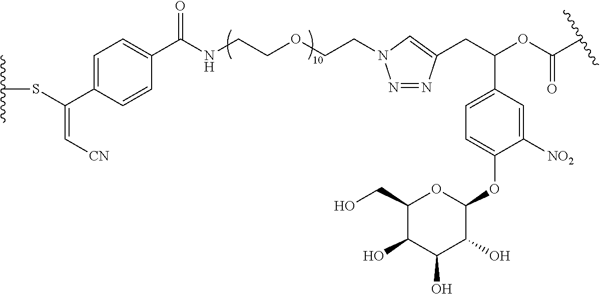 | 133 |
| 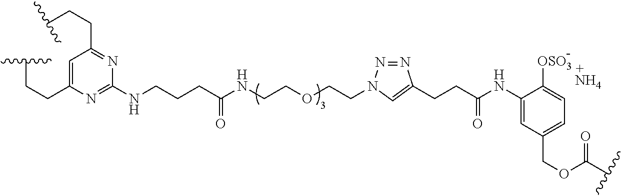 | 134 |
| 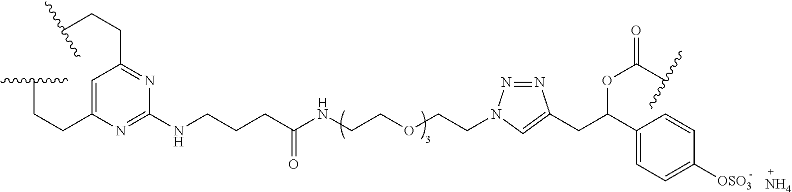 | 135 |
| 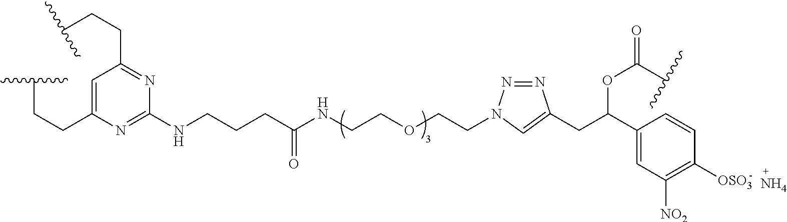 | 136 |
| 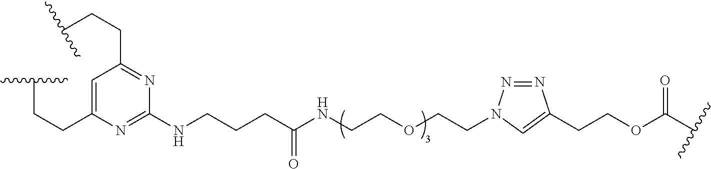 | 137 |
| 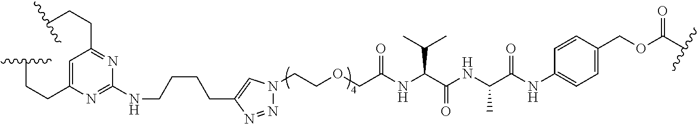 | 138 |
| 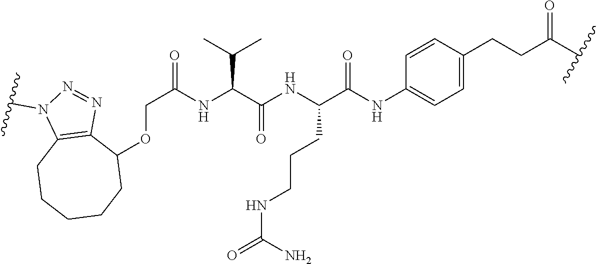 [SMILES: *C(=O)CCc1ccc(NC(=O)[C@H](CCCNC(N)=O)NC(=O)[C@@H](NC(=O)COC2CCCCCc3c2nnn3*)C(C)C)cc1] | 139 |
| 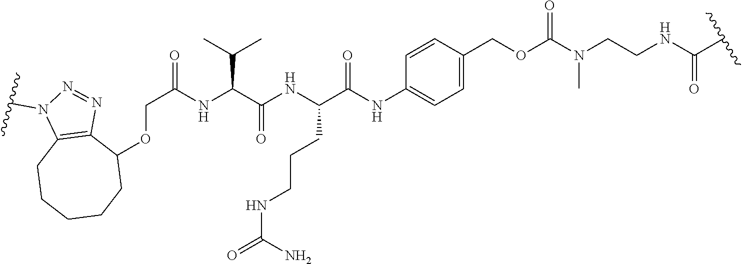 [SMILES: *C(=O)NCCN(C)C(=O)OCc1ccc(NC(=O)[C@H](CCCNC(N)=O)NC(=O)[C@@H](NC(=O)COC2CCCCCc3c2nnn3*)C(C)C)cc1] | 140 |
| 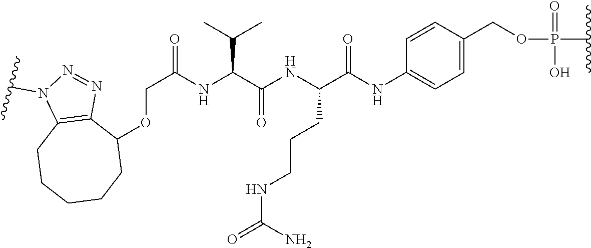 [SMILES: *n1nnc2c1CCCCCC2OCC(=O)N[C@H](C(=O)N[C@@H](CCCNC(N)=O)C(=O)Nc1ccc(COP(*)(=O)O)cc1)C(C)C] | 141 |
| 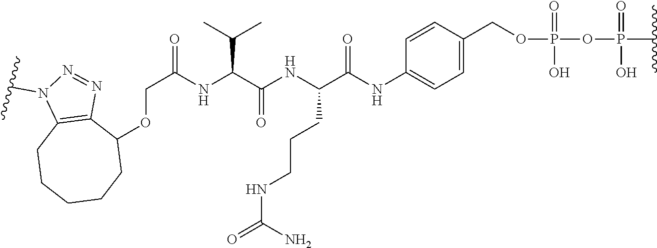 [SMILES: *n1nnc2c1CCCCCC2OCC(=O)N[C@H](C(=O)N[C@@H](CCCNC(N)=O)C(=O)Nc1ccc(COP(=O)(O)OP(*)(=O)O)cc1)C(C)C] | 142 |
| 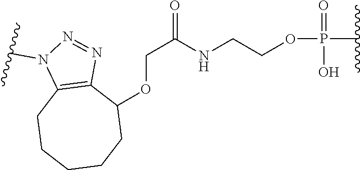 [SMILES: *n1nnc2c1CCCCCC2OCC(=O)NCCOP(*)(=O)O] | 143 |
| 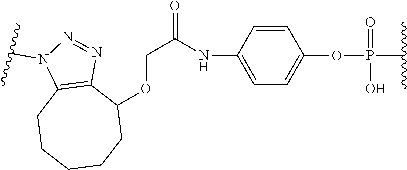 [SMILES: *n1nnc2c1CCCCCC2OCC(=O)Nc1ccc(OP(*)(=O)O)cc1] | 144 |
| 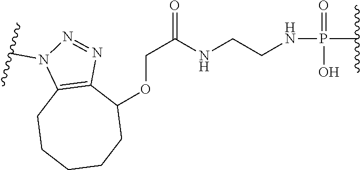 [SMILES: *n1nnc2c1CCCCCC2OCC(=O)NCCNP(*)(=O)O] | 145 |
| 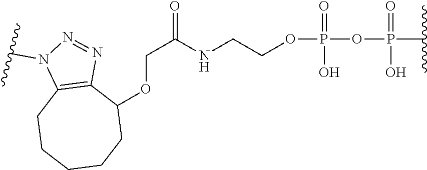 [SMILES: *n1nnc2c1CCCCCC2OCC(=O)NCCOP(=O)(O)OP(*)(=O)O] | 146 |
| 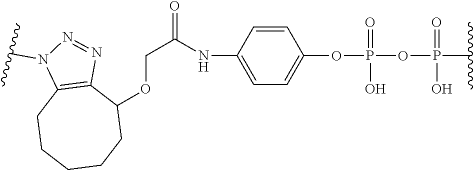 [SMILES: *n1nnc2c1CCCCCC2OCC(=O)Nc1ccc(OP(=O)(O)OP(*)(=O)O)cc1] | 147 |
| 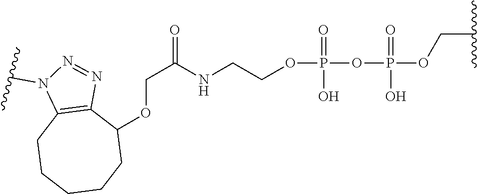 [SMILES: *COP(=O)(O)OP(=O)(O)OCCNC(=O)COC1CCCCCc2c1nnn2*] | 148 |
| 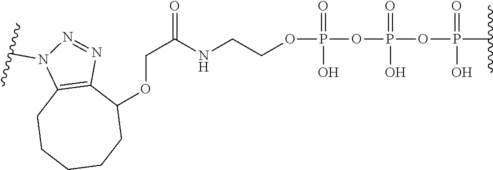 [SMILES: *n1nnc2c1CCCCCC2OCC(=O)NCCOP(=O)(O)OP(=O)(O)OP(*)(=O)O] | 149 |
| 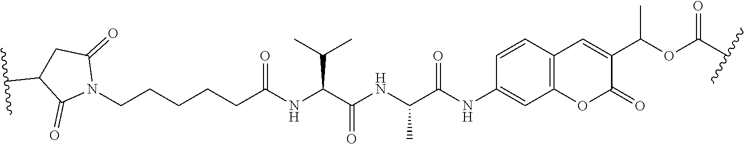 [SMILES: *C(=O)OC(C)c1cc2ccc(NC(=O)[C@H](C)NC(=O)[C@@H](NC(=O)CCCCCN3C(=O)CC(*)C3=O)C(C)C)cc2oc1=O] | 150 |
| 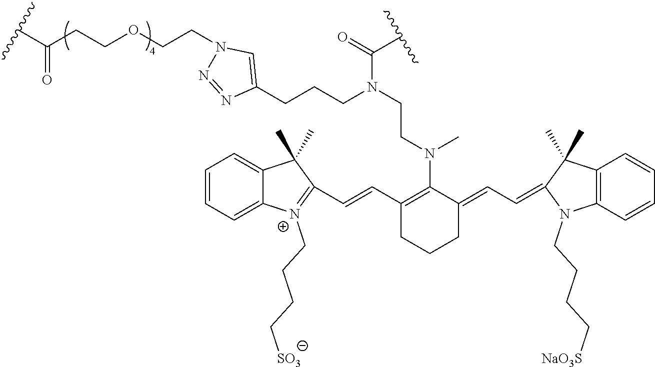 | 151 |
| 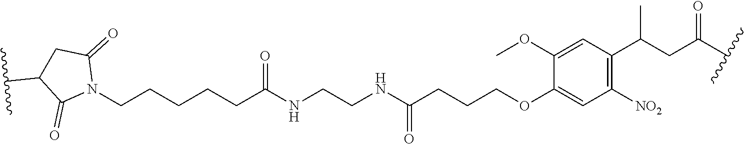 [SMILES: *C(=O)CC(C)c1cc(OC)c(OCCCC(=O)NCCNC(=O)CCCCCN2C(=O)CC(*)C2=O)cc1[N+](=O)[O-]] | 152 |
| 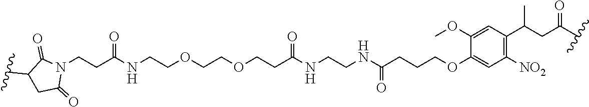 [SMILES: *C(=O)CC(C)c1cc(OC)c(OCCCC(=O)NCCNC(=O)CCOCCOCCNC(=O)CCN2C(=O)CC(*)C2=O)cc1[N+](=O)[O-]] | 153 |
| 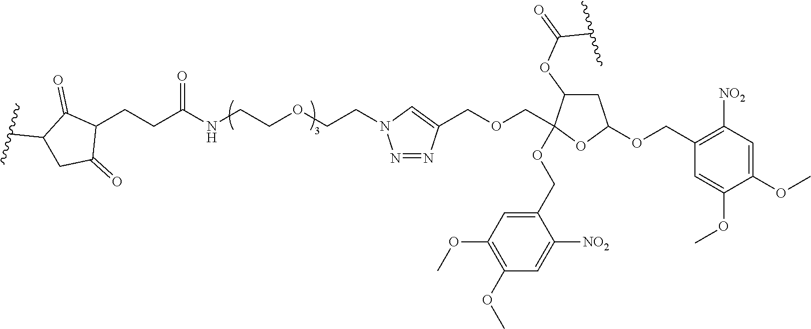 | 154 |
| 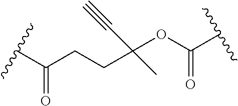 [SMILES: *C(=O)CCC(C)(C#C)OC(*)=O] | 155 |
| 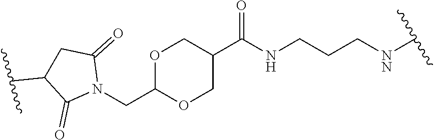 | 156 |
| 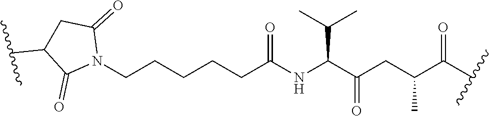 [SMILES: *C(=O)[C@H](C)CC(=O)[C@@H](NC(=O)CCCCCN1C(=O)CC(*)C1=O)C(C)C] | 157 |
| 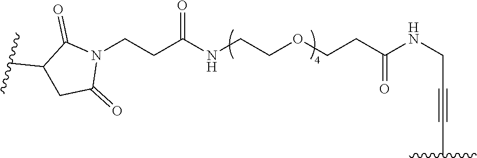 | 158 |
| 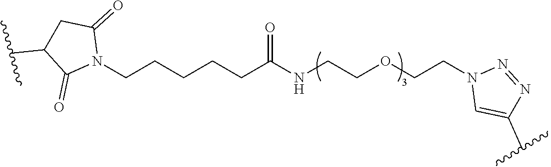 | 159 |
| 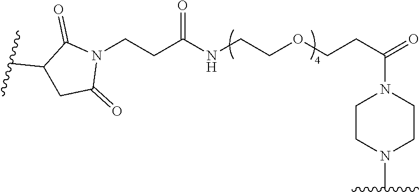 | 160 |
| 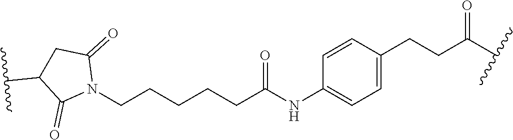 [SMILES: *C(=O)CCc1ccc(NC(=O)CCCCCN2C(=O)CC(*)C2=O)cc1] | 161 |
| 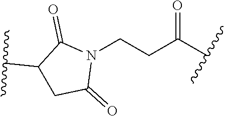 [SMILES: *C(=O)CCN1C(=O)CC(*)C1=O] | 162 |
| 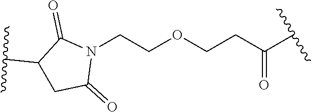 [SMILES: *C(=O)CCOCCN1C(=O)CC(*)C1=O] | 163 |
| 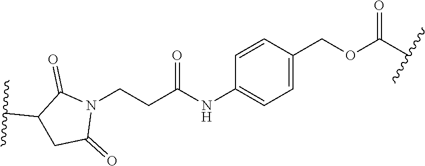 [SMILES: *C(=O)OCc1ccc(NC(=O)CCN2C(=O)CC(*)C2=O)cc1] | 164 |
| 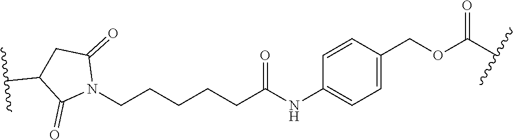 [SMILES: *C(=O)OCc1ccc(NC(=O)CCCCCN2C(=O)CC(*)C2=O)cc1] | 165 |
| 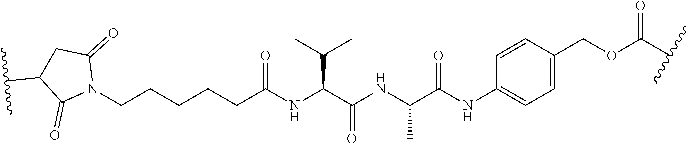 [SMILES: *C(=O)OCc1ccc(NC(=O)[C@H](C)NC(=O)[C@@H](NC(=O)CCCCCN2C(=O)CC(*)C2=O)C(C)C)cc1] | 166 |
| 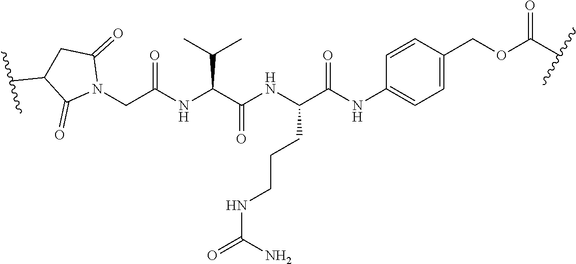 [SMILES: *C(=O)OCc1ccc(NC(=O)[C@H](CCCNC(N)=O)NC(=O)[C@@H](NC(=O)CN2C(=O)CC(*)C2=O)C(C)C)cc1] | 167 |
| 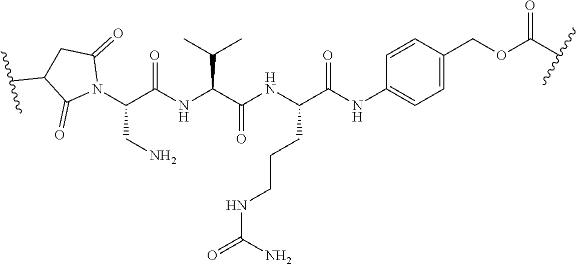 [SMILES: *C(=O)OCc1ccc(NC(=O)[C@H](CCCNC(N)=O)NC(=O)[C@@H](NC(=O)[C@H](CN)N2C(=O)CC(*)C2=O)C(C)C)cc1] | 168 |
| 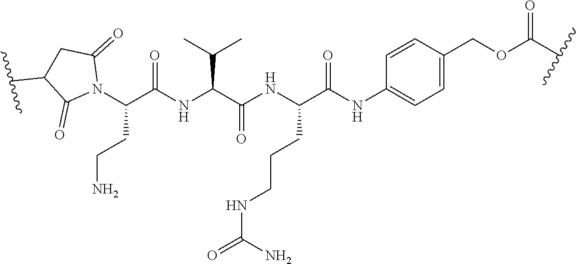 [SMILES: *C(=O)OCc1ccc(NC(=O)[C@H](CCCNC(N)=O)NC(=O)[C@@H](NC(=O)[C@H](CCN)N2C(=O)CC(*)C2=O)C(C)C)cc1] | 169 |
| 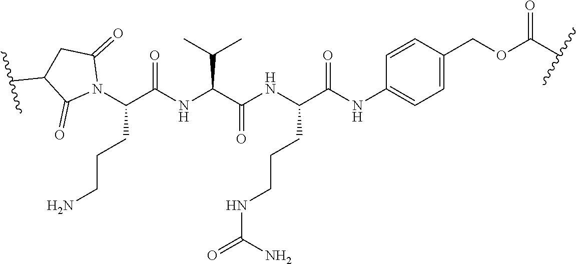 [SMILES: *C(=O)OCc1ccc(NC(=O)[C@H](CCCNC(N)=O)NC(=O)[C@@H](NC(=O)[C@H](CCCN)N2C(=O)CC(*)C2=O)C(C)C)cc1] | 170 |
| 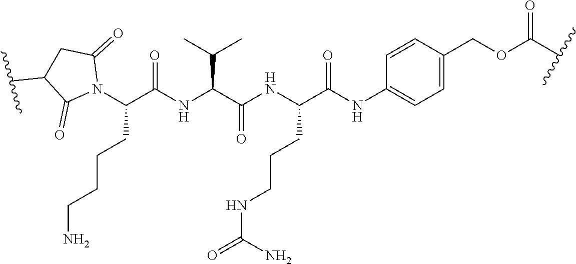 [SMILES: *C(=O)OCc1ccc(NC(=O)[C@H](CCCNC(N)=O)NC(=O)[C@@H](NC(=O)[C@H](CCCCN)N2C(=O)CC(*)C2=O)C(C)C)cc1] | 171 |
| 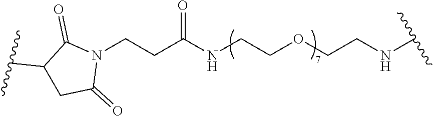 | 172 |
| 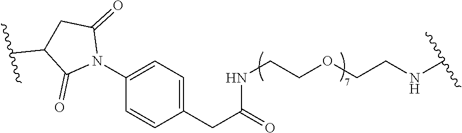 | 173 |
| 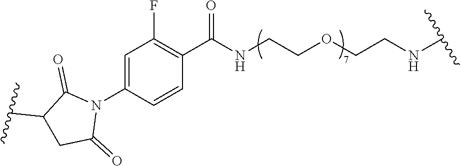 | 174 |
| 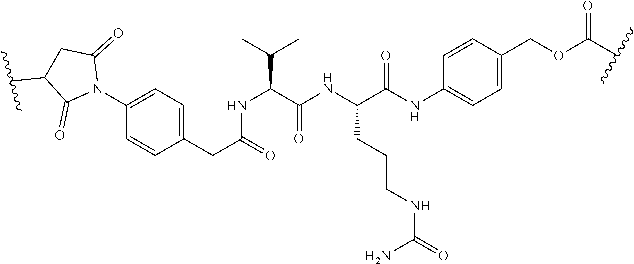 [SMILES: *C(=O)OCc1ccc(NC(=O)[C@H](CCCNC(N)=O)NC(=O)[C@@H](NC(=O)Cc2ccc(N3C(=O)CC(*)C3=O)cc2)C(C)C)cc1] | 175 |
| 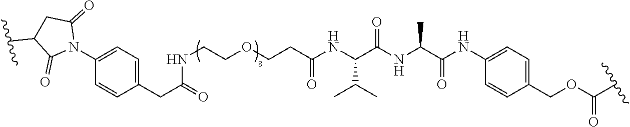 | 176 |
| 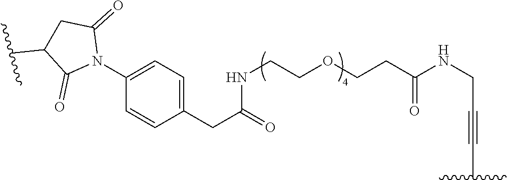 | 177 |
| 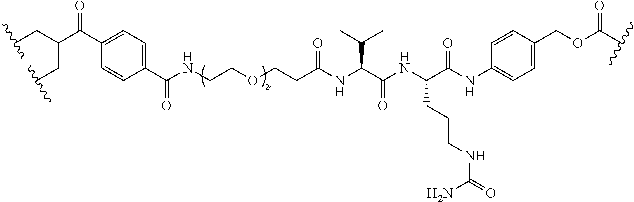 | 178 |
| 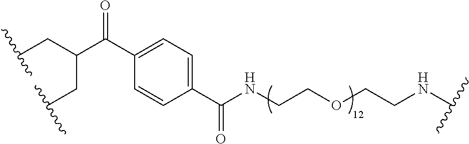 | 179 |
| 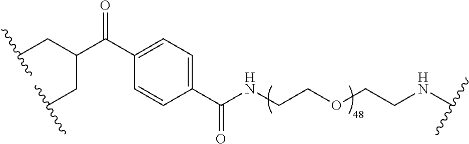 | 180 |
| 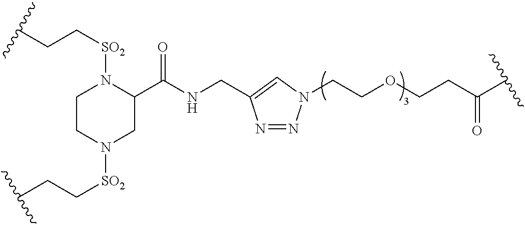 | 181 |
| 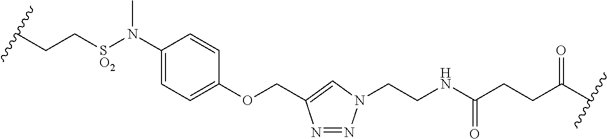 [SMILES: *CCS(=O)(=O)N(C)c1ccc(OCc2cn(CCNC(=O)CCC(*)=O)nn2)cc1] | 182 |
| 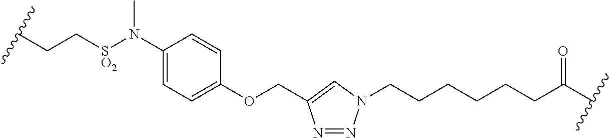 [SMILES: *CCS(=O)(=O)N(C)c1ccc(OCc2cn(CCCCCCC(*)=O)nn2)cc1] | 183 |
| 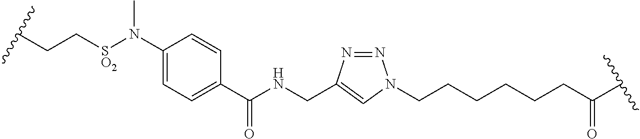 [SMILES: *CCS(=O)(=O)N(C)c1ccc(C(=O)NCc2cn(CCCCCCC(*)=O)nn2)cc1] | 184 |
| 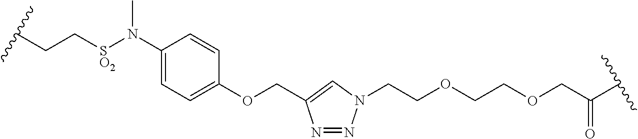 [SMILES: *CCS(=O)(=O)N(C)c1ccc(OCc2cn(CCOCCOCC(*)=O)nn2)cc1] | 185 |
| 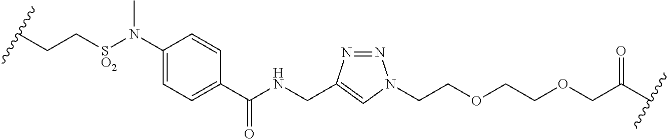 [SMILES: *CCS(=O)(=O)N(C)c1ccc(C(=O)NCc2cn(CCOCCOCC(*)=O)nn2)cc1] | 186 |
| 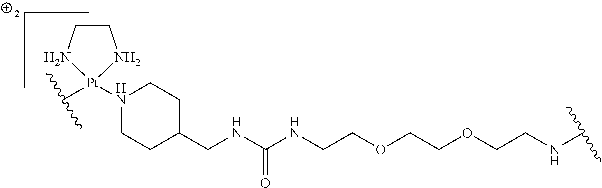 | 187 |
| 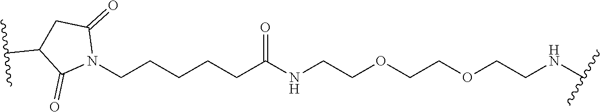 [SMILES: *NCCOCCOCCNC(=O)CCCCCN1C(=O)CC(*)C1=O] | 188 |
| 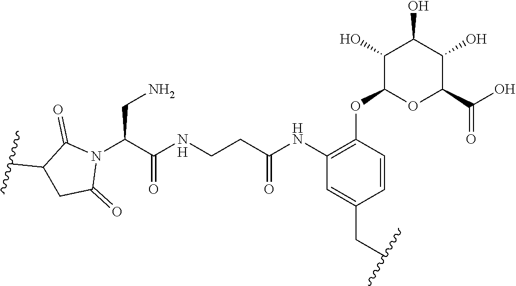 | 189 |
| 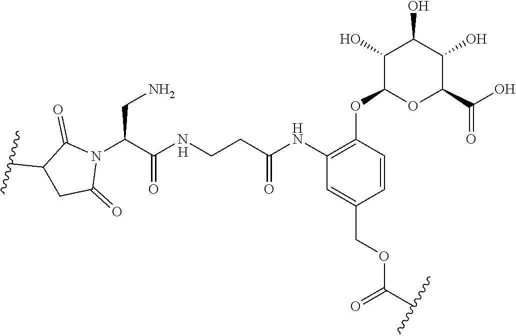 | 190 |
| 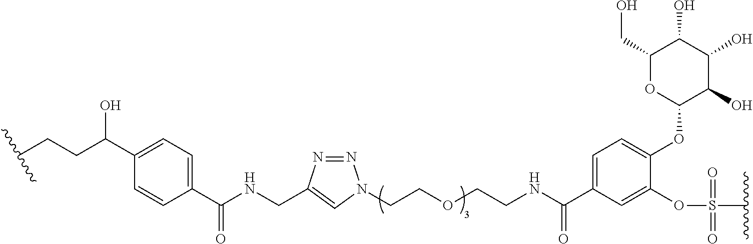 | 191 |
| 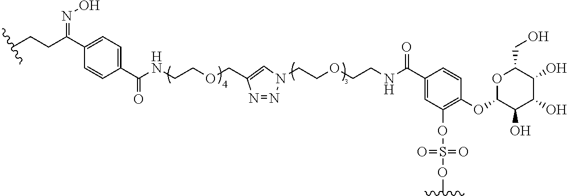 | 192 |
| 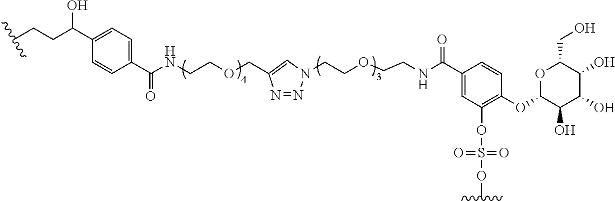 | 193 |
| 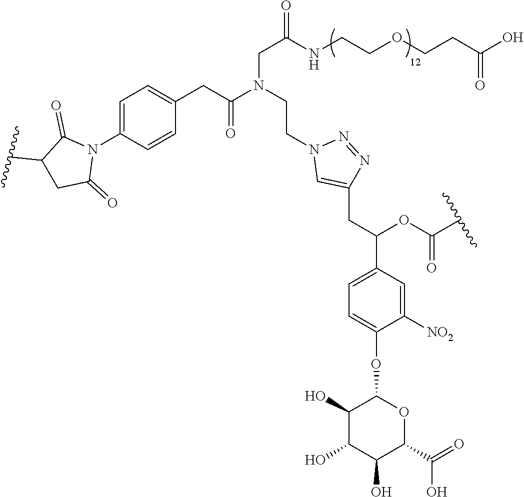 | 194 |
| 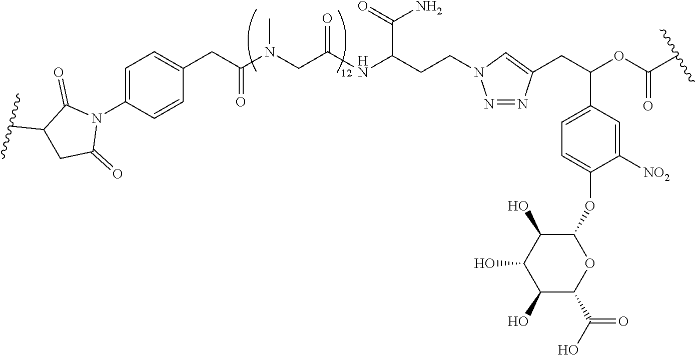 | 195 |
| 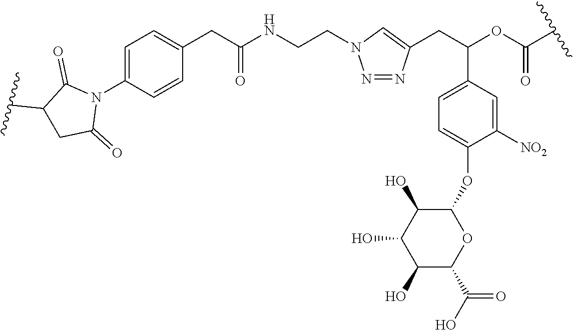 [SMILES: *C(=O)OC(Cc1cn(CCNC(=O)Cc2ccc(N3C(=O)CC(*)C3=O)cc2)nn1)c1ccc(O[C@@H]2O[C@H](C(=O)O)[C@@H](O)[C@H](O)[C@H]2O)c([N+](=O)[O-])c1] | 196 |
| 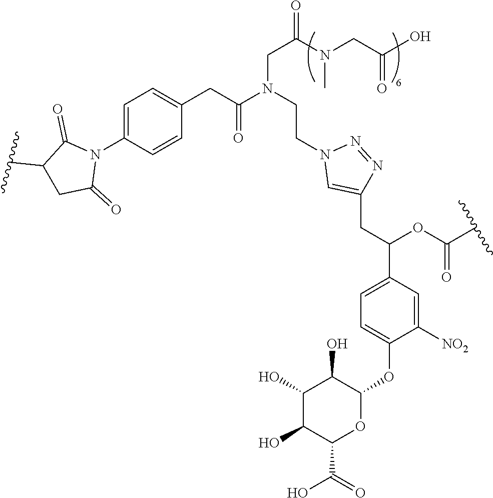 | 197 |
| 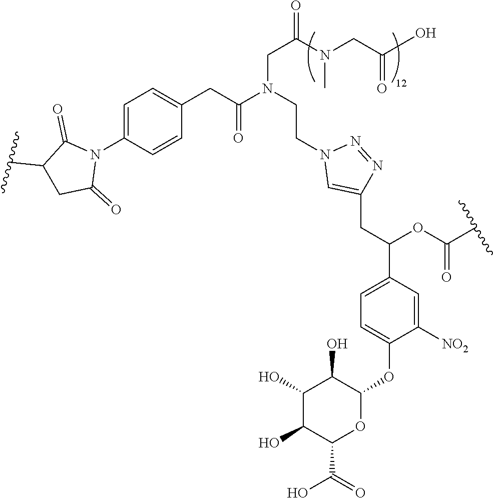 | 198 |
| 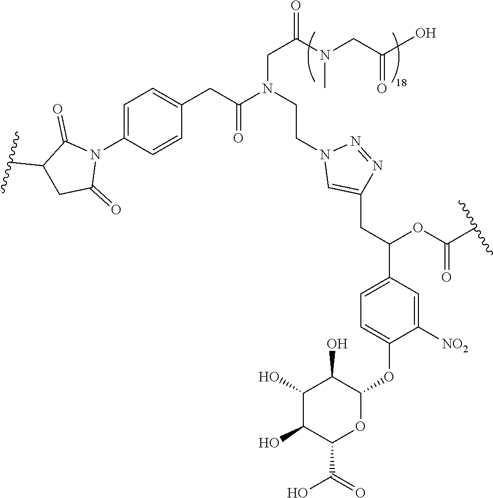 | 199 |
| 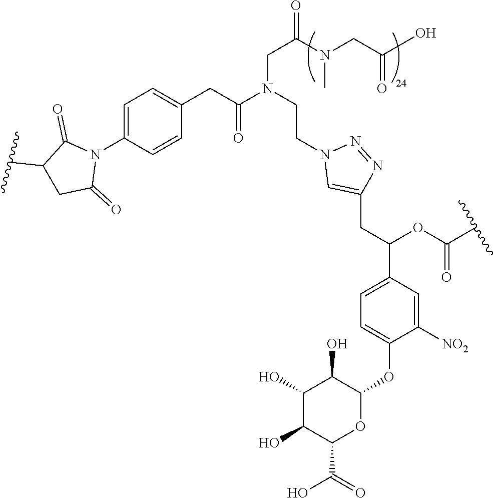 | 200 |
| 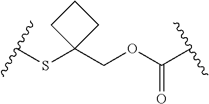 [SMILES: *SC1(COC(*)=O)CCC1] | 201 |
| 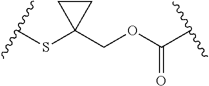 [SMILES: *SC1(COC(*)=O)CC1] | 202 |
| 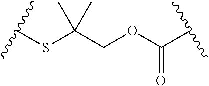 [SMILES: *SC(C)(C)COC(*)=O] | 203 |
| 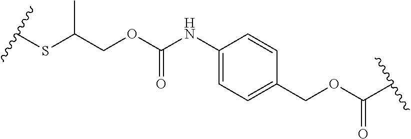 [SMILES: *SC(C)COC(=O)Nc1ccc(COC(*)=O)cc1] | 204 |
| 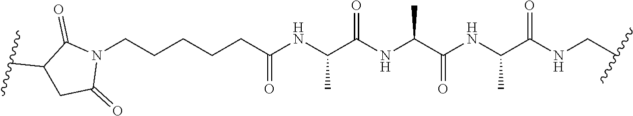 [SMILES: *CNC(=O)[C@H](C)NC(=O)[C@H](C)NC(=O)[C@H](C)NC(=O)CCCCCN1C(=O)CC(*)C1=O] | 205 |
| 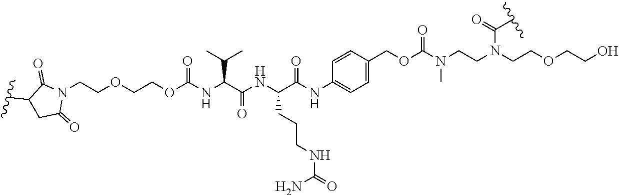 [SMILES: *C(=O)N(CCOCCO)CCN(C)C(=O)OCc1ccc(NC(=O)[C@H](CCCNC(N)=O)NC(=O)[C@@H](NC(=O)OCCOCCN2C(=O)CC(*)C2=O)C(C)C)cc1] | 206 |
| 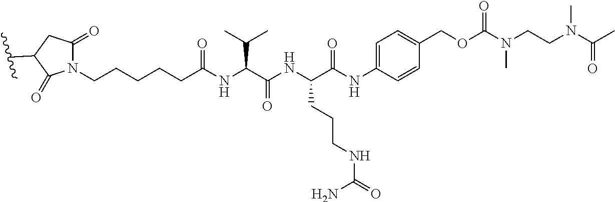 [SMILES: *C1CC(=O)N(CCCCCC(=O)N[C@H](C(=O)N[C@@H](CCCNC(N)=O)C(=O)Nc2ccc(COC(=O)N(C)CCN(C)C(C)=O)cc2)C(C)C)C1=O] | 207 |
| 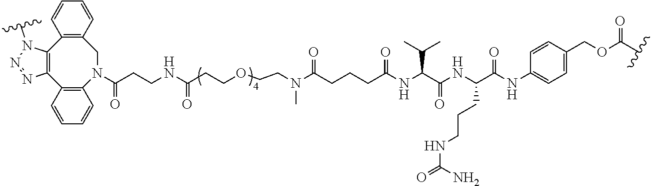 | 208 |
| 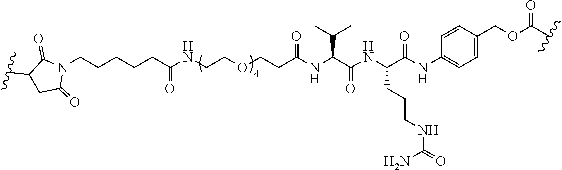 | 209 |
| 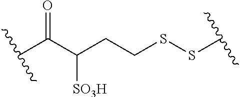 [SMILES: *SSCCC(C(*)=O)S(=O)(=O)O] | 210 |
| 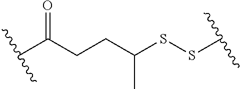 [SMILES: *SSC(C)CCC(*)=O] | 211 |
| 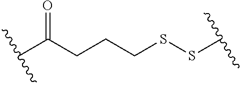 [SMILES: *SSCCCC(*)=O] | 212 |
| 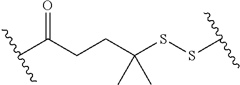 [SMILES: *SSC(C)(C)CCC(*)=O] | 213 |
| 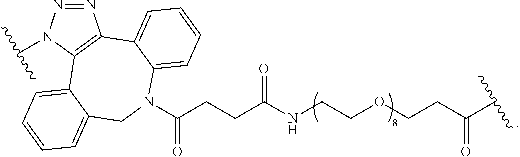 | 214 |

### D. Exemplary Linker-Payloads

In some embodiments, the present disclosure provides pharmaceutical compositions comprising an antibody-drug conjugate (ADC) having the formula A-(L-Pᵣ)_{q}, where: A is a 3E10 antibody or antigen-binding fragment thereof as described herein (e.g., as described in the section titled 3E10 Antibodies and Antigen-Binding Fragments), L is a linker, P is a payload moiety, r is an integer from 1 to 4 and q is an integer from 1 to 16.

In some embodiments, L is a cathepsin-L cleavable linker and P is a topoisomerase inhibitor. In some embodiments, the cathepsin-L cleavable linker comprises a-Val-Cit-dipeptide and P is a topoisomerase inhibitor. In some embodiments, the cathepsin-L cleavable linker comprises a -Phe-Gln- dipeptide and P is a topoisomerase inhibitor. In embodiments, the cathepsin-L cleavable linker comprises a -Val-Gln- dipeptide and P is a topoisomerase inhibitor. In embodiments, the cathepsin-L cleavable linker comprises a -Leu-Gln- dipeptide and P is a topoisomerase inhibitor. In embodiments, the cathepsin-L comprises cleavable linker comprises a -Tyr-Met- dipeptide and P is a topoisomerase inhibitor. In embodiments, the cathepsin-L comprises cleavable linker comprises a -Phe-Arg- dipeptide and P is a topoisomerase inhibitor. In embodiments, the cathepsin-L comprises cleavable linker comprises a -Phe-Gly- dipeptide and P is a topoisomerase inhibitor. In embodiments, the cathepsin-L comprises cleavable linker comprises a -Trp-Thr- dipeptide and P is a topoisomerase inhibitor. In embodiments, the cathepsin-L comprises cleavable linker comprises a -Tyr-Gly- dipeptide and P is a topoisomerase inhibitor. In embodiments, the cathepsin-L comprises cleavable linker comprises a -Phe-Thr- dipeptide and P is a topoisomerase inhibitor. In embodiments, the cathepsin-L comprises cleavable linker comprises a -Val-Gly- dipeptide and P is a topoisomerase inhibitor. In some embodiments, the cathepsin-L cleavable linker is attached to the 3E10 antibody or antigen-binding fragment thereof at a lysine residue. In some embodiments, the cathepsin-L cleavable linker is attached to the 3E10 antibody or antigen-binding fragment thereof at a cysteine residue. In some embodiments, the cathepsin-L cleavable linker is attached to the 3E10 antibody or antigen-binding fragment thereof at a glutamine residue.

In some embodiments of an ADC with a cathepsin-L cleavable linker and a topoisomerase inhibitor, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker and a topoisomerase inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cathepsin-L cleavable linker and a topoisomerase inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cathepsin-L cleavable linker and P is PNU-159682. In some embodiments, the cathepsin-L cleavable linker comprises a -Val-Cit- dipeptide and P is PNU-159682. In some embodiments, the cathepsin-L cleavable linker comprises a -Phe-Gln-dipeptide and P is PNU-159682. In embodiments, the cathepsin-L cleavable linker comprises a -Val-Gln-dipeptide and P is PNU-159682. In embodiments, the cathepsin-L cleavable linker comprises a -Leu-Gln- dipeptide and P is PNU-159682. In embodiments, the cathepsin-L comprises cleavable linker comprises a -Tyr-Met- dipeptide and P is PNU-159682. In embodiments, the cathepsin-L comprises cleavable linker comprises a -Phe-Arg- dipeptide and P is PNU-159682. In embodiments, the cathepsin-L comprises cleavable linker comprises a -Phe-Gly-dipeptide and P is PNU-159682. In embodiments, the cathepsin-L comprises cleavable linker comprises a -Trp-Thr- dipeptide and P is PNU-159682. In embodiments, the cathepsin-L comprises cleavable linker comprises a -Tyr-Gly- dipeptide and P is PNU-159682. In embodiments, the cathepsin-L comprises cleavable linker comprises a -Phe-Thr- dipeptide and P is PNU-159682. In embodiments, the cathepsin-L comprises cleavable linker comprises a -Val-Gly-dipeptide and P is PNU-159682. In some embodiments, the cathepsin-L cleavable linker is attached to the 3E10 antibody or antigen-binding fragment thereof at a lysine residue. In some embodiments, the cathepsin-L cleavable linker is attached to the 3E10 antibody or antigen-binding fragment thereof at a cysteine residue. In some embodiments, the cathepsin-L cleavable linker is attached to the 3E10 antibody or antigen-binding fragment thereof at a glutamine residue.

In some embodiments of an ADC with a cathepsin-L cleavable linker and PNU-159682, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker and PNU-159682 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cathepsin-L cleavable linker and PNU-159682, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cathepsin-L cleavable linker and P is SN-38. In some embodiments, the cathepsin-L cleavable linker comprises a -Val-Cit- dipeptide and P is SN-38. In some embodiments, the cathepsin-L cleavable linker comprises a -Phe-Gln- dipeptide and P is SN-38. In embodiments, the cathepsin-L cleavable linker comprises a -Val-Gln- dipeptide and P is SN-38. In embodiments, the cathepsin-L cleavable linker comprises a -Leu-Gln- dipeptide and P is SN-38. In embodiments, the cathepsin-L comprises cleavable linker comprises a -Tyr-Met-dipeptide and P is SN-38. In embodiments, the cathepsin-L comprises cleavable linker comprises a -Phe-Arg- dipeptide and P is SN-38. In embodiments, the cathepsin-L comprises cleavable linker comprises a -Phe-Gly- dipeptide and P is SN-38. In embodiments, the cathepsin-L comprises cleavable linker comprises a -Trp-Thr- dipeptide and P is SN-38. In embodiments, the cathepsin-L comprises cleavable linker comprises a -Tyr-Gly- dipeptide and P is SN-38. In embodiments, the cathepsin-L comprises cleavable linker comprises a -Phe-Thr- dipeptide and P is SN-38. In embodiments, the cathepsin-L comprises cleavable linker comprises a -Val-Gly-dipeptide and P is SN-38. In some embodiments, the cathepsin-L cleavable linker is attached to the 3E10 antibody or antigen-binding fragment thereof at a lysine residue. In some embodiments, the cathepsin-L cleavable linker is attached to the 3E10 antibody or antigen-binding fragment thereof at a cysteine residue. In some embodiments, the cathepsin-L cleavable linker is attached to the 3E10 antibody or antigen-binding fragment thereof at a glutamine residue.

In some embodiments of an ADC with a cathepsin-L cleavable linker and SN-38, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker and SN-38 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cathepsin-L cleavable linker and SN-38, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cathepsin-L cleavable linker and P is a tubulin inhibitor. In some embodiments, the cathepsin-L cleavable linker comprises a -Val-Cit- dipeptide and P is a tubulin inhibitor. In some embodiments, the cathepsin-L cleavable linker comprises a -Phe-Gln-dipeptide and P is a tubulin inhibitor. In embodiments, the cathepsin-L cleavable linker comprises a -Val-Gln- dipeptide and P is a tubulin inhibitor. In embodiments, the cathepsin-L cleavable linker comprises a -Leu-Gln- dipeptide and P is a tubulin inhibitor. In embodiments, the cathepsin-L comprises cleavable linker comprises a -Tyr-Met- dipeptide and P is a tubulin inhibitor. In embodiments, the cathepsin-L comprises cleavable linker comprises a -Phe-Arg-dipeptide and P is a tubulin inhibitor. In embodiments, the cathepsin-L comprises cleavable linker comprises a -Phe-Gly- dipeptide and P is a tubulin inhibitor. In embodiments, the cathepsin-L comprises cleavable linker comprises a -Trp-Thr- dipeptide and P is a tubulin inhibitor. In embodiments, the cathepsin-L comprises cleavable linker comprises a -Tyr-Gly-dipeptide and P is a tubulin inhibitor. In embodiments, the cathepsin-L comprises cleavable linker comprises a -Phe-Thr- dipeptide and P is a tubulin inhibitor. In embodiments, the cathepsin-L comprises cleavable linker comprises a -Val-Gly- dipeptide and P is a tubulin inhibitor. In some embodiments, the cathepsin-L cleavable linker is attached to the 3E10 antibody or antigen-binding fragment thereof at a lysine residue. In some embodiments, the cathepsin-L cleavable linker is attached to the 3E10 antibody or antigen-binding fragment thereof at a cysteine residue. In some embodiments, the cathepsin-L cleavable linker is attached to the 3E10 antibody or antigen-binding fragment thereof at a glutamine residue.

In some embodiments of an ADC with a cathepsin-L cleavable linker and a tubulin inhibitor, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker and a tubulin inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cathepsin-L cleavable linker and a tubulin inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cathepsin-L cleavable linker and P is MMAE. In some embodiments, the cathepsin-L cleavable linker comprises a -Val-Cit- dipeptide and P is MMAE. In some embodiments, the cathepsin-L cleavable linker comprises a -Phe-Gln- dipeptide and P is MMAE. In embodiments, the cathepsin-L cleavable linker comprises a -Val-Gln- dipeptide and P is MMAE. In embodiments, the cathepsin-L cleavable linker comprises a -Leu-Gln- dipeptide and P is MMAE. In embodiments, the cathepsin-L comprises cleavable linker comprises a -Tyr-Met-dipeptide and P is MMAE. In embodiments, the cathepsin-L comprises cleavable linker comprises a -Phe-Arg- dipeptide and P is MMAE. In embodiments, the cathepsin-L comprises cleavable linker comprises a -Phe-Gly- dipeptide and P is MMAE. In embodiments, the cathepsin-L comprises cleavable linker comprises a -Trp-Thr- dipeptide and P is MMAE. In embodiments, the cathepsin-L comprises cleavable linker comprises a -Tyr-Gly- dipeptide and P is MMAE. In embodiments, the cathepsin-L comprises cleavable linker comprises a -Phe-Thr-dipeptide and P is MMAE. In embodiments, the cathepsin-L comprises cleavable linker comprises a -Val-Gly- dipeptide and P is MMAE. In some embodiments, the cathepsin-L cleavable linker is attached to the 3E10 antibody or antigen-binding fragment thereof at a lysine residue. In some embodiments, the cathepsin-L cleavable linker is attached to the 3E10 antibody or antigen-binding fragment thereof at a cysteine residue. In some embodiments, the cathepsin-L cleavable linker is attached to the 3E10 antibody or antigen-binding fragment thereof at a glutamine residue.

In some embodiments of an ADC with a cathepsin-L cleavable linker and MMAE, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker and MMAE are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cathepsin-L cleavable linker and MMAE, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Phe-Gln dipeptide and P is a topoisomerase inhibitor. In some embodiments, the cleavable linker comprising a -Phe-Gln dipeptide is attached to the 3E10 antibody or antigen-binding fragment thereof at a lysine residue. In some embodiments, the cleavable linker comprising a -Phe-Gln dipeptide is attached to the 3E10 antibody or antigen-binding fragment thereof at a cysteine residue. In some embodiments, the cleavable linker comprising a -Phe-Gln dipeptide is attached to the 3E10 antibody or antigen-binding fragment thereof at a glutamine residue.

In some embodiments of an ADC with a cleavable linker comprising a Phe-Gln dipeptide and a topoisomerase inhibitor, the cleavable linker comprising a -Phe-Gln dipeptide is an un-branched linker. In some embodiments, the cleavable linker comprising a -Phe-Gln dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a Phe-Gln dipeptide and a topoisomerase inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a Phe-Gln dipeptide and a topoisomerase inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Phe-Gln dipeptide and P is PNU-159682. In some embodiments, the cleavable linker comprising a -Phe-Gln dipeptide is attached to the 3E10 antibody or antigen-binding fragment thereof at a lysine residue. In some embodiments, the cleavable linker comprising a -Phe-Gln dipeptide is attached to the 3E10 antibody or antigen-binding fragment thereof at a cysteine residue. In some embodiments, the cleavable linker comprising a -Phe-Gln dipeptide is attached to the 3E10 antibody or antigen-binding fragment thereof at a glutamine residue.

In some embodiments of an ADC with a cleavable linker comprising a -Phe-Gln dipeptide and PNU-159682, the cleavable linker comprising a Phe-Gln dipeptide is an un-branched linker. In some embodiments, the cleavable linker comprising a -Phe-Gln dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Phe-Gln dipeptide and PNU-159682 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Phe-Gln dipeptide and PNU-159682, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Phe-Gin dipeptide and P is SN-38. In some embodiments, the cleavable linker comprising a -Phe-Gin dipeptide is attached to the 3E10 antibody or antigen-binding fragment thereof at a lysine residue. In some embodiments, the cleavable linker comprising a -Phe-Gln dipeptide is attached to the 3E10 antibody or antigen-binding fragment thereof at a cysteine residue. In some embodiments, the cleavable linker comprising a -Phe-Gln dipeptide is attached to the 3E10 antibody or antigen-binding fragment thereof at a glutamine residue.

In some embodiments of an ADC with a cleavable linker comprising a -Phe-Gln dipeptide and SN-38, the cleavable linker comprising a -Phe-Gln dipeptide is an un-branched linker. In some embodiments, the cleavable linker comprising a -Phe-Gln dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a Phe-Gln dipeptide and SN-38 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Phe-Gln dipeptide and SN-38, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Phe-Gln dipeptide and P is a tubulin inhibitor. In some embodiments, the cleavable linker comprising a -Phe-Gln dipeptide is attached to the 3E10 antibody or antigen-binding fragment thereof at a lysine residue. In some embodiments, the cleavable linker comprising a -Phe-Gln dipeptide is attached to the 3E10 antibody or antigen-binding fragment thereof at a cysteine residue. In some embodiments, the cleavable linker comprising a -Phe-Gln dipeptide is attached to the 3E10 antibody or antigen-binding fragment thereof at a glutamine residue.

In some embodiments of an ADC with a cleavable linker comprising a -Phe-Gln dipeptide and a tubulin inhibitor, the cleavable linker comprising a -Phe-Gln dipeptide is an un-branched linker. In some embodiments, the cleavable linker comprising a -Phe-Gln dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Phe-Gln dipeptide and a tubulin inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Phe-Gln dipeptide and a tubulin inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Phe-Gln dipeptide and P is MMAE. In some embodiments, the cleavable linker comprising a -Phe-Gln dipeptide is attached to the 3E10 antibody or antigen-binding fragment thereof at a lysine residue. In some embodiments, the cleavable linker comprising a -Phe-Gln dipeptide is attached to the 3E10 antibody or antigen-binding fragment thereof at a cysteine residue. In some embodiments, the cleavable linker comprising a -Phe-Gln dipeptide is attached to the 3E10 antibody or antigen-binding fragment thereof at a glutamine residue.

In some embodiments of an ADC with a cleavable linker comprising a -Phe-Gln dipeptide and MMAE, the cleavable linker comprising a -Phe-Gln dipeptide is an un-branched linker. In some embodiments, the cleavable linker comprising a -Phe-Gln dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Phe-Gln dipeptide and MMAE are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Phe-Gin dipeptide and MMAE, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Val-Gin dipeptide and P is a topoisomerase inhibitor. In some embodiments, the cleavable linker comprising a -Val-Gln dipeptide is attached to the 3E10 antibody or antigen-binding fragment thereof at a lysine residue. In some embodiments, the cleavable linker comprising a -Val-Gln dipeptide is attached to the 3E10 antibody or antigen-binding fragment thereof at a cysteine residue. In some embodiments, the cleavable linker comprising a -Val-Gin dipeptide is attached to the 3E10 antibody or antigen-binding fragment thereof at a glutamine residue.

In some embodiments of an ADC with a cleavable linker comprising a -Val-Gln dipeptide and a topoisomerase inhibitor, the cleavable linker comprising a -Val-Gin dipeptide is an un-branched linker. In some embodiments, the cleavable linker comprising a -Val-Gln dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Val-Gln dipeptide and a topoisomerase inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Val-Gln dipeptide and a topoisomerase inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Val-Gin dipeptide and P is PNU-159682. In some embodiments, the cleavable linker comprising a -Val-Gln dipeptide is attached to the 3E10 antibody or antigen-binding fragment thereof at a lysine residue. In some embodiments, the cleavable linker comprising a -Val-Gin dipeptide is attached to the 3E10 antibody or antigen-binding fragment thereof at a cysteine residue. In some embodiments, the cleavable linker comprising a -Val-Gln dipeptide is attached to the 3E10 antibody or antigen-binding fragment thereof at a glutamine residue.

In some embodiments of an ADC with a cleavable linker comprising a -Val-Gln dipeptide and PNU-159682, the cleavable linker comprising a -Val-Gln dipeptide is an un-branched linker. In some embodiments, the cleavable linker comprising a -Val-Gin dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Val-Gln dipeptide and PNU-159682 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable 1% linker comprising a -Val-Gln dipeptide and PNU-159682, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Val-Gn dipeptide and P is SN-38. In some embodiments, the cleavable linker comprising a -Val-Gin dipeptide is attached to the 3E10 antibody or antigen-binding fragment thereof at a lysine residue. In some embodiments, the cleavable linker comprising a -Val-Gin dipeptide is attached to the 3E10 antibody or antigen-binding fragment thereof at a cysteine residue. In some embodiments, the cleavable linker comprising a -Val-Gln dipeptide is attached to the 3E10 antibody or antigen-binding fragment thereof at a glutamine residue.

In some embodiments of an ADC with a cleavable linker comprising a -Val-Gln dipeptide and SN-38, the cleavable linker comprising a -Val-Gln dipeptide is an un-branched linker. In some embodiments, the cleavable linker comprising a -Val-Gln dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Val-Gln dipeptide and SN-38 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Val-Gln dipeptide and SN-38, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Val-Gin dipeptide and P is a tubulin inhibitor. In some embodiments, the cleavable linker comprising a -Val-Gln dipeptide is attached to the 3E10 antibody or antigen-binding fragment thereof at a lysine residue. In some embodiments, the cleavable linker comprising a -Val-Gln dipeptide is attached to the 3E10 antibody or antigen-binding fragment thereof at a cysteine residue. In some embodiments, the cleavable linker comprising a -Val-Gin dipeptide is attached to the 3E10 antibody or antigen-binding fragment thereof at a glutamine residue.

In some embodiments of an ADC with a cleavable linker comprising a -Val-Gln dipeptide and a tubulin inhibitor, the cleavable linker comprising a -Val-Gin dipeptide is an un-branched linker. In some embodiments, the cleavable linker comprising a -Val-Gin dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Val-Gln dipeptide and a tubulin inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Val-Gln dipeptide and a tubulin inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Val-Gin dipeptide and P is MMAE. In some embodiments, the cleavable linker comprising a -Val-Gln dipeptide is attached to the 3E10 antibody or antigen-binding fragment thereof at a lysine residue. In some embodiments, the cleavable linker comprising a -Val-Gin dipeptide is attached to the 3E10 antibody or antigen-binding fragment thereof at a cysteine residue. In some embodiments, the cleavable linker comprising a -Val-Gln dipeptide is attached to the 3E10 antibody or antigen-binding fragment thereof at a glutamine residue.

In some embodiments of an ADC with a cleavable linker comprising a -Val-Gln dipeptide and MMAE, the cleavable linker comprising a -Val-Gin dipeptide is an un-branched linker. In some embodiments, the cleavable linker comprising a -Val-Gln dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Val-Gln dipeptide and MMAE are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Val-Gln dipeptide and MMAE, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a Cleavable linker comprising a -Leu-Gln- dipeptide and P is a topoisomerase inhibitor. In some embodiments, the Cleavable linker comprising a -Leu-Gln-dipeptide is attached to the 3E10 antibody or antigen-binding fragment thereof at a lysine residue. In some embodiments, the Cleavable linker comprising a -Leu-Gln- dipeptide is attached to the 3E10 antibody or antigen-binding fragment thereof at a cysteine residue. In some embodiments, the Cleavable linker comprising a -Leu-Gln- dipeptide is attached to the 3E10 antibody or antigen-binding fragment thereof at a glutamine residue.

In some embodiments of an ADC with a Cleavable linker comprising a -Leu-Gln-dipeptide and a topoisomerase inhibitor, the Cleavable linker comprising a -Leu-Gln- dipeptide is an un-branched linker. In some embodiments, the Cleavable linker comprising a -Leu-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a Cleavable linker comprising a -Leu-Gln- dipeptide and a topoisomerase inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a Cleavable linker comprising a -Leu-Gln- dipeptide and a topoisomerase inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a Cleavable linker comprising a -Leu-Gln- dipeptide and P is PNU-159682. In some embodiments, the Cleavable linker comprising a -Leu-Gln- dipeptide is attached to the 3E10 antibody or antigen-binding fragment thereof at a lysine residue. In some embodiments, the Cleavable linker comprising a -Leu-Gln- dipeptide is attached to the 3E10 antibody or antigen-binding fragment thereof at a cysteine residue. In some embodiments, the Cleavable linker comprising a -Leu-Gln- dipeptide is attached to the 3E10 antibody or antigen-binding fragment thereof at a glutamine residue.

In some embodiments of an ADC with a Cleavable linker comprising a -Leu-Gln-dipeptide and PNU-159682, the Cleavable linker comprising a -Leu-Gln- dipeptide is an un-branched linker. In some embodiments, the Cleavable linker comprising a -Leu-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a Cleavable linker comprising a -Leu-Gln- dipeptide and PNU-159682 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a Cleavable linker comprising a -Leu-Gln- dipeptide and PNU-159682, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Leu-Gln- dipeptide and P is SN-38. In some embodiments, the cleavable linker comprising a -Leu-Gln- dipeptide is attached to the 3E10 antibody or antigen-binding fragment thereof at a lysine residue. In some embodiments, the cleavable linker comprising a -Leu-Gln- dipeptide is attached to the 3E10 antibody or antigen-binding fragment thereof at a cysteine residue. In some embodiments, the cleavable linker comprising a -Leu-Gln- dipeptide is attached to the 3E10 antibody or antigen-binding fragment thereof at a glutamine residue.

In some embodiments of an ADC with a cleavable linker comprising a -Leu-Gln-dipeptide and SN-38, the cleavable linker comprising a -Leu-Gln- dipeptide is an un-branched linker. In some embodiments, the cleavable linker comprising a -Leu-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Leu-Gln- dipeptide and SN-38 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Leu-Gln- dipeptide and SN-38, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Leu-Gln- dipeptide and P is a tubulin inhibitor. In some embodiments, the cleavable linker comprising a -Leu-Gln-dipeptide is attached to the 3E10 antibody or antigen-binding fragment thereof at a lysine residue. In some embodiments, the cleavable linker comprising a -Leu-Gln- dipeptide is attached to the 3E10 antibody or antigen-binding fragment thereof at a cysteine residue. In some embodiments, the cleavable linker comprising a -Leu-Gln- dipeptide is attached to the 3E10 antibody or antigen-binding fragment thereof at a glutamine residue.

In some embodiments of an ADC with a cleavable linker comprising a -Leu-Gln-dipeptide and a tubulin inhibitor, the cleavable linker comprising a -Leu-Gln- dipeptide is an un-branched linker. In some embodiments, the cleavable linker comprising a -Leu-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Leu-Gln- dipeptide and a tubulin inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Leu-Gln- dipeptide and a tubulin inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Leu-Gln- dipeptide and P is MMAE. In some embodiments, the cleavable linker comprising a -Leu-Gln- dipeptide is attached to the 3E10 antibody or antigen-binding fragment thereof at a lysine residue. In some embodiments, the cleavable linker comprising a -Leu-Gln- dipeptide is attached to the 3E10 antibody or antigen-binding fragment thereof at a cysteine residue. In some embodiments, the cleavable linker comprising a -Leu-Gln- dipeptide is attached to the 3E10 antibody or antigen-binding fragment thereof at a glutamine residue.

In some embodiments of an ADC with a cleavable linker comprising a -Leu-Gln-dipeptide and MMAE, the cleavable linker comprising a -Leu-Gln- dipeptide is an un-branched linker. In some embodiments, the cleavable linker comprising a -Leu-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Leu-Gln- dipeptide and MMAE are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a cleavable linker comprising a -Leu-Gln- dipeptide and MMAE, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a disulfide cleavable linker and P is a topoisomerase inhibitor. In some embodiments, the disulfide cleavable linker is attached to the 3E10 antibody or antigen-binding fragment thereof at a lysine residue. In some embodiments, the disulfide cleavable linker is attached to the 3E10 antibody or antigen-binding fragment thereof at a cysteine residue. In some embodiments, the disulfide cleavable linker is attached to the 3E10 antibody or antigen-binding fragment thereof at a glutamine residue.

In some embodiments of an ADC with a disulfide cleavable linker and a topoisomerase inhibitor, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker and a topoisomerase inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a disulfide cleavable linker and a topoisomerase inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a disulfide cleavable linker and P is a PNU-159682. In some embodiments, the disulfide cleavable linker is attached to the 3E10 antibody or antigen-binding fragment thereof at a lysine residue. In some embodiments, the disulfide cleavable linker is attached to the 3E10 antibody or antigen-binding fragment thereof at a cysteine residue. In some embodiments, the disulfide cleavable linker is attached to the 3E10 antibody or antigen-binding fragment thereof at a glutamine residue.

In some embodiments of an ADC with a disulfide cleavable linker and a PNU-159682, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker and a PNU-159682 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a disulfide cleavable linker and PNU-159682, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a disulfide cleavable linker and P is SN-38. In some embodiments, the disulfide cleavable linker is attached to the 3E10 antibody or antigen-binding fragment thereof at a lysine residue. In some embodiments, the disulfide cleavable linker is attached to the 3E10 antibody or antigen-binding fragment thereof at a cysteine residue. In some embodiments, the disulfide cleavable linker is attached to the 3E10 antibody or antigen-binding fragment thereof at a glutamine residue.

In some embodiments of an ADC with a disulfide cleavable linker and SN-38, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker and SN-38 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a disulfide cleavable linker and SN-38, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a disulfide cleavable linker and P is a tubulin inhibitor. In some embodiments, the disulfide cleavable linker is attached to the 3E10 antibody or antigen-binding fragment thereof at a lysine residue. In some embodiments, the disulfide cleavable linker is attached to the 3E10 antibody or antigen-binding fragment thereof at a cysteine residue. In some embodiments, the disulfide cleavable linker is attached to the 3E10 antibody or antigen-binding fragment thereof at a glutamine residue.

In some embodiments of an ADC with a disulfide cleavable linker and a tubulin inhibitor, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker and a tubulin inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a disulfide cleavable linker and a tubulin inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a disulfide cleavable linker and P is MMAE. In some embodiments, the disulfide cleavable linker is attached to the 3E10 antibody or antigen-binding fragment thereof at a lysine residue. In some embodiments, the disulfide cleavable linker is attached to the 3E10 antibody or antigen-binding fragment thereof at a cysteine residue. In some embodiments, the disulfide cleavable linker is attached to the 3E10 antibody or antigen-binding fragment thereof at a glutamine residue.

In some embodiments of an ADC with a disulfide cleavable linker and MMAE, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker and MMAE are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a disulfide cleavable linker and MMAE, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a hindered disulfide cleavable linker and P is a topoisomerase inhibitor. In some embodiments, the hindered disulfide cleavable linker is attached to the 3E10 antibody or antigen-binding fragment thereof at a lysine residue. In some embodiments, the hindered disulfide cleavable linker is attached to the 3E10 antibody or antigen-binding fragment thereof at a cysteine residue. In some embodiments, the hindered disulfide cleavable linker is attached to the 3E10 antibody or antigen-binding fragment thereof at a glutamine residue.

In some embodiments of an ADC with a hindered disulfide cleavable linker and a topoisomerase inhibitor, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker and a topoisomerase inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a hindered disulfide cleavable linker and a topoisomerase inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a hindered disulfide cleavable linker and P is PNU-159682. In some embodiments, the hindered disulfide cleavable linker is attached to the 3E10 antibody or antigen-binding fragment thereof at a lysine residue. In some embodiments, the hindered disulfide cleavable linker is attached to the 3E10 antibody or antigen-binding fragment thereof at a cysteine residue. In some embodiments, the hindered disulfide cleavable linker is attached to the 3E10 antibody or antigen-binding fragment thereof at a glutamine residue.

In some embodiments of an ADC with a hindered disulfide cleavable linker and PNU-159682, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker and PNU-159682 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a hindered disulfide cleavable linker and PNU-159682, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a hindered disulfide cleavable linker and P is SN-38. In some embodiments, the hindered disulfide cleavable linker is attached to the 3E10 antibody or antigen-binding fragment thereof at a lysine residue. In some embodiments, the hindered disulfide cleavable linker is attached to the 3E10 antibody or antigen-binding fragment thereof at a cysteine residue. In some embodiments, the hindered disulfide cleavable linker is attached to the 3E10 antibody or antigen-binding fragment thereof at a glutamine residue.

In some embodiments of an ADC with a hindered disulfide cleavable linker and SN-38, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker and SN-38 are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a hindered disulfide cleavable linker and SN-38, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a hindered disulfide cleavable linker and P is a tubulin inhibitor. In some embodiments, the hindered disulfide cleavable linker is attached to the 3E10 antibody or antigen-binding fragment thereof at a lysine residue. In some embodiments, the hindered disulfide cleavable linker is attached to the 3E10 antibody or antigen-binding fragment thereof at a cysteine residue. In some embodiments, the hindered disulfide cleavable linker is attached to the 3E10 antibody or antigen-binding fragment thereof at a glutamine residue.

In some embodiments of an ADC with a hindered disulfide cleavable linker and a tubulin inhibitor, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker and a tubulin inhibitor are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a hindered disulfide cleavable linker and a tubulin inhibitor, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a hindered disulfide cleavable linker and P is MMAE. In some embodiments, the hindered disulfide cleavable linker is attached to the 3E10 antibody or antigen-binding fragment thereof at a lysine residue. In some embodiments, the hindered disulfide cleavable linker is attached to the 3E10 antibody or antigen-binding fragment thereof at a cysteine residue. In some embodiments, the hindered disulfide cleavable linker is attached to the 3E10 antibody or antigen-binding fragment thereof at a glutamine residue.

In some embodiments of an ADC with a hindered disulfide cleavable linker and MMAE, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker and MMAE are provided. In some such compositions, the average the drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of a 3E10 ADC with a hindered disulfide cleavable linker and MMAE, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. E. Exemplary Linker-Antibodies

In embodiments, the present disclosure provides an antibody-drug conjugate (ADC) having the formula A-(L-Pᵣ)_{q}, wherein: A is a 3E10 antibody or antigen-binding fragment or variant thereof, L is a linker, P is a payload as described herein, r is an integer from 1 to 4, and q is an integer from 1 to 16.

In some embodiments, L is a cathepsin-L cleavable linker, and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the cathepsin-L cleavable linker comprises a -Phe-Gln- dipeptide. In some embodiments, the cathepsin-L cleavable linker comprises a -Val-Gln- dipeptide. In some embodiments, the cathepsin-L cleavable linker comprises a -Leu-Gln- dipeptide. In some embodiments, the cathepsin-L cleavable linker comprises a -Tyr-Met- dipeptide. In some embodiments, the cathepsin-L cleavable linker comprises a -Phe-Arg- dipeptide. In some embodiments, the cathepsin-L cleavable linker comprises a -Phe-Gly- dipeptide. In some embodiments, the cathepsin-L comprises cleavable linker comprises a -Trp-Thr- dipeptide. In some embodiments, the cathepsin-L cleavable linker comprises a -Tyr-Gly- dipeptide. In some embodiments, the cathepsin-L cleavable linker comprises a -Phe-Thr- dipeptide. In some embodiments, the cathepsin-L cleavable linker comprises a -Val-Gly- dipeptide. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cathepsin-L cleavable linker is attached to the antibody or antigen-binding fragment thereof at a lysine residue. In some embodiments, the cathepsin-L cleavable linker is attached to the antibody or antigen-binding fragment thereof at a cysteine residue. In some embodiments, the cathepsin-L cleavable linker is attached to the antibody or antigen-binding fragment thereof at a glutamine residue.

In some embodiments of an ADC with a cathepsin-L cleavable linker and an antibody or antigen-binding fragment or variant thereof that comprises VL CDRs of SEQ ID NOs: 61, 62, and 63 and VH CDRs of SEQ ID NOs: 58, 59, and 60, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cathepsin-L cleavable linker, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the cathepsin-L cleavable linker comprises a -Phe-Gln- dipeptide. In some embodiments, the cathepsin-L cleavable linker comprises a -Val-Gln- dipeptide. In some embodiments, the cathepsin-L cleavable linker comprises a -Leu-Gln- dipeptide. In some embodiments, the cathepsin-L cleavable linker comprises a -Tyr-Met- dipeptide. In some embodiments, the cathepsin-L cleavable linker comprises a -Phe-Arg- dipeptide. In some embodiments, the cathepsin-L cleavable linker comprises a -Phe-Gly- dipeptide. In some embodiments, the cathepsin-L comprises cleavable linker comprises a -Trp-Thr- dipeptide. In some embodiments, the cathepsin-L cleavable linker comprises a -Tyr-Gly- dipeptide. In some embodiments, the cathepsin-L cleavable linker comprises a -Phe-Thr- dipeptide. In some embodiments, the cathepsin-L cleavable linker comprises a -Val-Gly- dipeptide. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cathepsin-L cleavable linker is attached to the antibody or antigen-binding fragment thereof at a lysine residue. In some embodiments, the cathepsin-L cleavable linker is attached to the antibody or antigen-binding fragment thereof at a cysteine residue. In some embodiments, the cathepsin-L cleavable linker is attached to the antibody or antigen-binding fragment thereof at a glutamine residue.

In some embodiments of an ADC with a cathepsin-L cleavable linker and an antibody or antigen-binding fragment or variant thereof that comprises VL CDRs of SEQ ID NOs: 9, 10, and 11 and VH CDRs of SEQ ID NOs: 15, 4, and 5, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cathepsin-L cleavable linker, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the cathepsin-L cleavable linker comprises a -Phe-Gln- dipeptide. In some embodiments, the cathepsin-L cleavable linker comprises a -Val-Gln- dipeptide. In some embodiments, the cathepsin-L cleavable linker comprises a -Leu-Gln- dipeptide. In some embodiments, the cathepsin-L cleavable linker comprises a -Tyr-Met- dipeptide. In some embodiments, the cathepsin-L cleavable linker comprises a -Phe-Arg- dipeptide. In some embodiments, the cathepsin-L cleavable linker comprises a -Phe-Gly- dipeptide. In some embodiments, the cathepsin-L comprises cleavable linker comprises a -Trp-Thr- dipeptide. In some embodiments, the cathepsin-L cleavable linker comprises a -Tyr-Gly- dipeptide. In some embodiments, the cathepsin-L cleavable linker comprises a -Phe-Thr- dipeptide. In some embodiments, the cathepsin-L cleavable linker comprises a -Val-Gly- dipeptide. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the cathepsin-L cleavable linker is attached to the antibody or antigen-binding fragment thereof at a lysine residue. In some embodiments, the cathepsin-L cleavable linker is attached to the antibody or antigen-binding fragment thereof at a cysteine residue. In some embodiments, the cathepsin-L cleavable linker is attached to the antibody or antigen-binding fragment thereof at a glutamine residue.

In some embodiments of an ADC with a cathepsin-L cleavable linker and an antibody or antigen-binding fragment or variant thereof that comprises VL CDRs of SEQ ID NOs: 29, 10, and 11 and VH CDRs of SEQ ID NOs: 15, 26, and 5, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cathepsin-L cleavable linker, and A is an antibody or antigen-binding fragment or variant thereof that comprises heavy chain variable region (VH) with an amino acid sequence of SEQ ID NO:69 and a light chain variable region (VL) with an amino acid sequence of SEQ ID NO:90. In some embodiments, the cathepsin-L cleavable linker comprises a -Phe-Gln- dipeptide. In some embodiments, the cathepsin-L cleavable linker comprises a -Val-Gln- dipeptide. In some embodiments, the cathepsin-L cleavable linker comprises a -Leu-Gln- dipeptide. In some embodiments, the cathepsin-L cleavable linker comprises a -Tyr-Met- dipeptide. In some embodiments, the cathepsin-L cleavable linker comprises a -Phe-Arg- dipeptide. In some embodiments, the cathepsin-L cleavable linker comprises a -Phe-Gly- dipeptide. In some embodiments, the cathepsin-L comprises cleavable linker comprises a -Trp-Thr- dipeptide. In some embodiments, the cathepsin-L cleavable linker comprises a -Tyr-Gly- dipeptide. In some embodiments, the cathepsin-L cleavable linker comprises a -Phe-Thr- dipeptide. In some embodiments, the cathepsin-L cleavable linker comprises a -Val-Gly- dipeptide. In some embodiments, the cathepsin-L cleavable linker is attached to the antibody or antigen-binding fragment thereof at a lysine residue. In some embodiments, the cathepsin-L cleavable linker is attached to the antibody or antigen-binding fragment thereof at a cysteine residue. In some embodiments, the cathepsin-L cleavable linker is attached to the antibody or antigen-binding fragment thereof at a glutamine residue.

In some embodiments of an ADC with a cathepsin-L cleavable linker and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cathepsin-L cleavable linker, and A is an antibody or antigen-binding fragment or variant thereof that comprises heavy chain variable region (VH) with an amino acid sequence of SEQ ID NO:127 and a light chain variable region (VL) with an amino acid sequence of SEQ ID NO:126. In some embodiments, the cathepsin-L cleavable linker comprises a -Phe-Gln- dipeptide. In some embodiments, the cathepsin-L cleavable linker comprises a -Val-Gln- dipeptide. In some embodiments, the cathepsin-L cleavable linker comprises a -Leu-Gln- dipeptide. In some embodiments, the cathepsin-L cleavable linker comprises a -Tyr-Met- dipeptide. In some embodiments, the cathepsin-L cleavable linker comprises a -Phe-Arg- dipeptide. In some embodiments, the cathepsin-L cleavable linker comprises a -Phe-Gly- dipeptide. In some embodiments, the cathepsin-L comprises cleavable linker comprises a -Trp-Thr- dipeptide. In some embodiments, the cathepsin-L cleavable linker comprises a -Tyr-Gly- dipeptide. In some embodiments, the cathepsin-L cleavable linker comprises a -Phe-Thr- dipeptide. In some embodiments, the cathepsin-L cleavable linker comprises a -Val-Gly- dipeptide. In some embodiments, the cathepsin-L cleavable linker is attached to the antibody or antigen-binding fragment thereof at a lysine residue. In some embodiments, the cathepsin-L cleavable linker is attached to the antibody or antigen-binding fragment thereof at a cysteine residue. In some embodiments, the cathepsin-L cleavable linker is attached to the antibody or antigen-binding fragment thereof at a glutamine residue.

In some embodiments of an ADC with a cathepsin-L cleavable linker and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker and an antibody or antigen-binding fragment or variant thereof that comprises Heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Phe-Gln- dipeptide, and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Phe-Gln-dipeptide is attached to the antibody or antigen-binding fragment thereof at a lysine residue. In some embodiments, the cleavable linker comprising a -Phe-Gln- dipeptide is attached to the antibody or antigen-binding fragment thereof at a cysteine residue. In some embodiments, the cleavable linker comprising a -Phe-Gln- dipeptide is attached to the antibody or antigen-binding fragment thereof at a glutamine residue.

In some embodiments of an ADC with a cleavable linker comprising a -Phe-Gln-dipeptide and an antibody or antigen-binding fragment or variant thereof that comprises VL CDRs of SEQ ID NOs: 61, 62, and 63 and VH CDRs of SEQ ID NOs: 58, 59, and 60, the cleavable linker comprising a -Phe-Gln- dipeptide is an un-branched linker. In some embodiments, the cleavable linker comprising a -Phe-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Phe-Gln- dipeptide and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Phe-Gln- dipeptide and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Phe-Gln- dipeptide, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Phe-Gln-dipeptide is attached to the antibody or antigen-binding fragment thereof at a lysine residue. In some embodiments, the cleavable linker comprising a -Phe-Gln- dipeptide is attached to the antibody or antigen-binding fragment thereof at a cysteine residue. In some embodiments, the cleavable linker comprising a -Phe-Gln- dipeptide is attached to the antibody or antigen-binding fragment thereof at a glutamine residue.

In some embodiments of an ADC with a cleavable linker comprising a -Phe-Gln-dipeptide and an antibody or antigen-binding fragment or variant thereof that comprises VL CDRs of SEQ ID NOs: 9, 10, and 11 and VH CDRs of SEQ ID NOs: 15, 4, and 5, the cleavable linker comprising a -Phe-Gln- dipeptide is an un-branched linker. In some embodiments, the cleavable linker comprising a -Phe-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Phe-Gln- dipeptide and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Phe-Gln- dipeptide and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Phe-Gln- dipeptide, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the cleavable linker comprising a -Phe-Gln-dipeptide is attached to the antibody or antigen-binding fragment thereof at a lysine residue. In some embodiments, the cleavable linker comprising a -Phe-Gln- dipeptide is attached to the antibody or antigen-binding fragment thereof at a cysteine residue. In some embodiments, the cleavable linker comprising a -Phe-Gln- dipeptide is attached to the antibody or antigen-binding fragment thereof at a glutamine residue.

In some embodiments of an ADC with a cleavable linker comprising a -Phe-Gln-dipeptide and an antibody or antigen-binding fragment or variant thereof that comprises VL CDRs of SEQ ID NOs: 29, 10, and 11 and VH CDRs of SEQ ID NOs: 15, 26, and 5, the cleavable linker comprising a -Phe-Gln- dipeptide is an un-branched linker. In some embodiments, the cleavable linker comprising a -Phe-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Phe-Gln- dipeptide and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Phe-Gln- dipeptide and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Phe-Gln- dipeptide, and A is an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90. In some embodiments, the cleavable linker comprising a -Phe-Gln- dipeptide is attached to the antibody or antigen-binding fragment thereof at a lysine residue. In some embodiments, the cleavable linker comprising a -Phe-Gln-dipeptide is attached to the antibody or antigen-binding fragment thereof at a cysteine residue. In some embodiments, the cleavable linker comprising a -Phe-Gln- dipeptide is attached to the antibody or antigen-binding fragment thereof at a glutamine residue.

In some embodiments of an ADC with a cleavable linker comprising a -Phe-Gln-dipeptide and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90, the cleavable linker comprising a -Phe-Gln- dipeptide is an un-branched linker. In some embodiments, the cleavable linker comprising a -Phe-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Phe-Gln- dipeptide and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Phe-Gln- dipeptide and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Phe-Gln- dipeptide, and A is an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126. In some embodiments, the cleavable linker comprising a -Phe-Gln- dipeptide is attached to the antibody or antigen-binding fragment thereof at a lysine residue. In some embodiments, the cleavable linker comprising a -Phe-Gln-dipeptide is attached to the antibody or antigen-binding fragment thereof at a cysteine residue. In some embodiments, the cleavable linker comprising a -Phe-Gln- dipeptide is attached to the antibody or antigen-binding fragment thereof at a glutamine residue.

In some embodiments of an ADC with a cleavable linker comprising a -Phe-Gln-dipeptide and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126, the cleavable linker comprising a -Phe-Gln- dipeptide is an un-branched linker. In some embodiments, the cleavable linker comprising a -Phe-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Phe-Gln- dipeptide and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Phe-Gln- dipeptide and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Val-Gln- dipeptide, and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Val-Gln-dipeptide is attached to the antibody or antigen-binding fragment thereof at a lysine residue. In some embodiments, the cleavable linker comprising a -Val-Gln- dipeptide is attached to the antibody or antigen-binding fragment thereof at a cysteine residue. In some embodiments, the cleavable linker comprising a -Val-Gln- dipeptide is attached to the antibody or antigen-binding fragment thereof at a glutamine residue.

In some embodiments of an ADC with a cleavable linker comprising a -Val-Gln-dipeptide and an antibody or antigen-binding fragment or variant thereof that comprises VL CDRs of SEQ ID NOs: 61, 62, and 63 and VH CDRs of SEQ ID NOs: 58, 59, and 60, the cleavable linker comprising a -Val-Gln- dipeptide is an un-branched linker. In some embodiments, the cleavable linker comprising a -Val-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Val-Gln- dipeptide and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Val-Gln- dipeptide and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Val-Gln- dipeptide, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Val-Gln-dipeptide is attached to the antibody or antigen-binding fragment thereof at a lysine residue. In some embodiments, the cleavable linker comprising a -Val-Gln- dipeptide is attached to the antibody or antigen-binding fragment thereof at a cysteine residue. In some embodiments, the cleavable linker comprising a -Val-Gln- dipeptide is attached to the antibody or antigen-binding fragment thereof at a glutamine residue.

In some embodiments of an ADC with a cleavable linker comprising a -Val-Gln-dipeptide and an antibody or antigen-binding fragment or variant thereof that comprises VL CDRs of SEQ ID NOs: 9, 10, and 11 and VH CDRs of SEQ ID NOs: 15, 4, and 5, the cleavable linker comprising a -Val-Gln- dipeptide is an un-branched linker. In some embodiments, the cleavable linker comprising a -Val-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Val-Gln- dipeptide and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Val-Gln- dipeptide and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Val-Gln- dipeptide, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the cleavable linker comprising a -Val-Gln-dipeptide is attached to the antibody or antigen-binding fragment thereof at a lysine residue. In some embodiments, the cleavable linker comprising a -Val-Gln- dipeptide is attached to the antibody or antigen-binding fragment thereof at a cysteine residue. In some embodiments, the cleavable linker comprising a -Val-Gln- dipeptide is attached to the antibody or antigen-binding fragment thereof at a glutamine residue.

In some embodiments of an ADC with a cleavable linker comprising a -Val-Gln-dipeptide and an antibody or antigen-binding fragment or variant thereof that comprises VL CDRs of SEQ ID NOs: 29, 10, and 11 and VH CDRs of SEQ ID NOs: 15, 26, and 5, the cleavable linker comprising a -Val-Gln- dipeptide is an un-branched linker. In some embodiments, the cleavable linker comprising a -Val-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Val-Gln- dipeptide and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Val-Gln- dipeptide and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Val-Gln- dipeptide, and A is an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90. In some embodiments, the cleavable linker comprising a -Val-Gln- dipeptide is attached to the antibody or antigen-binding fragment thereof at a lysine residue. In some embodiments, the cleavable linker comprising a -Val-Gln-dipeptide is attached to the antibody or antigen-binding fragment thereof at a cysteine residue. In some embodiments, the cleavable linker comprising a -Val-Gln- dipeptide is attached to the antibody or antigen-binding fragment thereof at a glutamine residue.

In some embodiments of an ADC with a cleavable linker comprising a -Val-Gln-dipeptide and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90, the cleavable linker comprising a -Val-Gln- dipeptide is an un-branched linker. In some embodiments, the cleavable linker comprising a -Val-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Val-Gln- dipeptide and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Val-Gln- dipeptide and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Val-Gln- dipeptide, and A is an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126. In some embodiments, the cleavable linker comprising a -Val-Gln- dipeptide is attached to the antibody or antigen-binding fragment thereof at a lysine residue. In some embodiments, the cleavable linker comprising a -Val-Gln-dipeptide is attached to the antibody or antigen-binding fragment thereof at a cysteine residue. In some embodiments, the cleavable linker comprising a -Val-Gln- dipeptide is attached to the antibody or antigen-binding fragment thereof at a glutamine residue.

In some embodiments of an ADC with a cleavable linker comprising a -Val-Gln-dipeptide and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126, the cleavable linker comprising a -Val-Gln- dipeptide is an un-branched linker. In some embodiments, the cleavable linker comprising a -Val-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Val-Gln- dipeptide and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Val-Gln- dipeptide and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Leu-Gln- dipeptide, and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Leu-Gln-dipeptide is attached to the antibody or antigen-binding fragment thereof at a lysine residue. In some embodiments, the cleavable linker comprising a -Leu-Gln- dipeptide is attached to the antibody or antigen-binding fragment thereof at a cysteine residue. In some embodiments, the cleavable linker comprising a -Leu-Gln- dipeptide is attached to the antibody or antigen-binding fragment thereof at a glutamine residue.

In some embodiments of an ADC with a cleavable linker comprising a -Leu-Gln-dipeptide and an antibody or antigen-binding fragment or variant thereof that comprises VL CDRs of SEQ ID NOs: 61, 62, and 63 and VH CDRs of SEQ ID NOs: 58, 59, and 60, the cleavable linker comprising a -Leu-Gln- dipeptide is an un-branched linker. In some embodiments, the cleavable linker comprising a -Leu-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Leu-Gln- dipeptide and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Leu-Gln- dipeptide and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Leu-Gln- dipeptide, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Leu-Gln-dipeptide is attached to the antibody or antigen-binding fragment thereof at a lysine residue. In some embodiments, the cleavable linker comprising a -Leu-Gln- dipeptide is attached to the antibody or antigen-binding fragment thereof at a cysteine residue. In some embodiments, the cleavable linker comprising a -Leu-Gln- dipeptide is attached to the antibody or antigen-binding fragment thereof at a glutamine residue.

In some embodiments of an ADC with a cleavable linker comprising a -Leu-Gln-dipeptide and an antibody or antigen-binding fragment or variant thereof that comprises VL CDRs of SEQ ID NOs: 9, 10, and 11 and VH CDRs of SEQ ID NOs: 15, 4, and 5, the cleavable linker comprising a -Leu-Gln- dipeptide is an un-branched linker. In some embodiments, the cleavable linker comprising a -Leu-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Leu-Gln- dipeptide and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Leu-Gln- dipeptide and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Leu-Gln- dipeptide, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the cleavable linker comprising a -Leu-Gln-dipeptide is attached to the antibody or antigen-binding fragment thereof at a lysine residue. In some embodiments, the cleavable linker comprising a -Leu-Gln- dipeptide is attached to the antibody or antigen-binding fragment thereof at a cysteine residue. In some embodiments, the cleavable linker comprising a -Leu-Gln- dipeptide is attached to the antibody or antigen-binding fragment thereof at a glutamine residue.

In some embodiments of an ADC with a cleavable linker comprising a -Leu-Gln-dipeptide and an antibody or antigen-binding fragment or variant thereof that comprises VL CDRs of SEQ ID NOs: 29, 10, and 11 and VH CDRs of SEQ ID NOs: 15, 26, and 5, the cleavable linker comprising a -Leu-Gln- dipeptide is an un-branched linker. In some embodiments, the cleavable linker comprising a -Leu-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Leu-Gln- dipeptide and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Leu-Gln- dipeptide and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Leu-Gln- dipeptide, and A is an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90. In some embodiments, the cleavable linker comprising a -Leu-Gln- dipeptide is attached to the antibody or antigen-binding fragment thereof at a lysine residue. In some embodiments, the cleavable linker comprising a -Leu-Gln-dipeptide is attached to the antibody or antigen-binding fragment thereof at a cysteine residue. In some embodiments, the cleavable linker comprising a -Leu-Gln- dipeptide is attached to the antibody or antigen-binding fragment thereof at a glutamine residue.

In some embodiments of an ADC with a cleavable linker comprising a -Leu-Gln-dipeptide and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90, the cleavable linker comprising a -Leu-Gln- dipeptide is an un-branched linker. In some embodiments, the cleavable linker comprising a -Leu-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Leu-Gln- dipeptide and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Leu-Gln- dipeptide and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a cleavable linker comprising a -Leu-Gln- dipeptide, and A is an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126. In some embodiments, the cleavable linker comprising a -Leu-Gln- dipeptide is attached to the antibody or antigen-binding fragment thereof at a lysine residue. In some embodiments, the cleavable linker comprising a -Leu-Gln-dipeptide is attached to the antibody or antigen-binding fragment thereof at a cysteine residue. In some embodiments, the cleavable linker comprising a -Leu-Gln- dipeptide is attached to the antibody or antigen-binding fragment thereof at a glutamine residue.

In some embodiments of an ADC with a cleavable linker comprising a -Leu-Gln-dipeptide and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126, the cleavable linker comprising a -Leu-Gln- dipeptide is an un-branched linker. In some embodiments, the cleavable linker comprising a -Leu-Gln- dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cleavable linker comprising a -Leu-Gln- dipeptide and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cleavable linker comprising a -Leu-Gln- dipeptide and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a disulfide cleavable linker, and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the disulfide cleavable linker is attached to the antibody or antigen-binding fragment thereof at a lysine residue. In some embodiments, the disulfide cleavable linker is attached to the antibody or antigen-binding fragment thereof at a cysteine residue. In some embodiments, the disulfide cleavable linker is attached to the antibody or antigen-binding fragment thereof at a glutamine residue.

In some embodiments of an ADC with a disulfide cleavable linker and an antibody or antigen-binding fragment or variant thereof that comprises VL CDRs of SEQ ID NOs: 61, 62, and 63 and VH CDRs of SEQ ID NOs: 58, 59, and 60, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a disulfide cleavable linker and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a disulfide cleavable linker, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the disulfide cleavable linker is attached to the antibody or antigen-binding fragment thereof at a lysine residue. In some embodiments, the disulfide cleavable linker is attached to the antibody or antigen-binding fragment thereof at a cysteine residue. In some embodiments, the disulfide cleavable linker is attached to the antibody or antigen-binding fragment thereof at a glutamine residue.

In some embodiments of an ADC with a disulfide cleavable linker and an antibody or antigen-binding fragment or variant thereof that comprises VL CDRs of SEQ ID NOs: 9, 10, and 11 and VH CDRs of SEQ ID NOs: 15, 4, and 5, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a disulfide cleavable linker and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a disulfide cleavable linker, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the disulfide cleavable linker is attached to the antibody or antigen-binding fragment thereof at a lysine residue. In some embodiments, the disulfide cleavable linker is attached to the antibody or antigen-binding fragment thereof at a cysteine residue. In some embodiments, the disulfide cleavable linker is attached to the antibody or antigen-binding fragment thereof at a glutamine residue.

In some embodiments of an ADC with a disulfide cleavable linker and an antibody or antigen-binding fragment or variant thereof that comprises VL CDRs of SEQ ID NOs: 29, 10, and 11 and VH CDRs of SEQ ID NOs: 15, 26, and 5, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a disulfide cleavable linker and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a disulfide cleavable linker, and A is an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90. In some embodiments, the disulfide cleavable linker is attached to the antibody or antigen-binding fragment thereof at a lysine residue. In some embodiments, the disulfide cleavable linker is attached to the antibody or antigen-binding fragment thereof at a cysteine residue. In some embodiments, the disulfide cleavable linker is attached to the antibody or antigen-binding fragment thereof at a glutamine residue.

In some embodiments of an ADC with a disulfide cleavable linker and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a disulfide cleavable linker and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a disulfide cleavable linker, and A is an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126. In some embodiments, the disulfide cleavable linker is attached to the antibody or antigen-binding fragment thereof at a lysine residue. In some embodiments, the disulfide cleavable linker is attached to the antibody or antigen-binding fragment thereof at a cysteine residue. In some embodiments, the disulfide cleavable linker is attached to the antibody or antigen-binding fragment thereof at a glutamine residue.

In some embodiments of an ADC with a disulfide cleavable linker and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a disulfide cleavable linker and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a hindered disulfide cleavable linker, and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the hindered disulfide cleavable linker is attached to the antibody or antigen-binding fragment thereof at a lysine residue. In some embodiments, the hindered disulfide cleavable linker is attached to the antibody or antigen-binding fragment thereof at a cysteine residue. In some embodiments, the hindered disulfide cleavable linker is attached to the antibody or antigen-binding fragment thereof at a glutamine residue.

In some embodiments of an ADC with a hindered disulfide cleavable linker and an antibody or antigen-binding fragment or variant thereof that comprises VL CDRs of SEQ ID NOs: 61, 62, and 63 and VH CDRs of SEQ ID NOs: 58, 59, and 60, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a hindered disulfide cleavable linker and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a hindered disulfide cleavable linker, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the hindered disulfide cleavable linker is attached to the antibody or antigen-binding fragment thereof at a lysine residue. In some embodiments, the hindered disulfide cleavable linker is attached to the antibody or antigen-binding fragment thereof at a cysteine residue. In some embodiments, the hindered disulfide cleavable linker is attached to the antibody or antigen-binding fragment thereof at a glutamine residue.

In some embodiments of an ADC with a hindered disulfide cleavable linker and an antibody or antigen-binding fragment or variant thereof that comprises VL CDRs of SEQ ID NOs: 9, 10, and 11 and VH CDRs of SEQ ID NOs: 15, 4, and 5, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a hindered disulfide cleavable linker and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a hindered disulfide cleavable linker, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the hindered disulfide cleavable linker is attached to the antibody or antigen-binding fragment thereof at a lysine residue. In some embodiments, the hindered disulfide cleavable linker is attached to the antibody or antigen-binding fragment thereof at a cysteine residue. In some embodiments, the hindered disulfide cleavable linker is attached to the antibody or antigen-binding fragment thereof at a glutamine residue.

In some embodiments of an ADC with a hindered disulfide cleavable linker and an antibody or antigen-binding fragment or variant thereof that comprises VL CDRs of SEQ ID NOs: 29, 10, and 11 and VH CDRs of SEQ ID NOs: 15, 26, and 5, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a hindered disulfide cleavable linker and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a hindered disulfide cleavable linker, and A is an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90. In some embodiments, the hindered disulfide cleavable linker is attached to the antibody or antigen-binding fragment thereof at a lysine residue. In some embodiments, the hindered disulfide cleavable linker is attached to the antibody or antigen-binding fragment thereof at a cysteine residue. In some embodiments, the hindered disulfide cleavable linker is attached to the antibody or antigen-binding fragment thereof at a glutamine residue.

In some embodiments of an ADC with a hindered disulfide cleavable linker and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a hindered disulfide cleavable linker and an antibody or antigen-binding fragment or variant thereof that comprises a heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, L is a hindered disulfide cleavable linker, and A is an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126. In some embodiments, the hindered disulfide cleavable linker is attached to the antibody or antigen-binding fragment thereof at a lysine residue. In some embodiments, the hindered disulfide cleavable linker is attached to the antibody or antigen-binding fragment thereof at a cysteine residue. In some embodiments, the hindered disulfide cleavable linker is attached to the antibody or antigen-binding fragment thereof at a glutamine residue.

In some embodiments of an ADC with a hindered disulfide cleavable linker and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a hindered disulfide cleavable linker and an antibody or antigen-binding fragment or variant thereof that comprises a heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. G. Exemplary Conjugation Chemistry-Linkers

In embodiments, the present disclosure provides an antibody-drug conjugate (ADC) having the formula A-(L-Pᵣ)_{q}, wherein: A is a 3E10 antibody or antigen-binding fragment or variant thereof, L is a linker, P is a payload as described herein, r is an integer from 1 to 4, and q is an integer from 1 to 16.

In embodiments, L is a cathepsin-L cleavable linker attached to the 3E10 antibody or antigen-binding fragment thereof at a lysine residue. In some embodiments, the cathepsin-L cleavable linker comprises a -Phe-Gln- dipeptide. In embodiments, the cathepsin-L cleavable linker comprises a -Val-Gln- dipeptide. In embodiments, the cathepsin-L cleavable linker comprises a -Leu-Gln- dipeptide. In embodiments, the cathepsin-L comprises cleavable linker comprises a -Tyr-Met- dipeptide. In embodiments, the cathepsin-L comprises cleavable linker comprises a -Phe-Arg- dipeptide. In embodiments, the cathepsin-L comprises cleavable linker comprises a -Phe-Gly- dipeptide. In embodiments, the cathepsin-L comprises cleavable linker comprises a -Trp-Thr- dipeptide. In embodiments, the cathepsin-L comprises cleavable linker comprises a -Tyr-Gly- dipeptide. In embodiments, the cathepsin-L comprises cleavable linker comprises a -Phe-Thr- dipeptide. In embodiments, the cathepsin-L comprises cleavable linker comprises a -Val-Gly- dipeptide. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker attached to the 3E10 antibody or antigen-binding fragment thereof at a lysine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a cathepsin-L cleavable linker attached to the 3E10 antibody or antigen-binding fragment thereof at a cysteine residue. In some embodiments, the cathepsin-L cleavable linker comprises a -Phe-Gln- dipeptide. In embodiments, the cathepsin-L cleavable linker comprises a -Val-Gln- dipeptide. In embodiments, the cathepsin-L cleavable linker comprises a -Leu-Gln- dipeptide. In embodiments, the cathepsin-L comprises cleavable linker comprises a -Tyr-Met- dipeptide. In embodiments, the cathepsin-L comprises cleavable linker comprises a -Phe-Arg- dipeptide. In embodiments, the cathepsin-L comprises cleavable linker comprises a -Phe-Gly- dipeptide. In embodiments, the cathepsin-L comprises cleavable linker comprises a -Trp-Thr- dipeptide. In embodiments, the cathepsin-L comprises cleavable linker comprises a -Tyr-Gly- dipeptide. In embodiments, the cathepsin-L comprises cleavable linker comprises a -Phe-Thr- dipeptide. In embodiments, the cathepsin-L comprises cleavable linker comprises a -Val-Gly- dipeptide. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker attached to the 3E10 antibody or antigen-binding fragment thereof at a cysteine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, the present disclosure provides pharmaceutical compositions comprising an antibody-drug conjugate (ADC) having the formula A-(L-Pᵣ)_{q}, where: A is a 3E10 antibody or antigen-binding fragment thereof as described herein (e.g., as described in the section titled 3E10 Antibodies and Antigen-Binding Fragments), L is a linker, P is a payload moiety as described herein, r is an integer from 1 to 4 and q is an integer from 1 to 16.

In embodiments, L is a cathepsin-L cleavable linker attached to the 3E10 antibody or antigen-binding fragment thereof at a glutamine residue. In some embodiments, the cathepsin-L cleavable linker comprises a -Phe-Gln- dipeptide. In embodiments, the cathepsin-L cleavable linker comprises a -Val-Gln- dipeptide. In embodiments, the cathepsin-L cleavable linker comprises a -Leu-Gln- dipeptide. In embodiments, the cathepsin-L comprises cleavable linker comprises a -Tyr-Met- dipeptide. In embodiments, the cathepsin-L comprises cleavable linker comprises a -Phe-Arg- dipeptide. In embodiments, the cathepsin-L comprises cleavable linker comprises a -Phe-Gly- dipeptide. In embodiments, the cathepsin-L comprises cleavable linker comprises a -Trp-Thr- dipeptide. In embodiments, the cathepsin-L comprises cleavable linker comprises a -Tyr-Gly- dipeptide. In embodiments, the cathepsin-L comprises cleavable linker comprises a -Phe-Thr- dipeptide. In embodiments, the cathepsin-L comprises cleavable linker comprises a -Val-Gly- dipeptide. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker attached to the 3E10 antibody or antigen-binding fragment thereof at a glutamine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a cathepsin-L cleavable linker comprising a -Phe-Gln- dipeptide attached to the 3E10 antibody or antigen-binding fragment thereof at a lysine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker comprising a -Phe-Gln-dipeptide attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker comprising a -Phe-Gln- dipeptide attached to the 3E10 antibody or antigen-binding fragment thereof at a lysine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker comprising a -Phe-Gln- dipeptide attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a cathepsin-L cleavable linker comprising a -Phe-Gln- dipeptide attached to the 3E10 antibody or antigen-binding fragment thereof at a cysteine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker comprising a -Phe-Gln-dipeptide attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker comprising a -Phe-Gln- dipeptide attached to the 3E10 antibody or antigen-binding fragment thereof at a cysteine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker comprising a -Phe-Gln- dipeptide attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a cathepsin-L cleavable linker comprising a -Phe-Gln- dipeptide attached to the 3E10 antibody or antigen-binding fragment thereof at a glutamine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker comprising a -Phe-Gln-dipeptide attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker comprising a -Phe-Gln- dipeptide attached to the 3E10 antibody or antigen-binding fragment thereof at a glutamine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker comprising a -Phe-Gln- dipeptide attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a cathepsin-L cleavable linker comprising a -Val-Gln- dipeptide attached to the 3E10 antibody or antigen-binding fragment thereof at a lysine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker comprising a -Val-Gln-dipeptide attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker comprising a -Val-Gln- dipeptide attached to the 3E10 antibody or antigen-binding fragment thereof at a lysine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker comprising a -Val-Gln- dipeptide attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract 28s cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a cathepsin-L cleavable linker comprising a -Val-Gln- dipeptide attached to the 3E10 antibody or antigen-binding fragment thereof at a cysteine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker comprising a -Val-Gln-dipeptide attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker comprising a -Val-Gln- dipeptide attached to the 3E10 antibody or antigen-binding fragment thereof at a cysteine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker comprising a -Val-Gln- dipeptide attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a cathepsin-L cleavable linker comprising a -Val-Gln- dipeptide attached to the 3E10 antibody or antigen-binding fragment thereof at a glutamine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker comprising a -Val-Gln-dipeptide attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker comprising a -Val-Gln- dipeptide attached to the 3E10 antibody or antigen-binding fragment thereof at a glutamine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker comprising a -Val-Gln- dipeptide attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a cathepsin-L cleavable linker comprising a -Leu-Gln- dipeptide attached to the 3E10 antibody or antigen-binding fragment thereof at a lysine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker comprising a -Leu-Gln-dipeptide attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker comprising a -Leu-Gln- dipeptide attached to the 3E10 antibody or antigen-binding fragment thereof at a lysine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker comprising a -Leu-Gln- dipeptide attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a cathepsin-L cleavable linker comprising a -Leu-Gln- dipeptide attached to the 3E10 antibody or antigen-binding fragment thereof at a cysteine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker comprising a -Leu-Gln-dipeptide attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker comprising a -Leu-Gln- dipeptide attached to the 3E10 antibody or antigen-binding fragment thereof at a cysteine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker comprising a -Leu-Gln- dipeptide attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a cathepsin-L cleavable linker comprising a -Leu-Gln- dipeptide attached to the 3E10 antibody or antigen-binding fragment thereof at a glutamine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a cathepsin-L cleavable linker comprising a -Leu-Gln-dipeptide attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a cathepsin-L cleavable linker comprising a -Leu-Gln- dipeptide attached to the 3E10 antibody or antigen-binding fragment thereof at a glutamine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a cathepsin-L cleavable linker comprising a -Leu-Gln- dipeptide attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a 2% biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a disulfide cleavable linker attached to the 3E10 antibody or antigen-binding fragment thereof at a lysine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a disulfide cleavable linker attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker attached to the 3E10 antibody or antigen-binding fragment thereof at a lysine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a disulfide cleavable linker attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a disulfide cleavable linker attached to the 3E10 antibody or antigen-binding fragment thereof at a cysteine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a disulfide cleavable linker attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker attached to the 3E10 antibody or antigen-binding fragment thereof at a cysteine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a disulfide cleavable linker attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a disulfide cleavable linker attached to the 3E10 antibody or antigen-binding fragment thereof at a glutamine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a disulfide cleavable linker attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a disulfide cleavable linker attached to the 3E10 antibody or antigen-binding fragment thereof at a glutamine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a disulfide cleavable linker attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a hindered disulfide cleavable linker attached to the 3E10 antibody or antigen-binding fragment thereof at a lysine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a hindered disulfide cleavable linker attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker attached to the 3E10 antibody or antigen-binding fragment thereof at a lysine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a hindered disulfide cleavable linker attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a hindered disulfide cleavable linker attached to the 3E10 antibody or antigen-binding fragment thereof at a cysteine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a hindered disulfide cleavable linker attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker attached to the 3E10 antibody or antigen-binding fragment thereof at a cysteine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a hindered disulfide cleavable linker attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In embodiments, L is a hindered disulfide cleavable linker attached to the 3E10 antibody or antigen-binding fragment thereof at a glutamine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:127 and a light chain variable region (VL) with an amino acid sequence of SEQ ID NO:126.

In some embodiments of an ADC with a hindered disulfide cleavable linker attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branced linker has four arms. In some embodiments, the branched linker has at least 2 arms, at least 3 arms, at least 4 arms, or more. In some embodiments, the branced linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a hindered disulfide cleavable linker attached to the 3E10 antibody or antigen-binding fragment thereof at a glutamine residue are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a hindered disulfide cleavable linker attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

### H. Exemplary ADCs

In some embodiments, the present disclosure provides pharmaceutical compositions comprising an antibody-drug conjugate (ADC) having the formula A-(L-Pᵣ)_{q}, where: A is a 3E10 antibody or antigen-binding fragment thereof (e.g., as described in the section titled 3E10 Antibodies and Antigen-Binding Fragments), L is a linker as described herein, P is a payload moiety, r is an integer from 1 to 4 and q is an integer from 1 to 16. In some embodiments, P is a topoisomerase inhibitor, and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the topoisomerase inhibitor is attached to the antibody or antigen-binding fragment thereof at a lysine residue. In some embodiments, the topoisomerase inhibitor is attached to the antibody or antigen-binding fragment thereof at a cysteine residue. In some embodiments, the topoisomerase inhibitor is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with a topoisomerase inhibitor and an antibody or antigen-binding fragment or variant thereof that comprises VL CDRs of SEQ ID NOs: 61, 62, and 63 and VH CDRs of SEQ ID NOs: 58, 59, and 60, the linker connecting the antibody or antigen binding fragment thereof to the topoisomerase inhibitor is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a topoisomerase inhibitor and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a topoisomerase inhibitor and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is a topoisomerase inhibitor, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the topoisomerase inhibitor is attached to the antibody or antigen-binding fragment thereof at a lysine residue. In some embodiments, the topoisomerase inhibitor is attached to the antibody or antigen-binding fragment thereof at a cysteine residue. In some embodiments, the topoisomerase inhibitor is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with a topoisomerase inhibitor and an antibody or antigen-binding fragment or variant thereof that comprises VL CDRs of SEQ ID NOs: 9, 10, and 11 and VH CDRs of SEQ ID NOs: 15, 4, and 5, the linker connecting the antibody or antigen binding fragment thereof to the topoisomerase inhibitor is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a topoisomerase inhibitor and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a topoisomerase inhibitor and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is a topoisomerase inhibitor, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the topoisomerase inhibitor is attached to the antibody or antigen-binding fragment thereof at a lysine residue. In some embodiments, the topoisomerase inhibitor is attached to the antibody or antigen-binding fragment thereof at a cysteine residue. In some embodiments, the topoisomerase inhibitor is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with a topoisomerase inhibitor and an antibody or antigen-binding fragment or variant thereof that comprises VL CDRs of SEQ ID NOs: 29, 10, and 11 and VH CDRs of SEQ ID NOs: 15, 26, and 5, the linker connecting the antibody or antigen binding fragment thereof to the topoisomerase inhibitor is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a topoisomerase inhibitor and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a topoisomerase inhibitor and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is a topoisomerase inhibitor, and A is an antibody or antigen-binding fragment or variant thereof that comprises heavy chain variable region (VH) with an amino acid sequence of SEQ ID NO:69 and a light chain variable region (VL) with an amino acid sequence of SEQ ID NO:90. In some embodiments, the topoisomerase inhibitor is attached to the antibody or antigen-binding fragment thereof at a lysine residue. In some embodiments, the topoisomerase inhibitor is attached to the antibody or antigen-binding fragment thereof at a cysteine residue. In some embodiments, the topoisomerase inhibitor is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with a topoisomerase inhibitor and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, the linker connecting the antibody or antigen binding fragment thereof to the topoisomerase inhibitor is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a topoisomerase inhibitor and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a topoisomerase inhibitor and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is a topoisomerase inhibitor, and A is an antibody or antigen-binding fragment or variant thereof that comprises heavy chain variable region (VH) with an amino acid sequence of SEQ ID NO:127 and a light chain variable region (VL) with an amino acid sequence of SEQ ID NO:126. In some embodiments, the topoisomerase inhibitor is attached to the antibody or antigen-binding fragment thereof at a lysine residue. In some embodiments, the topoisomerase inhibitor is attached to the antibody or antigen-binding fragment thereof at a cysteine residue. In some embodiments, the topoisomerase inhibitor is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with a topoisomerase inhibitor and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126, the linker connecting the antibody or antigen binding fragment thereof to the topoisomerase inhibitor is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a topoisomerase inhibitor and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a topoisomerase inhibitor and an antibody or antigen-binding fragment or variant thereof that comprises Heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is PNU-159682, and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the PNU-159682 is attached to the antibody or antigen-binding fragment thereof at a lysine residue. In some embodiments, the PNU-159682 is attached to the antibody or antigen-binding fragment thereof at a cysteine residue. In some embodiments, the PNU-159682 is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with PNU-159682 and an antibody or antigen-binding fragment or variant thereof that comprises VL CDRs of SEQ ID NOs: 61, 62, and 63 and VH CDRs of SEQ ID NOs: 58, 59, and 60, the linker connecting the antibody or antigen binding fragment thereof to the PNU-159682 is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with PNU-159682 and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with PNU-159682 and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is PNU-159682, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the PNU-159682 is attached to the antibody or antigen-binding fragment thereof at a lysine residue. In some embodiments, the PNU-159682 is attached to the antibody or antigen-binding fragment thereof at a cysteine residue. In some embodiments, the PNU-159682 is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with PNU-159682 and an antibody or antigen-binding fragment or variant thereof that comprises VL CDRs of SEQ ID NOs: 9, 10, and 11 and VH CDRs of SEQ ID NOs: 15, 4, and 5, the linker connecting the antibody or antigen binding fragment thereof to the PNU-159682 is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with PNU-159682 and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with PNU-159682 and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is PNU-159682, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the PNU-159682 is attached to the antibody or antigen-binding fragment thereof at a lysine residue. In some embodiments, the PNU-159682 is attached to the antibody or antigen-binding fragment thereof at a cysteine residue. In some embodiments, the PNU-159682 is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with PNU-159682 and an antibody or antigen-binding fragment or variant thereof that comprises VL CDRs of SEQ ID NOs: 29, 10, and 11 and VH CDRs of SEQ ID NOs: 15, 26, and 5, the linker connecting the antibody or antigen binding fragment thereof to the PNU-159682 is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with PNU-159682 and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with PNU-159682 and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is PNU-159682, and A is an antibody or antigen-binding fragment or variant thereof that comprises heavy chain variable region (VH) with an amino acid sequence of SEQ ID NO:69 and a light chain variable region (VL) with an amino acid sequence of SEQ ID NO:90. In some embodiments, the PNU-159682 is attached to the antibody or antigen-binding fragment thereof at a lysine residue. In some embodiments, the PNU-159682 is attached to the antibody or antigen-binding fragment thereof at a cysteine residue. In some embodiments, the PNU-159682 is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with PNU-159682 and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, the linker connecting the antibody or antigen binding fragment thereof to the PNU-159682 is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with PNU-159682 and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with PNU-159682 and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is PNU-159682, and A is an antibody or antigen-binding fragment or variant thereof that comprises heavy chain variable region (VH) with an amino acid sequence of SEQ ID NO:127 and a light chain variable region (VL) with an amino acid sequence of SEQ ID NO:126. In some embodiments, the PNU-159682 is attached to the antibody or antigen-binding fragment thereof at a lysine residue. In some embodiments, the PNU-159682 is attached to the antibody or antigen-binding fragment thereof at a cysteine residue. In some embodiments, the PNU-159682 is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with PNU-159682 and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126, the linker connecting the antibody or antigen binding fragment thereof to the PNU-159682 is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with PNU-159682 and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with PNU-159682 and an antibody or antigen-binding fragment or variant thereof that comprises Heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is SN-38, and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the SN-38 is attached to the antibody or antigen-binding fragment thereof at a lysine residue. In some embodiments, the SN-38 is attached to the antibody or antigen-binding fragment thereof at a cysteine residue. In some embodiments, the SN-38 is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with SN-38 and an antibody or antigen-binding fragment or variant thereof that comprises VL CDRs of SEQ ID NOs: 61, 62, and 63 and VH CDRs of SEQ ID NOs: 58, 59, and 60, the linker connecting the antibody or antigen binding fragment thereof to the SN-38 is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with SN-38 and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with SN-38 and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is SN-38, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the SN-38 is attached to the antibody or antigen-binding fragment thereof at a lysine residue. In some embodiments, the SN-38 is attached to the antibody or antigen-binding fragment thereof at a cysteine residue. In some embodiments, the SN-38 is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with SN-38 and an antibody or antigen-binding fragment or variant thereof that comprises VL CDRs of SEQ ID NOs: 9, 10, and 11 and VH CDRs of SEQ ID NOs: 15, 4, and 5, the linker connecting the antibody or antigen binding fragment thereof to the SN-38 is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with SN-38 and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with SN-38 and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is SN-38, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the SN-38 is attached to the antibody or antigen-binding fragment thereof at a lysine residue. In some embodiments, the SN-38 is attached to the antibody or antigen-binding fragment thereof at a cysteine residue. In some embodiments, the SN-38 is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with SN-38 and an antibody or antigen-binding fragment or variant thereof that comprises VL CDRs of SEQ ID NOs: 29, 10, and 11 and VH CDRs of SEQ ID NOs: 15, 26, and 5, the linker connecting the antibody or antigen binding fragment thereof to the SN-38 is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with SN-38 and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with SN-38 and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is SN-38, and A is an antibody or antigen-binding fragment or variant thereof that comprises heavy chain variable region (VH) with an amino acid sequence of SEQ ID NO:69 and a light chain variable region (VL) with an amino acid sequence of SEQ ID NO:90. In some embodiments, the SN-38 is attached to the antibody or antigen-binding fragment thereof at a lysine residue. In some embodiments, the SN-38 is attached to the antibody or antigen-binding fragment thereof at a cysteine residue. In some embodiments, the SN-38 is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with SN-38 and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, the linker connecting the antibody or antigen binding fragment thereof to the SN-38 is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with SN-38 and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with SN-38 and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is SN-38, and A is an antibody or antigen-binding fragment or variant thereof that comprises heavy chain variable region (VH) with an amino acid sequence of SEQ ID NO:127 and a light chain variable region (VL) with an amino acid sequence of SEQ ID NO:126. In some embodiments, the SN-38 is attached to the antibody or antigen-binding fragment thereof at a lysine residue. In some embodiments, the SN-38 is attached to the antibody or antigen-binding fragment thereof at a cysteine residue. In some embodiments, the SN-38 is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with SN-38 and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126, the linker connecting the antibody or antigen binding fragment thereof to the SN-38 is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with SN-38 and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with SN-38 and an antibody or antigen-binding fragment or variant thereof that comprises Heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is a tubulin inhibitor, and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the tubulin inhibitor is attached to the antibody or antigen-binding fragment thereof at a lysine residue. In some embodiments, the tubulin inhibitor is attached to the antibody or antigen-binding fragment thereof at a cysteine residue. In some embodiments, the tubulin inhibitor is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with a tubulin inhibitor and an antibody or antigen-binding fragment or variant thereof that comprises VL CDRs of SEQ ID NOs: 61, 62, and 63 and VH CDRs of SEQ ID NOs: 58, 59, and 60, the linker connecting the antibody or antigen binding fragment thereof to the tubulin inhibitor is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a tubulin inhibitor and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a tubulin inhibitor and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is a tubulin inhibitor, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the tubulin inhibitor is attached to the antibody or antigen-binding fragment thereof at a lysine residue. In some embodiments, the tubulin inhibitor is attached to the antibody or antigen-binding fragment thereof at a cysteine residue. In some embodiments, the tubulin inhibitor is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with a tubulin inhibitor and an antibody or antigen-binding fragment or variant thereof that comprises VL CDRs of SEQ ID NOs: 9, 10, and 11 and VH CDRs of SEQ ID NOs: 15, 4, and 5, the linker connecting the antibody or antigen binding fragment thereof to the tubulin inhibitor is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a tubulin inhibitor and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a tubulin inhibitor and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is a tubulin inhibitor, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the tubulin inhibitor is attached to the antibody or antigen-binding fragment thereof at a lysine residue. In some embodiments, the tubulin inhibitor is attached to the antibody or antigen-binding fragment thereof at a cysteine residue. In some embodiments, the tubulin inhibitor is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with a tubulin inhibitor and an antibody or antigen-binding fragment or variant thereof that comprises VL CDRs of SEQ ID NOs: 29, 10, and 11 and VH CDRs of SEQ ID NOs: 15, 26, and 5, the linker connecting the antibody or antigen binding fragment thereof to the tubulin inhibitor is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a tubulin inhibitor and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a tubulin inhibitor and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is a tubulin inhibitor, and A is an antibody or antigen-binding fragment or variant thereof that comprises heavy chain variable region (VH) with an amino acid sequence of SEQ ID NO:69 and a light chain variable region (VL) with an amino acid sequence of SEQ ID NO:90. In some embodiments, the tubulin inhibitor is attached to the antibody or antigen-binding fragment thereof at a lysine residue. In some embodiments, the tubulin inhibitor is attached to the antibody or antigen-binding fragment thereof at a cysteine residue. In some embodiments, the tubulin inhibitor is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with a tubulin inhibitor and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, the linker connecting the antibody or antigen binding fragment thereof to the tubulin inhibitor is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a tubulin inhibitor and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a tubulin inhibitor and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is a tubulin inhibitor, and A is an antibody or antigen-binding fragment or variant thereof that comprises heavy chain variable region (VH) with an amino acid sequence of SEQ ID NO:127 and a light chain variable region (VL) with an amino acid sequence of SEQ ID NO:126. In some embodiments, the tubulin inhibitor is attached to the antibody or antigen-binding fragment thereof at a lysine residue. In some embodiments, the tubulin inhibitor is attached to the antibody or antigen-binding fragment thereof at a cysteine residue. In some embodiments, the tubulin inhibitor is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with a tubulin inhibitor and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126, the linker connecting the antibody or antigen binding fragment thereof to the tubulin inhibitor is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with a tubulin inhibitor and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with a tubulin inhibitor and an antibody or antigen-binding fragment or variant thereof that comprises Heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is Monomethyl auristatin E (MMAE), and A is an antibody or antigen-binding fragment or variant thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the MMAE is attached to the antibody or antigen-binding fragment thereof at a lysine residue. In some embodiments, the MMAE is attached to the antibody or antigen-binding fragment thereof at a cysteine residue. In some embodiments, the MMAE is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with MMAE and an antibody or antigen-binding fragment or variant thereof that comprises VL CDRs of SEQ ID NOs: 61, 62, and 63 and VH CDRs of SEQ ID NOs: 58, 59, and 60, the linker connecting the antibody or antigen binding fragment thereof to the MMAE is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with MMAE and an antibody or antigen-binding fragment or variant thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with MMAE and an antibody or antigen-binding fragment or variant thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is MMAE, and A is an antibody or antigen-binding fragment or variant thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the MMAE is attached to the antibody or antigen-binding fragment thereof at a lysine residue. In some embodiments, the MMAE is attached to the antibody or antigen-binding fragment thereof at a cysteine residue. In some embodiments, the MMAE is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with MMAE and an antibody or antigen-binding fragment or variant thereof that comprises VL CDRs of SEQ ID NOs: 9, 10, and 11 and VH CDRs of SEQ ID NOs: 15, 4, and 5, the linker connecting the antibody or antigen binding fragment thereof to the MMAE is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with MMAE and an antibody or antigen-binding fragment or variant thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with MMAE and an antibody or antigen-binding fragment or variant thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is MMAE, and A is an antibody or antigen-binding fragment or variant thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the MMAE is attached to the antibody or antigen-binding fragment thereof at a lysine residue. In some embodiments, the MMAE is attached to the antibody or antigen-binding fragment thereof at a cysteine residue. In some embodiments, the MMAE is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with MMAE and an antibody or antigen-binding fragment or variant thereof that comprises VL CDRs of SEQ ID NOs: 29, 10, and 11 and VH CDRs of SEQ ID NOs: 15, 26, and 5, the linker connecting the antibody or antigen binding fragment thereof to the MMAE is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with MMAE and an antibody or antigen-binding fragment or variant thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with MMAE and an antibody or antigen-binding fragment or variant thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is MMAE, and A is an antibody or antigen-binding fragment or variant thereof that comprises heavy chain variable region (VH) with an amino acid sequence of SEQ ID NO:69 and a light chain variable region (VL) with an amino acid sequence of SEQ ID NO:90. In some embodiments, the MMAE is attached to the antibody or antigen-binding fragment thereof at a lysine residue. In some embodiments, the MMAE is attached to the antibody or antigen-binding fragment thereof at a cysteine residue. In some embodiments, the MMAE is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with MMAE and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, the linker connecting the antibody or antigen binding fragment thereof to the MMAE is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with MMAE and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with MMAE and an antibody or antigen-binding fragment or variant thereof that comprises heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is MMAE, and A is an antibody or antigen-binding fragment or variant thereof that comprises heavy chain variable region (VH) with an amino acid sequence of SEQ ID NO:127 and a light chain variable region (VL) with an amino acid sequence of SEQ ID NO:126. In some embodiments, the MMAE is attached to the antibody or antigen-binding fragment thereof at a lysine residue. In some embodiments, the MMAE is attached to the antibody or antigen-binding fragment thereof at a cysteine residue. In some embodiments, the MMAE is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an ADC with MMAE and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126, the linker connecting the antibody or antigen binding fragment thereof to the MMAE is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an ADC with MMAE and an antibody or antigen-binding fragment or variant thereof that comprises heavy chain variable region (VH) with an amino acid sequence of SEQ ID NO:127 and a light chain variable region (VL) with an amino acid sequence of SEQ ID NO:126 are provided. In some such compositions, the average drug to antibody ratio (DAR) for ADC in the composition at least 2. In some such compositions, the average DAR for ADC in the composition is at least 4. In some such compositions, the average DAR for ADC in the composition is at least 6. In some such compositions, the average DAR for ADC in the composition is at least 8. In some such compositions, the average DAR for ADC in the composition is at least 2, at least 3, at least 4, at least 5, at least 6, at least 7, at least 8, at least 9, at least 10, or greater. In some such compositions, the average DAR for ADC in the composition is at least 6.

In some embodiments, methods are provided for treating cancer by administering, to a subject in need thereof, a therapeutically effective amount of an ADC with MMAE and an antibody or antigen-binding fragment or variant thereof that comprises Heavy chain variable region (VH) with an amino acid sequence of SEQ ID NO:127 and a light chain variable region (VL) with an amino acid sequence of SEQ ID NO:126, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. IV. Pharmaceutical Compositions

In embodiments, ADCs described herein can be used therapeutically in combination with a pharmaceutically acceptable carrier.

In embodiments, the present disclosure provides a pharmaceutical composition comprising an antibody-drug conjugate (ADC) having the formula A-(L-Pr)q, wherein: A is a 3E10 antibody or antigen-binding fragment or variant thereof, L is a linker, P is a payload as described herein, r is an integer from 1 to 4, and q is an integer from 1 to 16; wherein the 3E10 antibody or antigen-binding fragment thereof comprises (a) a light chain variable region (VL) complementarity determining region (CDR) 1 comprising the amino acid sequence of X1ASX2X3VSTSSYSYX4X5, where X1 is K, R, or H, X2 is K, R, or H, X3 is T or S, X4 is M or L, and X5 is K, R, H, or A (SEQ ID NO:61), (b) a VL CDR2 comprising the amino acid sequence of YASYLX1S, where X1 is D, E, N, or Q (SEQ ID NO:62), and (c) a VL CDR3 comprising the amino acid sequence of QX1SX2X3FPWT, where X1 is K, R, or H, X2 is K, R, or H, and X3 is D or E (SEQ ID NO:63), and (d) a heavy chain variable region (VH) CDR1 comprising the amino acid sequence of X1YGMX2, where X1 is D, E, N, Q, R, or K and X2 is K, R, or H (SEQ ID NO:58), (e) a VH CDR2 comprising the amino acid sequence of YISSX1SSTIYYAX2X3VX4G, where X1 is G or S, X2 is D or E, X3 is T or S, and X4 is K, R, or H (SEQ ID NO:59), and (f) a VH CDR3 comprising the amino acid sequence of X1GLLLX2Y, where X1 is K, R, or H, and X2 is D or E (SEQ ID NO:60), and a pharmaceutically acceptable carrier.

In embodiments, the present disclosure provides a pharmaceutical composition comprising an antibody-drug conjugate (ADC) having the formula A-(L-Pr)q, wherein: A is a 3E10 antibody or antigen-binding fragment or variant thereof, L is a linker, P is a payload as described herein, r is an integer from 1 to 4, and q is an integer from 1 to 16; wherein the 3E10 antibody or antigen-binding fragment thereof comprises a heavy chain variable region (VH) CDR1 comprising the amino acid sequence of NYGMH (SEQ ID NO: 15), and a pharmaceutically acceptable carrier. In embodiments, the antibody or antigen-binding fragment or variant thereof comprises: (a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO:10), and (c) a VL CDR3 comprising the amino acid sequence of QHSREFPWT (SEQ ID NO: 11), and (d) a heavy chain variable region (VH) CDR1 comprising the amino acid sequence of NYGMH (SEQ ID NO: 15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5), and a pharmaceutically acceptable carrier. In embodiments, the antibody or antigen-binding fragment or variant thereof comprises a light chain variable region (VL) comprising an amino acid sequence that is identical to SEQ ID NO:21. In embodiments, the antibody or antigen-binding fragment or variant thereof comprises a heavy chain variable region (VH) comprising an amino acid sequence that is identical to SEQ ID NO:14. In embodiments, the antibody or antigen-binding fragment or variant thereof comprises a full length light chain (LC) comprising an amino acid sequence that is identical to SEQ ID NO:20. In embodiments, the antibody or antigen-binding fragment or variant thereof comprises a full length heavy chain (HC) comprising an amino acid sequence that is identical to SEQ ID NO:13.

In embodiments of ADCs provided herein, the amino acid residue corresponding with N31 of the heavy chain CDR1 is substituted with R. In embodiments, the amino acid residue corresponding with N31 of the heavy chain CDR1 is substituted with L. In embodiments, the amino acid residue corresponding with N31 of the heavy chain CDR1 is substituted with K.

In embodiments of pharmaceutical compositions comprising an ADC provided herein, the antibody or antigen-binding fragment or variant thereof is murine, chimeric, humanized, or human.

In embodiments of the pharmaceutical composition comprising an antibody-drug conjugate (ADC) provided herein, L is a cleavable linker. In embodiments, L is selected from the succinyl, 0-succinyl, 4-succinimidyl-oxycarbonyl-α-(2-pyridyldithio)toluene, sulfosuccinimidyl 6-(3′-(2-pyridyldithio)propionamido)hexanoate, N-succinimidyl-3-(-2-pyridyldithio)-proprionate, succinimidyl 6-(3(2-pyridyldithio)propionamido)hexanoate, 3-(2-pyridyldithio)-propionyl hydrazide, S-(2-thiopyridyl)-L-cysteine, N-succinimidyl 4-(2-pyridyldithio)butanoate (SPDB), N-succinimidyl 4-(2-pyridyldithio)-2-sulfobutanoate (sulfo-SPDB), N-succinimidyl 4-(2-pyridyldithio)pentanoate (SPP), CL2A, maleimidocaproyl-valine-citrulline-p-aminobenzoyloxycarbonyl (MC-vc-PAB) and N-succinimidyl 4-(2-pyridyldithio)-2-sulfopentanoate (sulfo-SPP).In embodiments of the pharmaceutical composition comprising an antibody-drug conjugate (ADC) provided herein, L is a non-cleavable linker. In embodiments, L is selected from N-succinimidyl (4-iodoacetyl)-aminobenzoate, sulfosuccinimidyl(4-iodoacetyl)-aminobenzoate, dichlorotriazinic acid, N-succinimidyl-[(N-maleimidopropionamido)-tetraethyleneglycol]ester (NHS-PEG4-maleimide), N-succinimidyl 4-(maleimidomethyl) cyclohexanecarboxylate (SMCC), or N-sulfosuccinimidyl 4-(maleimidomethyl) cyclohexanecarboxylate (sulfoSMCC).

In embodiments of the pharmaceutical composition comprising an antibody-drug conjugate (ADC) provided herein, P is a DNA damage inducing agent, a DNA repair inhibitor, an immune modulatory molecule, an alkylating agent, a microtubule inhibitor, an immune checkpoint inhibitor, an angiogenesis inhibitor, adoptive cell therapy, or a topoisomerase inhibitor. In embodiments, P is an anti-tumor drug selected from maytansinoids, benzodiazepines, auristatins, tecans, taxoids, CC-1065, (4S)-4,11-Diethyl-4,9-dihydroxy-1,4-dihydro-3H,14H-pyrano[3′,4′:6,7]indolizino[1,2-b]quinoline-3,14-dione (SN38), exatecan, monomethyl auristatin E (MMAE), monomethyl auristatin F (MMAF), pyrrolobenzodiazepines (PBDs), PROteolysis TArgeting Chimera (PROTAC), deruxtecan (Dxd), calicheamicins, duocarmycins, stimulator of interferon genes (STING) agonists, PNU-159682, NMS249, IMGN Camp 1, duocarmycin hydroxybenzamide azaindole (DUBA) and a prodrug anti-tumor drug thereof. In embodiments, P is a maytansinoid. In embodiments, P is N(2′)-deacetyl-N(2′)-(3-mercapto-1-oxopropyl)-maytansine (DM1). In embodiments, P is N2′-deacetyl-N2′-(4-mercapto-4-methyl-1-oxopentyl) maytansine (DM4). In embodiments, P is PNU-159682. In embodiments, P is NMS249.

In embodiments of the pharmaceutical composition comprising an antibody-drug conjugate (ADC) provided herein, P is (4S)-4,11-Diethyl-4,9-dihydroxy-1,4-dihydro-3H,14H-pyrano[3′,4′:6,7]indolizino[1,2-b]quinoline-3,14-dione (SN38).

In embodiments of the pharmaceutical composition comprising an antibody-drug conjugate (ADC) provided herein, A is the 3E10 antibody or antigen-binding fragment or variant described herein, L is SMCC, and P is DM1.

In embodiments of the pharmaceutical composition comprising an antibody-drug conjugate (ADC) provided herein, A is the 3E10 antibody or antigen-binding fragment or variant described herein, L is O-Succinyl and P is SN38.

In embodiments of the pharmaceutical composition comprising an antibody-drug conjugate (ADC) provided herein A is a 3E10 antibody or antigen-binding fragment or variant thereof, L is CL2A and P is SN38.

In embodiments of the pharmaceutical composition comprising an antibody-drug conjugate (ADC) provided herein A is a 3E10 antibody or antigen-binding fragment or variant thereof, L is MC-vc-PAB and P is PNU-159682.

In embodiments of the pharmaceutical composition comprising an antibody-drug conjugate (ADC) provided herein A is a 3E10 antibody or antigen-binding fragment or variant thereof, L is MC-vc-PAB and P is NMS249.

In embodiments, the present disclosure provides a pharmaceutical composition comprising an antibody-drug conjugate (ADC) having the formula A-(L-Pr)q, wherein: A is a humanized 3E10 antibody or antigen-binding fragment thereof comprising: (a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO:5), and (c) a VL CDR3 comprising the amino acid sequence of QHSREFPWT (SEQ ID NO:11), and (d) a heavy chain variable region (VH) CDR1 comprising the amino acid sequence of NYGMH (SEQ ID NO:15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5), L is O-Succinyl, and P is SN38.

In embodiments, the present disclosure provides a pharmaceutical composition comprising an ADC disclosed herein that has the formula A-(L-Pr)q, wherein: A is a 3E10 antibody or antigen-binding fragment or variant thereof, L is a linker, P is a payload as described herein, r is an integer from 1 to 4, and q is an integer from 1 to 16; wherein the 3E10 antibody or antigen-binding fragment thereof comprises a heavy chain variable region (VH) CDR1 comprising the amino acid sequence of XYGMH (SEQ ID NO: 16), wherein X=D, N, R, L, or K and corresponds to an amino acid residue at position 31 of the 3E10 heavy chain, and wherein D31 is wild-type 3E10 CDR1. A. Pharmaceutically Acceptable Carriers

The pharmaceutical composition can be in solution, emulsions, or in a suspension (for example, incorporated into microparticles, liposomes, or cells). An appropriate amount of a pharmaceutically-acceptable salt can be used in the formulation to render the formulation isotonic. Examples of pharmaceutically-acceptable carriers include, but are not limited to, saline, Ringer's solution and dextrose solution. The pH of the solution can be from about 5 to about 8, or from about 7 to about 7.5. Pharmaceutical compositions can include carriers, thickeners, diluents, buffers, preservatives, and surface active agents. Further carriers include sustained release preparations such as semi-permeable matrices of solid hydrophobic polymers containing the antibody, which matrices are in the form of shaped particles, e.g., films, liposomes or microparticles. It will be apparent to those persons skilled in the art that certain carriers can be used, depending upon, for instance, the route of administration and concentration of composition being administered. Pharmaceutical compositions can also include one or more active ingredients such as antimicrobial agents, anti-inflammatory agents, and anesthetics.

Preparations for parenteral administration include sterile aqueous or non-aqueous solutions, suspensions, and emulsions. Parenteral vehicles include sodium chloride solution, Ringer's dextrose, dextrose and sodium chloride, lactated Ringer's, or fixed oils. Preservatives and other additives can also be present such as, for example, antimicrobials, anti-oxidants, chelating agents, and inert gases.

To aid dissolution of antibodies into the aqueous environment a surfactant might be added as a wetting agent. Surfactants can include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios. Additives which potentially enhance uptake of peptides are for instance the fatty acids oleic acid, linoleic acid and linolenic acid.

In certain aspects, the compositions disclosed herein are formulated with additional agents that promote entry into the desired cell or tissue. Such additional agents include micelles, liposomes, and dendrimers. B. Dosages

The term “effective amount” refers an amount that is non-toxic to a subject or a majority or normal cells, but is an amount of the active agent that is sufficient to provide a desired effect. This amount can vary from subject to subject, depending on the species, age, and physical condition of the subject, the severity of the disease that is being treated, the particular conjugate, or more specifically, the particular active agent used, its mode of administration, and the like.

The term “pharmaceutically acceptable” refers to the fact that the carrier, diluent or excipient must be compatible with the other ingredients of the formulation and not deleterious to the recipient thereof. For example, the carrier, diluent, or excipient or composition thereof can be administered to a subject along with an antibody-drug conjugate (ADC) of the disclosure without causing any undesirable biological effects or interacting in an undesirable manner with any of the other components of the pharmaceutical composition in which it is contained. Pharmaceutical compositions including an ADC can be administered by any suitable means, for example, parenterally, such as by Subcutaneous, intravenous, intramuscular, intrathecal, or intracisternal injection or infusion techniques (e.g., as sterile injectable aqueous or non-aqueous Solutions or Suspensions) in dosage formulations containing non-toxic, pharmaceutically acceptable vehicles or diluents. In embodiments the conjugate is administered parenterally, or intravenously.

The pharmaceutical compositions of an ADC described herein can be administered either alone or in combination with other therapeutic agents, can conveniently be presented in unit dose form and can be prepared by any of the methods well known in the art of pharmacy. All methods include bringing the conjugate into association with the carrier, which constitutes one or more accessory ingredients, are contemplated. Techniques for formulation and administration are generally known in the art. Suitable routes can be, for example, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural, intrasternal, topical, epidermal, or mucosal administration. V. Methods of Use

In embodiments, the present disclosure provides methods of treating cancer. The compositions, conjugates, and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

In embodiments, the present disclosure provides a method of treating cancer in a subject in need thereof, comprising administering a therapeutically effective amount of a pharmaceutical composition comprising an antibody-drug conjugate (ADC) having the formula A-(L-Pr)q, wherein: A is a 3E10 antibody or antigen-binding fragment or variant thereof, L is a linker, P is a payload as described herein, r is an integer from 1 to 4, and q is an integer from 1 to 16; and wherein the 3E10 antibody or antigen-binding fragment thereof comprises a heavy chain variable region (VH) CDR1 comprising the amino acid sequence of NYGMH (SEQ ID NO: 15). In embodiments, the antibody or antigen-binding fragment or variant thereof comprises (a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO:10), and (c) a VL CDR3 comprising the amino acid sequence of QHSREFPWT (SEQ ID NO: 11), and (d) a heavy chain variable region (VH) CDR1 comprising the amino acid sequence of NYGMH (SEQ ID NO: 15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5).

In embodiments, the present disclosure provides a method of inducing death of a cancer cell in a subject in need thereof, comprising administering a therapeutically effective amount of a pharmaceutical composition comprising an antibody-drug conjugate (ADC) having the formula A-(L-Pr)q, wherein: A is a 3E10 antibody or antigen-binding fragment or variant thereof, L is a linker, P is a payload as described herein, r is an integer from 1 to 4, and q is an integer from 1 to 16; and wherein the 3E10 antibody or antigen-binding fragment thereof comprises a heavy chain variable region (VH) CDR1 comprising the amino acid sequence of NYGMH (SEQ ID NO: 15). In embodiments, the antibody or antigen-binding fragment or variant thereof comprises: (a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO:10), and (c) a VL CDR3 comprising the amino acid sequence of QHSREFPWT (SEQ ID NO: 11), and (d) a heavy chain variable region (VH) CDR1 comprising the amino acid sequence of NYGMH (SEQ ID NO: 15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5).

In embodiments of the methods disclosed herein, the antibody or antigen-binding fragment or variant thereof comprises a light chain variable region (VL) comprising an amino acid sequence that is identical to SEQ ID NO:21. In embodiments, the antibody or antigen-binding fragment or variant thereof comprises a heavy chain variable region (VH) comprising an amino acid sequence that is identical to SEQ ID NO:14. In embodiments, the antibody or antigen-binding fragment or variant thereof comprises a full length light chain (LC) comprising an amino acid sequence that is identical to SEQ ID NO:20. In embodiments, the antibody or antigen-binding fragment or variant thereof comprises a full length heavy chain (HC) comprising an amino acid sequence that is identical to SEQ ID NO:13.

In embodiments of the methods disclosed herein, the amino acid residue corresponding with N31 of the heavy chain CDR1 is substituted with R. In embodiments, the amino acid residue corresponding with N31 of the heavy chain CDR1 is substituted with L. In embodiments, the amino acid residue corresponding with N31 of the heavy chain CDR1 is substituted with K.

In embodiments of the methods disclosed herein, the antibody or antigen-binding fragment or variant thereof is murine, chimeric, humanized, or human.

In embodiments of the methods disclosed herein, L is a cleavable linker. In embodiments, L is selected from succinyl, 0-succinyl, 4-succinimidyl-oxycarbonyl-α-(2-pyridyldithio)toluene, sulfosuccinimidyl 6-(3′-(2-pyridyldithio)propionamido)hexanoate, N-succinimidyl-3-(-2-pyridyldithio)-proprionate, succinimidyl 6-(3(2-pyridyldithio)propionamido)hexanoate, 3-(2-pyridyldithio)-propionyl hydrazide, S-(2-thiopyridyl)-L-cysteine, N-succinimidyl 4-(2-pyridyldithio)butanoate (SPDB), N-succinimidyl 4-(2-pyridyldithio)-2-sulfobutanoate (sulfo-SPDB), N-succinimidyl 4-(2-pyridyldithio)pentanoate (SPP), CL2A, maleimidocaproyl-valine-citrulline-p-aminobenzoyloxycarbonyl (MC-vc-PAB) and N-succinimidyl 4-(2-pyridyldithio)-2-sulfopentanoate (sulfo-SPP).

In embodiments of the methods disclosed herein, L is a non-cleavable linker. In embodiments, L is selected from N-succinimidyl (4-iodoacetyl)-aminobenzoate, sulfosuccinimidyl(4-iodoacetyl)-aminobenzoate, dichlorotriazinic acid, N-succinimidyl-[(N-maleimidopropionamido)-tetraethyleneglycol]ester (NHS-PEG4-maleimide), N-succinimidyl 4-(maleimidomethyl) cyclohexanecarboxylate (SMCC), or N-sulfosuccinimidyl 4-(maleimidomethyl) cyclohexanecarboxylate (sulfoSMCC).

In embodiments of the methods disclosed herein, P is a DNA damage inducing agent, a DNA repair inhibitor, an immune modulatory molecule, an alkylating agent, a microtubule inhibitor, an immune checkpoint inhibitor, an angiogenesis inhibitor, adoptive cell therapy, or a topoisomerase inhibitor. In embodiments, of the methods disclosed herein, P is an anti-tumor drug selected from maytansinoids, benzodiazepines, auristatins, tecans, taxoids, CC-1065, (4S)-4,11-Diethyl-4,9-dihydroxy-1,4-dihydro-3H,14H-pyrano[3′,4′:6,7]indolizino[1,2-b]quinoline-3,14-dione (SN38), exatecan, monomethyl auristatin E (MMAE), monomethyl auristatin F (MMAF), pyrrolobenzodiazepines (PBDs), PROteolysis TArgeting Chimera (PROTAC), deruxtecan (Dxd), calicheamicins, duocarmycins, stimulator of interferon genes (STING) agonists, PNU-159682, NMS249, IMGN Camp 1, duocarmycin hydroxybenzamide azaindole (DUBA) and a prodrug anti-tumor drug thereof.

In embodiments of the methods disclosed herein, P is a maytansinoid. In embodiments, P is N2′-deacetyl-N2′-(3-mercapto-1-oxopropyl)-maytansine (DM1). In embodiments, P is N2′-deacetyl-N2′-(4-mercapto-4-methyl-1-oxopentyl) maytansine (DM4).

In embodiments of the methods disclosed herein, P is (4S)-4,11-Diethyl-4,9-dihydroxy-1,4-dihydro-3H,14H-pyrano[3′,4′:6,7]indolizino[1,2-b]quinoline-3,14-dione (SN38).

In embodiments of the methods disclosed herein, P is PNU-159682. In embodiments of the methods disclosed herein, P is NMS249.

In embodiments of the methods disclosed herein, A is a 3E10 antibody or antigen-binding fragment or variant thereof described herein, L is SMCC, and P is DM1.

In embodiments of the methods disclosed herein, A is a 3E10 antibody or antigen-binding fragment or variant thereof described herein, L is O-Succinyl, and P is SN38.

In embodiments of the methods disclosed herein, A is a 3E10 antibody or antigen-binding fragment or variant thereof, L is CL2A and P is SN38.

In embodiments of the methods disclosed herein, A is a 3E10 antibody or antigen-binding fragment or variant thereof, L is MC-vc-PAB and P is PNU-159682.

In embodiments of the methods disclosed herein, A is a 3E10 antibody or antigen-binding fragment or variant thereof, L is MC-vc-PAB and P is NMS249.

In embodiments, the present disclosure provides a method of treating cancer in a subject in need thereof, comprising administering a therapeutically effective amount of a pharmaceutical composition comprising an antibody-drug conjugate (ADC) having the formula A-(L-Pr)q, wherein: A is a humanized 3E10-D31N antibody or antigen-binding fragment thereof comprising: (a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO:10), and (c) a VL CDR3 comprising the amino acid sequence of QHSREFPWT (SEQ ID NO: 11), and (d) a heavy chain variable region (VH) CDR1 comprising the amino acid sequence of NYGMH (SEQ ID NO: 15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5), L is O-Succinyl, and P is SN38.

In embodiments, the present disclosure provides a method of treating cancer in a subject in need thereof, comprising administering a therapeutically effective amount of a pharmaceutical composition comprising an ADC disclosed herein that has the formula A-(L-Pr)q, wherein: A is a 3E10 antibody or antigen-binding fragment or variant thereof, L is a linker, P is a payload as described herein, r is an integer from 1 to 4, and q is an integer from 1 to 16; wherein the 3E10 antibody or antigen-binding fragment thereof comprises a heavy chain variable region (VH) CDR1 comprising the amino acid sequence of XYGMH (SEQ ID NO: 16), wherein X=D, N, R, L, or K and corresponds to an amino acid residue at position 31 of the 3E10 heavy chain, and wherein D31 is wild-type 3E10 CDR1. A. Cancers

In embodiments, the subject has a cancer selected from colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

Examples of cancer can also include small-cell lung cancer (SCLC), non-small cell lung cancer (NSCLC), squamous NSCLC, nonsquamous NSCLC, clear cell carcinoma, endometrial cancer, renal cell carcinoma (RCC), hormone refractory prostate adenocarcinoma, glioblastoma multiforme, stomach cancer, hepatoma (hepatocellular carcinoma), colon carcinoma, head and neck squamous cell carcinoma (HNSCC), germ cell tumor, pediatric sarcoma, sinonasal natural killer, metastatic malignant melanoma, cutaneous or intraocular malignant melanoma, mesothelioma, skin cancer, cancer of the anal region, testicular cancer, carcinoma of the fallopian tubes, carcinoma of the endometrium, carcinoma of the cervix, carcinoma of the vagina, carcinoma of the vulva, cancer of the small intestine, cancer of the parathyroid gland, cancer of the adrenal gland, sarcoma of soft tissue, cancer of the urethra, cancer of the penis, cancer of the ureter, carcinoma of the renal pelvis, neoplasm of the central nervous system (CNS), spinal axis tumor, pituitary adenoma, Kaposi's sarcoma, epidermoid cancer, squamous cell cancer, environmentally-induced cancers including those induced by asbestos, virus-related cancers or cancers of viral origin, human papilloma virus (HPV)-related or -originating tumors, and any combinations of said cancers.

Examples of cancer can also include acute leukemia (ALL), acute myelogenous leukemia (AML), chronic myelogenous leukemia (CML), undifferentiated AML, myeloblastic leukemia, promyelocytic leukemia, myelomonocytic leukemia, monocytic leukemia, erythroleukemia, megakaryoblastic leukemia, isolated granulocytic sarcoma, chloroma B-cell lymphoma, T-cell lymphoma, lymphoplasmacytoid lymphoma, monocytoid B-cell lymphoma, mucosa-associated lymphoid tissue (MALT) lymphoma, anaplastic large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic lymphoma, peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, lymphoplasmacytoid lymphoma (LPL) with Waldenstrom's macroglobulinemia, myeloma, Ig1 myeloma, light chain myeloma, nonsecretory myeloma, smoldering myeloma (indolent myeloma), solitary plasmocytoma, multiple myeloma, hairy cell lymphoma, and any combinations of said cancers.

Malignant tumors which can be treated can be classified according to the embryonic origin of the tissue from which the tumor is derived. Carcinomas are tumors arising from endodermal or ectodermal tissues such as skin or the epithelial lining of internal organs and glands. The disclosed compositions can be effective in treating carcinomas. Sarcoma, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors can show up at numerous organs or tissues of the body to establish a cancer.

In embodiments of the methods of the present disclosure, the administration of an ADC induces cell killing that is measurable by a tumor cell-killing potency (EC50) value. In embodiments, administration of an ADC induces cell killing in a breast cancer cell line with an EC50 value of from about 0.2-13 nM. In embodiments, administration of an ADC induces cell killing in a colorectal cancer cell line with an EC50 value of from about 50-90 nM. In embodiments, administration of an ADC induces cell killing in a colorectal cancer cell line with an EC50 value of from about 1-3 nM. In embodiments, administration of an ADC induces cell killing in an ovarian cancer cell line with an EC50 value of from about 2-20 nM. In embodiments, administration of an ADC induces cell killing with an EC50 value of from about 2.5-9.5 nM. In embodiments, administration of an ADC induces cell killing with an EC50 value of from about 50-90 nM.

In some embodiments, the disclosure provides a method for treating cancer in a subject in need thereof, the method comprising administering to the subject a therapeutically effective amount of a conjugate described herein to the subject.

In some embodiments, the cancer is a carcinoma, a sarcoma, a blastoma, a papilloma, or an adenoma. In some embodiments, the cancer is metastatic cancer.

In some embodiments, the cancer is selected from the group consisting of bladder cancer, blood cancer, brain cancer, breast cancer, bone cancer, cervical cancer, colorectal cancer, endocrine cancer, esophageal cancer, gastric cancer, head and neck cancer, hepatobiliary cancer, leukemia, lung cancer, lymphoma, melanoma, myeloma, ovarian cancer, pancreatic cancer, prostate cancer, renal cancer, thyroid cancer, and uterine cancer.

In some embodiments, the cancer is lung cancer. In some embodiments, the lung cancer is small-cell lung cancer (SCLC), non-small cell lung cancer (NSCLC), squamous NSCLC, or nonsquamous NSCLC. In some embodiments, the lung cancer is non-small cell lung cancer (NSCLC).

In some embodiments, the cancer is breast cancer. In some embodiments, the cancer is BRCA2 negative breast cancer. In some embodiments, the cancer is triple negative breast cancer.

In some embodiments, the cancer is a skin cancer selected from the group consisting of basal cell carcinoma, squamous cell carcinoma, and melanoma. In some embodiments, the cancer is melanoma.

In some embodiments, the cancer is a cancer of the central nervous system. In some embodiments, the cancer is a neuroepithelial brain or spinal tumor selected from the group consisting of a medulloblastoma, an astrocytic tumor, an oligodendroglial tumor, an oligoastrocytic tumor, an ependymal tumor, a choroid plexus tumor, a neuronal or mixed neuronal-glial tumor, a tumor of the pineal region, an embryonal tumor, or an otherwise uncategorized neuroepithelial tumor.

In some embodiments, the administering is by parenteral administration. In some embodiments, the parenteral administration is intramuscular administration, intravenous administration, or subcutaneous administration. B. Dosages and Administration

Administration of the suitable compositions provided herein can be effected by different ways, e.g., by intravenous, intraperitoneal, subcutaneous, intramuscular, intrathecal, intratumoral, intraocular, intravitreal, intraarterial, topical, intradermal, etc., administration. The route of administration, of course, depends, inter alia, on the ADC contained in the pharmaceutical composition.

The dosage regimen will be determined by the attending physician and other clinical factors. As is well known in the medical arts, dosages for any one patient depends on many factors, including the patient's size, body surface area, age, sex, the particular compound to be administered, time and route of administration, the kind and stage of infection or disease, general health and other drugs being administered concurrently. C. Uses

Uses of an ADC described herein are also contemplated. In embodiments, the present disclosure provides a use of an ADC described herein, or the pharmaceutical composition comprising an ADC described herein, for the treatment of cancer in a subject in need thereof. In embodiments, the present disclosure provides a use of an ADC described herein, or the pharmaceutical composition comprising an ADC described herein, for inducing death of a cancer cell in a subject in need thereof. In embodiments, the present disclosure provides a use of an ADC described herein, or the pharmaceutical composition comprising an ADC described herein, for inhibiting tumor growth in a subject in need thereof.

### Examples

### Example 1: Preparation of a 3E10-D31N-DM1 Conjugate

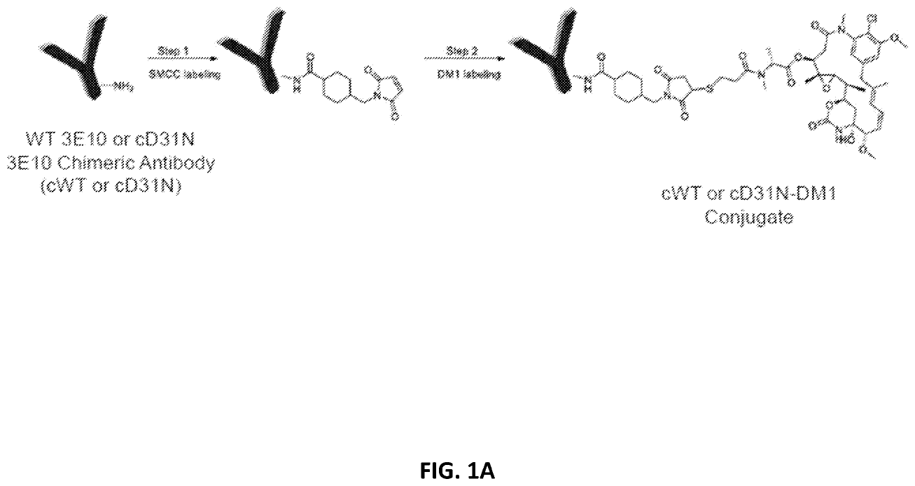

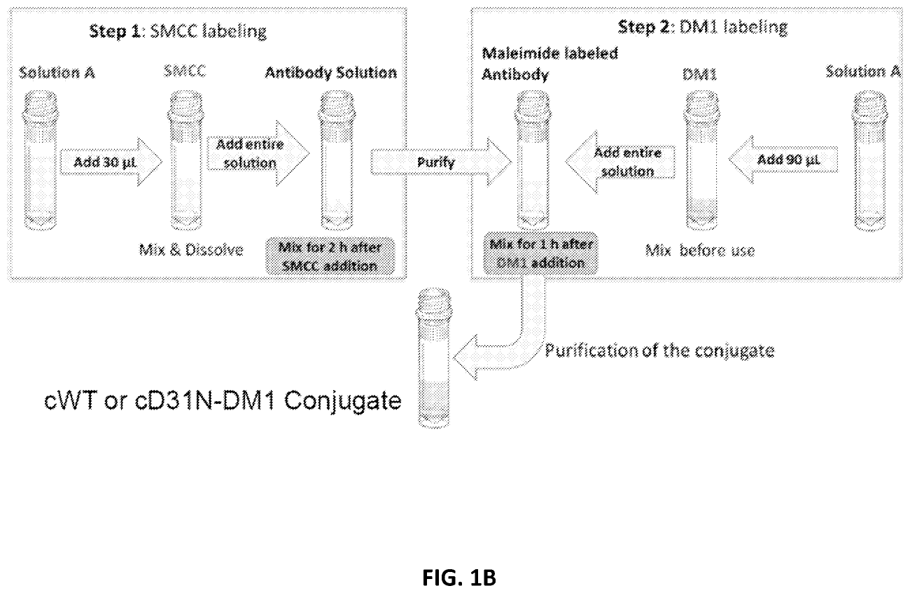

A chimeric 3E10-D31N antibody (cD31N) was conjugated to Maytansinoid DM1 (N2′-deacetyl-N2′-(3-mercapto-1-oxopropyl)-maytansine) (DM1) for testing. The synthesis of the cD31N-DM1 antibody-drug conjugate (ADC) is shown in FIG. 1A. In step 1, the 3E10-cD31N antibody was labeled with a succinimidyl 4-(N-maleimidomethyl)cyclohexane-1-carboxylate (SMCC) linker by mixing the 3E10-cD31N antibody and SMCC linker for 2 hours. In step 2, DM1 was conjugated to the SMCC linker by mixing the labeled antibody solution with the DM1 solution for 1 hour. Subsequently, cD31N-DM1 conjugate is purified. The conjugation process is also shown in FIG. 1B. This general process can be used to synthesis antibody-drug conjugates comprising the various 3E10 antibodies, linkerscytotoxic agents described herein.

### Example 2: cD31N-DM1 Characterization

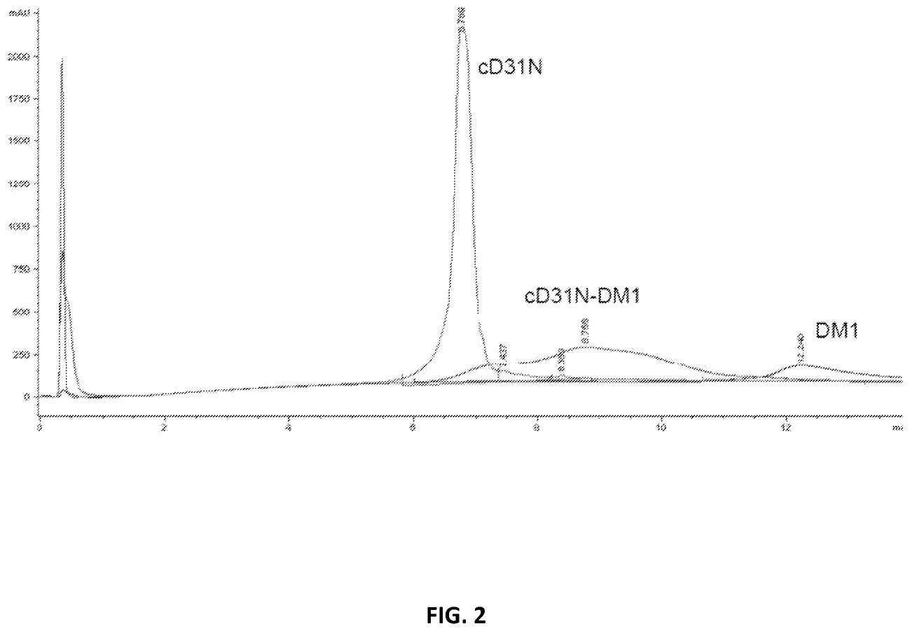

The stability of the cD31N-DM1 conjugate was measured by high-performance liquid chromatography (HPLC). After a 14 day incubation at 4° C., cD31N-DM1 conjugate, 3E10-cD31N antibody, and DM1 were each detected in the sample. The HPLC results are shown in the chromatogram of FIG. 2 and show the profile of the conjugate relative to antibody and free payload.

### Example 3: Enhanced Cytotoxicity of the cD31N-DM1 Conjugate

DM1 is a DNA-damaging agent and is cytoxic to cancer cells. Therefore, to test the efficacy of the cD31N-DM1 conjugate, the cytotoxicity of the cD31N-DM1 conjugates was measured in various cancer cell types.

Cancer cell lines were acquired from ATCC and cultured according to well known methods in the art. Treatment with cytotoxic agents, antibodies, or ADCs refers to the addition of the cytotoxic agent, antibody or ADC (i.e., DM1 or cD31N-DM1, etc.) to the culture media for a set duration.

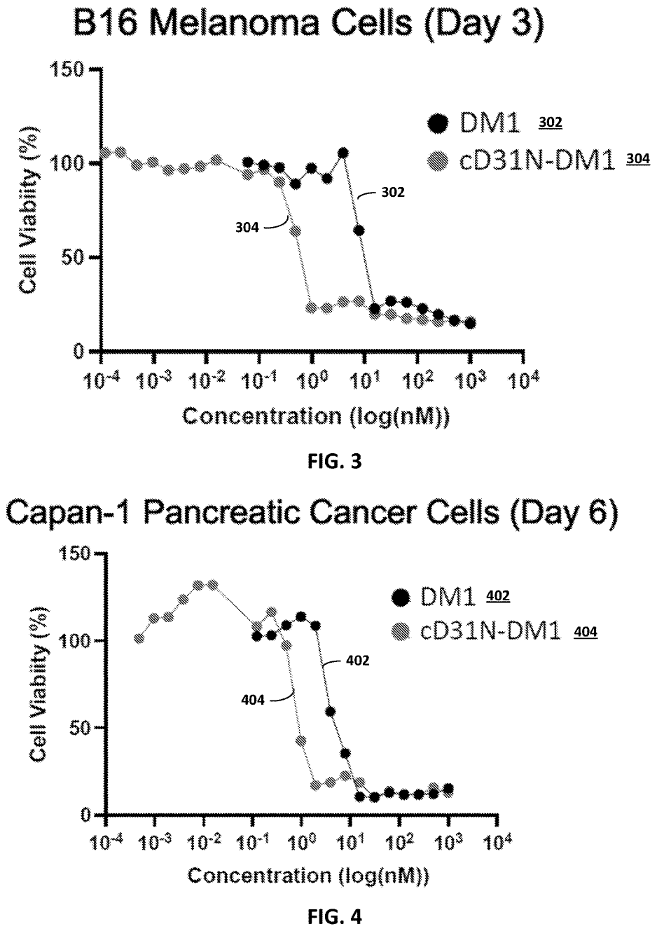

B16 melanoma cells were cultured with increasing concentrations (0.0001 nm to 1000 nm) of DM1 alone or cD31N-DM1 conjugate and cell viability were measured after 3 days. FIG. 3 shows that DM1 alone and cD31N-DM1 conjugate were both cytotoxic to B16 melanoma cells in a concentration-dependent manner. As shown, the cD31N-DM1 conjugate induced a cytoxic effect at lower concentrations compared to DM1 alone. This indicates that conjugating DM1 to cD31N enhances the cytoxicity of DM1 in melanoma cells and therefore the cD31N-DM1 conjugate is more potent.

Capan-1 pancreatic cancer cells were cultured with increasing concentrations of DM1 alone or cD31N-DM1 conjugate and cell viability was measured after 6 days. FIG. 4 shows that treatment with DM1 alone and cD31N-DM1 conjugate were both cytotoxic to the Capan-1 pancreatic cancer cells in a concentration-dependent manner. Importantly, the cD31-DM1 conjugate induced a cytoxic effect at lower concentrations compared to DM1 alone. This indicates that conjugating DM1 to cD31N enhances the cytoxicity of DM1 in pancreatic cancer cells and therefore the cD31N-DM1 conjugate is more potent than DM1 alone.

DAYO medulloblastoma cells were cultured with increasing concentrations of DM1 alone or cD31N-DM1 conjugate and cell viability was measured after 3 days. FIG. 5 shows that DM1 alone and cD31N-DM1 conjugate were both cytotoxic to the DAYO medulloblastoma cells in a concentration-dependent manner. Importantly, cD31N-DM1 conjugate induced a cytoxic effect at lower concentrations compared to DM1 alone. This indicates that conjugating DM1 to cD31N enhances the cytoxicity of DM1 in medulloblastoma cells and therefore the cD31N-DM1 conjugate is more potent than DM1 alone.

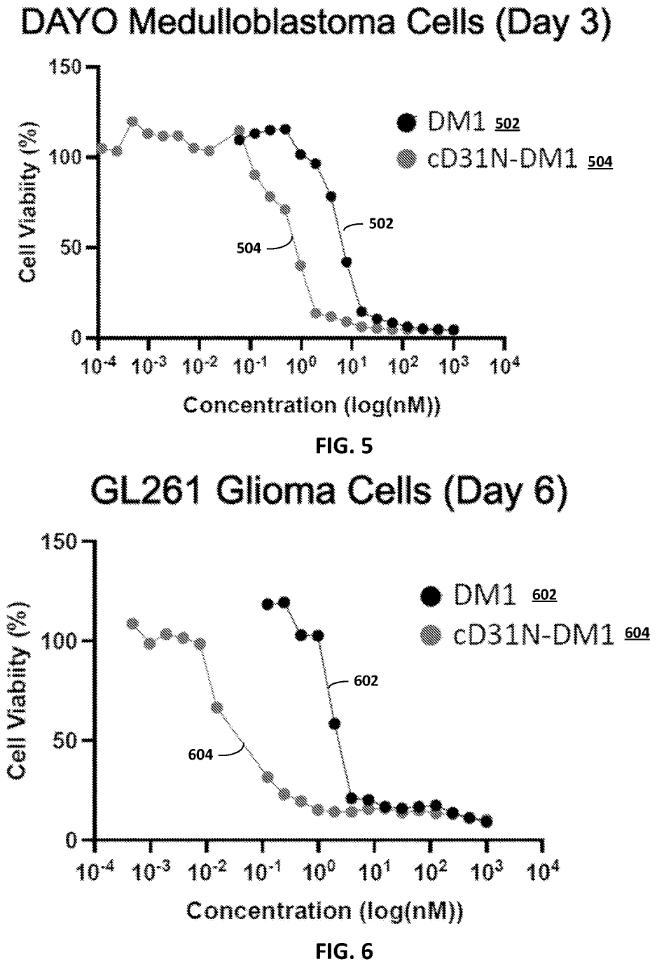

GL261 glioma cells were cultured with increasing concentrations of DM1 alone or cD31N-DM1 conjugate and cell viability was measured after 6 days. FIG. 6 shows that DM1 alone and cD31N-DM1 conjugate were both cytotoxic to the GL261 glioma cells in a concentration-dependent manner. Importantly, the cD31N-DM1 conjugate induced a cytoxic effect at lower concentrations compared to DM1 alone. This indicates that conjugating DM1 to cD31N enhances the cytoxicity of DM1 in glioma cells and therefore the cD31N-DM1 conjugate is more potent than DM1 alone.

DLD-1 colorectal adenocarcinoma cells were cultured with increasing concentrations of DM1 alone or cD31N-DM1 conjugate and cell viability was measured after 6 days. BRCA2 deficient (BRCA2′) cells are more sensitive to DM1 because BRCA2 participates in DNA double-stranded break (DSB) repair. Therefore, the potency of DM1 alone and cD31N-DM1 conjugate was measured in wild-type BRCA2 (WT or BRCA2^{wt/wt}) and BRCA2^{-/-} DLD-1 colorectal adenocarcinoma cells.

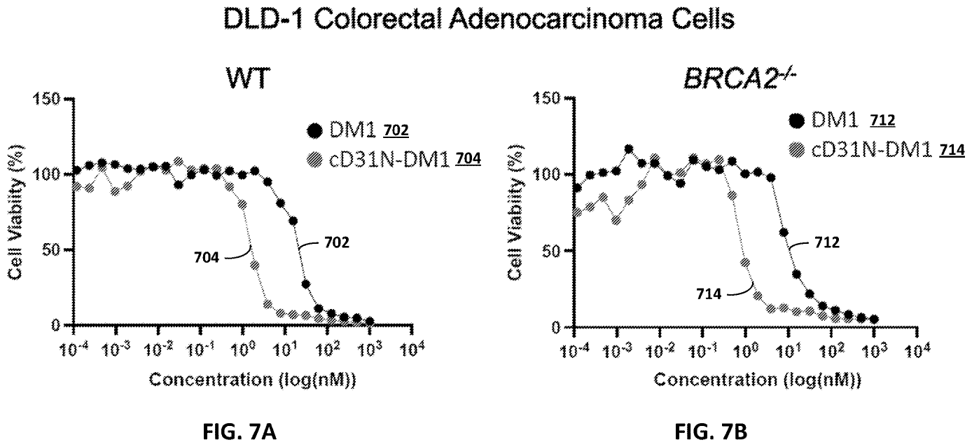

FIGS. 7A and 7B show that DM1 alone and cD31N-DM1 conjugate were cytotoxic to both WT (FIG. 7A) and BRCA2^{-/-} (FIG. 7B) DLD-1 cells in a concentration-dependent manner. Importantly, the cD31N-DM1 conjugate induced a cytoxic effect at lower concentrations compared to DM1 alone. This indicates that conjugating DM1 to cD31N enhances the cytoxicity of DM1 in colorectal adenocarcinoma cells and therefore the cD31N-DM1 conjugate is more potent.

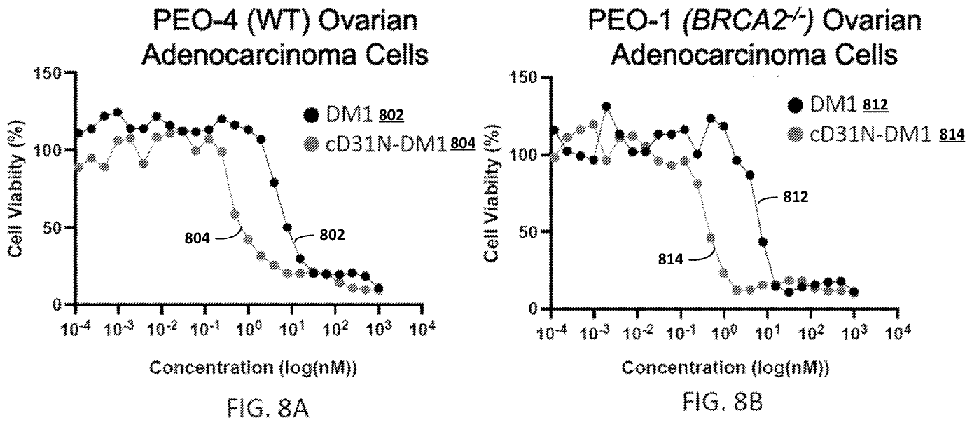

PEO-1 and PEO-4 ovarian adenocarcinoma cells were cultured with increasing concentrations of DM1 alone or cD31N-DM1 conjugate and cell viability was measured after 5 days. The potency of DM1 alone and cD31N-DM1 conjugate was measured in WT PEO-4 and BRCA2/PEO-1 ovarian adenocarcinoma cells. FIGS. 8A and 8B show that DM1 alone and cD31N-DM1 conjugate were cytotoxic to both WT PEO-4 cells (FIG. 8A) and BRCA2^{-/-} PEO-1 cells (FIG. 8B) cells in a concentration-dependent manner. Importantly, cD31N-DM1 conjugate induced a cytoxic effect at lower concentrations compared to DM1 alone. This indicates that conjugating DM1 to cD31N enhances the cytotoxicity of DM1 in ovarian adenocarcinoma cells and therefore the cD31N-DM1 conjugate is more potent.

Based on the results of treatment with DM1 alone or cD31N-DM1 in WT and BRCA2^{-/-}- DLD-1, PEO-4, and PEO-1 cells, both free DM1 and cD31N-DM1 conjugate are cytotoxic regardless of BRCA2 mutation status, and the cD31N-DM1 conjugate was more cytotoxic in both genetic backgrounds.

This example demonstrates the broad applicability and potency of the cD31N-DM1 conjugate against various cancer cell lines. The cD31N-DM1 ADC was effective in all tested cancer cell lines, regardless of genetic background or tissue of origin and the ADC-drug was more potent than free drug in culture in each cell line. This suggests that targeting of the 3E10 ADC is not limited to a specific cancer antigen or tumor epitope. Advantageously, the 3E10 ADC provided herein can widely target any cancer based on cell surface expression of ENT2, a nucleoside transporter overexpressed in many cancers.

### Example 4: Preparation of 3E10-SN38 Conjugates

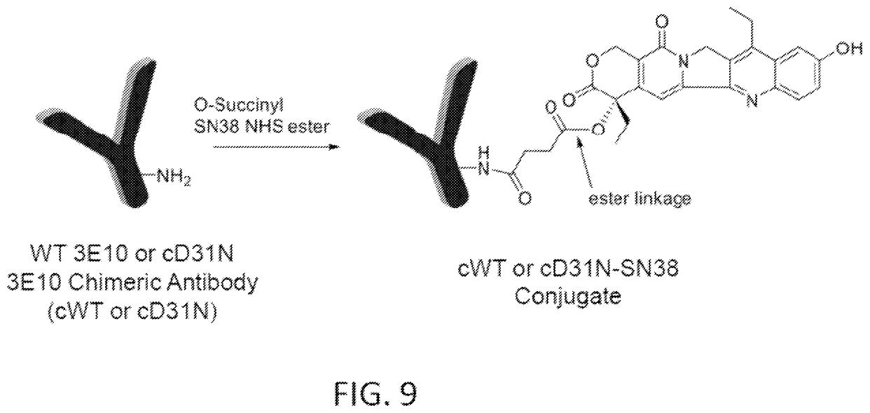

Given the stability and enhanced potency of the 3E10 antibody-drug conjugate with DNA-damaging agent DM1, 3E10 ADCs with the topoisomerase I inhibitor SN38 ((4S)-4,11-Diethyl-4,9-dihydroxy-1,4-dihydro-3H,14H-pyrano[3′,4′:6,7]indolizino[1,2-b]quinoline-3,14-dione) were developed for testing. SN38 was conjugated to either wild-type (cWT-SN38) or D31N (cD31N-SN38) 3E10 chimeric antibodies using the method described in Example 1 and is shown in FIG. 9. The 3E10 antibody was linked to SN38 via an O-Succinyl cleavable linker with an NHS ester linkage.

### Example 5: SN38 and 3E10-SN38 Conjugates Induce DNA Damage and Cell Death in Vitro

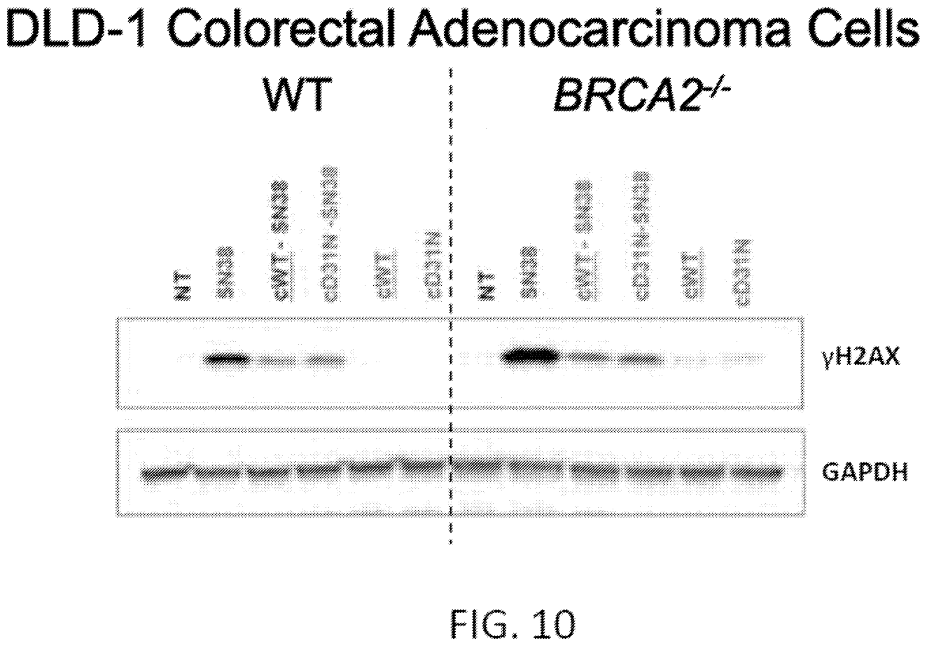

SN38 causes DNA damage via inhibition of topoisomerase I. Therefore, DNA damage was measured in both wild-type (WT) and BRCA2 deficient (BRCA2′) DLD-1 cells by induction of γH2AX after treatment with one of the following: no treatment (NT), SN38 alone (SN38), wild-type 3E10 antibody alone (cWT), 3E10-cD31N antibody alone, cWT-SN38 conjugate, or cD31N-SN38 conjugate. After treatment with 100 nM of the antibody or conjugate for 48 hours, the DLD-1 cells were collected for Western blot analysis to measure 7H2AX protein levels. As shown in FIG. 10, SN38 alone, cWT-SN38 conjugate, and cD31N-SN38 conjugate all induced γH2AX in DLD-1 cells. Treatment with SN38 alone induced the highest levels of γH2AX expression while both SN38 conjugates induced similar, but lower levels of γH2AX. These findings were consistent in both WT and BRCA2^{-/-} DLD-1 cells, but γH2AX protein levels were elevated across all groups in BRCA2^{-/-} DLD-1 cells. Importantly, γH2AX was undetectable in WT DLD-1 cells and only low levels of γH2AX were detected in BRCA2^{-/-} DLD-1 cells after treatment with vehicle control (NT), WT 3E10 antibodies, or 3E10-cD31N antibodies.

These results indicate that SN38 causes DNA damage and that 3E10 antibodies do not induce substantial DNA damage on their own. Therefore, this data demonstrates that the cytotoxic drug (here, SN38) is the primary mediator of DNA damage in 3E10 antibody-drug conjugates and that the antibody itself has little effect on inducing DNA damage.

As demonstrated above, SN38 and 3E10-SN38 conjugates caused DNA damage in DLD-1 colorectal cells. Therefore the cytoxic effect of SN38 and 3E10-SN38 conjugates in wild-type and BRCA2^{-/-} DLD-1 colorectal cancer cells was investigated.

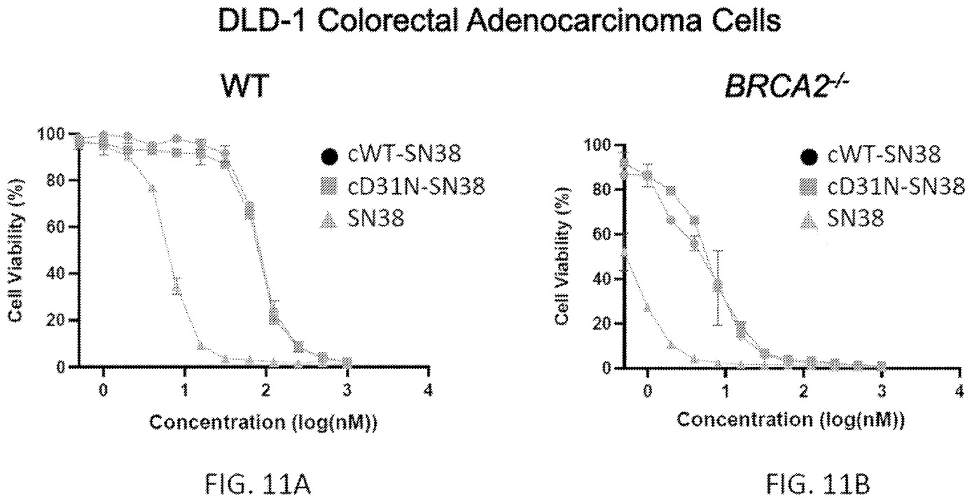

The viability assay protocol is set forth in Example 3. FIG. 11A shows the viability of wild-type (WT) DLD-1 cells treated with SN38, cWT-SN38 conjugate, and cD31N-SN38 conjugate. The EC₅₀ was measured as set forth in Table 1, below.

TABLE 1
EC₅₀ in WT DLD-1 cells.

| Treatment | EC₅₀ (nM) |
|---|---|
| WT-SN38 | 82.16 |
| D31N-SN38 | 79.68 |
| SN38 | 6.26 |

FIG. 11B shows the viability of BRCA2^{-/-} DLD-1 cells treated with SN38, cWT-SN38 conjugate, or cD31N-SN38 conjugate. The EC₅₀ was measured as set forth in Table 2, below.

TABLE 2
EC₅₀ in BRCA^{-/-} DLD-1cells.

| Treatment | EC₅₀ (nM) |
|---|---|
| WT-SN38 | 5.27 |
| D31N-SN38 | 6.25 |
| SN38 | 0.65 |

Treatment with SN38 or either 3E10 conjugate reduced cell viability, with the highest levels of cell death seen in the non-conjugated SN38 group. These findings were consistent with the Western blot results showing that treatment with SN38 induced the highest levels of DNA damage. This example demonstrates that 3E10-SN38 conjugates can effectively kill colorectal cancer cells by inducing DNA damage via delivery of SN38. Free SN38 is more potent than 3E10-SN38 conjugates, as expected, but would also be more toxic in vivo. This data shows that both WT- and D31N-3E10 ADCs are active.

### Example 6: CD31N-SN38 Conjugates Reduce Tumor Growth in a Mouse Model of Melanoma

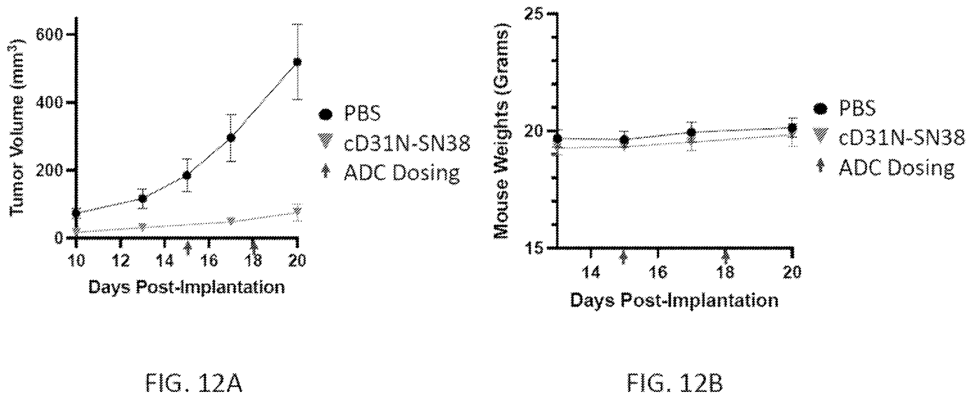

A pilot study was conducted to investigate the safety and potency of the cD31N-SN38conjugate in vivo using a melanoma mouse model. C57BL/6 mice were subcutaneously implanted with B16 melanoma cancer cells and were treated with either cD31N-SN38 conjugate (n=2 mice) or phosphate buffered saline (PBS) control (n=9 mice) at day 15 and 18. Tumor volume was measured over time. As shown in FIG. 12A, tumor growth was reduced after treatment with 12.5 mg/kg of the cD31N-SN38 conjugate compared to PBS. Mouse weight was also monitored during the study period to identify detrimental effects of the treatment that would suggest non-specific toxicity to healthy tissue. FIG. 12B shows no changes in the weight of the mice treated with the cD31N-SN38 conjugate, as compared to the mice treated with PBS.

This example demonstrates that administration of the cD31N-SN38 conjugate reduces tumor growth in mice without evidence of non-specific toxicity.

### Example 7: Assessing the Long-Term Effects of a Single Dose of SN38 or c3E10-SN38 Conjugates on DLD-1 Cancer Cell Growth

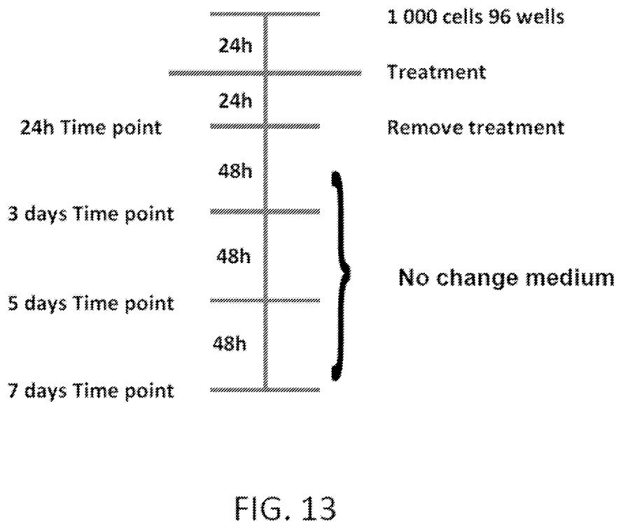

The effects of treatment with the SN38 or 3E10-SN38 conjugates on the colorectal cancer DLD-1 cell line were investigated. WT and BRCA2^{-/-} deficient DLD-1 cells were tested. FIG. 13 provides the study protocol and timeline. Each well of a 96-well plate was loaded with 1000 DLD-1 cells. 24 hours post-plating, cells were treated with different concentrations of cWT-SN38 conjugate, cD31N-SN38 conjugate, or SN38 for 24 hours. After treatment, the media was replaced with fresh treatment-free media and was not changed again. Cell viability and half maximal effective concentration (EC₅₀) were tracked every 48 hours post-treatment on days 3, 5, and 7. Half maximal effective concentration (EC₅₀) is a measure of the concentration of a drug, antibody or toxicant which induces a response halfway between the baseline and maximum after a specified exposure time.

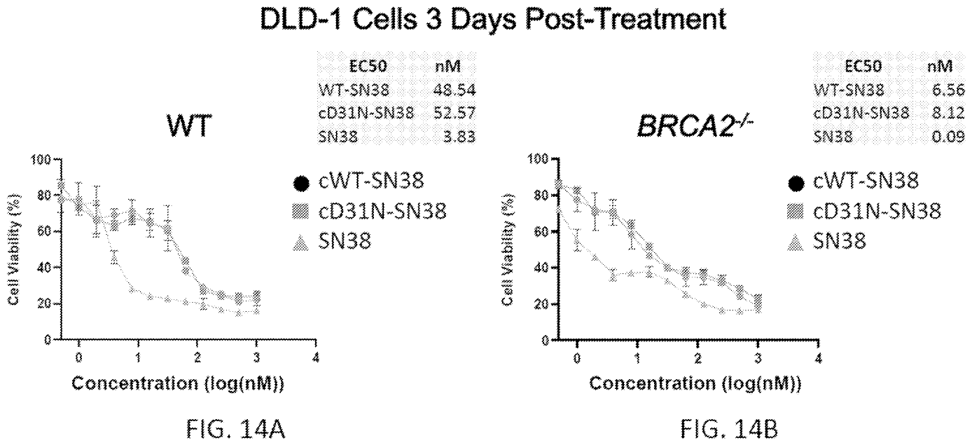

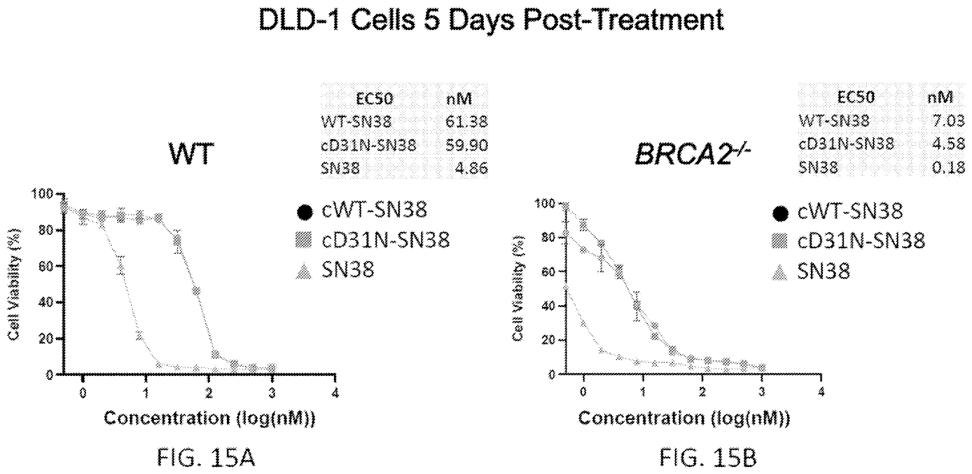

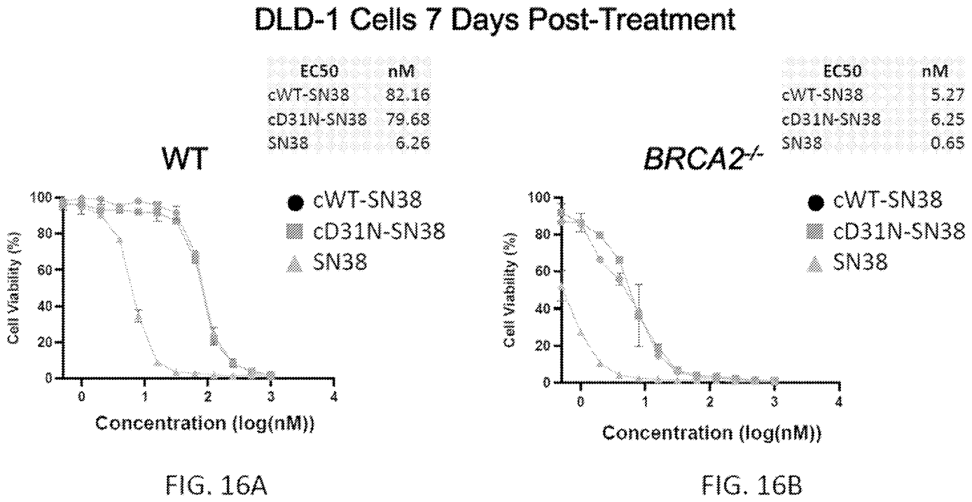

FIGS. 14A and 14B, 15A and 15B, and 16A and 16B show the results of treatment with SN38, cD31N-SN38, and wild type 3E10-SN38 conjugates (cWT-SN38) on WT and BRCA2′-DLD-1 cells on days 3, 5, and 7, respectively. Cell viability was reduced in a dose-dependent manner in each treatment group. At the highest concentrations all treatment groups reduced cell viability to about 0% 7 days post-treatment. At each time point tested, the EC₅₀ values of cWT-SN38 conjugate and cD31N-SN38 conjugate were comparable. However, non-conjugated SN38 had a lower EC₅₀ value than both 3E10-SN38 conjugates.

This Example demonstrates that a single dose of SN38 or 3E10-SN38 conjugates can have long-term effects on cancer cell growth and that these effects are dose dependent.

### Example 8: Increasing Duration of SN38 or cD31N-SN38 Conjugate Treatment Enhances Cytotoxicity

To determine whether the EC₅₀ values would change based on the duration of the treatment with 3E10-SN38 conjugates, WT or BRCA2^{-/-} DLD-1 cells were treated with different concentrations of SN38, cD31N antibody, or cD31N-SN38 conjugate and cell viability, and the EC₅₀ values were tracked over time. Shorter term treatment was defined as a 24 hour treatment followed by a change to fresh treatment-free medium. Longer term treatment was defined as treatment without a change of medium for 7 days (e.g., the treatment is not removed).

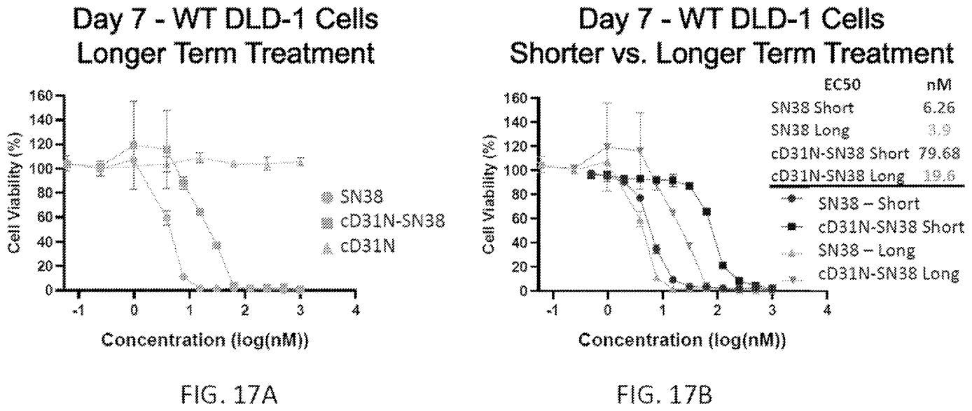

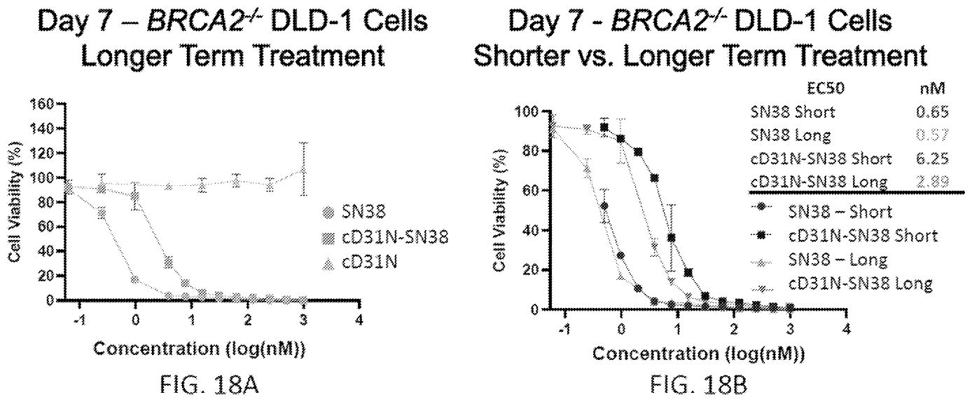

FIGS. 17 and 18 show the cell viability of of WT (FIG. 17) and BRCA2^{-/-} (FIG. 18) DLD-1 cells at day 7 of longer term treatment. DLD-1 cell viability was reduced when treated with SN38 or cD31N-SN38 conjugate (FIGS. 17A and 18A). The cD31N antibody alone had no effect on cell viability. FIGS. 17B and 18B show DLD-1 cell viability and EC₅₀ values after longer or shorter term treatment. Longer term treatment with either SN38 alone or the cD31N-SN38 conjugate resulted in lower cell viability and lower EC₅₀ values than with shorter term treatment. Both shorter and longer term treatment of SN38 resulted in lower cell viability and EC5 values than shorter or longer term treatment with cD31N-SN38 conjugates. Importantly, longer term treatment with cD31N-SN38 resulted in lower cell viability and EC₅₀ values than shorter term treatment. The results were consistent between WT and BRCA2- DLD-1 cells.

This Example demonstrates that longer term treatment (e.g., prolonged exposure to SN38 or 3E10-SN38 conjugates) increases the cytotoxicity of SN38 as measured by cell viability and EC₅₀ values.

### Example 9: SN38 and 3E10-SN38 Conjugates Reduce Survival of DLD-1 Cells as Measured by Clonogenic Assays

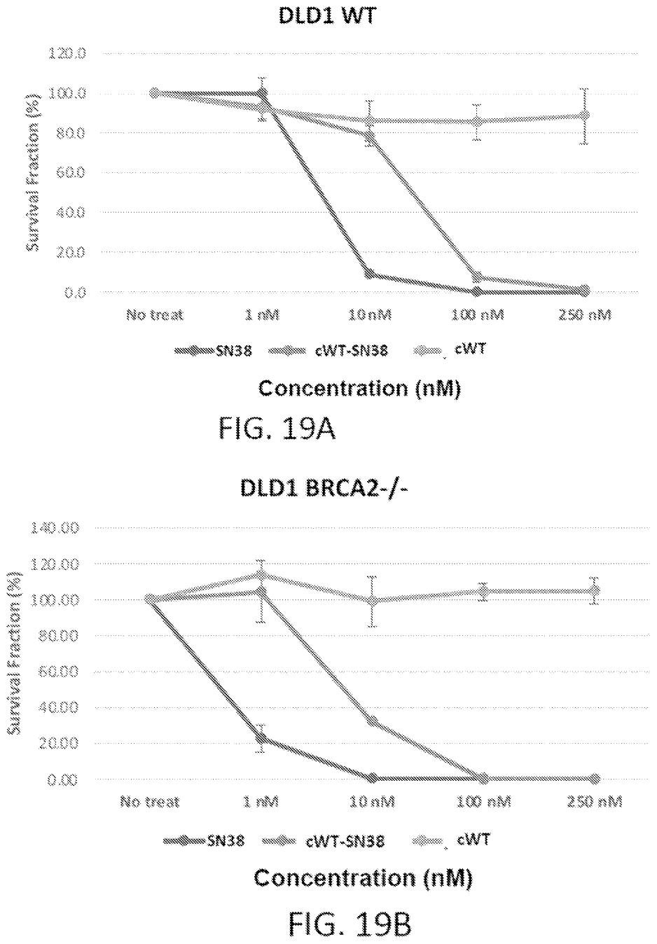

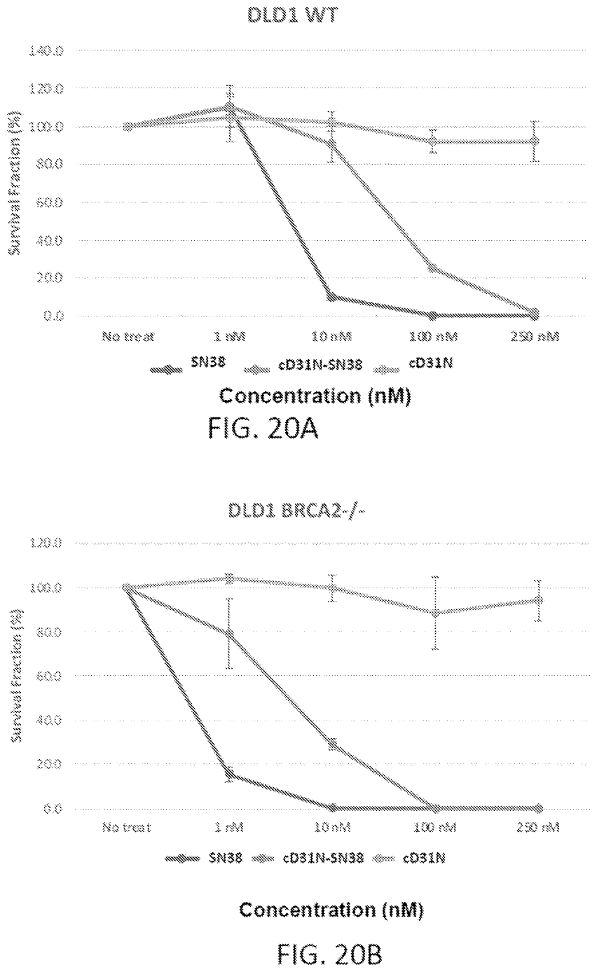

Clonogenic or colony formation assays are in vitro assays that can measure the survival or growth of cells on a single-cell level based on the ability of a single cell to grow into a colony. WT or BRCA2^{-/-} DLD-1 cells were plated with different concentrations of SN38, 3E10 antibody, or 3E10-SN38 conjugate added into the clonogenic assay media. After 7 days for WT cells or 10 days for BRCA2^{-/-} cells, colony numbers were counted to quantify DLD-1 cell survival. FIG. 19 shows the results of a clonogenic assay comparing the effects of SN38, 3E10-SN38 conjugate, and 3E10 antibody on WT (FIG. 19A) and BRCA2^{-/-} (FIG. 19B) DLD-1 cells. Cells treated with SN38 or 3E10-SN38 conjugate had reduced cell survival, while cells treated with the 3E10 antibody alone showed no change in cell survival. Consistent with earlier results, DLD-1 cell survival was concentration-dependent. The same assays were carried out with cD31N antibody and cD31N-SN38 conjugate and the results are shown in FIG. 20. As shown in FIGS. 20A and 20B, results were consistent with cWT 3E10 antibody and conjugate.

This Example further demonstrates that treatment with the 3E10 antibody does not affect cell survival, and that treatment with SN38 or 3E10-SN38 conjugates reduces cell survival in a concentration-dependent manner.

### Example 10: SN38 and 3E10-SN38 Conjugates Induce DNA Damage in DLD-1 Cells

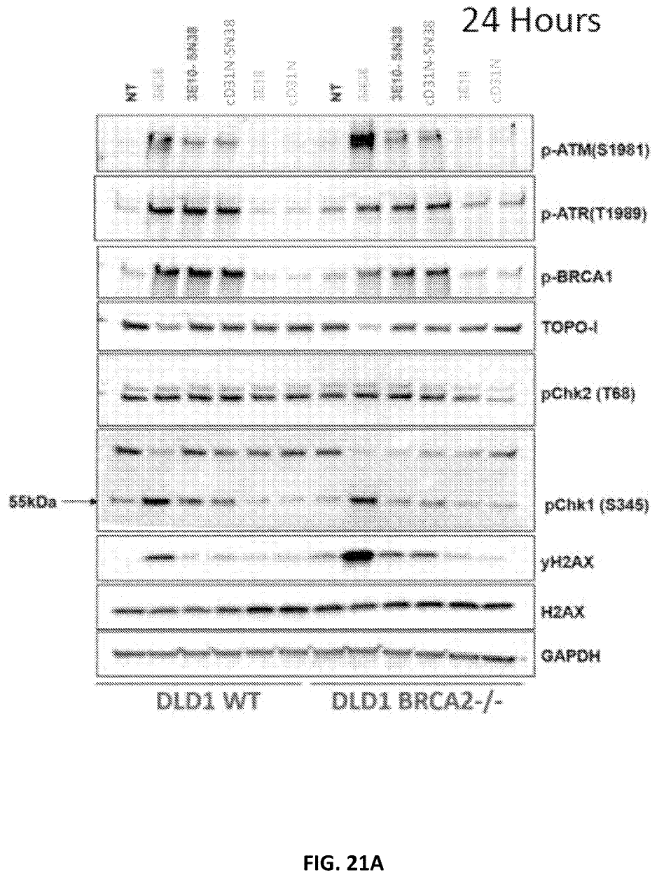

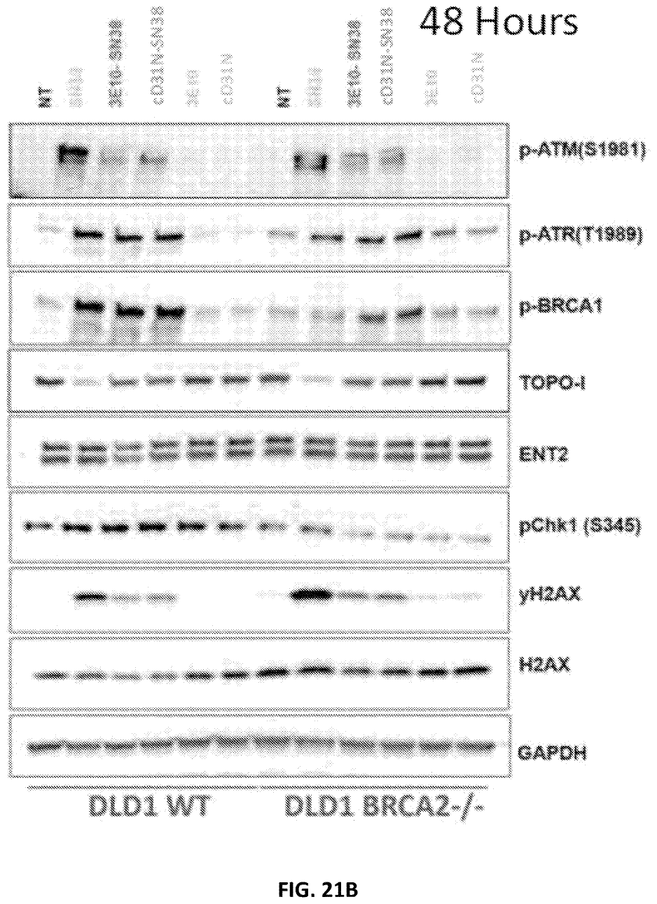

DNA damage caused by SN38 or 3E10-SN38 conjugates in WT and BRCA2^{-/-} DLD-1 cells was assessed by measuring the levels of proteins involved in DNA damage response, p-ATM, p-ATR, pBRCA1, Topoisomerase 1 (TOPO-1), pChk2, pChk1, γH2AX and H2AX. DLD-1 cells were treated with 100 nM of SN38, cWT antibody, cD31N antibody, cWT-SN38 conjugate, or cD31N-SN38 conjugate and collected for Western blot after 24 (FIG. 21A) and 48 hours (FIG. 21B). No treatment (NT) was used as control.

The 24 hour results are shown in FIG. 21A. WT and BRCA2^{-/-} DLD-1 cells were treated with 100 nM of SN38, 3E10-SN38, cD31N-SN38 conjugate, 3E10 antibody, or cD31N antibody and the Western blot results were analyzed for evidence of specific protein markers of DNA damage. Proteins of the most interest were γH2AX and phosphorylated-ATM (p-ATM), p-ATR, and p-CHK1, biomarkers for DNA double strand breaks and DNA damage. FIG. 21A is a western blot of WT DLD-1 and BRCA2^{-/-} DLD-1 cells 24 hours after treatment with SN38, 3E10-SN38 conjugate, 3E10-cD31N-SN38 conjugate, the wild-type 3E10 antibody, and the 3E10-cD31N antibody. As shown, 7H2AX was induced in WTDLD-1 and BRCA2^{-/-} DLD-1 cells treated for 24 hours with SN38, 3E10-SN38 conjugate, cD31N-SN38 conjugate, 3E10 antibody, and cD31N antibody. The γH2AX protein was not detected in the control cells.

FIG. 21B is a Western blot of WT DLD-1 and BRCA2^{-/-} DLD-1 cells after 48 hours of treatment with SN38, 3E10-SN38 conjugate, 3E10-cD31N-SN38 conjugate, 3E10 antibody, and 3E10-cD31N antibody. As shown, 7H2AX was detected in WT DLD-1 after 48 hours of treatment with SN38, 3E10-SN38 conjugate, and 3E10-D31N-SN38 conjugate, but was not detected in WT DLD-1 cells treated with 3E10 antibody, and cD31N antibody. Non-treated control cells did not show induction of γH2AX in WT or BRCA2i- DLD-1 cells. At both 24 and 48 hour time points, treatment with SN38, 3E10-SN38 conjugate, or cD31N-SN38 conjugate increased the protein levels of DNA damage response proteins such as γH2AX, p-ATM, and p-ATR. Treatment with SN38, 3E10-SN38 conjugate, or cD31N-SN38 conjugate also reduced TOPO-1 levels. Importantly, DNA damage response proteins, such as γH2AX were not detected, or only detected at very low levels in the NT, cWT 3E10 antibody and cD31N antibody groups.

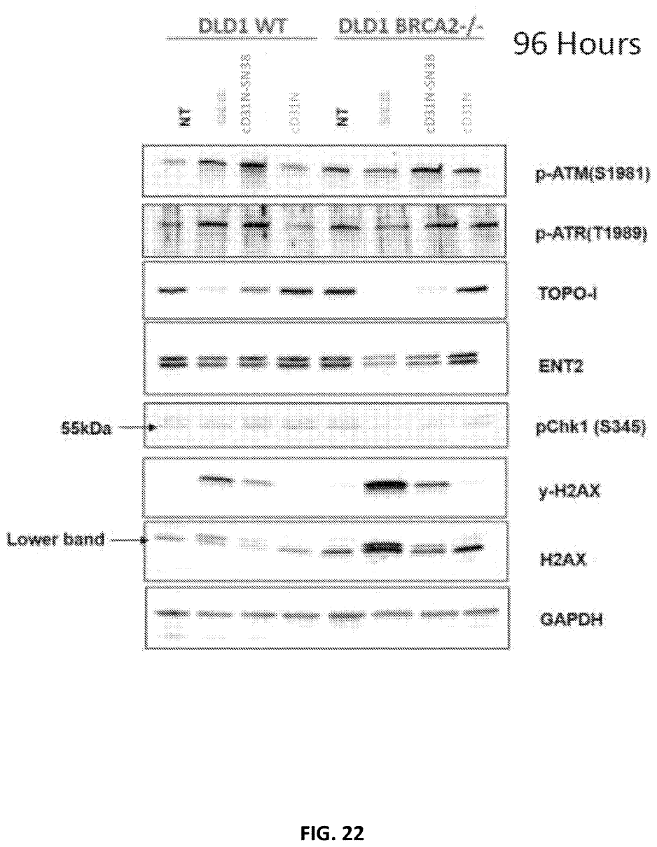

To measure the expression of DNA damage response proteins at a later time point, DLD-1 cells were treated with 100 nM of SN38, cD31N antibody, or cD31N-SN38 conjugate and collected for Western blot after 96 hours (4 days). Results at the 96 hour time point were consistent with the 24 and 48 hour results. As shown in FIG. 22, DLD-1 cells treated with SN38 or cD31-SN38 conjugate had increased expression of DNA damage response proteins, such as 7H2AX, p-ATM, and p-ATR. TOPO-1 levels were also reduced in SN38 and cD31-SN38 treated DLD-1 cells.

This Example further demonstrates a SN38 mediated mechanism of action, rather than the 3E10 antibody in the 3E10-SN38 conjugates.

### Example 11: Treatment of HCT116 Cells with the SN38 or cD31N-SN38 Conjugate Causes Cell Death

The effects of treatment with SN38 and 3E10-SN38 conjugates on HCT116 colorectal carcinoma cells were investigated.

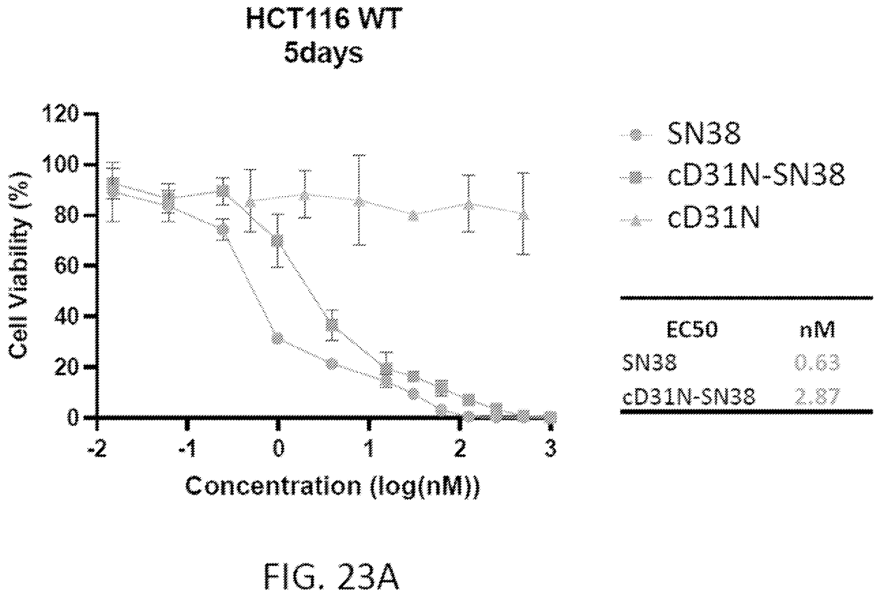

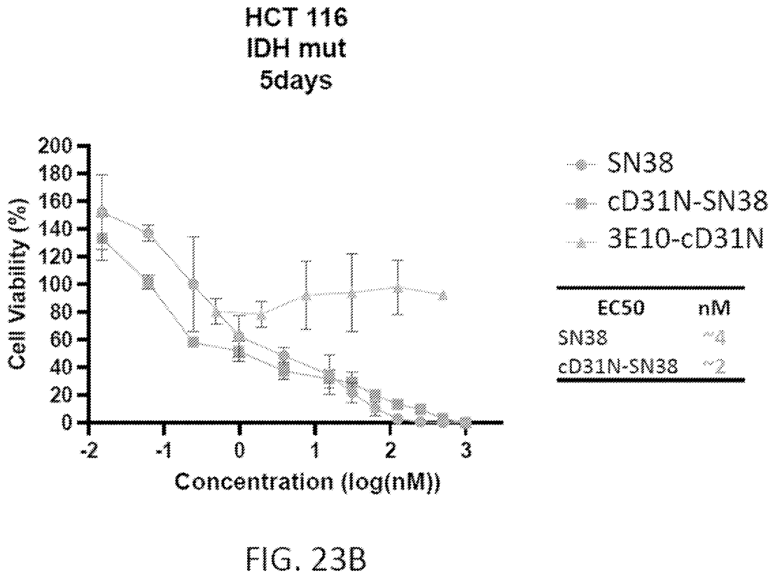

WT and mutant IDH1 (IDH1^{mut}) HCT116 cells were treated with different concentrations of SN38, cD31N antibody, or cD31N-SN38 conjugate. The effects of 3E10-cD31N-SN38 conjugate treatment on HCT116 WT and HCT116IDH1^{mut} were investigated. FIG. 23A shows the cell viability and the EC₅₀ value of HCT116 colorectal cancer cells treated with SN38, 3E10-cD31N-SN38 conjugate, and 3E10-cD31N antibody for 5 days. Cells treated with the cD31N antibody alone showed little change in cell viability after 5 days of treatment. Cells treated with SN38 and the xD31N-SN38 conjugate resulted in substantial loss in cell viability after 5 days of treatment, which is indicative of a SN38 mediated mechanism of action.

FIG. 23B shows the cell viability and the EC₅₀ value of HCT116- IDH1^{mut} cells treated with SN38, 3E10-cD31N-SN38 conjugate, and 3E10-cD31N antibody for 5 days. Cells treated with the cD31N antibody alone showed little change in cell viability after 5 days of treatment. Cells treated with SN38 and the cD31N-SN38 conjugate resulted in substantial loss in cell viability after 5 days of treatment which is indicative of a SN38 mediated mechanism of action.

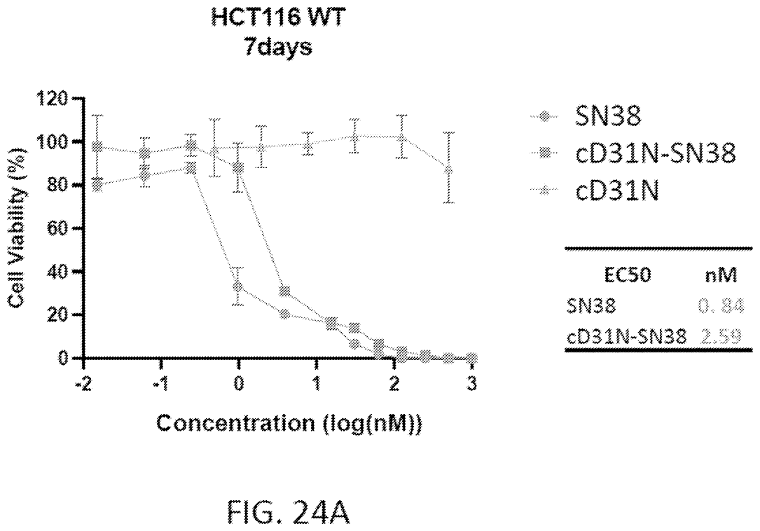

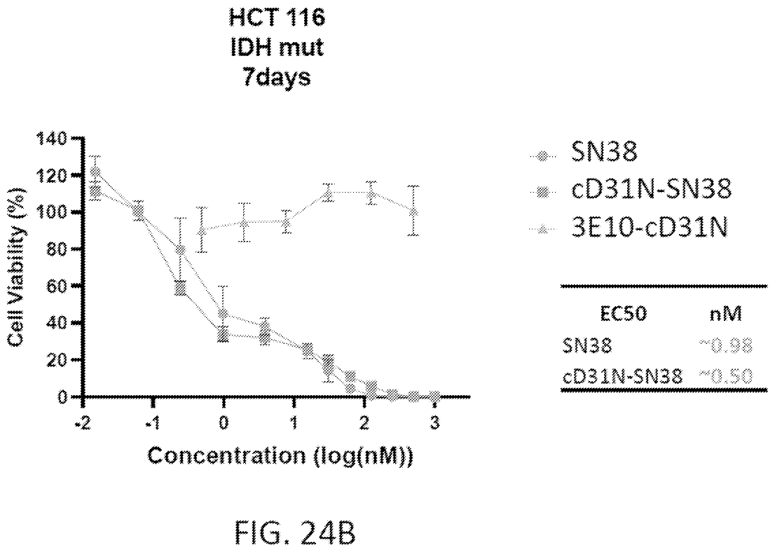

FIG. 24A shows the cell viability and the EC5 value of colorectal cancer HCT116 cells treated with SN38, cD31N-SN38 conjugate, and cD31N antibody for 7 days. Cells treated with the cD31N antibody alone showed little change in cell viability after 7 days of treatment. Cells treated with SN38 and cD31N-SN38 conjugate resulted in substantial loss in cell viability after 7 days of treatment, which is indicative of a SN38 mediated mechanism of action.

FIG. 24B shows the cell viability and the EC₅₀ value of HCT116- IDH1^{mut} cells treated with SN38, cD31N-SN38 conjugate, and cD31N antibody after 7 days of treatment. Cells treated with cD31N antibody alone showed little change in cell viability after 7 days of treatment. Cells treated with SN38 and cD31N-SN38 conjugate resulted in substantial loss in cell viability 7 days post treatment, which is indicative of a SN38 mediated mechanism of action.

This example and the resulting EC₅₀ values shown in FIGS. 24A-B demonstrate that cell killing is likely due to a SN38 mediated mechanism of action, and that the 3E10-cD31N antibody is not inducing a substantial amount of DNA damage on its own.

These results are consistent with results in other cancer cells and further demonstrates a SN38 mediated mechanism of action, rather than the 3E10 antibody in the 3E10-SN38 conjugates. Example 12: SN38 and 3E10-SN38 Conjugates Reduce Survival of HCT116 Cells as Measured by Clonogenic Assays.

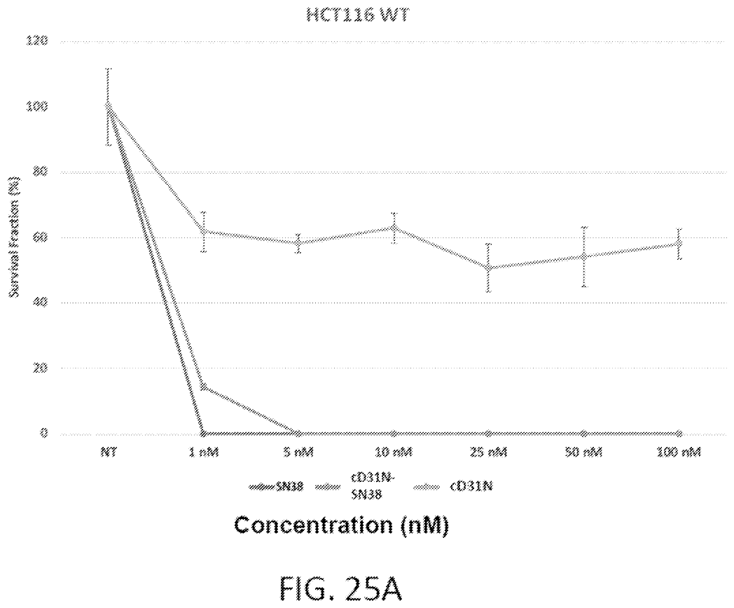

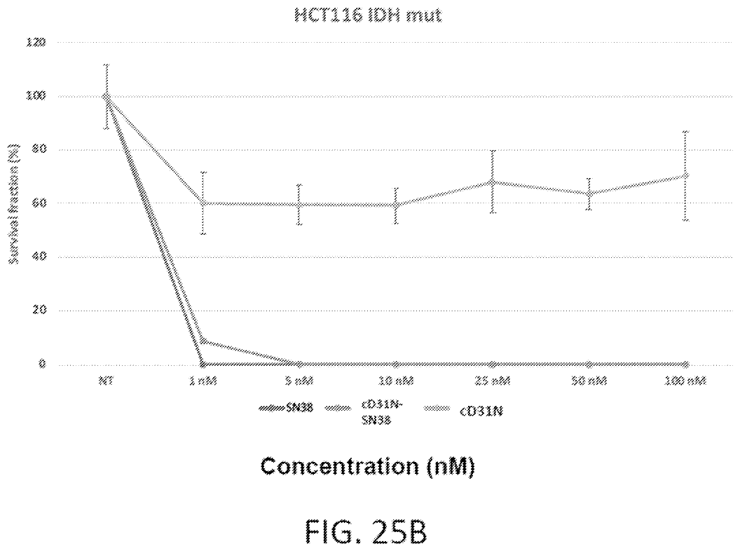

The effects of treatment with SN38 or 3E10-SN38 conjugates on HCT116 cells were tested in clonogenic assays as previously described. WT and IDH1^{mut} HCT116 cells were treated with SN38, cD31N antibody, or cD31N-SN38 conjugate. Cell survival of WT (FIG. 25A) and IDH1^{mut} HCT116 cells (FIG. 25B) was quantified after 7 days for WT cells or 10 days for BRCA2^{-/-} cells. Cells treated with SN38 and the 3E10-cD31N-SN38 conjugate had low levels of cell survival in a concentration-dependent manner and cells treated with the cD31Nantibody alone showed little change in cell survival. Treatment with 1 nM of SN38 resulted in 0% survival fraction of WT HCT116 cells. Treatment with 5 nM of cD31N-SN38 conjugate resulted in 0% survival fraction of WT HCT116 cells.

FIG. 25B shows the results of a clonogenic assay comparing the effects of SN38, the cD31N-SN38 conjugate, and the cD31N antibody on HCT116-IDH1^{mut} cells. Cells treated with SN38 and the 3E10-cD31N-SN38 conjugate had low levels of cell survival. Cells treated with the cD31N antibody alone showed little change in cell survival.

This example demonstrates again that with the cD31N antibody alone does not affect cell survival, but that conjugating the cD31N antibody with SN38 does affect cell survival. Example 13: SN38 and Humanized 3E10-SN38 Conjugates Induce DNA Damage in HCT116 Cells.

Additional 3E10 ADCs with SN38 were synthesized as described above by conjugating SN38 with a 3E10-D31N humanized antibody (V66-SN38).

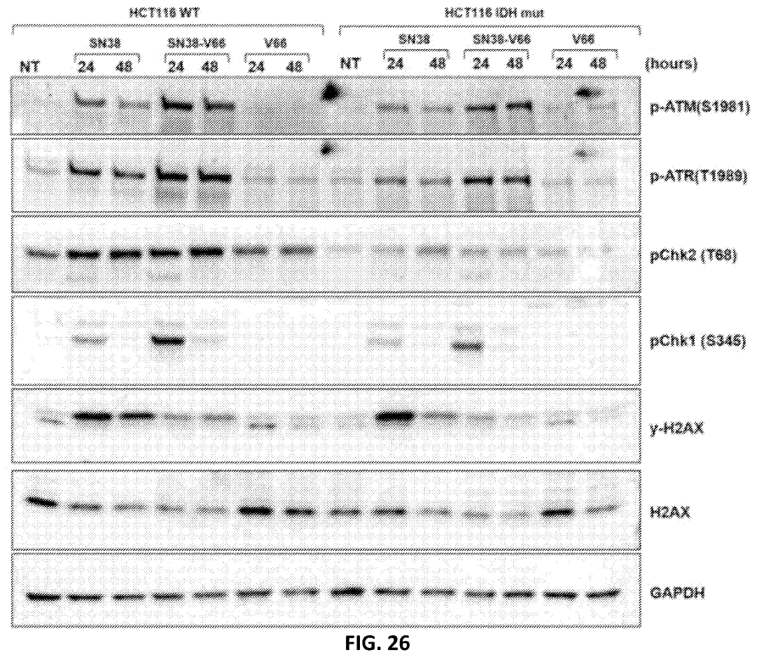

DNA damage induced by SN38 or the humanized 3E10-SN38 conjugates in WT and IDH1^{mut} HCT116 cells was assessed by measuring the levels of proteins involved in DNA damage response, p-ATM, p-ATR, pChk2, pChk1, 7H2AX and H2AX. HCT116 cells were treated with 100 nM of SN38, V66 antibody, or V66-SN38 conjugate and collected for Western blot after 24 hours and the results are shown in FIG. 26. No treatment (NT) was used as control. Treatment with SN38, V66 antibody, or V66-SN38 conjugate increased the protein levels of DNA damage response proteins such as γH2AX, p-ATM, and p-ATR. Importantly, DNA damage response proteins, such as γH2AX were not detected, or only detected at very low levels in the NT and V66 antibody groups.

This Example further demonstrates a SN38 mediated mechanism of action, rather than the 3E10 antibody in the 3E10-SN38 conjugates. This Example also indicates that humanized 3E10-drug conjugates work as effectively as chimeric 3E10-drug conjugates.

### Example 14: Cell Viability of LoVo Cells after ADC Treatment for 7 Days

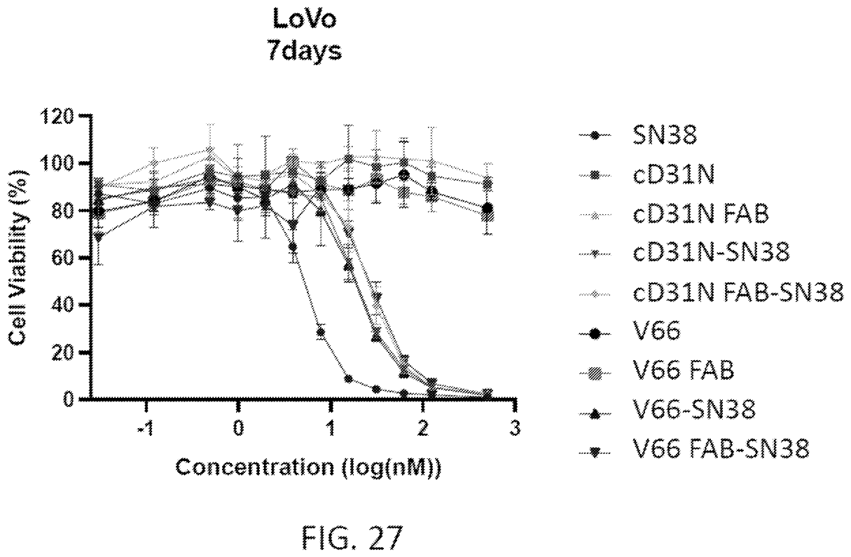

LoVo colorectal cancer cells were treated with SN38, cD31N antibodies, V66 3E10 antibodies, and conjugates of the present disclosure. FIG. 27 shows the cell viability of colorectal cancer LoVo cells after 7 days of treatment with SN38, cD31N antibody, cDN31 Fab antibody, cD31N-SN38 conjugate, cDN31-Fab-SN38 conjugate, V66 antibody, V66-Fab antibody, V66-SN38 conjugate, and V66-Fab-SN38 conjugate. While SN38 had the strongest effect on cell viability, LoVo cells treated with SN38 conjugated to any 3E10 antibodies reduced cell viability in a concentration-dependent manner and at the highest concentrations cell viability was reduced to about 0%. No significant change in cell viability was observed in cells treated with any of the non-conjugated 3E10 antibodies (the cD31N Fab antibody, the D31N antibody, the V66 antibody, or the V66 Fab antibody). This suggests a common SN38 mediated mechanism of action. The EC₅₀ values were calculated and are set forth below in Table 3.

TABLE 3
EC₅₀ values in LoVo Cells

| Treatment | EC₅₀ (nM) |
|---|---|
| SN38 | 5.8 |
| cD31N-SN38 | 20.9 |
| cD31N Fab-SN38 | 25.9 |
| V66-SN38 | 20.1 |
| V66 Fab-SN38 | 32.6 |
| cD31N Fab-SN38 | 11.3 |
| (Normalized to SN38) | |
| V66 Fab-SN38 | 14.2 |
| (Normalized to SN38) | |

LoVo cells were effectively killed by the cD31N Fab-SN38 conjugate, the V66 Fab-SN38 conjugate, the V66-SN38 conjugate, and the cD31N-SN38. Therefore, SN38 can be conjugated with humanized antibodies or antibody fragments and be highly effective in reducing cell viability.

### Example 15: Treatment of SKOV3 Cells with the SN38 or 3E10-SN38 Conjugate Causes Cell Death

The effect of SN38 and 3E10-SN38 conjugates were investigated in SKOV3 ovarian cancer cells. SKOV3 cells were treated with SN38, various 3E10 antibodies and 3E10-SN38 conjugates.

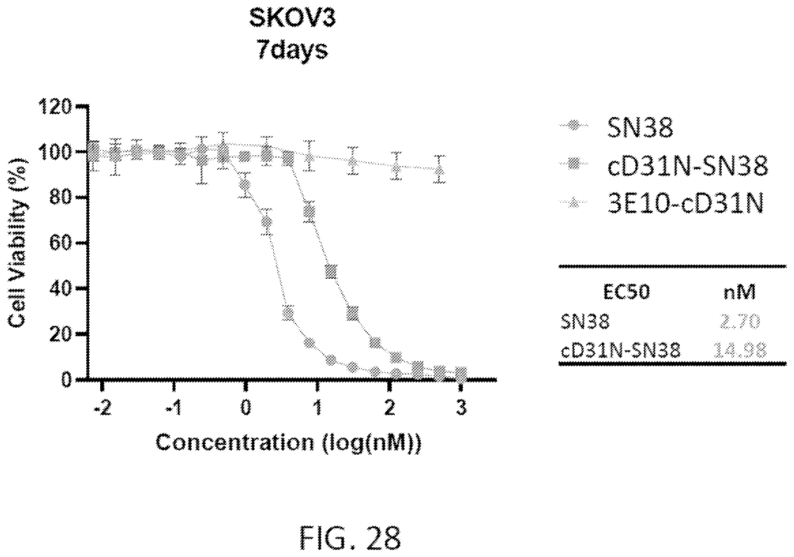

FIG. 28 shows the cell viability of SKOV3 cells at 7 days of treatment with different concentrations of SN38, cD31N antibody, or cD31N-SN38 conjugate. EC₅₀ values are also included.

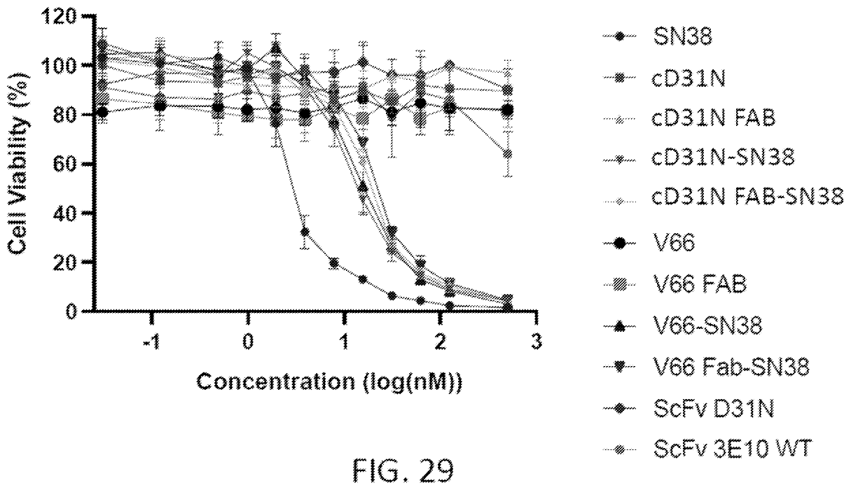

FIG. 29 shows the cell viability of colorectal cancer LoVo cells after 7 days of treatment with SN38, cD31N antibody, cDN31 Fab antibody, cD31N-SN38 conjugate, cDN31-Fab-SN38 conjugate, V66 antibody, V66-Fab antibody, V66-SN38 conjugate, and V66-Fab-SN38 conjugate. While SN38 had the strongest effect on cell viability, LoVo cells treated with SN38 conjugated to any of the 3E10 antibodies reduced cell viability in a concentration-dependent manner and at the highest concentrations cell viability was reduced to about 0%.

In all experiments in SKOV3 cells, both SN38 and 3E10-SN38 conjugates reduced cell viability in a concentration dependent manner and at the highest concentrations cell viability was reduced to about 0%. No significant change in cell viability was observed in cells treated with any of the non-conjugated 3E10 antibodies. The EC₅₀ values were calculated and are set forth below in Table 4.

TABLE 4
EC₅₀ values in SKOV3 cells

| Treatment | EC₅₀ (nM) |
|---|---|
| SN38 | 2.8 |
| cD31N-SN38 | 12.7 |
| cD3IN Fab-SN38 | 17.7 |
| V66-SN38 | 14.8 |
| V66 Fab-SN38 | 20.8 |

This example demonstrates SN38 can be conjugated with humanized antibodies or antibody fragments and be highly effective in reducing cell viability. This Example also further demonstrates a SN38 mediated mechanism of action.

### Example 16: Treatment with SN38 or cD31N-SN38 Conjugate Induces DNA Damage In SKOV3 Cells

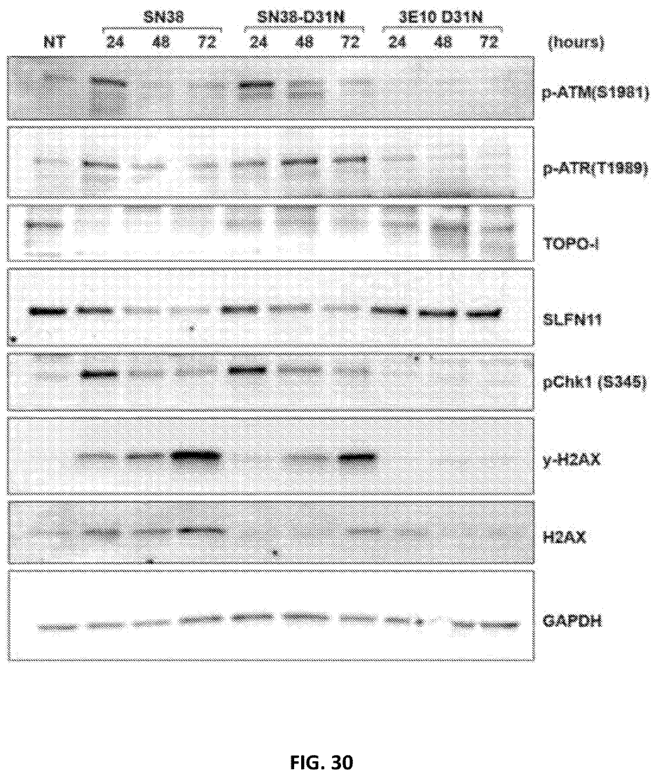

SKOV3 ovarian cancer cells were treated with 100 nM of SN38, cD31N antibody, or cD31N-SN38 as indicated and collected at 24, 48, and 72 hours of treatment for Western blot analysis to measure specific protein markers of DNA damage response as previously described. FIG. 30 shows the protein level of p-ATM, pATR, TOPO-1, SLFN11, pCHK1, γH2AX, and p-ATR as measured by Western blot. In SKOV3 cells treated with SN38 or cD31N-SN38, protein expression of DNA damage response proteins was increased. In particular, 7H2AX levels increased over time. In contrast, cells treated the non-conjugated cD31N antibody, did not exhibit increased levels of DNA damage response proteins.

This Example demonstrates that SN38 and 3E10-SN38 conjugate can effectively induce DNA damage in ovarian cancer cells. This Example also further demonstrates that DNA damage is caused by SN38, not the 3E10 antibody itself.

### Example 17: Longer Term Treatment of PEO-4 and PEO-1 Cells with SN38 or cD31N-SN38 Reduces Cell Viability

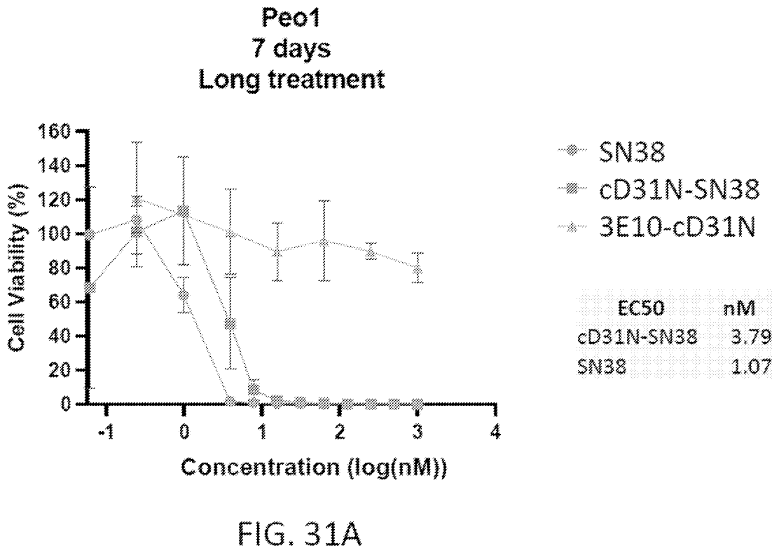

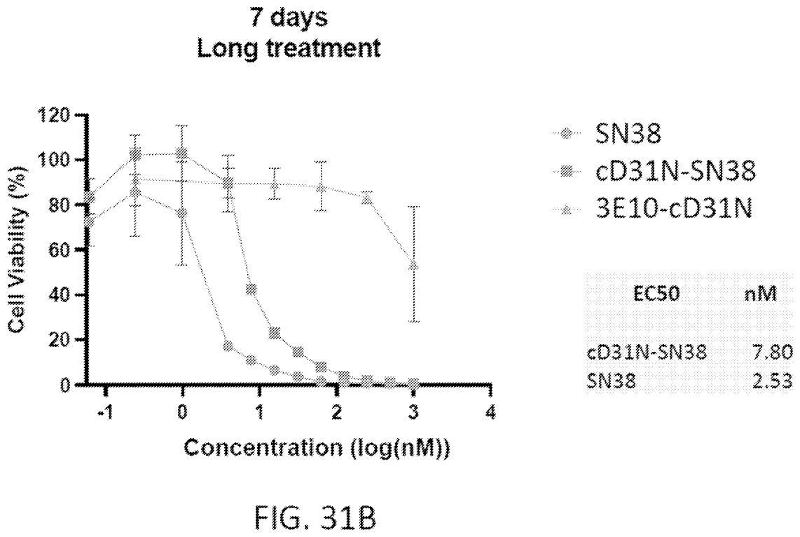

The effects of longer-term treatment with SN38 and cD31N-SN38 on PEO-4 (WT) and PEO-1 (BRCA2^{-/-}) ovarian cancer cells were investigated. FIG. 31 shows the cell viability at 7 days in PEO-1 (BRCA2^{-/-}) ovarian cancer cells (FIG. 31A) and PEO-4 (WT) ovarian cancer cells (FIG. 31B). Treatment with SN38 or cD31N-SN38 conjugate reduced cell viability in a concentration-dependent manner resulting in low EC₅₀ values. Treatment with cD31N antibody alone had little effect on cell viability.

This Example demonstrates that SN38 and 3E10-SN38 conjugate can effectively kill ovarian cancer cells after longer term treatment. This Example also further demonstrates that DNA damage is caused by SN38, not the 3E10 antibody itself.

### Example 18: Treatment with SN38 or cD31N-SN38 Conjugate Induces DNA Damage In PEO-4 and PEO-1 Cells

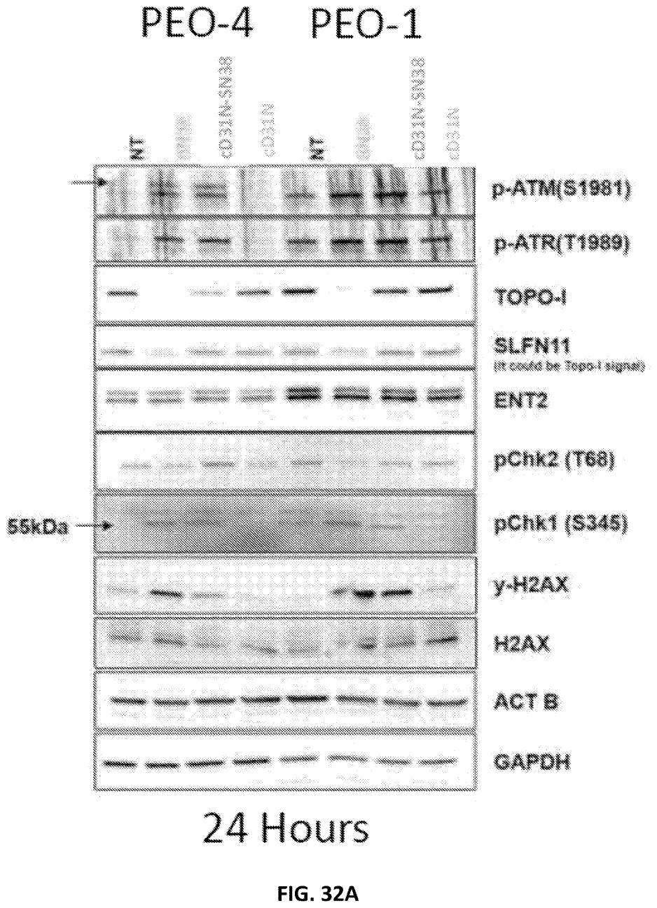

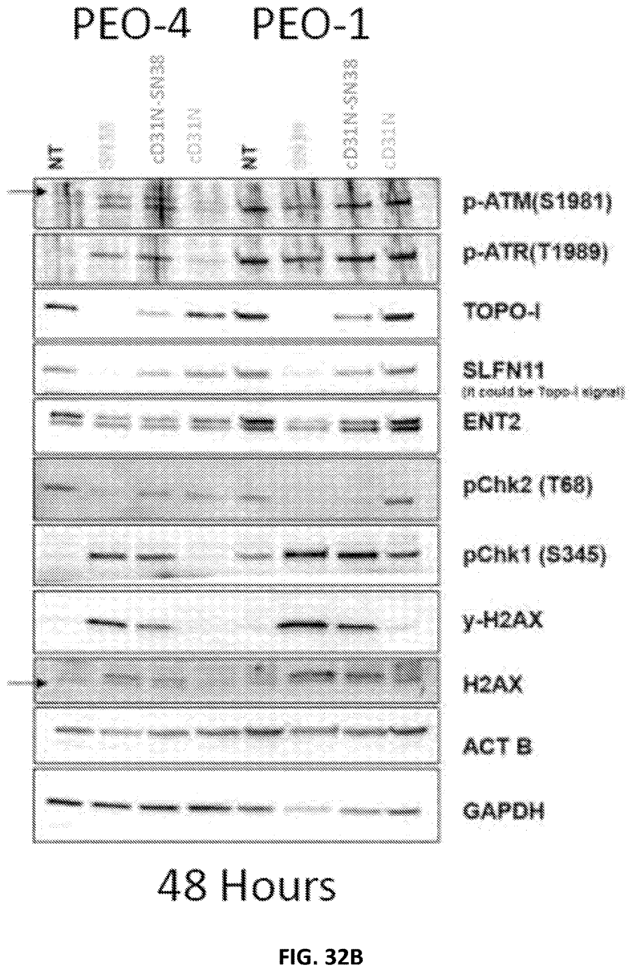

PEO-4 (WT) and PEO-1 (BRCA2^{-/-}) ovarian cancer cells were treated with 100 nM of SN38, cD31N or cD31N-SN38 and collected at 24 and 48 hours of treatment for Western blot analysis to measure specific protein markers of DNA damage response as previously described. FIG. 32 shows the protein level of p-ATM, pATR, TOPO-1, SLFN11, ENT2, pCHK1, pCHK2, 7H2AX, p-ATR, and p-CHK1, which are biomarkers for DNA double strand breaks and DNA damage as measured by Western blot after 24 hours (FIG. 32A) and 48 hours (FIG. 32B) of treatment. In PEO-4 and PEO-1 cells treated with SN38 or cD31N-SN38, protein expression of DNA damage response proteins was increased. In contrast, cells treated with the non-conjugated cD31N antibody, did not exhibit increased levels of DNA damage response proteins. This Example demonstrates that SN38 and cD31N-SN38 conjugate can effectively induce DNA damage in ovarian cancer cells. This Example also further demonstrates that DNA damage is caused by SN38, not the 3E10 antibody itself.

### Example 19: Treatment of Colonrectal Cancer with a 3E10-SN38 ADC in a Mouse Model

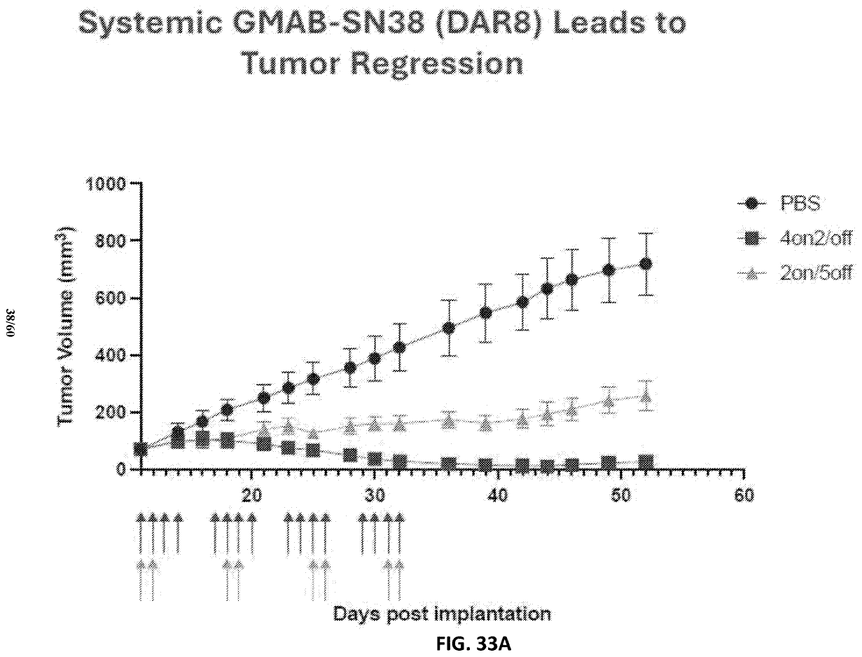

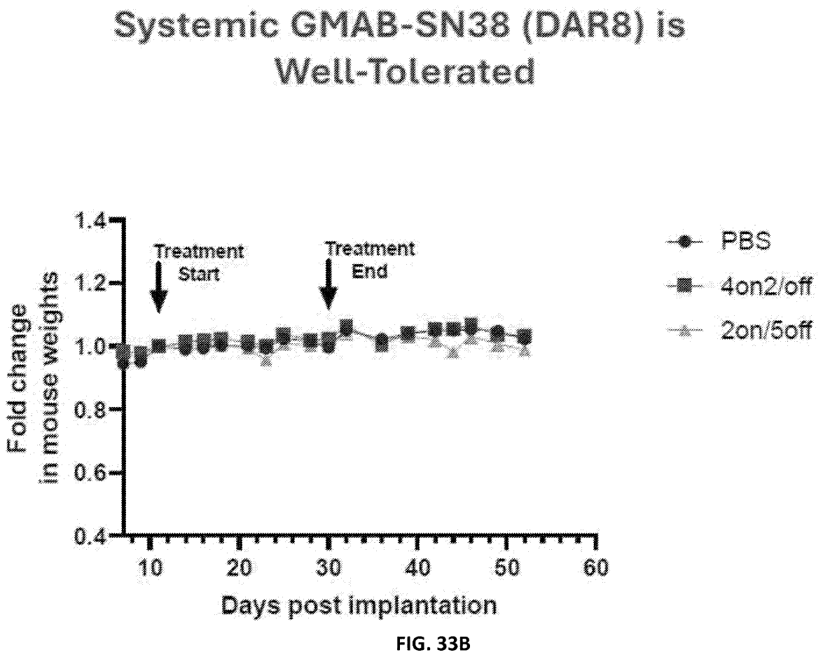

A study was conducted to investigate the safety and potency of the cD31N-SN38 conjugate in vivo using a colorectal mouse model. C57BL/6 mice were orthotopically implanted with MC38 cancer cells via intracecal injection and were treated with systemic administration of the cD31N-SN38 conjugate for four consecutive days, followed by two days without treatment, or for two consecutive days followed by five days without treatment. Tumor volume was measured over time post implantation. As shown in FIG. 33A, tumor growth was reduced after treatment of 12.5 mg/kg of the cD31N-SN38 conjugate according to both treatment protocols compared to phosphate buffered saline (PBS) control. Mouse weight was also monitored during the study period to identify detrimental effects of the treatment that would suggest non-specific toxicity to healthy tissue. FIG. 33B shows no changes in the weight of the mice treated with systemic administration of the cD31N-SN38 conjugate, as compared to the mice treated with PBS.

This example demonstrates that systemic administration of the cD31N-SN38 conjugate post implantation reduces tumor growth in mice without evidence of non-specific toxicity.

### Example 20: In Vitro Potency of a 3E10-SN38 ADC Against Different Cancer Cell Lines

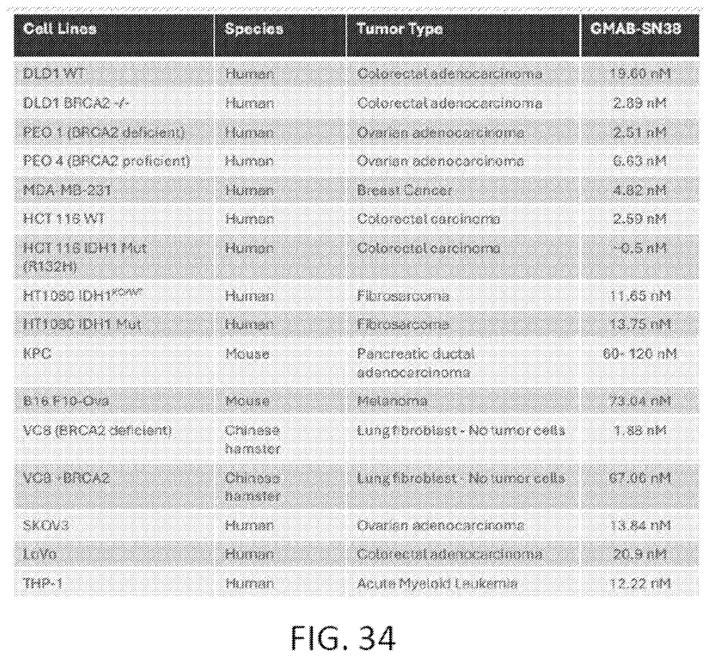

A panel of different cancer cell lines were cultured with increasing nanomolar concentrations of cD31N-SN38 conjugate and cell viability was measured. The potency of cD31N-SN38 conjugate was measured against each of the cancer cell lines shown in FIG. 34.

This example demonstrates the broad applicability and potency of the cD31N-SN38 conjugate against various cancer cell lines. The cD31N-SN38 ADC was effective in all tested cancer cell lines, regardless of genetic background or tissue of origin.

### Example 21: In Vitro Potency of a 3E10-DM1 ADC Against Different Cancer Cell Lines

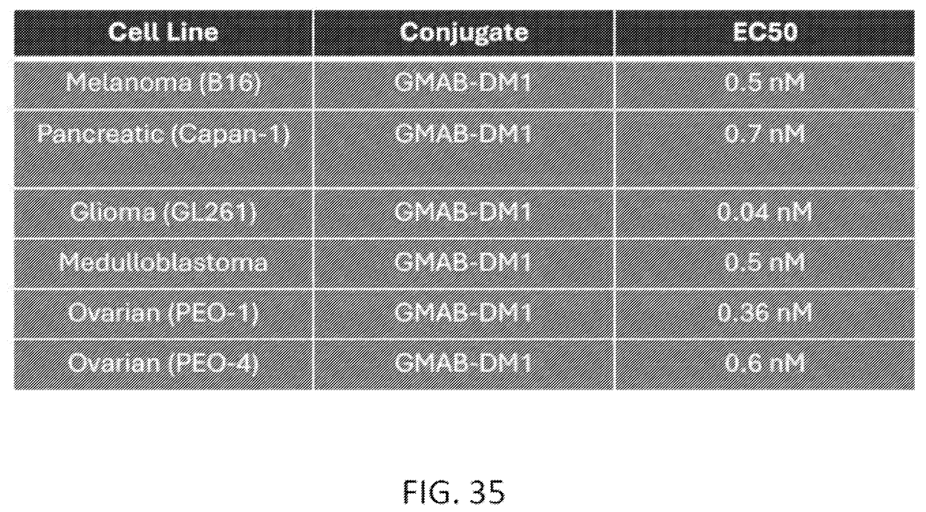

A panel of different cancer cell lines were cultured with increasing picomolar concentrations of cD31N-DM1 conjugate and cell viability was measured. The potency of cD31N-DM1 conjugate was measured against each of the cancer cell lines shown in FIG. 35.

This example demonstrates the broad applicability and potency of the cD31N-DM1 conjugate against various cancer cell lines. The cD31N-DM1 ADC was effective in all tested cancer cell lines, regardless of genetic background or tissue of origin.

### Example 22: Treatment of Cancer with a V66-SN38 (ADC) in a Colorectal Adenocarcinoma (DLD1) (BRCA2^{-/-}) Mouse Tumor Model

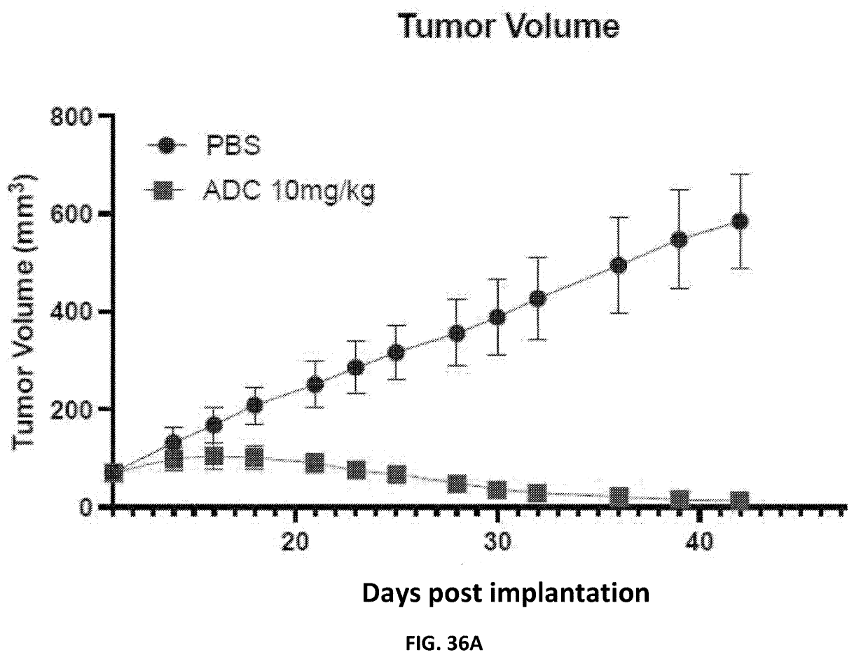

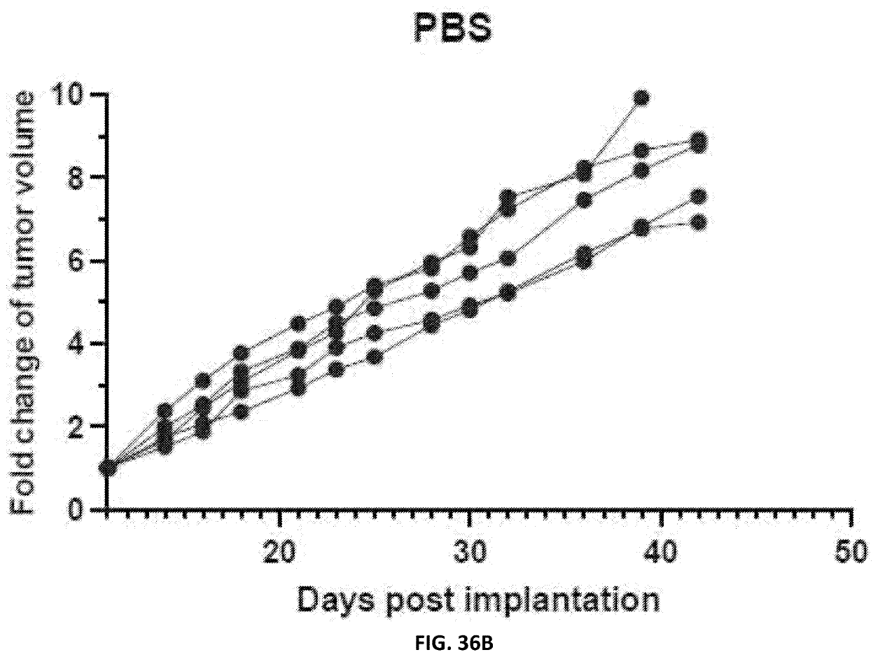

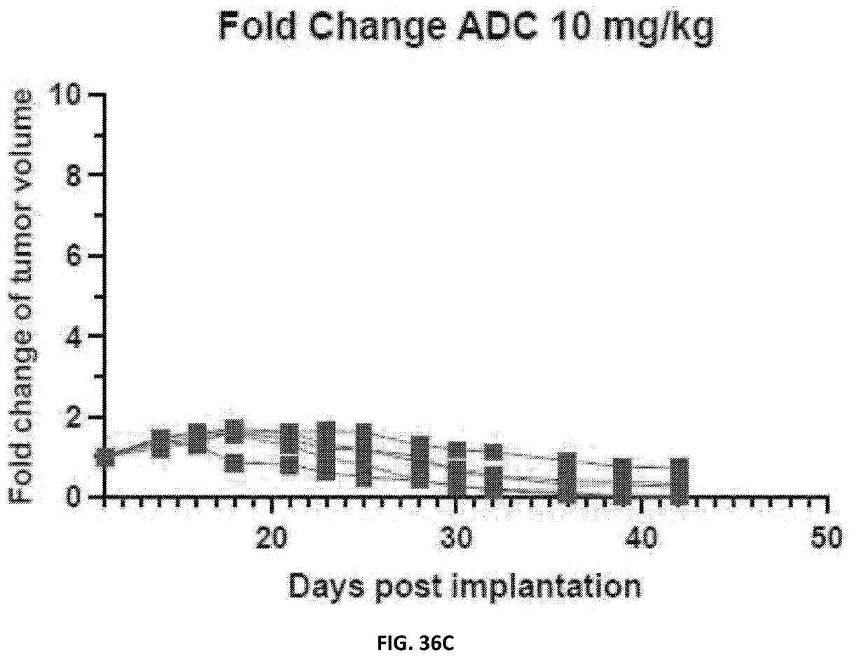

A study was conducted to investigate the potency of the V66-SN38 conjugate in vivo using a Dukes-type C colorectal adenocarcinoma (DLD1) (BRCA2^{-/-}) mouse tumor model. C57BL/6 mice with colorectal adenocarcinoma (DLD1) flank tumors were treated with either V66-SN38 conjugate (10 mg/kg; volume 100 mm3) or phosphate buffered saline (PBS) control post implantation of tumor. Tumor volume was measured over time post-implantation. As shown in FIG. 36A, tumor growth was reduced after treatment with 10 mg/kg of the V66-SN38 conjugate compared to PBS. FIG. 36B and FIG. 36C show fold change of tumor volume for V66-SN38 conjugate (10 mg/kg) or phosphate buffered saline (PBS) control.

### Example 23: In Vitro Potency of 3E10 ADCs Against a Human Dukes-Type C Colorectal adenocarcinoma (DLD1) cell line with a homozygous knock out of BRCA2

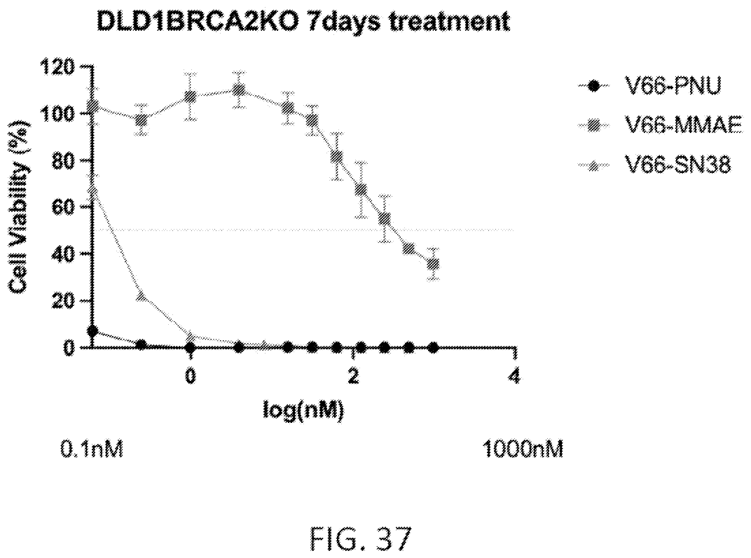

The effects of longer-term treatment with V66-PNU, V66-MMAE, and V66-SN38 conjugates against a human Dukes-type C colorectal adenocarcinoma (DLD1) cell line with a homozygous knock out of BRCA2 (BRCA2-) were investigated. FIG. 37 shows the cell viability 7 days post-treatment with V66-PNU, V66-MMAE, and V66-SN38 conjugates in a DLD1 (BRCA2′-) cancer cell line. Treatment with V66-PNU, V66-MMAE, and V66-SN38 conjugates reduced cell viability in a concentration-dependent manner resulting in low EC₅₀ values. Treatment with V66-MMAE and V66-SN38 conjugates resulted in undetectable viability of the DLD1 (BRCA2′-) cancer cell line.

This Example demonstrates that each of the V66-PNU, V66-MMAE, and V66-SN38 conjugates can effectively kill a DLD1 (BRCA2^{-/-}) cancer cell line 7 days post-treatment.

### Example 24: In Vitro Potency of 3E10 ADCs Against 3 ENT2(+) Tumor Cell Lines

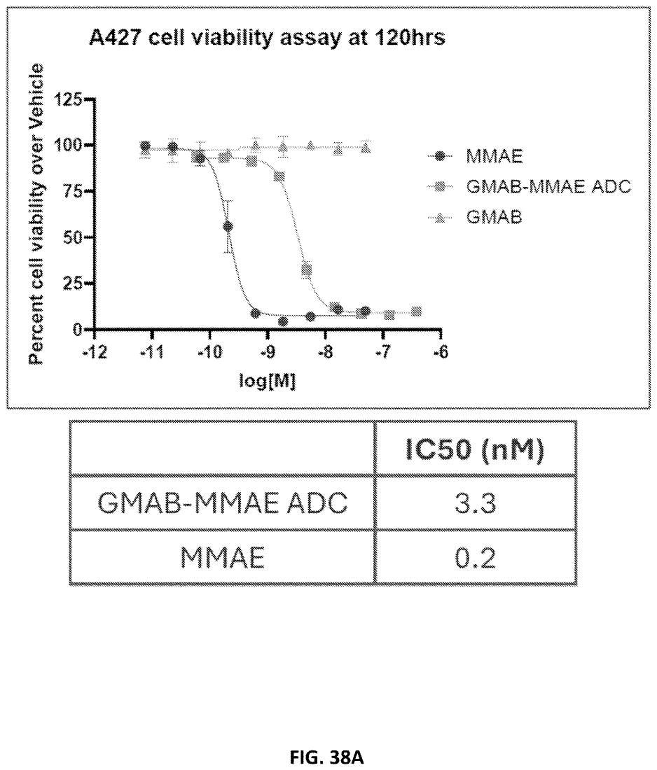

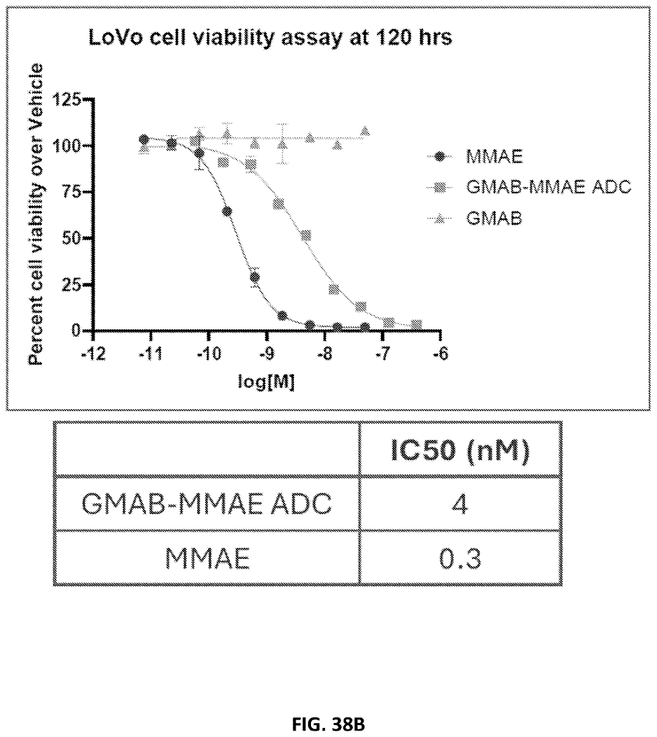

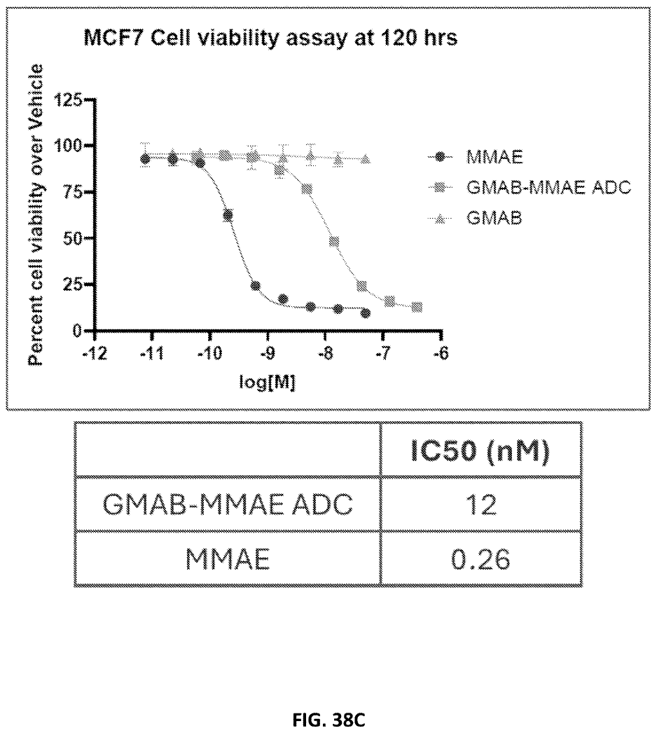

The effects of treatment with V66-MMAE conjugates and MMAE alone in 3 ENT2(+) tumor cell lines (MCF7, LOVO, A427) were investigated. FIG. 38A-38C shows the cell viability post-treatment with V66-MMAE, and V66 alone, and 3E10 in MCF7, LOVO, A427 cancer cell lines. This example demonstrates that a 3E10 ADC MMAE is exhibits potent cells killing in 3 ENT2(+) cell lines, but is 3 times more potent in A427 and LOVO compared to MCF7 possibly due to differential 3E10 uptake or differential cathepsin B expression in these cell lines.

### Example 25: Tryptic Peptide Mapping to Identify Preferential Lysine Residues for 3E10-D31N Monoclonal Antibody (V66) Conjugation

Since the variable regions and CDRs of the 3E10-D31N monoclonal antibody (V66) contain multiple lysine residues, it was investigated with conjugation in this region may have a negative impact on antibody immunoreactivity and function.

To test this hypothesis, 3E10-D31N monoclonal antibody (V66) Lys-azide conjugation intermediates were digested and mapped by mass spectrometry to characterize modified lysines in the VH and VL chains of the antibody and as shown in FIGS. 39A and 39B. As is shown in FIG. 39B, most levels of lysine modification are modest and many of the modified lysines are not implicated in antibody function. As shown in FIG. 39C, the light chain K53 is the most modified lysine (nearly⅔ instances) and is in the CDR2. These results indicate that any modification of VL K53 negatively impacted immunoreactivity of the 3E10-D31N monoclonal antibody (V66) with DNA and in turn has a negative impact on the function of an antibody-oligonucleotide conjugate (AOC).

### Example 26: Improved Cellular Internalization Of3E10-D31N Monoclonal Antibody (V66) Oligonucleotide Conjugates Utilizing Transglutaminase-Mediated Enzymatic Conjugation

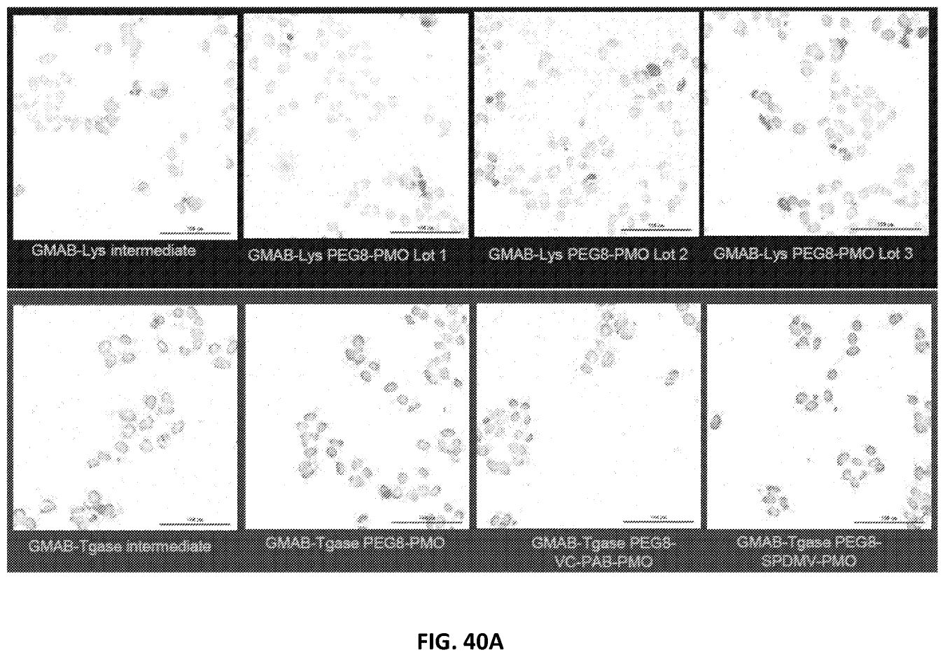

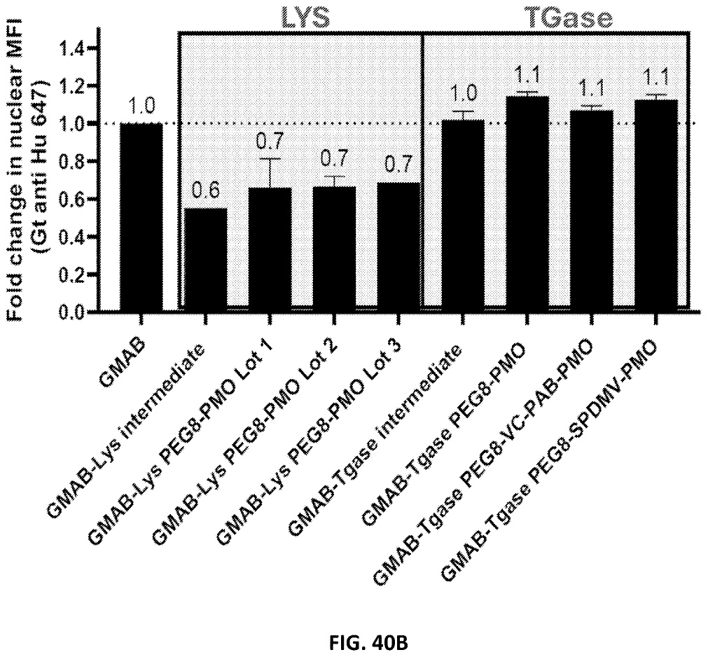

Internalization of 3E10-D31N monoclonal antibody (V66) oligonucleotide conjugates was investigated in A427 cells comparing lysine versus transglutaminase-mediated enzymatic conjugation methods. As is shown in FIG. 40A, immunohistochemistry staining suggests 3E10-D31N monoclonal antibody (V66) oligonucleotide conjugates utilizing transglutaminase-mediated enzymatic conjugation show greater uptake than 3E10-D31N monoclonal antibody (V66) oligonucleotide conjugated with lysine intermediate. These results are further exemplified in FIG. 40B, which show fold change in nuclei median fluorescence intensity (MFI) of all tested antibody oligonucleotide conjugate lots.

### Example 27: Treatment of Non-Small Cell Lung Cancer (NSCLC) with V66-MMAE in a

### Mouse Model

A study was conducted to investigate the safety and potency of a V66-MMAE conjugate in vivo for treating NSCLC in a mouse model. Briefly, a humanized 3E10 antibody with a heavy chain comprising an amino acid sequence of SEQ ID NO:76 and a light chain comprising the amino acid sequence of SEQ ID NO:96 (“V66”) was conjugated to MMAE through a mc-vc-PAB-MMAE linker covalently conjugated to free interchain cysteines at an average drug to antibody ratio of 3.0. BALB/c nude mice were subcutaneously implanted with human A427 NSCLC lung cancer cells and randomized for treatment once tumors measured 100-150 mm3. via intrathecal injection and Tumor bearing mice were subsequently treated with systemic (IP) administration of the V66-MMAE conjugate with a schedule of QWx4 (once weekly for four weeks), 2QWx4 (twice weekly for four weeks), or 3QWx4 (three times weekly for four weeks). Vehicle (PBS) was administered to animals 3QWx4 as control.

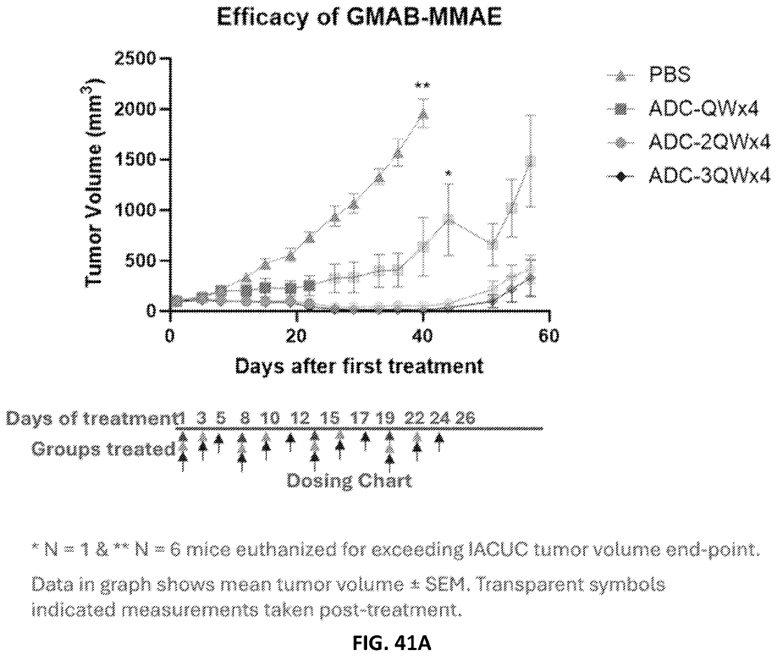

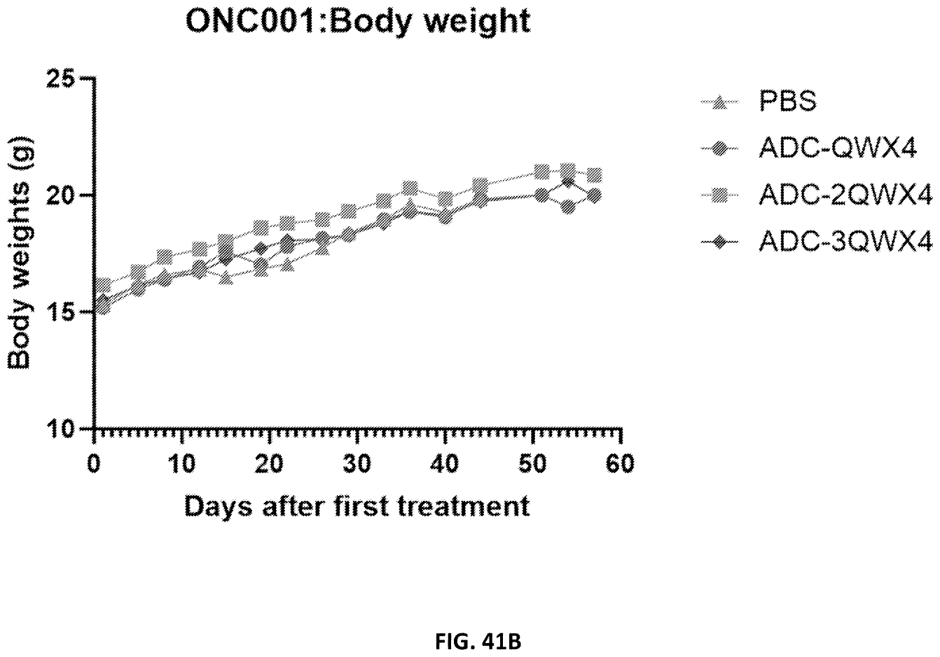

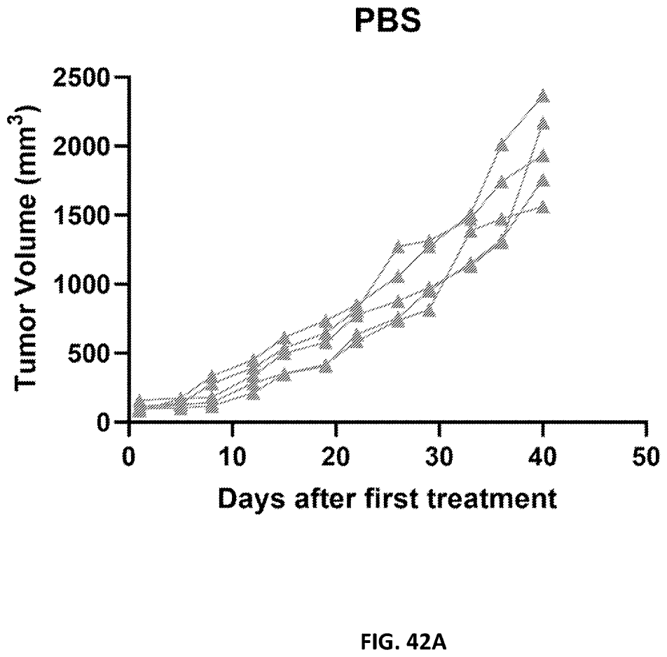

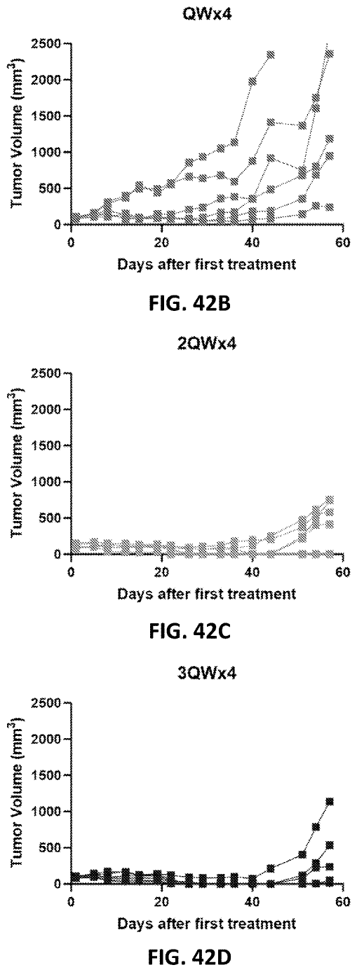

Tumor volume was measured over time post implantation. As shown in FIG. 41A and FIGS. 42A-C, tumor growth was reduced after treatment with V66-MMAE using any of the dosage schedules. On day 33 post last dose, 2/6 (33%) of mice were tumor free on the ADC-2QWx4 regimen. On day 31 post last dose, 1/6 (16%) of mice of mice were tumor free on the ADC-3QWx4 regimen.

Mouse weight was also monitored during the study period to identify detrimental effects of the treatment that would suggest non-specific toxicity to healthy tissue. FIG. 41B shows no changes in the weight of the mice treated with systemic administration of the V66-MMAE conjugate, as compared to the mice treated with PBS. Thus, V66-MMAE was very well tolerated.

This example demonstrates that systemic administration of the V66-MMAE conjugate post implantation reduces tumor growth in mice without evidence of non-specific toxicity.

### Example 28: In Vitro Potency of V66- Vc-Seco-DUBA ADCs Against 3 ENT2(+) Tumor Cell Lines

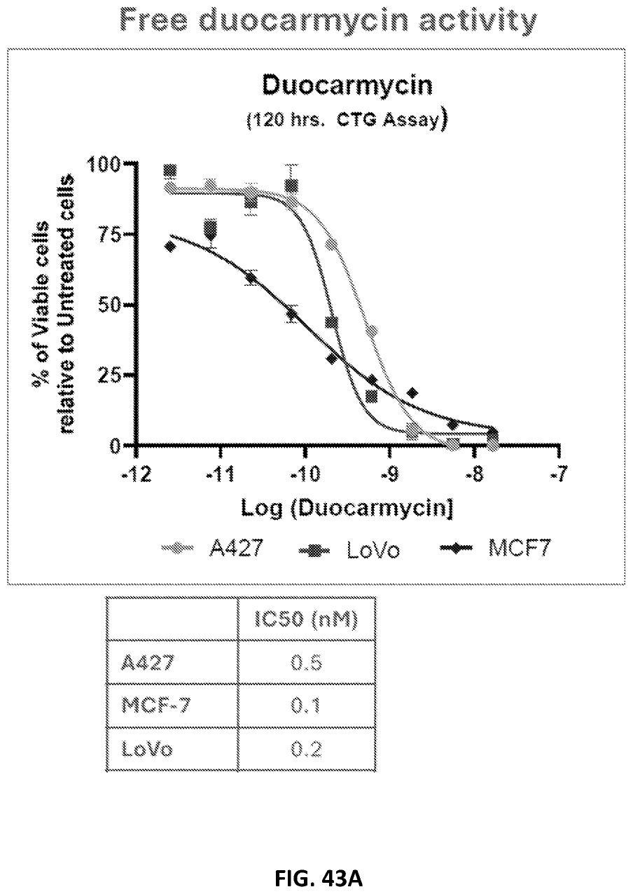

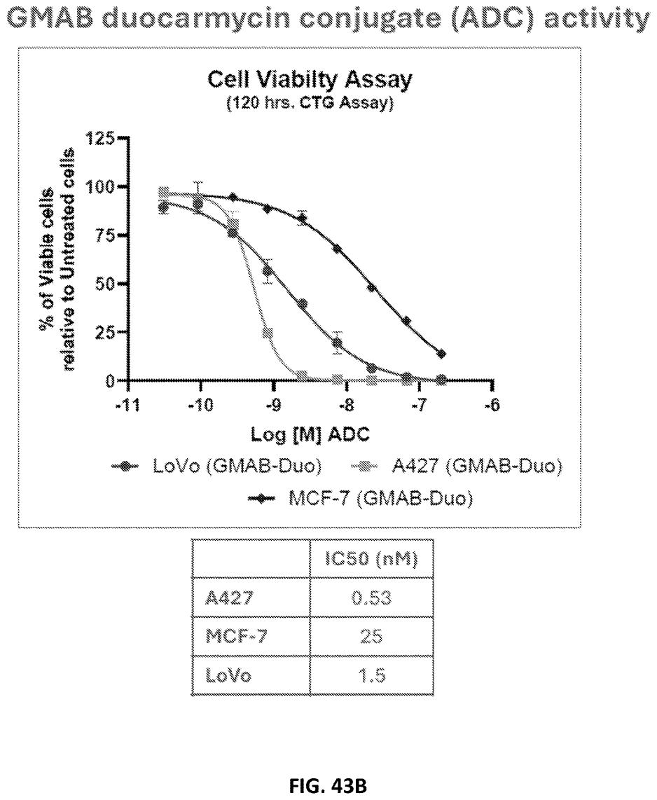

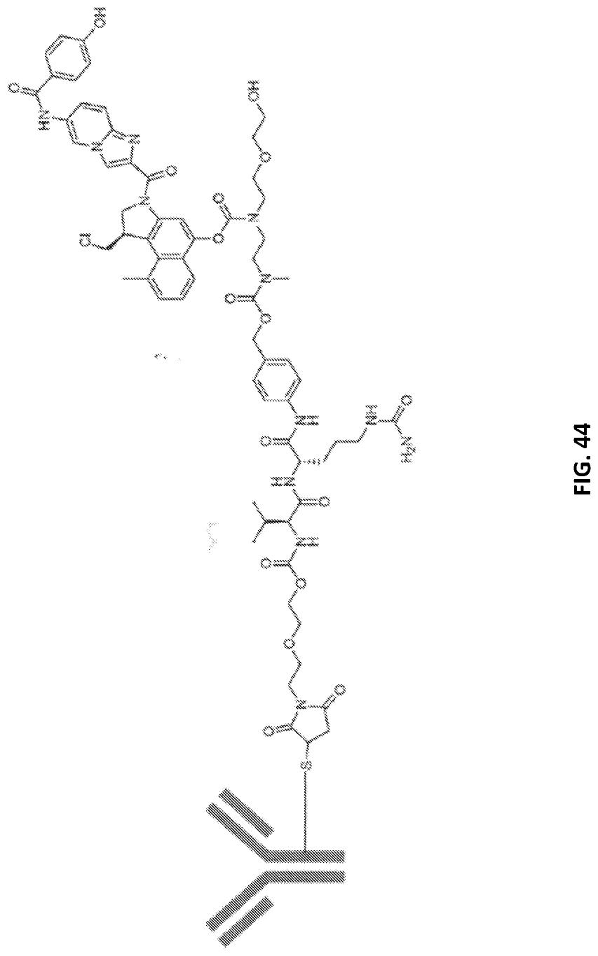

The effects of treatment with V66-vc-Seco-DUBA conjugates and DUBA alone in 3 ENT2(+) tumor cell lines (MCF7, LOVO, A427) were investigated. Seco-DUBA is a duocarmycin hydroxybenzamide azaindole (DUBA) prodrug containing two hydroxyl groups, which can each be used for coupling to an antibody via a linker. Seco-DUBA can be used in the synthesis of antibody-drug conjugates (ADCs). FIG. 43A-B shows the cell viability post-treatment with free DUBA and V66-vc-Seco-Duba in MCF7, LOVO, A427 cancer cell lines. Cells were treated with titrations of ADC or free drug and incubated for 120 hours. Cell viability determined by cell titer glo reagent. This example demonstrates that a 3E10 ADC DUBA exhibits potent cells killing in 3 ENT2(+) cell lines. There was an increased sensitivity of ADC in A427 lung model, with an IC50 similar to free payload.

Sequences

| SEQ | | | |
|---|---|---|---|
| ID | | | |
| NO. | Name | Description | Sequence |
| 1 | 3E10- | WT 3E10 antibody | EVQLVESGGGLVKPGGSRKLSCAASGFTFSDYGMHWV |
| | HC | sequences | RQAPEKGLEWVAYISSGSSTIYYADTVKGRFTISRDNA |
| | | | KNTLFLQMTSLRSEDTAMYYCARRGLLLDYWGQGTTL |
| | | | TVSAASTKGPSVFPLAPSSKSTSGGTAALGCLVKDYFPE |
| | | | PVTVSWNSGALTSGVHTFPAVLQSSGLYSLSSVVTVPSS |
| | | | SLGTQTYICNVNHKPSNTKVDKKVEPKSCDKTHTCPPC |
| | | | PAPELLGGPSVFLFPPKPKDTLMISRTPEVTCVVVDVSH |
| | | | EDPEVKFNWYVDGVEVHNAKTKPREEQYNSTYRVVS |
| | | | VLTVLHQDWLNGKEYKCKVSNKALPAPIEKTISKAKG |
| | | | QPREPQVYTLPPSRDELTKNQVSLTCLVKGFYPSDIAVE |
| | | | WESNGQPENNYKTTPPVLDSDGSFFLYSKLTVDKSRW |
| | | | QQGNVFSCSVMHEALHNHYTQKSLSLSPGK |
| 2 | 3E10- | WT 3E10 antibody | EVQLVESGGGLVKPGGSRKLSCAASGFTFSDYGMHWV |
| | VH | sequences | RQAPEKGLEWVAYISSGSSTIYYADTVKGRFTISRDNA |
| | | | KNTLFLQMTSLRSEDTAMYYCARRGLLLDYWGQGTTL |
| | | | TVSS |
| 3 | 3E10- | WT 3E10 antibody | DYGMH |
| | VH- | sequences | |
| | CDR1 | | |
| 4 | 3E10- | WT 3E10 antibody | YISSGSSTIYYADTVKG |
| | VH- | sequences | |
| | CDR2 | | |
| 5 | 3E10- | WT 3E10 antibody | RGLLLDY |
| | VH- | sequences | |
| | CDR3 | | |
| 6 | 3E10- | WT 3E10 antibody | MGWSCIILFLVATATGVHS |
| | HC-SP | sequences | |
| 7 | 3E10- | WT 3E10 antibody | DIVLTQSPASLAVSLGQRATISCRASKSVSTSSYSYMHW |
| | LC | sequences | YQQKPGQPPKLLIKYASYLESGVPARFSGSGSGTDFTLN |
| | | | IHPVEEEDAATYYCQHSREFPWTFGGGTKLEIKRTVAA |
| | | | PSVFIFPPSDEQLKSGTASVVCLLNNFYPREAKVQWKV |
| | | | DNALQSGNSQESVTEQDSKDSTYSLSSTLTLSKADYEK |
| | | | HKVYACEVTHQGLSSPVTKSFNRGEC |
| 8 | 3E10- | WT 3E10 antibody | DIVLTQSPASLAVSLGQRATISCRASKSVSTSSYSYMHW |
| | VL | sequences | YQQKPGQPPKLLIKYASYLESGVPARFSGSGSGTDFTLN |
| | | | IHPVEEEDAATYYCQHSREFPWTFGGGTKLEIK |
| 9 | 3E10- | WT 3E10 antibody | RASKSVSTSSYSYMH |
| | VL- | sequences | |
| | CDR1 | | |
| 10 | 3E10- | WT 3E10 antibody | YASYLES |
| | VL- | sequences | |
| | CDR2 | | |
| 11 | 3E10- | WT 3E10 antibody | QHSREFPWT |
| | VL- | sequences | |
| | CDR3 | | |
| 12 | 3E10- | WT 3E10 antibody | MGWSCIILFLVATATGVHS |
| | LC-SP | sequences | |
| 13 | 3E10- | D31N 3E10 | EVQLVESGGGLVKPGGSRKLSCAASGFTFSNYGMHWV |
| | HC | antibody sequences | RQAPEKGLEWVAYISSGSSTIYYADTVKGRFTISRDNA |
| | D31N | | KNTLFLQMTSLRSEDTAMYYCARRGLLLDYWGQGTTL |
| | | | TVSAASTKGPSVFPLAPSSKSTSGGTAALGCLVKDYFPE |
| | | | PVTVSWNSGALTSGVHTFPAVLQSSGLYSLSSVVTVPSS |
| | | | SLGTQTYICNVNHKPSNTKVDKKVEPKSCDKTHTCPPC |
| | | | PAPELLGGPSVFLFPPKPKDTLMISRTPEVTCVVVDVSH |
| | | | EDPEVKFNWYVDGVEVHNAKTKPREEQYNSTYRVVS |
| | | | VLTVLHQDWLNGKEYKCKVSNKALPAPIEKTISKAKG |
| | | | QPREPQVYTLPPSRDELTKNQVSLTCLVKGFYPSDIAVE |
| | | | WESNGQPENNYKTTPPVLDSDGSFFLYSKLTVDKSRW |
| | | | QQGNVFSCSVMHEALHNHYTQKSLSLSPGK |
| 14 | 3E10- | D31N 3E10 | EVQLVESGGGLVKPGGSRKLSCAASGFTFSNYGMHWV |
| | VH | antibody sequences | RQAPEKGLEWVAYISSGSSTIYYADTVKGRFTISRDNA |
| | D31N | | KNTLFLQMTSLRSEDTAMYYCARRGLLLDYWGQGTTL |
| | | | TVSS |
| 15 | 3E10- | D31N 3E10 | NYGMH |
| | VH- | antibody sequences | |
| | CDR1 | | |
| | D31N | | |
| 16 | 3E10- | D31N 3E10 | XYGMH, where X is D, N, R, or K |
| | VH- | antibody sequences | |
| | CDR1a | | |
| 17 | 3E10- | D31N 3E10 | YISSGSSTIYYADTVKG |
| | VH- | antibody sequences | |
| | CDR2 | | |
| | D31N | | |
| 18 | 3E10- | D31N 3E10 | RGLLLDY |
| | VH- | antibody sequences | |
| | CDR3 | | |
| | D31N | | |
| 19 | 3E10- | D31N 3E10 | MGWSCIILFLVATATGVHS |
| | HC-SP | antibody sequences | |
| | D31N | | |
| 20 | 3E10- | D31N 3E10 | DIVLTQSPASLAVSLGQRATISCRASKSVSTSSYSYMHW |
| | VL | antibody sequences | YQQKPGQPPKLLIKYASYLESGVPARFSGSGSGTDFTLN |
| | D31N | | IHPVEEEDAATYYCQHSREFPWTFGGGTKLEIKRTVAA |
| | | | PSVFIFPPSDEQLKSGTASVVCLLNNFYPREAKVQWKV |
| | | | DNALQSGNSQESVTEQDSKDSTYSLSSTLTLSKADYEK |
| | | | HKVYACEVTHQGLSSPVTKSFNRGEC |
| 21 | 3E10- | D31N 3E10 | DIVLTQSPASLAVSLGQRATISCRASKSVSTSSYSYMHW |
| | VL-VR | antibody sequences | YQQKPGQPPKLLIKYASYLESGVPARFSGSGSGTDFTLN |
| | D31N | | IHPVEEEDAATYYCQHSREFPWTFGGGTKLEIK |
| 22 | 3E10- | D31N 3E10 | RASKSVSTSSYSYMH |
| | VL- | antibody sequences | |
| | CDR1 | | |
| | D3IN | | |
| 23 | 3E10- | D31N 3E10 | YASYLES |
| | VL- | antibody sequences | |
| | CDR2 | | |
| | D31N | | |
| 24 | 3E10- | D31N 3E10 | QHSREFPWT |
| | VL- | antibody sequences | |
| | CDR3 | | |
| | D31N | | |
| 25 | 3E10- | D31N 3E10 | MGWSCIILFLVATATGVHS |
| | LC-SP | antibody sequences | |
| | D31N | | |
| 26 | 3E10- | Other known 3E10 | YISSGSSTIYYADSVKG |
| | VH- | CDR variants | |
| | CDR21 | | |
| 27 | 3E10- | Other known 3E10 | YISSSSSTIYYADSVKG |
| | VH- | CDR variants | |
| | CDR22 | | |
| 28 | 3E10- | Other known 3E10 | RASKSVSTSSYSYLA |
| | VL- | CDR variants | |
| | CDR1.1 | | |
| 29 | 3E10- | Other known 3E10 | RASKTVSTSSYSYMH |
| | VL- | CDR variants | |
| | CDR12 | | |
| 30 | 3E10- | Other known 3E10 | YASYLQS |
| | VL- | CDR variants | |
| | CDR21 | | |
| 31 | 3E10- | Additionally | YISSX1SSTIYY ADX2VKG, where: X1 and X2 are |
| | VH- | contemplated 3E10 | separately any amino acid |
| | CDR2.3 | CDR variants | |
| 32 | 3E10- | Additionally | RASKXIVSTSSYSYX2X3, where: X1, X2, and X3 and are |
| | VL- | contemplated 3E10 | separately any amino acid |
| | CDR1.3 | CDR variants | |
| 33 | 3E10- | Additionally | YASYLXIS, where: X1 is any amino acid |
| | VL- | contemplated 3E10 | |
| | CDR2.2 | CDR variants | |
| 34 | 3E10- | Charge-conserved | QYGMH |
| | VH- | Variant 3E10 CDRs | |
| | CDR1.c1 | | |
| 35 | 3E10- | Charge-conserved | EYGMH |
| | VH- | Variant 3E10 CDRs | |
| | CDR1.c2 | | |
| 36 | 3E10- | Charge-conserved | X1YGMX2, where: X1 is D or N, and X2 is K or R |
| | VH- | Variant 3E10 CDRs | |
| | CDR1.c3 | | |
| 37 | 3E10- | Charge-conserved | QYGMX₁, where X₁ is K or R |
| | VH- | Variant 3E10 CDRs | |
| | CDR1.c4 | | |
| 38 | 3E10- | Charge-conserved | EYGMX1, where X1 is K or R |
| | VH- | Variant 3E10 CDRs | |
| | CDR1.c5 | | |
| 39 | 3E10- | Charge-conserved | YISSGSSTIYYAETVKG |
| | VH- | Variant 3E10 CDRs | |
| | CDR2.c1 | | |
| 40 | 3E10- | Charge-conserved | YISSGSSTIYYADTVX₁G, where X₁ is R or H |
| | VH- | Variant 3E10 CDRs | |
| | CDR2.c2 | | |
| 41 | 3E10- | Charge-conserved | YISSGSSTIYYAETVX₁G, where X₁ is R or H |
| | VH- | Variant 3E10 CDRs | |
| | CDR2.c3 | | |
| 42 | 3E10- | Charge-conserved | X₁GLLLDY |
| | VH- | Variant 3E10 CDRs | |
| | CDR3.c1 | | |
| 43 | 3E10- | Charge-conserved | RGLLLEY |
| | VH- | Variant 3E10 CDRs | |
| | CDR3.c2 | | |
| 44 | 3E10- | Charge-conserved | X₁GLLLEY, where X₁ is K or H |
| | VH- | Variant 3E10 CDRs | |
| | CDR3.c3 | | |
| 45 | 3E10- | Charge-conserved | X₁ASKSVSTSSYSYMH, where X₁ is K or H |
| | VL- | Variant 3E10 CDRs | |
| | CDR1.c1 | | |
| 46 | 3E10- | Charge-conserved | RASX₁SVSTSSYSYMH, where X₁ is R or H |
| | VL- | Variant 3E10 CDRs | |
| | CDR1.c2 | | |
| 47 | 3E10- | Charge-conserved | RASKSVSTSSYSYMX₁, where X₁ is K or R |
| | VL- | Variant 3E10 CDRs | |
| | CDR1.c3 | | |
| 48 | 3E10- | Charge-conserved | X1ASX2SVSTSSYSYMH, where: |
| | VL- | Variant 3E10 CDRs | X1 is K or H, and |
| | CDR1.c4 | | X2 is R or H |
| 49 | 3E10- | Charge-conserved | X1ASKSVSTSSYSYMX2, where: |
| | VL- | Variant 3E10 CDRs | X1 is K or H, and |
| | CDR1.c5 | | X2 is K or R |
| 50 | 3E10- | Charge-conserved | RASX1SVSTSSYSYMX2, where: |
| | VL- | Variant 3E10 CDRs | X1 is R or H, and |
| | CDR1.c6 | | X2 is K or R |
| 51 | 3E10- | Charge-conserved | YASYLDS |
| | VL- | Variant 3E10 CDRs | |
| | CDR2.c1 | | |
| 52 | 3E10- | Charge-conserved | QX₁ SREFPWT, where X₁ is K or R |
| | VL- | Variant 3E10 CDRs | |
| | CDR3.c1 | | |
| 53 | 3E10- | Charge-conserved | QHSX₁EFPWT, where X1 is K or H |
| | VL- | Variant 3E10 CDRs | |
| | CDR3.c2 | | |
| 54 | 3E10- | Charge-conserved | QHSRDFPWT |
| | VL- | Variant 3E10 CDRs | |
| | CDR3.c3 | | |
| 55 | 3E10- | Charge-conserved | QX1SX2EFPWT, where: |
| | VL- | Variant 3E10 CDRs | X1 is K or R, and |
| | CDR3.c4 | | X2 is K or H |
| 56 | 3E10- | Charge-conserved | QX₁SRDFPWT, where X₁ is K or R |
| | VL- | Variant 3E10 CDRs | |
| | CDR3.c5 | | |
| 57 | 3E10- | Charge-conserved | QHSX₁DFPWT, where X₁ is K or H |
| | VL- | Variant 3E10 CDRs | |
| | CDR3.c6 | | |
| 58 | 3E10- | Compound Variant | X1YGMX2, where: |
| | VH- | 3E10 CDRs | X1 is D, E, N, Q, R, or K and |
| | CDR1m | | X2 is K, R, or H |
| 59 | 3E10- | Compound Variant | YISSX1SSTIYYAX2X3VX4G, where: |
| | VH- | 3E10 CDRs | X1 is G or S, |
| | CDR2m | | X2 is D or E, |
| | | | X3 is T or S, and |
| | | | X4 is K, R, or H |
| 60 | 3E10- | Compound Variant | X1GLLLX2Y, where: |
| | VH- | 3E10 CDRs | X1 is K, R, or H, and |
| | CDR3m | | X2 is D or E |
| 61 | 3E10- | Compound Variant | X1ASX2X3VSTSSYSYX4X5, where: |
| | VL- | 3E10 CDRs | X1 is K, R, or H, |
| | CDR1m | | X2 is K, R, or H, |
| | | | X3 is T or s, |
| | | | X4 is M or L, and |
| | | | X5 is K, R, H, or A |
| 62 | 3E10- | Compound Variant | YASYLXIS, where: |
| | VL- | 3E10 CDRs | X1 is D, E, N, or Q |
| | CDR2m | | |
| 63 | 3E10- | Compound Variant | QX1SX2X3FPWT, where: |
| | VL- | 3E10 CDRs | X1 is K, R, or H, |
| | CDR3m | | X2 is K, R, or H, and |
| | | | X3 is D or E |
| 64 | 3E10- | Humanized 3E10 | EVQLVESGGGLVQPGGSLRLSCAASGFTFSNYGMHWV |
| | VH- | Heavy Chain | RQAPGKGLEWVSYISSGSSTIYYADTVKGRFTISRDNAK |
| | h1 | Variable Regions | NSLYLQMNSLRAEDTAVYYCARRGLLLDYWGQGTLV |
| | | | TVSS |
| 65 | 3E10- | Humanized 3E10 | EVQLVESGGGLVQPGGSLRLSCAASGFTFSNYGMHWVRQAP |
| | VH- | Heavy Chain | GKGLEWVAYISSGSSTIYYADTVKGRFTISRDNAKNSLYLQM |
| | h2 | Variable Regions | NSLRAEDTAMYYCARRGLLLDYWGQGTLVTVSS |
| 66 | 3E10- | Humanized 3E10 | EVQLVESGGGLVQPGGSLRLSCAASGFTFSNYGMHWVRQAP |
| | VH- | Heavy Chain | GKGLEWVAYISSGSSTIYYADTVKGRFTISRDNAKNSLYLQM |
| | h3 | Variable Regions | TSLRAEDTAMYYCARRGLLLDYWGQGTLVTVSS |
| 67 | 3E10- | Humanized 3E10 | EVQLVESGGGLVQPGGSLRLSCAASGFTFSNYGMHWVRQAP |
| | VH- | Heavy Chain | EKGLEWVAYISSGSSTIYYADTVKGRFTISRDNAKNSLYLQM |
| | h4 | Variable Regions | TSLRAEDTAMYYCARRGLLLDYWGQGTLVTVSS |
| 68 | 3E10- | Humanized 3E10 | EVQLVESGGGLVQPGGSLRLSCAASGFTFSNYGMHWVRQAP |
| | VH- | Heavy Chain | GKGLEWVAYISSGSSTIYYADTVKGRFTISRDNAKNTLFLQM |
| | h5 | Variable Regions | NSLRAEDTAVYYCARRGLLLDYWGQGTLVTVSS |
| 69 | 3E10- | Humanized 3E10 | EVQLVESGGGLVQPGGSRKLSCAASGFTFSNYGMHWVRQAP |
| | VH- | Heavy Chain | GKGLEWVAYISSGSSTIYYADTVKGRFTISRDNAKNTLFLQM |
| | h6 | Variable Regions | NSLRAEDTAVYYCARRGLLLDYWGQGTLVTVSS |
| 70 | 3E10- | Humanized 3E10 | EVQLVESGGGLVQPGGSRKLSCAASGFTFSNYGMHWVRQAP |
| | VH- | Heavy Chain | EKGLEWVAYISSGSSTIYYADTVKGRFTISRDNAKNTLFLQM |
| | h7 | Variable Regions | TSLRAEDTAVYYCARRGLLLDYWGQGTLVTVSS |
| 71 | 3E10- | Humanized 3E10 | EVQLVESGGGLVQPGGSLRLSCAASGFTFSNYGMHWVRQAP |
| | HC- | Heavy Chain | GKGLEWVSYISSGSSTIYYADTVKGRFTISRDNAKNSLYLQM |
| | h1m | (without signal | NSLRAEDTAVYYCARRGLLLDYWGQGTLVTVSSASTKGPSV |
| | | peptide) | FPLAPSSKSTSGGTAALGCLVKDYFPEPVTVSWNSGALTSGV |
| | | | HTFPAVLQSSGLYSLSSVVTVPSSSLGTQTYICNVNHKPSNTK |
| | | | VDKKVEPKSCDKTHTCPPCPAPELLGGPSVFLFPPKPKDTLMI |
| | | | SRTPEVTCVVVDVSHEDPEVKFNWYVDGVEVHNAKTKPREE |
| | | | QYNSTYRVVSVLTVLHQDWLNGKEYKCKVSNKALPAPIEKT |
| | | | ISKAKGQPREPQVYTLPPSRDELTKNQVSLTCLVKGFYPSDIA |
| | | | VEWESNGQPENNYKTTPPVLDSDGSFFLYSKLTVDKSRWQQ |
| | | | GNVFSCSVMHEALHNHYTQKSLSLSPGK |
| 72 | 3E10- | Humanized 3E10 | EVQLVESGGGLVQPGGSLRLSCAASGFTFSNYGMHWVRQAP |
| | HC- | Heavy Chain | GKGLEWVAYISSGSSTIYYADTVKGRFTISRDNAKNSLYLQM |
| | h2m | (without signal | NSLRAEDTAMYYCARRGLLLDYWGQGTLVTVSSASTKGPSV |
| | | peptide) | FPLAPSSKSTSGGTAALGCLVKDYFPEPVTVSWNSGALTSGV |
| | | | HTFPAVLQSSGLYSLSSVVTVPSSSLGTQTYICNVNHKPSNTK |
| | | | VDKKVEPKSCDKTHTCPPCPAPELLGGPSVFLFPPKPKDTLMI |
| | | | SRTPEVTCVVVDVSHEDPEVKFNWYVDGVEVHNAKTKPREE |
| | | | QYNSTYRVVSVLTVLHQDWLNGKEYKCKVSNKALPAPIEKT |
| | | | ISKAKGQPREPQVYTLPPSRDELTKNQVSLTCLVKGFYPSDIA |
| | | | VEWESNGQPENNYKTTPPVLDSDGSFFLYSKLTVDKSRWQQ |
| | | | GNVFSCSVMHEALHNHYTQKSLSLSPGK |
| 73 | 3E10- | Humanized 3E10 | EVQLVESGGGLVQPGGSLRLSCAASGFTFSNYGMHWVRQAP |
| | HC- | Heavy Chain | GKGLEWVAYISSGSSTIYYADTVKGRFTISRDNAKNSLYLQM |
| | h3m | (without signal | TSLRAEDTAMYYCARRGLLLDYWGQGTLVTVSSASTKGPSV |
| | | peptide) | FPLAPSSKSTSGGTAALGCLVKDYFPEPVTVSWNSGALTSGV |
| | | | HTFPAVLQSSGLYSLSSVVTVPSSSLGTQTYICNVNHKPSNTK |
| | | | VDKKVEPKSCDKTHTCPPCPAPELLGGPSVFLFPPKPKDTLMI |
| | | | SRTPEVTCVVVDVSHEDPEVKFNWYVDGVEVHNAKTKPREE |
| | | | QYNSTYRVVSVLTVLHQDWLNGKEYKCKVSNKALPAPIEKT |
| | | | ISKAKGQPREPQVYTLPPSRDELTKNQVSLTCLVKGFYPSDIA |
| | | | VEWESNGQPENNYKTTPPVLDSDGSFFLYSKLTVDKSRWQQ |
| | | | GNVFSCSVMHEALHNHYTQKSLSLSPGK |
| 74 | 3E10- | Humanized 3E10 | EVQLVESGGGLVQPGGSLRLSCAASGFTFSNYGMHWVRQAP |
| | HC- | Heavy Chain | EKGLEWVAYISSGSSTIYYADTVKGRFTISRDNAKNSLYLQM |
| | h4m | (without signal | TSLRAEDTAMYYCARRGLLLDYWGQGTLVTVSSASTKGPSV |
| | | peptide) | FPLAPSSKSTSGGTAALGCLVKDYFPEPVTVSWNSGALTSGV |
| | | | HTFPAVLQSSGLYSLSSVVTVPSSSLGTQTYICNVNHKPSNTK |
| | | | VDKKVEPKSCDKTHTCPPCPAPELLGGPSVFLFPPKPKDTLMI |
| | | | SRTPEVTCVVVDVSHEDPEVKFNWYVDGVEVHNAKTKPREE |
| | | | QYNSTYRVVSVLTVLHQDWLNGKEYKCKVSNKALPAPIEKT |
| | | | ISKAKGQPREPQVYTLPPSRDELTKNQVSLTCLVKGFYPSDIA |
| | | | VEWESNGQPENNYKTTPPVLDSDGSFFLYSKLTVDKSRWQQ |
| | | | GNVFSCSVMHEALHNHYTQKSLSLSPGK |
| 75 | 3E10- | Humanized 3E10 | EVQLVESGGGLVQPGGSLRLSCAASGFTFSNYGMHWVRQAP |
| | HC- | Heavy Chain | GKGLEWVAYISSGSSTIYYADTVKGRFTISRDNAKNTLFLQM |
| | h5m | (without signal | NSLRAEDTAVYYCARRGLLLDYWGQGTLVTVSSASTKGPSV |
| | | peptide) | FPLAPSSKSTSGGTAALGCLVKDYFPEPVTVSWNSGALTSGV |
| | | | HTFPAVLQSSGLYSLSSVVTVPSSSLGTQTYICNVNHKPSNTK |
| | | | VDKKVEPKSCDKTHTCPPCPAPELLGGPSVFLFPPKPKDTLMI |
| | | | SRTPEVTCVVVDVSHEDPEVKFNWYVDGVEVHNAKTKPREE |
| | | | QYNSTYRVVSVLTVLHQDWLNGKEYKCKVSNKALPAPIEKT |
| | | | ISKAKGQPREPQVYTLPPSRDELTKNQVSLTCLVKGFYPSDIA |
| | | | VEWESNGQPENNYKTTPPVLDSDGSFFLYSKLTVDKSRWQQ |
| | | | GNVFSCSVMHEALHNHYTQKSLSLSPGK |
| 76 | 3E10- | Humanized 3E10 | EVQLVESGGGLVQPGGSRKLSCAASGFTFSNYGMHWVRQAP |
| | HC- | Heavy Chain | GKGLEWVAYISSGSSTIYYADTVKGRFTISRDNAKNTLFLQM |
| | h6m | (without signal | NSLRAEDTAVYYCARRGLLLDYWGQGTLVTVSSASTKGPSV |
| | | peptide) | FPLAPSSKSTSGGTAALGCLVKDYFPEPVTVSWNSGALTSGV |
| | | | HTFPAVLQSSGLYSLSSVVTVPSSSLGTQTYICNVNHKPSNTK |
| | | | VDKKVEPKSCDKTHTCPPCPAPELLGGPSVFLFPPKPKDTLMI |
| | | | SRTPEVTCVVVDVSHEDPEVKFNWYVDGVEVHNAKTKPREE |
| | | | QYNSTYRVVSVLTVLHQDWLNGKEYKCKVSNKALPAPIEKT |
| | | | ISKAKGQPREPQVYTLPPSRDELTKNQVSLTCLVKGFYPSDIA |
| | | | VEWESNGQPENNYKTTPPVLDSDGSFFLYSKLTVDKSRWQQ |
| | | | GNVFSCSVMHEALHNHYTQKSLSLSPGK |
| 77 | 3E10- | Humanized 3E10 | EVQLVESGGGLVQPGGSRKLSCAASGFTFSNYGMHWVRQAP |
| | HC- | Heavy Chain | EKGLEWVAYISSGSSTIYYADTVKGRFTISRDNAKNTLFLQM |
| | h7m | (without signal | TSLRAEDTAVYYCARRGLLLDYWGQGTLVTVSSASTKGPSV |
| | | peptide) | FPLAPSSKSTSGGTAALGCLVKDYFPEPVTVSWNSGALTSGV |
| | | | HTFPAVLQSSGLYSLSSVVTVPSSSLGTQTYICNVNHKPSNTK |
| | | | VDKKVEPKSCDKTHTCPPCPAPELLGGPSVFLFPPKPKDTLMI |
| | | | SRTPEVTCVVVDVSHEDPEVKFNWYVDGVEVHNAKTKPREE |
| | | | QYNSTYRVVSVLTVLHQDWLNGKEYKCKVSNKALPAPIEKT |
| | | | ISKAKGQPREPQVYTLPPSRDELTKNQVSLTCLVKGFYPSDIA |
| | | | VEWESNGQPENNYKTTPPVLDSDGSFFLYSKLTVDKSRWQQ |
| | | | GNVFSCSVMHEALHNHYTQKSLSLSPGK |
| 78 | 3E10- | Humanized 3E10 | MGWSCIILFLVATATGVHSEVQLVESGGGLVQPGGSLRLSCA |
| | HC- | Heavy Chain (with | ASGFTFSNYGMHWVRQAPGKGLEWVSYISSGSSTIYYADTV |
| | h1 | signal peptide) | KGRFTISRDNAKNSLYLQMNSLRAEDTAVYYCARRGLLLDY |
| | | | WGQGTLVTVSSASTKGPSVFPLAPSSKSTSGGTAALGCLVKD |
| | | | YFPEPVTVSWNSGALTSGVHTFPAVLQSSGLYSLSSVVTVPSS |
| | | | SLGTQTYICNVNHKPSNTKVDKKVEPKSCDKTHTCPPCPAPE |
| | | | LLGGPSVFLFPPKPKDTLMISRTPEVTCVVVDVSHEDPEVKFN |
| | | | WYVDGVEVHNAKTKPREEQYNSTYRVVSVLTVLHQDWLNG |
| | | | KEYKCKVSNKALPAPIEKTISKAKGQPREPQVYTLPPSRDELT |
| | | | KNQVSLTCLVKGFYPSDIAVEWESNGQPENNYKTTPPVLDSD |
| | | | GSFFLYSKLTVDKSRWQQGNVFSCSVMHEALHNHYTQKSLS |
| | | | LSPGK |
| 79 | 3E10- | Humanized 3E10 | MGWSCIILFLVATATGVHSEVQLVESGGGLVQPGGSLRLSCA |
| | HC | Heavy Chain (with | ASGFTFSNYGMHWVRQAPGKGLEWVAYISSGSSTIYYADTV |
| | h2 | signal peptide) | KGRFTISRDNAKNSLYLQMNSLRAEDTAMYYCARRGLLLDY |
| | | | WGQGTLVTVSSASTKGPSVFPLAPSSKSTSGGTAALGCLVKD |
| | | | YFPEPVTVSWNSGALTSGVHTFPAVLQSSGLYSLSSVVTVPSS |
| | | | SLGTQTYICNVNHKPSNTKVDKKVEPKSCDKTHTCPPCPAPE |
| | | | LLGGPSVFLFPPKPKDTLMISRTPEVTCVVVDVSHEDPEVKFN |
| | | | WYVDGVEVHNAKTKPREEQYNSTYRVVSVLTVLHQDWLNG |
| | | | KEYKCKVSNKALPAPIEKTISKAKGQPREPQVYTLPPSRDELT |
| | | | KNQVSLTCLVKGFYPSDIAVEWESNGQPENNYKTTPPVLDSD |
| | | | GSFFLYSKLTVDKSRWQQGNVFSCSVMHEALHNHYTQKSLS |
| | | | LSPGK |
| 80 | 3E10- | Humanized 3E10 | MGWSCIILFLVATATGVHSEVQLVESGGGLVQPGGSLRLSCA |
| | HC- | Heavy Chain (with | ASGFTFSNYGMHWVRQAPGKGLEWVAYISSGSSTIYYADTV |
| | h3 | signal peptide) | KGRFTISRDNAKNSLYLQMTSLRAEDTAMYYCARRGLLLDY |
| | | | WGQGTLVTVSSASTKGPSVFPLAPSSKSTSGGTAALGCLVKD |
| | | | YFPEPVTVSWNSGALTSGVHTFPAVLQSSGLYSLSSVVTVPSS |
| | | | SLGTQTYICNVNHKPSNTKVDKKVEPKSCDKTHTCPPCPAPE |
| | | | LLGGPSVFLFPPKPKDTLMISRTPEVTCVVVDVSHEDPEVKFN |
| | | | WYVDGVEVHNAKTKPREEQYNSTYRVVSVLTVLHQDWLNG |
| | | | KEYKCKVSNKALPAPIEKTISKAKGQPREPQVYTLPPSRDELT |
| | | | KNQVSLTCLVKGFYPSDIAVEWESNGQPENNYKTTPPVLDSD |
| | | | GSFFLYSKLTVDKSRWQQGNVFSCSVMHEALHNHYTQKSLS |
| | | | LSPGK |
| 81 | 3E10- | Humanized 3E10 | MGWSCIILFLVATATGVHSEVQLVESGGGLVQPGGSLRLSCA |
| | HC- | Heavy Chain (with | ASGFTFSNYGMHWVRQAPEKGLEWVAYISSGSSTIYYADTV |
| | h4 | signal peptide) | KGRFTISRDNAKNSLYLQMTSLRAEDTAMYYCARRGLLLDY |
| | | | WGQGTLVTVSSASTKGPSVFPLAPSSKSTSGGTAALGCLVKD |
| | | | YFPEPVTVSWNSGALTSGVHTFPAVLQSSGLYSLSSVVTVPSS |
| | | | SLGTQTYICNVNHKPSNTKVDKKVEPKSCDKTHTCPPCPAPE |
| | | | LLGGPSVFLFPPKPKDTLMISRTPEVTCVVVDVSHEDPEVKFN |
| | | | WYVDGVEVHNAKTKPREEQYNSTYRVVSVLTVLHQDWLNG |
| | | | KEYKCKVSNKALPAPIEKTISKAKGQPREPQVYTLPPSRDELT |
| | | | KNQVSLTCLVKGFYPSDIAVEWESNGQPENNYKTTPPVLDSD |
| | | | GSFFLYSKLTVDKSRWQQGNVFSCSVMHEALHNHYTQKSLS |
| | | | LSPGK |
| 82 | 3E10- | Humanized 3E10 | MGWSCIILFLVATATGVHSEVQLVESGGGLVQPGGSLRLSCA |
| | HC- | Heavy Chain (with | ASGFTFSNYGMHWVRQAPGKGLEWVAYISSGSSTIYYADTV |
| | h5 | signal peptide) | KGRFTISRDNAKNTLFLQMNSLRAEDTAVYYCARRGLLLDY |
| | | | WGQGTLVTVSSASTKGPSVFPLAPSSKSTSGGTAALGCLVKD |
| | | | YFPEPVTVSWNSGALTSGVHTFPAVLQSSGLYSLSSVVTVPSS |
| | | | SLGTQTYICNVNHKPSNTKVDKKVEPKSCDKTHTCPPCPAPE |
| | | | LLGGPSVFLFPPKPKDTLMISRTPEVTCVVVDVSHEDPEVKFN |
| | | | WYVDGVEVHNAKTKPREEQYNSTYRVVSVLTVLHQDWLNG |
| | | | KEYKCKVSNKALPAPIEKTISKAKGQPREPQVYTLPPSRDELT |
| | | | KNQVSLTCLVKGFYPSDIAVEWESNGQPENNYKTTPPVLDSD |
| | | | GSFFLYSKLTVDKSRWQQGNVFSCSVMHEALHNHYTQKSLS |
| | | | LSPGK |
| 83 | 3E10- | Humanized 3E10 | MGWSCIILFLVATATGVHSEVQLVESGGGLVQPGGSRKLSCA |
| | HC- | Heavy Chain (with | ASGFTFSNYGMHWVRQAPGKGLEWVAYISSGSSTIYYADTV |
| | h6 | signal peptide) | KGRFTISRDNAKNTLFLQMNSLRAEDTAVYYCARRGLLLDY |
| | | | WGQGTLVTVSSASTKGPSVFPLAPSSKSTSGGTAALGCLVKD |
| | | | YFPEPVTVSWNSGALTSGVHTFPAVLQSSGLYSLSSVVTVPSS |
| | | | SLGTQTYICNVNHKPSNTKVDKKVEPKSCDKTHTCPPCPAPE |
| | | | LLGGPSVFLFPPKPKDTLMISRTPEVTCVVVDVSHEDPEVKFN |
| | | | WYVDGVEVHNAKTKPREEQYNSTYRVVSVLTVLHQDWLNG |
| | | | KEYKCKVSNKALPAPIEKTISKAKGQPREPQVYTLPPSRDELT |
| | | | KNQVSLTCLVKGFYPSDIAVEWESNGQPENNYKTTPPVLDSD |
| | | | GSFFLYSKLTVDKSRWQQGNVFSCSVMHEALHNHYTQKSLS |
| | | | LSPGK |
| 84 | 3E10- | Humanized 3E10 | MGWSCIILFLVATATGVHSEVQLVESGGGLVQPGGSRKLSCA |
| | HC- | Heavy Chain (with | ASGFTFSNYGMHWVRQAPEKGLEWVAYISSGSSTIYYADTV |
| | h7 | signal peptide) | KGRFTISRDNAKNTLFLQMTSLRAEDTAVYYCARRGLLLDY |
| | | | WGQGTLVTVSSASTKGPSVFPLAPSSKSTSGGTAALGCLVKD |
| | | | YFPEPVTVSWNSGALTSGVHTFPAVLQSSGLYSLSSVVTVPSS |
| | | | SLGTQTYICNVNHKPSNTKVDKKVEPKSCDKTHTCPPCPAPE |
| | | | LLGGPSVFLFPPKPKDTLMISRTPEVTCVVVDVSHEDPEVKFN |
| | | | WYVDGVEVHNAKTKPREEQYNSTYRVVSVLTVLHQDWLNG |
| | | | KEYKCKVSNKALPAPIEKTISKAKGQPREPQVYTLPPSRDELT |
| | | | KNQVSLTCLVKGFYPSDIAVEWESNGQPENNYKTTPPVLDSD |
| | | | GSFFLYSKLTVDKSRWQQGNVFSCSVMHEALHNHYTQKSLS |
| | | | LSPGK |
| 85 | VL1- | Humanized 3E10 | DIVLTQSPASLAVSPGQRATITCRASKSVSTSSYSYMHWYQQ |
| | C | Light Chain | KPGQPPKLLIYYASYLESGVPARFSGSGSGTDFTLTINPVEAN |
| | | Variable Regions | DTANYYCQHSREFPWTFGGGTKVEIK |
| 86 | VL2- | Humanized 3E10 | DIVLTQSPASLAVSPGQRATITCRASKSVSTSSYSYMHWYQQ |
| | C | Light Chain | KPGQPPKLLIKYASYLESGVPARFSGSGSGTDFTLTINPVEAE |
| | | Variable Regions | DTATYYCQHSREFPWTFGGGTKVEIK |
| 87 | VL3- | Humanized 3E10 | DIVLTQSPASLAVSPGQRATITCRASKSVSTSSYSYMHWYQQ |
| | C | Light Chain | KPGQPPKLLIKYASYLESGVPARFSGSGSGTDFTLTINPVEAE |
| | | Variable Regions | DTANYYCQHSREFPWTFGGGTKVEIK |
| 88 | VL4- | Humanized 3E10 | DIVLTQSPASLAVSPGQRATITCRASKSVSTSSYSYMHWYQQ |
| | C | Light Chain | KPGQPPKLLIKYASYLESGVPARFSGSGSGTDFTLTINPVEAE |
| | | Variable Regions | DAATYYCQHSREFPWTFGGGTKVEIK |
| 89 | VL5- | Humanized 3E10 | DIVLTQSPASLAVSPGQRATITCRASKSVSTSSYSYMHWYQQ |
| | C | Light Chain | KPGQPPKLLIKYASYLESGVPARFSGSGSGTDFTLNIHPVEAE |
| | | Variable Regions | DTANYYCQHSREFPWTFGGGTKVEIK |
| 90 | VL6- | Humanized 3E10 | DIVLTQSPASLAVSPGQRATISCRASKSVSTSSYSYMHWYQQ |
| | C | Light Chain | KPGQPPKLLIKYASYLESGVPARFSGSGSGTDFTLNIHPVEEE |
| | | Variable Regions | DTANYYCQHSREFPWTFGGGTKVEIK |
| 91 | 3E10- | Humanized 3E10 | DIVLTQSPASLAVSPGQRATITCRASKSVSTSSYSYMHWYQQ |
| | LC- | Light Chain | KPGQPPKLLIYYASYLESGVPARFSGSGSGTDFTLTINPVEAN |
| | h1m | (without signal | DTANYYCQHSREFPWTFGGGTKVEIKRTVAAPSVFIFPPSDE |
| | | peptide) | QLKSGTASVVCLLNNFYPREAKVQWKVDNALQSGNSQESVT |
| | | | EQDSKDSTYSLSSTLTLSKADYEKHKVYACEVTHQGLSSPVT |
| | | | KSFNRGEC |
| 92 | 3E10- | Humanized 3E10 | DIVLTQSPASLAVSPGQRATITCRASKSVSTSSYSYMHWYQQ |
| | LC- | Light Chain | KPGQPPKLLIKYASYLESGVPARFSGSGSGTDFTLTINPVEAE |
| | h2m | (without signal | DTATYYCQHSREFPWTFGGGTKVEIKRTVAAPSVFIFPPSDEQ |
| | | peptide) | LKSGTASVVCLLNNFYPREAKVQWKVDNALQSGNSQESVTE |
| | | | QDSKDSTYSLSSTLTLSKADYEKHKVYACEVTHQGLSSPVTK |
| | | | SFNRGEC |
| 93 | 3E10- | Humanized 3E10 | DIVLTQSPASLAVSPGQRATITCRASKSVSTSSYSYMHWYQQ |
| | LC- | Light Chain | KPGQPPKLLIKYASYLESGVPARFSGSGSGTDFTLTINPVEAE |
| | h3m | (without signal | DTANYYCQHSREFPWTFGGGTKVEIKRTVAAPSVFIFPPSDE |
| | | peptide) | QLKSGTASVVCLLNNFYPREAKVQWKVDNALQSGNSQESVT |
| | | | EQDSKDSTYSLSSTLTLSKADYEKHKVYACEVTHQGLSSPVT |
| | | | KSFNRGEC |
| 94 | 3E10- | Humanized 3E10 | DIVLTQSPASLAVSPGQRATITCRASKSVSTSSYSYMHWYQQ |
| | LC- | Light Chain | KPGQPPKLLIKYASYLESGVPARFSGSGSGTDFTLTINPVEAE |
| | h4m | (without signal | DAATYYCQHSREFPWTFGGGTKVEIKRTVAAPSVFIFPPSDE |
| | | peptide) | QLKSGTASVVCLLNNFYPREAKVQWKVDNALQSGNSQESVT |
| | | | EQDSKDSTYSLSSTLTLSKADYEKHKVYACEVTHQGLSSPVT |
| | | | KSFNRGEC |
| 95 | 3E10- | Humanized 3E10 | DIVLTQSPASLAVSPGQRATITCRASKSVSTSSYSYMHWYQQ |
| | LC- | Light Chain | KPGQPPKLLIKYASYLESGVPARFSGSGSGTDFTLNIHPVEAE |
| | h5m | (without signal | DTANYYCQHSREFPWTFGGGTKVEIKRTVAAPSVFIFPPSDE |
| | | peptide) | QLKSGTASVVCLLNNFYPREAKVQWKVDNALQSGNSQESVT |
| | | | EQDSKDSTYSLSSTLTLSKADYEKHKVYACEVTHQGLSSPVT |
| | | | KSFNRGEC |
| 96 | 3E10- | Humanized 3E10 | DIVLTQSPASLAVSPGQRATISCRASKSVSTSSYSYMHWYQQ |
| | LC- | Light Chain | KPGQPPKLLIKYASYLESGVPARFSGSGSGTDFTLNIHPVEEE |
| | h6m | (without signal | DTANYYCQHSREFPWTFGGGTKVEIKRTVAAPSVFIFPPSDE |
| | | peptide) | QLKSGTASVVCLLNNFYPREAKVQWKVDNALQSGNSQESVT |
| | | | EQDSKDSTYSLSSTLTLSKADYEKHKVYACEVTHQGLSSPVT |
| | | | KSFNRGEC |
| 97 | 3E10- | Humanized 3E10 | MGWSCIILFLVATATGVHSDIVLTQSPASLAVSPGQRATITCR |
| | LC-h1 | Light Chain (with | ASKSVSTSSYSYMHWYQQKPGQPPKLLIYYASYLESGVPARF |
| | | signal peptide) | SGSGSGTDFTLTINPVEANDTANYYCQHSREFPWTFGGGTKV |
| | | | EIKRTVAAPSVFIFPPSDEQLKSGTASVVCLLNNFYPREAKVQ |
| | | | WKVDNALQSGNSQESVTEQDSKDSTYSLSSTLTLSKADYEK |
| | | | HKVYACEVTHQGLSSPVTKSFNRGEC |
| 98 | 3E10- | Humanized 3E10 | MGWSCIILFLVATATGVHSDIVLTQSPASLAVSPGQRATITCR |
| | LC-h2 | Light Chain (with | ASKSVSTSSYSYMHWYQQKPGQPPKLLIKYASYLESGVPARF |
| | | signal peptide) | SGSGSGTDFTLTINPVEAEDTATYYCQHSREFPWTFGGGTKV |
| | | | EIKRTVAAPSVFIFPPSDEQLKSGTASVVCLLNNFYPREAKVQ |
| | | | WKVDNALQSGNSQESVTEQDSKDSTYSLSSTLTLSKADYEK |
| | | | HKVYACEVTHQGLSSPVTKSFNRGEC |
| 99 | 3E10- | Humanized 3E10 | MGWSCIILFLVATATGVHSDIVLTQSPASLAVSPGQRATITCR |
| | LC-h3 | Light Chain (with | ASKSVSTSSYSYMHWYQQKPGQPPKLLIKYASYLESGVPARF |
| | | signal peptide) | SGSGSGTDFTLTINPVEAEDTANYYCQHSREFPWTFGGGTKV |
| | | | EIKRTVAAPSVFIFPPSDEQLKSGTASVVCLLNNFYPREAKVQ |
| | | | WKVDNALQSGNSQESVTEQDSKDSTYSLSSTLTLSKADYEK |
| | | | HKVYACEVTHQGLSSPVTKSFNRGEC |
| 100 | 3E10- | Humanized 3E10 | MGWSCIILFLVATATGVHSDIVLTQSPASLAVSPGQRATITCR |
| | LC-h4 | Light Chain (with | ASKSVSTSSYSYMHWYQQKPGQPPKLLIKYASYLESGVPARF |
| | | signal peptide) | SGSGSGTDFTLTINPVEAEDAATYYCQHSREFPWTFGGGTKV |
| | | | EIKRTVAAPSVFIFPPSDEQLKSGTASVVCLLNNFYPREAKVQ |
| | | | WKVDNALQSGNSQESVTEQDSKDSTYSLSSTLTLSKADYEK |
| | | | HKVYACEVTHQGLSSPVTKSFNRGEC |
| 101 | 3E10- | Humanized 3E10 | MGWSCIILFLVATATGVHSDIVLTQSPASLAVSPGQRATITCR |
| | LC-h5 | Light Chain (with | ASKSVSTSSYSYMHWYQQKPGQPPKLLIKYASYLESGVPARF |
| | | signal peptide) | SGSGSGTDFTLNIHPVEAEDTANYYCQHSREFPWTFGGGTKV |
| | | | EIKRTVAAPSVFIFPPSDEQLKSGTASVVCLLNNFYPREAKVQ |
| | | | WKVDNALQSGNSQESVTEQDSKDSTYSLSSTLTLSKADYEK |
| | | | HKVYACEVTHQGLSSPVTKSFNRGEC |
| 102 | 3E10- | Humanized 3E10 | MGWSCIILFLVATATGVHSDIVLTQSPASLAVSPGQRATISCR |
| | LC-h6 | Light Chain (with | ASKSVSTSSYSYMHWYQQKPGQPPKLLIKYASYLESGVPARF |
| | | signal peptide) | SGSGSGTDFTLNIHPVEEEDTANYYCQHSREFPWTFGGGTKV |
| | | | EIKRTVAAPSVFIFPPSDEQLKSGTASVVCLLNNFYPREAKVQ |
| | | | WKVDNALQSGNSQESVTEQDSKDSTYSLSSTLTLSKADYEK |
| | | | HKVYACEVTHQGLSSPVTKSFNRGEC |
| 103 | NLS1 | Additionally | RASKSVSTSSYSYMHWYQQKPGQPPKLLIKY |
| | | contemplated | |
| | | sequences | |
| 104 | NLS2 | Additionally | RASKTVSTSSYSYMHWYQQKPGQPPKLLIKY |
| | | contemplated | |
| | | sequences | |
| 105 | NLS3 | Additionally | RVTITCRASKSVSTSSYSYMHWYQQKPGKAPKL |
| | | contemplated | |
| | | sequences | |
| 106 | NLS4 | Additionally | XRASKTVSTSSYSYMHWYQQKPGQPPKLLXKY (where (X) = |
| | (consensus) | contemplated | any residue, such as a basic residue (R or K) |
| | | sequences | |
| N/A | Linker | Additionally | GS |
| | 0 | contemplated | |
| | | sequences | |
| 107 | Linker | Additionally | GQSSRSS |
| | 1 | contemplated | |
| | | sequences | |
| 108 | Linker | Additionally | GQSSRSSSGGGSSGGGGS |
| | 2 | contemplated | |
| | | sequences | |
| 109 | Linker | Additionally | GSGS |
| | 3 | contemplated | |
| | | sequences | |
| 110 | Linker | Additionally | GGGS |
| | 4 | contemplated | |
| | | sequences | |
| 111 | Linker | Additionally | (Gly₄-Ser)₂ |
| | 5 | contemplated | |
| | | sequences | |
| 112 | Linker | Additionally | (Gly₄-Ser)₄ |
| | 6 | contemplated | |
| | | sequences | |
| 113 | Linker | Additionally | (Gly-Gly-Gly-Gly-Ser)₃ |
| | 7 | contemplated | |
| | | sequences | |
| 114 | Linker | Additionally | RADAAPGGGGSGGGGSGGGGS |
| | 8 | contemplated | |
| | | sequences | |
| 115 | Linker | Additionally | ASTKGPSVFPLAPLESSGS |
| | 9 | contemplated | |
| | | sequences | |
| 116 | CDRH1.3 | Additionally | GFTFSDYG |
| | | contemplated | |
| | | sequences | |
| 117 | CDRH1.4 | Additionally | GFTFSNYG |
| | | contemplated | |
| | | sequences | |
| 118 | CDRH2.2 | Additionally | ISSGSSTI |
| | | contemplated | |
| | | sequences | |
| 119 | CDRH2.2 | Additionally | ISSSSSTI |
| | | contemplated | |
| | | sequences | |
| 120 | CDRH3.2 | Additionally | ARRGLLLDY |
| | | contemplated | |
| | | sequences | |
| 121 | CDRL1.2 | Additionally | KSVSTSSYSY |
| | | contemplated | |
| | | sequences | |
| 122 | CDRL1.2 | Additionally | KTVSTSSYSY |
| | | contemplated | |
| | | sequences | |
| N/A | CDRL2.2 | Additionally | YAS |
| | | contemplated | |
| | | sequences | |
| 123 | CDRL3.2 | Additionally | QHSREFPWT |
| | | contemplated | |
| | | sequences | |
| 124 | | light chain variable | MTQSPSSLSASLGDRATITCRASKTVSTSSYSYMHWYQ |
| | | domain (VL) | QKPGQPPKLLIKYASYLESGVPSRFSGSGSGTDFTLTISS |
| | | | LQPEDAATYYCQHSREFPWTFGGGTKVEIK |
| 125 | | heavy chain variable | EVQLVESGGGLVQPGGSLRLSCAASGFTFSNYGMHWV |
| | | domain (VH) | RQAPGKGLEWVSYISSGSSTIYYADSVKGRFTISRDNAK |
| | | | NSLYLQMNSLRAEDTAVYYCARRGLLLDYWGQGTTV |
| | | | TVSS |

1. A conjugate of Formula (I): 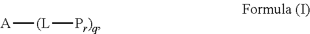
wherein in Formula (I):
A is an antibody or antigen-binding fragment thereof comprising a heavy chain variable region (VH) CDR1 comprising the amino acid sequence of SEQ ID NO:58, CDR2 comprising the amino acid sequence of SEQ ID NO:59, CDR3 comprising SEQ ID NO:60; and a light chain variable region (VL) CDR1 comprising the amino acid sequence of SEQ ID NO:61, CDR2 comprising the amino acid sequence of SEQ ID NO:62, CDR3 comprising the amino acid sequence of SEQ ID NO:63;
L is a linker;
P is a payload moiety;
r is an integer from 1 to 4; and
q is an integer from 1 to 16.

2. The conjugate of claim 1, wherein the linker L comprises one or more groups selected from optionally substituted alkylene, optionally substituted alkenylene, optionally substituted alkynylene, optionally substituted arylene, optionally substituted cycloalkylene, optionally substituted heteroalkylene, optionally substituted heteroarylene, optionally substituted heterocycloalkylene, —NR^{a}—, —N═CR^{a}—, —CR^{a}═N—, —S—, —S(O)—, —S(O)₂—, —OP(O)OR^{a}—, —OP(O)OR^{a}O—, —P(O)OR^{a}O—, —O—, —CR^{b}₂—, —[CR^{b}₂)₁₋₁₂O]₁₋₅₀—, —C(O)—, —C(S)—, —C(═N—OH)—, —C(NR^{a})—, —C(NH₂Cl)—, —C(O)O—, —OC(O)—, —C(O)S—, —SC(O)—, —C(O)NR^{a}—, —NR^{a}C(O)—, —C(O)NR^{a}SO₂—, —SO₂NRC(O)—, —OC(O)O—, —OC(O)S—, —SC(O)O—, —OC(O)NR^{a}—, —NR^{a}C(O)O—, —SC(O)NR^{a}—, —NR^{a}C(O)S—, —S(O)ₜN(R^{a})— (where t is 1 or 2), —N(R^{a})S(O)ₜ— (where t is 1 or 2), and —X_{AA}—;
each R^{a} is independently selected at each occurrence from hydrogen, optionally substituted alkyl, optionally substituted fluoroalkyl, optionally substituted cycloalkyl, optionally substituted cycloalkylalkyl, optionally substituted alkenyl, optionally substituted fluoroalkenyl, optionally substituted cycloalkenyl, optionally substituted cycloalkenylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted alkenyl, optionally substituted fluoroalkenyl, optionally substituted cycloalkenyl, optionally substituted cycloalkenylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
two independent R^{b} groups or an R^{a} and an R^{b} can be joined together to form an optionally substituted cycle; and
—X_{AA}— is an amino acid sequence comprising 1 to 6 amino acid moieties.

3. The conjugate of claim 2, wherein each amino acid moiety of —X_{AA}— is independently selected from alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), valine (Val), citrulline (Cit), and homocitrulline (HoCit).

4. The conjugate of any one of claims 1 to 3, wherein the linker L comprises one or more groups selected from optionally substituted C₁-C₁₈ alkylene, —C≡C—, —CR^{a}═CR^{a}—, optionally substituted 6- to 14-membered arylene, optionally substituted C₃-C₂₀ cycloalkylene, —[CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —N═CR^{a}—, —CR^{a}═N—, —S—, —OP(O)OR^{a}O—, —O—, —CR^{b}₂—, —C(O)—, —C(O)O—, —OC(O)—, —C(O)S—, —SC(O)—, —C(O)NR^{a}—, —NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, and —X_{AA}—.

5. The conjugate of any one of claims 1 to 4, wherein the linker L comprises one or more groups selected from optionally substituted C₁-C₁₆ alkylene, —C≡C—, —CR^{a}—CR^{a}—, optionally substituted phenylene, optionally substituted C₃-C₆ cycloalkylene, —[CH₂CH₂O]₁₋₁₆—, —[CH₂CH₂CH₂O]₁₋₁₆—, optionally substituted 5- to 6-membered heteroarylene, optionally substituted 5- to 20-membered heterocycloalkylene, —NR^{a}—, —N═CR^{a}—, —CR^{a}═N—, —S—, —OP(O)OR^{a}O—, —O—, —CR^{b}₂—, —C(O)—, —C(O)O—, —OC(O)—, —C(O)NR^{a}—, —NR^{a}C(O)—, —OC(O)O—, —OC(O)NR^{a}—, —NR^{a}C(O)O—, and —X_{AA}—.

6. The conjugate of any one of claims 1 to 5, wherein the linker L comprises one or more groups selected from —[C(R^{b})₂]₁₋₁₆—, —C≡C—, —CR^{a}═CR^{a}—, —[CH₂CH₂O]₁₋₁₆—, —NR^{a}—, —N═CR^{a}—, —CR^{a}═N—, —S—, —OP(O)OR^{a}O—, —O—, —C(O)—, —C(O)O—, —OC(O)—, —C(O)NR^{a}—, —NR^{a}C(O)—, —OC(O)O—, —OC(O)NR^{a}—, —NR^{a}C(O)O—, —X_{AA}—, 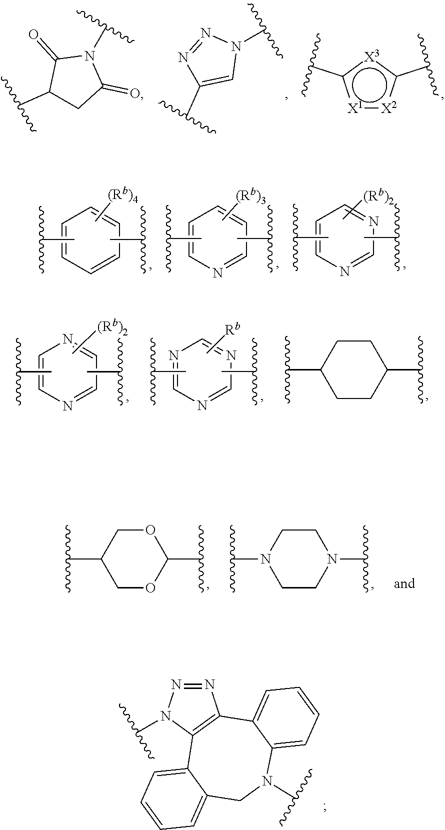
and
X¹, X², and X³ are independently selected at each occurrence from NR^{a}, N, CR^{b}, S, and 0.

7. The conjugate of any one of claims 1 to 6, wherein the linker L is of Formula (L-1): 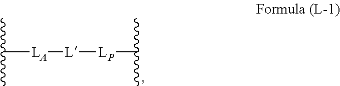
wherein in Formula (L-1):
L^{A} is a connecting moiety through which A is covalently attached to L′;
L′ is a bond or comprises one or more groups selected from optionally substituted alkylene, optionally substituted alkenylene, optionally substituted alkynylene, optionally substituted arylene, optionally substituted cycloalkylene, optionally substituted heteroalkylene, optionally substituted heteroarylene, optionally substituted heterocycloalkylene, —NR^{a}—, —N═CR^{a}—, —CR^{a}═N—, —S—, —S(O)—, —S(O)₂—, —OP(O)OR^{a}O—, —O—, —CR^{b}₂—, —[(CR^{b}₂)₁₋₁₂O]₁₋₅₀—, —C(O)—, —C(S)—, —C(NR^{a})—, —C(NH₂Cl)—, —C(O)O—, —OC(O)—, —C(O)S—, —SC(O)—, —C(O)NR^{a}—, —NR^{a}C(O)—, —C(O)NR^{a}SO₂—, —SO₂NRC(O)—, —OC(O)O—, —OC(O)S—, —SC(O)O—, —OC(O)NR^{a}—, —NR^{a}C(O)O—, —SC(O)NR^{a}—, —NR^{a}C(O)S—, —S(O)ₜN(R^{a})—, —N(R^{a})S(O)ₜ—, and —X_{AA}—; and
L_{P} is a connecting moiety through which P is covalently attached to L′.

8. The conjugate of any one of claims 1 to 7, wherein the linker L comprises at least one cleavable moiety.

9. The conjugate of claim 8, wherein the cleavable moiety comprises an acid-labile moiety, a reducibly-labile moiety, or an enzymatically-labile moiety.

10. The conjugate of claim 8 or claim 9, wherein the cleavable moiety comprises one or more groups selected from: 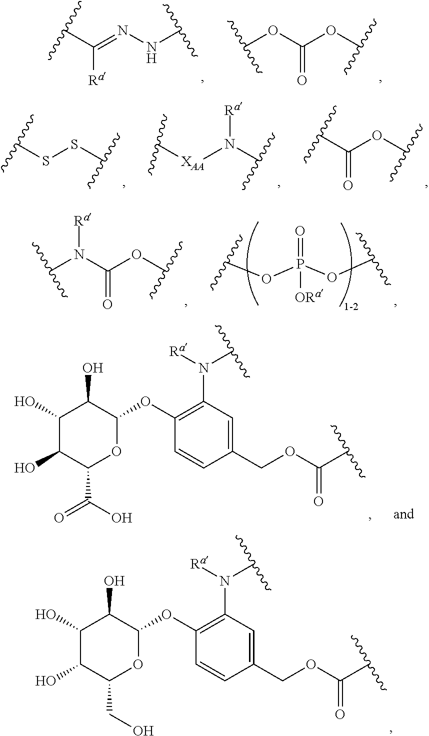
wherein:
each R^{a′} is independently selected at each occurrence from hydrogen, optionally substituted alkyl, and optionally substituted heteroalkyl.

11. The conjugate of any one of claims 9 to 10, wherein the cleavable moiety comprises the reducibly-labile moiety —S—S—.

12. The conjugate of any one of claims 1 to 8, wherein the linker L is of Formula (L-10): 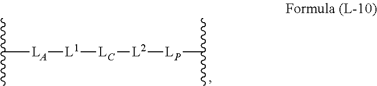
wherein in Formula (L-10):
L_{A} is selected from a bond, —NR^{a′}—, and —S—;
L¹ is a bond or comprises one or more groups selected from optionally substituted C₁-C₁₈ alkylene, —C≡C—, —CR^{a}═CR^{a}—, optionally substituted 6- to 14-membered arylene, optionally substituted C₃-C₂₀ cycloalkylene, —[CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —N═CR^{a}—, —CR^{a}═N—, —S—, —OP(O)OR^{a}O—, —O—, —CR^{b}₂—, —C(O)—, —C(O)O—, —OC(O)—, —C(O)S—, —SC(O)—, —C(O)NR^{a}—, —NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, and —X_{AA}—;
L_{C} is selected from an acid-labile moiety, a reducibly-labile moiety, and an enzymatically-labile moiety;
L² is a bond or comprises one or more groups selected from optionally substituted C₁-C₁₈ alkylene, —C≡C—, —CR^{a}═CR^{a}—, optionally substituted 6- to 14-membered arylene, optionally substituted C₃-C₂₀ cycloalkylene, —[CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —N═CR^{a}—, —CR^{a}═N—, —S—, —OP(O)OR^{a}O—, —O—, —CR^{b}₂—, —C(O)—, —C(O)O—, —OC(O)—, —C(O)S—, —SC(O)—, —C(O)NR^{a}—, —NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, and —X_{AA}—;
L_{P} is selected from a bond, —NR^{a′}—, —S—, and —O—;
each R^{a} is independently selected at each occurrence from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
each R^{a′} is independently selected at each occurrence from hydrogen, optionally substituted alkyl, and optionally substituted heteroalkyl;
each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or two independent R^{b} groups are taken together to form optionally substituted cycloalkyl, optionally substituted aryl, optionally substituted heterocycloalkyl, or optionally substituted heteroaryl; and
X_{AA}— is an amino acid sequence comprising 1 to 4 amino acid moieties.

13. The conjugate of claim 11, wherein L_{C} is selected from: 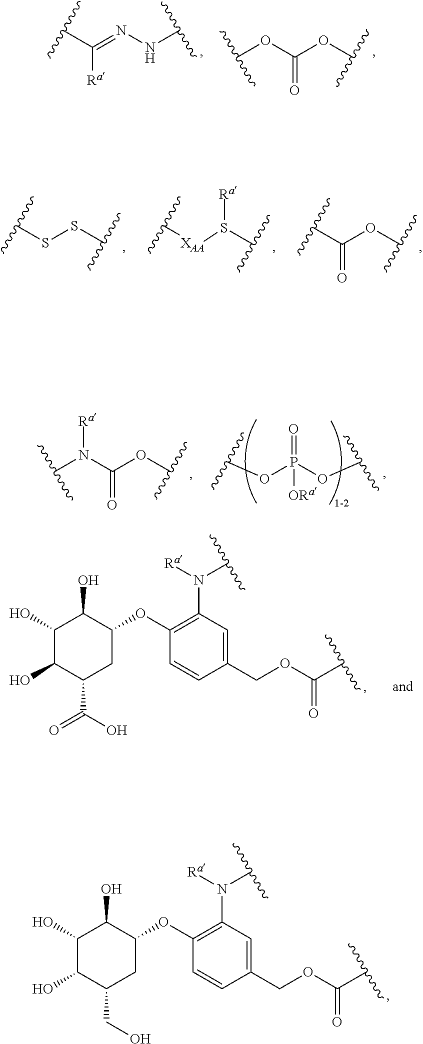
wherein:
each R^{a′} is independently selected at each occurrence from hydrogen, optionally substituted alkyl, and optionally substituted heteroalkyl.

14. The conjugate of claim 12 or claim 13, wherein L_{C} is —S—S—.

15. The conjugate of any one of claims 1 to 7, wherein the linker L is of Formula (L-11): 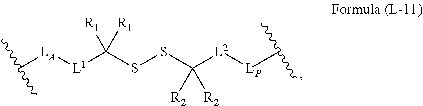
wherein in Formula (L-11):
L_{A} is selected from a bond, —NH—, and —S—;
L¹ is a bond or comprises one or more groups selected from —[C(R^{b})₂]₁₋₁₆—, —C≡C—, —CR^{a}═CR^{a}—, —[CH₂CH₂O]₁₋₁₆—, —NR^{a}—, —N═CR^{a}—, —CR^{a}═N—, —S—, —OP(O)OR^{a}O—, —O—, —C(O)—, —C(O)O—, —OC(O)—, —C(O)NR^{a}—, —NR^{a}C(O)—, —OC(O)O—, —X_{AA}—, —OC(O)NR^{a}—, —NR^{a}C(O)O—, 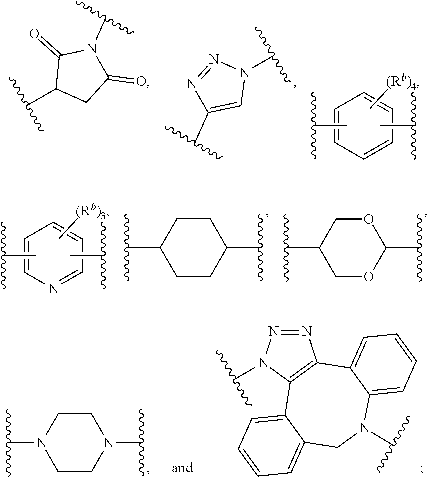
L² is a bond or comprises one or more groups selected from —[C(Rh)₂]₁₋₁₆-, —C≡C—, —CR^{a}═CR^{a}—, —[CH₂CH₂O]₁₋₁₆—, —NR^{a}—, —N═CR^{a}—, —CR^{a}═N—, —S—, —OP(O)OR^{a}O—, —O—, —C(O)—, —C(O)O—, —OC(O)—, —C(O)NR^{a}—, —NR^{a}C(O)—, —OC(O)O—, —X_{AA}—, —OC(O)NR^{a}—, —NR^{a}C(O)O—, 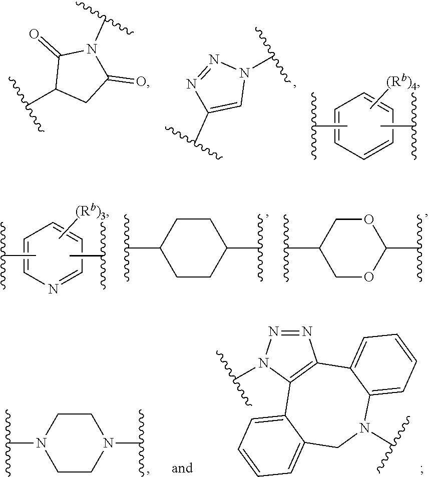
L_{P} is selected from a bond, —NR^{a′}—, and —O—;
each R₁ is independently selected from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or both R₁ groups are taken together to form optionally substituted cycloalkyl;
each R₂ is independently selected from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or both R₂ groups are taken together to form optionally substituted cycloalkyl;
each R^{a} is independently selected at each occurrence from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted alkyl;
each R^{b} is independently selected at each occurrence from hydrogen, halide, —OH, —SO₃H, —OPO₃H₂, —PO₃H₂, —CO₂R, —NR^{a}₂, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl; and
—X_{AA}— is an amino acid sequence comprising 2 to 4 amino acid moieties.

16. The conjugate of any one of claims 1 to 7, wherein the linker L is of Formula (L-12): 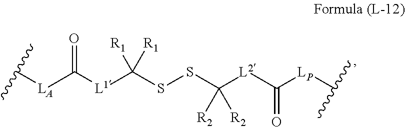
wherein in Formula (L-12):
L_{A} is selected from a bond and —NH—;
L^{1′} comprises one or more groups selected from —[C(R^{b})₂]₁₋₁₀—, —[CH₂CH₂O]₁₋₁₀—, —NR^{a}—, —O—, —C(O)—, —C(O)O—, —OC(O)—, —C(O)NR^{a}—, —NR^{a}C(O)—, —OC(O)O—, —X_{AA}—, —OC(O)NR^{a}—, —NR^{a}C(O)O—, 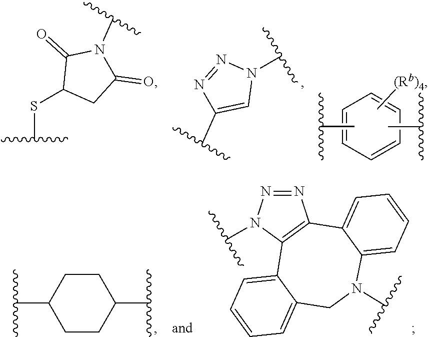
L^{2′} comprises one or more groups selected from —[C(R^{b})₂]o, —[CH₂CH₂]₁₋₁₀-, —NR^{a}—, —O—, —C((O)O—, —OC(O)—, —C(O)NR^{a}—, —NR^{a}C(O)—, —OC(O)O—, —X_{AA}—, —OC(O)NR^{a}—, —NR^{a}C(O)O—, 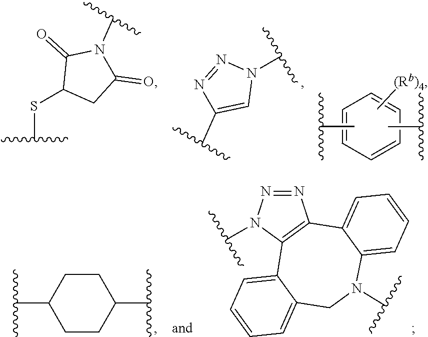
L_{P} is selected from a bond and —NR^{a′};
each R₁ is independently selected from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or both R₁ groups are taken together to form optionally substituted C₃-C₆ cycloalkyl;
each R₂ is independently selected from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or both R₂ groups are taken together to form optionally substituted C₃-C₆ cycloalkyl;
each R^{a} is independently selected at each occurrence from hydrogen, optionally substituted C₁-C₆ alkyl, optionally substituted C₁-C₆ fluoroalkyl, and optionally substituted C₃-C₆ cycloalkyl;
each R^{a′} is independently selected at each occurrence from hydrogen and optionally substituted C₁-C₆ alkyl;
each R^{b} is independently selected at each occurrence from hydrogen, halide, —OH, —SO₃H, —OPO₃H₂, —PO₃H₂, —CO₂R, —NR^{a}₂, optionally substituted C₁-C₆ alkyl, optionally substituted C₁-C₆ fluoroalkyl, and optionally substituted C₃-C₆ cycloalkyl; or two independent R^{b} groups attached to the same carbon atom are taken together to form optionally substituted C₃-C₆ cycloalkyl; and
—X_{AA}— is an amino acid sequence comprising 2 or 3 amino acid moieties.

17. The conjugate of claim 15 or claim 16, wherein at least one R₁ or R₂ is other than hydrogen.

18. The conjugate of any one of claims 15 to 17, wherein at least one R₁ is an optionally substituted C₁-C₈ alkyl.

19. The conjugate of any one of claims 15 to 17, wherein each R₁ is independently an optionally substituted C₁-C₈ alkyl.

20. The conjugate of any one of claims 15 to 19, wherein at least one R₂ is an optionally substituted C₁-C₈ alkyl.

21. The conjugate of any one of claims 15 to 19, wherein each R₂ is independently an optionally substituted C₁-C₈ alkyl.

22. The conjugate of any one of claims 1 to 10, wherein the linker L is selected from:

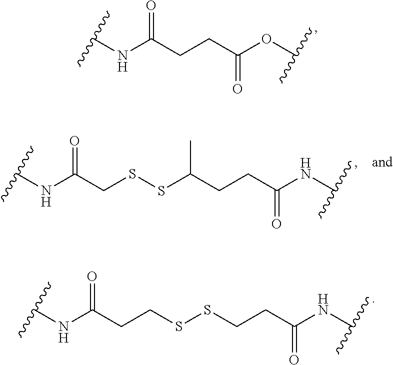 [SMILES: *NC(=O)CCC(=O)O* ; *NC(=O)CCC(C)SSCC(=O)N* ; *NC(=O)CCSSCCC(=O)N*]

23. The conjugate of any one of claims 1 to 10, wherein the linker L is:

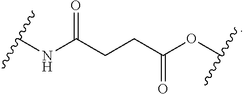 [SMILES: *NC(=O)CCC(=O)O*]

24. The conjugate of any one of claims 1 to 10, wherein the linker L is:

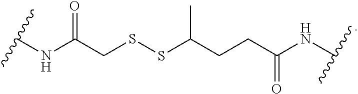 [SMILES: *NC(=O)CCC(C)SSCC(=O)N*]

25. The conjugate of any one of claims 1 to 10, wherein the linker L is:

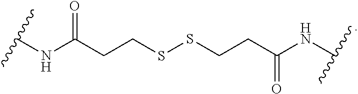 [SMILES: *NC(=O)CCSSCCC(=O)N*]

26. The conjugate of any one of claims 1 to 16 wherein the linker L is selected from:

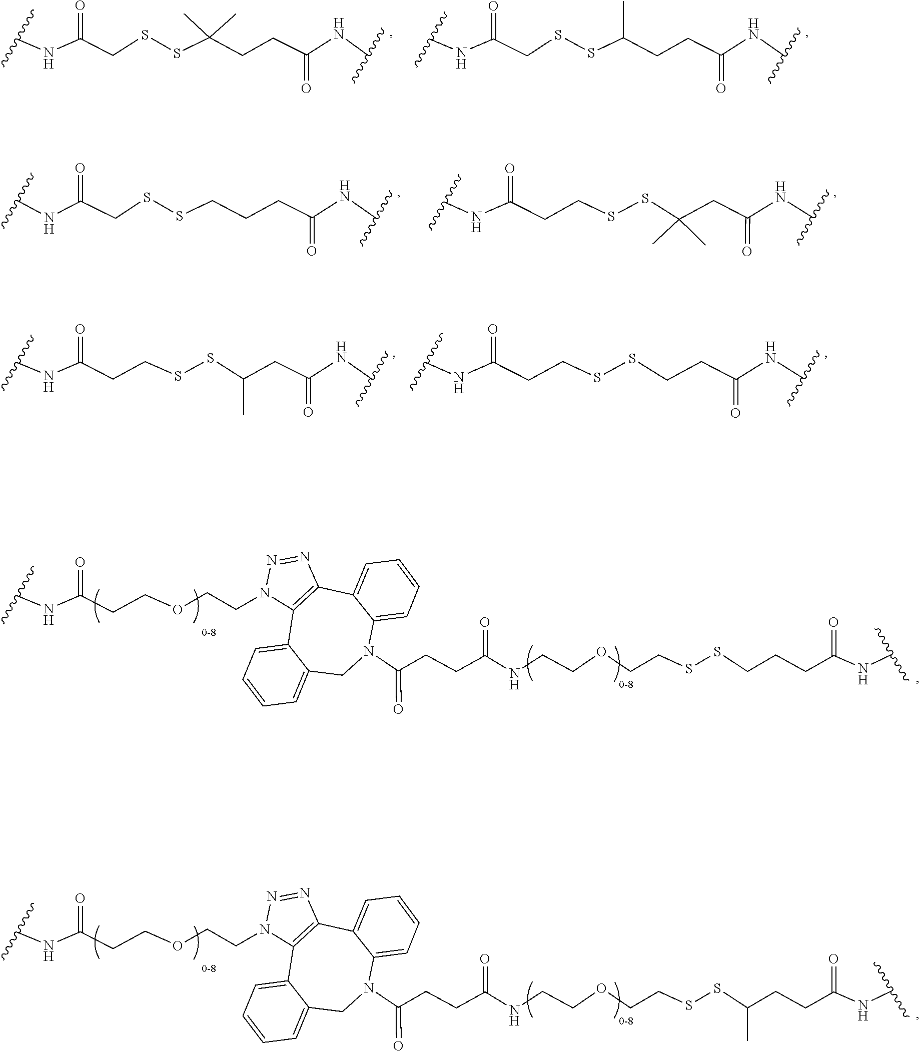

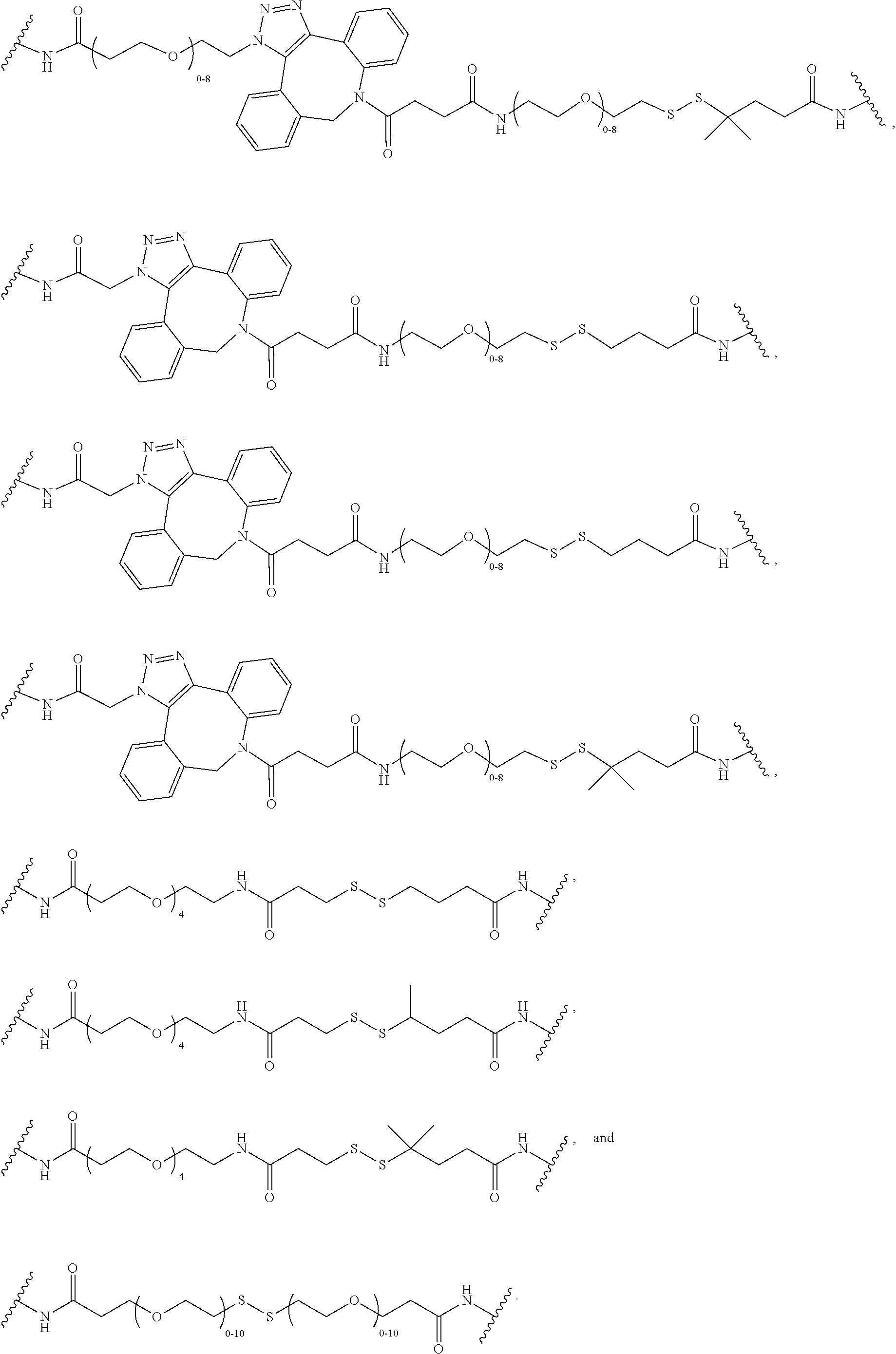

27. The conjugate of any one of claims 8 to 26, wherein the linker is a cleavable linker.

28. The conjugate of claim 27, wherein the cleavable linker is conjugated to a lysine of the 3E10 antibody or antigen-binding fragment thereof thereof.

29. The conjugate of claim 27, wherein the cleavable linker is conjugated to a cysteine of the antibody or antigen-binding fragment thereof.

30. The conjugate of claim 27, wherein the cleavable linker is conjugated to a histidine of the antibody or antigen-binding fragment thereof.

31. The conjugate of claim 27, wherein the cleavable linker is conjugated to an arginine of the antibody or antigen-binding fragment thereof.

32. The conjugate of claim 27, wherein the cleavable linker is conjugated to an aspartic acid of the antibody or antigen-binding fragment thereof.

33. The conjugate of claim 27, wherein the cleavable linker is conjugated to a glutamine of the antibody or antigen-binding fragment thereof.

34. The conjugate of claim 33, wherein the conjugate has a drug to antibody ratio (DAR) of at least 4:1.

35. The conjugate of claim 33 or 34, wherein the linker is a branched linker attached to at least two copies of the oligonucleotide moiety.

36. The conjugate of claim 27, wherein the cleavable linker is conjugated to a serine of the antibody or antigen-binding fragment thereof.

37. The conjugate of claim 27, wherein the cleavable linker is conjugated to a threonine of the antibody or antigen-binding fragment thereof.

38. The conjugate of claim 27, wherein the cleavable linker is conjugated to a tyrosine of the antibody or antigen-binding fragment thereof.

39. The conjugate of any one of claims 1 to 7, wherein the linker L is of Formula (L-20): 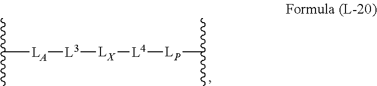
wherein in Formula (L-20):
L_{A} is selected from a bond, —NR^{a′}—, and —S—;
L³ is a bond or comprises one or more groups selected from —[C(R^{b})₂]₁₋₈—, —NR^{a}—, —C(O)—, —C(S)—, —C(NR^{a})—, —C(NH₂Cl)—, —C≡C—, —CR^{a}═CR^{a}—, optionally substituted 6- to 14-membered arylene, optionally substituted C₃-C₂₀ cycloalkylene, optionally substituted 5- to 18-membered heteroarylene, and optionally substituted 3- to 20-membered heterocycloalkylene;
L_{X} comprises one or more groups selected from optionally substituted C₁-C₁s alkylene, —C≡C—, —CR^{a}—CR^{a}—, optionally substituted 6- to 14-membered arylene, optionally substituted C₃-C₂₀ cycloalkylene, —[CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —S—, —O—, —CR^{b}₂—, —C(O)—, —C(S)—, —C(NR^{a})—, —C(NH₂Cl)—, —C(O)O—, —OC(O)—, —C(O)S—, —SC(O)—, —C(O)NR^{a}—, and —NR^{a}C(O)—;
L⁴ is a bond or comprises one or more groups selected from —[C(R^{b})₂]₁₋₈—, —N^{a}—, —C(O)—, —C(S)—, —C(NR^{a})—, —C(NH₂Cl)—, —C≡C—, —CR^{a}═CR^{a}—, optionally substituted 6- to 14-membered arylene, optionally substituted C₃-C₂₀ cycloalkylene, optionally substituted 5- to 18-membered heteroarylene, and optionally substituted 3- to 20-membered heterocycloalkylene;
L_{P} is selected from a bond, —NR^{a′}—, —S—, and —O—;
each R^{a} is independently selected at each occurrence from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
each R^{a′} is independently selected at each occurrence from hydrogen, optionally substituted alkyl, and optionally substituted heteroalkyl; and
each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or two independent R^{b} groups are taken together to form optionally substituted cycloalkyl, optionally substituted aryl, optionally substituted heterocycloalkyl, or optionally substituted heteroaryl.

40. The conjugate of any one of claims 1 to 7, wherein the linker L is of Formula (L-21): 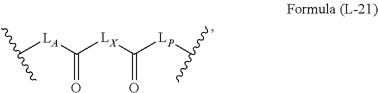
wherein in Formula (L-21):
L_{A} is selected from a bond and —NH—;
L_{X} comprises one or more groups selected from optionally substituted —[C(R^{b})₂]₁₋₁₆—, —C≡C—, —CR^{a}═R^{a}—, —[CH₂CH₂CH₂O]₁₋₁₆—, —NR^{a}—, —O—, —C(O)—, —C(O)O—, —OC(O)—, —C(O)S—, —SC(O)—, —C(O)NR^{a}—, —NR^{a}C(O)—, 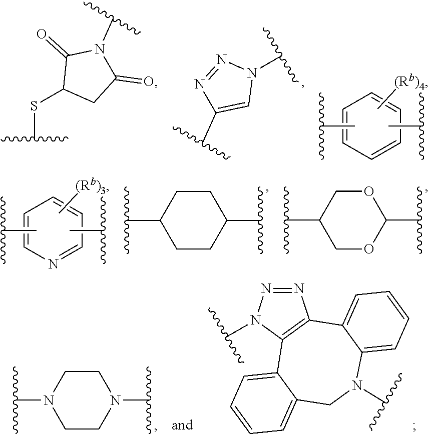
L_{P} is selected from a bond and —NR^{a′};
each R^{a} is independently selected at each occurrence from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted C₁-C₆ alkyl; and
each R^{b} is independently selected at each occurrence from hydrogen, halide, —OH, —SO₃H, —OPO₃H₂, —PO₃H₂, —CO₂R, —NR^{a}₂, optionally substituted C₁-C₅ alkyl, optionally substituted C₁-C₅ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl.

41. The conjugate of any one of claims 1 to 7, wherein the linker L is of Formula (L-22a) or Formula (L-22b): 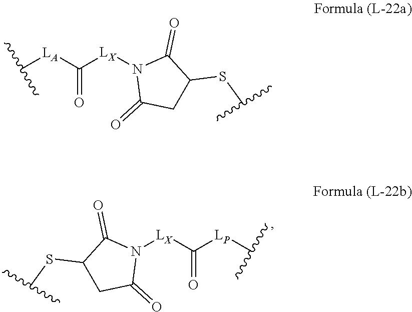
wherein in Formulas (L-22a) and (L-22b):
L_{A} is selected from a bond and —NH—;
L_{X} comprises one or more groups selected from optionally substituted —[C(R^{b})₂]₁₋₁₀—, —C≡C—, —CR^{a}═R^{a}—, —[CH₂CH₂CH₂O]₁₋₁₀—, —NR^{a}—, —C(O)—, —C(O)O—, —OC(O)—, —C(O)S—, —SC(O)—, —C(O)NR^{a}—, —NR^{a}C(O)—, 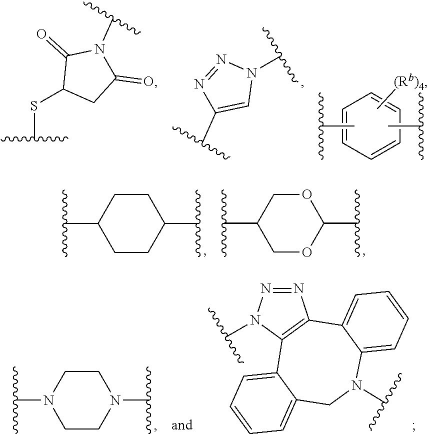
L_{P} is selected from a bond and —NR^{a′};
each R^{a} is independently selected at each occurrence from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted C₁-C₆ alkyl; and
each R^{b} is independently selected at each occurrence from hydrogen, halide, —OH, —SO₃H, —OPO₃H₂, —PO₃H₂, —CO₂R, —NR^{a}₂, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl.

42. The conjugate of claim 1, wherein the linker L is selected from:

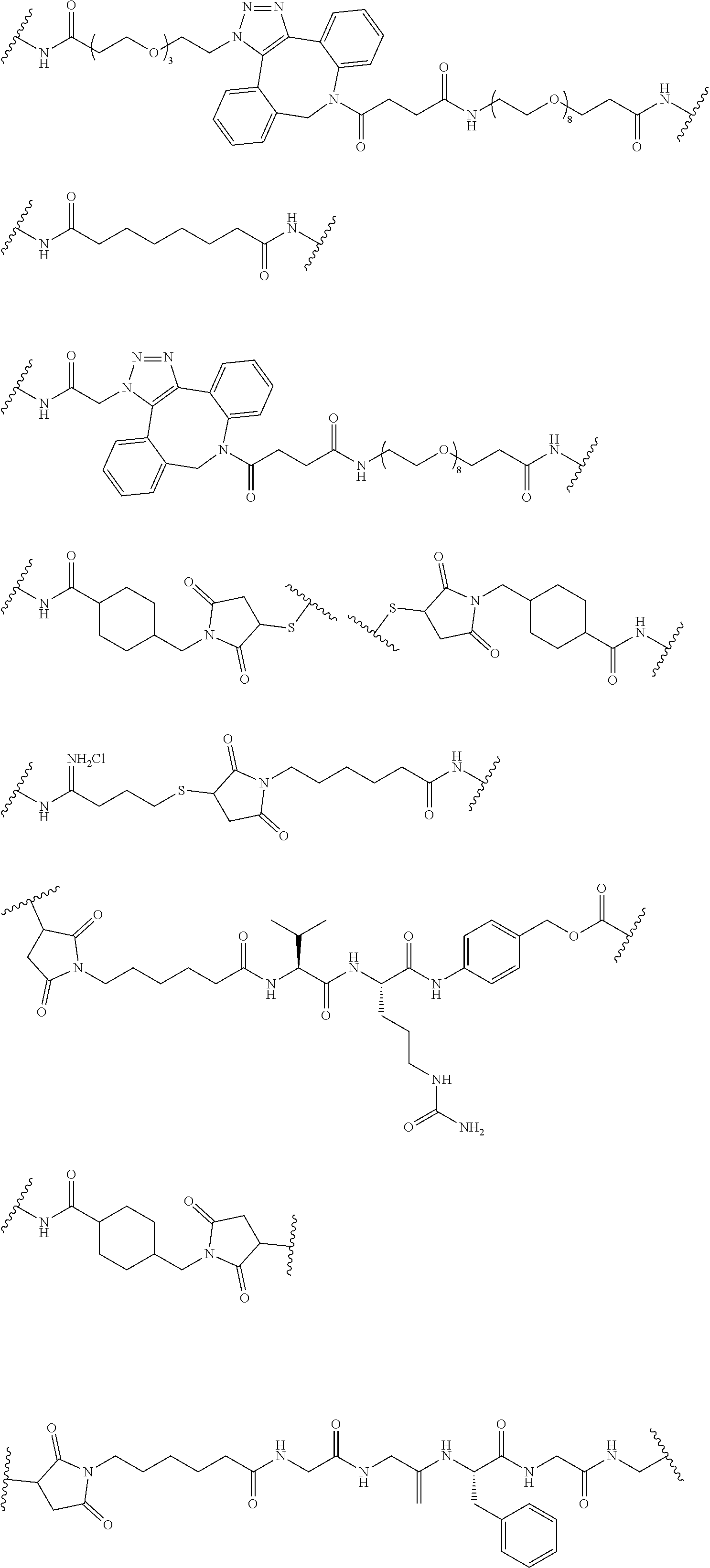

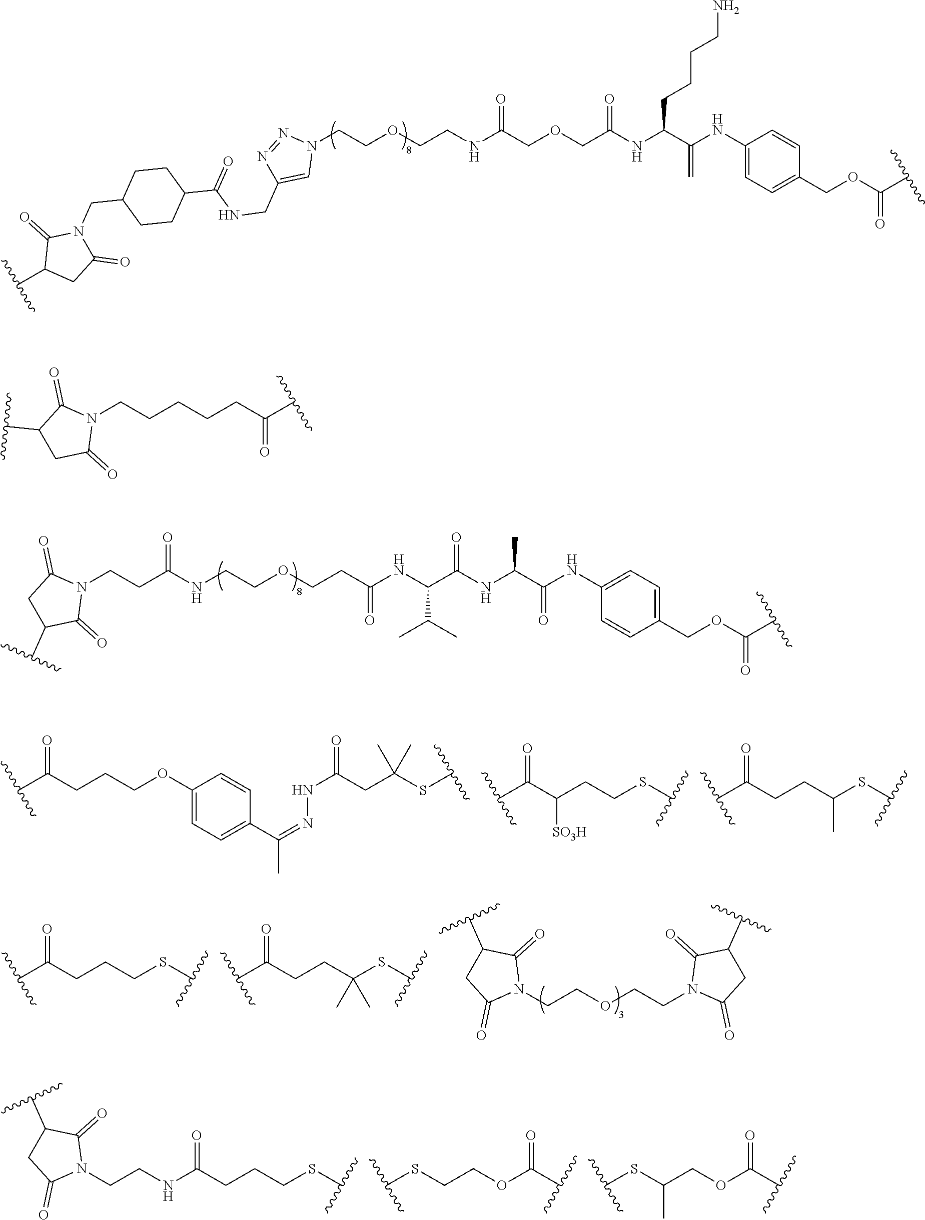

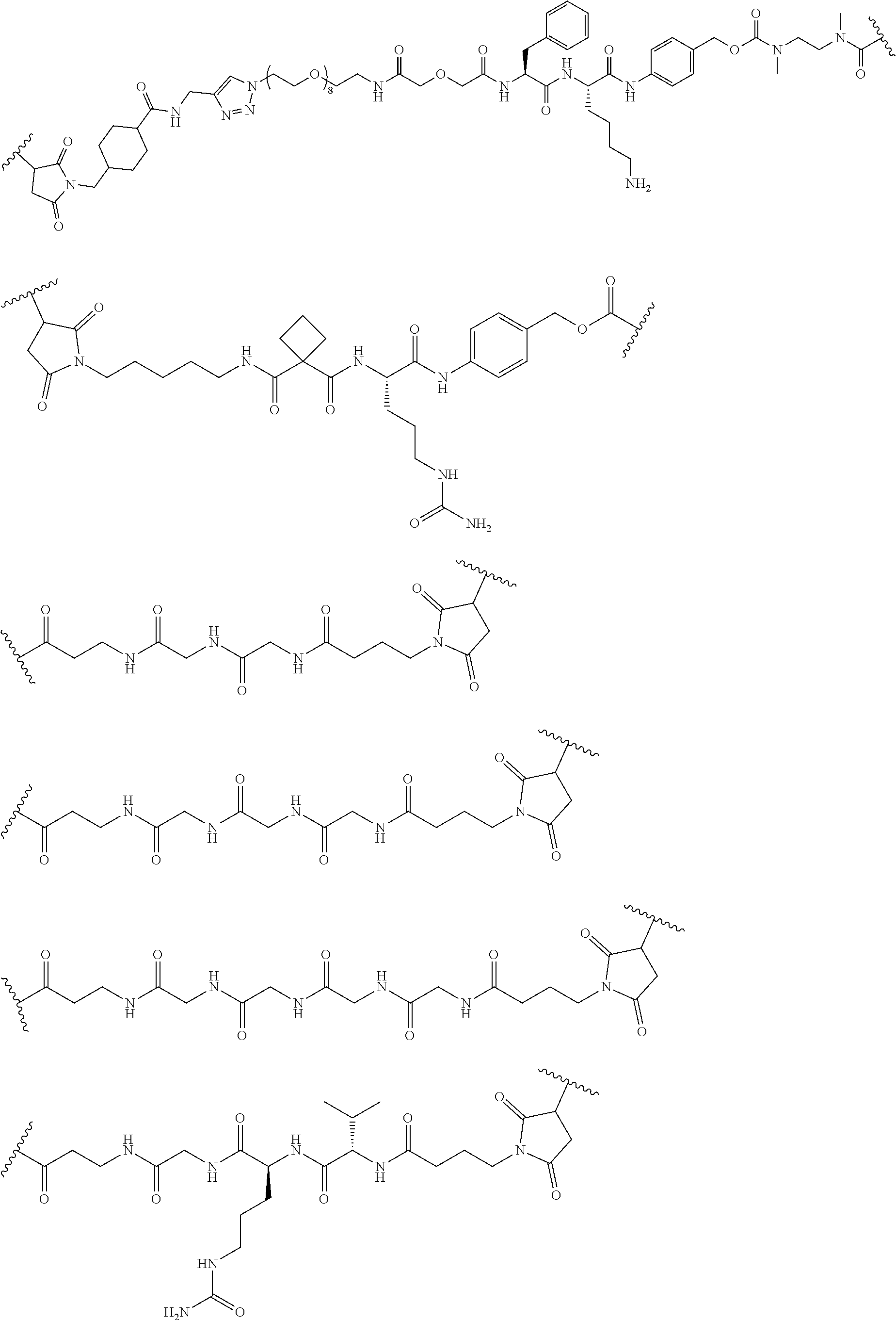

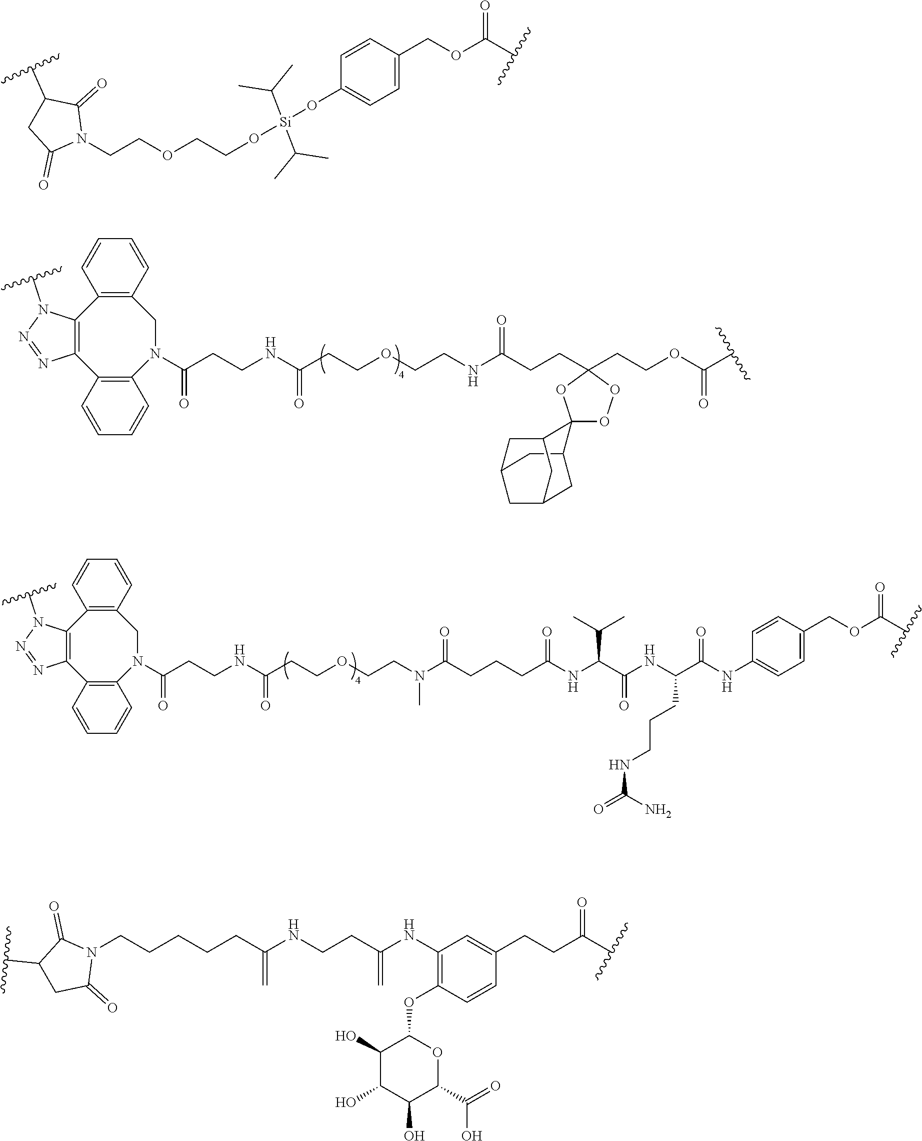

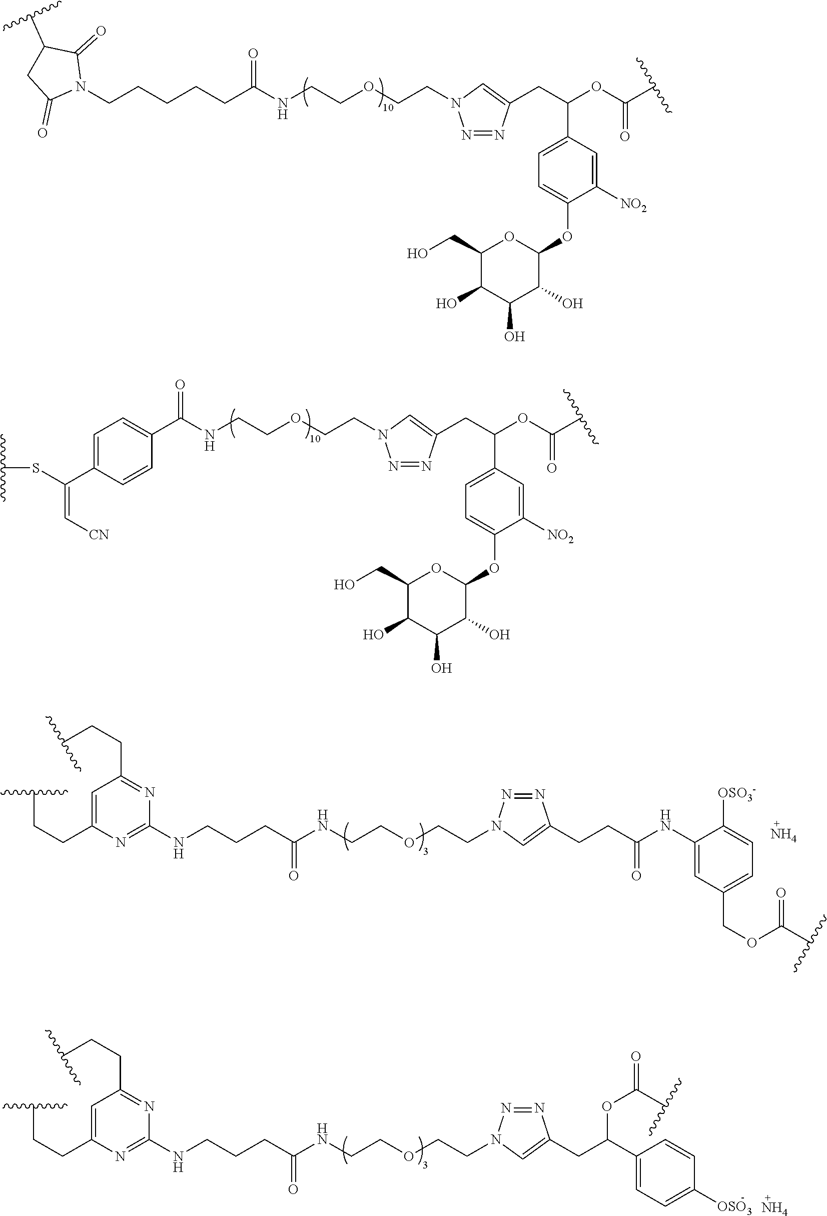

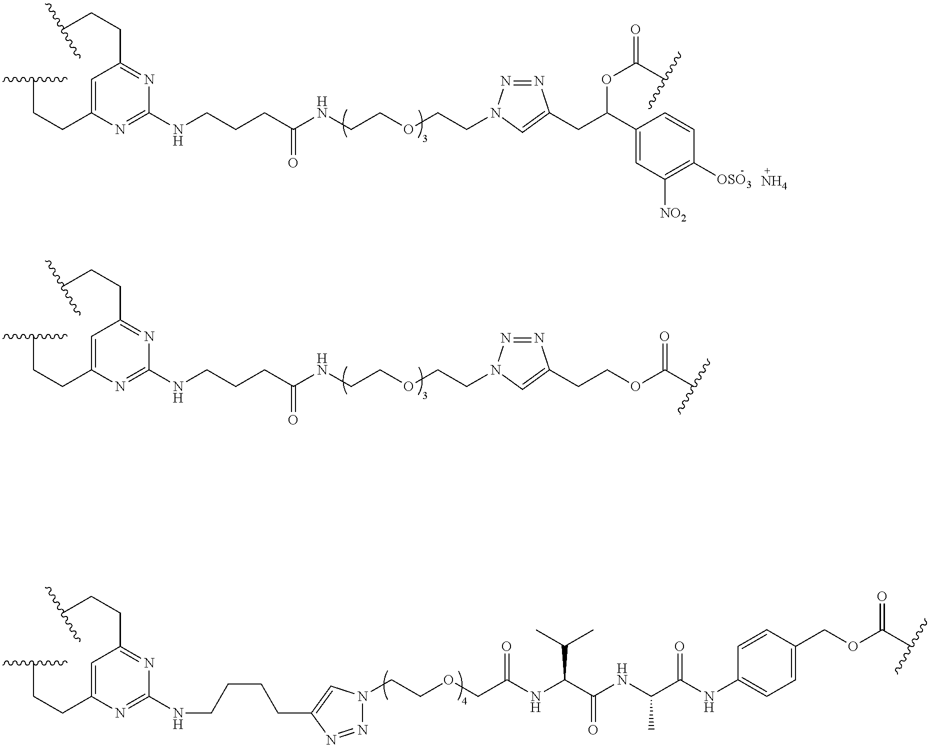

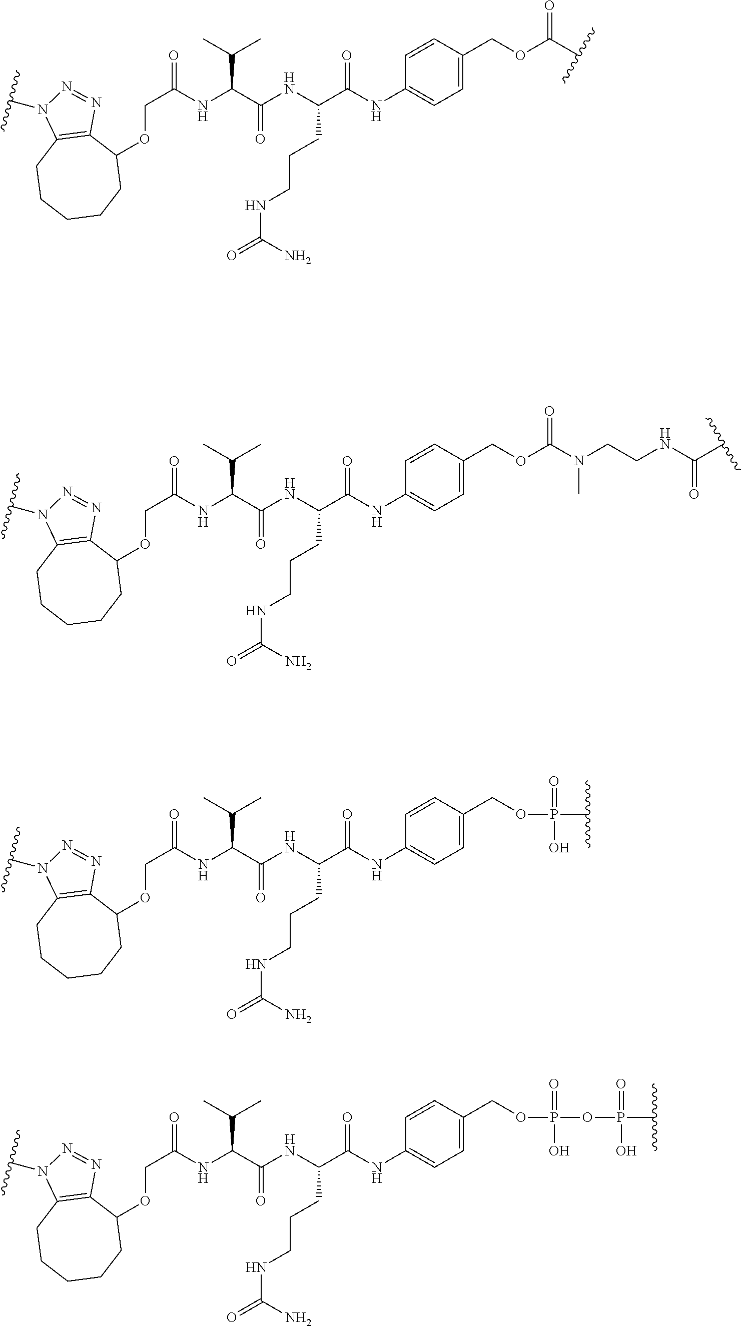 [SMILES: *C(=O)OCc1ccc(NC(=O)[C@H](CCCNC(N)=O)NC(=O)[C@@H](NC(=O)COC2CCCCCc3c2nnn3*)C(C)C)cc1 ; *C(=O)NCCN(C)C(=O)OCc1ccc(NC(=O)[C@H](CCCNC(N)=O)NC(=O)[C@@H](NC(=O)COC2CCCCCc3c2nnn3*)C(C)C)cc1 ; *n1nnc2c1CCCCCC2OCC(=O)N[C@H](C(=O)N[C@@H](CCCNC(N)=O)C(=O)Nc1ccc(COP(*)(=O)O)cc1)C(C)C ; *n1nnc2c1CCCCCC2OCC(=O)N[C@H](C(=O)N[C@@H](CCCNC(N)=O)C(=O)Nc1ccc(COP(=O)(O)OP(*)(=O)O)cc1)C(C)C]

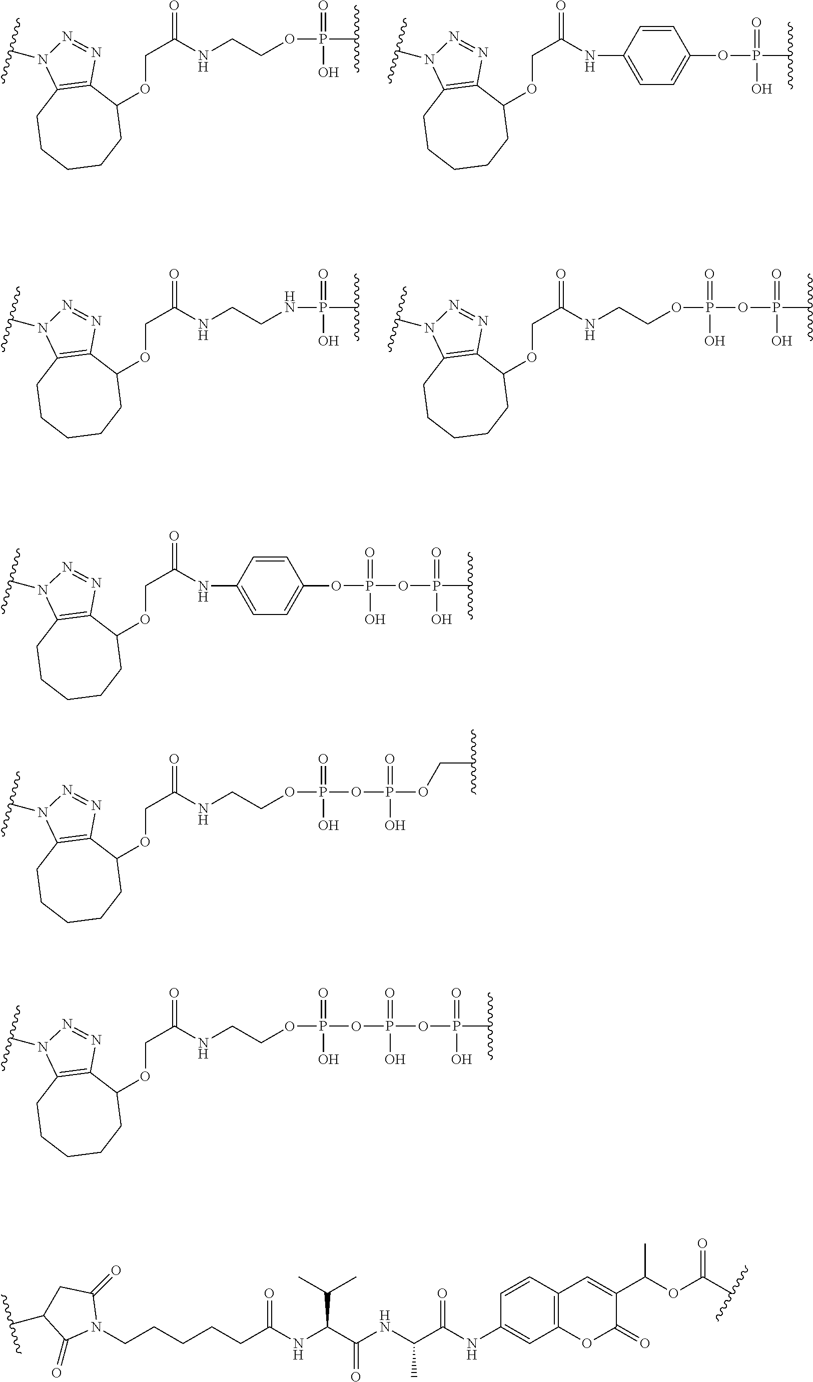

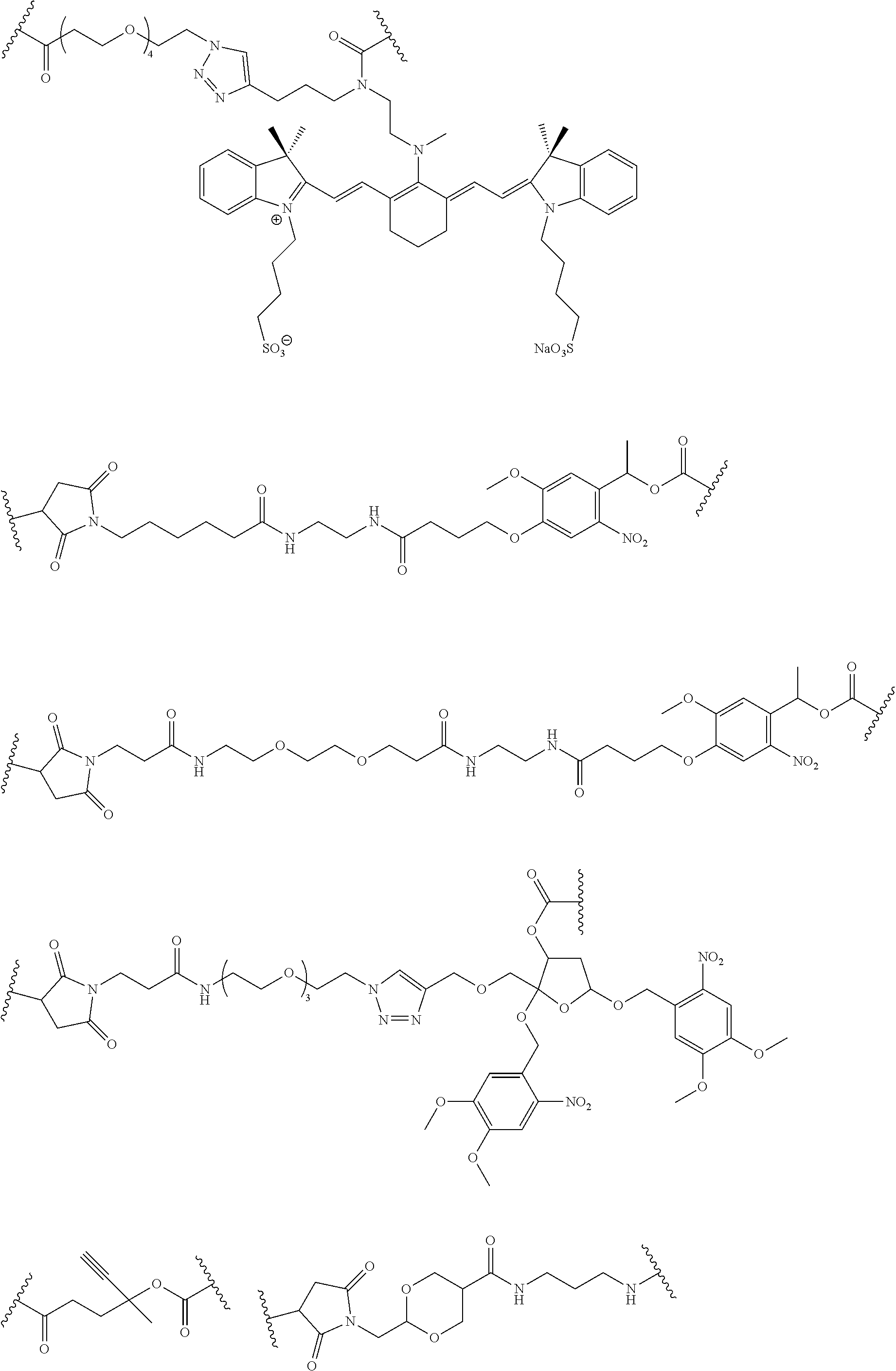

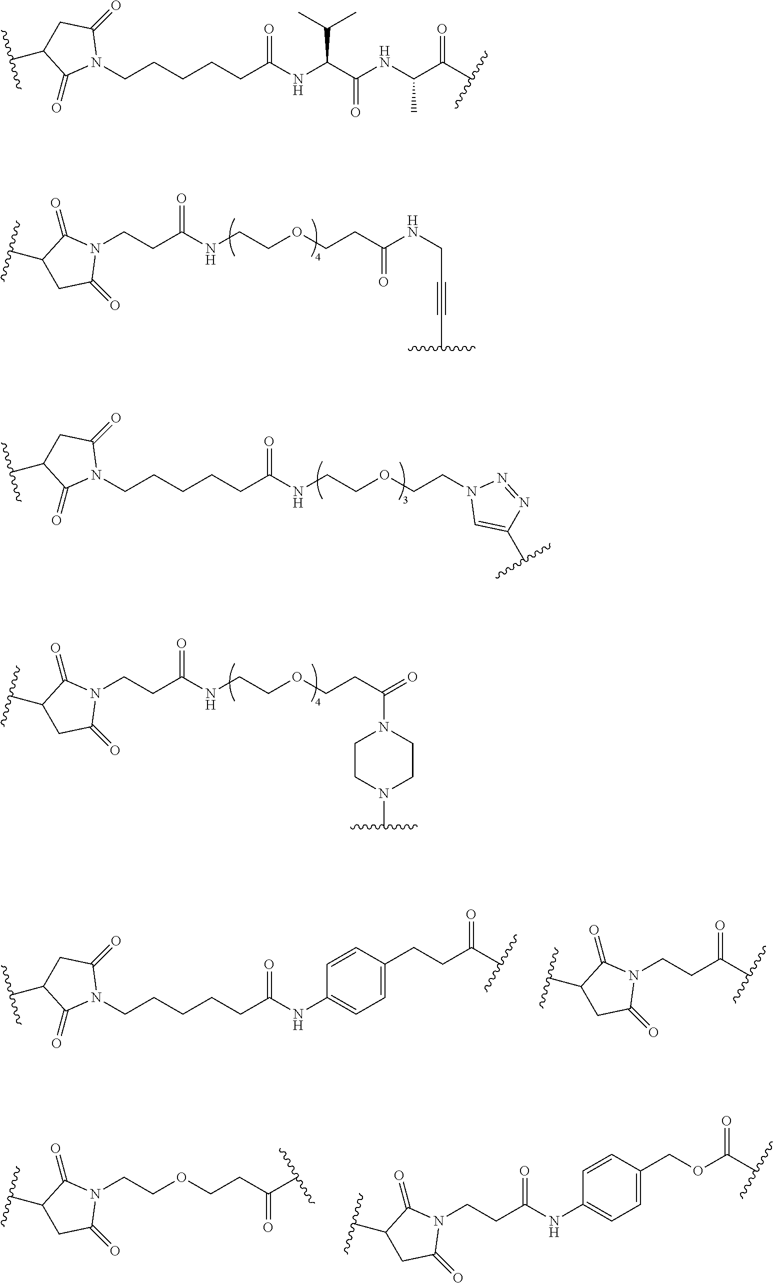

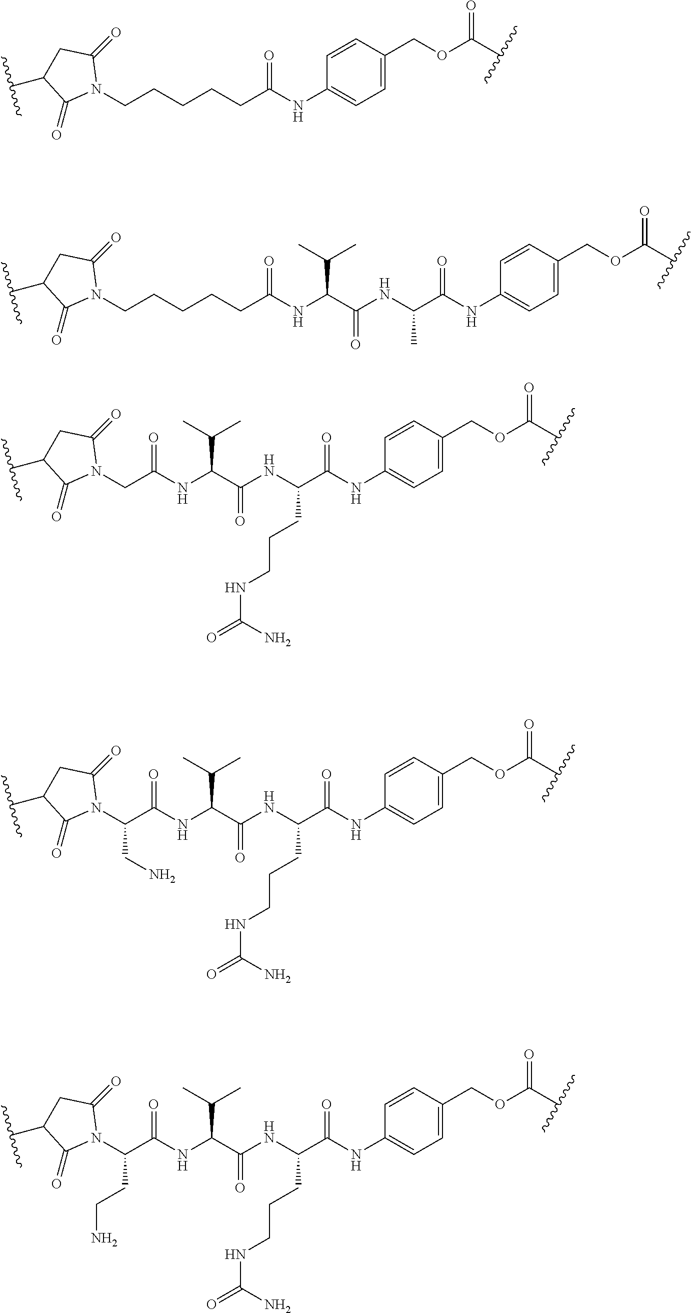 [SMILES: *C(=O)OCc1ccc(NC(=O)CCCCCN2C(=O)CC(*)C2=O)cc1 ; *C(=O)OCc1ccc(NC(=O)[C@H](C)NC(=O)[C@@H](NC(=O)CCCCCN2C(=O)CC(*)C2=O)C(C)C)cc1 ; *C(=O)OCc1ccc(NC(=O)[C@H](CCCNC(N)=O)NC(=O)[C@@H](NC(=O)CN2C(=O)CC(*)C2=O)C(C)C)cc1 ; *C(=O)OCc1ccc(NC(=O)[C@H](CCCNC(N)=O)NC(=O)[C@@H](NC(=O)[C@H](CN)N2C(=O)CC(*)C2=O)C(C)C)cc1 ; *C(=O)OCc1ccc(NC(=O)[C@H](CCCNC(N)=O)NC(=O)[C@@H](NC(=O)[C@H](CCN)N2C(=O)CC(*)C2=O)C(C)C)cc1]

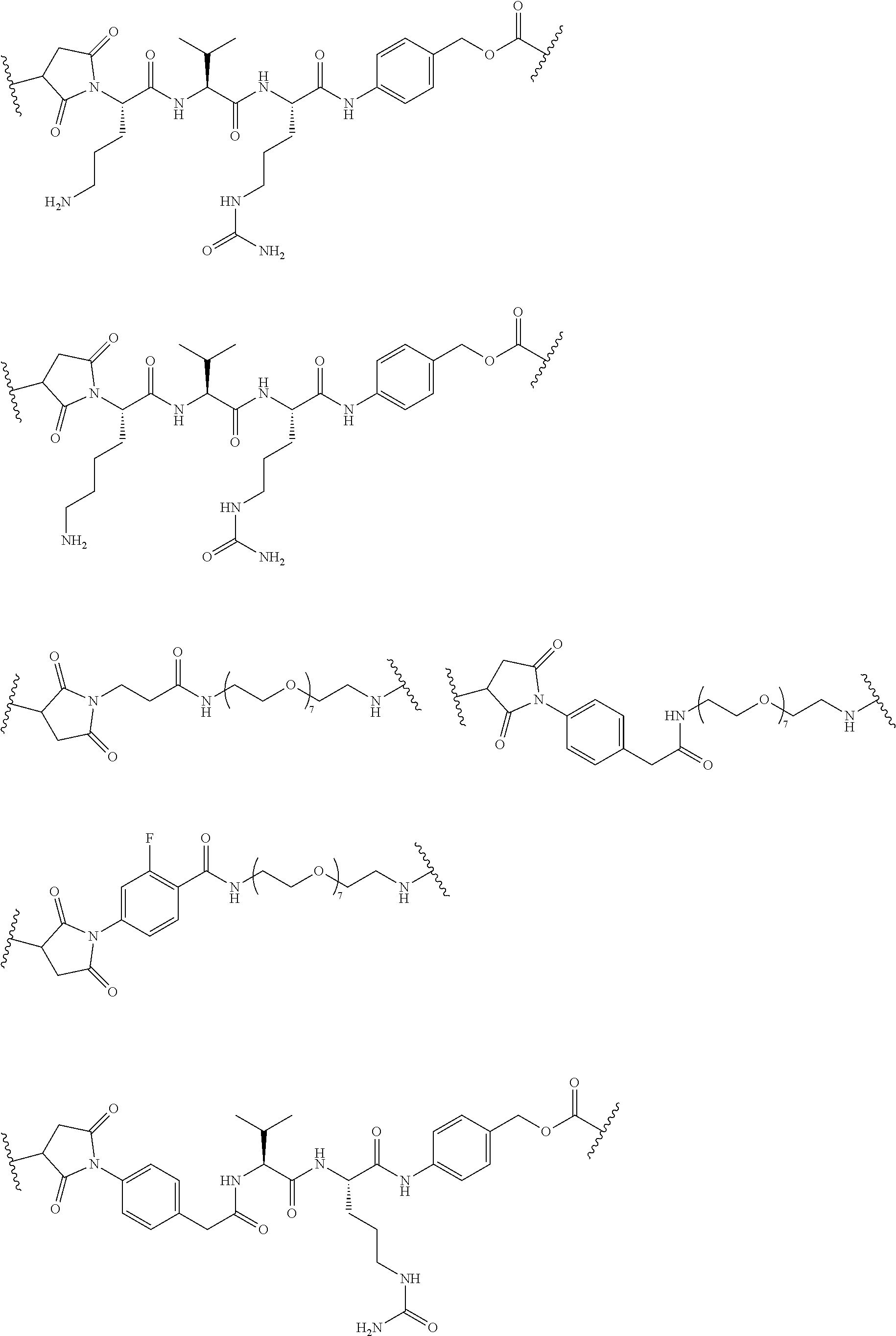

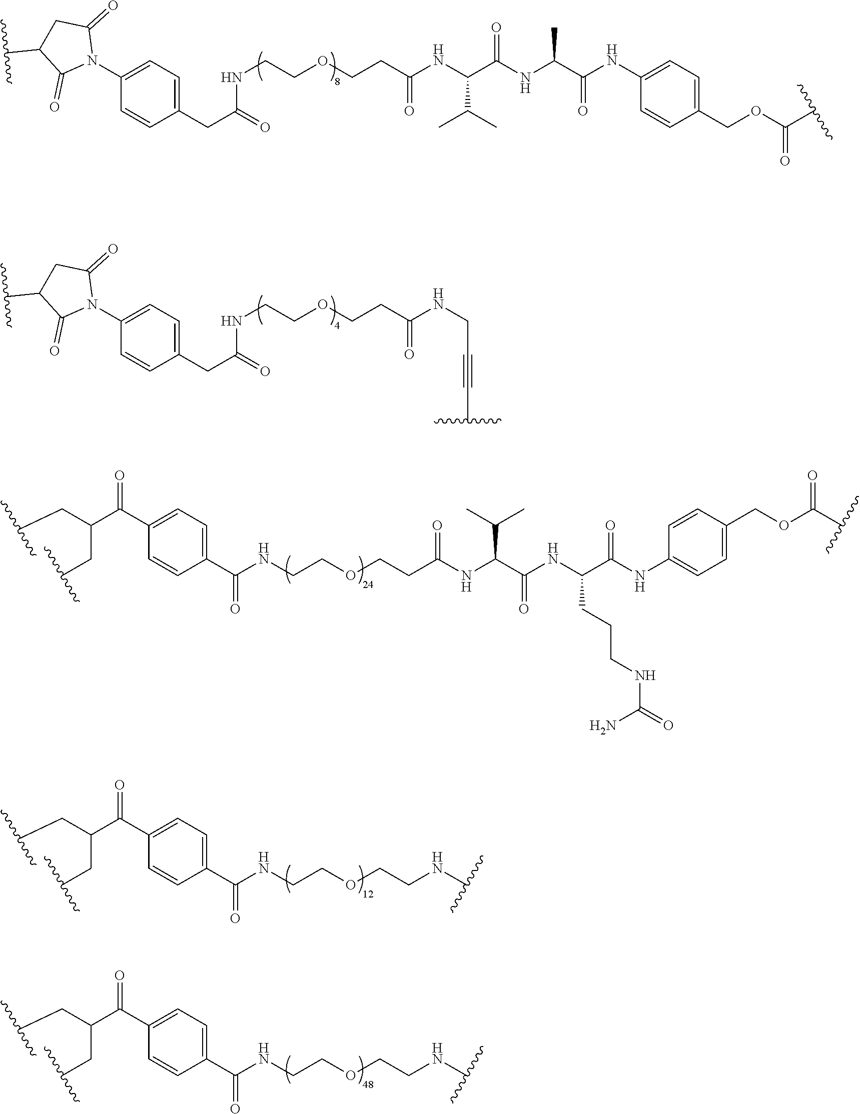

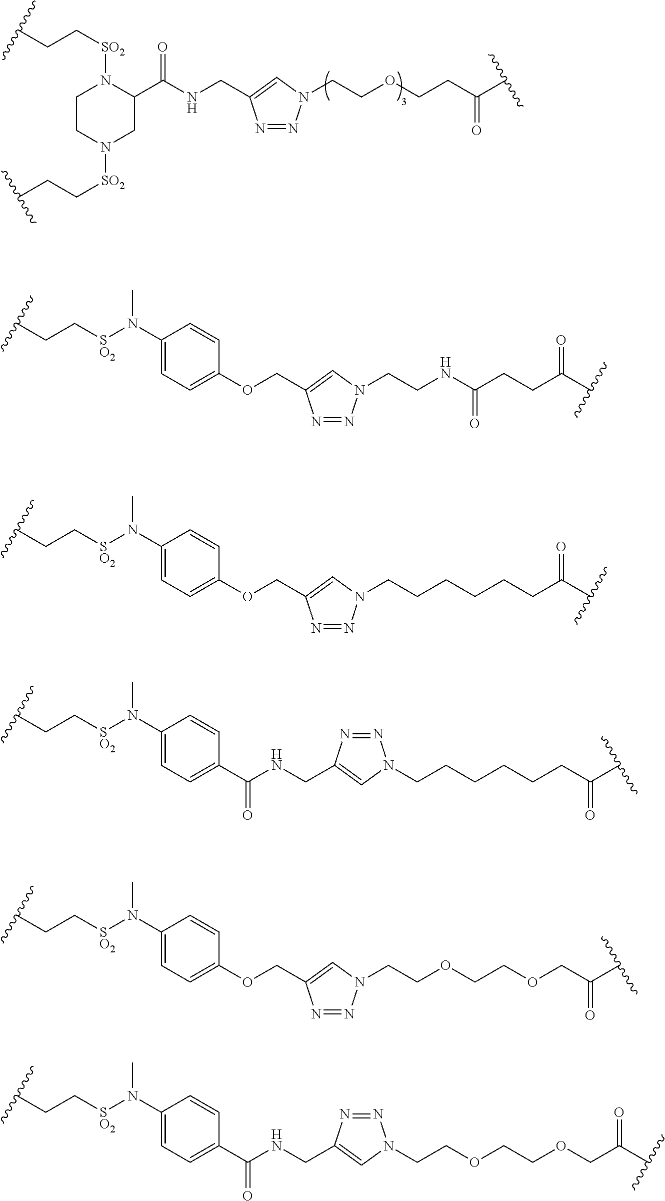

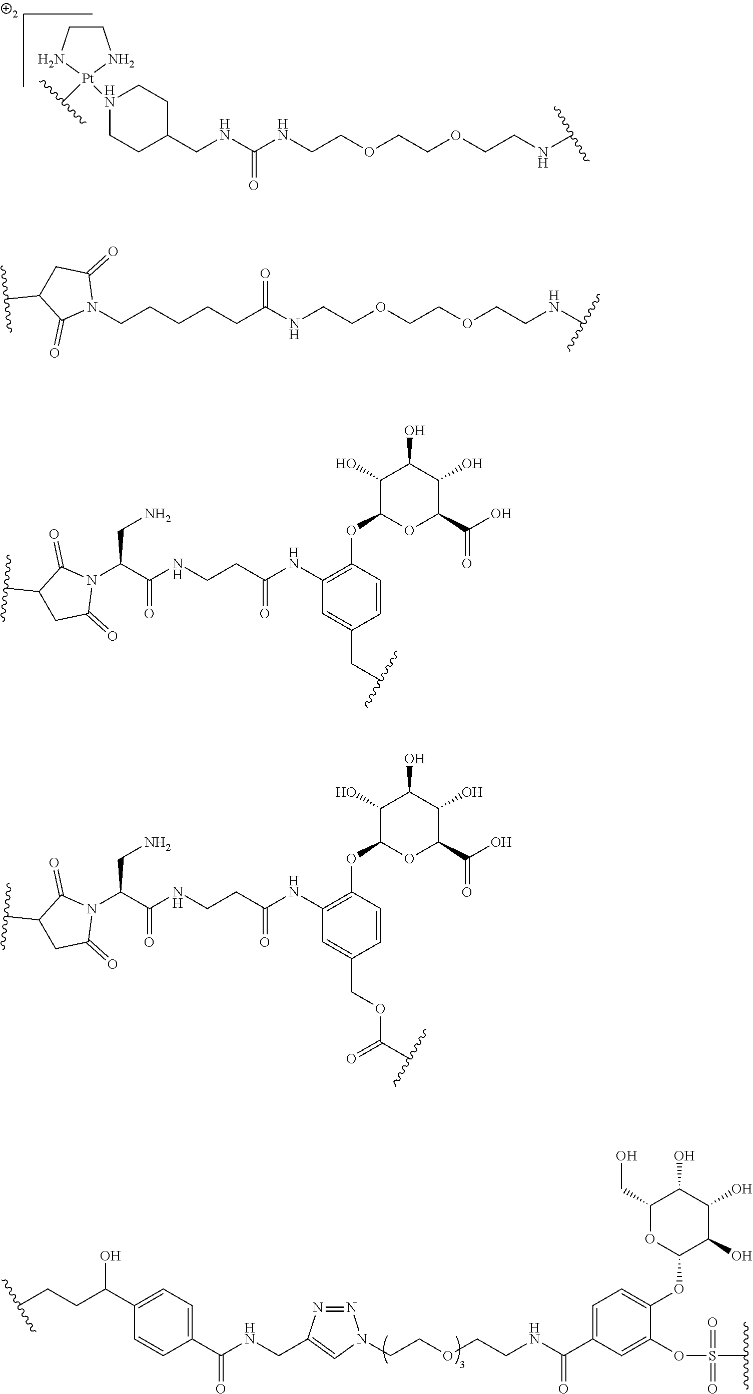

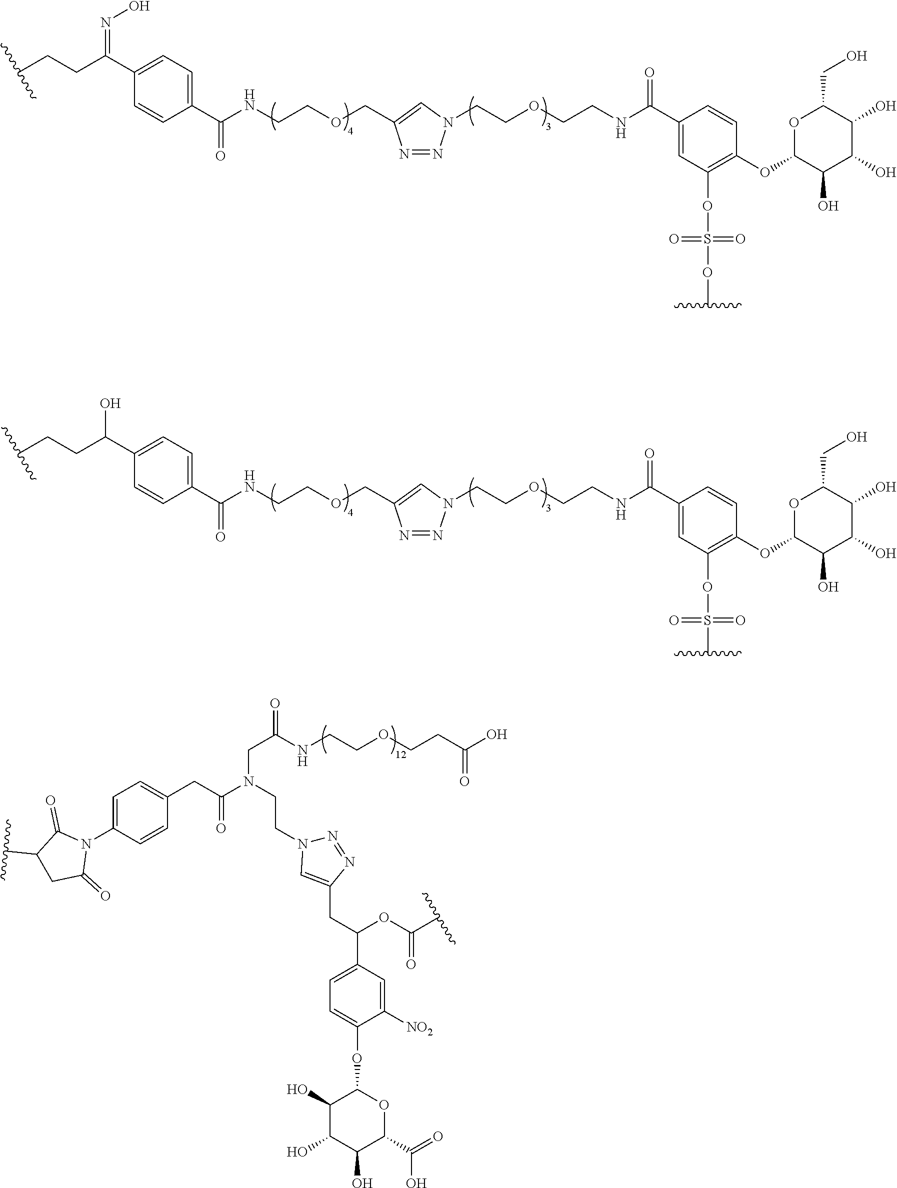

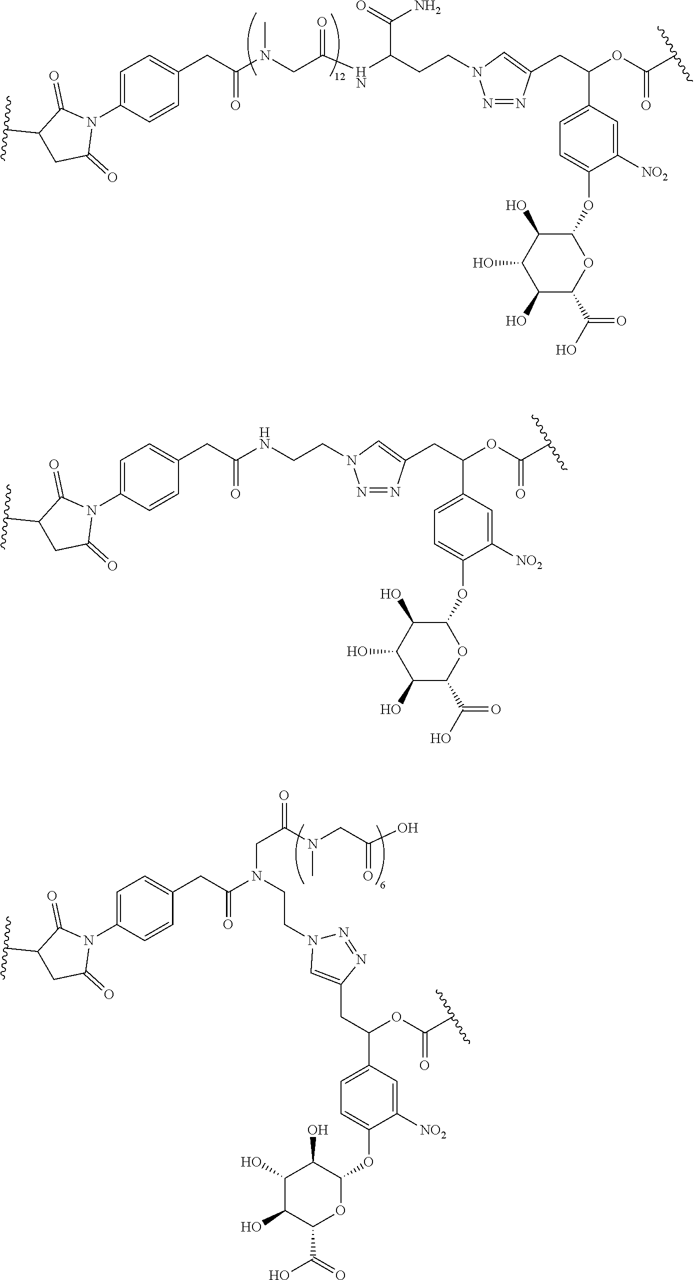

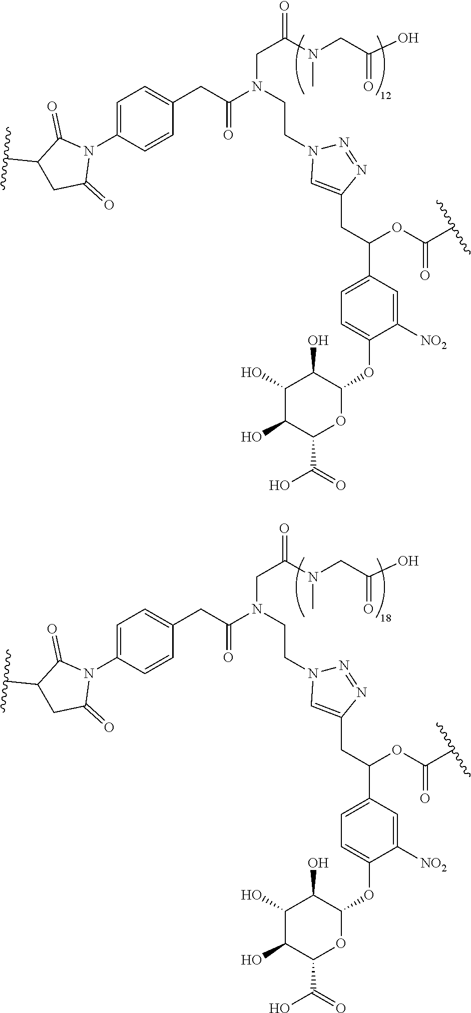

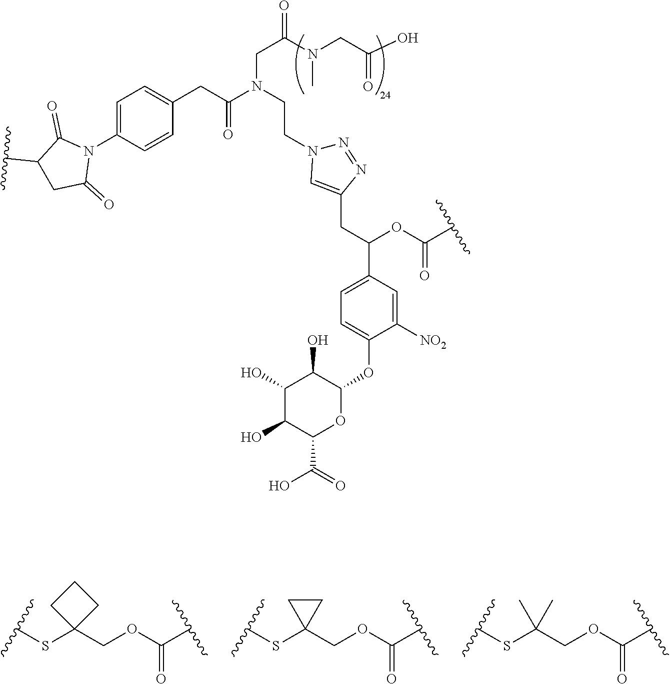

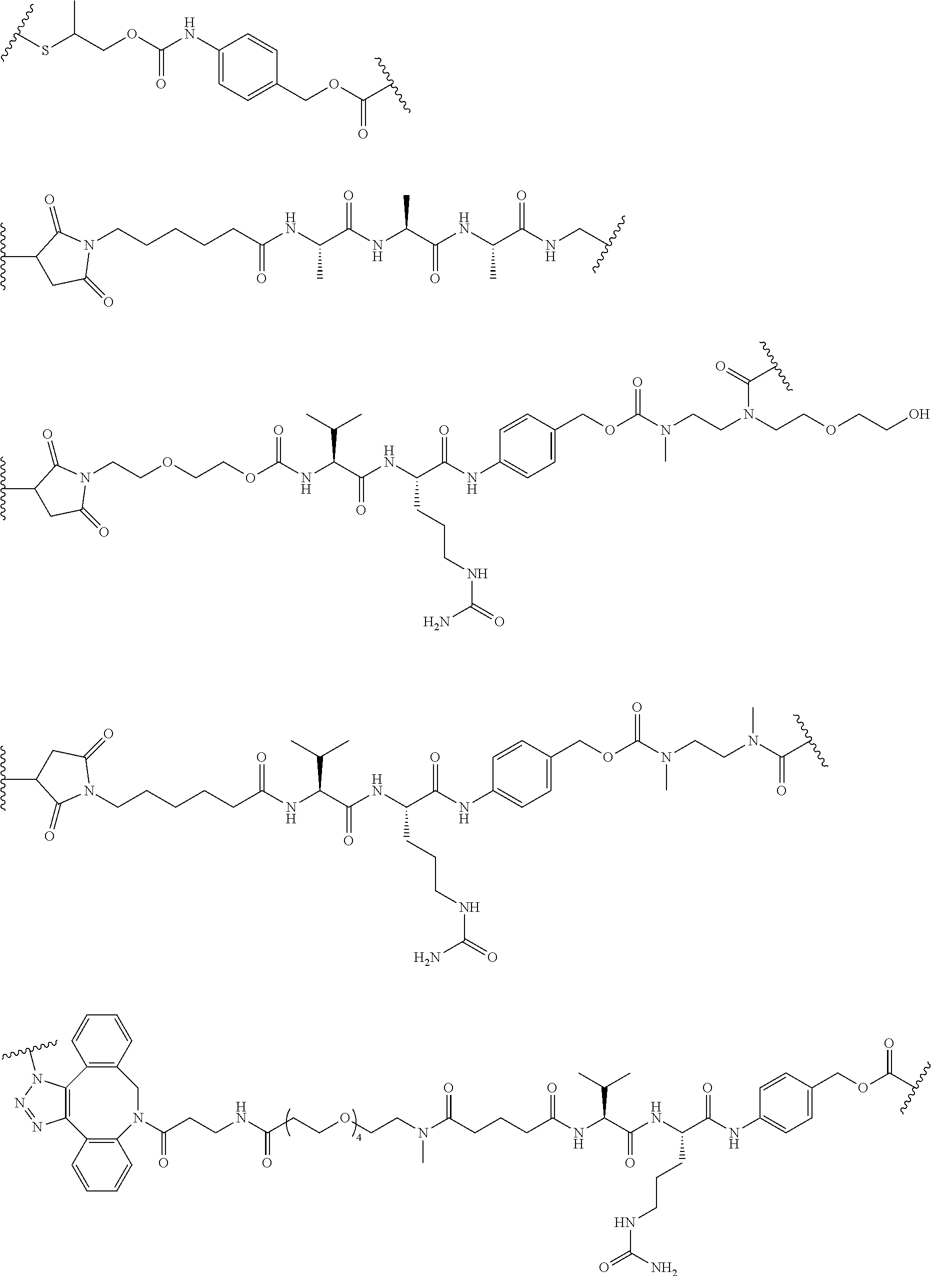

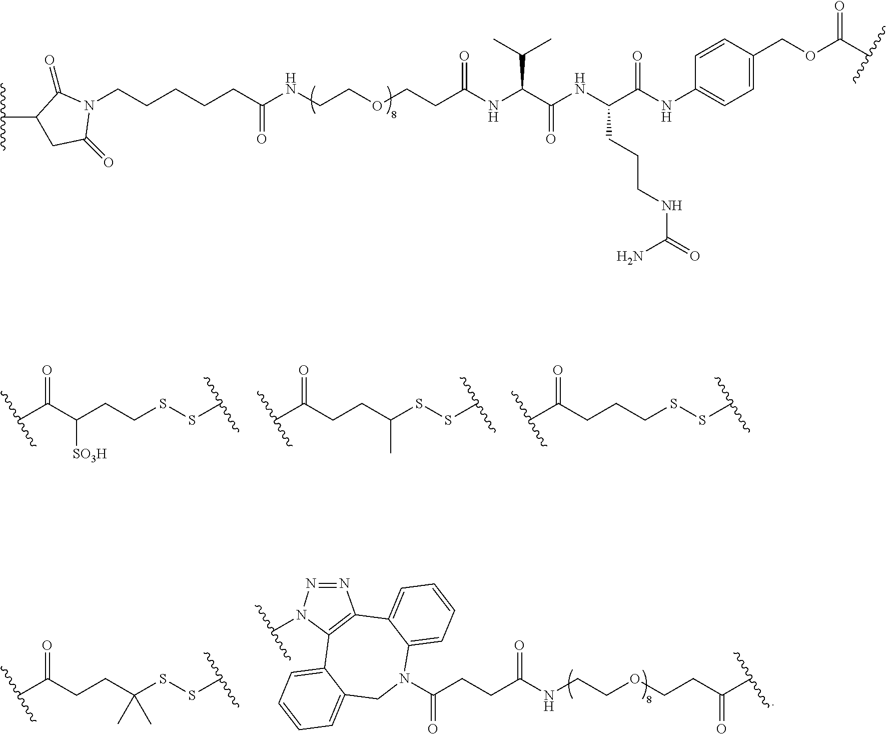

43. The conjugate of any one of claims 39 to 41, wherein the linker is a non-cleavable linker.

44. The conjugate of claim 43, wherein the non-cleavable linker is conjugated to a lysine of the antibody or antigen-binding fragment thereof.

45. The conjugate of claim 43, wherein the non-cleavable linker is conjugated to a cysteine of the antibody or antigen-binding fragment thereof.

46. The conjugate of claim 43, wherein the non-cleavable linker is conjugated to a histidine of the antibody or antigen-binding fragment thereof.

47. The conjugate of claim 43, wherein the non-cleavable linker is conjugated to an arginine of the antibody or antigen-binding fragment thereof.

48. The conjugate of claim 43, wherein the non-cleavable linker is conjugated to an aspartic acid of the antibody or antigen-binding fragment thereof.

49. The conjugate of claim 43, wherein the non-cleavable linker is conjugated to a glutamine of the antibody or antigen-binding fragment thereof.

50. The conjugate of claim 49, wherein the conjugate has a drug to antibody ratio (DAR) of at least 4:1.

51. The conjugate of claim 49 or 50, wherein the linker is a branched linker attached to at least two copies of the oligonucleotide moiety.

52. The conjugate of claim 43, wherein the non-cleavable linker is conjugated to a serine of the antibody or antigen-binding fragment thereof.

53. The conjugate of claim 43, wherein the non-cleavable linker is conjugated to a threonine of the antibody or antigen-binding fragment thereof.

54. The conjugate of claim 43, wherein the non-cleavable linker is conjugated to a tyrosine of the antibody or antigen-binding fragment thereof.

55. The conjugate of any one of claims 2 to 16, wherein each amino acid moiety of —X_{AA}— is independently selected from alanine (Ala), arginine (Arg), glycine (Gly), histidine (His), isoleucine (Ile), leucine (Leu), lysine (Lys), phenylalanine (Phe), tryptophan (Trp), tyrosine (Tyr), valine (Val), citrulline (Cit), and homocitrulline (HoCit).

56. The conjugate of any one of claims 2 to 16, wherein each amino acid moiety of —X_{AA}— is independently selected from alanine (Ala), glycine (Gly), lysine (Lys), phenylalanine (Phe), valine (Val), and citrulline (Cit).

57. The conjugate of any one of claims 2 to 15, wherein the amino acid sequence —X_{AA}— is selected from -Val-Cit-, -Cit-Val-, -Val-Ala-, -Ala-Val-, -Phe-Lys-, -Lys-Phe-, -Ala-Ala-, -Val-Val-, -Gly-Gly-, -Ala-Ala-Ala-, -Gly-Gly-Gly-, -Gly-Gly-Phe-Gly-, -Gly-Phe-Gly-Gly-, -Gly-Gly-Gly-Phe-, -Phe-Gly-Gly-Gly-, and -Gly-Gly-Gly-Gly-.

58. The conjugate of any one of claims 2 to 16, wherein the amino acid sequence —X_{AA}— is selected from -Val-Cit-, -Cit-Val-, -Val-Ala-, -Ala-Val-, -Phe-Lys-, -Lys-Phe-, -Ala-Ala-, -Val-Val-, -Gly-Gly-, -Ala-Ala-Ala-, and -Gly-Gly-Gly-.

59. The conjugate of any one of claims 1 to 58, wherein the payload moiety P is a drug moiety.

60. The conjugate of claim 59, wherein the drug moiety is selected from any of the following:

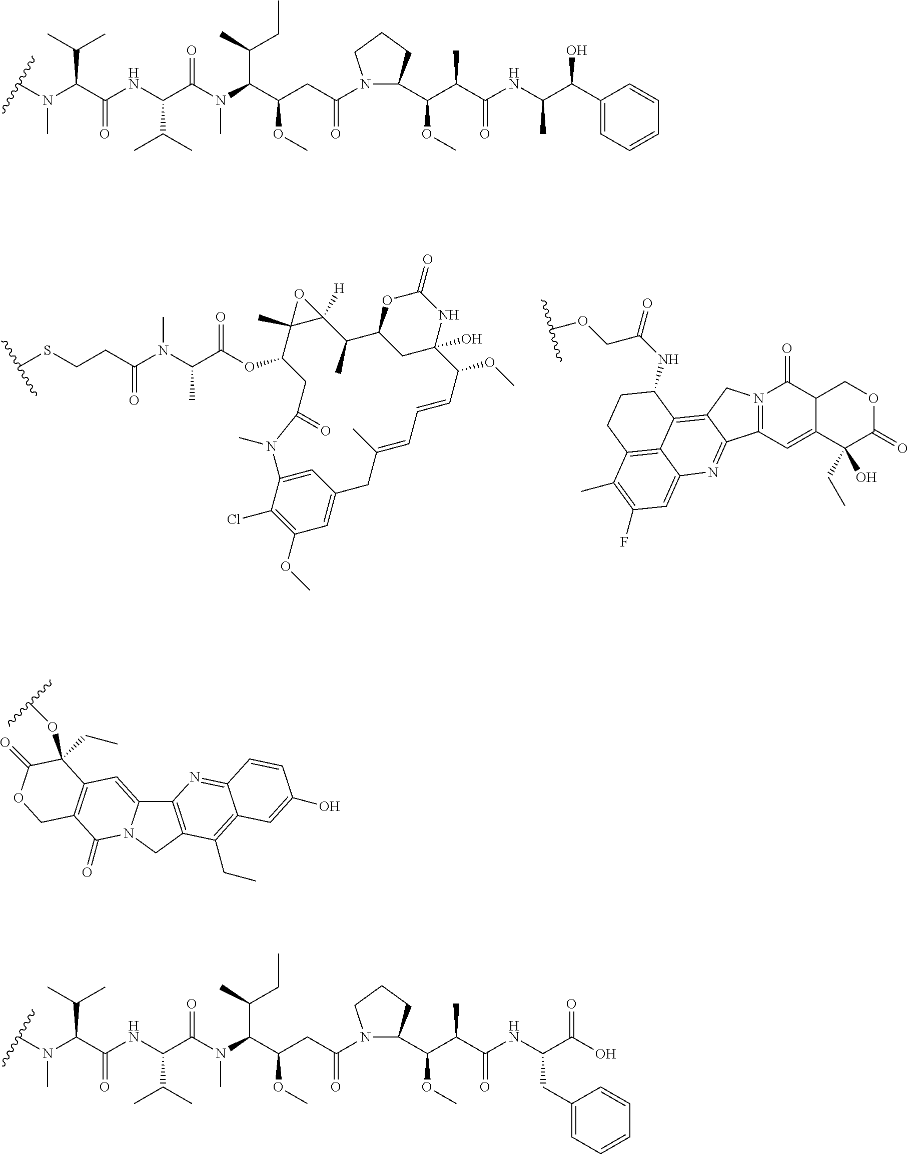

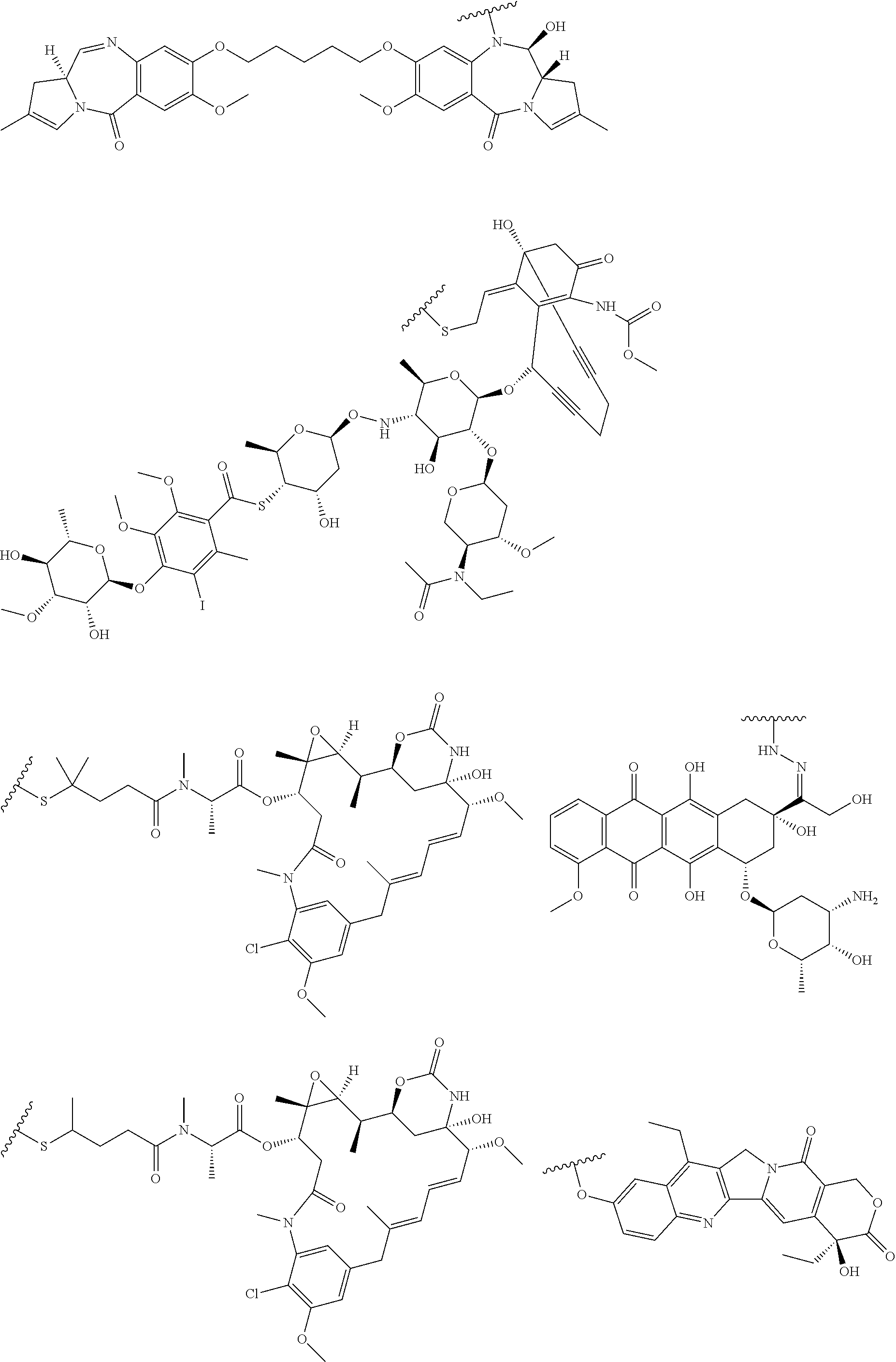

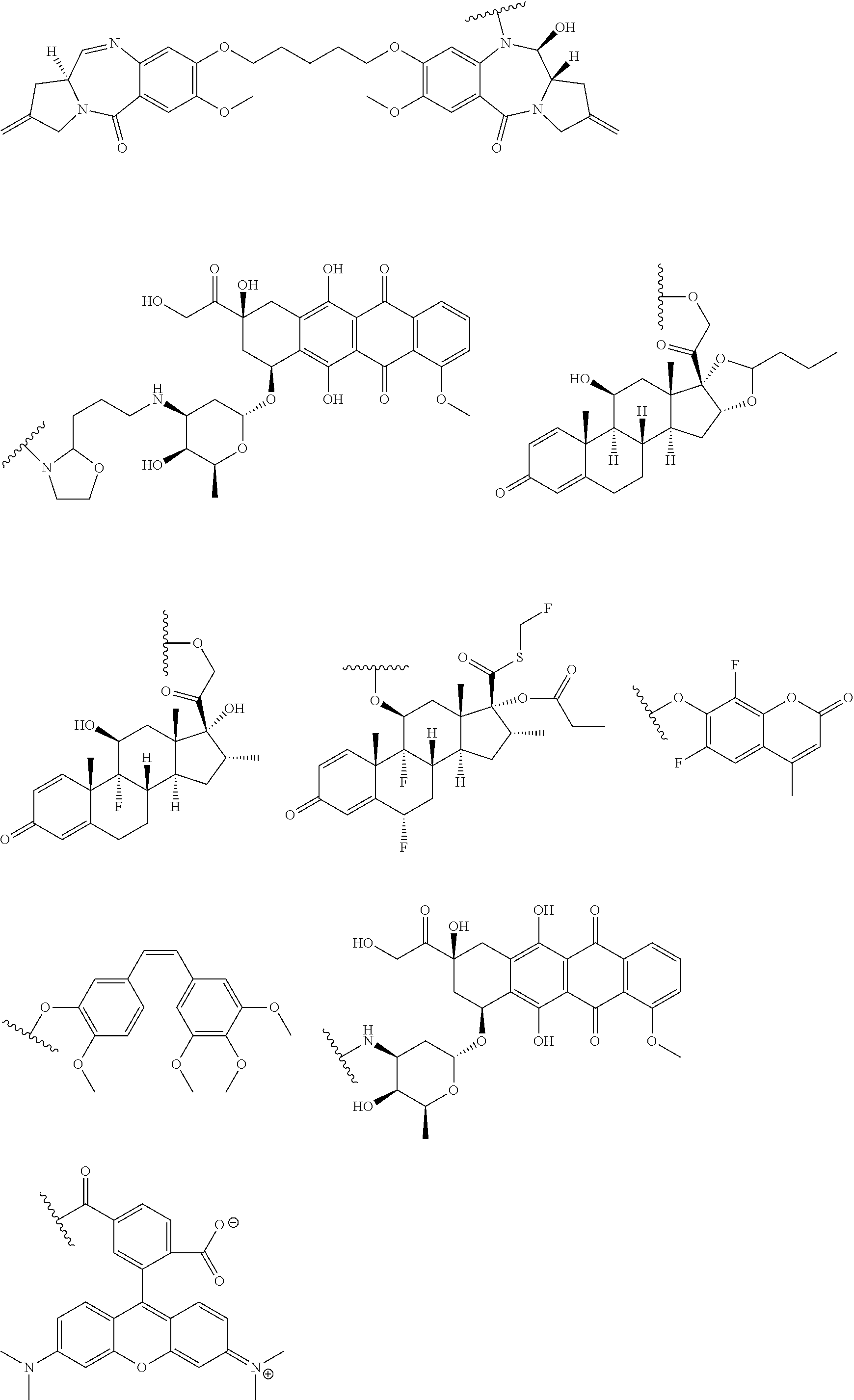

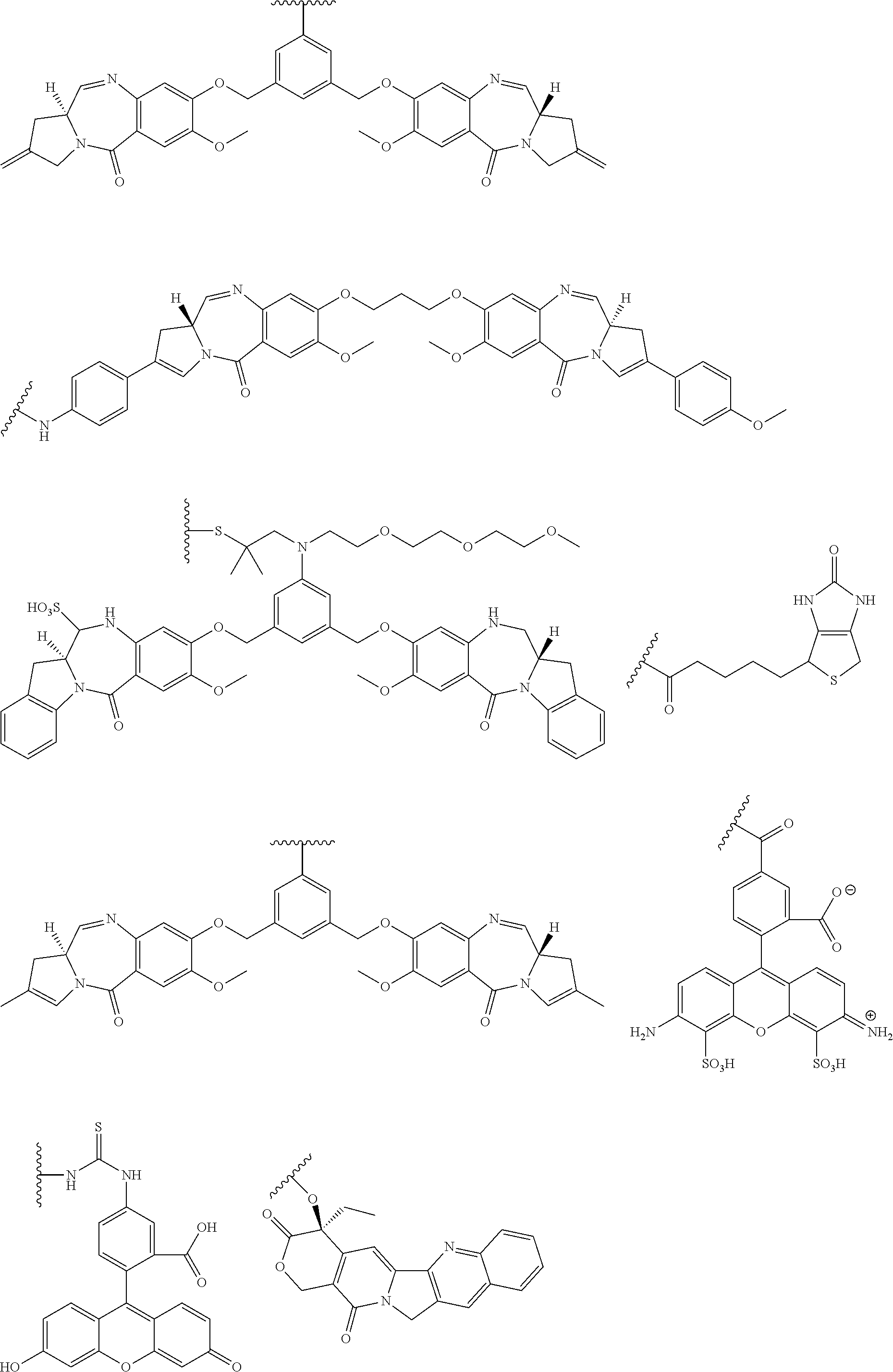

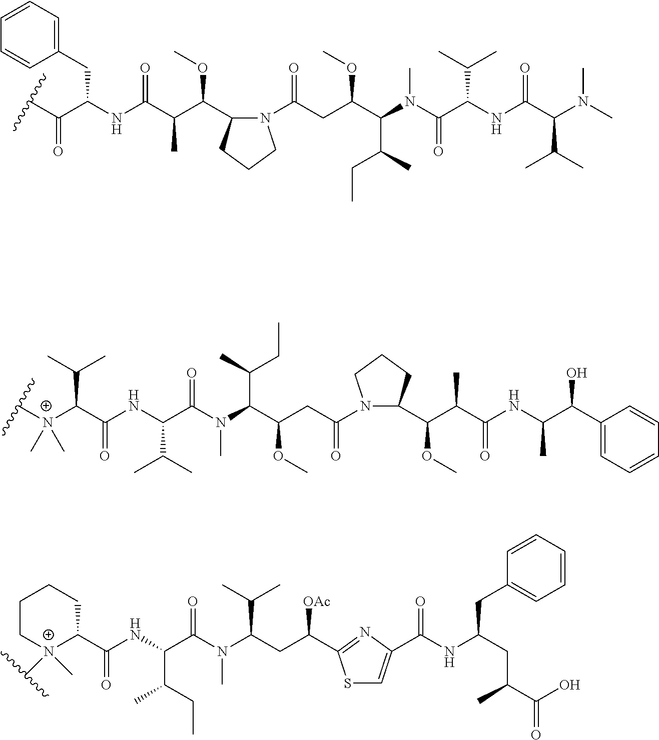

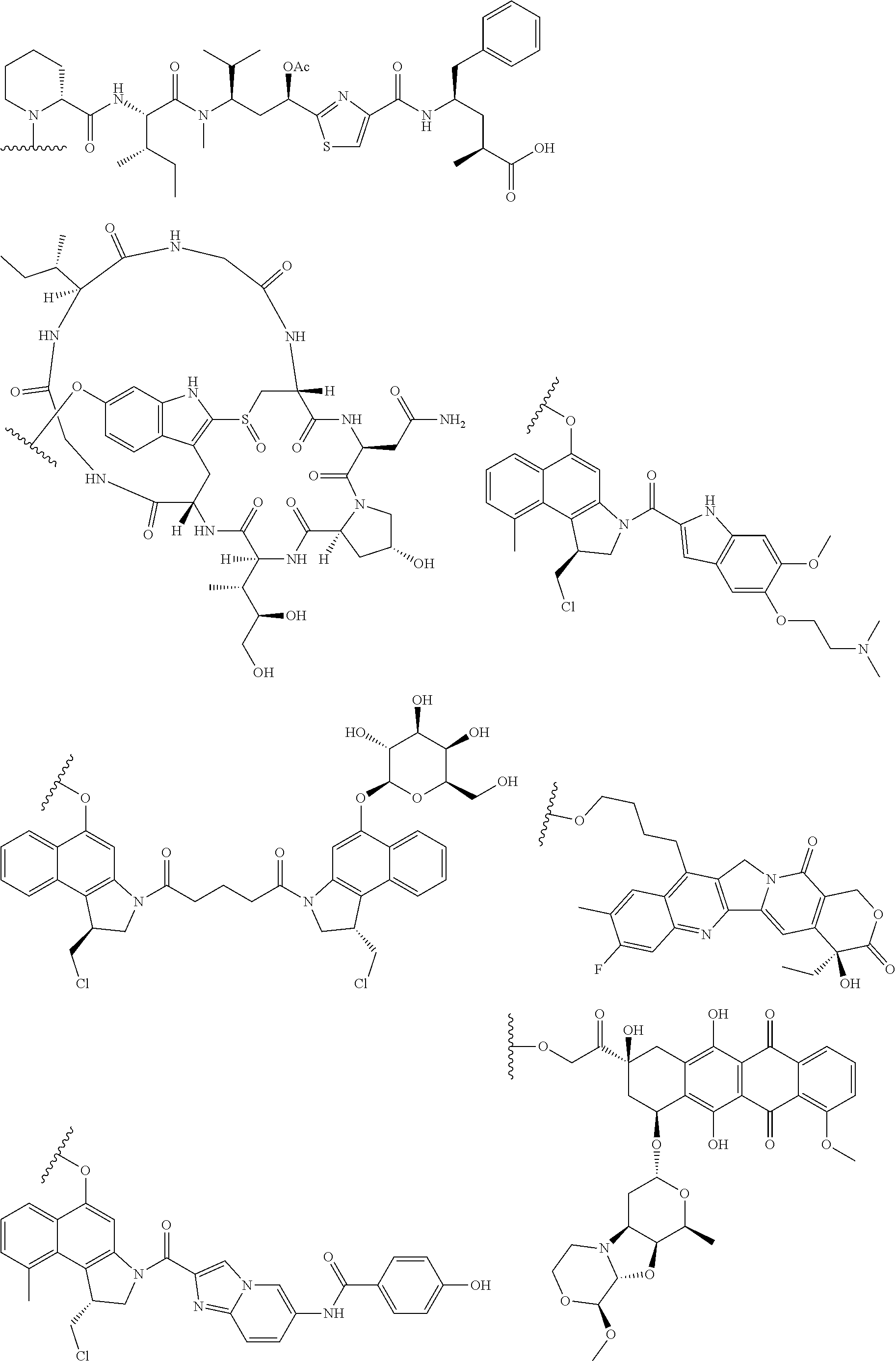

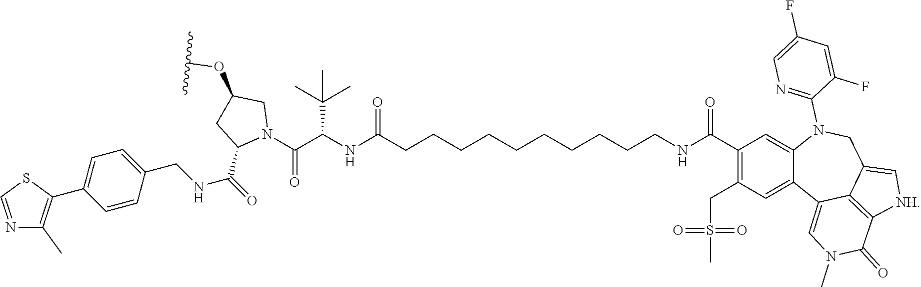 [SMILES: *O[C@@H]1C[C@@H](C(=O)NCc2ccc(-c3scnc3C)cc2)N(C(=O)[C@@H](NC(=O)CCCCCCCCCCNC(=O)c2cc3c(cc2CS(C)(=O)=O)-c2cn(C)c(=O)c4[nH]cc(c24)CN3c2ncc(F)cc2F)C(C)(C)C)C1]

61. The conjugate of any one of claims 1 to 60, wherein the VL CDR1 comprises the amino acid sequence of SEQ ID NO:9, CDR2 comprises the amino acid sequence of SEQ ID NO: 10, CDR3 comprises SEQ ID NO: 11; and the VH CDR1 comprises the amino acid sequence of SEQ ID NO: 15, CDR2 comprises the amino acid sequence of SEQ ID NO: 4, CDR3 comprises the amino acid sequence of SEQ ID NO:5.

62. The conjugate of any one of claims 1 to 60, wherein the VL CDR1 comprises the amino acid sequence of SEQ ID NO:29, CDR2 comprises the amino acid sequence of SEQ ID NO:10, CDR3 comprises SEQ ID NO:11; and the VH CDR1 comprises the amino acid sequence of SEQ ID NO:15, CDR2 comprises the amino acid sequence of SEQ ID NO:26, CDR3 comprises the amino acid sequence of SEQ ID NO:5.

63. The conjugate of any one of claims 1 to 62, wherein the antibody or antigen-binding fragment thereof comprises a light chain variable region (VL) comprising an amino acid sequence of SEQ ID NO:21 and a heavy chain variable region (VH) comprising an amino acid sequence of SEQ ID NO:14.

64. The conjugate of any one of claims 1 to 62, wherein the antibody or antigen-binding fragment thereof comprises a full length light chain (LC) comprising an amino acid sequence of SEQ ID NO:20 and a full length heavy chain (HC) comprising an amino acid sequence of SEQ ID NO:13.

65. The conjugate of any one of claims 1 to 62, wherein the antibody or antigen-binding fragment thereof comprises: a light chain variable domain (V_{L}) comprising an amino acid sequence that is at least about 95% identical to an amino acid sequence selected from the group consisting of 3E10-VL-H1 (SEQ ID NO:85), 3E10-VL-H2 (SEQ ID NO:86), 3E10-VL-H3 (SEQ ID NO:87), 3E10-VL-H4 (SEQ ID NO:88), 3E10-VL-H5 (SEQ ID NO:89), and 3E10-VL-H6 (SEQ ID NO:90); and a heavy chain variable domain (V_{H}) comprising an amino acid sequence that is at least about 95% identical to an amino acid sequence selected from the group consisting of 3E10-VH-H1 (SEQ ID NO:64), 3E10-VH-H2 (SEQ ID NO:65), 3E10-VH-H3 (SEQ ID NO:66), 3E10-VH-H4 (SEQ ID NO:67), 3E10-VH-H5 (SEQ ID NO:68), 3E10-VH-H6 (SEQ ID NO:69), and 3E10-VH-H7 (SEQ ID NO:70).

66. The conjugate of any one of claims 1 to 62, wherein the antibody or antigen-binding fragment thereof comprises: a light chain variable domain (V_{L}) comprising an amino acid sequence selected from the group consisting of 3E10-VL-H1 (SEQ ID NO:85), 3E10-VL-H2 (SEQ ID NO:86), 3E10-VL-H3 (SEQ ID NO:87), 3E10-VL-H4 (SEQ ID NO:88), 3E10-VL-H5 (SEQ ID NO:89), and 3E10-VL-H6 (SEQ ID NO:90); and a heavy chain variable domain (VH) comprising an amino acid sequence selected from the group consisting of 3E10-VH-H1 (SEQ ID NO:64), 3E10-VH-H2 (SEQ ID NO:65), 3E10-VH-H3 (SEQ ID NO:66), 3E10-VH-H4 (SEQ ID NO:67), 3E10-VH-H5 (SEQ ID NO:68), 3E10-VH-H6 (SEQ ID NO:69), and 3E10-VH-H7 (SEQ ID NO:70).

67. The conjugate of any one of claims 1 to 62, wherein the antibody or antigen-binding fragment thereof comprises a VL/VH pair selected from the group consisting of (a) VL1 (SEQ ID NO:85) and VH1 (SEQ ID NO:64), (b) VL1 (SEQ ID NO:85) and VH2 (SEQ ID NO:65), (c) VL1 (SEQ ID NO:85) and VH3 (SEQ ID NO:66), (d) VL1 (SEQ ID NO:85) and VH4 (SEQ ID NO:67), (e) VL2 (SEQ ID NO:86) and VH1 (SEQ ID NO:64), (f) VL2 (SEQ ID NO:86) and VH2 (SEQ ID NO:65), (g) VL2 (SEQ ID NO:86) and VH3 (SEQ ID NO:66), (h) VL2 (SEQ ID NO:86) and VH4 (SEQ ID NO:67), (i) VL3 (SEQ ID NO:87) and VH1 (SEQ ID NO:64), (j) VL3 (SEQ ID NO:87) and VH2 (SEQ ID NO:65), (k) VL3 (SEQ ID NO:87) and VH3 (SEQ ID NO:66), (l) VL3 (SEQ ID NO:87) and VH4 (SEQ ID NO:67), (m) VL4 (SEQ ID NO:88) and VH1 (SEQ ID NO:64), (n) VL4 (SEQ ID NO:88) and VH2 (SEQ ID NO:65), (o) VL4 (SEQ ID NO:88) and VH3 (SEQ ID NO:66), (p) VL4 (SEQ ID NO:88) and VH4 (SEQ ID NO:67), (q) VL5 (SEQ ID NO:89) and VH5 (SEQ ID NO:68), (r) VL5 (SEQ ID NO:89) and VH6 (SEQ ID NO:69), (s) VL6 (SEQ ID NO:90) and VH5 (SEQ ID NO:68), and (t) VL6 (SEQ ID NO:90) and VH6 (SEQ ID NO:69).

68. The conjugate of any one of claims 1 to 60, wherein the antibody or antigen-binding fragment thereof comprises: a light chain variable domain (VL) comprising 3E10-VL-H6 (SEQ ID NO:90) and a heavy chain variable domain (VH) comprising 3E10-VH-H6 (SEQ ID NO:69).

69. The conjugate of any one of claims 1 to 60, wherein the antibody or antigen-binding fragment thereof comprises: a light chain variable domain (VL) comprising the amino acid sequence (DIQMTQSPSSLSASLGDRATITCRASKTVSTSSYSYMHWYQQKPGQPPKLLIKYASYLE SGVPSRFSGSGSGTDFTLTISSLQPEDAATYYCQHSREFPWTFGGGTKVEIK) (SEQ ID NO:126) and a heavy chain variable domain (VH) comprising the amino acid sequence (EVQLVESGGGLVQPGGSLRLSCAASGFTFSNYGMHWVRQAPGKGLEWVSYISSGSSTI YYADSVKGRFTISRDNAKNSLYLQMNSLRAEDTAVYYCARRGLLLDYWGQGTTVTVS S) (SEQ ID NO:127).

70. A method for treating a subject in need thereof, the method comprising administering a therapeutically effective amount of a conjugate according to any one of claims 1 to 69 to the subject.

71. A method for treating cancer in a subject in need thereof, the method comprising administering to the subject a therapeutically effective amount of a conjugate according to any one of claims 1 to 69 to the subject.

72. The method of claim 71, wherein the cancer is a carcinoma, a sarcoma, a blastoma, a papilloma, or an adenoma.

73. The method of claim 71, wherein the cancer is metastatic cancer.

74. The method of any one of claims 71-73, wherein the cancer is selected from the group consisting of bladder cancer, blood cancer, brain cancer, breast cancer, bone cancer, cervical cancer, colorectal cancer, endocrine cancer, esophageal cancer, gastric cancer, head and neck cancer, hepatobiliary cancer, leukemia, lung cancer, lymphoma, melanoma, myeloma, ovarian cancer, pancreatic cancer, prostate cancer, renal cancer, thyroid cancer, and uterine cancer.

75. The method of claim 74, wherein the cancer is a skin cancer selected from the group consisting of basal cell carcinoma, squamous cell carcinoma, and melanoma.

76. The method of claim 74, wherein the cancer is melanoma.

77. The method of claim 71, wherein the cancer is ovarian cancer.

78. The method of claim 71, wherein the cancer is colorectal cancer.

79. The method of claim 71, wherein the cancer is breast cancer.

80. The method of claim 71, wherein the cancer is BRCA2- breast cancer.

81. The method of claim 71, wherein the cancer is triple negative breast cancer.

82. The method of claim 71, wherein the cancer is lung cancer.

83. The method of claim 71, wherein the cancer is non-small cell lung cancer (NSCLC).

84. The method of any one of claims 71-73, wherein the cancer is a cancer of the central nervous system.

85. The method of claim 71, wherein the cancer is a neuroepithelial brain or spinal tumor selected from the group consisting of a medulloblastoma, an astrocytic tumor, an oligodendroglial tumor, an oligoastrocytic tumor, an ependymal tumor, a choroid plexus tumor, a neuronal or mixed neuronal-glial tumor, a tumor of the pineal region, an embryonal tumor, or an otherwise uncategorized neuroepithelial tumor.

86. The method of any one of claims 71-84, wherein the administering is by parenteral administration.

87. The method of claim 85, wherein the parenteral administration is intramuscular administration, intravenous administration, or subcutaneous administration.
